(19) United States
(12) Patent Application Publication
(10) Pub. No.: US 2021/0087568 A1
(43) Pub. Date: Mar. 25, 2021

(54) Modified Guide RNAs for Gene Editing

(71) Applicant: Intellia Therapeutics, Inc., Cambridge, MA (US)

(72) Inventors: Amy Madison Rhoden Smith, Durham, NC (US); Seth C. Alexander, Medford, MA (US); Duncan Brown, Berkeley, CA (US); Robert Brown, Cambridge, MA (US); Adhiraj Lanba, Medford, MA (US); Rebecca Lescarbeau, Medford, MA (US); Reynald Michael Lescarbeau, Medford, MA (US); Rubina Parmar, Acton, MA (US); Matthew Roy, Arlington, MA (US); Paige Salerno, Arlington, MA (US); Jessica Lynn Seitzer, Windham, NH (US)

(73) Assignee: Intellia Therapeutics, Inc., Cambridge, MA (US)

(21) Appl. No.: 17/111,769

(22) Filed: Dec. 4, 2020

Related U.S. Application Data
(63) Continuation of application No. PCT/US2019/036160, filed on Jun. 7, 2019.
(60) Provisional application No. 62/682,838, filed on Jun. 8, 2018.
(60) Provisional application No. 62/682,820, filed on Jun. 8, 2018.

(51) Int. Cl. C12N 15/113; C12N 9/22

(52) U.S. Cl. CPC C12N 15/113; C12N 9/22; C12N 2310/531; C12N 2310/20; C12N 2310/315; C12N 2310/3231; C12N 2310/321

(57) ABSTRACT

This disclosure relates to modified guide RNAs having improved in vitro and in vivo activity in gene editing methods.

This application is a Continuation of International Application No. PCT/US2019/036160, filed on Jun. 7, 2019, which claims the benefit of U.S. Provisional Patent Application No. 62/682,838, filed Jun. 8, 2018, and U.S. Provisional Patent Application No. 62/682,820, filed Jun. 8, 2018, each of which is incorporated herein by reference for all purposes.

The instant application contains a Sequence Listing which has been submitted electronically in ASCII format and is hereby incorporated by reference in its entirety. Said ASCII copy, created on Dec. 4, 2020, is named 2020-12-04_01155-0022-00US_ST25.txt and is 969,230 bytes in size.

This disclosure relates to the field of gene editing using CRISPR/Cas systems, a part of the prokaryotic immune system that recognizes and cuts exogenous genetic elements. The CRISPR/Cas system relies on a single nuclease, termed CRISPR-associated protein 9 (Cas9), which induces site-specific breaks in DNA. Cas9 is guided to specific DNA sequences by small RNA molecules termed guide RNA (gRNA). A complete guide RNA comprises tracrRNA (trRNA) and crisprRNA (crRNA). A crRNA comprising a guide region may also be referred to as a gRNA, with the understanding that to form a complete gRNA it should be or become associated covalently or noncovalently with a trRNA. The trRNA and crRNA may be contained within a single guide RNA (sgRNA) or in two separate RNA molecules of a dual guide RNA (dgRNA). Cas9 in combination with trRNA and crRNA or an sgRNA is termed the Cas9 ribonucleoprotein complex (RNP).

Oligonucleotides, and in particular RNA, are sometimes degraded in cells and in serum by non-enzymatic, endonuclease or exonuclease cleavage. Improved methods and compositions for preventing such degradation, improving stability of gRNAs and enhancing gene editing efficiency is desired, especially for therapeutic applications.

### SUMMARY

In some embodiments, genome editing tools are provided comprising modified guide RNA (gRNA). The modifications of gRNAs described herein may improve the stability of the gRNA and the gRNA/Cas9 complex and improve the activity of Cas9 (e.g., SaCas9, SpyCas9, and equivalents) to cleave target DNA.

In some embodiments, modified crisprRNA (crRNA) and/or modified tracrRNA (trRNA) are provided. In some embodiments, the modified crRNA and/or modified trRNA comprise a dual guide RNA (dgRNA). In some embodiments, the modified crRNA and/or modified trRNA comprise a single guide RNA (sgRNA). The modifications of crRNA and/or trRNA described herein may improve the stability of the gRNA and the gRNA/Cas9 complex and improve the activity of Cas9 (e.g., SauCas9, SpyCas9, and equivalents) to cleave target DNA. In some embodiments, the crRNA portion of a dgRNA or an sgRNA is modified in the targeting domain.

In some embodiments, genome editing tools are provided comprising short-single guide RNA (short-sgRNA). In some embodiments, the short-sgRNA is modified. The short-sgRNAs described herein may improve the stability of the short-sgRNA and the short-sgRNA/Cas9 complex and improve the activity of Cas9 (e.g., SauCas9, SpyCas9, and equivalents) to cleave target DNA.

In some embodiments, a gRNA (e.g., sgRNA, short-sgRNA, dgRNA, or crRNA) comprises a modification at one or more YA sites, e.g., as set forth in the embodiments below, Table 1, and in the Examples and associated Figures. For the avoidance of doubt, sgRNAs include but are not limited to short-sgRNAs. As discussed in the Examples section, it has been found that gRNAs can be susceptible to an RNase A-like degradation pattern, e.g., including cleavage at unmodified YA sites. It has further been found that YA site modifications can reduce or eliminate such cleavage and that many YA site modifications appear to be tolerated without adversely affecting the ability of the gRNA to direct cleavage by a nuclease such as Cas9. It has also been found that certain gRNA positions, including but not limited to YA sites, can be modified despite statements by others (see Yin et al., Nature Biotechnol. 35:1179-1187 (2017)) that they are contacted by Cas9 and should not be modified out of concern for loss of activity. Such modifications may further reduce undesirable gRNA degradation while not compromising activity.

The following embodiments are encompassed.
Embodiment 01 is a guide RNA (gRNA) which is a short-single guide RNA (short-sgRNA) comprising a conserved portion of an sgRNA comprising a hairpin region, wherein the hairpin region lacks at least 5-10 nucleotides and wherein the short-sgRNA comprises a 5′ end modification or a 3′ end modification.
Embodiment 02 is the gRNA of embodiment 1, wherein the short-sgRNA comprises a 5′ end modification.
Embodiment 03 is the gRNA of any one of the preceding embodiments, wherein the short-sgRNA comprises a 3′ end modification.
Embodiment 04 is the gRNA of any one of the preceding embodiments, wherein the short-sgRNA comprises a 5′ end modification and a 3′ end modification.
Embodiment 05 is the gRNA of any one of the preceding embodiments, wherein the short-sgRNA comprises a 3′ tail.
Embodiment 06 is the gRNA of embodiment 5, wherein the 3′ tail comprises 1, 2, 3, 4, 5, 6, 7, 8, 9, or 10 nucleotides.
Embodiment 07 is the gRNA of embodiment 5, wherein the 3′ tail comprises about 1-2, 1-3, 1-4, 1-5, 1-7, 1-10, at least 1-5, at least 1-3, at least 1-4, at least 1-5, at least 1-5, at least 1-7, or at least 1-10 nucleotides.
Embodiment 08 is the gRNA of any one of the preceding embodiments, wherein the short-sgRNA does not comprise a 3′ tail.
Embodiment 09 is the gRNA of any one of the preceding embodiments, comprising a modification in the hairpin region.
Embodiment 10 is the gRNA of any one of the preceding embodiments, comprising a 3′ end modification, and a modification in the hairpin region.
Embodiment 11 is the gRNA of any one of the preceding embodiments, comprising a 3′ end modification, a modification in the hairpin region, and a 5′ end modification.
Embodiment 12 is the gRNA of any one of the preceding embodiments, comprising a 5′ end modification, and a modification in the hairpin region.
Embodiment 13 is the gRNA of any one of the preceding embodiments, wherein the at least 5-10 lacking nucleotides are consecutive.
Embodiment 14 is the gRNA of any one of the preceding embodiments, wherein the at least 5-10 lacking nucleotides:
   i. are within hairpin 1;
   ii. are within hairpin 1 and the “N” between hairpin 1 and hairpin 2;
   iii. are within hairpin 1 and the two nucleotides immediately 3′ of hairpin 1;
   iv. include at least a portion of hairpin 1;
   v. are within hairpin 2;
   vi. include at least a portion of hairpin 2;
   vii. are within hairpin 1 and hairpin 2;
   viii. include at least a portion of hairpin 1 and include the “N” between hairpin 1 and hairpin 2;
   ix. include at least a portion of hairpin 2 and include the “N” between hairpin 1 and hairpin 2;
   x. include at least a portion of hairpin 1, include the “N” between hairpin 1 and hairpin 2, and include at least a portion of hairpin 2;
   xi. are within hairpin 1 or hairpin 2, optionally including the “N” between hairpin 1 and hairpin 2;
   xii. are consecutive;
   xiii. are consecutive and include the “N” between hairpin 1 and hairpin 2;
   xiv. are consecutive and span at least a portion of hairpin 1 and a portion of hairpin 2;
   xv. are consecutive and span at least a portion of hairpin 1 and the “N” between hairpin 1 and hairpin 2; or
   xvi. are consecutive and span at least a portion of hairpin 1 and two nucleotides immediately 3′ of hairpin 1.
Embodiment 15 is the gRNA of any one of the preceding embodiments, further comprising a guide region.
Embodiment 16 is the gRNA of any one of the preceding embodiments, wherein the 3′ and/or 5′ end modification comprises a protective end modification, such as a modified nucleotide selected from 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.
Embodiment 17 is the gRNA of any one of the preceding embodiments, wherein the modification in the hairpin region comprises a modified nucleotide selected from 2′-O-methyl (2′-OMe) modified nucleotide, a 2′-fluoro (2′-F) modified nucleotide, a phosphorothioate (PS) linkage between nucleotides, or combinations thereof.
Embodiment 18 is the gRNA of any one of the preceding embodiments, wherein the 3′ and/or 5′ end modification comprises or further comprises a 2′-O-methyl (2′-OMe) modified nucleotide.
Embodiment 19 is the gRNA of any one of the preceding embodiments, wherein the 3′ and/or 5′ end modification comprises or further comprises a 2′-fluoro (2′-F) modified nucleotide.
Embodiment 20 is the gRNA of any one of the preceding embodiments, wherein the 3′ and/or 5′ end modification comprises or further comprises a phosphorothioate (PS) linkage between nucleotides.
Embodiment 21 is the gRNA of any one of the preceding embodiments, wherein the 3′ and/or 5′ end modification comprises or further comprises an inverted abasic modified nucleotide.
Embodiment 22 is the gRNA of any one of the preceding embodiments, wherein the modification in the hairpin region comprises or further comprises a 2′-O-methyl (2′-OMe) modified nucleotide.
Embodiment 23 is the gRNA of any one of the preceding embodiments, wherein the modification in the hairpin region comprises or further comprises a 2′-fluoro (2′-F) modified nucleotide.
Embodiment 24 is the gRNA of any one of the preceding embodiments, wherein the 3′ end modification comprises any of:
   i. a modification of any one or more of the last 7, 6, 5, 4, 3, 2, or 1 nucleotides;
   ii. one modified nucleotide;
   iii. two modified nucleotides;
   iv. three modified nucleotides;
   v. four modified nucleotides;
   vi. five modified nucleotides;
   vii. six modified nucleotides; and
   viii. seven modified nucleotides.
Embodiment 25 is the gRNA of any one of the preceding embodiments, wherein the at least 5-10 nucleotides comprise nucleotides 54-61 of SEQ ID NO:400, nucleotides 53-60 of SEQ ID NO:400; or nucleotides 54-58 of SEQ ID NO:400, optionally wherein the short-sgRNA comprises modifications at least H1-1 to H1-5 and H2-1 to H2-12.
Embodiment 26 is the gRNA of any one of the preceding embodiments, wherein the at least 5-10 nucleotides:
   i. consist of 5-10 nucleotides;
   ii. consist of 6-10 nucleotides;
   iii. consist of 5 nucleotides;
   iv. consist of 6 nucleotides;
   v. consist of 7 nucleotides;
   vi. consist of 8 nucleotides;
   vii. consist of 9 nucleotides;
   viii. consist of 10 nucleotides;
   ix. consist of 5-10 consecutive nucleotides;
   x. consist of 6-10 consecutive nucleotides;
   xi. consist of 5 consecutive nucleotides;
   xii. consist of 6 consecutive nucleotides;
   xiii. consist of 7 consecutive nucleotides;
   xiv. consist of 8 consecutive nucleotides;
   xv. consist of 9 consecutive nucleotides; or
   xvi. consist of 10 consecutive nucleotides.
Embodiment 27 is the gRNA of any one of the preceding embodiments, wherein the 3′ end modification comprises one or more of:
   i. a phosphorothioate (PS) linkage between nucleotides;
   ii. a 2′-OMe modified nucleotide;
   iii. a 2′-O-moe modified nucleotide;
   iv. a 2′-F modified nucleotide;
   v. an inverted abasic modified nucleotide; and
   vi. a combination of one or more of (i.)-(v.).
Embodiment 28 is the gRNA of any one of the preceding embodiments, wherein the short-sgRNA comprises a 3′ tail comprising one or more of:
   i. a phosphorothioate (PS) linkage between nucleotides;
   ii. a 2′-OMe modified nucleotide;
   iii. a 2′-O-moe modified nucleotide;
   iv. a 2′-F modified nucleotide;
   v. an inverted abasic modified nucleotide; and
   vi. a combination of one or more of (i.)-(v.).
Embodiment 29 is the gRNA any one of the preceding embodiments, wherein the short-sgRNA comprises one or more of:
   i. 1, 2, 3, 4, 5, 6, 7, 8, 9, or 10 PS linkages between nucleotides;
   ii. 1, 2, 3, 4, 5, 6, 7, 8, 9, 10, 12, 14, 16, or 18 PS linkages between nucleotides;
   iii. about 1-3, 1-5, 1-6, 1-7, 1-8, 1-9, or 1-10 PS linkages between nucleotides;
   iv. about 1-3, 1-5, 1-6, 1-7, 1-8, 1-9, 1-10, 1-12, 1-14, 1-16, 1-18, or 1-20 PS linkages between nucleotides; and
   v. PS linkages between each nucleotide.
Embodiment 30 is the gRNA of any one of the preceding embodiments, wherein the 3′ end modification comprises at least one PS linkage, and wherein one or more of:
   i. there is one PS linkage, and the linkage is between the last and second to last nucleotide;
   ii. there are two PS linkages between the last three nucleotides;
   iii. there are PS linkages between any one or more of the last four nucleotides;
   iv. there are PS linkages between any one or more of the last five nucleotides; and
   v. there are PS linkages between any one or more of the last 2, 3, 4, 5, 6, 7, 8, 9, or 10 nucleotides.
Embodiment 31 is the gRNA of embodiment 31, wherein the 3′ end modification further comprises at least one 2′-OMe, 2′-O-moe, inverted abasic, or 2′-F modified nucleotide.
Embodiment 32 is the gRNA of any one of the preceding embodiments, wherein the 3′ end modification comprises:
   i. a modification of one or more of the last 1-7 nucleotides, wherein the modification is a PS linkage, inverted abasic nucleotide, 2′-OMe, 2′-O-moe, 2′-F, or combinations thereof;
   ii. a modification to the last nucleotide with 2′-OMe, 2′-O-moe, 2′-F, or combinations thereof, and an optional one or two PS linkages to the next nucleotide and/or the first nucleotide of the 3′ tail;
   iii. a modification to the last and/or second to last nucleotide with 2′-OMe, 2′-O-moe, 2′-F, or combinations thereof, and optionally one or more PS linkages;
   iv. a modification to the last, second to last, and/or third to last nucleotides with 2′-OMe, 2′-O-moe, 2′-F, or combinations thereof, and optionally one or more PS linkages;
   v. a modification to the last, second to last, third to last, and/or fourth to last nucleotides with 2′-OMe, 2′-O-moe, 2′-F, or combinations thereof, and optionally one or more PS linkages; or
   vi. a modification to the last, second to last, third to last, fourth to last, and/or fifth to last nucleotides with 2′-OMe, 2′-O-moe, 2′-F, or combinations thereof, and optionally one or more PS linkages.
Embodiment 33 is the gRNA of any one of the preceding embodiments, wherein the sgRNA comprise a 3′ tail, wherein the 3′ tail comprises a modification of any one or more of the nucleotides present in the 3′ tail.
Embodiment 34 is the gRNA of embodiment 33, wherein the 3′ tail is fully modified.
Embodiment 35 is the gRNA of embodiment 33, wherein the at least 5-10 nucleotides comprise nucleotides 54-61 of SEQ ID NO:400, nucleotides 53-60 of SEQ ID NO:400; or nucleotides 54-58 of SEQ ID NO:400, optionally wherein the short-sgRNA comprises modifications at least H1-1 to H1-5 and H2-1 to H2-12.
Embodiment 36 is the gRNA of any one of the preceding embodiments, wherein the short-sgRNA comprises any one or more of:
   i. the 3′ end modification as shown in any one of SEQ ID Nos: 1-54;
   ii. (i) a 2′-OMe modified nucleotide at the last nucleotide of the conserved region of an sgRNA or short-sgRNA, (ii) three consecutive 2′O-moe modified nucleotides immediately 5′ to the 2′-OMe modified nucleotide, and (iii) three consecutive PS linkages between the last three nucleotides;
   iii. (i) five consecutive 2′-OMe modified nucleotides from the 3′ end of the 3′ terminus, and (ii) three PS linkages between the last three nucleotides;
   iv. an inverted abasic modified nucleotide at the last nucleotide of the conserved region of an sgRNA or short-sgRNA;
   v. (i) an inverted abasic modified nucleotide at the last nucleotide of the conserved region of an sgRNA or short-sgRNA, and (ii) three consecutive 2′-OMe modified nucleotides at the last three nucleotides of the conserved region of an sgRNA or short-sgRNA;
   vi. (i) 15 consecutive 2′-OMe modified nucleotides from the 3′ end of the 3′ terminus, (ii) five consecutive 2′-F modified nucleotides immediately 5′ to the 2′-OMe modified nucleotides, and (iii) three PS linkages between the last three nucleotides;
   vii. (i) alternating 2′-OMe modified nucleotides and 2′-F modified nucleotides at the last 20 nucleotides of the conserved region of an sgRNA or short-sgRNA, and (ii) three PS linkages between the last three nucleotides;
   viii. (i) two or three consecutive 2′-OMe modified nucleotides, and (ii) three PS linkages between the last three nucleotides;
   ix. one PS linkage between the last and next to last nucleotides; and
   x. 15 or 20 consecutive 2′-OMe modified nucleotides, and (ii) three PS linkages between the last three nucleotides.
Embodiment 37 is the gRNA of any one of the preceding embodiments, wherein the 5′ end modification comprises any one or more of:
   i. a modification of any one or more of nucleotides 1-7 of the guide region;
   ii. one modified nucleotide;
   iii. two modified nucleotides;
   iv. three modified nucleotides;
   v. four modified nucleotides;
   vi. five modified nucleotides;
   vii. six modified nucleotides; and
   viii. seven modified nucleotides.
Embodiment 38 is the gRNA of any one of the preceding embodiments, wherein the 5′ end modification comprises a modification of between 1 and 7, between 1 and 5, between 1 and 4, between 1 and 3, or between 1 and 2 nucleotides.
Embodiment 39 is the gRNA of any one of the preceding embodiments, wherein the at least 5-10 nucleotides:
   i. comprise nucleotides 54-61 of SEQ ID NO:400;
   ii. comprise nucleotides 53-60 of SEQ ID NO:400;
   iii. comprise nucleotides 54-58 of SEQ ID NO:400.
   iv. consist of nucleotides 54-61 of SEQ ID NO:400;
   v. consist of nucleotides 53-60 of SEQ ID NO:400; or
   vi. consist of nucleotides 54-58 of SEQ ID NO:400.
Embodiment 40 is the gRNA of any one of the preceding embodiments, wherein the 5′ end modification comprises one or more of:
   i. a phosphorothioate (PS) linkage between nucleotides;
   ii. a 2′-OMe modified nucleotide;
   iii. a 2′-O-moe modified nucleotide;
   iv. a 2′-F modified nucleotide;
   v. an inverted abasic modified nucleotide;
   vi. a deoxyribonucleotide;
   vii. an inosine; and
   viii. combinations of one or more of (i.)-(vii.).
Embodiment 41 is the gRNA any one of the preceding embodiments, wherein the 5′ end modification comprises:
   i. 1, 2, 3, 4, 5, 6, and/or 7 PS linkages between nucleotides; or
   ii. about 1-2, 1-3, 1-4, 1-5, 1-6, or 1-7 PS linkages between nucleotides.
Embodiment 42 is the gRNA of any one of the preceding embodiments, wherein the 5′ end modification comprises at least one PS linkage, and wherein:
   i. there is one PS linkage, and the linkage is between nucleotides 1 and 2 of the guide region;
   ii. there are two PS linkages, and the linkages are between nucleotides 1 and 2, and 2 and 3 of the guide region;
   iii. there are PS linkages between any one or more of nucleotides 1 and 2, 2 and 3, and 3 and 4 of the guide region;
   iv. there are PS linkages between any one or more of nucleotides 1 and 2, 2 and 3, 3 and 4, and 4 and 5 of the guide region;
   v. there are PS linkages between any one or more of nucleotides 1 and 2, 2 and 3, 3 and 4, 4 and 5, and 5 and 6 of the guide region;
   vi. there are PS linkages between any one or more of nucleotides 1 and 2, 2 and 3, 3 and 4, 4 and 5, 5 and 6, and 6 and 7 of the guide region; or
   vii. there are PS linkages between any one or more of nucleotides 1 and 2, 2 and 3, 3 and 4, 4 and 5, 5 and 6, 6 and 7, and 7 and 8 of the guide region.
Embodiment 43 is the gRNA of embodiment 42, wherein the 5′ end modification further comprises at least one 2′-OMe, 2′-O-moe, inverted abasic, or 2′-F modified nucleotide.
Embodiment 44 is the gRNA of any one of the preceding embodiments, wherein the short-sgRNA comprises:
   i. a modification of one or more of nucleotides 1-7 of the variable region, wherein the modification is a PS linkage, inverted abasic nucleotide, 2′-OMe, 2′-O-moe, 2′-F, 2′-H (a deoxyribonucleotide), an inosine, and/or combinations thereof;
   ii. a modification to the first nucleotide of the guide region with 2′-OMe, 2′-O-moe, 2′-F, 2′-H, an inosine, or combinations thereof, and an optional PS linkage to the next nucleotide;
   iii. a modification to the first and/or second nucleotide of the variable region with 2′-OMe, 2′-O-moe, 2′-F, 2′-H, an inosine, or combinations thereof, and optionally one or more PS linkages;
   iv. a modification to the first, second, and/or third nucleotides of the variable region with 2′-OMe, 2′-O-moe, 2′-F, 2′-H, an inosine, or combinations thereof, and optionally one or more PS linkages;
   v. a modification to the first, second, third, and/or fourth nucleotides of the variable region with 2′-OMe, 2′-O-moe, 2′-F, 2′-H, an inosine, or combinations thereof, and optionally one or more PS linkages; or
   vi. a modification to the first, second, third, fourth, and/or fifth nucleotides of the variable region with 2′-OMe, 2′-O-moe, 2′-F, 2′-H, an inosine, or combinations thereof, and optionally one or more PS linkages.
Embodiment 45 is the gRNA of any one of the preceding embodiments, wherein the short-sgRNA comprises any one or more of:
   i. a 5′ end modification as shown in any one of SEQ ID Nos: 1-54;
   ii. 2′-OMe modified nucleotides at nucleotides 1, 2, and 3 of the guide region;
   iii. 2′-OMe modified nucleotides at nucleotides 1, 2, and 3 of the guide region and PS linkages between nucleotides 1 and 2, 2 and 3, and 3 and 4 of the guide region;
   iv. 2′-OMe modified nucleotides at nucleotides 1, 2, 3, 4, and 5 of the guide region;
   v. 2′-OMe modified nucleotides at nucleotides 1, 2, 3, 4, and 5 of the guide region and PS linkages between nucleotides 1 and 2, 2 and 3, 3 and 4, 4 and 5, and 5 and 6 of the guide region;
   vi. 2′O-moe modified nucleotides at nucleotides 1, 2, and 3 of the guide region;
   vii. 2′O-moe modified nucleotides at nucleotides 1, 2, and 3 of the guide region and PS linkages between nucleotides 1 and 2, 2 and 3, and 3 and 4 of the guide region;
   viii. an inverted abasic modified nucleotide at nucleotide 1 of the guide region;
   ix. an inverted abasic modified nucleotide at nucleotide 1 of the guide region and 2′-OMe modified nucleotides at nucleotides 1, 2, and 3 of the guide region; and
   x. an inverted abasic modified nucleotide at nucleotide 1 of the guide region, 2′-OMe modified nucleotides at nucleotides 1, 2, and 3 of the guide region, and PS linkages between nucleotides 1 and 2, 2 and 3, 3 and 4, 4 and 5, and 5 and 6 of the variable region.
Embodiment 46 is the gRNA of any one of the preceding embodiments, wherein the upper stem region comprises at least one modification.
Embodiment 47 is the gRNA of any one of the preceding embodiments, wherein the upper stem modification comprises any one or more of:
   i. a modification to any one or more of US1-US12 in the upper stem region;
   ii. a modification of at least 1, 2, 3, 4, 5, 6, 7, 8, 9, 10, 11, or all 12 nucleotides in the upper stem region; and
   iii. a modification of about 1-2, 1-3, 1-4, 1-5, 1-6, 1-7, 1-8, 1-9, 1-10, or 1-12 nucleotides in the upper stem region.
Embodiment 48 is the gRNA of embodiment 47, wherein the upper stem modification comprises one or more of:
   i. a 2′-OMe modified nucleotide;
   ii. a 2′-O-moe modified nucleotide;
   iii. a 2′-F modified nucleotide; and
   iv. combinations of one or more of (i.)-(iii.).
Embodiment 49 is a guide RNA which is a short-sgRNA comprising a conserved portion of an sgRNA comprising a hairpin region, wherein the hairpin region lacks at least 5-10 nucleotides and wherein the short-sgRNA comprises a 5′ end modification and one or more modification in one or more of:
   i. the upper stem region;
   ii. the hairpin 1 region; and
   iii. the hairpin 2 region,
      wherein the 5′ end modification comprises a 5′ protective end modification, such as at least two phosphorothioate (PS) linkages within the first seven nucleotides.
Embodiment 50 is the gRNA of embodiment 49, wherein at least one modification comprises a 2′-O-methyl (2′-OMe) modified nucleotide.
Embodiment 51 is the gRNA of embodiment 49 or embodiment 50, wherein at least one modification comprises a 2′-fluoro (2′-F) modified nucleotide.
Embodiment 52 is the gRNA of any one of embodiments 49-51, wherein at least one modification comprises a phosphorothioate (PS) bond between nucleotides.
Embodiment 53 is the gRNA of any one of embodiments 49-52, wherein the short-sgRNA comprises one or more modifications in the upper stem region.
Embodiment 54 is the gRNA of embodiment 53, comprising modifications at any one of US1 to US12.
Embodiment 55 is the gRNA of any one of embodiments 49-54, wherein the short-sgRNA comprises one or more modifications in the hairpin 1 region.
Embodiment 56 is the gRNA of embodiment 55, wherein the short-sgRNA comprises a modification at H1-1.
Embodiment 57 is the gRNA of any one of embodiments 49-56, wherein the short-sgRNA comprises one or more modifications in the hairpin 2 region.
Embodiment 58 is the gRNA of embodiment 57, wherein the short-sgRNA comprises a modification at H2-1.
Embodiment 59 is the gRNA of any one of embodiments 49-58, wherein the short-sgRNA comprises modifications at H1-1 to H1-12.
Embodiment 60 is the gRNA of any one of embodiments 49-59, wherein the short-sgRNA comprises modifications at H2-1 to H2-15.
Embodiment 61 is the gRNA of any one of embodiments 49-60, wherein the short-sgRNA comprises one or more modifications in each of the upper stem region, the hairpin 1 region, and the hairpin 2 region.
Embodiment 62 is the gRNA of any one of embodiments 49-61, wherein the short-sgRNA comprises a modified nucleotide between hairpin 1 and hairpin 2 regions.
Embodiment 63 is the gRNA of any one of embodiments 49-62, further comprising a lower stem region comprising a modification.
Embodiment 64 is the gRNA of any one of embodiments 49-63, further comprising a 3′ end modification.
Embodiment 65 is the gRNA of embodiment 64, wherein at least two of the last four nucleotides at the 3′ end of the 3′ terminus are modified.
Embodiment 66 is the gRNA of embodiment 64, wherein at least two of the last four nucleotides at the 3′ end of the 3′ terminus are modified with 2′-OMe, 2′-F, or 2′-O-moe.
Embodiment 67 is the gRNA of any one of embodiments 64-66, further comprising phosphorothioate (PS) bonds between one or more of the last four nucleotides at the 3′ end of the 3′ terminus.
Embodiment 68 is the gRNA of any one of embodiments 49-67, further comprising a bulge region comprising a modification.
Embodiment 69 is the gRNA of any one of embodiments 49-68, further comprising a nexus region comprising a modification.
Embodiment 70 is the gRNA of any one of embodiments 49-69, wherein at least the first three nucleotides at the 5′ end of the variable region, and the last three nucleotides at the 3′ end of the 3′ terminus are modified.
Embodiment 71 is the gRNA of any one of embodiments 49-70, wherein the first four nucleotides at the 5′ end of the variable region, and the last four nucleotides at the 3′ end of the 3′ terminus are linked with phosphorothioate (PS) bonds.
Embodiment 72 is the gRNA of any one of embodiments 70-71, wherein the end modifications comprise 2′-OMe.
Embodiment 73 is the gRNA of any one of embodiments 70-71, wherein the end modifications comprise 2′-F.
Embodiment 74 is the gRNA of any one of embodiments 49-73, wherein the first four nucleotides at the 5′ end of the variable region and the last four nucleotides at the 3′ end of the 3′ terminus are linked with a PS bond, and wherein the first three nucleotides at the 5′ end of the variable region and the last three nucleotides at the 3′ end of the 3′ terminus comprise 2′-OMe modifications.
Embodiment 75 is the gRNA of any one of embodiments 49-74, wherein the first four nucleotides at the 5′ terminus and the last four nucleotides at the 3′ terminus are linked with a PS bond, and wherein the first three nucleotides at the 5′ terminus and the last three nucleotides at the 3′ terminus comprise 2′-OMe, 2′-F, and/or 2′-O-moe modifications.
Embodiment 76 is the gRNA of any one of embodiments 49-75, wherein LS1, LS6, LS7, LS8, LS11, and/or LS12 are modified with 2′-OMe.
Embodiment 77 is the gRNA of any one of embodiments 49-76, wherein each of the nucleotides in the bulge region are modified with 2′-OMe.
Embodiment 78 is the gRNA of any one of embodiments 49-77, wherein at least 50% of the nucleotides in the bulge region are modified with 2′-OMe.
Embodiment 79 is the gRNA of any one of embodiments 49-78, wherein each of the nucleotides in the upper stem region are modified with 2′-OMe.
Embodiment 80 is the gRNA of any one of embodiments 49-79, wherein N16, N17, and/or N18 in the nexus region are modified with 2′-OMe.
Embodiment 81 is the gRNA of any one of embodiments 49-80, wherein N15, N16, N17, and/or N18 in the nexus region are modified.
Embodiment 82 is the gRNA of embodiment 80 or 81, wherein the modifications in the nexus region are selected from 2′-OMe and 2′F.
Embodiment 83 is the gRNA of any one of embodiments 80-82, wherein N16, N17, and N18 are linked with PS bonds.
Embodiment 84 is the gRNA of any one of embodiments 49-83, wherein each of the nucleotides remaining in the hairpin 1 region are modified with 2′-OMe.
Embodiment 85 is the gRNA of any one of embodiments 49-84, wherein each of the nucleotides in the hairpin 2 region are modified with 2′-OMe.
Embodiment 86 is a guide RNA which is a short-sgRNA comprising a conserved portion of an sgRNA comprising a hairpin region, wherein the hairpin region lacks at least 5-10 nucleotides and wherein the short-sgRNA comprises a 5′ end modification and a 3′ end modification, wherein the short-sgRNA further comprises any one or more of:
   i. at least one modification in the upper stem region; and
   ii. a 3′ tail.
Embodiment 87 is the gRNA of embodiment 86, wherein the upper stem modification comprises any one or more of:
   i. a modification of each nucleotide (US1-US12) in the upper stem region;
   ii. a modification to any one or more of US1-US12 in the upper stem region;
   iii. a modification of at least 1, 2, 3, 4, 5, 6, 7, 8, 9, 10, 11, or all 12 nucleotides in the upper stem region; and
   iv. a modification of about 1-2, 1-3, 1-4, 1-5, 1-6, 1-7, 1-8, 1-9, 1-10, or 1-12 nucleotides in the upper stem region.
Embodiment 88 is the gRNA of any one of embodiments 86-87, wherein the 5′ end modification comprises any one or more of:
   i. a modification of any one or more of nucleotides 1-7 of the variable region;
   ii. one modified nucleotide;
   iii. two modified nucleotides;
   iv. three modified nucleotides;
   v. four modified nucleotides;
   vi. five modified nucleotides;
   vii. six modified nucleotides; and
   viii. seven modified nucleotides.
Embodiment 89 is the gRNA of any one of embodiments 86-88, wherein the 5′ end modification comprises any one or more of:
   i. a 5′ end modification as shown in any one of SEQ ID Nos: 1-54, 401-532, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, 3388-3430, or 3549-3552;
   ii. 2′-OMe modified nucleotides at nucleotides 1, 2, and 3 of the variable region;
   iii. 2′-OMe modified nucleotides at nucleotides 1, 2, and 3 of the variable region and PS linkages between nucleotides 1 and 2, 2 and 3, and 3 and 4 of the variable region;
   iv. 2′-OMe modified nucleotides at nucleotides 1, 2, 3, 4, and 5 of the variable region;
   v. 2′-OMe modified nucleotides at nucleotides 1, 2, 3, 4, and 5 of the variable region and PS linkages between nucleotides 1 and 2, 2 and 3, 3 and 4, 4 and 5, and 5 and 6 of the variable region;
   vi. 2′O-moe modified nucleotides at nucleotides 1, 2, and 3 of the variable region;
   vii. 2′O-moe modified nucleotides at nucleotides 1, 2, and 3 of the variable region and PS linkages between nucleotides 1 and 2, 2 and 3, and 3 and 4 of the variable region;
   viii. an inverted abasic modified nucleotide at nucleotide 1 of the variable region;
   ix. an inverted abasic modified nucleotide at nucleotide 1 of the variable region and 2′-OMe modified nucleotides at nucleotides 1, 2, and 3 of the variable region; and
   x. an inverted abasic modified nucleotide at nucleotide 1 of the variable region, 2′-OMe modified nucleotides at nucleotides 1, 2, and 3 of the variable region, and PS linkages between nucleotides 1 and 2, 2 and 3, 3 and 4, 4 and 5, and 5 and 6 of the variable region.
Embodiment 90 is the gRNA of embodiment 89, comprising 2′-OMe modified nucleotides at at least nucleotides 1, 2, and 3, of the variable region and PS linkages between nucleotides 1 and 2, 2 and 3, and 3 and 4 of the variable region.
Embodiment 91 is the gRNA of embodiment 89, comprising 2′-OMe modified nucleotides at at least nucleotides 1, 2, 3, and 4 of the variable region and PS linkages between nucleotides 1 and 2, 2 and 3, and 3 and 4 of the variable region.
Embodiment 92 is the gRNA of any one of embodiments 86-91, comprising a 3′ end modification comprising any one or more of:
   i. a modification of any one or more of the last 7, 6, 5, 4, 3, 2, or 1 nucleotides;
   ii. one modified nucleotide;
   iii. two modified nucleotides;
   iv. three modified nucleotides;
   v. four modified nucleotides;
   vi. five modified nucleotides;
   vii. six modified nucleotides; and
   viii. seven modified nucleotides.
Embodiment 93 is the gRNA of any one of embodiments 86-92, wherein the short-sgRNA comprises any one or more of:
   i. a 3′ end modification shown in any one of SEQ ID Nos: 1-54, 401-532, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, 3388-3430, or 3549-3552;
   ii. (i) a 2′-OMe modified nucleotide at the last nucleotide of the conserved region of an sgRNA or short-sgRNA, (ii) three consecutive 2′O-moe modified nucleotides immediately 5′ to the 2′-OMe modified nucleotide, and (iii) three consecutive PS linkages between the last three nucleotides;
   iii. (i) five consecutive 2′-OMe modified nucleotides, and (ii) three PS linkages between the last three nucleotides;
   iv. an inverted abasic modified nucleotide at the last nucleotide of the conserved region of an sgRNA or short-sgRNA;
   v. (i) an inverted abasic modified nucleotide at the last nucleotide of the conserved region of an sgRNA or short-sgRNA, and (ii) three consecutive 2′-OMe modified nucleotides at the last three nucleotides of the conserved region of an sgRNA or short-sgRNA;
   vi. (i) 15 consecutive 2′-OMe modified nucleotides, (ii) five consecutive 2′-F modified nucleotides immediately 5′ to the 2′-OMe modified nucleotides, and (iii) three PS linkages between the last three nucleotides;
   vii. (i) alternating 2′-OMe modified nucleotides and 2′-F modified nucleotides at the last 20 nucleotides of the conserved region of an sgRNA or short-sgRNA, and (ii) three PS linkages between the last three nucleotides;
   viii. (i) two or three consecutive 2′-OMe modified nucleotides, and (ii) three PS linkages between the last three nucleotides;
   ix. one PS linkage between the last and next to last nucleotides; and
   x. 15 or 20 consecutive 2′-OMe modified nucleotides, and (ii) three PS linkages between the last three nucleotides.
Embodiment 94 is the gRNA of any one of embodiments 86-93, wherein the sgRNA comprise a 3′ tail, wherein the 3′ tail comprises a modification of any one or more of the nucleotides present in the 3′ tail.
Embodiment 95 is the gRNA of embodiment 94, wherein the 3′ tail is fully modified.
Embodiment 96 is the gRNA of embodiment 94 wherein the 3′ tail comprises 1, 2, 3, 4, 5, 6, 7, 8, 9, 10, 1-2, 1-3, 1-4, 1-5, 1-6, 1-7, 1-8, 1-9, or 1-10 nucleotides, optionally where any one or more of these nucleotides are modified.
Embodiment 97 is a guide RNA which is a short-sgRNA comprising any of SEQ ID Nos: 1-54, 201-254, and 301-354, including the modifications of Table 1.
Embodiment 98 is a guide RNA which is a short-sgRNA comprising nucleic acids having at least 99, 98, 97, 96, 95, 94, 93, 92, 91, 90, 85, 80, 75, or 70% identity to the nucleic acids of any one of SEQ ID Nos: 1-54, 201-254, and 301-354, wherein the modification at each nucleotide of the short-sgRNA that corresponds to a nucleotide of the reference sequence identifier in Table 1, is identical to or equivalent to the modification shown in the reference sequence identifier in Table 1.
Embodiment 99 is the gRNA of any one of the preceding embodiments, comprising a YA modification at at least one guide region YA site.
Embodiment 100 is the gRNA of any one of the preceding embodiments, comprising a YA modification at at least one guide region YA site that is not a 5′ end modification.
Embodiment 101 is the gRNA of any one of the preceding embodiments, comprising a YA modification at one or more guide region YA sites, wherein the guide region YA site is at or after nucleotide 8 from the 5′ end of the 5′ terminus.
Embodiment 102 is the gRNA of any one of the preceding embodiments comprising a YA modification at one or more guide region YA sites, wherein the short-sgRNA comprises one or more of:
   a. a modification at one or more of H1-1 and H2-1;
   b. a YA modification at 1, 2, 3, 4, or 5 guide region YA sites;
   c. a YA modification at 1, 2, 3, 4, or 5 guide region YA sites, wherein the modification of at least one guide region YA site is different from any 5′ end modification of the sgRNA;
   d. a YA modification at one or more guide region YA sites, wherein the guide region YA site is at or after nucleotide 8 from the 5′ end of the 5′ terminus;
   e. a YA modification at one or more guide region YA sites, wherein the guide region YA site is within nucleotides 5-end, 6-end, 7-end, 8-end, 9-end, or 10-end from the 5′ end of the 5′ terminus;
   f. a YA modification at one or more guide region YA sites, wherein the guide region YA site is within 17, 16, 15, 14, 13, 12, 11, 10, or 9 nucleotides of the 3′ terminal nucleotide of the guide region;
   g. a YA modification at a guide region YA site other than a 5′ end modification;
   h. a YA modification at two or more guide region YA sites, wherein the guide region YA sites are at or after nucleotide 8 from the 5′ end of the 5′ terminus;
   i. a YA modification at two or more guide region YA sites, wherein the two guide region YA sites are within nucleotides 5-end, 6-end, 7-end, 8-end, 9-end, or 10-end from the 5′ end of the 5′ terminus;
   j. a YA modification at two or more guide region YA sites, wherein the guide region YA sites are within 17, 16, 15, 14, 13, 12, 11, 10, or 9 nucleotides of the 3′ terminal nucleotide of the guide region;
   k. a YA modification at two or more guide region YA sites other than a 5′ end modification; and
   l. a YA modification at two or more guide region YA sites, wherein the modifications of the guide region YA sites comprise a modification that at least one nucleotide located 5′ of the guide region YA site does not comprise.
Embodiment 103 is the gRNA of any one of the preceding embodiments, comprising a YA modification wherein the modification comprises 2′-fluoro, 2′-H, 2′-OMe, ENA, UNA, inosine, or PS.
Embodiment 104 is the gRNA of any one of the preceding embodiments, comprising a YA modification wherein the modification alters the structure of the dinucleotide motif to reduce RNA endonuclease activity.
Embodiment 105 is the gRNA of any one of the preceding embodiments, comprising a YA modification wherein the modification interferes with recognition or cleavage of a YA site by an RNase and/or stabilizes an RNA structure.
Embodiment 106 is the gRNA of any one of the preceding embodiments, comprising a YA modification wherein the modification comprises one or more of:
   a. a ribose modification selected from 2′-O-alkyl, 2′-F, 2′-moe, 2′-F arabinose, and 2′-H (deoxyribose);
   b. a bicyclic ribose analog, such as LNA, BNA, and ENA;
   c. an unlocked nucleic acid modification;
   d. a base modification, such as inosine, pseudouridine, and 5′-methylcytosine; and
   e. an internucleoside linkage modification such as phosphorothioate.
Embodiment 107 is the gRNA of any one of the preceding embodiments, comprising a YA modification at one or more conserved region YA sites.
Embodiment 108 is the gRNA of any one of the preceding embodiments, comprising a YA modification at one or more of conserved region YA sites 2, 3, 4, and 10.
Embodiment 109 is the gRNA of any one of the preceding embodiments, comprising a YA modification at one or more of conserved region YA sites 1 and 8.
Embodiment 110 is the gRNA of any one of the preceding embodiments, comprising a YA modification of conserved region YA site 1.
Embodiment 111 is the gRNA of any one of the preceding embodiments, comprising a YA modification of conserved region YA site 2.
Embodiment 112 is the gRNA of any one of the preceding embodiments, comprising a YA modification of conserved region YA site 3.
Embodiment 113 is the gRNA of any one of the preceding embodiments, comprising a YA modification of conserved region YA site 4.
Embodiment 114 is the gRNA of any one of the preceding embodiments, comprising a YA modification of conserved region YA site 5.
Embodiment 115 is the gRNA of any one of the preceding embodiments, comprising a YA modification of conserved region YA site 6.
Embodiment 116 is the gRNA of any one of the preceding embodiments, comprising a YA modification of conserved region YA site 7.
Embodiment 117 is the gRNA of any one of the preceding embodiments, comprising a YA modification of conserved region YA site 8.
Embodiment 118 is the gRNA of any one of the preceding embodiments, comprising a YA modification of conserved region YA site 9.
Embodiment 119 is the gRNA of any one of the preceding embodiments, comprising a YA modification of conserved region YA site 10.
Embodiment 120 is the gRNA of any one of the preceding embodiments, comprising one or more of:
   a. YA modifications of conserved region YA sites 2, 3, 4, and 10;
   b. YA modifications of conserved region YA sites 2, 3, and 4;
   c. YA modifications of conserved region YA sites 2, 3, and 10;
   d. YA modifications of conserved region YA sites 2, 4, and 10;
   e. YA modifications of conserved region YA sites 3, 4, and 10;
   f. YA modifications of conserved region YA sites 2 and 10;
   g. YA modifications of conserved region YA sites 2 and 4;
   h. YA modifications of conserved region YA sites 2 and 3;
   i. YA modifications of conserved region YA sites 3 and 4;
   j. YA modifications of conserved region YA sites 3 and 10;
   k. YA modifications of conserved region YA sites 4 and 10
   l. YA modifications of conserved region YA sites 1 and 5;
   m. YA modifications of conserved region YA sites 1 and 6;
   n. YA modifications of conserved region YA sites 1 and 7;
   o. YA modifications of conserved region YA sites 1 and 8;
   p. YA modifications of conserved region YA sites 1 and 9;
   q. YA modifications of conserved region YA sites 8 and 5;
   r. YA modifications of conserved region YA sites 8 and 6;
   s. YA modifications of conserved region YA sites 8 and 7; and
   t. YA modifications of conserved region YA sites 8 and 9;
      optionally wherein the sgRNA further comprises YA modifications of conserved region YA sites 2, 3, 4, and/or 10.
Embodiment 121 is the gRNA of any one of the preceding embodiments, wherein at least one modified YA site comprises a 2′-OMe modification, optionally at the pyrimidine of the YA site.
Embodiment 122 is the gRNA of any one of the preceding embodiments, wherein at least one modified YA site comprises a 2′-fluoro modification, optionally at the pyrimidine of the YA site.
Embodiment 123 is the gRNA of any one of the preceding embodiments, wherein at least one modified YA site comprises a PS modification, optionally at the pyrimidine of the YA site.
Embodiment 124 is the gRNA of any one of the preceding embodiments, wherein the gRNA comprises a guide region that comprises modifications at at least 1, 2, 3, 4, 5, 6, 7, 8, 9, 10, 11, 12, 13, or all of the following nucleotides: 1, 2, 3, 4, 6, 7, 8, 9, 10, 11, 13, 14, 17, and 18, optionally wherein the modifications are 2′-OMe, 2′-fluoro, 2′-H, inosine, or phosphorothioate modifications.
Embodiment 125 is the gRNA of any one of the preceding embodiments, wherein the short-sgRNA comprises a guide region that comprises modifications at nucleotides 1, 2, 3, 4, 6, 7, 8, 9, 10, 11, 13, 14, 17, and 18, optionally wherein the modifications are 2′-OMe, 2′-fluoro, 2′-H, inosine, or phosphorothioate modifications.
Embodiment 126 is the gRNA of embodiments 124-125, wherein 2′-OMe modifications are not present in the guide region at nucleotides 6-11 and 13-end.
Embodiment 127 is the gRNA of embodiments 124-126, wherein 2′-fluoro modifications are not present in the guide region at nucleotides 1-7, 15, 16, and 19-end.
Embodiment 128 is the gRNA of embodiments 124-127, wherein phosphorothioate modifications are not present in the guide region at nucleotides 4, 5, 11-14, 17, and 18.
Embodiment 129 is the gRNA of embodiments 124-128, wherein the guide region comprises an unmodified nucleotide 20.
Embodiment 130 is the gRNA of embodiments 124-129, wherein the guide region consists of 20 nucleotides.
Embodiment 131 is the gRNA of embodiments 124-130, wherein the guide region comprises a YA site at nucleotides 5-6 and a modification at nucleotide 5.
Embodiment 132 is the gRNA of embodiments 124-131, wherein the guide region comprises a YA site at nucleotides 12-13 and a modification at nucleotide 12.
Embodiment 133 is the gRNA of embodiments 124-132, wherein the guide region comprises a YA site at nucleotides 15-16 and a modification at nucleotide 15.
Embodiment 134 is the gRNA of embodiments 124-133, wherein the guide region comprises a YA site at nucleotides 16-17 and a modification at nucleotide 16.
Embodiment 135 is the gRNA of embodiments 124-134, wherein the guide region comprises a YA site at nucleotides 19-20 and a modification at nucleotide 19.
Embodiment 136 is the gRNA of embodiments 124-130 or 132-135, wherein the guide region does not comprise a YA site at nucleotides 5-6 and nucleotide 5 is unmodified.
Embodiment 137 is the gRNA of embodiments 124-131 or 133-136, wherein the guide region does not comprise a YA site at nucleotides 12-13 and nucleotide 12 is unmodified.
Embodiment 138 is the gRNA of embodiments 124-132 or 134-137, wherein the guide region does not comprise a YA site at nucleotides 15-16 and nucleotide 15 is unmodified.
Embodiment 139 is the gRNA of embodiments 124-133 or 135-138, wherein the guide region does not comprise a YA site at nucleotides 16-17 and nucleotide 16 is unmodified.
Embodiment 140 is the gRNA of embodiments 124-134 or 136-139, wherein the guide region does not comprise a YA site at nucleotides 19-20 and nucleotide 19 is unmodified.
Embodiment 141 is the gRNA of embodiments 124-140, wherein the short-sgRNA comprises a guide region that comprises one or more of the following:
   (a) 2′-OMe and phosphorothioate modifications at nucleotide 1;
   (b) 2′-OMe and phosphorothioate modifications at nucleotide 2;
   (c) 2′-OMe and phosphorothioate modifications at nucleotide 3;
   (d) a 2′-OMe modification at nucleotide 4;
   (e) a phosphorothioate modification at nucleotide 6;
   (f) a phosphorothioate modification at nucleotide 7;
   (g) 2′-fluoro and phosphorothioate modifications at nucleotide 8;
   (h) 2′-fluoro and phosphorothioate modifications at nucleotide 9;
   (i) 2′-fluoro and phosphorothioate modifications at nucleotide 10;
   (j) a 2′-fluoro modification at nucleotide 11;
   (k) a 2′-fluoro modifications at nucleotide 13;
   (l) a 2′-fluoro modifications at nucleotide 14;
   (m)a 2′-fluoro modifications at nucleotide 17; and
   (n) a 2′-fluoro modifications at nucleotide 18.
Embodiment 142 is the gRNA of embodiments 124-141, wherein the guide region comprises each of the modifications set forth in the preceding embodiment.
Embodiment 143 is the gRNA of embodiments 124-142, wherein the guide region comprises at least 1, 2, 3, or 4 of the following:
   (a) a 2′-OMe modification at nucleotide 5 if nucleotides 5 and 6 form a YA site;
   (b) a 2′-OMe modification at nucleotide 12 if nucleotides 12 and 13 form a YA site;
   (c) a phosphorothioate or 2′-H modification at nucleotide 15 if nucleotides 15 and 16 form a YA site;
   (d) a phosphorothioate modification at nucleotide 16 if nucleotides 16 and 17 form a YA site; and
   (e) a phosphorothioate or 2′-fluoro modification at nucleotide 19 if nucleotides 19 and 20 form a YA site.
Embodiment 144 is the gRNA of embodiments 124-143, wherein the guide region comprises a YA site at nucleotides 5-6 and a 2′-OMe modification at nucleotide 5.
Embodiment 145 is the gRNA of embodiments 124-144, wherein the guide region comprises a YA site at nucleotides 12-13 and a 2′-OMe modification at nucleotide 12.
Embodiment 146 is the gRNA of embodiments 124-145, wherein the guide region comprises a YA site at nucleotides 15-16 and a phosphorothioate modification at nucleotide 15.
Embodiment 147 is the gRNA of embodiments 124-146, wherein the guide region comprises a YA site at nucleotides 16-17 and a phosphorothioate modification at nucleotide 16.
Embodiment 148 is the gRNA of embodiments 124-147, wherein the guide region comprises a YA site at nucleotides 19-20 and a phosphorothioate modification at nucleotide 19.
Embodiment 149 is the gRNA of embodiments 124-148, wherein the guide region comprises a 2′-fluoro modification at nucleotide 19.
Embodiment 150 is the gRNA of embodiments 124-149, wherein the guide region comprises an unmodified nucleotide 15 or only a phosphorothioate modification at nucleotide 15.
Embodiment 151 is the gRNA of embodiments 124-150, wherein the guide region comprises an unmodified nucleotide 16 or only a phosphorothioate modification at nucleotide 16.
Embodiment 152 is a guide RNA which is a single guide RNA (sgRNA) comprising:
   a. a YA modification at two or more guide region YA sites;
   b. a YA modification at one or more of conserved region YA sites 2, 3, 4, and 10; and
   c. a YA modification at one or more of conserved region YA sites 1 and 8.
Embodiment 153 is a guide RNA which is a single guide RNA (sgRNA) comprising:
   a. a YA modification at one or more guide region YA sites that are at or after nucleotide 8 from the 5′ end of the 5′ terminus;
   b. a YA modification at one or more of conserved region YA sites 2, 3, 4, and 10; and optionally
   c. a YA modification at one or more of conserved region YA sites 1 and 8.
Embodiment 154 is a guide RNA which is a single guide RNA (sgRNA) comprising:
   a. a YA modification at one or more guide region YA sites that are within 13 nucleotides of the 3′ terminal nucleotide of the guide region;
   b. a YA modification at one or more of conserved region YA sites 2, 3, 4, and 10; and
   c. a YA modification at one or more of conserved region YA sites 1 and 8.
Embodiment 155 is a guide RNA which is a single guide RNA (sgRNA) comprising:
   a. a 5′ end modification and a 3′ end modification;
   b. a YA modification at one or more of conserved region YA sites 2, 3, 4, and 10; and
   c. a YA modification at one or more of conserved region YA sites 1 and 8.
Embodiment 156 is a guide RNA which is a single guide RNA (sgRNA) comprising:
   a. a YA modification at at least one guide region YA site, wherein the modification of the guide region YA site comprises a modification at at least one nucleotide located 5′ of the guide region YA site does not comprise;
   b. a YA modification at one or more of conserved region YA sites 2, 3, 4, and 10; and
   c. a YA modification at one or more of conserved region YA sites 1 and 8.
Embodiment 157 is a guide RNA which is a single guide RNA (sgRNA) comprising:
   a. a YA modification at one or more of conserved region YA sites 2, 3, 4, and 10; and
   b. a YA modification at conserved region YA sites 1 and 8.
Embodiment 158 is a guide RNA which is a single guide RNA (sgRNA) comprising:
   a. a YA modification at one or more guide region YA sites that are at or after nucleotide 8 from the 5′ end of the 5′ terminus;
   b. a YA modification at one or more of conserved region YA sites 2, 3, 4, and 10; and
   c. a modification at one or more of H1-1 and H2-1.
Embodiment 159 is a guide RNA which is a single guide RNA (sgRNA) comprising:
   a. a YA modification at one or more of conserved region YA sites 2, 3, 4, and 10;
   b. a YA modification at one or more of conserved region YA sites 1, 5, 6, 7, 8, and 9; and
   c. a modification at one or more of H1-1 and H2-1.
Embodiment 160 is a guide RNA which is an sgRNA comprising any one or more of the following:
   a. a modification, such as a YA modification, at one or more nucleotides located at or after nucleotide 6 from the 5′ end of the 5′ terminus;
   b. a YA modification at one or more guide sequence YA sites;
   c. a modification at one or more of B3, B4, and B5, wherein B6 does not comprise a 2′-OMe modification or comprises a modification other than 2′-OMe;
   d. a modification at LS10, wherein LS10 comprises a modification other than 2′-fluoro; and/or
   e. a modification at N2, N3, N4, N5, N6, N7, N10, or N11; and
      wherein at least one of the following is true:
   i. at least one of nucleotides 8-11, 13, 14, 17, or 18 from the 5′ end of the 5′ terminus does not comprise a 2′-fluoro modification;
   ii. at least one of nucleotides 6-10 from the 5′ end of the 5′ terminus does not comprise a phosphorothioate linkage;
   iii. at least one of B2, B3, B4, or B5 does not comprise a 2′-OMe modification;
   iv. at least one of LS1, LS8, or LS10 does not comprise a 2′-OMe modification;
   v. at least one of N2, N3, N4, N5, N6, N7, N10, N11, N16, or N17 does not comprise a 2′-OMe modification;
   vi. H1-1 comprises a modification;
   vii. H2-1 comprises a modification; or
   viii. at least one of H1-2, H1-3, H1-4, H1-5, H1-6, H1-7, H1-8, H1-9, H1-10, H2-1, H2-2, H2-3, H2-4, H2-5, H2-6, H2-7, H2-8, H2-9, H2-10, H2-11, H2-12, H2-13, H2-14, or H2-15 does not comprise a phosphorothioate linkage.
Embodiment 161 is a guide RNA comprising any one or more of the following:
   i. a modification, such as a YA modification, at one or more nucleotides located at or after nucleotide 6 from the 5′ end of the 5′ terminus; or
   ii. a YA modification at one or more guide sequence YA sites;
      wherein at least one of the following is true:
   a. at least one of nucleotides 8-11, 13, 14, 17, or 18 from the 5′ end of the 5′ terminus does not comprise a 2′-fluoro modification; or
   b. at least one of nucleotides 6-10 from the 5′ end of the 5′ terminus does not comprise a phosphorothioate linkage;
      and wherein at least one of the following is also true:
   c. at least one of nucleotides 7-10 from the 5′ end of the 5′ terminus does not comprise a 2′-OMe modification;
   d. nucleotide 20 from the 5′ end of the 5′ terminus does not comprise a 2′-OMe modification; or
   e. the guide RNA comprises a 2′-fluoro modification at any one or more of nucleotides 1-20 from the 5′ end of the 5′ terminus and at least one of nucleotides 11, 12, 13, 14, 17, or 18 from the 5′ end of the 5′ terminus does not comprise a 2′-fluoro modification, optionally wherein nucleotide 12 from the 5′ end of the 5′ terminus does not comprise a 2′-fluoro modification.
Embodiment 162 is a guide RNA which is an sgRNA comprising a guanosine at N14 and/or one or more of the following:
   a. a YA modification at one or more guide region YA sites that are at or after nucleotide 8 from the 5′ end of the 5′ terminus;
   b. a YA modification at one or more of conserved region YA sites 1, 5, and 6, wherein if YA site 6 is modified at LS12 and LS9is not modified, then the modification of LS12 is other than 2′-OMe;
   c. a modification at LS9, wherein if LS9 is modified and LS5, LS7, and LS12 are not modified, then the modification of LS9 is other than 2′-fluoro,
   d. a modification at LS12, wherein if LS12 is modified and LS9 is not modified, then the modification of LS12 is other than 2′-OMe;
   e. a modification at LS8 or LS11, wherein at least one of LS8 and LS11 comprises a modification other than 2′-OMe; and/or
   f a modification at N6, N14, or N17, wherein if N17 is modified and N6 and N14 are not modified, then the modification of N17 is other than 2′-fluoro and other than 2′-OMe;
      and wherein at least one of the following is true:
   i. at least one of nucleotides 8-11, 13-14, 17, or 18 from the 5′ end of the 5′ terminus does not comprise a 2′-fluoro modification;
   ii. at least one of nucleotides 6-10 from the 5′ end of the 5′ terminus does not comprise a phosphorothioate linkage;
   iii. at least one of B2, B3, B4, or B5 does not comprise a 2′-OMe modification;
   iv. at least one of LS1, LS8, or LS10 does not comprise a 2′-OMe modification;
   v. at least one of N2, N3, N4, N5, N6, N7, N10, N11, N16, or N17 does not comprise a 2′-OMe modification;
   vi. H1-1 comprises a modification;
   vii. H2-1 comprises a modification; or
   viii. at least one of H1-2, H1-3, H1-4, H1-5, H1-6, H1-7, H1-8, H1-9, H1-10, H2-1, H2-2, H2-3, H2-4, H2-5, H2-6, H2-7, H2-8, H2-9, H2-10, H2-11, H2-12, H2-13, H2-14, or H2-15 does not comprise a phosphorothioate linkage.
Embodiment 163 is the gRNA of embodiments 161 or 162, comprising:
   a. a YA modification at 1, 2, 3, 4, or 5 guide region YA sites;
   b. a YA modification at 1, 2, 3, 4, or 5 guide region YA sites, wherein the modification of at least one guide region YA site is different from any 5′ end modification of the sgRNA;
   c. a YA modification at one or more guide region YA sites that are at or after nucleotide 8 from the 5′ end of the 5′ terminus;
   d. a YA modification at one or more guide region YA sites that are is within nucleotides 5-end, 6-end, 7-end, 8-end, 9-end, or 10-end from the 5′ end of the 5′ terminus;
   e. a YA modification at one or more guide region YA sites that are within 17, 16, 15, 14, 13, 12, 11, 10, or 9 nucleotides of the 3′ terminal nucleotide of the guide region;
   f a YA modification at a guide region YA site other than a 5′ end modification; or
   g. a YA modification at a guide region YA site, wherein the modification of the guide region YA site comprises a modification at at least one nucleotide located 5′ of the guide region YA site does not comprise.
Embodiment 164 is the gRNA of embodiment 163, comprising:
   a. a YA modification at two or more guide region YA sites that are at or after nucleotide 8 from the 5′ end of the 5′ terminus;
   b. a YA modification at two or more guide region YA sites that are within nucleotides 5-end, 6-end, 7-end, 8-end, 9-end, or 10-end from the 5′ end of the 5′ terminus;
   c. a YA modification at two or more guide region YA sites that are within 17, 16, 15, 14, 13, 12, 11, 10, or 9 nucleotides of the 3′ terminal nucleotide of the guide region;
   d. a YA modification at two or more guide region YA sites other than a 5′ end modification; or
   e. a YA modification at a two or more guide region YA sites, wherein the modifications of the guide region YA sites comprise a modification at at least one nucleotide located 5′ of the guide region YA site does not comprise.
Embodiment 165 is the gRNA of embodiment 163, comprising:
   a. a YA modification at three or more guide region YA sites that are at or after nucleotide 8 from the 5′ end of the 5′ terminus;
   b. a YA modification at three or more guide region YA sites that are within nucleotides 5-end, 6-end, 7-end, 8-end, 9-end, or 10-end from the 5′ end of the 5′ terminus;
   c. a YA modification at three or more guide region YA sites that are within 17, 16, 15, 14, 13, 12, 11, 10, or 9 nucleotides of the 3′ terminal nucleotide of the guide region;
   d. a YA modification at three or more guide region YA sites other than a 5′ end modification; or
   e. a YA modification at a three or more guide region YA sites, wherein the modifications of the guide region YA sites comprise a modification at at least one nucleotide located 5′ of the guide region YA site does not comprise.
Embodiment 166 is the gRNA of any one of embodiments 161-165, comprising at least one YA modification at nucleotide 6 from the 5′ end of the 5′ terminus.
Embodiment 167 is the gRNA of any one of embodiments 161-166, comprising at least one YA modification at nucleotide 7 from the 5′ end of the 5′ terminus.
Embodiment 168 is the gRNA of any one of embodiments 161-167, comprising at least one YA modification at nucleotide 8 from the 5′ end of the 5′ terminus.
Embodiment 169 is the gRNA of any one of embodiments 161-168, comprising at least one YA modification at nucleotide 9 from the 5′ end of the 5′ terminus.
Embodiment 170 is the gRNA of any one of embodiments 161-169, comprising at least one YA modification at nucleotide 10 from the 5′ end of the 5′ terminus.
Embodiment 171 is the gRNA of any one of embodiments 161-170, comprising at least one YA modification at nucleotide 11 from the 5′ end of the 5′ terminus.
Embodiment 172 is the gRNA of any one of embodiments 161-171, comprising at least one YA modification at nucleotide 12 from the 5′ end of the 5′ terminus.
Embodiment 173 is the gRNA of any one of embodiments 161-172, comprising at least one YA modification at nucleotide 13 from the 5′ end of the 5′ terminus.
Embodiment 174 is the gRNA of any one of embodiments 161-173, comprising at least one YA modification at nucleotide 14 from the 5′ end of the 5′ terminus.
Embodiment 175 is the gRNA of any one of embodiments 161-174, comprising at least one YA modification at nucleotide 15 from the 5′ end of the 5′ terminus.
Embodiment 176 is the gRNA of any one of embodiments 161-175, comprising at least one YA modification at nucleotide 16 from the 5′ end of the 5′ terminus.
Embodiment 177 is the gRNA of any one of embodiments 161-176, comprising at least one YA modification at nucleotide 17 from the 5′ end of the 5′ terminus.
Embodiment 178 is the gRNA of any one of embodiments 161-177, comprising at least one YA modification at nucleotide 18 from the 5′ end of the 5′ terminus.
Embodiment 179 is the gRNA of any one of embodiments 161-178, comprising at least one YA modification at nucleotide 19 from the 5′ end of the 5′ terminus.
Embodiment 180 is the gRNA of any one of embodiments 161-179, comprising at least one YA modification at nucleotide 20 from the 5′ end of the 5′ terminus.
Embodiment 181 is the gRNA of any one of embodiments 161-180, wherein at least 1, 2, 3, 4, 5, 6, 7, or 8 of nucleotides 8-11, 13-14, and 17-18 from the 5′ end of the 5′ terminus comprise a YA modification, optionally wherein the modification comprises 2′-fluoro, 2′-H, 2′-OMe, or PS.
Embodiment 182 is the gRNA of embodiment 181, wherein the modification is 2′-fluoro.
Embodiment 183 is the gRNA of embodiment 181, wherein the modification is 2′-OMe or 2′-H.
Embodiment 184 is the gRNA of embodiment 181, wherein the modification is PS.
Embodiment 185 is the gRNA of any one of embodiments 161-184, wherein at least 1, 2, 3, 4, or 5 of nucleotides 6-10 from the 5′ terminus comprise a YA modification, optionally wherein the modification comprises 2′-fluoro, 2′-H, 2′-OMe, inosine, or PS.
Embodiment 186 is the gRNA of embodiment 185, wherein the modification is PS.
Embodiment 187 is the gRNA of embodiment 185, wherein the modification is 2′-fluoro or 2′-H.
Embodiment 188 is the gRNA of embodiment 185, wherein the modification is 2′-OMe.
Embodiment 189 is the gRNA of any one of embodiments 161-188, comprising any one or more of the following:
   a. 1, 2, 3, 4, 5, 6, 7, or 8 YA modifications of nucleotides 8-11, 13-14, and 17-18 from the 5′ end of the 5′ terminus, wherein the YA modifications are optionally 2′-fluoro modifications, and a modification other than 2′-fluoro at one or more of nucleotides 6-10 from the 5′ end of the 5′ terminus;
   b. a YA modification other than PS at one or more of nucleotides 8-11, 13-14, and 17-18 from the 5′ end of the 5′ terminus, and 1, 2, 3, 4, or 5 YA modifications at nucleotides 6-10 from the 5′ end of the 5′ end of the 5′ terminus, optionally wherein the modifications are PS modifications;
   c. 1, 2, 3, 4, 5, 6, 7, or 8 YA modifications at nucleotides 8-11, 13-14, and 17-18 from the 5′ end of the 5′ terminus, wherein the YA modifications are optionally 2′-fluoro modifications, and modifications other than 2′-fluoro at nucleotides 6-10 from the 5′ end of the 5′ terminus;
   d. YA modifications other than PS at each of nucleotides 8-11, 13-14, and 17-18 from the 5′ end of the 5′ end of the 5′ terminus, and 1, 2, 3, 4, or 5 YA modifications at nucleotides 6-10 from the 5′ end of the 5′ terminus, wherein the modifications are optionally PS modifications;
   e. 1, 2, 3, 4, 5, 6, 7, or 8 YA modifications at nucleotides 8-11, 13-14, and 17-18 from the 5′ end of the 5′ terminus, wherein the YA modifications are optionally 2′-fluoro modifications, and one or more PS modification at any one of nucleotides 6-10 from the 5′ end of the 5′ terminus;
   f. at least one 2′-fluoro modification at any one of nucleotides 8-11, 13-14, and 17-18 from the 5′ end of the 5′ terminus, and 1, 2, 3, 4, or 5 YA modifications of nucleotides 6-10 from the 5′ end of the 5′ terminus, wherein the modifications are optionally PS modifications;
   g. 1, 2, 3, 4, 5, 6, 7, or 8 YA modifications of nucleotides 8-11, 13-14, and 17-18 from the 5′ end of the 5′ terminus, wherein the YA modifications are optionally 2′-fluoro modifications, and a PS modification at each of nucleotides 6-10 from the 5′ end of the 5′ terminus; or
   h. a 2′-fluoro modification at each of nucleotides 8-11, 13-14, and 17-18 from the 5′ end of the 5′ terminus, and 1, 2, 3, 4, or 5 YA modifications of nucleotides 6-10 from the 5′ end of the 5′ terminus, wherein the modifications are optionally PS modifications.
Embodiment 190 is the gRNA of any one of embodiments 161-189, wherein:
   a. nucleotides 4-20 from the 5′ end of the 5′ terminus comprise at least 2, 3, or 4 modified YA sites including a first modified YA site comprising a 2′-OMe modification and a second modified YA site comprising a 2′-fluoro modification or a PS modification;
   b. nucleotides 4-20 from the 5′ end of the 5′ terminus comprise at least 2, 3, or 4 modified YA sites including a first modified YA site comprising a 2′-fluoro modification and a second modified YA site comprising a 2′-OMe modification or a PS modification;
   c. nucleotides 4-20 from the 5′ end of the 5′ terminus comprise at least 2, 3, or 4 modified YA sites including a first modified YA site comprising a PS modification and a second modified YA site comprising a 2′-OMe modification or a 2′-fluoro modification;
   d. nucleotides 4-20 from the 5′ end of the 5′ terminus comprise at least 2, 3, or 4 modified YA sites including a YA modification;
   e. nucleotides 4-20 from the 5′ end of the 5′ terminus comprise at least 3 or 4 modified YA sites including a first modified YA site comprising a 2′-OMe modification, a second modified YA site comprising a 2′-fluoro modification, and a third modified YA site comprising a PS modification;
   f. nucleotides 4-20 from the 5′ end of the 5′ terminus comprise at least 3 or 4 modified YA sites including a first modified YA site comprising a 2′-OMe modification, a second modified YA site comprising a 2′-fluoro modification, a third modified YA site comprising a 2′-fluoro modification, and a fourth modified YA site comprising a PS modification;
   g. nucleotides 4-20 from the 5′ end of the 5′ terminus comprise at least 3 or 4 modified YA sites including a YA modification;
   h. nucleotides 4-20 from the 5′ end of the 5′ terminus comprise at least 4 modified YA sites including a first modified YA site comprising a 2′-OMe modification, a second modified YA site comprising a 2′-fluoro modification, a third modified YA site comprising a PS modification, and a fourth modified YA site comprising a PS modification; or
   i. nucleotides 4-40 from the 5′ end of the 5′ terminus comprise at least 4 modified YA sites including a YA modification.
Embodiment 191 is the gRNA of any one of embodiments 161-190, wherein nucleotides 4-20 from the 5′ end of the 5′ terminus comprise at least 5 modified YA sites.
Embodiment 192 is the gRNA of any one of embodiments 161-191, wherein the at least 5 modified YA sites include a fifth modified YA site comprising a PS modification, optionally wherein the third modified YA site comprises a 2′-fluoro modification.
Embodiment 193 is the gRNA of any one of embodiments 161-192, wherein the first, second, and (if applicable) third, fourth, and fifth of the at least 5 modified YA sites are arranged in the 5′ to 3′ direction.
Embodiment 194 is the gRNA of any one of embodiments 161-193, wherein the first, second, and (if applicable) third, fourth, and fifth of the at least 5 modified YA sites are not arranged in the 5′ to 3′ direction.
Embodiment 195 is the gRNA of any one of embodiments 161-194, wherein nucleotides 4-20 from the 5′ end of the 5′ terminus comprise at least 2, 3, 4, or 5 modified YA sites comprising a deoxyribonucleotide, optionally wherein the deoxyribonucleotide is the pyrimidine of the YA sites.
Embodiment 196 is the gRNA of any one of embodiments 161-195, wherein:
   a. at least 1, 2, 3, or 4 of nucleotides 8-11 from the 5′ end of the 5′ terminus comprise a YA modification, which is optionally a 2′-fluoro modification;
   b. at least 1, 2, 3, 4, 5, 6, 7, or 8 of nucleotides 8-11, 13, 14, 17, and 18 from the 5′ end of the 5′ terminus comprise a YA modification, optionally wherein the YA modifications are 2′-OMe if present at nucleotides 8-11 and 2′-fluoro if present at nucleotides 13, 14, 17, or 18;
   c. at least one or both of nucleotides 17 and 18 from the 5′ end of the 5′ terminus comprise a YA modification, which is optionally a 2′-fluoro modification;
   d. at least one or both of nucleotides 17 and 18 from the 5′ end of the 5′ terminus comprise a YA modification, which is optionally a 2′-fluoro modification; or
   e. at least 1, 2, 3, 4, 5, 6, 7, 8, 9, 10, 11, 12, or 13 of nucleotides 4-14, 17, and 18 from the 5′ end of the 5′ terminus comprise a YA modification, which is optionally a 2′-fluoro modification.
Embodiment 197 is the gRNA of any one of embodiments 161-196, wherein at least 1, 2, 3, 4, 5, or 6 of nucleotides 4-10 from the 5′ end of the 5′ terminus comprise a YA modification, which is optionally a 2′-OMe modification.
Embodiment 198 is the gRNA of any one of embodiments 161-197, wherein nucleotides 4-10 from the 5′ end of the 5′ terminus comprise a YA modification, which is optionally a 2′-OMe modification.
Embodiment 199 is the gRNA of any one of embodiments 161-198, wherein:
   a. at least one of nucleotides 1-3 from the 5′ end of the 5′ terminus comprise a 5′ protective end modification, which is optionally a 2′-OMe modification;
   b. at least two of nucleotides 1-3 from the 5′ end of the 5′ terminus comprise a 5′ protective end modification, which is optionally a 2′-OMe modification; or
   c. each of nucleotides 1-3 from the 5′ end of the 5′ terminus comprise a 5′ protective end modification, which is optionally a 2′-OMe modification.
Embodiment 200 is the gRNA of any one of embodiments 161-199, wherein at least 1, 2, 3, 4, or 5 of nucleotides 11, 13, 14, 17, and 18 from the 5′ end of the 5′ terminus comprise a 5′ end modification, which is optionally a 2′-fluoro modification.
Embodiment 201 is the gRNA of any one of embodiments 161-200, wherein nucleotide 15 from the 5′ end of the 5′ terminus is unmodified or modified only with phosphorothioate.
Embodiment 202 is the gRNA of any one of embodiments 161-200, wherein nucleotide 16 from the 5′ end of the 5′ terminus is unmodified or modified only with phosphorothioate.
Embodiment 203 is the gRNA of any one of the preceding embodiments, wherein nucleotide 3 from the 5′ end of the 5′ terminus is unmodified or modified only with phosphorothioate.
Embodiment 204 is the gRNA of any one of embodiments 161-203, which is a crRNA or dgRNA.
Embodiment 205 is the gRNA of any one of embodiments 161-203, which is an sgRNA.
Embodiment 206 is the gRNA of any one of embodiments 161-203, which is a short-sgRNA.
Embodiment 207 is the gRNA of any one of embodiment 205 or 206, comprising a YA modification of conserved region YA site 1.
Embodiment 208 is the gRNA of any one of embodiments 205-207, comprising a YA modification of conserved region YA site 2.
Embodiment 209 is the gRNA of any one of embodiments 205-208, comprising a YA modification of conserved region YA site 3.
Embodiment 210 is the gRNA of any one of embodiments 205-209, comprising a YA modification of conserved region YA site 4.
Embodiment 211 is the gRNA of any one of embodiments 205-210, comprising a YA modification of conserved region YA site 5.
Embodiment 212 is the gRNA of any one of embodiments 205-211, comprising a YA modification of conserved region YA site 6.
Embodiment 213 is the gRNA of any one of embodiments 205-212, comprising a YA modification of conserved region YA site 7.
Embodiment 214 is the gRNA of any one of embodiments 205-213, comprising a YA modification of conserved region YA site 8.
Embodiment 215 is the gRNA of any one of embodiments 205-214, comprising a YA modification of conserved region YA site 9.
Embodiment 216 is the gRNA of any one of embodiments 205-215, comprising a YA modification of conserved region YA site 10.
Embodiment 217 is the gRNA of any one of embodiments 205-216, comprising:
   a. YA modifications of conserved region YA sites 2, 3, 4, and 10;
   b. YA modifications of conserved region YA sites 2, 3, and 4;
   c. YA modifications of conserved region YA sites 2, 3, and 10;
   d. YA modifications of conserved region YA sites 2, 4, and 10;
   e. YA modifications of conserved region YA sites 3, 4, and 10;
   f. YA modifications of conserved region YA sites 2 and 10;
   g. YA modifications of conserved region YA sites 2 and 4;
   h. YA modifications of conserved region YA sites 2 and 3;
   i. YA modifications of conserved region YA sites 3 and 4;
   j. YA modifications of conserved region YA sites 3 and 10; or
   k. YA modifications of conserved region YA sites 4 and 10.
Embodiment 218 is the gRNA of any one of embodiments 205-217, comprising:
   a. YA modifications of conserved region YA sites 1 and 5;
   b. YA modifications of conserved region YA sites 1 and 6;
   c. YA modifications of conserved region YA sites 1 and 7;
   d. YA modifications of conserved region YA sites 1 and 8;
   e. YA modifications of conserved region YA sites 1 and 9;
   f. YA modifications of conserved region YA sites 8 and 5;
   g. YA modifications of conserved region YA sites 8 and 6;
   h. YA modifications of conserved region YA sites 8 and 7; or
   i. YA modifications of conserved region YA sites 8 and 9;
      optionally wherein the sgRNA further comprises YA modifications of conserved region YA sites 2, 3, 4, and 10.
Embodiment 219 is the gRNA of any one of embodiments 205-218, wherein at least one modified YA site comprises a 2′-OMe modification, optionally at the pyrimidine of the YA site.
Embodiment 220 is the gRNA of any one of embodiments 205-219, wherein at least one modified YA site comprises a 2′-fluoro modification, optionally at the pyrimidine of the YA site.
Embodiment 221 is the gRNA of any one of embodiments 205-220, wherein at least one modified YA site comprises a PS modification, optionally at the pyrimidine of the YA site.
Embodiment 222 is the gRNA of any one of embodiments 205-221, wherein at least 2, 3, 4, 5, 6, 7, 8, 9, or 10 modified YA sites comprise a 2′-OMe modification, optionally at the pyrimidines of the YA sites.
Embodiment 223 is the gRNA of any one of embodiments205-222, wherein at least 2, 3, 4, 5, 6, 7, 8, 9, or 10 modified YA sites comprise a 2′-fluoro modification, optionally at the pyrimidines of the YA sites.
Embodiment 224 is the gRNA of any one of embodiments205-223, wherein at least 2, 3, 4, 5, 6, 7, 8, 9, or 10 modified YA sites comprise a PS modification, optionally at the pyrimidines of the YA sites.
Embodiment 225 is the gRNA of any one of embodiments205-224, wherein at least 2, 3, 4, 5, 6, 7, 8, 9, or 10 modified YA sites comprise a ribose modification at the 2′ position, optionally at the pyrimidines of the YA sites, and optionally chosen from a 2′-O-alkyl, 2′-H, and 2′-fluoro modification.
Embodiment 226 is the gRNA of any one of embodiments 205-225, wherein:
   a. conserved region YA sites 1 and 8 comprise 2′-fluoro modifications, optionally at the pyrimidines of the YA sites;
   b. conserved region YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites;
   c. conserved region YA site 1 comprises a 2′-fluoro modification and conserved region YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites;
   d. conserved region YA site 8 comprises a 2′-fluoro modification and conserved region YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites;
   e. conserved region YA site 1 comprises a 2′-fluoro modification at the pyrimidine of the YA sites and YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites;
   f. conserved region YA site 8 comprises a 2′-fluoro modification at the pyrimidine of the YA site and YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites;
   g. conserved region YA sites 1 and 8 comprise 2′-fluoro modifications and conserved region YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites; or
   h. conserved region YA sites 1 and 8 comprise 2′-fluoro modifications at the pyrimidines of the YA sites and conserved region YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites.
Embodiment 227 is the gRNA of any one of embodiments 205-226, wherein conserved region YA sites 7 and 9 comprise YA modifications, which are optionally 2′-OMe modifications.
Embodiment 228 is the gRNA of any one of embodiments 205-227, wherein conserved region YA sites 5, 6, 7, and 9 comprise YA modifications, which are optionally 2′-OMe modifications.
Embodiment 229 is the gRNA of any one of embodiments 205-228, wherein conserved region YA site 8 comprises a 2′-fluoro modification.
Embodiment 230 is the gRNA of any one of embodiments 205-229, wherein conserved region YA site 8 comprises a deoxyribonucleotide modification.
Embodiment 231 is the gRNA of any one of embodiments 205-230, wherein conserved region YA site 8 is abolished by a base substitution, optionally wherein the base substitution eliminates the uracil of YA site 8, further optionally wherein the base substitution is a uracil to guanine substitution.
Embodiment 232 is the gRNA of any one of embodiments 205-231, wherein conserved region YA site 1 comprises a 2′-fluoro modification.
Embodiment 233 is the gRNA of any one of embodiments 205-232, wherein conserved region YA site 1 comprises a PS modification.
Embodiment 234 is the gRNA of any one of embodiments 205-233, wherein 1, 2, 3, 4, 5, 6, or 7 of LS5, LS7, LS8, LS9, LS10, LS11, and LS12 comprise modifications, optionally wherein the modifications are 2′-fluoro and/or 2′-OMe modifications.
Embodiment 235 is the gRNA of any one of embodiments 205-234, wherein modifications at LS5, LS7, LS9, and LS11, if present, comprise 2′-fluoro modifications, optionally wherein each of LS5, LS7, LS9, and LS11 comprise 2′-fluoro modifications.
Embodiment 236 is the gRNA of any one of embodiments 205-235, wherein modifications at LS8, LS10, and LS12, if present, comprise 2′-OMe modifications, optionally wherein each of LS8, LS10, and LS12 comprise 2′-OMe modifications.
Embodiment 237 is the gRNA of any one of embodiments 205-236, wherein 1, 2, 3, 4, 5, 6, 7, 8, 9, or 10 of N2, N3, N4, N5, N6, N7, N10, N11, N16, and N17 comprise modifications, which are optionally 2′-OMe modifications.
Embodiment 238 is the gRNA of any one of embodiments 205-237, wherein H2-2 comprises a modification, optionally wherein H2 is otherwise unmodified.
Embodiment 239 is the gRNA of any one of embodiments 205-238, wherein H2-2 comprises a 2′-OMe modification.
Embodiment 240 is the gRNA of any one of embodiments 205-239, wherein US3, US9, and US12 comprise modifications, optionally wherein the US is otherwise unmodified.
Embodiment 241 is the gRNA of any one of embodiments 205-240, wherein US3, US9, and US12 comprise 2′-OMe modifications.
Embodiment 242 is the gRNA of any one of embodiments 205-241, wherein nucleotides 6-10 from the 5′ end of the 5′ terminus comprise a PS modification and nucleotides 8-11, 13, 14, 17, and 18 from the 5′ end of the 5′ terminus comprise a 2′-fluoro modification.
Embodiment 243 is the gRNA of any one of embodiments 205-242, wherein each guide region YA site comprises a 2′-fluoro modification, optionally excepting nucleotides 15 and/or 16 from the 5′ end of the 5′ terminus.
Embodiment 244 is the gRNA of any one of embodiments 205-243, wherein nucleotides 4, 8, and 11 from the 5′ end of the 5′ terminus comprise YA modifications, optionally wherein nucleotide 4 comprises a 2′-OMe modification and nucleotides 8 and 11 comprise a 2′-fluoro modification.
Embodiment 245 is the gRNA of any one of embodiments 205-244, wherein 1, 2, 3, 4, 5, 6, 7, 8, 9, 10, 11, 12, 13, 14, 15, or more modified YA sites comprise a YA modification at the pyrimidine position of the YA site.
Embodiment 246 is the gRNA of embodiment 245, wherein 1, 2, 3, 4, 5, 6, 7, 8, 9, or 10 modified conserved region YA sites comprise a YA modification at the pyrimidine position of the YA site.
Embodiment 247 is the gRNA of any one of embodiments 205-246, wherein 1, 2, 3, 4, 5, 6, 7, 8, 9, 10, 11, 12, 13, 14, 15, or more modified YA sites comprise a YA modification at the adenine position of the YA site.
Embodiment 248 is the gRNA of embodiment 247, wherein 1, 2, 3, 4, 5, 6, 7, 8, 9, or 10 modified conserved region YA sites comprise a YA site modification at the adenine position of the YA site.
Embodiment 249 is the gRNA of any one of embodiments 205-248, comprising:
   a. a modification of H1-1;
   b. a modification of H2-1; or
   c. modifications of H1-1 and H2-1.
Embodiment 250 is the gRNA of embodiment 249, wherein H1-1 and/or H2-1 comprises a 2′-OMe modification.
Embodiment 251 is the gRNA of embodiment 250, wherein H1-1 and/or H2-1 comprises a 2′-fluoro modification.
Embodiment 252 is the gRNA of embodiment 251, wherein H1-1 and/or H2-1 comprises a PS modification.
Embodiment 253 is the gRNA of any one of embodiments 205-252, comprising a modification at B3, optionally wherein B6 does not comprise a 2′-OMe modification or comprises a modification other than 2′-OMe.
Embodiment 254 is the gRNA of any one of embodiments 205-253, comprising a modification at B4, optionally wherein B6 does not comprise a 2′-OMe modification or comprises a modification other than 2′-OMe.
Embodiment 255 is the gRNA of any one of embodiments 205-254, comprising a modification at B5, optionally wherein B6 does not comprise a 2′-OMe modification or comprises a modification other than 2′-OMe.
Embodiment 256 is the gRNA of any one of embodiments 205-255, comprising a modification at LS10, optionally wherein LS10 comprises a modification other than 2′-fluoro.
Embodiment 257 is the gRNA of any one of embodiments 205-256, comprising a modification at N2.
Embodiment 258 is the gRNA of any one of embodiments 205-257, comprising a modification at N3.
Embodiment 259 is the gRNA of any one of embodiments 205-258, comprising a modification at N4.
Embodiment 260 is the gRNA of any one of embodiments 205-259, comprising a modification at N5.
Embodiment 261 is the gRNA of any one of embodiments 205-260, comprising a modification at N6.
Embodiment 262 is the gRNA of any one of embodiments 205-261, comprising a modification at N7.
Embodiment 263 is the gRNA of any one of embodiments 205-262, comprising a modification at N10.
Embodiment 264 is the gRNA of any one of embodiments 205-263, comprising a modification at N11.
Embodiment 265 is the gRNA of any one of embodiments 205-264, wherein:
   a. nucleotide 8 from the 5′ end of the 5′ terminus does not comprise a 2′-fluoro modification;
   b. nucleotide 9 from the 5′ end of the 5′ terminus does not comprise a 2′-fluoro modification;
   c. nucleotide 10 from the 5′ end of the 5′ terminus does not comprise a 2′-fluoro modification;
   d. nucleotide 11 from the 5′ end of the 5′ terminus does not comprise a 2′-fluoro modification;
   e. nucleotide 13 from the 5′ end of the 5′ terminus does not comprise a 2′-fluoro modification;
   f. nucleotide 14 from the 5′ end of the 5′ terminus does not comprise a 2′-fluoro modification;
   g. nucleotide 17 from the 5′ end of the 5′ terminus does not comprise a 2′-fluoro modification; and/or
   h. nucleotide 18 from the 5′ end of the 5′ terminus does not comprise a 2′-fluoro modification.
Embodiment 266 is the gRNA of any one of embodiments 205-265, wherein:
   a. nucleotide 6 from the 5′ end of the 5′ terminus does not comprise a 2′-fluoro modification;
   b. nucleotide 7 from the 5′ end of the 5′ terminus does not comprise a 2′-fluoro modification;
   c. nucleotide 8 from the 5′ end of the 5′ terminus does not comprise a 2′-fluoro modification;
   d. nucleotide 9 from the 5′ end of the 5′ terminus does not comprise a 2′-fluoro modification; and/or
   e. nucleotide 10 from the 5′ end of the 5′ terminus does not comprise a 2′-fluoro modification.
Embodiment 267 is the gRNA of any one of embodiments 205-266, wherein:
   a. nucleotide 6 from the 5′ end of the 5′ terminus does not comprise a phosphorothioate linkage;
   b. nucleotide 7 from the 5′ end of the 5′ terminus does not comprise a phosphorothioate linkage;
   c. nucleotide 8 from the 5′ end of the 5′ terminus does not comprise a phosphorothioate linkage;
   d. nucleotide 9 from the 5′ end of the 5′ terminus does not comprise a phosphorothioate linkage; and/or
   e. nucleotide 10 from the 5′ end of the 5′ terminus does not comprise a phosphorothioate linkage.
Embodiment 268 is the gRNA of any one of embodiments 205-267, wherein:
   a. nucleotide 7 from the 5′ end of the 5′ terminus does not comprise a 2′-OMe modification;
   b. nucleotide 8 from the 5′ end of the 5′ terminus does not comprise a 2′-OMe modification;
   c. nucleotide 9 from the 5′ end of the 5′ terminus does not comprise a 2′-OMe modification; and/or
   d. nucleotide 10 from the 5′ end of the 5′ terminus does not comprise a 2′-OMe modification.
Embodiment 269 is the gRNA of any one of embodiments 205-268, wherein nucleotide 20 from the 5′ end of the 5′ terminus does not comprise a 2′-OMe modification.
Embodiment 270 is the gRNA of any one of embodiments 205-269, wherein the guide RNA comprises a 2′-fluoro modification at any one or more of nucleotides 1-11 and 13-20 from the 5′ end of the 5′ terminus and nucleotide 12 from the 5′ end of the 5′ terminus does not comprise a 2′-fluoro modification.
Embodiment 271 is the gRNA of any one of embodiments 205-270, wherein the guide RNA comprises a 2′-fluoro modification at any one or more of nucleotides 1-20 from the 5′ end of the 5′ terminus and:
   a. nucleotide 11 from the 5′ end of the 5′ terminus does not comprise a 2′-fluoro modification;
   b. nucleotide 12 from the 5′ end of the 5′ terminus does not comprise a 2′-fluoro modification;
   c. nucleotide 13 from the 5′ end of the 5′ terminus does not comprise a 2′-fluoro modification;
   d. nucleotide 14 from the 5′ end of the 5′ terminus does not comprise a 2′-fluoro modification;
   e. nucleotide 17 from the 5′ end of the 5′ terminus does not comprise a 2′-fluoro modification; and/or
   f. nucleotide 18 from the 5′ end of the 5′ terminus does not comprise a 2′-fluoro modification.
Embodiment 272 is the gRNA of any one of embodiments 205-271, wherein:
   a. B2 does not comprise a 2′-OMe modification;
   b. B3 does not comprise a 2′-OMe modification;
   c. B4 does not comprise a 2′-OMe modification; and/or
   d. B5 does not comprise a 2′-OMe modification.
Embodiment 273 is the gRNA of any one of embodiments 205-272, wherein:
   a. LS1 does not comprise a 2′-OMe modification;
   b. LS8 does not comprise a 2′-OMe modification; and/or
   c. LS10 does not comprise a 2′-OMe modification.
Embodiment 274 is the gRNA of any one of embodiments 205-273, wherein:
   a. N2 does not comprise a 2′-OMe modification;
   b. N3 does not comprise a 2′-OMe modification;
   c. N4 does not comprise a 2′-OMe modification;
   d. N5 does not comprise a 2′-OMe modification;
   e. N6 does not comprise a 2′-OMe modification;
   f. N7 does not comprise a 2′-OMe modification;
   g. N10 does not comprise a 2′-OMe modification;
   h. N11 does not comprise a 2′-OMe modification;
   i. N16 does not comprise a 2′-OMe modification; and/or
   j. N17 does not comprise a 2′-OMe modification.
Embodiment 275 is the gRNA of any one of embodiments 205-274, wherein:
   a. H1-2 does not comprise a phosphorothioate linkage;
   b. H1-3 does not comprise a phosphorothioate linkage;
   c. H1-4 does not comprise a phosphorothioate linkage;
   d. H1-5 does not comprise a phosphorothioate linkage;
   e. H1-6 does not comprise a phosphorothioate linkage;
   f. H1-7 does not comprise a phosphorothioate linkage;
   g. H1-8 does not comprise a phosphorothioate linkage;
   h. H1-9 does not comprise a phosphorothioate linkage;
   i. H1-10 does not comprise a phosphorothioate linkage;
   j. H2-1 does not comprise a phosphorothioate linkage;
   k. H2-2 does not comprise a phosphorothioate linkage;
   l. H2-3 does not comprise a phosphorothioate linkage;
   m. H2-4 does not comprise a phosphorothioate linkage;
   n. H2-5 does not comprise a phosphorothioate linkage;
   o. H2-6 does not comprise a phosphorothioate linkage;
   p. H2-7 does not comprise a phosphorothioate linkage;
   q. H2-8 does not comprise a phosphorothioate linkage;
   r. H2-9 does not comprise a phosphorothioate linkage;
   s. H2-10 does not comprise a phosphorothioate linkage;
   t. H2-11 does not comprise a phosphorothioate linkage;
   u. H2-12 does not comprise a phosphorothioate linkage;
   v. H2-13 does not comprise a phosphorothioate linkage;
   w. H2-14 does not comprise a phosphorothioate linkage; and/or
   x. H2-15 does not comprise a phosphorothioate linkage.
Embodiment 276 is a gRNA which is an sgRNA comprising modifications at:
   a. nucleotides 6-10 from the 5′ end of the 5′ terminus, which are optionally PS modifications;
   b. nucleotides 8-11, 13, 14, 17, and 18 from the 5′ end of the 5′ terminus, which are optionally 2′-fluoro modifications; and
   c. H1-1 and H2-1, which are optionally 2′-OMe modifications, or conserved region YA site 1 or 8.
Embodiment 277 is a gRNA which is an sgRNA comprising YA modifications at:
   a. conserved region YA sites 1, 5, 6, 7, and 9, which are optionally 2′-OMe modifications; and
   b. conserved region YA site 8, which is optionally a 2′-fluoro modification.
Embodiment 278 is a gRNA comprising YA modifications at four guide region YA sites, wherein at least one of the YA sites is at or after nucleotide 8 from the 5′ end of the 5′ terminus, and wherein:
   a. the first YA site comprises a 2′-OMe modification;
   b. the second YA site comprises a 2′-fluoro modification;
   c. the third YA site comprises a 2′-fluoro or PS modification; and
   d. the fourth YA site comprises a PS modification,
      optionally wherein the first, second, third, and fourth YA sites are arranged in the 5′ to 3′ direction.
Embodiment 279 is the gRNA of embodiment 278, wherein the third YA site comprises a PS modification.
Embodiment 280 is the gRNA of any one of embodiments 278-279, wherein the third YA site comprises a 2′-fluoro modification.
Embodiment 281 is the gRNA of any one of embodiments 278-280, further comprising a fifth YA site comprising a PS modification, which is optionally 3′ of the fourth YA site.
Embodiment 282 is the gRNA of any one of the embodiments 205-281, wherein conserved region YA sites 1, 5, 6, 7, and 9 comprise YA modifications, which are optionally 2′-OMe modifications; and conserved region YA site 8 comprises a modification, which is optionally a 2′-fluoro modification.
Embodiment 283 is a gRNA which is an sgRNA comprising YA modifications at:
   a. nucleotide 4 from the 5′ end of the 5′ terminus, wherein the YA modification is optionally a 2′-OMe modification;
   b. nucleotides 6-10 from the 5′ end of the 5′ terminus, which are optionally PS modifications;
   c. nucleotides 8-11, 13, 14, 17, and 18 from the 5′ end of the 5′ terminus, which are optionally 2′-fluoro modifications;
   d. LS5, LS7, LS9, and LS11, which are optionally 2′-fluoro modifications;
   e. LS8, LS10, and LS12, which are optionally 2′-OMe modifications;
   f. N2, N3, N4, N5, N6, N7, N10, N11, N16, and N17, which are optionally 2′-OMe modifications; and
   g. N14, which is optionally a 2′-fluoro modification.
Embodiment 284 is the gRNA of any one of embodiment 161-, wherein one or more of the following are true:
   a. nucleotide 4 from the 5′ end of the 5′ terminus comprises a 2′-OMe modification;
   b. nucleotides 6-10 from the 5′ end of the 5′ terminus comprise PS modifications;
   c. nucleotides 8-11, 13, 14, 17, and 18 from the 5′ end of the 5′ terminus comprise 2′-fluoro modifications;
   d. LS5, LS7, LS9, and LS11 comprise 2′-fluoro modifications;
   e. LS8, LS10, and LS12 comprise 2′-OMe modifications;
   f. N2, N3, N4, N5, N6, N7, N10, N11, N16, and N17 comprise 2′-OMe modifications; and
   g. N14 comprises a 2′-fluoro modification.
Embodiment 285 is the gRNA of any one of embodiments 161-284, wherein at least one YA modification comprises a modification of the pyrimidine position of the YA site.
Embodiment 286 is the gRNA of any one of embodiments 161-285, wherein at least one YA modification comprises a modification of the adenine position of the YA site.
Embodiment 287 is the gRNA of any one of embodiments 161-286, wherein at least 2, 3, 4, 5, 6, 7, 8, 9, or 10 YA sites comprise YA modifications at the pyrimidines positions of the YA sites.
Embodiment 288 is the gRNA of any one of embodiments 161-287, wherein at least 2, 3, 4, 5, 6, 7, 8, 9, or 10 YA sites comprise YA modifications at the adenine positions of the YA sites.
Embodiment 289 is the gRNA of any one of embodiments 161-288, wherein at least one YA modification comprises a 2′-OMe modification.
Embodiment 290 is the gRNA of any one of embodiments 161-289, wherein at least 2, 3, 4, 5, 6, 7, 8, 9, or 10 YA sites comprise a 2′-OMe modification.
Embodiment 291 is the gRNA of any one of embodiments 161-290, wherein each modified conserved region YA site comprises a modification at the pyrimidine position of the YA site.
Embodiment 292 is the gRNA of any one of embodiments 161-291, wherein each modified guide region YA site, or each modified conserved region and guide region YA site, comprises a modification at the pyrimidine position of the YA site.
Embodiment 293 is the gRNA of any one of embodiments 161-292, wherein each modified conserved region YA site comprises a modification at the adenine position of the YA site.
Embodiment 294 is the gRNA of any one of embodiments 161-293, wherein each modified guide region YA site, or each modified conserved region and guide region YA site, comprises a modification at the adenine position of the YA site.
Embodiment 295 is the gRNA of any one of embodiments 161-294, which is an sgRNA comprising a modification at LS5.
Embodiment 296 is the gRNA of any one of embodiments 161-295, which is an sgRNA comprising a modification at LS7.
Embodiment 297 is the gRNA of any one of embodiments 161-296, which is an sgRNA comprising a modification at LS9, optionally wherein if LS9 is modified and LS5, LS7, and LS12 are not, then the modification of LS9 is other than 2′-fluoro.
Embodiment 298 is the gRNA of any one of embodiments 161-297, which is an sgRNA comprising a modification at LS12, optionally wherein if LS12 is modified and LS9 is not, then the modification of LS12 is other than 2′-OMe.
Embodiment 299 is the gRNA of any one of embodiments 161-298, which is an sgRNA comprising at least one YA modification that stabilizes a secondary structure, optionally wherein the secondary structure is the lower stem.
Embodiment 300 is the gRNA of any one of embodiments 161-299, which is an sgRNA comprising at least one modification of LS8 and/or LS11, optionally wherein the modification of LS8 and/or LS11 stabilizes a secondary structure.
Embodiment 301 is the gRNA of any one of embodiments 161-300, comprising a YA modification that stabilizes a secondary structure chosen from:
   a. ENA;
   b. LNA; or
   c. a bicyclic ribose modification.
Embodiment 302 is the gRNA of any one of embodiments 161-301, which is an sgRNA comprising a modification at N6.
Embodiment 303 is the gRNA of any one of embodiments 161-302, which is an sgRNA comprising a modification at N14.
Embodiment 304 is the gRNA of any one of embodiments 161-303, which is an sgRNA comprising a modification at N17, optionally wherein if N17 is modified and N6 and N14 are not, then the modification of N17 is other than 2′-fluoro and other than 2′-OMe.
Embodiment 305 is the gRNA of any one of embodiments 161-304, wherein at least 1, 2, or 3 of nucleotides 1-3 from the 5′ end of the 5′ terminus comprise deoxyribonucleotides, optionally wherein nucleotides 1-3 from the 5′ end of the 5′ terminus comprise PS modifications.
Embodiment 306 is the gRNA of any one of embodiments 161-305, wherein the gRNA is an sgRNA and at least 1, 2, or 3 of nucleotides 1-3 from the 3′ end of the 3′ terminus comprise deoxyribonucleotides, optionally wherein nucleotides 2-3 from the 3′ end of the 3′ terminus comprise PS modifications.
Embodiment 307 is the gRNA of any one of embodiments 161-306, wherein the gRNA is an sgRNA and nucleotide 4 from the 3′ end of the 3′ terminus comprises a PS modification, optionally wherein nucleotide 4 from the 3′ end of the 3′ terminus comprises a 2′-OMe modification.
Embodiment 308 is the gRNA of any one of embodiments 161-307, wherein the gRNA is an sgRNA and hairpin 2 comprises deoxyribonucleotides, optionally wherein all or all but 1, 2, 3, 4, 5, 6, 7, 8, 9, or 10 nucleotides of hairpin 1 and hairpin 2 are deoxyribonucleotides.
Embodiment 309 is the gRNA of any one of embodiments 161-308, wherein the gRNA is an sgRNA and hairpin 1 and hairpin 2 comprise deoxyribonucleotides, optionally wherein all or all but 1, 2, 3, 4, 5, 6, 7, 8, 9, 10, 11, 12, or 13 nucleotides of hairpin 1 and hairpin 2 are deoxyribonucleotides.
Embodiment 310 is the gRNA of any one of embodiments 161-309, wherein the gRNA is an sgRNA and all or all but 1, 2, 3, 4, 5, 6, 7, 8, 9, 10, 11, 12, or 13 nucleotides from the beginning of hairpin 1 to the 3′ end of the sgRNA are deoxyribonucleotides, optionally wherein nucleotides 1-3 from the 3′ end of the 3′ terminus are deoxyribonucleotides.
Embodiment 311 is the gRNA of any one of embodiments 161-310, wherein the gRNA is an sgRNA and the upper stem comprises deoxyribonucleotides.
Embodiment 312 is the gRNA of any one of embodiments 161-311, wherein the gRNA is an sgRNA and all or all but 1, 2, 3, 4, 5, 6, 7, 8, 9, or 10 nucleotides of the upper stem are deoxyribonucleotides.
Embodiment 313 is the gRNA of any one of embodiments 161-312, wherein at least 1, 2, or 3 of nucleotides 1-3 from the 5′ end of the 5′ terminus comprise ENA, optionally wherein nucleotides 1-3 from the 5′ end of the 5′ terminus comprise PS modifications.
Embodiment 314 is the gRNA of any one of embodiments 161-313, wherein the gRNA is an sgRNA and at least 1, 2, or 3 of nucleotides 2-4 from the 3′ end of the 3′ terminus comprise ENA, optionally wherein nucleotides 2-3 from the 3′ end of the 3′ terminus comprise PS modifications.
Embodiment 315 is the gRNA of any one of embodiments 161-314, wherein at least 1, 2, or 3 of nucleotides 1-3 from the 5′ end of the 5′ terminus comprise UNA, optionally wherein nucleotides 1-3 from the 5′ end of the 5′ terminus comprise PS modifications.
Embodiment 316 is the gRNA of any one of embodiments 161-315, wherein the gRNA is an sgRNA and at least 1, 2, or 3 of nucleotides 2-4 from the 3′ end of the 3′ terminus comprise UNA, optionally wherein nucleotides 2-3 from the 3′ end of the 3′ terminus comprise PS modifications.
Embodiment 317 is the gRNA of any one of embodiments 161-316, wherein the gRNA is an sgRNA and nucleotide 4 from the 3′ end of the 3′ terminus comprises a PS modification, optionally wherein nucleotide 4 from the 3′ end of the 3′ terminus comprises a 2′-OMe modification.
Embodiment 318 is the gRNA of any one embodiments 161-317, wherein the gRNA is an sgRNA that comprises a 3′ end modification.
Embodiment 319 is the gRNA of any one of embodiments 161-318, which is an sgRNA comprising a 3′ end modification, wherein the 3′ end modification is a protective 3′ end modification.
Embodiment 320 is the gRNA of any one embodiments 161-319, wherein the gRNA is an sgRNA that comprises a 3′ tail.
Embodiment 321 is the gRNA of embodiment 320, wherein the 3′ tail comprises 1, 2, 3, 4, 5, 6, 7, 8, 9, or 10 nucleotides.
Embodiment 322 is the gRNA of embodiment 320, wherein the 3′ tail comprises about 1-2, 1-3, 1-4, 1-5, 1-7, 1-10, at least 1-5, at least 1-3, at least 1-4, at least 1-5, at least 1-5, at least 1-7, or at least 1-10 nucleotides.
Embodiment 323 is the gRNA of any one of embodiments 161-322, which is an sgRNA comprising a modification in the hairpin region.
Embodiment 324 is the gRNA of any one of embodiments 161-323, which is an sgRNA comprising a 3′ end modification, and a modification in the hairpin region.
Embodiment 325 is the gRNA of embodiment 323 or 324, wherein the modification in the hairpin region comprises a modified nucleotide selected from 2′-O-methyl (2′-O-Me) modified nucleotide, a 2′-fluoro (2′-F) modified nucleotide, or combinations thereof.
Embodiment 326 is the gRNA of any one of embodiments 323-325, wherein the modification in the hairpin region comprises or further comprises a 2′-O-methyl (2′-O-Me) modified nucleotide.
Embodiment 327 is the gRNA of any one of embodiments 323-326, wherein the modification in the hairpin region comprises or further comprises a 2′-fluoro (2′-F) modified nucleotide.
Embodiment 328 is the gRNA of any one of embodiments 161-327, comprising 3′ and/or 5′ protective end modification(s).
Embodiment 329 is the gRNA of embodiment 328, wherein the 3′ and/or 5′ end modification comprises a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.
Embodiment 330 is the gRNA of embodiment 328 or 329, wherein the 3′ and/or 5′ end modification comprises or further comprises a 2′-O-methyl (2′-O-Me) modified nucleotide.
Embodiment 331 is the gRNA of embodiment 328 or 329, wherein the 3′ and/or 5′ end modification comprises or further comprises a 2′-fluoro (2′-F) modified nucleotide.
Embodiment 332 is the gRNA of embodiment 328 or 329, wherein the 3′ and/or 5′ end modification comprises or further comprises a phosphorothioate (PS) linkage between nucleotides.
Embodiment 333 is the gRNA of embodiment 328 or 329, wherein the 3′ and/or 5′ end modification comprises or further comprises an inverted abasic modified nucleotide.
Embodiment 334 is the gRNA of any one any one of embodiments 161-333, wherein the gRNA is an sgRNA and if the sgRNA comprises a 3′ end modification, the 3′ end modification comprises any one or more of the following:
   i. a modification of any one or more of the last 7, 6, 5, 4, 3, 2, or 1 nucleotides;
   ii. one modified nucleotide;
   iii. two modified nucleotides;
   iv. three modified nucleotides;
   v. four modified nucleotides;
   vi. five modified nucleotides;
   vii. six modified nucleotides; and
   viii. seven modified nucleotides.
Embodiment 335 is the gRNA of embodiment 334, wherein the 3′ end modification comprises a modification of between 1 and 7, between 1 and 5, between 1 and 4, or between 2 and 4 nucleotides.
Embodiment 336 is the gRNA of any one of embodiments 161-335, wherein the gRNA is an sgRNA comprising a 3′ end modification and the 3′ end modification comprises one or more of the following:
   i. a phosphorothioate (PS) linkage between nucleotides;
   ii. a 2′-O-Me modified nucleotide;
   iii. a 2′-O-moe modified nucleotide;
   iv. a 2′-F modified nucleotide;
   v. an inverted abasic modified nucleotide
   vi. ENA, UNA, and/or DNA; and
   vii. or a combination thereof.
Embodiment 337 is the gRNA of any one of embodiments 161-336, wherein the gRNA is an sgRNA comprising a 3′ tail, and the 3′ tail comprises any one or more of:
   i. a phosphorothioate (PS) linkage between nucleotides;
   ii. a 2′-O-Me modified nucleotide;
   iii. a 2′-O-moe modified nucleotide;
   iv. a 2′-F modified nucleotide;
   v. an inverted abasic modified nucleotide
   vi. ENA, UNA, and/or DNA; and
   vii. or a combination thereof.
Embodiment 338 is the gRNA of embodiment 336, wherein the 3′ end modification comprises:
   i. 1, 2, 3, 4, 5, 6, or 7 PS linkages between nucleotides;
   ii. about 1-3, 1-5, 1-6, or 1-7 PS linkages between nucleotides; or
   iii. PS linkages between each nucleotide.
Embodiment 339 is the gRNA of any one of embodiments embodiment 326-328, wherein the 3′ end modification further comprises at least one 2′-O-Me, 2′-O-moe, inverted abasic, or 2′-F modified nucleotide.
Embodiment 340 is the gRNA of any one of embodiments 326-329, wherein the 3′ end modification comprises at least one PS linkage, and wherein:
   i. there is one PS linkage, and the linkage is between the last and second to last nucleotide;
   ii. there are two PS linkages between the last three nucleotides;
   iii. there are PS linkages between any one or more of the last four nucleotides;
   iv. there are PS linkages between any one or more of the last five nucleotides; or
   v. there are PS linkages between any one or more of the last 2, 3, 4, 5, 6, 7, 8, 9, or 10 nucleotides.
Embodiment 341 is the gRNA of any one of embodiments 336-340, wherein the 3′ end modification comprises:
   i. a modification of one or more of the last 1-7 nucleotides, wherein the modification is a PS linkage, inverted abasic nucleotide, 2′-O-Me, 2′-O-moe, 2′-F, or combinations thereof;
   ii. a modification to the last nucleotide with 2′-O-Me, 2′-O-moe, 2′-F, or combinations thereof, and an optional one or two PS linkages to the next nucleotide and/or the first nucleotide of the 3′ tail;
   iii. a modification to the last and/or second to last nucleotide with 2′-O-Me, 2′-O-moe, 2′-F, or combinations thereof, and optionally one or more PS linkages;
   iv. a modification to the last, second to last, and/or third to last nucleotides with 2′-O-Me, 2′-O-moe, 2′-F, or combinations thereof, and optionally one or more PS linkages;
   v. a modification to the last, second to last, third to last, and/or fourth to last nucleotides with 2′-O-Me, 2′-O-moe, 2′-F, or combinations thereof, and optionally one or more PS linkages; or
   vi. a modification to the last, second to last, third to last, fourth to last, and/or fifth to last nucleotides with 2′-O-Me, 2′-O-moe, 2′-F, or combinations thereof, and optionally one or more PS linkages.
Embodiment 342 is the gRNA of any one of embodiments 161-341, wherein the gRNA is an sgRNA comprising a 3′ tail, wherein the 3′ tail comprises a modification of any one or more of the nucleotides present in the 3′ tail.
Embodiment 343 is the gRNA of embodiment 342, wherein the 3′ tail is fully modified.
Embodiment 344 is the gRNA of embodiment 342, wherein the 3′ tail comprises 1, 2, 3, 4, 5, 6, 7, 8, 9, 10, 1-2, 1-3, 1-4, 1-5, 1-6, 1-7, 1-8, 1-9, or 1-10 nucleotides, optionally where any one or more of these nucleotides are modified.
Embodiment 345 is the gRNA of any one of embodiments 336-344, wherein the 3′ end modification comprises any one or more of the following:
   i. the 3′ end modification as shown in any one of SEQ ID Nos: 401-532;
   ii. (i) a 2′O-Me modified nucleotide at the last nucleotide of the conserved region of an sgRNA or short-sgRNA, (ii) three consecutive 2′O-moe modified nucleotides immediately 5′ to the 2′O-Me modified nucleotide, and (iii) three consecutive PS linkages between the last three nucleotides;
   iii. (i) five consecutive 2′O-Me modified nucleotides from the 3′ end of the 3′ terminus, and (ii) three PS linkages between the last three nucleotides;
   iv. an inverted abasic modified nucleotide at the last nucleotide of the conserved region of an sgRNA or short-sgRNA;
   v. (i) an inverted abasic modified nucleotide at the last nucleotide of the conserved region of an sgRNA or short-sgRNA, and (ii) three consecutive 2′O-Me modified nucleotides at the last three nucleotides of the conserved region of an sgRNA or short-sgRNA;
   vi. (i) 15 consecutive 2′O-Me modified nucleotides from the 3′ end of the 3′ terminus, (ii) five consecutive 2′-F modified nucleotides immediately 5′ to the 2′O-Me modified nucleotides, and (iii) three PS linkages between the last three nucleotides;
   vii. (i) alternating 2′O-Me modified nucleotides and 2′-F modified nucleotides at the last 20 nucleotides of the conserved region of an sgRNA or short-sgRNA, and (ii) three PS linkages between the last three nucleotides;
   viii. (i) two or three consecutive 2′O-Me modified nucleotides, and (ii) three PS linkages between the last three nucleotides;
   ix. one PS linkage between the last and next to last nucleotides; and
   x. 15 or 20 consecutive 2′O-Me modified nucleotides, and (ii) three PS linkages between the last three nucleotides.
Embodiment 346 is the gRNA of any one of embodiments 161-345, comprising a 5′ end modification comprising any one or more of the following:
   i. a modification of any one or more of nucleotides 1-7 of the guide region;
   ii. one modified nucleotide;
   iii. two modified nucleotides;
   iv. three modified nucleotides;
   v. four modified nucleotides;
   vi. five modified nucleotides;
   vii. six modified nucleotides; and
   viii. seven modified nucleotides.
Embodiment 347 is the gRNA of any one of embodiments 161-346, comprising a 5′ end modification, wherein the 5′ end modification is a protective 5′ end modification.
Embodiment 348 is the gRNA of any one of embodiments 161-347, comprising a 5′ end modification, wherein the 5′ end modification comprises a modification of between 1 and 7, between 1 and 5, between 1 and 4, between 1 and 3, or between 1 and 2 nucleotides.
Embodiment 349 is the gRNA of any one of embodiments 161-348, comprising a 5′ end modification wherein the 5′ end modification comprises any one or more of the following:
   i. modifications of 1, 2, 3, 4, 5, 6, or 7 of the first 7 nucleotides;
   ii. modifications of about 1-3, 1-4, 1-5, 1-6, or 1-7 of the first 7 nucleotides; and
   iii. modifications at the first, second, third, fourth, fifth, sixth, and/or seventh nucleotide at the 5′ end, optionally wherein the modifications are consecutive.
Embodiment 350 is the gRNA of any one of embodiments 161-349, comprising a 5′ end modification, wherein the 5′ end modification comprises one or more of:
   i. a phosphorothioate (PS) linkage between nucleotides;
   ii. a 2′-O-Me modified nucleotide;
   iii. a 2′-O-moe modified nucleotide;
   iv. a 2′-F modified nucleotide;
   v. an inverted abasic modified nucleotide
   vi. ENA, UNA, and/or DNA; and
   vii. combinations thereof.
Embodiment 351 is the gRNA any one of embodiments 161-350, comprising a 5′ end modification wherein the 5′ end modification comprises:
   i. 1, 2, 3, 4, 5, 6, and/or 7 PS linkages between nucleotides; or
   ii. about 1-2, 1-3, 1-4, 1-5, 1-6, or 1-7 PS linkages between nucleotides.
Embodiment 352 is the gRNA of any one of embodiments 161-351, wherein the sgRNA comprises a 5′ end modification and the 5′ end modification comprises at least one 2′-O-Me, 2′-O-moe, inverted abasic, 2′-H, inosine, or 2′-F modified nucleotide.
Embodiment 353 is the gRNA of embodiment 352, wherein the 5′ end modification comprises at least one PS linkage, and wherein:
   i. there is one PS linkage, and the linkage is at nucleotide 1 of the guide region;
   ii. there are two PS linkages, and the linkages are at nucleotides 1 and 2 of the guide region;
   iii. there are PS linkages at any one or more of nucleotides 1, 2, and 3 of the guide region;
   iv. there are PS linkages at any one or more of nucleotides 1, 2, 3, and 4 of the guide region;
   v. there are PS linkages at any one or more of nucleotides 1, 2, 3, 4, and 5 of the guide region;
   vi. there are PS linkages at any one or more of nucleotides 1, 2, 3, 4, 5, and 6 of the guide region; or
   vii. there are PS linkages at any one or more of nucleotides 1, 2, 3, 4, 5, 6, and 7 of the guide region.
Embodiment 354 is the gRNA of any one of embodiments 352-353, wherein the 5′ end modification comprises:
   i. a modification of one or more of nucleotides 1-7 of the variable region, wherein the modification is a PS linkage, inverted abasic nucleotide, 2′-O-Me, 2′-O-moe, 2′-F, 2′-H, inosine, and/or combinations thereof;
   ii. a modification to the first nucleotide of the guide region with 2′-O-Me, 2′-O-moe, 2′-F, 2′-H, inosine, or combinations thereof, and an optional PS linkage to the next nucleotide;
   iii. a modification to the first and/or second nucleotide of the variable region with 2′-O-Me, 2′-O-moe, 2′-F, 2′-H, inosine, or combinations thereof, and optionally one or more PS linkages;
   iv. a modification to the first, second, and/or third nucleotides of the variable region with 2′-O-Me, 2′-O-moe, 2′-F, 2′-H, inosine, or combinations thereof, and optionally one or more PS linkages;
   v. a modification to the first, second, third, and/or fourth nucleotides of the variable region with 2′-O-Me, 2′-O-moe, 2′-F, 2′-H, inosine, or combinations thereof, and optionally one or more PS linkages; or
   vi. a modification to the first, second, third, fourth, and/or fifth nucleotides of the variable region with 2′-O-Me, 2′-O-moe, 2′-F, 2′-H, inosine, or combinations thereof, and optionally one or more PS linkages.
Embodiment 355 is the gRNA of any one of embodiments 161-354, comprising a 5′ end modification, wherein the 5′ end modification comprises any one or more of the following:
   i. a 5′ end modification as shown in any one of SEQ ID Nos: 1-54, 401-532, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, 3388-3430, or 3549-3552;
   ii. 2′-OMe modified nucleotides at nucleotides 1, 2, and 3 of the guide region;
   iii. 2′-OMe modified nucleotides at nucleotides 1, 2, and 3 of the guide region and PS linkages between nucleotides 1 and 2, 2 and 3, and 3 and 4 of the guide region;
   iv. 2′-OMe modified nucleotides at nucleotides 1, 2, 3, 4, and 5 of the guide region;
   v. 2′-OMe modified nucleotides at nucleotides 1, 2, 3, 4, and 5 of the guide region and PS linkages between nucleotides 1 and 2, 2 and 3, 3 and 4, 4 and 5, and 5 and 6 of the guide region;
   vi. 2′O-moe modified nucleotides at nucleotides 1, 2, and 3 of the guide region;
   vii. 2′O-moe modified nucleotides at nucleotides 1, 2, and 3 of the guide region and PS linkages between nucleotides 1 and 2, 2 and 3, and 3 and 4 of the guide region;
   viii. an inverted abasic modified nucleotide at nucleotide 1 of the guide region;
   ix. an inverted abasic modified nucleotide at nucleotide 1 of the guide region and 2′-OMe modified nucleotides at nucleotides 1, 2, and 3 of the guide region; and
   x. an inverted abasic modified nucleotide at nucleotide 1 of the guide region, 2′-OMe modified nucleotides at nucleotides 1, 2, and 3 of the guide region, and PS linkages between nucleotides 1 and 2, 2 and 3, 3 and 4, 4 and 5, and 5 and 6 of the variable region.
Embodiment 356 is the gRNA of any one of embodiments 161-355, wherein the gRNA is an sgRNA and the upper stem region comprises at least one modification.
Embodiment 357 is the gRNA of embodiment 346, wherein the upper stem modification comprises any one or more of the following:
   i. a modification to any one or more of US1-US12 in the upper stem region;
   ii. a modification of at least 1, 2, 3, 4, 5, 6, 7, 8, 9, 10, 11, or all 12 nucleotides in the upper stem region; and
   iii. a modification of about 1-2, 1-3, 1-4, 1-5, 1-6, 1-7, 1-8, 1-9, 1-10, or 1-12 nucleotides in the upper stem region.
Embodiment 358 is the gRNA of any one of embodiments 356-357, wherein the upper stem modification comprises one or more of:
   i. a 2′-O-Me modified nucleotide;
   ii. a 2′-H modified nucleotide;
   iii. a 2′-F modified nucleotide; and
   iv. combinations thereof.
Embodiment 359 is the gRNA of any one of embodiments 161-358, wherein the gRNA is an sgRNA comprising one or more modifications in the hairpin 1 region.
Embodiment 360 is the gRNA of embodiment 359 wherein the sgRNA comprises a modification at H1-1.
Embodiment 361 is the gRNA of any one of embodiments 161-360, wherein the gRNA is an sgRNA comprising one or more modifications in the hairpin 2 region.
Embodiment 362 is the gRNA of embodiment 361, wherein the sgRNA comprises a modification at H2-1.
Embodiment 363 is the gRNA of any one of embodiments 161-362, wherein the gRNA is an sgRNA comprising comprises modifications at H1-1 to H1-12.
Embodiment 364 is the gRNA of any one of embodiments 161-363, wherein the gRNA is an sgRNA comprising comprises modifications at H2-1 to H2-15.
Embodiment 365 is the gRNA of any one of embodiments 161-364, wherein the gRNA is an sgRNA comprising one or more modifications in each of the upper stem region, the hairpin 1 region, and the hairpin 2 region.
Embodiment 366 is the gRNA of any one of embodiments 161-365, wherein the gRNA is an sgRNA comprising a modified nucleotide between hairpin 1 and hairpin 2 regions.
Embodiment 367 is the gRNA of any one of embodiments 161-366, which is an sgRNA further comprising a lower stem region comprising a modification.
Embodiment 368 is the gRNA of any one of embodiments 161-367, further comprising a 3′ end modification.
Embodiment 369 is the gRNA of embodiment 368, wherein at least two of the last four nucleotides at the 3′ end of the 3′ terminus are modified.
Embodiment 370 is the gRNA of embodiment 369, wherein at least two of the last four nucleotides at the 3′ end of the 3′ terminus are modified with 2′-O-Me, 2′-F, or 2′-O-moe.
Embodiment 371 is the gRNA of any one of embodiments 368-370, further comprising phosphorothioate (PS) bonds between one or more of the last four nucleotides at the 3′ end of the 3′ terminus.
Embodiment 372 is the gRNA of any one of embodiments 161-371, which is an sgRNA further comprising a bulge region comprising a modification.
Embodiment 373 is the gRNA of any one of embodiments 161-372, which is an sgRNA further comprising a nexus region comprising a modification.
Embodiment 374 is an sgRNA comprising any of SEQ ID Nos: 401-535, 601, 607-732, 801, 807-932, 1001, or 1007-1132, including the modifications of Table 1.
Embodiment 375 is an sgRNA comprising nucleic acids having at least 99, 98, 97, 96, 95, 94, 93, 92, 91, 90, 85, 80, 75, or 70% identity to the nucleic acids of any one of SEQ ID Nos: 401-535, 601, 607-732, 801, 807-932, 1001, or 1007-1132, wherein the modification at each nucleotide of the sgRNA that corresponds to a nucleotide of the reference sequence identifier in Table 1, is identical to or equivalent to the modification shown in the reference sequence identifier in Table 1.
Embodiment 376 is the gRNA of any one of embodiments 161-375, wherein the modification reduces gRNA degradation without significantly altering the ability of the guide to cleave a target nucleic acid.
Embodiment 377 is the gRNA of any one of embodiments 161-376, comprising a YA modification wherein the modification comprises 2′-fluoro, 2′-H, 2′-O-Me, ENA, UNA, or PS.
Embodiment 378 is the gRNA of any one of embodiments 161-377, comprising a YA modification wherein the modification alters the structure of the dinucleotide motif to reduce RNA endonuclease activity.
Embodiment 379 is the gRNA of any one of embodiments 161-378, comprising a YA modification wherein the modification interferes with recognition or cleavage of a YA site by an RNase and/or stabilizes an RNA structure.
Embodiment 380 is the gRNA of any one of embodiments 161-379, comprising a YA modification wherein the modification comprises one or more of:
   a. a ribose modification selected from 2′-O-alkyl, 2′-F, 2′-moe, 2′-F arabinose, and 2′-H (deoxyribose);
   b. a bicyclic ribose analog, such as LNA, BNA, and ENA;
   c. an unlocked nucleic acid modification;
   d. a base modification, such as inosine, pseudouridine, and 5′-methylcytosine; and
   e. an internucleoside linkage modification such as phosphorothioate.
Embodiment 381 is the gRNA of any one of the preceding embodiments, wherein the gRNA comprises a guide region that comprises a modification at nucleotide 5, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.
Embodiment 382 is the gRNA of any one of the preceding embodiments, wherein the gRNA comprises a guide region that comprises a modification at nucleotide 12, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.
Embodiment 383 is the gRNA of any one of the preceding embodiments, wherein the gRNA comprises a guide region that comprises a 2′-OMe modification at nucleotide 5 and/or nucleotide 12, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.
Embodiment 384 is the gRNA of any one of the preceding embodiments, wherein the gRNA comprises a guide region that comprises a 2′-F modification at nucleotide 5 and/or nucleotide 12, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.
Embodiment 385 is the gRNA of any one of the preceding embodiments, wherein the gRNA comprises a guide region that comprises a 2′-H modification at nucleotide 5 and/or nucleotide 12, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.
Embodiment 386 is the gRNA of any one of the preceding embodiments, wherein the gRNA comprises a guide region that comprises a phosphorothioate modification at nucleotide 5 and/or nucleotide 12, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.
Embodiment 387 is the gRNA of any one of the preceding embodiments, wherein the gRNA comprises a guide region that comprises modifications at:
   i. nucleotides 8-10;
   ii. nucleotides 8 and 9;
   iii. nucleotides 8 and 10; or
   iv. nucleotides 9 and 10,
   optionally wherein the guide region comprises 2′-OMe modifications at nucleotides 1-4, phosphorothioate modifications at nucleotides 1-3 and 6-7, and/or 2′-F modifications at nucleotides 11, 13, 14, 17, and 18.
Embodiment 388 is the gRNA of any one of the preceding embodiments, wherein the gRNA comprises a guide region that comprises 2′-F modifications at:
   i. nucleotides 8-10;
   ii. nucleotides 8 and 9;
   iii. nucleotides 8 and 10;
   iv. nucleotides 9 and 10; or
   v. nucleotide 8;
   optionally wherein the guide region comprises 2′-OMe modifications at nucleotides 1-4, phosphorothioate modifications at nucleotides 1-3 and 6-7, and/or 2′-F modifications at nucleotides 11, 13, 14, 17, and 18.
Embodiment 389 is the gRNA of any one of the preceding embodiments, wherein the gRNA comprises a guide region that comprises 2′-F modifications at:
   i. nucleotides 8-10;
   ii. nucleotides 8 and 9;
   iii. nucleotides 8 and 10;
   iv. nucleotides 9 and 10; or
   v. nucleotide 8;
   wherein nucleotides 8-10 do not comprise phosphorothioate modifications, and optionally wherein the guide region comprises 2′-OMe modifications at nucleotides 1-4, phosphorothioate modifications at nucleotides 1-3 and 6-7, and/or 2′-F modifications at nucleotides 11, 13, 14, 17, and 18.
Embodiment 390 is the gRNA of any one of the preceding embodiments, wherein the gRNA comprises a guide region that comprises 2′-F modifications at nucleotides 8-10 and:
   i. phosphorothioate modifications at 1, 2, or 3 of nucleotides 8-10;
   ii. a phosphorothioate modification at nucleotide 8;
   iii. a phosphorothioate modification at nucleotide 9;
   iv. a phosphorothioate modification at nucleotide 10;
   v. a phosphorothioate modification at nucleotides 8 and 9;
   vi. a phosphorothioate modification at nucleotides 8 and 10;
   vii. a phosphorothioate modification at nucleotides 9 and 10; or
   vii. a phosphorothioate modification at nucleotides 8-10
   optionally wherein the guide region comprises 2′-OMe modifications at nucleotides 1-4, phosphorothioate modifications at nucleotides 1-3 and 6-7, and/or 2′-F modifications at nucleotides 11, 13, 14, 17, and 18.
Embodiment 391 is the gRNA of any one of the preceding embodiments, wherein the gRNA comprises a guide region that comprises:
   i. a 2′-F or phosphorothioate modification at nucleotides 5 and 6;
   ii. a 2′-F modification at nucleotides 5 and 6;
   iii. a phosphorothioate modification at nucleotides 5 and 6;
   iv. a 2′-F modification at nucleotide 5 and a phosphorothioate modification at nucleotide 6; or
   v. a 2′-F modification at nucleotide 6 and a phosphorothioate modification at nucleotide 5;
   optionally wherein the guide region comprises 2′-OMe modifications at nucleotides 1-4, phosphorothioate modifications at nucleotides 1-3 and 7-10, and/or 2′-F modifications at nucleotides 8-11, 13, 14, 17, and 18.
Embodiment 392 is the gRNA of any one of the preceding embodiments, wherein the gRNA comprises a guide region that comprises 2′-F modifications at at least 1, 2, 3, 4, 5, or 6 of nucleotides 6-11, optionally wherein the guide region comprises 2′-OMe modifications at nucleotides 1-4, phosphorothioate modifications at nucleotides 1-3, and/or 2′-F modifications at nucleotides 13, 14, 17, and 18.
Embodiment 393 is the gRNA of any one of the preceding embodiments, wherein the gRNA comprises a guide region that comprises 2′-F modifications at at least 1, 2, 3, 4, 5, 6, 7, 8, 9, or 10 of nucleotides 1-4 and 6-11, optionally wherein the guide region comprises phosphorothioate modifications at nucleotides 1-3 and/or 2′-F modifications at nucleotides 13, 14, 17, and 18.
Embodiment 394 is the gRNA of any one of the preceding embodiments, wherein the gRNA comprises a guide region that comprises 2′-F modifications at nucleotides 6-11, optionally wherein the guide region comprises 2′-OMe modifications at nucleotides 1-4, phosphorothioate modifications at nucleotides 1-3, and/or 2′-F modifications at nucleotides 13, 14, 17, and 18.
Embodiment 395 is the gRNA of any one of the preceding embodiments, wherein the gRNA comprises a guide region that comprises 2′-F modifications at nucleotides 1-4, optionally wherein the guide region comprises phosphorothioate modifications at nucleotides 1-3 and 6-10, and/or 2′-F modifications at nucleotides 6-11, 13, 14, 17, and 18.
Embodiment 396 is the gRNA of any one of the preceding embodiments, wherein the gRNA comprises a guide region that comprises a 2′-F modification at nucleotide 9 and not a phosphorothioate modification at nucleotide 9, optionally wherein the guide region comprises 2′-OMe modifications at nucleotides 1-4, phosphorothioate modifications at nucleotides 1-3 and 6-8 and 10, and/or 2′-F modifications at nucleotides 8, 10, 11, 13, 14, 17, and 18.
Embodiment 397 is the gRNA of any one of the preceding embodiments, wherein the gRNA comprises a guide region that does not comprise 2′-F modifications at at least 1, 2, 3, 4, 5, 6, 7, or 8 of nucleotides 8-11, 13, 14, 17, and 18, optionally wherein the guide region comprises 2′-OMe modifications at nucleotides 1-4 and/or phosphorothioate modifications at nucleotides 1-3 and 6-10.
Embodiment 398 is the gRNA of any one of the preceding embodiments, wherein the gRNA comprises a guide region that does not comprise 2′-F modifications at nucleotides 8-11, 13, 14, 17, and 18, optionally wherein the guide region comprises 2′-OMe modifications at nucleotides 1-4 and/or phosphorothioate modifications at nucleotides 1-3 and 6-10.
Embodiment 399 is the gRNA of any one of the preceding embodiments, wherein the gRNA comprises a guide region that comprises 2′-OMe modifications at at least 1, 2, 3, or 4 of nucleotides 9, 11, 13, and 14, optionally wherein the guide region comprises 2′-OMe modifications at nucleotides 1-4 and/or phosphorothioate modifications at nucleotides 1-3 and 6-10.
Embodiment 400 is the gRNA of any one of the preceding embodiments, wherein the gRNA comprises a guide region that comprises 2′-OMe modifications at nucleotides 9, 11, 13, and 14, optionally wherein the guide region comprises 2′-OMe modifications at nucleotides 1-4 and/or phosphorothioate modifications at nucleotides 1-3 and 6-10.
Embodiment 401 is the gRNA of any one of the preceding embodiments, wherein the gRNA comprises a guide region that comprises phosphorothioate modifications at one or both of nucleotides 8 and 10, optionally wherein the guide region comprises 2′-OMe modifications at nucleotides 1-4, phosphorothioate modifications at nucleotides 1-3 and 6-7, and/or 2′-F modifications at nucleotides 8-11, 13, 14, 17, and 18.
Embodiment 402 is the gRNA of any one of the preceding embodiments, wherein the gRNA comprises a guide region that comprises modifications at at least 1, 2, 3, 4, 5, 6, 7, 8, 9, 10, 11, 12, 13, or all of the following nucleotides: 1, 2, 3, 4, 6, 7, 8, 9, 10, 11, 13, 14, 17, and 18, optionally wherein the modifications are 2′-OMe, 2′-fluoro, or phosphorothioate modifications.
Embodiment 403 is the gRNA of any one of the the preceding embodiments, wherein the gRNA comprises a guide region that comprises modifications at nucleotides 1, 2, 3, 4, 6, 7, 8, 9, 10, 11, 13, 14, 17, and 18, optionally wherein the modifications are 2′-OMe, 2′-fluoro, or phosphorothioate modifications.
Embodiment 404 is the gRNA of any one of the preceding embodiments, wherein 2′-OMe modifications are not present in the guide region at nucleotides 6-11 and 13-end.
Embodiment 405 is the gRNA of any one of the preceding embodiments, wherein 2′-fluoro modifications are not present in the guide region at nucleotides 1-7, 15, 16, and 19-end.
Embodiment 406 is the gRNA of any one of the preceding embodiments, wherein phosphorothioate modifications are not present in the guide region at nucleotides 4, 5, 11-14, 17, and 18.
Embodiment 407 is the gRNA of any one of the preceding embodiments, wherein the guide region comprises an unmodified nucleotide 20.
Embodiment 408 is the gRNA of any one of the preceding embodiments, wherein the guide region consists of 20 nucleotides.
Embodiment 409 is the gRNA of any one of the preceding embodiments, wherein the guide region comprises a YA site at nucleotides 5-6 and a modification at nucleotide 5.
Embodiment 410 is the gRNA of any one of the preceding embodiments, wherein the guide region comprises a YA site at nucleotides 12-13 and a modification at nucleotide 12.
Embodiment 411 is the gRNA of any one of the preceding embodiments, wherein the guide region comprises a YA site at nucleotides 15-16 and a modification at nucleotide 15.
Embodiment 412 is the gRNA of any one of the preceding embodiments, wherein the guide region comprises a YA site at nucleotides 16-17 and a modification at nucleotide 16.
Embodiment 413 is the gRNA of any one of the preceding embodiments, wherein the guide region comprises a YA site at nucleotides 19-20 and a modification at nucleotide 19.
Embodiment 414 is the gRNA of any one of the preceding embodiments, wherein the guide region does not comprise a YA site at nucleotides 5-6 and nucleotide 5 is unmodified.
Embodiment 415 is the gRNA of any one of the preceding embodiments, wherein the guide region does not comprise a YA site at nucleotides 12-13 and nucleotide 12 is unmodified.
Embodiment 416 is the gRNA of any one of the preceding embodiments, wherein the guide region does not comprise a YA site at nucleotides 15-16 and nucleotide 15 is unmodified.
Embodiment 417 is the gRNA of any one of the preceding embodiments, wherein the guide region does not comprise a YA site at nucleotides 16-17 and nucleotide 16 is unmodified.
Embodiment 418 is the gRNA of any one of the preceding embodiments, wherein the guide region does not comprise a YA site at nucleotides 19-20 and nucleotide 19 is unmodified.
Embodiment 419 is the gRNA of any one of the preceding embodiments, wherein the gRNA comprises a guide region that comprises at least 1, 2, 3, 4, 5, 6, 7, 8, 9, 10, 11, 12, 13, or all of the following:
   a. 2′-OMe and phosphorothioate modifications at nucleotide 1;
   b. 2′-OMe and phosphorothioate modifications at nucleotide 2;
   c. 2′-OMe and phosphorothioate modifications at nucleotide 3;
   d. a 2′-OMe modification at nucleotide 4;
   e. a phosphorothioate modification at nucleotide 6;
   f. a phosphorothioate modification at nucleotide 7;
   g. 2′-fluoro and phosphorothioate modifications at nucleotide 8;
   h. 2′-fluoro and phosphorothioate modifications at nucleotide 9;
   i. 2′-fluoro and phosphorothioate modifications at nucleotide 10;
   j. a 2′-fluoro modification at nucleotide 11;
   k. a 2′-fluoro modifications at nucleotide 13;
   l. a 2′-fluoro modifications at nucleotide 14;
   m. a 2′-fluoro modifications at nucleotide 17; and
   n. a 2′-fluoro modifications at nucleotide 18.
Embodiment 420 is the gRNA of any one of the preceding embodiments, wherein the guide region comprises each of the modifications set forth in the preceding embodiment.
Embodiment 421 is the gRNA of any one of the preceding embodiments, wherein the guide region comprises at least 1, 2, 3, or 4 of the following:
   i. a 2′-OMe modification at nucleotide 5 if nucleotides 5 and 6 form a YA site;
   ii. a 2′-OMe modification at nucleotide 12 if nucleotides 12 and 13 form a YA site;
   iii. a phosphorothioate modification at nucleotide 15 if nucleotides 15 and 16 form a YA site;
   iv. a phosphorothioate modification at nucleotide 16 if nucleotides 16 and 17 form a YA site; and
   v. a phosphorothioate or 2′-fluoro modification at nucleotide 19 if nucleotides 19 and 20 form a YA site.
Embodiment 422 is the gRNA of any one of the preceding embodiments, wherein the guide region comprises a YA site at nucleotides 5-6 and a a 2′-OMe modification at nucleotide 5.
Embodiment 423 is the gRNA of any one of the preceding embodiments, wherein the guide region comprises a YA site at nucleotides 12-13 and a 2′-OMe modification at nucleotide 12.
Embodiment 424 is the gRNA of any one of the preceding embodiments, wherein the guide region comprises a YA site at nucleotides 15-16 and a phosphorothioate modification at nucleotide 15.
Embodiment 425 is the gRNA of any one of the preceding embodiments, wherein the guide region comprises a YA site at nucleotides 16-17 and a phosphorothioate modification at nucleotide 16.
Embodiment 426 is the gRNA of any one of the preceding embodiments, wherein the guide region comprises a YA site at nucleotides 19-20 and a phosphorothioate modification at nucleotide 19.
Embodiment 427 is the gRNA of any one of the preceding embodiments, wherein the guide region comprises a 2′-fluoro modification at nucleotide 19.
Embodiment 428 is the gRNA of any one of the preceding embodiments, wherein the guide region comprises an unmodified nucleotide 15 or only a phosphorothioate modification at nucleotide 15.
Embodiment 429 is the gRNA of any one of the preceding embodiments, wherein the guide region comprises an unmodified nucleotide 16 or only a phosphorothioate modification at nucleotide 16.
Embodiment 430 is the gRNA of any one of the preceding embodiments, wherein the gRNA is an sgRNA comprising a conserved portion of an sgRNA comprising a hairpin region, wherein the hairpin region lacks at least 5-10 nucleotides.
Embodiment 431 is the gRNA of embodiment 430, wherein the at least 5-10 lacking nucleotides are consecutive.
Embodiment 432 is the gRNA of embodiment 430 or 431, wherein the at least 5-10 lacking nucleotides:
   i. are within hairpin 1;
   ii. are within hairpin 1 and the “N” between hairpin 1 and hairpin 2;
   iii. are within hairpin 1 and the two nucleotides immediately 3′ of hairpin 1;
   iv. include at least a portion of hairpin 1;
   v. are within hairpin 2;
   vi. include at least a portion of hairpin 2;
   vii. are within hairpin 1 and hairpin 2;
   viii. include at least a portion of hairpin 1 and include the “N” between hairpin 1 and hairpin 2;
   ix. include at least a portion of hairpin 2 and include the “N” between hairpin 1 and hairpin 2;
   x. include at least a portion of hairpin 1, include the “N” between hairpin 1 and hairpin 2, and include at least a portion of hairpin 2;
   xi. are within hairpin 1 or hairpin 2, optionally including the “N” between hairpin 1 and hairpin 2;
   xii. are consecutive;
   xiii. are consecutive and include the “N” between hairpin 1 and hairpin 2;
   xiv. are consecutive and span at least a portion of hairpin 1 and a portion of hairpin 2;
   xv. are consecutive and span at least a portion of hairpin 1 and the “N” between hairpin 1 and hairpin 2; or
   xvi. are consecutive and span at least a portion of hairpin 1 and two nucleotides immediately 3′ of hairpin 1.
Embodiment 433 is the gRNA of any one of embodiments 430-432, wherein the at least 5-10 nucleotides comprise nucleotides 54-61 of SEQ ID NO:400, nucleotides 53-60 of SEQ ID NO:400; or nucleotides 54-58 of SEQ ID NO:400, optionally wherein the sgRNA comprises modifications at least H1-1 to H1-5 and H2-1 to H2-12.
Embodiment 434 is the gRNA of any one of embodiments 430-433, wherein the at least 5-10 nucleotides:
   i. consist of 5-10 nucleotides;
   ii. consist of 6-10 nucleotides;
   iii. consist of 5 nucleotides;
   iv. consist of 6 nucleotides;
   v. consist of 7 nucleotides;
   vi. consist of 8 nucleotides;
   vii. consist of 9 nucleotides;
   viii. consist of 10 nucleotides;
   ix. consist of 5-10 consecutive nucleotides;
   x. consist of 6-10 consecutive nucleotides;
   xi. consist of 5 consecutive nucleotides;
   xii. consist of 6 consecutive nucleotides;
   xiii. consist of 7 consecutive nucleotides;
   xiv. consist of 8 consecutive nucleotides;
   xv. consist of 9 consecutive nucleotides; or
   xvi. consist of 10 consecutive nucleotides.
Embodiment 435 is the gRNA of embodiment 434, wherein the at least 5-10 nucleotides comprise nucleotides 54-61 of SEQ ID NO:400, nucleotides 53-60 of SEQ ID NO:400; or nucleotides 54-58 of SEQ ID NO:400, optionally wherein the sgRNA comprises modifications at least H1-1 to H1-5 and H2-1 to H2-12.
Embodiment 436 is the gRNA of any one of embodiments 430-435, wherein the at least 5-10 nucleotides:
   i. comprise nucleotides 54-61 of SEQ ID NO:400;
   ii. comprise nucleotides 53-60 of SEQ ID NO:400;
   iii. comprise nucleotides 54-58 of SEQ ID NO:400.
   iv. consist of nucleotides 54-61 of SEQ ID NO:400;
   v. consist of nucleotides 53-60 of SEQ ID NO:400; or
   vi. consist of nucleotides 54-58 of SEQ ID NO:400.
Embodiment 437 is the gRNA of any one of the preceding embodiments, wherein the gRNA comprises modifications and/or unmodified nucleotides at at least 15 of nucleotides 1-20 from the 5′ end of the 5′ terminus that match the modification pattern at nucleotides 1-20 of any one of the gRNAs of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.
Embodiment 438 is the gRNA of any one of the preceding embodiments, wherein the gRNA comprises modifications and/or unmodified nucleotides at at least 16 of nucleotides 1-20 from the 5′ end of the 5′ terminus that match the modification pattern at nucleotides 1-20 of any one of the gRNAs of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.
Embodiment 439 is the gRNA of any one of the preceding embodiments, wherein the gRNA comprises modifications and/or unmodified nucleotides at at least 17 of nucleotides 1-20 from the 5′ end of the 5′ terminus that match the modification pattern at nucleotides 1-20 of a gRNA, wherein the gRNA is any one of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.
Embodiment 440 is the gRNA of any one of the preceding embodiments, wherein the gRNA comprises modifications and/or unmodified nucleotides at at least 18 of nucleotides 1-20 from the 5′ end of the 5′ terminus that match the modification pattern at nucleotides 1-20 of any one of the gRNAs of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.
Embodiment 441 is the gRNA of any one of the preceding embodiments, wherein the gRNA comprises modifications and/or unmodified nucleotides at at least 19 of nucleotides 1-20 from the 5′ end of the 5′ terminus that match the modification pattern at nucleotides 1-20 of any one of the gRNAs of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.
Embodiment 442 is the gRNA of any one of the preceding embodiments, wherein the gRNA comprises modifications and/or unmodified nucleotides at nucleotides 1-20 from the 5′ end of the 5′ terminus that match the modification pattern at nucleotides 1-20 of any one of the gRNAs of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.
Embodiment 443 is the gRNA of any one of the preceding embodiments, wherein the gRNA comprises a modification pattern that matches at least 75% of the modification pattern of any one of the gRNAs of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.
Embodiment 444 is the gRNA of any one of the preceding embodiments, wherein the gRNA comprises the modification pattern of any one of the gRNAs in Table 1, wherein the modification pattern is the same as any one of the gRNAs of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.
Embodiment 445 is the gRNA of any one of embodiments 437-444, further comprising a sequence having at least 75% identity to the sequence of nucleotides 21-end of the gRNA.
Embodiment 446 is the gRNA of any one of embodiments 437-444, further comprising a sequence having at least 80% identity to the sequence of nucleotides 21-end of the gRNA.
Embodiment 447 is the gRNA of any one of embodiments 437-444, further comprising a sequence having at least 85% identity to the sequence of nucleotides 21-end of the gRNA.
Embodiment 448 is the gRNA of any one of embodiments 437-444, further comprising a sequence having at least 90% identity to the sequence of nucleotides 21-end of the gRNA.
Embodiment 449 is the gRNA of any one of embodiments 437-444, further comprising a sequence having at least 95% identity to the sequence of nucleotides 21-end of the gRNA.
Embodiment 450 is the gRNA of any one of embodiments 437-444, further comprising a sequence having at least 98% identity to the sequence of nucleotides 21-end of the gRNA.
Embodiment 451 is the gRNA of any one of embodiments 437-444, further comprising a sequence having 100% identity to the sequence of nucleotides 21-end of the gRNA.
Embodiment 452 is an LNP composition comprising a gRNA of any one of the preceding embodiments.
Embodiment 453 is a composition comprising a gRNA of any one of embodiments 1-451 associated with a lipid nanoparticle (LNP).
Embodiment 454 is a composition comprising the gRNA of any one of embodiments 1-451, or the composition of any one of embodiments 452-453, further comprising a nuclease or an mRNA which encodes the nuclease.
Embodiment 455 is the composition of embodiment 454, wherein the nuclease is a Cas protein.
Embodiment 456 is the composition of embodiment 455, wherein the Cas protein is a Cas9.
Embodiment 457 is the composition of embodiment 456, wherein the Cas9 is an S. pyogenes Cas9 or an S. aureus Cas9.
Embodiment 458 is the composition of any one of embodiments 453-457, wherein the nuclease is a nickase or a dCas.
Embodiment 459 is the composition of any one of embodiments 453-458, wherein the nuclease is modified.
Embodiment 460 is the composition of embodiment 459 wherein the modified nuclease comprises a nuclear localization signal (NLS).
Embodiment 461 is the composition of any one of embodiments 452-460, comprising an mRNA which encodes the nuclease.
Embodiment 462 is the composition of embodiment 461, wherein the mRNA comprises the sequence of any one of SEQ ID NOs: 3499-3527 or 3529-3546.
Embodiment 463 is a pharmaceutical formulation comprising the gRNA of any one of embodiments 1-451 or the composition of any one of embodiments 452-462 and a pharmaceutically acceptable carrier.
Embodiment 464 is a method of modifying a target DNA comprising, delivering a Cas protein or a nucleic acid encoding a Cas protein, and any one or more of the following to a cell:
   i. the gRNA of any one of embodiments 1-451;
   ii. the composition of any one of embodiments 452-462; and
   iii. the pharmaceutical formulation of embodiment 463.
Embodiment 465 is the method of embodiment 464, wherein the method results in an insertion or deletion in a gene.
Embodiment 466 is the method of embodiment 464 or embodiment 465, further comprising delivering to the cell a template, wherein at least a part of the template incorporates into a target DNA at or near a double strand break site induced by the Cas protein.
Embodiment 467 is the gRNA of any one of embodiments 1-451, the composition of embodiments 452-462, or the pharmaceutical formulation of embodiment 463 for use in preparing a medicament for treating a disease or disorder.
Embodiment 468 is the use of the gRNA of any one of embodiments 1-451, the composition of embodiments 452-462, or the pharmaceutical formulation of embodiment 463 in the manufacture of a medicament for treating a disease or disorder.

### FIGURE LEGENDS

FIGS. 1A and 1B show in vivo % editing and serum TTR results, respectively, for the indicated guides.

FIGS. 2A and 2B show in vivo % editing and serum TTR results, respectively, for the indicated guides.

FIGS. 3A and 3B show in vivo % editing and serum TTR results, respectively, for the indicated guides. FIGS. 3C and 3D show in vivo % editing and serum TTR results, respectively, for the indicated guides. FIGS. 3E and 3F show in vivo % editing and serum TTR results, respectively, for the indicated guides in rats.

FIGS. 4A and 4B show in vivo % editing and serum TTR results, respectively, for the indicated guides.

FIG. 5 show % editing in neuro2A cells in vitro.

FIGS. 6A and 6B show in vivo % editing and serum TTR results, respectively, for the indicated guides.

FIGS. 7A and 7B show in vivo % editing and serum TTR results, respectively, for the indicated guides.

FIGS. 8A and 8B show in vivo % editing and serum TTR results, respectively, for the indicated guides. FIGS. 8C and 8D show in vivo % editing and serum TTR results, respectively, for the indicated guides.

FIGS. 9A, 9B, and 9C show % editing by concentration in PHH (9A), PCH (9B), and HepG2 (9C) cells, respectively, for the indicated guides.

FIG. 10A shows an exemplary sgRNA (SEQ ID NO: 801, methylation not shown) in a possible secondary structure with labels designating individual nucleotides of the conserved region of the sgRNA, including the lower stem, bulge, upper stem, nexus (the nucleotides of which can be referred to as N1 through N18, respectively, in the 5′ to 3′ direction), and the hairpin region which includes hairpin 1 and hairpin 2 regions. A nucleotide between hairpin 1 and hairpin 2 is labeled n. A guide region may be present on an sgRNA and is indicated in this figure as “(N)x” preceding the conserved region of the sgRNA.

FIG. 10B labels the 10 conserved region YA sites in an exemplary sgRNA sequence (SEQ ID NO: 801, methylation not shown) from 1 to 10. The numbers 25, 45, 50, 56, 64, 67, and 83 indicate the position of the pyrimidine of YA sites 1, 5, 6, 7, 8, 9, and 10 in an sgRNA with a guide region indicated as (N)ₓ, e.g., wherein x is optionally 20.

FIG. 11A-E show results of nuclease stability assays in which the indicated guides were incubated with 0.01 mg/mL human liver cytosol (HLC) and cleavage sites were determined. FLP indicates signal from full-length product.

FIG. 11F illustrates the location of cleavage sites observed in FIGS. 11A-E mapped onto an exemplary guide sequence and possible secondary structure of SEQ ID NO: 401 (not all modifications are shown). Open triangles show YA cleavage sites in the guide region. Closed triangles show YA cleavage sites in the conserved region.

FIGS. 12A-G show results of nuclease stability assays in which the indicated guides were incubated with 0.01 mg/mL human liver cytosol (HLC) and cleavage sites were determined.

FIGS. 13A-B show results of nuclease stability assays in which G010039 was incubated with 0.01 mg/mL (A) or 8.5 mg/mL (B) human liver cytosol (HLC).

FIG. 14 shows % editing results from experiments in which lipoplexes comprising the indicated guides were transfected into primary mouse hepatocytes (PMH).

FIG. 15A-C show % editing results from experiments in which lipoplexes comprising the indicated guides were transfected into PMH, primary cynomolgous macaque hepatocytes (PCH), or primary human hepatocytes (PHH), respectively.

FIG. 16A shows a scatter plot and correlation values for % editing results from experiments in which sgRNA was administered to mice in vivo or delivered to PMH via lipoplex transfection of the sgRNA.

FIGS. 16B-F show correlation of in vivo and in vitro % editing results in which the in vitro results were generated by delivering the sgRNAs in LNPs to PHH.

FIG. 16G shows a comparison of % editing with the indicated guides delivered to PMH by lipoplex transfection (data above left box), to PMH in LNP (data above center box), or to mice in vivo (data above right box).

FIG. 16H shows a comparison of % editing with the indicated guides delivered to PMH in LNP (1 ng, 3 ng, 10 ng) or to mice in vivo (0.1 mpk, 0.3 mpk).

FIG. 16I shows the results from FIG. 16G replotted to indicate differences in editing between G000282 and G000211. The barplot values were generated by dividing % editing of the G000282 value by the % editing of the G000211 value to indicate fold differences in editing. The indicated guides were delivered to PMH by lipoplex transfection (data above left box), to PMH in LNP (data above center box), or to mice in vivo (data above right box).

FIG. 16J shows the results from FIG. 16H replotted to indicate differences in editing between G000283 and G000269. The barplot values were generated by dividing % editing of the G000283 value by the % editing of the G000269 value to indicate fold differences in editing. The indicated guides were delivered to PMH in LNP (data above left box) or to mice in vivo (data above right box).

FIGS. 17A-B show in vivo % editing and serum TTR results, respectively, for the indicated guides.

FIGS. 18A-B show in vivo % editing and serum TTR results, respectively, for the indicated guides. FIGS. 18C-D show in vivo % editing and serum TTR results, respectively, for the indicated guides. FIGS. 18E-F show in vivo % editing and serum TTR results, respectively, for the indicated guides.

FIGS. 19A-B show in vivo % editing and serum TTR results, respectively, for the indicated guides. FIGS. 19C-D show in vivo % editing and serum TTR results, respectively, for the indicated guides.

FIGS. 20A-B show in vivo % editing and serum TTR results, respectively, for the indicated guides at the indicated concentrations. FIGS. 20C-D show in vivo % editing and serum TTR results, respectively, for the indicated guides at the indicated concentrations. FIGS. 20E-F show in vivo % editing and serum TTR results, respectively, for the indicated guides at the indicated concentrations.

FIGS. 21A-B show in vivo % editing and serum TTR results, respectively, for the indicated guides.

FIGS. 22A-B show in vivo % editing and serum TTR results, respectively, for the indicated guides.

FIGS. 23A-B show editing frequency for the indicated guides.

FIGS. 24A-B show in vivo % editing and serum TTR results, respectively, for the indicated guides.

FIGS. 25A-E show indel frequency versus guide concentration for the indicated guides.

FIGS. 26A-E show indel frequency versus guide concentration for the indicated guides.

FIGS. 27A-D show indel frequency versus guide concentration for the indicated guides.

FIGS. 28A-D show indel frequency versus guide concentration for the indicated guides.

FIGS. 29A-B and 29F show editing frequency for guides with the indicated dinucleotide modification (for a given 5′ modified position, the immediately subsequent position was also modified in the same way). FIGS. 29C-E show editing frequency for guides with the indicated modification at an individual nucleotide.

FIGS. 30A-C show influence scores for the indicated modification at guide positions 1-20.

FIGS. 31A-C show editing frequency for the indicated guides. Guides are grouped into boxes based on having similar conserved region modification patterns.

### DETAILED DESCRIPTION

Provided herein are modified guide RNAs (gRNAs) for use in gene editing methods. Sequences of engineered and tested gRNAs are shown in Table 1.

Certain of the gRNAs provided herein are modified dual guide RNAs (dgRNAs) for use in gene editing methods. Sequences of engineered and tested dgRNAs are shown in Table 1. Certain of the dgRNAs have certain modifications at YA sites in the dgRNA, including modifications in the crRNA and/or the trRNA.

Certain of the gRNAs provided herein are modified single guide RNAs (sgRNAs) for use in gene editing methods. Sequences of engineered and tested sgRNAs are shown in Table 1. Certain of the sgRNAs have certain modifications at YA sites in the sgRNA, including modifications in the crRNA portion of the sgRNA and/or the trRNA portion of the sgRNA.

Also provided herein are short-single guide RNAs (short-sgRNAs), optionally modified, for use in gene editing methods. Sequences of engineered and tested short-sgRNAs are shown in Table 1. Certain of the short-sgRNAs have certain modifications at YA sites in the short-sgRNA, including modifications in the crRNA portion of the short-sgRNA and/or the trRNA portion of the short-sgRNA.

This disclosure further provides uses of these gRNAs (e.g., sgRNA, short-sgRNA, dgRNA, or crRNA) to alter the genome of a target nucleic acid in vitro (e.g., cells cultured in vitro for use in ex vivo therapy or other uses of genetically edited cells) or in a cell in a subject such as a human (e.g., for use in in vivo therapy). The present disclosure also provides methods for preventing or treating a disease in a subject by modifying a target gene associated with a disease. The disclosed gRNAs can be used with any cell type and at any genetic locus amenable to nuclease mediated genome editing technology.

Lengthy table referenced here
US20210087568A1-20210325-T00001
Please refer to the end of the specification for access instructions.

Nucleotide modifications are indicated in Table 1 as follows: m: 2′-OMe; *: PS linkage; f: 2′-fluoro; (invd): inverted abasic; moe: 2′-moe; e: ENA; d: deoxyribonucleotide (also note that T is always a deoxyribonucleotide); x: UNA. Thus, for example, mA represents 2′-O-methyl adenosine; xA represents a UNA nucleotide with an adenine nucleobase; eA represents an ENA nucleotide with an adenine nucleobase; and dA represents an adenosine deoxyribonucleotide.

sgRNA designations are sometimes provided with one or more leading zeroes immediately following the G. This does not affect the meaning of the designation. Thus, for example, G000282, G0282, G00282, and G282 refer to the same sgRNA. Similarly, crRNA and or trRNA designations are sometimes provided with one or more leading zeroes immediately following the CR or TR, respectively, which does not affect the meaning of the designation. Thus, for example, CR000100, CR00100, CR0100, and CR100 refer to the same crRNA, and TR000200, TR00200, TR0200, and TR200 refer to the same trRNA.

For SEQ ID NOs: 401-535, 1001, and 1007-1032, positions correspond to sgRNA regions as follows: 1-20, guide region; 21-26 and 45-50, lower stem; 27-28 and 41-44, bulge; 29-40, upper stem (of which 33-36 are a tetraloop); 51-68, nexus; 69-80, hairpin 1; 82-96, hairpin 2 (position 81 is a nucleotide between hairpin 1 and hairpin 2); 97-100, 3′ terminus region.

For SEQ ID NOs 601 and 607-732, no guide region is shown and the positions corresponding to the remaining regions are each decremented by 20 relative to those given for SEQ ID NOs: 401-532. For SEQ ID NOs 801 and 807-932, the spacer is the length of x and the positions corresponding to the remaining regions are each decremented by 20 and incremented by x relative to those given for SEQ ID NOs: 401-532.

### Definitions

“Editing efficiency” or “editing percentage” or “percent editing” as used herein is the total number of sequence reads with insertions or deletions of nucleotides into the target region of interest over the total number of sequence reads following cleavage by a Cas RNP.

“Regions” as used herein describes conserved groups of nucleic acids. Regions may also be referred to as “modules” or “domains.” Regions of an sgRNA may perform particular functions, e.g., in directing endonuclease activity of the RNP, for example as described in Briner A E et al., Molecular Cell 56:333-339 (2014). Exemplary regions of an sgRNA are described in Table 3.

“Hairpin” as used herein describes a duplex of nucleic acids that is created when a nucleic acid strand folds and forms base pairs with another section of the same strand. A hairpin may form a structure that comprises a loop or a U-shape. In some embodiments, a hairpin may be comprised of an RNA loop. Hairpins can be formed with two complementary sequences in a single nucleic acid molecule bind together, with a folding or wrinkling of the molecule. In some embodiments, hairpins comprise stem or stem loop structures. As used herein, a “hairpin region” refers to hairpin 1 and hairpin 2 and the “n” between hairpin 1 and hairpin 2 of a conserved portion of an sgRNA.

“Ribonucleoprotein” (RNP) or “RNP complex” as used herein describes an sgRNA, for example, together with a nuclease, such as a Cas protein. In some embodiments, the RNP comprises Cas9 and gRNA (e.g., sgRNA, short-sgRNA, dgRNA, or crRNA).

“Stem loop” as used herein describes a secondary structure of nucleotides that form a base-paired “stem” that ends in a loop of unpaired nucleic acids. A stem may be formed when two regions of the same nucleic acid strand are at least partially complementary in sequence when read in opposite directions. “Loop” as used herein describes a region of nucleotides that do not base pair (i.e., are not complementary) that may cap a stem. A “tetraloop” describes a loop of 4 nucleotides. As used herein, the upper stem of an sgRNA may comprise a tetraloop.

“Guide RNA”, “gRNA”, and “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences. Guide RNAs can include modified RNAs as described herein.

In some embodiments, the gRNA (e.g., sgRNA) comprises a “guide region”, which is sometimes referred to as a “spacer” or “spacer region,” for example, in Briner A E et al., Molecular Cell 56:333-339 (2014) for sgRNA (but applicable herein to all guide RNAs). The guide region or spacer region is also sometimes referred to as a “variable region,” “guide domain” or “targeting domain.” In some embodiments, a “guide region” immediately precedes a “conserved portion of an sgRNA” at its 5′ end, and in some embodiments the sgRNA is a short-sgRNA. An exemplary “conserved portion of an sgRNA” is shown in Table 2. In some embodiments, a “guide region” comprises a series of nucleotides at the 5′ end of a crRNA. In some embodiments, the guide region comprises one or more YA sites (“guide region YA sites”). In some embodiments, the guide region comprises one or more YA sites located at positions from a given nucleotide relative to the 5′ end to the end of the guide region. Such ranges of positions are referred to as, e.g., “5-end, 6-end, 7-end, 8-end, 9-end, or 10-end from the 5′ end of the 5′ terminus” where the “end” in “5-end”, etc., refers to most 3′ nucleotide in the guide region. (Similarly, expressions such as “nucleotides 21-end of the gRNA” refer to the range from nucleotide 21 from the 5′ end of the 5′ terminus of the gRNA to the final nucleotide at the 3′ end of the gRNA.) Furthermore, a nucleotide that is, for example, 6 nucleotides from the 5′ end of a particular sgRNA segment is the sixth nucleotide of that segment, or “nucleotide 6” from the 5′ end, e.g., , where N is the 6^{th} nucleotide from the 5′ end. A range of nucleotides that is located “at or after” 6 nucleotides from the 5′ end begins with the 6^{th} nucleotide and continues down the chain toward the 3′ end. Similarly, a nucleotide that is, for example, 5 nucleotides from the 3′ end of the chain is the 5^{th} nucleotide when counting from the 3′ end of the chain, e.g. NXXXX. A numeric position or range in the guide region refers to the position as determined from the 5′ end unless another point of reference is specified; for example, “nucleotide 5” in a guide region is the 5^{th} nucleotide from the 5′ end.

In some embodiments, a gRNA comprises nucleotides that “match the modification pattern” at corresponding or specified nucleotides of a gRNA described herein. This means that the nucleotides matching the modification pattern have the same modifications (e.g., phosphorothioate, 2′-fluoro, 2′-OMe, etc.) as the nucleotides at the corresponding positions of the gRNA described herein, regardless of whether the nucleobases at those positions match. For example, if in a first gRNA, nucleotides 5 and 6, respectively, have 2′-OMe and phosphorothioate modifications, then this gRNA has the same modification pattern at nucleotides 5 and 6 as a second gRNA that also has 2′-OMe and phosphorothioate modifications at nucleotides 5 and 6, respectively, regardless of whether the nucleobases at positions 5 and 6 are the same or different in the first and second gRNAs. However, a 2′-OMe modification at nucleotide 6 but not nucleotide 7 is not the same modification pattern at nucleotides 6 and 7 as a 2′-OMe modification at nucleotide 7 but not nucleotide 6. Similarly, a modification pattern that matches at least 75% of the modification pattern of a gRNA described herein means that at least 75% of the nucleotides have the same modifications as the corresponding positions of the gRNA described herein. Corresponding positions may be determined by pairwise or structural alignment.

A “conserved region” of a S. pyogenes Cas9 (“spyCas9” (also referred to as “spCas9”)) sgRNA” is shown in Table 2. The first row shows the numbering of the nucleotides; the second row shows the sequence (e.g., SEQ ID NO: 400); and the third row shows the regions.

As used herein, a “short-single guide RNA” (“short-sgRNA”) is an sgRNA comprising a conserved portion of an sgRNA comprising a hairpin region, wherein the hairpin region lacks at least 5-10 or 6-10 nucleotides. In some embodiments, a short-sgRNA lacks at least nucleotides 54-58 (AAAAA) of the conserved portion of a S. pyogenes Cas9 (“spyCas9”) sgRNA, as shown in Table 2. In some embodiments, a short-sgRNA is a non-spyCas9 sgRNA that lacks nucleotides corresponding to nucleotides 54-58 (AAAAA) of the conserved portion of a spyCas9 as determined, for example, by pairwise or structural alignment. In some embodiments, a short-sgRNA lacks at least nucleotides 54-61 (AAAAAGUG) of the conserved portion of a spyCas9 sgRNA. In some embodiments, a short-sgRNA lacks at least nucleotides 53-60 (GAAAAAGU) of the conserved portion of a spyCas9 sgRNA. In some embodiments, a short-sgRNA lacks 4, 5, 6, 7, or 8 nucleotides of nucleotides 53-60 (GAAAAAGU) or nucleotides 54-61 (AAAAAGUG) of the conserved portion of a spyCas9 sgRNA, or the corresponding nucleotides of the conserved portion of a non-spyCas9 sgRNA as determined, for example, by pairwise or structural alignment.

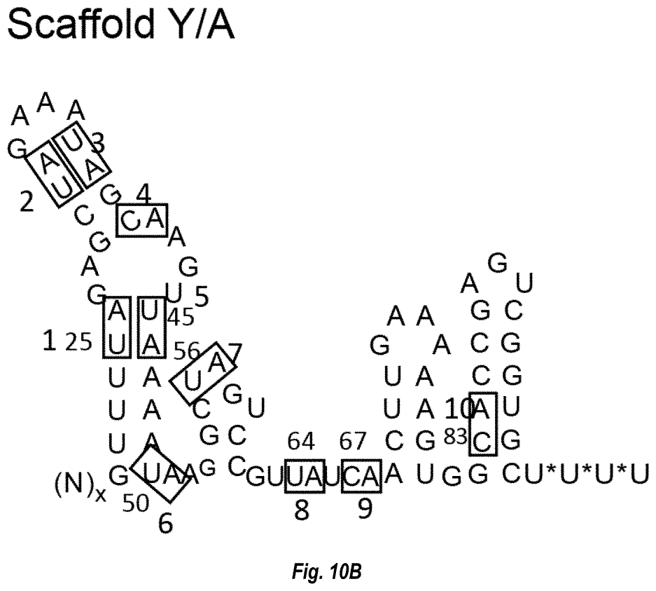

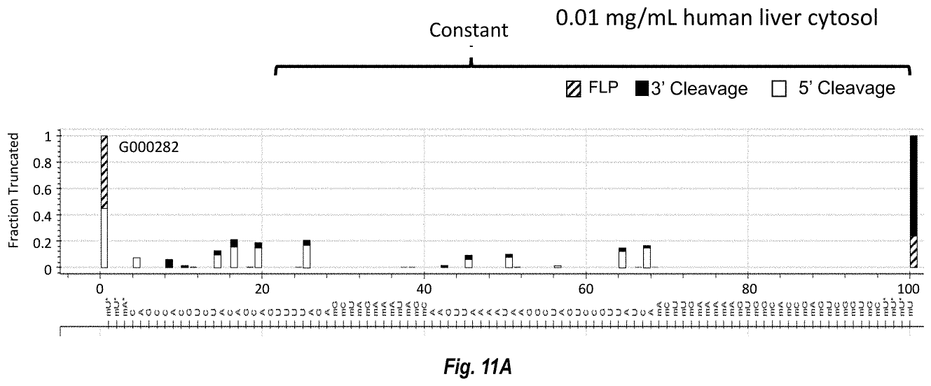

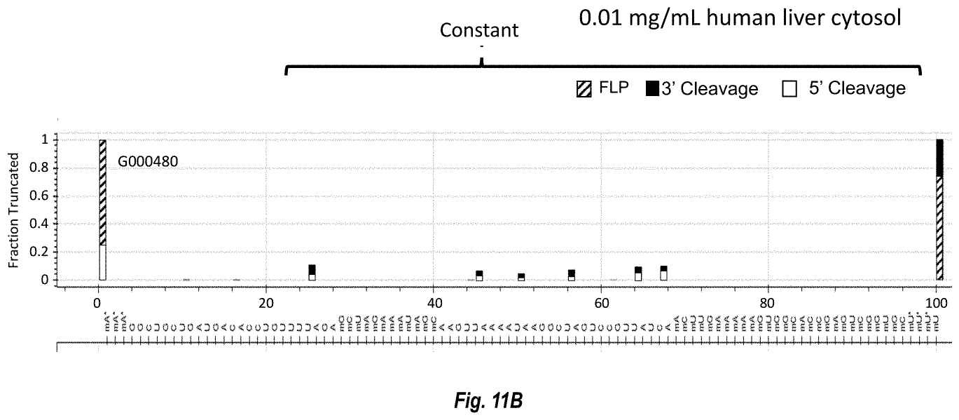

As used herein, a “YA site” refers to a 5′-pyrimidine-adenine-3′ dinucleotide. For clarification, a “YA site” in an original sequence that is altered by modifying a base is still considered a (modified) YA site in the resulting sequence, regardless of the absence of a literal YA dinucleotide. A “conserved region YA site” is present in the conserved region of an sgRNA. A “guide region YA site” is present in the guide region of an sgRNA. An unmodified YA site in an sgRNA may be susceptible to cleavage by RNase-A like endonucleases, e.g., RNase A. In some embodiments, a short-sgRNA comprises about 10 YA sites in its conserved region. In some embodiments, an sgRNA comprises 1, 2, 3, 4, 5, 6, 7, 8, 9, or 10 YA sites in its conserved region. Exemplary conserved region YA sites are indicated in FIG. 10B. Exemplary guide region YA sites are not shown in FIG. 10B, as the guide region may be any sequence, including any number of YA sites. In some embodiments, an sgRNA comprises 1, 2, 3, 4, 5, 6, 7, 8, 9, or 10 of the YA sites indicated in FIG. 10B. In some embodiments, an sgRNA comprises 1, 2, 3, 4, 5, 6, 7, 8, 9, or 10 YA sites at the following positions or a subset thereof: LS5-LS6; US3-US4; US9-US10; US12-B3; LS7-LS8; LS12-N1; N6-N7; N14-N15; N17-N18; and H2-2 to H2-3. In some embodiments, a YA site comprises a modification, meaning that at least one nucleotide of the YA site is modified. In some embodiments, the pyrimidine (also called the pyrimidine position) of the YA site comprises a modification (which includes a modification altering the internucleoside linkage immediately 3′ of the sugar of the pyrimidine). In some embodiments, the adenine (also called the adenine position) of the YA site comprises a modification (which includes a modification altering the internucleoside linkage immediately 3′ of the sugar of the adenine). In some embodiments, the pyrimidine position and the adenine position of the YA site comprise modifications. In some embodiments, a short-sgRNA guide region or short-sgRNA conserved region described herein comprises one or more YA sites (“guide region YA sites” or “conserved region YA sites”). In some embodiments, a crRNA or a trRNA described herein comprises one or more YA sites.

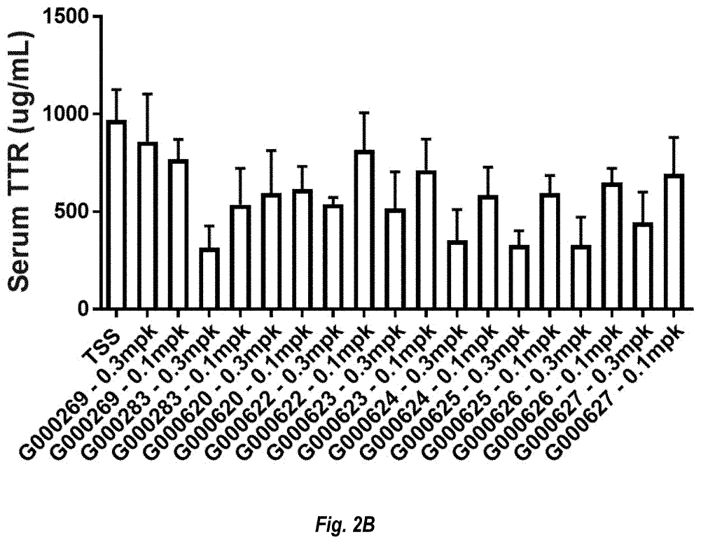

As discussed herein, positions of nucleotides corresponding to those described with respect to spyCas9 gRNA can be identified in another gRNA with sequence and/or structural similarity by pairwise or structural alignment. Structural alignment is useful where molecules share similar structures despite considerable sequence variation. For example, spyCas9 and Staphylococcus aureus Cas9 (“SaCas9”) have divergent sequences, but significant structural alignment. See, e.g., FIG. 2(F) from Nishimasu et al., Cell 162(5): 1113-1126 (2015). Structural alignment can be used to identify nucleotides in a saCas9 or other sgRNA that correspond to particular positions, such as nucleotides 54-58 (AAAAA) of the conserved portion of a spyCas9 sgRNA.

Structural alignment involves identifying corresponding residues across two (or more) sequences by (i) modeling the structure of a first sequence using the known structure of the second sequence or (ii) comparing the structures of the first and second sequences where both are known, and identifying the residue in the first sequence most similarly positioned to a residue of interest in the second sequence. Corresponding residues are identified in some algorithms based on distance minimization given position (e.g., nucleobase position 1 or the 1′ carbon of the pentose ring for polynucleotides, or alpha carbons for polypeptides) in the overlaid structures (e.g., what set of paired positions provides a minimized root-mean-square deviation for the alignment). When identifying positions in a non-spyCas9 gRNA corresponding to positions described with respect to spyCas9 gRNA, spyCas9 gRNA can be the “second” sequence. Where a non-spyCas9 gRNA of interest does not have an available known structure, but is more closely related to another non-spyCas9 gRNA that does have a known structure, it may be most effective to model the non-spyCas9 gRNA of interest using the known structure of the closely related non-spyCas9 gRNA, and then compare that model to the spyCas9 gRNA structure to identify the desired corresponding residue in the non-spyCas9 gRNA of interest. There is an extensive literature on structural modeling and alignment for proteins; representative disclosures include U.S. Pat. Nos. 6,859,736; 8,738,343; and those cited in Aslam et al., Electronic Journal of Biotechnology 20 (2016) 9-13. For discussion of modeling a structure based on a known related structure or structures, see, e.g., Bordoli et al., Nature Protocols 4 (2009) 1-13, and references cited therein. See also FIG. 2(F) from Nishimasu et al., Cell 162(5): 1113-1126 (2015) for alignment of nucleic acid.

A “target sequence” as used herein refers to a sequence of nucleic acid to which the guide region directs a nuclease for cleavage. In some embodiments, a spyCas9 protein may be directed by a guide region to a target sequence by the nucleotides present in the guide region. In some embodiments, the sgRNA does not comprise a spacer region.

As used herein, the “5′ end” refers to the first nucleotide of the gRNA (including a dgRNA (typically the 5′ end of the crRNA of the dgRNA), sgRNA or a short-sgRNA), in which the 5′ position is not linked to another nucleotide.

As used herein, a “5′ end modification” refers to a gRNA comprising a guide region having modifications in one or more of the one (1) to about seven (7) nucleotides at its 5′ end, optionally wherein the first nucleotide (from the 5′ end) of the gRNA is modified.

As used herein, the “3′ end” refers to the end or terminal nucleotide of a gRNA, in which the 3′ position is not linked to another nucleotide. In some embodiment, the 3′ end is in the 3′ tail. In some embodiments, the 3′ end is in the conserved portion of an gRNA.

As used herein, a “3′ end modification” refers to a gRNA having modifications in one or more of the one (1) to about seven (7) nucleotides at its 3′ end, optionally wherein the last nucleotide (i.e., the 3′ most nucleotide) of the gRNA is modified. If a 3′ tail is present, the 1 to about 7 nucleotides may be within the 3′ tail. If a 3′ tail is not present, the 1 to about 7 nucleotides may be within the conserved portion of a sgRNA.

The “last,” “second to last,” “third to last,” etc., nucleotide refers to the 3′ most, second 3′ most, third 3′ most, etc., nucleotide, respectively in a given sequence. For example, in the sequence 5′-AAACTG-3′, the last, second to last, and third to last nucleotides are G, T, and C, respectively. The phrase “last 3 nucleotides” refers to the last, second to last, and third to last nucleotides; more generally, “last N nucleotides” refers to the last to the Nth to last nucleotides, inclusive. “Third nucleotide from the 3′ end of the 3′ terminus” is equivalent to “third to last nucleotide.” Similarly, “third nucleotide from the 5′ end of the 5′ terminus” is equivalent to “third nucleotide at the 5′ terminus.”

As used herein, a “protective end modification” (such as a protective 5′ end modification or protective 3′ end modification) refers to a modification of one or more nucleotides within seven nucleotides of the end of an sgRNA that reduces degradation of the sgRNA, such as exonucleolytic degradation. In some embodiments, a protective end modification comprises modifications of at least two or at least three nucleotides within seven nucleotides of the end of the sgRNA. In some embodiments, the modifications comprise phosphorothioate linkages, 2′ modifications such as 2′-OMe or 2′-fluoro, 2′-H (DNA), ENA, UNA, or a combination thereof. In some embodiments, the modifications comprise phosphorothioate linkages and 2′-OMe modifications. In some embodiments, at least three terminal nucleotides are modified, e.g., with phosphorothioate linkages or with a combination of phosphorothioate linkages and 2′-OMe modifications. Modifications known to those of skill in the art to reduce exonucleolytic degradation are encompassed.

In some embodiments, a “3′ tail” comprising between 1 and about 20 nucleotides follows the conserved portion of a sgRNA at its 3′ end.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, also called “Cas protein”, as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity, such as a Cas9 nuclease or a Cpf1 nuclease. Class 2 Cas nucleases include Class 2 Cas cleavases and Class 2 Cas nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. “Cas9” encompasses Spy Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is RNA or modified RNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of a nucleic acid phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

As used herein, the “minimum uridine content” of a given ORF is the uridine content of an ORF that (a) uses a minimal uridine codon at every position and (b) encodes the same amino acid sequence as the given ORF. The minimal uridine codon(s) for a given amino acid is the codon(s) with the fewest uridines (usually 0 or 1 except for a codon for phenylalanine, where the minimal uridine codon has 2 uridines). Modified uridine residues are considered equivalent to uridines for the purpose of evaluating minimum uridine content.

As used herein, the “minimum uridine dinucleotide content” of a given ORF is the lowest possible uridine dinucleotide (UU) content of an ORF that (a) uses a minimal uridine codon (as discussed above) at every position and (b) encodes the same amino acid sequence as the given ORF. The uridine dinucleotide (UU) content can be expressed in absolute terms as the enumeration of UU dinucleotides in an ORF or on a rate basis as the percentage of positions occupied by the uridines of uridine dinucleotides (for example, AUUAU would have a uridine dinucleotide content of 40% because 2 of 5 positions are occupied by the uridines of a uridine dinucleotide). Modified uridine residues are considered equivalent to uridines for the purpose of evaluating minimum uridine dinucleotide content.

As used herein, the “minimum adenine content” of a given open reading frame (ORF) is the adenine content of an ORF that (a) uses a minimal adenine codon at every position and (b) encodes the same amino acid sequence as the given ORF. The minimal adenine codon(s) for a given amino acid is the codon(s) with the fewest adenines (usually 0 or 1 except for a codon for lysine and asparagine, where the minimal adenine codon has 2 adenines). Modified adenine residues are considered equivalent to adenines for the purpose of evaluating minimum adenine content.

As used herein, the “minimum adenine dinucleotide content” of a given open reading frame (ORF) is the lowest possible adenine dinucleotide (AA) content of an ORF that (a) uses a minimal adenine codon (as discussed above) at every position and (b) encodes the same amino acid sequence as the given ORF. The adenine dinucleotide (AA) content can be expressed in absolute terms as the enumeration of AA dinucleotides in an ORF or on a rate basis as the percentage of positions occupied by the adenines of adenine dinucleotides (for example, UAAUA would have an adenine dinucleotide content of 40% because 2 of 5 positions are occupied by the adenines of an adenine dinucleotide). Modified adenine residues are considered equivalent to adenines for the purpose of evaluating minimum adenine dinucleotide content.

As used herein, a “subject” refers to any member of the animal kingdom. In some embodiments, “subject” refers to humans. In some embodiments, “subject” refers to non-human animals. In some embodiments, “subject” refers to primates. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically-engineered animal, and/or a clone. In certain embodiments of the present invention the subject is an adult, an adolescent or an infant. In some embodiments, terms “individual” or “patient” are used and are intended to be interchangeable with “subject”.

Types of Modifications described herein

Guide RNAs (e.g., sgRNAs, short-sgRNAs, dgRNAs, and crRNAs) comprising modifications at various positions are disclosed herein. In some embodiments, a position of a gRNA that comprises a modification is modified with any one or more of the following types of modifications.

### 2′-O-methyl Modifications

Modified sugars are believed to control the puckering of nucleotide sugar rings, a physical property that influences oligonucleotide binding affinity for complementary strands, duplex formation, and interaction with nucleases. Substitutions on sugar rings can therefore alter the conformation and puckering of these sugars. For example, 2′-O-methyl (2′-OMe) modifications can increase binding affinity and nuclease stability of oligonucleotides, though as shown in the Examples, the effect of any modification at a given position in an oligonucleotide needs to be empirically determined.

The terms “mA,” “mC,” “mU,” or “mG” may be used to denote a nucleotide that has been modified with 2′-OMe.

A ribonucleotide and a modified 2′-O-methyl ribonucleotide can be depicted as follows:

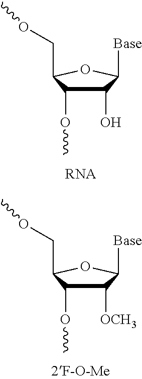

2′-O-(2-methoxyethyl) Modifications

In some embodiments, the modification may be 2′-O-(2-methoxyethyl) (2′-O-moe). A modified 2′-O-moe ribonucleotide can be depicted as follows:

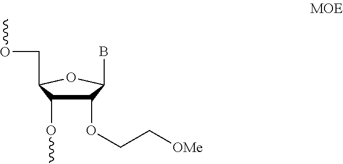

The terms “moeA,” “moeC,” “moeU,” or “moeG” may be used to denote a nucleotide that has been modified with 2′-O-moe.

2′-fluoro Modifications

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” may be used to denote a nucleotide that has been substituted with 2′-F.

A ribonucleotide without and with a 2′-F substitution can be depicted as follows:

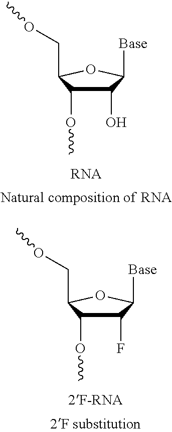

### Phosphorothioate Modifications

A phosphorothioate (PS) linkage or bond refers to a bond where a sulfur is substituted for one nonbridging phosphate oxygen in a phosphodiester linkage, for example between nucleotides. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond. Throughout this application, PS modifications are grouped with the nucleotide whose 3′ carbon is bonded to the phosphorothioate; thus, indicating that a PS modification is at position 1 means that the phosphorothioate is bonded to the 3′ carbon of nucleotide 1 and the 5′ carbon of nucleotide 2. Thus, where a YA site is indicated as being “PS modified” or the like, the PS linkage is between the Y and A or between the A and the next nucleotide.

In this application, the terms “mA*,” “mC*,” “mU*,” or “mG*” may be used to denote a nucleotide that has been substituted with 2′-OMe and that is linked to the next (e.g., 3′) nucleotide with a PS linkage, which may sometimes be referred to as a “PS bond.” Similarly, the terms “fA*,” “fC*,” “fU*,” or “fG*” may be used to denote a nucleotide that has been substituted with 2′-F and that is linked to the next (e.g., 3′) nucleotide with a PS linkage. Equivalents of a PS linkage or bond are encompassed by embodiments described herein.

The diagram below shows the substitution of S- for a nonbridging phosphate oxygen, generating a PS linkage in lieu of a phosphodiester linkage:

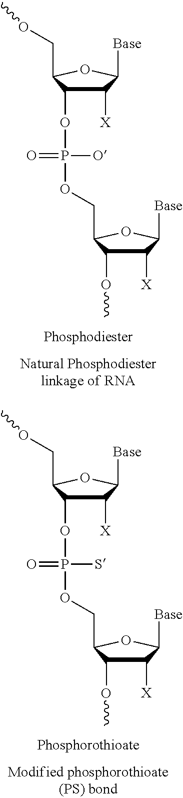

### Inverted Abasic Modifications

Abasic nucleotides refer to those which lack nitrogenous bases. The figure below depicts an oligonucleotide with an abasic (in this case, shown as apurinic; an abasic site could also be an apyrimidinic site, wherein the description of the abasic site is typically in reference to Watson-Crick base pairing—e.g., an apurinic site refers to a site that lacks a nitrogenous base and would typically base pair with a pyrimidinic site) site that lacks a base, wherein the base may be substituted by another moiety at the 1′ position of the furan ring (e.g., a hydroxyl group, as shown below, to form a ribose or deoxyribose site, as shown below, or a hydrogen):

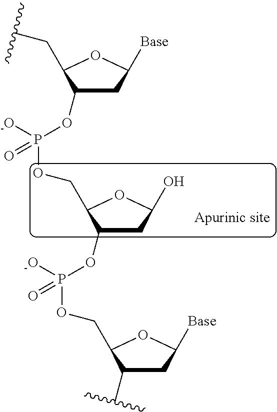

Inverted bases refer to those with linkages that are inverted from the normal 5′ to 3′ linkage (i.e., either a 5′ to 5′ linkage or a 3′ to 3′ linkage). For example:

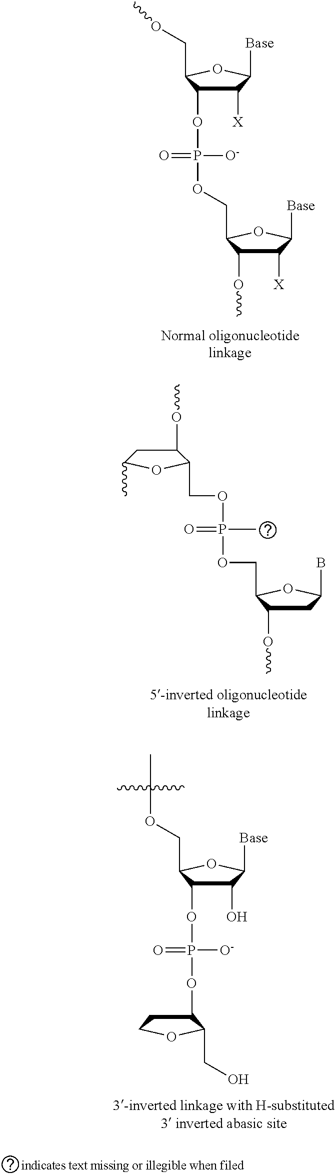

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap. In this application, the terms “invd” indicates an inverted abasic nucleotide linkage.

### Deoxyribonucleotides

A deoxyribonucleotide (in which the sugar comprises a 2′-deoxy position) is considered a modification in the context of a gRNA, in that the nucleotide is modified relative to standard RNA by the substitution of a proton for a hydroxyl at the 2′ position. Unless otherwise indicated, a deoxyribonucleotide modification at a position that is U in an unmodified RNA can also comprise replacement of the U nucleobase with a T.

### Bicyclic Ribose Analog

Exemplary bicyclic ribose analogs include locked nucleic acid (LNA), ENA, bridged nucleic acid (BNA), or another LNA-like modifications. In some instances, a bicyclic ribose analog has 2′ and 4′ positions connected through a linker. The linker can be of the formula —X—(CH₂)ₙ— where n is 1 or 2; X is O, NR, or S; and R is H or C₁₋₃ alkyl, e.g., methyl. Examples of bicyclic ribose analogs include LNAs comprising a 2′-O—CH₂-4′ bicyclic structure (oxy-LNA) (see WO 98/39352 and WO 99/14226); 2′-NH—CH₂-4′ or 2′-N(CH₃)—CH₂-4′ (amino-LNAs) (Singh et al., J. Org. Chem. 63:10035-10039 (1998); Singh et al., J. Org. Chem. 63:6078-6079 (1998)); and 2′-S—CH₂-4′ (thio-LNA) (Singh et al., J. Org. Chem. 63:6078-6079 (1998); Kumar et al., Biorg. Med. Chem. Lett. 8:2219-2222 (1998)).

### ENA

An ENA modification refers to a nucleotide comprising a 2′-O,4′-C-ethylene modification. An exemplary structure of an ENA nucleotide is shown below, in which wavy lines indicate connections to the adjacent nucleotides (or terminal positions as the case may be, with the understanding that if the 3′ terminal nucleotide is an ENA nucleotide, the 3′ position may comprise a hydroxyl rather than phosphate). For further discussion of ENA nucleotides, see, e.g., Koizumi et al., Nucleic Acids Res. 31: 3267-3273 (2003).

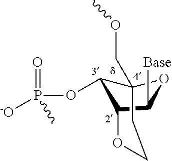

### UNA

A UNA or unlocked nucleic acid modification refers to a nucleotide comprising a 2′,3′-seco-RNA modification, in which the 2′ and 3′ carbons are not bonded directly to each other. An exemplary structure of a UNA nucleotide is shown below, in which wavy lines indicate connections to the adjacent phosphates or modifications replacing phosphates (or terminal positions as the case may be). For further discussion of UNA nucleotides, see, e.g., Snead et al., Molecular Therapy 2: e103, doi:10.1038/mtna.2013.36 (2013).

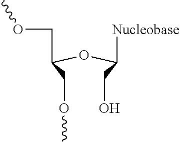

### Base Modifications

A base modification is any modification that alters the structure of a nucleobase or its bond to the backbone, including isomerization (as in pseudouridine). In some embodiments, a base modification includes inosine. In some embodiments, a modification comprises a base modification that reduces RNA endonuclease activity, e.g., by interfering with recognition of a cleavage site by an RNase and/or by stabilizing an RNA structure (e.g., secondary structure) that decreases accessibility of a cleavage site to an RNase. Exemplary base modifications that can stabilize RNA structures are pseudouridine and 5-methylcytosine. See Peacock et al., J Org Chem. 76: 7295-7300 (2011). In some embodiments, a base modification can increase or decrease the melting temperature (Tm) of a nucleic acid, e.g., by increasing the hydrogen bonding in a Watson-Crick base pair, forming non-canonical base pair, or creating a mismatched base pair.

The above modifications and their equivalents are included within the scope of the embodiments described herein.

### YA Modifications

A modification at a YA site (also referred to as a YA modification) can be a modification of the internucleoside linkage, a modification of the base (pyrimidine or adenine), e.g. by chemical modification, substitution, or otherwise, and/or a modification of the sugar (e.g. at the 2′ position, such as 2′-O-alkyl, 2′-F, 2′-moe, 2′-F arabinose, 2′-H (deoxyribose), and the like). In some embodiments, a “YA modification” is any modification that alters the structure of the dinucleotide motif to reduce RNA endonuclease activity, e.g., by interfering with recognition or cleavage of a YA site by an RNase and/or by stabilizing an RNA structure (e.g., secondary structure) that decreases accessibility of a cleavage site to an RNase. See Peacock et al., J Org Chem. 76: 7295-7300 (2011); Behlke, Oligonucleotides 18:305-320 (2008); Ku et al., Adv. Drug Delivery Reviews 104: 16-28 (2016); Ghidini et al., Chem. Commun., 2013, 49, 9036. Peacock et al., Belhke, Ku, and Ghidini provide exemplary modifications suitable as YA modifications. Modifications known to those of skill in the art to reduce endonucleolytic degradation are encompassed. Exemplary 2′ ribose modifications that affect the 2′ hydroxyl group involved in RNase cleavage are 2′-H and 2′-O-alkyl, including 2′-O-Me. Modifications such as bicyclic ribose analogs, UNA, and modified internucleoside linkages of the residues at the YA site can be YA modifications. Exemplary base modifications that can stabilize RNA structures are pseudouridine and 5-methylcytosine. In some embodiments, at least one nucleotide of the YA site is modified. In some embodiments, the pyrimidine (also called “pyrimidine position”) of the YA site comprises a modification (which includes a modification altering the internucleoside linkage immediately 3′ of the sugar of the pyrimidine, a modification of the pyrimidine base, and a modification of the ribose, e.g. at its 2′ position). In some embodiments, the adenine (also called “adenine position”) of the YA site comprises a modification (which includes a modification altering the internucleoside linkage immediately 3′ of the sugar of the pyrimidine, a modification of the pyrimidine base, and a modification of the ribose, e.g. at its 2′ position). In some embodiments, the pyrimidine and the adenine of the YA site comprise modifications. In some embodiments, the YA modification reduces RNA endonuclease activity.

The above modifications and their equivalents are included within the scope of the embodiments described herein.

Domains/regions of sgRNAs

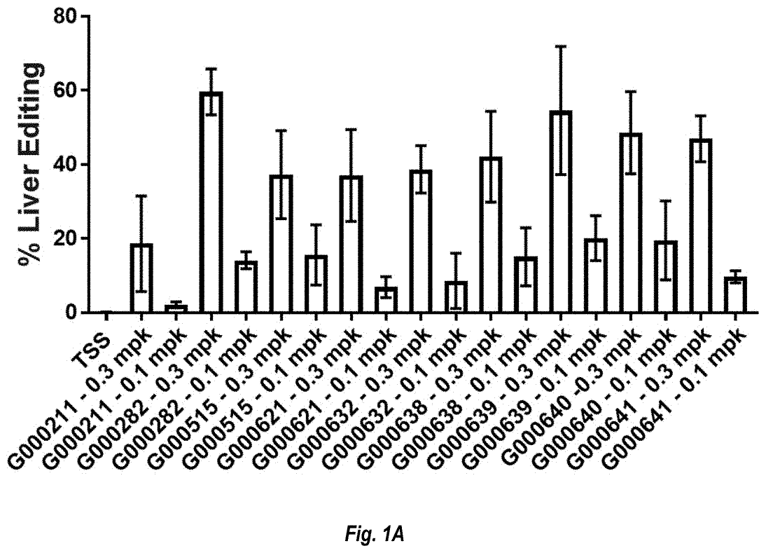

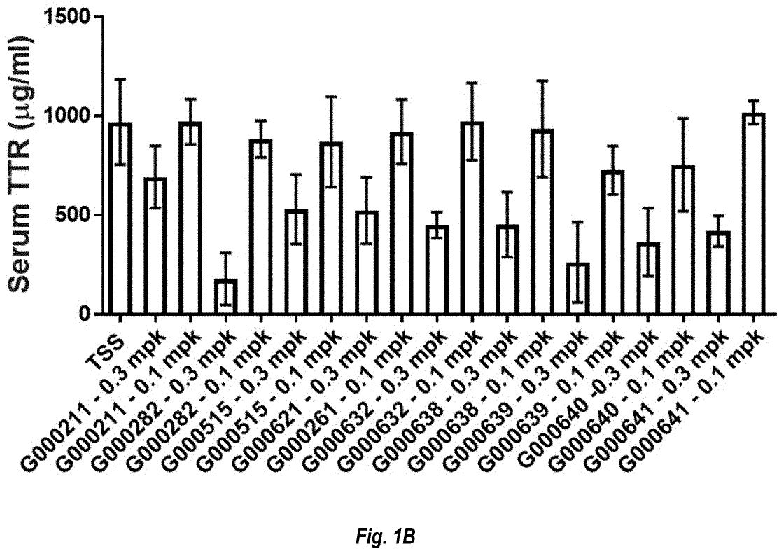

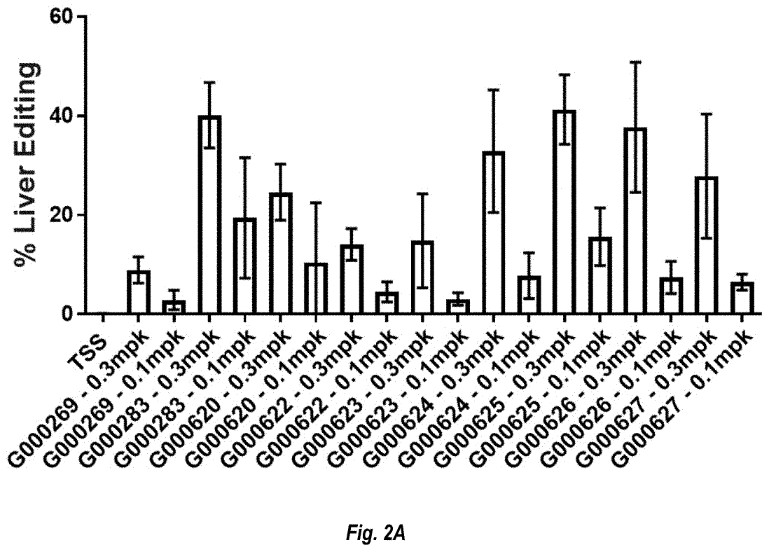

Briner A E et al., Molecular Cell 56:333-339 (2014) describes functional domains of sgRNAs, referred to herein as “domains”, including the “spacer” domain responsible for targeting, the “lower stem”, the “bulge”, “upper stem” (which may include a tetraloop), the “nexus”, and the “hairpin 1” and “hairpin 2” domains. See, Briner et al. at page 334, FIG. 1A.

Table 3 provides a schematic of the domains of an sgRNA as used herein. In Table 3, the “n” between regions represents a variable number of nucleotides, for example, from 0 to 1, 2, 3, 4, 5, 6, 7, 8, 9, 10, 11, 12, 13, 14, 15, 16, 17, 18, 19, 20, or more. In some embodiments, n equals 0. In some embodiments, n equals 1.

5′ Terminus Region

In some embodiments, the sgRNA or short-sgRNA comprises nucleotides at the 5′ terminus as shown in Table 3. In some embodiments, the 5′ terminus of the sgRNA or short-sgRNA comprises a spacer or guide region that functions to direct a Cas protein, e.g., a Cas9 protein, to a target nucleotide sequence. In some embodiments, the 5′ terminus does not comprise a guide region. In some embodiments, the 5′ terminus comprises a spacer and additional nucleotides that do not function to direct a Cas protein to a target nucleotide region.

Lower Stem

In some embodiments, the sgRNA or short-sgRNA comprises a lower stem (LS) region that when viewed linearly, is separated by a bulge and upper stem regions. See Table 3.

In some embodiments, the lower stem regions comprise 1-12 nucleotides, e.g. in one embodiment the lower stem regions comprise LS1-LS12. In some embodiments, the lower stem region comprises fewer nucleotides than shown in Table 3. In some embodiments, the lower stem region comprises more nucleotides than shown in Table 3. When the lower stem region comprises fewer or more nucleotides than shown in the schematic of Table 3, the modification pattern, as will be apparent to the skilled artisan, should be maintained.

In some embodiments, the lower stem region has nucleotides that are complementary in nucleic acid sequence when read in opposite directions. In some embodiments, the complementarity in nucleic acid sequence of lower stem leads to a secondary structure of a stem in the sgRNA or short-sgRNA (e.g., the regions may base pair with one another). In some embodiments, the lower stem regions may not be perfectly complimentary to each other when read in opposite directions.

Bulge

In some embodiments, the sgRNA or short-sgRNA comprises a bulge region comprising six nucleotides, B1-B6. When viewed linearly, the bulge region is separated into two regions. See Table 3. In some embodiments, the bulge region comprises six nucleotides, wherein the first two nucleotides are followed by an upper stem region, followed by the last four nucleotides of the bulge. In some embodiments, the bulge region comprises fewer nucleotides than shown in Table 3. In some embodiments, the bulge region comprises more nucleotides than shown in Table 3. When the bulge region comprises fewer or more nucleotides than shown in the schematic of Table 3, the modification pattern, as will be apparent to the skilled artisan, should be maintained.

In some embodiments, the presence of a bulge results in a directional kink between the upper and lower stem modules in an sgRNA or short-sgRNA.

Upper Stem

In some embodiments, the sgRNA or short-sgRNA comprises an upper stem region comprising 12 nucleotides. In some embodiments, the upper stem region comprises a loop sequence. In some instances, the loop is a tetraloop (loop consisting of four nucleotides).

In some embodiments, the upper stem region comprises fewer nucleotides than shown in Table 3. In some embodiments, the upper stem region comprises more nucleotides than shown in Table 3. When the upper stem region comprises fewer or more nucleotides than shown in the schematic of Table 3, the modification pattern, as will be apparent to the skilled artisan, should be maintained.

In some embodiments, the upper stem region has nucleotides that are complementary in nucleic acid sequence when read in opposite directions. In some embodiments, the complementarity in nucleic acid sequence of upper stem leads to a secondary structure of a stem in the sgRNA or short-sgRNA (e.g., the regions may base pair with one another). In some embodiments, the upper stem regions may not be perfectly complimentary to each other when read in opposite directions.

Nexus

In some embodiments, the sgRNA or short-sgRNA comprises a nexus region that is located between the lower stem region and the hairpin 1 region. In some embodiments, the nexus comprises 18 nucleotides. In some embodiments, the nexus region comprises nucleotides N1 through N18 as shown in Table 3.

In some embodiments, the nexus region comprises fewer nucleotides than shown in Table 3. In some embodiments, the nexus region comprises more nucleotides than shown in Table 3. When the nexus region comprises fewer or more nucleotides than shown in the schematic of Table 3, the modification pattern, as will be apparent to the skilled artisan, should be maintained.

In some embodiments, the nexus region has nucleotides that are complementary in nucleic acid sequence when read in opposite directions. In some embodiments, the complementarity in nucleic acid sequence leads to a secondary structure of a stem and/or stem loop in the sgRNA or short-sgRNA (e.g., certain nucleotides in the nexus region may base pair with one another). In some embodiments, the nexus regions may not be perfectly complimentary to each other when read in opposite directions.

Hairpin

In some embodiments, the sgRNA or short-sgRNA comprises one or more hairpin regions. In some embodiments, the hairpin region is downstream of (e.g., 3′ to) the nexus region. In some embodiments, the region of nucleotides immediately downstream of the nexus region is termed “hairpin 1” or “H1”. In some embodiments, the region of nucleotides 3′ to hairpin 1 is termed “hairpin 2” or “H2”. In some embodiments, the hairpin region comprises both hairpin 1 and hairpin 2. In some embodiments, the sgRNA or short-sgRNA comprises hairpin 1 or hairpin 2.

In some embodiments, the hairpin 1 region comprises 12 nucleic acids immediately downstream of the nexus region. In some embodiments, the hairpin 1 region comprises nucleotides H1-1 through H1-12 as shown in Table 3.

In some embodiments, the hairpin 2 region comprises 15 nucleic acids downstream of the hairpin 1 region. In some embodiments, the hairpin 2 region comprises nucleotides H2-1 through H2-15 as shown in Table 3.

In some embodiments, one or more nucleotides is present between the hairpin 1 and the hairpin 2 regions. The one or more nucleotides between the hairpin 1 and hairpin 2 region may be modified or unmodified. In some embodiments, hairpin 1 and hairpin 2 are separated by one nucleotide. In some embodiments, the hairpin regions comprise fewer nucleotides than shown in Table 3. In some embodiments, the hairpin regions comprise more nucleotides than shown in Table 3. When a hairpin region comprises fewer or more nucleotides than shown in the schematic of Table 3, the modification pattern, as will be apparent to the skilled artisan, should be maintained.

In some embodiments, a hairpin region has nucleotides that are complementary in nucleic acid sequence when read in opposite directions. In some embodiments, the hairpin regions may not be perfectly complimentary to each other when read in opposite directions (e.g., the top or loop of the hairpin comprises unpaired nucleotides).

In some embodiments, the sgRNA or short-sgRNA comprises replacement of hairpin 1 with nucleotides “n”, wherein “n” is an integer between 1 and 50, 40, 30, 20, 15, 10, 5, 4, 3, and 2. In some embodiments, the hairpin 1 region of an sgRNA is replaced by 2 nucleotides.

3′ Terminus

The sgRNA or short-sgRNA has a 3′ end, which is the last nucleotide of the sgRNA. The 3′ terminus region includes the last 1-7 nucleotides from the 3′ end. In some embodiments, the 3′ end is the end of hairpin 2. In some embodiments, the sgRNA comprises nucleotides after the hairpin region(s). In some embodiments, the sgRNA includes a 3′ tail region, in which case the last nucleotide of the 3′ tail is the 3′ terminus. In some embodiments, the 3′ tail comprises 1, 2, 3, 4, 5, 6, 7, 8, 9, 10, 15 or 20 or more nucleotides, e.g. that are not associated with the secondary structure of a hairpin. In some embodiments, the 3′ tail region comprises 1, 2, 3, or 4 nucleotides that are not associated with the secondary structure of a hairpin. In some embodiments, the 3′ tail region comprises 4 nucleotides that are not associated with the secondary structure of a hairpin. In some embodiments, the 3′ tail region comprises 1, 2, or 3 nucleotides that are not associated with the secondary structure of a hairpin.

TABLE 2
(Conserved Portion of a spyCas9 sgRNA; SEQ ID NO: 400)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 |
|---|---|---|---|---|---|---|---|---|---|---|---|---|---|---|---|---|---|---|---|---|---|---|---|
| G | U | U | U | U | A | G | A | G | C | U | A | G | A | A | A | U | A | G | C | A | A | G | U |

| | | | |
|---|---|---|---|
| LS1-LS6 | B1-B2 | US1-US12 | B2-B6 |

| 25 | 26 | 27 | 28 | 29 | 30 | 31 | 32 | 33 | 34 | 35 | 36 | 37 | 38 | 39 | 40 | 41 | 42 | 43 | 44 | 45 | 46 | 47 | 48 |
|---|---|---|---|---|---|---|---|---|---|---|---|---|---|---|---|---|---|---|---|---|---|---|---|
| U | A | A | A | A | U | A | A | G | G | C | U | A | G | U | C | C | G | U | U | A | U | C | A |

| | | | | | | Nexus | | | | | | | | | | | | | | | | | | | | | |
|---|---|---|---|---|---|---|---|---|---|---|---|---|---|---|---|---|---|---|---|---|---|---|---|---|---|---|---|
| 49 | 50 | 51 | 52 | 53 | 54 | 55 | 56 | 57 | 58 | 59 | 60 | 61 | 62 | 63 | 64 | 65 | 66 | 67 | 68 | 69 | 70 | 71 | 72 | 73 | 74 | 75 | 76 |
| A | C | U | U | G | A | A | A | A | A | G | U | G | G | C | A | C | C | G | A | G | U | C | G | G | U | G | C |

| | | |
|---|---|---|
| H1-1 through H1-12 | N | H2-1 through H2-15 |

TABLE 3
(Regions of sgRNA (linear view, 5′ to 3′)

| | LS1-6 | | B1-2 | | US1-12 | | B3-6 | |
|---|---|---|---|---|---|---|---|---|
| 5′ terminus (n) | lower stem | n | bulge | n | upper stem | n | bulge | n |

| LS7-12 | | N1-18 | | H1-1 thru H1-12 | | H2-1 thru H2-15 | |
|---|---|---|---|---|---|---|---|
| lower stem | n | nexus | n | hairpin 1 | n | hairpin 2 | 3′ terminus |

gRNAs comprising Modifications, including Modifications of YA Sites

In some embodiments, a gRNA (e.g., sgRNA, short-sgRNA, dgRNA, or crRNA) described herein comprises modifications at 1, 2, 3, 4, 5, 6, 7, 8, 9, 10, 11, 12, 13, 14, 15, 16, or more YA sites (e.g., in the conserved region and/or the guide region) and/or a modification, such as a YA modification, at one or more nucleotides located at or after nucleotide 6 from the 5′ end of the 5′ terminus. In some embodiments, the pyrimidine of the YA site comprises a modification (which includes a modification altering the internucleoside linkage immediately 3′ of the sugar of the pyrimidine). In some embodiments, the adenine of the YA site comprises a modification (which includes a modification altering the internucleoside linkage immediately 3′ of the sugar of the adenine). In some embodiments, the pyrimidine and the adenine of the YA site comprise modifications, such as sugar, base, or internucleoside linkage modifications. The YA modifications can be any of the types of modifications set forth herein. In some embodiments, the YA modifications comprise one or more of phosphorothioate, 2′-OMe, or 2′-fluoro. In some embodiments, the YA modifications comprise pyrimidine modifications comprising one or more of phosphorothioate, 2′-OMe, or 2′-fluoro. In some embodiments, the YA modification comprises a bicyclic ribose analog (e.g., an LNA, BNA, or ENA) within an RNA duplex region that contains one or more YA sites. In some embodiments, the YA modification comprises a bicyclic ribose analog (e.g., an LNA, BNA, or ENA) within an RNA duplex region that contains a YA site, wherein the YA modification is distal to the YA site.

Any of the embodiments described above may be combined with the following: (i) at least one of nucleotides 8-11, 13, 14, 17, or 18 from the 5′ end of the 5′ terminus does not comprise a 2′-fluoro modification, and/or (ii) at least one of nucleotides 6-10 from the 5′ end of the 5′ terminus does not comprise a phosphorothioate linkage; and (i) at least one of nucleotides 7-10 from the 5′ end of the 5′ terminus does not comprise a 2′-OMe modification, (ii) nucleotide 20 from the 5′ end of the 5′ terminus does not comprise a 2′-OMe modification, and/or (iii) or the guide RNA comprises a 2′-fluoro modification at any one or more of nucleotides 1-20 from the 5′ end of the 5′ terminus and at least one of nucleotides 11, 12, 13, 14, 17, or 18 from the 5′ end of the 5′ terminus does not comprise a 2′-fluoro modification, optionally wherein nucleotide 12 from the 5′ end of the 5′ terminus does not comprise a 2′-fluoro modification. Such embodiments may be further, or alternatively, combined with any other one or more embodiments described herein to the extent feasible.

Guide Region Modifications including YA Site Modifications

In some embodiments, the guide region comprises one or more modifications, optionally including YA site modifications. In some embodiments, the guide region comprises 1, 2, 3, 4, 5, or more YA sites (“guide region YA sites”) that may comprise YA modifications. In some embodiments, one or more YA sites located at 5-end, 6-end, 7-end, 8-end, 9-end, or 10-end from the 5′ end of the 5′ terminus (where “5-end”, etc., refers to position 5 to the 3′ end of the guide region, i.e., the most 3′ nucleotide in the guide region) comprise YA modifications. In some embodiments, two or more YA sites located at 5-end, 6-end, 7-end, 8-end, 9-end, or 10-end from the 5′ end of the 5′ terminus comprise YA modifications. In some embodiments, three or more YA sites located at 5-end, 6-end, 7-end, 8-end, 9-end, or 10-end from the 5′ end of the 5′ terminus comprise YA modifications. In some embodiments, four or more YA sites located at 5-end, 6-end, 7-end, 8-end, 9-end, or 10-end from the 5′ end of the 5′ terminus comprise YA modifications. In some embodiments, five or more YA sites located at 5-end, 6-end, 7-end, 8-end, 9-end, or 10-end from the 5′ end of the 5′ terminus comprise YA modifications. A modified guide region YA site comprises a YA modification.

In some embodiments, a modified guide region YA site is within 17, 16, 15, 14, 13, 12, 11, 10, or 9 nucleotides of the 3′ terminal nucleotide of the guide region. For example, if a modified guide region YA site is within 10 nucleotides of the 3′ terminal nucleotide of the guide region and the guide region is 20 nucleotides long, then the modified nucleotide of the modified guide region YA site is located at any of positions 11-20. In some embodiments, a YA modification is located within a YA site 20, 19, 18, 17, 16, 15, 14, 13, 12, 11, 10, 9, 8, 7, 6, 5, 4, 3, 2, or 1 nucleotides from the 3′ terminal nucleotide of the guide region. In some embodiments, a YA modification is located 20, 19, 18, 17, 16, 15, 14, 13, 12, 11, 10, 9, 8, 7, 6, 5, 4, 3, 2, or 1 nucleotides from the 3′ terminal nucleotide of the guide region.

In some embodiments, a modified guide region YA site is at or after nucleotide 4, 5, 6, 7, 8, 9, 10, or 11 from the 5′ end of the 5′ terminus.

In some embodiments, a modified guide region YA site is other than a 5′ end modification. For example, a gRNA can comprise a 5′ end modification as described herein and further comprise a modified guide region YA site. Alternatively, a gRNA can comprise an unmodified 5′ end and a modified guide region YA site. Alternatively, a gRNA can comprise a modified 5′ end and an unmodified guide region YA site.

In some embodiments, a modified guide region YA site comprises a modification that at least one nucleotide located 5′ of the guide region YA site does not comprise. For example, if nucleotides 1-3 comprise phosphorothioates, nucleotide 4 comprises only a 2′-OMe modification, and nucleotide 5 is the pyrimidine of a YA site and comprises a phosphorothioate, then the modified guide region YA site comprises a modification (phosphorothioate) that at least one nucleotide located 5′ of the guide region YA site (nucleotide 4) does not comprise. In another example, if nucleotides 1-3 comprise phosphorothioates, and nucleotide 4 is the pyrimidine of a YA site and comprises a 2′-OMe, then the modified guide region YA site comprises a modification (2′-OMe) that at least one nucleotide located 5′ of the guide region YA site (any of nucleotides 1-3) does not comprise. This condition is also always satisfied if an unmodified nucleotide is located 5′ of the modified guide region YA site.

In some embodiments, the guide region comprises modifications at 1-14 of nucleotides 1, 2, 3, 4, 6, 7, 8, 9, 10, 11, 13, 14, 17, and 18 of the guide region. Such modifications may be 2′-OMe, 2′-fluoro, 2′-H, inosine, or phosphorothioate modifications, or a combination thereof. For example, 2′-OMe modifications may be included at any or all of nucleotides 1-4 and 12; phosphorothioate modifications may be included at any or all of nucleotides 1-3 and 6-10; and/or 2′-fluoro modifications may be included at any or all of nucleotides 8-11, 13, 14, 17, and 18. In negative terms, 2′-OMe modifications may be excluded from nucleotides 6-11 and 13-end; 2′-fluoro modifications may be excluded from nucleotides 1-7, 15, 16, and 20 (if present); and/or phosphorothioate modifications may be excluded from nucleotides 4, 5, 11-14, 17, and 18. In some embodiments, nucleotides are modified in a YA-site dependent manner, e.g., if a YA site is present at any of nucleotides 5-6, 12-13, 15-16, 16-17, or 19-20, then at least one nucleotide of the YA site is modified, e.g., at least the pyrimidine of the YA site is modified, optionally wherein the nucleotides at positions 5, 12, 15, 16, and 19 are unmodified if they are not the pyrimidine of a YA site. In some embodiments, the modification at nucleotide 5 when it is the pyrimidine of a YA site is 2′-OMe; the modification at nucleotide 12 when it is the pyrimidine of a YA site is 2′-OMe; the modification at nucleotide 15 when it is the pyrimidine of a YA site is phosphorothioate; the modification at nucleotide 16 when it is the pyrimidine of a YA site is phosphorothioate; and/or the modification at nucleotide 19 when it is the pyrimidine of a YA site is phosphorothioate. Recognizing that YA sites cannot be present at both positions 15-16 and 16-17, it is thus possible for there to be up to four modifications contingent on the presence of YA sites. In an alternative embodiment, the modification at nucleotide 19 may instead be a 2′-fluoro. This can be present in a YA site-dependent manner or it can be present regardless of whether there is a YA site at positions 19-20. In some embodiments, nucleotide 15 and 16 are unmodified or modified only with a phosphorothioate, e.g., only at a nucleotide which is the pyrimidine of a YA site located at nucleotides 15-16 or 16-17. In some embodiments, nucleotides 15 and 16 comprise unmodified riboses and/or unmodified nucleobases. In some embodiments, nucleotide 5 is unmodified or is modified only with 2′-OMe if it is the pyrimidine of a YA site. In some embodiments, nucleotide 12 is unmodified or is modified only with 2′-OMe if it is the pyrimidine of a YA site. In some embodiments, nucleotide 20 (or the 3′-terminal nucleotide of the guide region) is unmodified. In any of the foregoing embodiments, the guide region may consist of 20 nucleotides.

In some embodiments, a gRNA comprises a guide region that comprises a modification at one or more of nucleotide 5 and/or 12. The modifications at nucleotide 5 and/or 12 may be independently selected from modifications described herein, e.g., 2′-OMe, 2′-F, phosphorothioate, and 2′-H (a deoxyribonucleotide). Such modifications may be combined with another modification pattern or nucleotide modifications described herein, e.g., as shown in a gRNA described herein. Particular examples of such embodiments are described herein, e.g., in certain numbered embodiments set forth above and in modification patterns represented by sequences in the Table of Sequences. In some embodiments, such a modification is combined with one or more, or all, of 2′-OMe modifications at nucleotides 1-4, phosphorothioate modifications at nucleotides 1-3 and 6-10, and/or 2′-F modifications at nucleotides 8-11, 13, 14, 17, and 18.

In some embodiments, a gRNA comprises a guide region that comprises modifications at any one, two, or all of nucleotides 8-10. Such modifications may be combined with another modification pattern or nucleotide modifications described herein, e.g., as shown in a gRNA described herein. The modifications may be independently selected from modifications described herein, e.g., 2′-F modifications and phosphorothioate modifications, or a combination thereof. In some embodiments, any one, two, or all of nucleotides 8-10 comprise 2′-F modifications. In some embodiments, any one, two, or all of nucleotides 8-10 comprise 2′-F modifications but not phosphorothioate modifications. In some embodiments, any one, two, or all of nucleotides 8-10 comprise 2′-F modifications and phosphorothioate modifications. Particular examples of such embodiments are described herein, e.g., in certain numbered embodiments set forth above and in modification patterns represented by sequences in the Table of Sequences. In some embodiments, such a modification is combined with one or more, or all, of 2′-OMe modifications at nucleotides 1-4, phosphorothioate modifications at nucleotides 1-3 and 6-7, and/or 2′-F modifications at nucleotides 11, 13, 14, 17, and 18.

In some embodiments, a gRNA comprises a guide region that comprises modifications at any one or both of nucleotides 5 and 6. The modifications may be independently selected from modifications described herein, e.g., 2′-F modifications and phosphorothioate modifications, or a combination thereof. In some embodiments, any one or both of nucleotides 5 and 6 comprise 2′-F modifications. In some embodiments, any one or both of nucleotides 5 and 6 comprise 2′-F modifications but not phosphorothioate modifications. In some embodiments, any one or both of nucleotides 5 and 6 comprise 2′-F modifications and phosphorothioate modifications. Particular examples of such embodiments are described herein, e.g., in certain numbered embodiments set forth above and in modification patterns represented by sequences in the Table of Sequences. In some embodiments, such a modification is combined with one or more, or all, of 2′-OMe modifications at nucleotides 1-4, phosphorothioate modifications at nucleotides 1-3 and 7-10, and/or 2′-F modifications at nucleotides 8-11, 13, 14, 17, and 18.

In some embodiments, a gRNA comprises a guide region that comprises modifications at at least 1, 2, 3, 4, 5, or 6 of nucleotides 6-11. The modifications may be independently selected from modifications described herein, e.g., 2′-F modifications. In some embodiments, 2′-F modifications at 1, 2, 3, 4, 5, or 6 of nucleotides 6-11 are combined with another compatible modification, such as a phosphorothioate modification, at one or more of the positions comprising a 2′-F modification. Particular examples of such embodiments are described herein, e.g., in certain numbered embodiments set forth above and in modification patterns represented by sequences in the Table of Sequences. In some embodiments, such a modification is combined with one or more, or all, of 2′-OMe modifications at nucleotides 1-4, phosphorothioate modifications at nucleotides 1-3, and/or 2′-F modifications at nucleotides 13, 14, 17, and 18.

In some embodiments, a gRNA comprises a guide region that comprises modifications at at least 1, 2, 3, 4, 5, 6, 7, 8, 9, or 10 of nucleotides 1-4 and 6-11. The modifications may be independently selected from modifications described herein, e.g., 2′-F modifications. In some embodiments, 2′-F modifications at 1, 2, 3, 4, 5, 6, 7, 8, 9, or 10 of nucleotides 1-4 and 6-11 are combined with another compatible modification, such as a phosphorothioate modification, at one or more of the positions comprising a 2′-F modification. Particular examples of such embodiments are described herein, e.g., in certain numbered embodiments set forth above and in modification patterns represented by sequences in the Table of Sequences. In some embodiments, such a modification is combined with one or more, or all, of phosphorothioate modifications at nucleotides 1-3 and/or 2′-F modifications at nucleotides 13, 14, 17, and 18.

In some embodiments, a gRNA comprises a guide region that comprises 2′-OMe modifications at at least 1, 2, 3, or 4 of nucleotides 9, 11, 13, and 14. In some embodiments, 2′-OMe modifications at at least 1, 2, 3, or 4 of nucleotides 9, 11, 13, and 14 are combined with another compatible modification, such as a phosphorothioate modification, at one or more of the positions comprising a 2′-OMe modification. Particular examples of such embodiments are described herein, e.g., in certain numbered embodiments set forth above and in modification patterns represented by sequences in the Table of Sequences. In some embodiments, such a modification is combined with one or more, or all, of 2′-OMe modifications at nucleotides 1-4 and/or phosphorothioate modifications at nucleotides 1-3 and 6-10.

In some embodiments, the modified guide region YA sites comprise modifications as described for YA sites above.

Additional embodiments of guide region YA site modifications are set forth in the summary above. Any embodiments set forth elsewhere in this disclosure may be combined to the extent feasible with any of the foregoing embodiments.

Conserved Region YA Site Modifications

Conserved region YA sites 1-10 are illustrated in FIG. 1B. In some embodiments, 1, 2, 3, 4, 5, 6, 7, 8, 9, or 10 conserved region YA sites comprise modifications.

In some embodiments, conserved region YA sites 1, 8, or 1 and 8 comprise YA modifications. In some embodiments, conserved region YA sites 1, 2, 3, 4, and 10 comprise YA modifications. In some embodiments, YA sites 2, 3, 4, 8, and 10 comprise YA modifications. In some embodiments, conserved region YA sites 1, 2, 3, and 10 comprise YA modifications. In some embodiments, YA sites 2, 3, 8, and 10 comprise YA modifications. In some embodiments, YA sites 1, 2, 3, 4, 8, and 10 comprise YA modifications. In some embodiments, 1, 2, 3, 4, 5, 6, 7, or 8 additional conserved region YA sites comprise YA modifications.

In some embodiments, 1, 2, 3, or 4 of conserved region YA sites 2, 3, 4, and 10 comprise YA modifications. In some embodiments, 1, 2, 3, 4, 5, 6, 7, or 8 additional conserved region YA sites comprise YA modifications.

In some embodiments, the modified conserved region YA sites comprise modifications as described for YA sites above.

Additional embodiments of conserved region YA site modifications are set forth in the summary above. Any embodiments set forth elsewhere in this disclosure may be combined to the extent feasible with any of the foregoing embodiments.

### Modifications to Terminal Nucleotides

In some embodiments, the 5′ and/or 3′ terminus regions of a gRNA (e.g., sgRNA, short-sgRNA, dgRNA, or crRNA) are modified.

3′ Terminus Region Modifications

In some embodiments, the terminal (i.e., last) 1, 2, 3, 4, 5, 6, or 7 nucleotides in the 3′ terminus region are modified. Throughout, this modification may be referred to as a “3′ end modification”. In some embodiments, the terminal (i.e., last) 1, 2, 3, 4, 5, 6, or 7 nucleotides in the 3′ terminus region comprise more than one modification. In some embodiments, at least one of the terminal (i.e., last) 1, 2, 3, 4, 5, 6, or 7 nucleotides in the 3′ terminus region are modified. In some embodiments, at least two of the terminal (i.e., last) 1, 2, 3, 4, 5, 6, or 7 nucleotides in the 3′ terminus region are modified. In some embodiments, at least three of the terminal (i.e., last) 1, 2, 3, 4, 5, 6, or 7 nucleotides in the 3′ terminus region are modified. In some embodiments, the modification comprises a PS linkage. In some embodiments, the modification to the 3′ terminus region is a 3′ protective end modification. In some embodiments, the 3′ end modification comprises a 3′ protective end modification.

In some embodiments, the 3′ end modification comprises a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, an ENA, a UNA, a 2′-H (DNA), or combinations thereof.

In some embodiments, the 3′ end modification comprises or further comprises a 2′-O-methyl (2′-O-Me) modified nucleotide.

In some embodiments, the 3′ end modification comprises or further comprises a 2′-fluoro (2′-F) modified nucleotide.

In some embodiments, the 3′ end modification comprises or further comprises a phosphorothioate (PS) linkage between nucleotides.

In some embodiments, the 3′ end modification comprises or further comprises an inverted abasic modified nucleotide.

In some embodiments, the 3′ end modification comprises or further comprises an ENA.

In some embodiments, the 3′ end modification comprises or further comprises a UNA.

In some embodiments, the 3′ end modification comprises or further comprises a 2′-H (DNA).

In some embodiments, the 3′ end modification comprises or further comprises a modification of any one or more of the last 7, 6, 5, 4, 3, 2, or 1 nucleotides. In some embodiments, the 3′ end modification comprises or further comprises one modified nucleotide. In some embodiments, the 3′ end modification comprises or further comprises two modified nucleotides. In some embodiments, the 3′ end modification comprises or further comprises three modified nucleotides. In some embodiments, the 3′ end modification comprises or further comprises four modified nucleotides. In some embodiments, the 3′ end modification comprises or further comprises five modified nucleotides. In some embodiments, the 3′ end modification comprises or further comprises six modified nucleotides. In some embodiments, the 3′ end modification comprises or further comprises seven modified nucleotides.

In some embodiments, the 3′ end modification comprises or further comprises a modification of between 1 and 7 or between 1 and 5 nucleotides.

In some embodiments, the 3′ end modification comprises or further comprises modifications of 1, 2, 3, 4, 5, 6, or 7 nucleotides at the 3′ terminus of the gRNA.

In some embodiments, the 3′ end modification comprises or further comprises modifications of about 1-3, 1-5, 1-6, or 1-7 nucleotides at the 3′ terminus of the gRNA.

In some embodiments, the 3′ end modification comprises or further comprises any one or more of the following: a phosphorothioate (PS) linkage between nucleotides, a 2′-O-Me modified nucleotide, a 2′-O-moe modified nucleotide, a 2′-F modified nucleotide, an inverted abasic modified nucleotide, an ENA, a UNA, and a combination thereof.

In some embodiments, the 3′ end modification comprises or further comprises 1, 2, 3, 4, 5, 6, or 7 PS linkages between nucleotides.

In some embodiments, the 3′ end modification comprises or further comprises at least one 2′-O-Me, 2′-O-moe, inverted abasic, or 2′-F modified nucleotide.

In some embodiments, the 3′ end modification comprises or further comprises one PS linkage, wherein the linkage is between the last and second to last nucleotide. In some embodiments, the 3′ end modification comprises or further comprises two PS linkages between the last three nucleotides. In some embodiments, the 3′ end modification comprises or further comprises four PS linkages between the last four nucleotides.

In some embodiments, the 3′ end modification comprises or further comprises PS linkages between any one or more of the last four nucleotides. In some embodiments, the 3′ end modification comprises or further comprises PS linkages between any one or more of the last five nucleotides. In some embodiments, the 3′ end modification comprises or further comprises PS linkages between any one or more of the last 2, 3, 4, 5, 6, or 7 nucleotides.

In some embodiments, the 3′ end modification comprises or further comprises a modification of one or more of the last 1-7 nucleotides, wherein the modification is a PS linkage, inverted abasic nucleotide, 2′-O-Me, 2′-O-moe, 2′-F, or combinations thereof.

In some embodiments, the 3′ end modification comprises or further comprises a modification to the last nucleotide with 2′-O-Me, 2′-O-moe, 2′-F, or combinations thereof, and an optionally one or two PS linkages to the next nucleotide and/or the first nucleotide of the 3′ tail.

In some embodiments, the 3′ end modification comprises or further comprises a modification to the last and/or second to last nucleotide with 2′-O-Me, 2′-O-moe, 2′-F, or combinations thereof, and optionally one or more PS linkages.

In some embodiments, the 3′ end modification comprises or further comprises a modification to the last, second to last, and/or third to last nucleotides with 2′-O-Me, 2′-O-moe, 2′-F, or combinations thereof, and optionally one or more PS linkages.

In some embodiments, the 3′ end modification comprises or further comprises a modification to the last, second to last, third to last, and/or fourth to last nucleotides with 2′-O-Me, 2′-O-moe, 2′-F, or combinations thereof, and optionally one or more PS linkages.

In some embodiments, the 3′ end modification comprises or further comprises a modification to the last, second to last, third to last, fourth to last, and/or fifth to last nucleotides with 2′-O-Me, 2′-O-moe, 2′-F, or combinations thereof, and optionally one or more PS linkages.

In some embodiments, an sgRNA or short-sgRNA comprising a 3′ end modification comprises or further comprises a 3′ tail, wherein the 3′ tail comprises a modification of any one or more of the nucleotides present in the 3′ tail. In some embodiments, the 3′ tail is fully modified. In some embodiments, the 3′ tail comprises 1, 2, 3, 4, 5, 6, 7, 8, 9, 10, 1-2, 1-3, 1-4, 1-5, 1-6, 1-7, 1-8, 1-9, or 1-10 nucleotides, optionally where any one or more of these nucleotides are modified.

In some embodiments, an sgRNA or short-sgRNA is provided comprising a 3′ end modification, wherein the 3′ end modification comprises the 3′ end modification as shown in any one of SEQ ID Nos: 1-132. In some embodiments, an sgRNA is provided comprising a 3′ protective end modification.

In some embodiments, an sgRNA or short-sgRNA is provided comprising a 3′ end modification, wherein the 3′ end modification comprises (i) a 2′-OMe modified nucleotide at the last nucleotide of the conserved region of an sgRNA or sgRNA (ii) three consecutive 2′O-moe modified nucleotides immediately 5′ to the 2′-OMe modified nucleotide, and (iii) three consecutive PS linkages between the last three nucleotides.

In some embodiments, an sgRNA or short-sgRNA is provided comprising a 3′ end modification, wherein the 3′ end modification comprises (i) five consecutive 2′-OMe modified nucleotides from the last nucleotide of the conserved region of an sgRNA or sgRNA, and (ii) three PS linkages between the last three nucleotides.

In some embodiments, an sgRNA or short-sgRNA is provided comprising a 3′ end modification, wherein the 3′ end modification comprises an inverted abasic modified nucleotide at the last nucleotide of the conserved region of an sgRNA or sgRNA.

In some embodiments, an sgRNA or short-sgRNA is provided comprising a 3′ end modification, wherein the 3′ end modification comprises (i) an inverted abasic modified nucleotide at the last nucleotide of the conserved region of an sgRNA or short-sgRNA, and (ii) three consecutive 2′-OMe modified nucleotides at the last three nucleotides of the conserved region of an sgRNA or short-sgRNA.

In some embodiments, an sgRNA or short-sgRNA is provided comprising a 3′ end modification, wherein the 3′ end modification comprises (i) 15 consecutive 2′-OMe modified nucleotides from the last nucleotide of the conserved region of an sgRNA, (ii) five consecutive 2′-F modified nucleotides immediately 5′ to the 2′-OMe modified nucleotides, and (iii) three PS linkages between the last three nucleotides.

In some embodiments, an sgRNA or short-sgRNA is provided comprising a 3′ end modification, wherein the 3′ end modification comprises (i) alternating 2′-OMe modified nucleotides and 2′-F modified nucleotides at the last 20 nucleotides of the conserved region of an sgRNA or sgRNA, and (ii) three PS linkages between the last three nucleotides.

In some embodiments, an sgRNA or short-sgRNA is provided comprising a 3′ end modification, wherein the 3′ end modification comprises (i) two or three consecutive 2′-OMe modified nucleotides, and (ii) three PS linkages between the last three nucleotides.

In some embodiments, an sgRNA or short-sgRNA is provided comprising a 3′ end modification, wherein the 3′ end modification comprises one PS linkage between the last and next to last nucleotides.

In some embodiments, the sgRNA or short-sgRNA comprises a 5′ end modification and a 3′ end modification.

3′ Tail

In some embodiments, the sgRNA comprises a 3′ terminus comprising a 3′ tail, which follows the 3′ end of the conserved portion of an sgRNA. In some embodiments, the 3′ tail comprises between 1 and about 20 nucleotides, between 1 and about 15 nucleotides, between 1 and about 10 nucleotides, between 1 and about 5 nucleotides, between 1 and about 4 nucleotides, between 1 and about 3 nucleotides, and between 1 and about 2 nucleotides. In some embodiments, the 3′ tail comprises about 1, 2, 3, 4, 5, 6, 7, 8, 9, or 10 nucleotides. In some embodiments, the 3′ tail comprises 1, 2, 3, 4, 5, 6, 7, 8, 9, or 10 nucleotides. In some embodiments, the 3′ tail comprises 1 nucleotide. In some embodiments, the 3′ tail comprises 2 nucleotides. In some embodiments, the 3′ tail comprises 3 nucleotides. In some embodiments, the 3′ tail comprises 4 nucleotides. In some embodiments, the 3′ tail comprises about 1-2, 1-3, 1-4, 1-5, 1-7, 1-10, at least 1-5, at least 1-3, at least 1-4, at least 1-5, at least 1-5, at least 1-7, or at least 1-10 nucleotides.

In some embodiments, the 3′ tail comprising between 1 and 20 nucleotides and follows the 3′ end of the conserved portion of an sgRNA.

In some embodiments, the 3′ tail comprises or further comprises one or more of a protective end modification, a phosphorothioate (PS) linkage between nucleotides, a 2′-O-Me modified nucleotide, a 2′-O-moe modified nucleotide, a 2′-F modified nucleotide, an inverted abasic modified nucleotide, and a combination thereof.

In some embodiments, the 3′ tail comprises or further comprises one or more phosphorothioate (PS) linkages between nucleotides. In some embodiments, the 3′ tail comprises or further comprises one or more 2′-O-Me modified nucleotides. In some embodiments, the 3′ tail comprises or further comprises one or more 2′-O-moe modified nucleotides. In some embodiments, the 3′ tail comprises or further comprises one or more 2′-F modified nucleotide. In some embodiments, the 3′ tail comprises or further comprises one or more an inverted abasic modified nucleotides. In some embodiments, the 3′ tail comprises or further comprises one or more protective end modifications. In some embodiments, the 3′ tail comprises or further comprises a combination of one or more of a phosphorothioate (PS) linkage between nucleotides, a 2′-O-Me modified nucleotide, a 2′-O-moe modified nucleotide, a 2′-F modified nucleotide, and an inverted abasic modified nucleotide.

In some embodiments, the sgRNA does not comprise a 3′ tail.

5′ Terminus Region Modifications

In some embodiments, the 5′ terminus region is modified, for example, the first 1, 2, 3, 4, 5, 6, or 7 nucleotides of the gRNA (e.g., sgRNA, short-sgRNA, or crRNA) are modified. Throughout, this modification may be referred to as a “5′ end modification”. In some embodiments, the first 1, 2, 3, 4, 5, 6, or 7 nucleotides of the 5′ terminus region (i.e., the first 1, 2, 3, 4, 5, 6, or 7 nucleotides from the 5′ end of the 5′ terminus) comprise more than one modification. In some embodiments, at least one of the terminal (i.e., first) 1, 2, 3, 4, 5, 6, or 7 nucleotides from the 5′ end of the 5′ terminus are modified. In some embodiments, at least two of the terminal 1, 2, 3, 4, 5, 6, or 7 nucleotides from the 5′ end of the 5′ terminus are modified. In some embodiments, at least three of the terminal 1, 2, 3, 4, 5, 6, or 7 nucleotides from the 5′ end of the 5′ terminus are modified. In some embodiments, the modification comprises a PS linkage. In some embodiments, the modification to the 5′ terminus region is a 5′ protective end modification. In some embodiments, the 5′ end modification comprises a 5′ protective end modification.

In some embodiments, both the 5′ and 3′ terminus regions of the sgRNA or short-sgRNA are modified (e.g., including the first and last nucleotides of the gRNA). In some embodiments, only the 5′ terminus region of the sgRNA or short-sgRNA is modified. In some embodiments, only the 3′ terminus region (plus or minus a 3′ tail) of the conserved portion of an sgRNA or short-sgRNA is modified.

In some embodiments, the gRNA (e.g., sgRNA, short-sgRNA, or crRNA) comprises modifications at 1, 2, 3, 4, 5, 6, or 7 of the first 7 nucleotides from the 5′ end of the 5′ terminus of the gRNA. In some embodiments, the gRNA (e.g., sgRNA, short-sgRNA, or crRNA) comprises modifications at 1, 2, 3, 4, 5, 6, or 7 of the 7 terminal nucleotides from the 3′ end of the 3′ terminus. In some embodiments, 2, 3, or 4 of the first 4 nucleotides from the 5′ end of the 5′ terminus, and/or 2, 3, or 4 of the terminal 4 nucleotides from the 3′ end of the 3′ terminus are modified. In some embodiments, 2, 3, or 4 of the first 4 nucleotides from the 5′ end of the 5′ terminus are linked with phosphorothioate (PS) bonds.

In some embodiments, the modification to the 5′ terminus and/or 3′ terminus comprises a 2′-O-methyl (2′-O-Me) or 2′-O-(2-methoxyethyl) (2′-O-moe) modification. In some embodiments, the modification comprises a 2′-fluoro (2′-F) modification to a nucleotide. In some embodiments, the modification comprises a phosphorothioate (PS) linkage between nucleotides. In some embodiments, the modification comprises an inverted abasic nucleotide. In some embodiments, the modification comprises a protective end modification. In some embodiments, the modification comprises a more than one modification selected from protective end modification, 2′-O-Me, 2′-O-moe, 2′-fluoro (2′-F), a phosphorothioate (PS) linkage between nucleotides, 2′-H (DNA), an ENA, a UNA, and an inverted abasic nucleotide. In some embodiments, an equivalent modification is encompassed.

In some embodiments, the gRNA (e.g., sgRNA, short-sgRNA, or crRNA) comprises one or more phosphorothioate (PS) linkages between the first one, two, three, four, five, six, or seven nucleotides at the 5′ terminus. In some embodiments, the sgRNA comprises one or more PS linkages between the last one, two, three, four, five, six, or seven nucleotides at the 3′ terminus. In some embodiments, the sgRNA or short-sgRNA comprises one or more PS linkages between both the last one, two, three, four, five, six, or seven nucleotides at the 3′ terminus and the first 1, 2, 3, 4, 5, 6, or 7 nucleotides from the 5′ end of the 5′ terminus. In some embodiments, in addition to PS linkages, the 5′ and 3′ terminal nucleotides may comprise 2′-O-Me, 2′-O-moe, or 2′-F modified nucleotides.

In some embodiments, the sgRNA comprises a 5′ end modification, e.g., wherein the first nucleotide of the guide region is modified. In some embodiments, the sgRNA comprises a 5′ end modification, wherein the first nucleotide of the guide region comprises a 5′ protective end modification.

In some embodiments, the 5′ end modification comprises a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, an ENA, a UNA, a 2′H (DNA), or combinations thereof.

In some embodiments, the 5′ end modification comprises or further comprises a 2′-O-methyl (2′-O-Me) modified nucleotide.

In some embodiments, the 5′ end modification comprises or further comprises a 2′-fluoro (2′-F) modified nucleotide.

In some embodiments, the 5′ end modification comprises or further comprises a phosphorothioate (PS) linkage between nucleotides.

In some embodiments, the 5′ end modification comprises or further comprises an inverted abasic modified nucleotide.

In some embodiments, the 5′ end modification comprises or further comprises an ENA.

In some embodiments, the 5′ end modification comprises or further comprises a UNA.

In some embodiments, the 5′ end modification comprises or further comprises a 2′-H (DNA).

In some embodiments, the 5′ end modification comprises or further comprises a modification of any one or more of nucleotides 1-7 of the guide region of an gRNA (e.g., sgRNA, short-sgRNA, or crRNA). In some embodiments, the 5′ end modification comprises or further comprises one modified nucleotide. In some embodiments, the 5′ end modification comprises or further comprises two modified nucleotides. In some embodiments, the 5′ end modification comprises or further comprises three modified nucleotides. In some embodiments, the 5′ end modification comprises or further comprises four modified nucleotides. In some embodiments, the 5′ end modification comprises or further comprises five modified nucleotides. In some embodiments, the 5′ end modification comprises or further comprises six modified nucleotides. In some embodiments, the 5′ end modification comprises or further comprises seven modified nucleotides.

In some embodiments, the 5′ end modification comprises or further comprises a modification of between 1 and 7, between 1 and 5, between 1 and 4, between 1 and 3, or between 1 and 2 nucleotides.

In some embodiments, the 5′ end modification comprises or further comprises modifications of 1, 2, 3, 4, 5, 6, or 7 nucleotides from the 5′ end. In some embodiments, the 5′ end modification comprises or further comprises modifications of about 1-3, 1-4, 1-5, 1-6, or 1-7 nucleotides from the 5′ end.

In some embodiments, the 5′ end modification comprises or further comprises modifications at the first nucleotide from the 5′ end of the gRNA (e.g., sgRNA, short-sgRNA, or crRNA). In some embodiments, the 5′ end modification comprises or further comprises modifications at the first and second nucleotide from the 5′ end of the gRNA (e.g., sgRNA, short-sgRNA, or crRNA). In some embodiments, the 5′ end modification comprises or further comprises modifications from the first, second, and third nucleotide at the 5′ end of the gRNA (e.g., sgRNA, short-sgRNA, or crRNA). In some embodiments, the 5′ end modification comprises or further comprises modifications at the first, second, third, and fourth nucleotide from the 5′ end of the gRNA (e.g., sgRNA, short-sgRNA, or crRNA). In some embodiments, the 5′ end modification comprises or further comprises modifications at the first, second, third, fourth, and fifth nucleotide from the 5′ end of the gRNA (e.g., sgRNA, short-sgRNA, or crRNA). In some embodiments, the 5′ end modification comprises or further comprises modifications at the first, second, third, fourth, fifth, and sixth nucleotide from the 5′ end of the gRNA (e.g., sgRNA, short-sgRNA, or crRNA). In some embodiments, the 5′ end modification comprises or further comprises modifications from the first, second, third, fourth, fifth, sixth, and seventh nucleotide at the 5′ end of the gRNA (e.g., sgRNA, short-sgRNA, or crRNA).

In some embodiments, the 5′ end modification comprises or further comprises a phosphorothioate (PS) linkage between nucleotides, and/or a 2′-O-Me modified nucleotide, and/or a 2′-O-moe modified nucleotide, and/or a 2′-F modified nucleotide, and/or an inverted abasic modified nucleotide, and/or combinations thereof.

In some embodiments, the 5′ end modification comprises or further comprises 1, 2, 3, 4, 5, 6, and/or 7 PS linkages between nucleotides. In some embodiments, the 5′ end modification comprises or further comprises about 1-2, 1-3, 1-4, 1-5, 1-6, or 1-7 PS linkages between nucleotides.

In some embodiments, the 5′ end modification comprises or further comprises at least one PS linkage, wherein if there is one PS linkage, the linkage is between nucleotides 1 and 2 of the guide region.

In some embodiments, the 5′ end modification comprises or further comprises at least two PS linkages, and the linkages are between nucleotides 1 and 2, and 2 and 3 of the guide region.

In some embodiments, the 5′ end modification comprises or further comprises PS linkages between any one or more of nucleotides 1 and 2, 2 and 3, and 3 and 4 of the guide region.

In some embodiments, the 5′ end modification comprises or further comprises PS linkages between any one or more of nucleotides 1 and 2, 2 and 3, 3 and 4, and 4 and 5 of the guide region.

In some embodiments, the 5′ end modification comprises or further comprises PS linkages between any one or more of nucleotides 1 and 2, 2 and 3, 3 and 4, 4 and 5, and 5 and 6 of the guide region.

In some embodiments, the 5′ end modification comprises or further comprises PS linkages between any one or more of nucleotides 1 and 2, 2 and 3, 3 and 4, 4 and 5, 5 and 6, and 7 and 8 of the guide region.

In some embodiments, the 5′ end modification comprises or further comprises a modification of one or more of nucleotides 1-7 of the guide region, wherein the modification is a PS linkage, inverted abasic nucleotide, 2′-O-Me, 2′-O-moe, 2′-F, and/or combinations thereof.

In some embodiments, the 5′ end modification comprises or further comprises a modification to the first nucleotide of the guide region with 2′-O-Me, 2′-O-moe, 2′-F, or combinations thereof, and an optional PS linkage to the next nucleotide;

In some embodiments, the 5′ end modification comprises or further comprises a modification to the first and/or second nucleotide of the guide region with 2′-O-Me, 2′-O-moe, 2′-F, or combinations thereof, and optionally one or more PS linkages between the first and second nucleotide and/or between the second and third nucleotide.

In some embodiments, the 5′ end modification comprises or further comprises a modification to the first, second, and/or third nucleotides of the variable region with 2′-O-Me, 2′-O-moe, 2′-F, or combinations thereof, and optionally one or more PS linkages between the first and second nucleotide, between the second and third nucleotide, and/or between the third and the fourth nucleotide.

In some embodiments, the 5′ end modification comprises or further comprises a modification to the first, second, third, and/or fourth nucleotides of the variable region with 2′-O-Me, 2′-O-moe, 2′-F, or combinations thereof, and optionally one or more PS linkages between the first and second nucleotide, between the second and third nucleotide, between the third and the fourth nucleotide, and/or between the fourth and the fifth nucleotide.

In some embodiments, the 5′ end modification comprises or further comprises a modification to the first, second, third, fourth, and/or fifth nucleotides of the variable region with 2′-O-Me, 2′-O-moe, 2′-F, or combinations thereof, and optionally one or more PS linkages between the first and second nucleotide, between the second and third nucleotide, between the third and the fourth nucleotide, between the fourth and the fifth nucleotide, and/or between the fifth and the sixth nucleotide.

In some embodiments, a gRNA (e.g., sgRNA, short-sgRNA, or crRNA) is provided comprising a 5′ end modification, wherein the 5′ end modification comprises a 5′ end modification as shown in any one of SEQ ID Nos: 401-532, 1001, or 1007-1132. In some embodiments, a gRNA (e.g., sgRNA, short-sgRNA, or crRNA) is provided comprising a 5′ end modification, wherein the 5′ end modification comprises a 5′ end modification as shown in nucleotides 1-3 of any one of SEQ ID Nos: 401-532, 1001, or 1007-1132. In some embodiments, a gRNA (e.g., sgRNA, short-sgRNA, or crRNA) is provided comprising a 5′ end modification, wherein the 5′ end modification comprises a 5′ end modification as shown in nucleotides 1-4 of any one of SEQ ID Nos: 401-532, 1001, or 1007-1132. In some embodiments, a gRNA (e.g., sgRNA, short-sgRNA, or crRNA) is provided comprising a 5′ end modification, wherein the 5′ end modification comprises a 5′ end modification as shown in nucleotides 1-5 of any one of SEQ ID Nos: 401-532, 1001, or 1007-1132. In some embodiments, a gRNA (e.g., sgRNA, short-sgRNA, or crRNA) is provided comprising a 5′ end modification, wherein the 5′ end modification comprises a 5′ end modification as shown in nucleotides 1-6 of any one of SEQ ID Nos: 401-532, 1001, or 1007-1132. In some embodiments, a gRNA (e.g., sgRNA, short-sgRNA, or crRNA) is provided comprising a 5′ end modification, wherein the 5′ end modification comprises a 5′ end modification as shown in nucleotides 1-7 of any one of SEQ ID Nos: 401-532, 1001, or 1007-1132.

In some embodiments, the gRNA (e.g., sgRNA, short-sgRNA, or crRNA) comprises a 5′ end modification comprising a 5′ protective end modification. In some embodiments, a gRNA (e.g., sgRNA, short-sgRNA, or crRNA) is provided comprising a 5′ end modification, wherein the 5′ end modification comprises 2′-OMe modified nucleotides at nucleotides 1, 2, and 3 of the guide region.

In some embodiments, a gRNA (e.g., sgRNA, short-sgRNA, or crRNA) is provided comprising a 5′ end modification, wherein the 5′ end modification comprises 2′-OMe modified nucleotides at nucleotides 1, 2, and 3 of the guide region and PS linkages between nucleotides 1 and 2, 2 and 3, and 3 and 4 of the guide region.

In some embodiments, a gRNA (e.g., sgRNA, short-sgRNA, or crRNA) is provided comprising a 5′ end modification, wherein the 5′ end modification comprises 2′-OMe modified nucleotides at nucleotides 1, 2, 3, 4, and 5 of the guide region.

In some embodiments, a gRNA (e.g., sgRNA, short-sgRNA, or crRNA) is provided comprising a 5′ end modification, wherein the 5′ end modification comprises 2′-OMe modified nucleotides at nucleotides 1, 2, 3, 4, and 5 of the guide region and PS linkages between nucleotides 1 and 2, 2 and 3, 3 and 4, 4 and 5, and 5 and 6 of the guide region.

In some embodiments, a gRNA (e.g., sgRNA, short-sgRNA, or crRNA) is provided comprising a 5′ end modification, wherein the 5′ end modification comprises 2′O-moe modified nucleotides at nucleotides 1, 2, and 3 of the guide region.

In some embodiments, a gRNA (e.g., sgRNA, short-sgRNA, or crRNA) is provided comprising a 5′ end modification, wherein the 5′ end modification comprises 2′O-moe modified nucleotides at nucleotides 1, 2, and 3 of the guide region and PS linkages between nucleotides 1 and 2, 2 and 3, and 3 and 4 of the guide region.

In some embodiments, a gRNA (e.g., sgRNA, short-sgRNA, or crRNA) is provided comprising a 5′ end modification, wherein the 5′ end modification comprises an inverted abasic modified nucleotide at nucleotide 1 of the guide region.

In some embodiments, agRNA (e.g., sgRNA, short-sgRNA, or crRNA) is provided comprising a 5′ end modification, wherein the 5′ end modification comprises an inverted abasic modified nucleotide at nucleotide 1 of the guide region and 2′-OMe modified nucleotides at nucleotides 1, 2, and 3 of the guide region.

In some embodiments, agRNA (e.g., sgRNA, short-sgRNA, or crRNA) is provided comprising a 5′ end modification, wherein the 5′ end modification comprises an inverted abasic modified nucleotide at nucleotide 1 of the guide region, 2′-OMe modified nucleotides at nucleotides 1, 2, and 3 of the guide region, and PS linkages between nucleotides 1 and 2, 2 and 3, 3 and 4, 4 and 5, and 5 and 6 of the guide region.

In some embodiments, an sgRNA or short-sgRNA is provided comprising a 5′ end modification and a 3′ end modification. Any of the 5′ end modifications discussed above and/or otherwise disclosed herein can be combined with a 3′ end modification, such as a 3′ end modification represented in the Table of Sequences and/or discussed below.

In some embodiments, the sgRNA or short-sgRNA comprises modified nucleotides at the 5′ and 3′ terminus, and modified nucleotides in one or more other regions described in Table 3.

In some embodiments, the sgRNA or short-sgRNA comprises modified nucleotides that are not at the 5′ or 3′ ends. Exemplary patterns of modifications are described below and in Table 1.

### Modifications to Stabilize Secondary Structures

In some embodiments, an gRNA (e.g., sgRNA, short-sgRNA, or crRNA) comprises a modification that stabilizes a secondary structure, e.g. a duplex region. Increased stability of a secondary structure can be determined empirically, e.g., by a melting temperature analysis. To simplify the analysis, the secondary structure element sought to be stabilized can be tested in isolation from the rest of the sgRNA structure. Increased stability of a secondary structure can also be determined by a reduction in accessibility of an endonucleolytic cleavage site (e.g., YA site) wherein the modification does not alter the endonucleolytic cleavage site primary structure but does occur in or affect a secondary structure comprising the endonucleolytic cleavage site. This is referred to as having a distal effect on the endonucleolytic cleavage site. In some embodiments, the endonucleolytic cleavage site is in the lower stem. In some embodiments, the endonucleolytic cleavage site is conserved region YA site 1. In some embodiments, the endonucleolytic cleavage site is conserved region YA site 2. In some embodiments, the endonucleolytic cleavage site is conserved region YA site 3. In some embodiments, the endonucleolytic cleavage site is conserved region YA site 10. In some embodiments, the modification is a bicyclic ribose analog modification, such as, a locked nucleic acid (LNA) or LNA-like modification. In some embodiments, the modification is an ENA modification. In some embodiments, nucleotide LS8 comprises a modification that stabilizes a secondary structure. In some embodiments, nucleotide LS11 comprises a modification that stabilizes a secondary structure. In some embodiments, one or both of nucleotides LS8 and LS11 collectively comprise one or more modifications (e.g., 2 modifications), such as ENA modifications, that stabilize a secondary structure. See discussion of G10008 and G10038 in the examples.

### Additional Modifications

In some embodiments, a gRNA (e.g., sgRNA, short-sgRNA, or crRNA) comprises modifications and/or unmodified nucleotides at at least 15 of nucleotides 1-20 from the 5′ end of the 5′ terminus that match the modification pattern at nucleotides 1-20 of a gRNA described herein, e.g., in Table 1. In some embodiments, a gRNA (e.g., sgRNA, short-sgRNA, or crRNA) comprises modifications and/or unmodified nucleotides at at least 16 of nucleotides 1-20 from the 5′ end of the 5′ terminus that match the modification pattern at nucleotides 1-20 of a gRNA described herein, e.g., in Table 1. In some embodiments, a gRNA (e.g., sgRNA, short-sgRNA, or crRNA) comprises modifications and/or unmodified nucleotides at at least 17 of nucleotides 1-20 from the 5′ end of the 5′ terminus that match the modification pattern at nucleotides 1-20 of a gRNA described herein, e.g., in Table 1. In some embodiments, a gRNA (e.g., sgRNA, short-sgRNA, or crRNA) comprises modifications and/or unmodified nucleotides at at least 18 of nucleotides 1-20 from the 5′ end of the 5′ terminus that match the modification pattern at nucleotides 1-20 of a gRNA described herein, e.g., in Table 1. In some embodiments, a gRNA (e.g., sgRNA, short-sgRNA, or crRNA) comprises modifications and/or unmodified nucleotides at at least 19 of nucleotides 1-20 from the 5′ end of the 5′ terminus that match the modification pattern at nucleotides 1-20 of a gRNA described herein, e.g., in Table 1. In some embodiments, a gRNA (e.g., sgRNA, short-sgRNA, or crRNA) comprises modifications and/or unmodified nucleotides at nucleotides 1-20 from the 5′ end of the 5′ terminus that match the modification pattern at nucleotides 1-20 of a gRNA described herein, e.g., in Table 1. In some embodiments, a gRNA comprises a modification pattern that matches at least 75% of the modification pattern of a gRNA described herein, e.g., in Table 1. In some embodiments, a gRNA comprises a modification pattern that matches at least 80% of the modification pattern of a gRNA described herein, e.g., in Table 1. In some embodiments, a gRNA comprises a modification pattern that matches at least 85% of the modification pattern of a gRNA described herein, e.g., in Table 1. In some embodiments, a gRNA comprises a modification pattern that matches at least 90% of the modification pattern of a gRNA described herein, e.g., in Table 1. In some embodiments, a gRNA comprises a modification pattern that matches at least 95% of the modification pattern of a gRNA described herein, e.g., in Table 1. In some embodiments, a gRNA comprises a modification pattern that matches at least 98% of the modification pattern of a gRNA described herein, e.g., in Table 1. In some embodiments, a gRNA comprises a modification pattern that matches the modification pattern of a gRNA described herein, e.g., in Table 1. In some embodiments, an sgRNA or short-sgRNA comprises modifications in any one or more of the regions shown as modified in Table 1. In some embodiments, an sgRNA or short-sgRNA comprises modifications at any of the positions shown as modified in Table 1. In some embodiments, an sgRNA or short-sgRNA comprises any of the modifications shown in Table 1. Additional modifications are set forth in the summary section above, which may be combined to the extent feasible with modifications disclosed elsewhere herein, such as YA site modifications.

In some embodiments, an sgRNA or short-sgRNA is provided comprising an upper stem modification, wherein the upper stem modification comprises a modification to any one or more of US1-US12 in the upper stem region.

In some embodiments, an sgRNA or short-sgRNA is provided comprising an upper stem modification, wherein the upper stem modification comprises a modification of at least 1, 2, 3, 4, 5, 6, 7, 8, 9, 10, 11, or all 12 nucleotides in the upper stem region.

In some embodiments, an sgRNA or short-sgRNA is provided comprising an upper stem modification, wherein the upper stem modification comprises a modification of about 1-2, 1-3, 1-4, 1-5, 1-6, 1-7, 1-8, 1-9, 1-10, or 1-12 nucleotides in the upper stem region.

In some embodiments, an sgRNA or short-sgRNA is provided comprising an upper stem modification, wherein the upper stem modification comprises 1, 2, 3, 4, or 5 YA modifications in a YA site. In some embodiments, an sgRNA or short-sgRNA is provided comprising an upper stem modification, wherein the upper stem modification comprises at least 1, 2, 3, 4, or 5 YA modifications. In some embodiments, an sgRNA or short-sgRNA is provided comprising an upper stem modification, wherein the upper stem modification comprises one YA modification. In some embodiments, an sgRNA or short-sgRNA is provided comprising an upper stem modification, wherein the upper stem modification comprises 2 YA modifications. In some embodiments, the upper stem modification comprises 3 YA modifications. In some embodiments, one or more YA modifications are in a YA site. In some embodiments, one or more YA modifications are distal to a YA site.

In some embodiments, an sgRNA or short-sgRNA is provided comprising an upper stem modification, wherein the upper stem modification comprises a 2′-O-Me modified nucleotide. In some embodiments, an sgRNA or short-sgRNA is provided comprising an upper stem modification, wherein the upper stem modification comprises a 2′-O-moe modified nucleotide. In some embodiments, an sgRNA or short-sgRNA is provided comprising an upper stem modification, wherein the upper stem modification comprises a 2′-F modified nucleotide.

In some embodiments, an sgRNA or short-sgRNA is provided comprising an upper stem modification, wherein the upper stem modification comprises a 2′-O-Me modified nucleotide, a 2′-O-moe modified nucleotide, a 2′-F modified nucleotide, and/or combinations thereof.

In some embodiments, the sgRNA or short-sgRNA comprises an upper stem modification as shown in any one of the sequences in Table 1. In some embodiments, such an upper stem modification is combined with a 5′ protective end modification, e.g. as shown for the corresponding sequence in Table 1. In some embodiments, such an upper stem modification is combined with a 3′ protective end modification, e.g. as shown for the corresponding sequence in Table 1. In some embodiments, such an upper stem modification is combined with 5′ and 3′ end modifications as shown for the corresponding sequence in Table 1.

In some embodiments, the sgRNA or short-sgRNA comprises a 5′ end modification and an upper stem modification. In some embodiments, the sgRNA or short-sgRNA comprises a 3′ end modification and an upper stem modification. In some embodiments, the sgRNA or short-sgRNA comprises a 5′ end modification, a 3′ end modification and an upper stem modification.

In some embodiments, the sgRNA or short-sgRNA comprises a modification in the hairpin region. In some embodiments, the hairpin region modification is in hairpin 1. In some embodiments, the hairpin region modification is in hairpin 2. In some embodiments, modifications are within hairpin 1 and 2, optionally wherein the “n” between hairpin 1 and 2 is also modified. In some embodiments, the hairpin region modification comprises at least one modified nucleotide selected from a 2′H modified nucleotide (DNA), PS modified nucleotide, a YA modification, a 2′-O-methyl (2′-O-Me) modified nucleotide, a 2′-fluoro (2′-F) modified nucleotide, and/or combinations thereof.

In some embodiments, an sgRNA or short-sgRNA is provided comprising a hairpin modification, wherein the hairpin modification comprises 1, 2, or 3 YA modifications in a YA site. In some embodiments, an sgRNA or short-sgRNA is provided comprising a hairpin modification, wherein the hairpin modification comprises at least 1, 2, 3, 4, 5, or 6 YA modifications. In some embodiments, an sgRNA or short-sgRNA is provided comprising a hairpin modification, wherein the hairpin modification comprises one YA modification. In some embodiments, an sgRNA or short-sgRNA is provided comprising a hairpin modification, wherein hairpin modification comprises 2 YA modifications. In some embodiments, the hairpin modification comprises 3 YA modifications. In some embodiments, one or more YA modifications are in a YA site. In some embodiments, one or more YA modifications are distal to a YA site.

In some embodiments, the hairpin modification comprises or further comprises a 2′-O-methyl (2′-O-Me) modified nucleotide.

In some embodiments, the hairpin modification comprises or further comprises a 2′-fluoro (2′-F) modified nucleotide.

In some embodiments, the sgRNA or short-sgRNA comprises a 3′ end modification, and a modification in the hairpin region.

In some embodiments, the sgRNA or short-sgRNA comprises a 5′ end modification, and a modification in the hairpin region.

In some embodiments, the sgRNA or short-sgRNA comprises an upper stem modification, and a modification in the hairpin region.

In some embodiments, the sgRNA or short-sgRNA comprises a hairpin modification as shown in any one of the sequences in Table 1. In some embodiments, such a hairpin modification is combined with a 5′ end modification as shown for the corresponding sequence in Table 1. In some embodiments, such a hairpin modification is combined with a 3′ end modification as shown for the corresponding sequence in Table 1. In some embodiments, such an hairpin modification is combined with 5′ and 3′ end modifications as shown for the corresponding sequence in Table 1.

In some embodiments, the sgRNA or short-sgRNA comprises a 3′ end modification, a modification in the hairpin region, an upper stem modification, and a 5′ end modification.

In some embodiments, the sgRNA or short-sgRNA comprising one or more modifications of YA sites is a short-sgRNA as described herein, e.g., comprising a hairpin region that lacks at least 5-10 nucleotides, e.g., as defined herein or relative to the hairpin region shown in Table 2. Such an sgRNA may have any of the features set forth herein with respect to an sgRNA, e.g., in the summary and in the detailed description section regarding short-sgRNAs above.

Exemplary Modified sgRNAs

In some embodiments, the sgRNAs described herein comprise or consist of any of the sequences shown in Table 1. Further, sgRNAs are encompassed that comprise the modifications of any of the sequences shown in Table 1, and identified therein by SEQ ID No. That is, the nucleotides may be the same or different, but the modification pattern shown may be the same or similar to a modification pattern of a guide sequence of Table 1. A modification pattern includes the relative position and identity of modifications of the sgRNA (e.g. 5′ terminus region, lower stem region, bulge region, upper stem region, nexus region, hairpin 1 region, hairpin 2 region, 3′ tail region).

In some embodiments, the modification pattern contains at least 50%, 55%, 60%, 70%, 75%, 80%, 85%, 90%, 95%, 96%, 97%, 98%, and 99% of the modifications of any one of the sequences shown in the sequence column of Table 1, or over one or more regions of the sequence. In some embodiments, the modification pattern is at least 50%, 55%, 60%, 70%, 75%, 80%, 85%, 90%, 95%, 96%, 97%, 98%, and 99% identical to the modification pattern of any one of the sequences shown in the sequence column of Table 1. In some embodiments, the modification pattern is at least 50%, 55%, 60%, 70%, 75%, 80%, 85%, 90%, 95%, 96%, 97%, 98%, and 99% identical over 1, 2, 3, 4, 5, 6, 7, or 8 regions of the sequence shown in Table 1, e.g., a 5′ terminus region, lower stem region, bulge region, upper stem region, nexus region, hairpin 1 region, hairpin 2 region, and/or 3′ terminus region.

For example, in some embodiments, an sgRNA is encompassed wherein the modification pattern is least 50%, 55%, 60%, 70%, 75%, 80%, 85%, 90%, 95%, 96%, 97%, 98%, and 99% identical to the modification pattern of a sequence over the 5′ terminus region. In some embodiments, an sgRNA is encompassed wherein the modification pattern is least 50%, 55%, 60%, 70%, 75%, 80%, 85%, 90%, 95%, 96%, 97%, 98%, and 99% identical over the lower stem. In some embodiments, an sgRNA is encompassed wherein the modification pattern is least 50%, 55%, 60%, 70%, 75%, 80%, 85%, 90%, 95%, 96%, 97%, 98%, and 99% identical over the bulge. In some embodiments, an sgRNA is encompassed wherein the modification pattern is least 50%, 55%, 60%, 70%, 75%, 80%, 85%, 90%, 95%, 96%, 97%, 98%, and 99% identical over the upper stem. In some embodiments, an sgRNA is encompassed wherein the modification pattern is least 50%, 55%, 60%, 70%, 75%, 80%, 85%, 90%, 95%, 96%, 97%, 98%, and 99% identical over the nexus. In some embodiments, an sgRNA is encompassed wherein the modification pattern is least 50%, 55%, 60%, 70%, 75%, 80%, 85%, 90%, 95%, 96%, 97%, 98%, and 99% identical over the hairpin 1. In some embodiments, an sgRNA is encompassed wherein the modification pattern is least 50%, 55%, 60%, 70%, 75%, 80%, 85%, 90%, 95%, 96%, 97%, 98%, and 99% identical over the hairpin 2. In some embodiments, an sgRNA is encompassed wherein the modification pattern is least 50%, 55%, 60%, 70%, 75%, 80%, 85%, 90%, 95%, 96%, 97%, 98%, and 99% identical over the 3′ terminus. In some embodiments, the modification pattern differs from the modification pattern of a sequence of Table 1, or a region (e.g. 5′ terminus, lower stem, bulge, upper stem, nexus, hairpin 1, hairpin 2, 3′ terminus) of such a sequence, at 0, 1, 2, 3, 4, 5, or 6 nucleotides. In some embodiments, the sgRNA comprises modifications that differ from the modifications of a sequence of Table 1, at 0, 1, 2, 3, 4, 5, or 6 nucleotides. In some embodiments, the sgRNA comprises modifications that differ from modifications of a region (e.g. 5′ terminus, lower stem, bulge, upper stem, nexus, hairpin 1, hairpin 2, 3′ terminus) of a sequence of Table 1, at 0, 1, 2, 3, 4, 5, or 6 nucleotides.

In some embodiments, the sgRNA comprises a 2′-O-methyl (2′-O-Me) modified nucleotide. In some embodiments, the sgRNA comprises a 2′-O-(2-methoxyethyl) (2′-O-moe) modified nucleotide. In some embodiments, the sgRNA comprises a 2′-fluoro (2′-F) modified nucleotide. In some embodiments, the sgRNA comprises a phosphorothioate (PS) bond between nucleotides. In some embodiments, the sgRNA comprises a YA modification.

In some embodiments, the sgRNA comprises a 5′ end modification, a 3′ end modification, or 5′ and 3′ end modification and further comprises a YA modification. In some embodiments, the 5′ end modification comprises a protective end modification. In some embodiments, the 5′ end modification comprises a phosphorothioate (PS) bond between nucleotides. In some embodiments, the 5′ end modification comprises a 2′-O-methyl (2′-O-Me), 2′-O-(2-methoxyethyl) (2′-O-moe), and/or 2′-fluoro (2′-F) modified nucleotide. In some embodiments, the 5′ end modification comprises at least one phosphorothioate (PS) bond and one or more of a 2′-O-methyl (2′-O-Me), 2′-O-(2-methoxyethyl) (2′-O-moe), and/or 2′-fluoro (2′-F) modified nucleotide. The end modification may comprise a phosphorothioate (PS), 2′-O-methyl (2′-O-Me), 2′-O-(2-methoxyethyl) (2′-O-moe), and/or 2′-fluoro (2′-F) modification. Equivalent end modifications are also encompassed by embodiments described herein. In some embodiments, the sgRNA comprises an end modification in combination with a modification of one or more regions of the sgRNA.

Modified sgRNAs comprising combinations of 5′ end modifications, 3′ end modifications, upper stem modifications, hairpin modifications, and 3′ terminus modifications, as described above, are encompassed. Exemplary modified sgRNAs are described below.

In some embodiments, an sgRNA is provided comprising or consisting of any one of the sequences described in SEQ ID Nos: 401-535, 601, 607-732, 801, 807-932, 1001, or 1007-1132.

In some embodiments, an sgRNA is provided comprising any one of the modified sequences of SEQ ID Nos: 601 or 607-732, wherein the sgRNA further comprises a guide region that is complementary to a target sequence, and directs a Cas9 to its target for cleavage. In some instances, an sgRNA is provided comprising nucleic acids having at least 99, 98, 97, 96, 95, 94, 93, 92, 91, 90, 85, 80, 75, or 70% identity to the nucleic acids of any one of SEQ ID Nos: 401-535, 601, 607-732, 801, 807-932, 1001, or 1007-1132, wherein the modification pattern is identical to the modification pattern shown in the reference sequence identifier in Table 1. In some embodiments, the sgRNA further comprises three phosphorothioate (PS) bonds linking the first four nucleotides at the 5′ terminus and three PS bonds linking the last four nucleotides at the 3′ terminus.

In some embodiments, the sgRNA comprises modifications at 1, 2, 3, or 4 of the first 4 nucleotides at its 5′ end. In some embodiments, the first three or four nucleotides at the 5′ terminus, and the last three or four nucleotides at the 3′ terminus are modified. In some embodiments, the first four nucleotides at the 5′ end, and the last four nucleotides at the 3′ terminus are linked with phosphorothioate (PS) bonds. In some embodiments, the modification comprises 2′-O-Me. In some embodiments, the modification comprises 2′-F. In some embodiments, the modification comprises 2′-O-moe.

In some embodiments, the sgRNA comprises, if the nucleotide mentioned is present in the sgRNA, modifications at 1, 2, 3, or 4 of the first 4 nucleotides at the 5′ end. In some embodiments, the sgRNA comprises modifications at 1, 2, 3, or 4 of the last 4 nucleotides at the 3′ end (3′ tail or conserved portion of an sgRNA). In some embodiments, the first four nucleotides at the 5′ terminus and the last four nucleotides at the 3′ terminus are linked with a PS bond, and the first three nucleotides at the 5′ terminus and the last three nucleotides at the 3′ terminus comprise 2′-O-Me or 2′-O-moe modifications.

In some embodiments, the first four nucleotides at the 5′ terminus and the last four nucleotides at the 3′ terminus are linked with a PS bond, and the first three nucleotides at the 5′ terminus and the last three nucleotides at the 3′ terminus comprise 2′-F modifications.

In some embodiments, an sgRNA is provided, if the nucleotide mentioned is present in the sgRNA, wherein LS1, LS6, LS7, LS8, LS11, and LS12 are modified with 2′-O-Me. In some embodiments, each of the nucleotides in the bulge region of the sgRNA are modified with 2′-O-Me. In some embodiments, each of the nucleotides in the upper stem region of the sgRNA are modified with 2′-O-Me. In some embodiments, N16, N17, and N18 in the nexus region of the sgRNA are modified with 2′-O-Me. In some embodiments, each of the nucleotides in the hairpin 1 region of the sgRNA are modified with 2′-O-Me. In some embodiments, each of the nucleotides in the hairpin 2 region of the sgRNA are modified with 2′-O-Me.

In some embodiments, the sgRNA comprises 2′-O-Me modified nucleotides at the following nucleotides: the first three nucleotides at the 5′ terminus; LS1, LS6, LS7, LS8, LS11, and LS12; B1 and B2 in the bulge region; each of the nucleotides in the upper stem region of the sgRNA; N16, N17, and N18 in the nexus region; each of the nucleotides in the hairpin 1 region; each of the nucleotides in the hairpin 2 region; and last four nucleotides at the 3′ terminus.

In some embodiments, the sgRNA further comprises three phosphorothioate (PS) bonds linking the first four nucleotides at the 5′ terminus and three PS bonds linking the last four nucleotides at the 3′ terminus. In some embodiments, the sgRNA further comprises 2′-O-Me or 2′-F modified nucleic acids at the first three nucleotides at the 5′ terminus, and 2′-O-Me or 2′-F modified nucleic acids at the last four nucleotides at the 3′ terminus. In some embodiments, LS9 and LS10 are modified with 2′-F. In some embodiments, N15, N16, N17, and N18 are modified with 2′-F. In some embodiments, H2-9, H2-10, H2-11, H2-12, H2-13, HS-14, and H2-15 are modified with 2′-F. In some embodiments, the second to last, third to last, and fourth to last nucleotides at the 3′ terminus are modified with 2′-F.

In some embodiments, sgRNA is provided comprising 2′-F modified nucleic acids at the following nucleotides: LS9 and LS10 in the lower stem region; N15, N16, N17, and N18 in the nexus region; and H2-9, H2-10, H2-11, H2-12, H2-13, HS-14, and H2-15 in the hairpin 2 region. In some embodiments, the sgRNA further comprises 2′-F modified nucleotides at the second to last, third to last, and fourth to last nucleotides at the 3′ terminus. In some embodiments, the sgRNA further comprises three phosphorothioate (PS) bonds linking the first four nucleotides at the 5′ terminus and three PS bonds linking the last four nucleotides at the 3′ terminus. In some embodiments, the sgRNA further comprises 2′-O-Me or 2′-F modified nucleic acids at the first three nucleotides at the 5′ terminus, and 2′-O-Me or 2′-F modified nucleic acids at three of the last four nucleotides at the 3′ terminus.

In some embodiments, an sgRNA is provided comprising: 2′-O-Me modified nucleotides at the first three nucleotides at the 5′ terminus; 2′-O-Me modified nucleotides at LS1 and LS6; 2′-O-Me modified nucleotides at US1-US12; 2′-O-Me modified nucleotides at H1-1-H1-12; a 2′-O-Me modified nucleotide between Hairpin 1 and Hairpin 2; 2′-O-Me modified nucleotides at H2-1-H2-15; and 2′-O-Me modified nucleotides at the last four nucleotides at the 3′ terminus. In some embodiments, the sgRNA further comprises three phosphorothioate (PS) bonds linking the first four nucleotides at the 5′ terminus and three PS bonds linking the last four nucleotides at the 3′ terminus.

In some embodiments, an sgRNA is provided comprising 2′-O-Me modified nucleotides at the first three nucleotides at the 5′ terminus; 2′-F modified nucleotides at LS1-LS6; 2′-O-Me modified nucleotides at US1-US12; 2′-O-Me modified nucleotides at H1-1-H1-12; a 2′-O-Me modified nucleotide at “n” between Hairpin 1 and Hairpin 2; 2′-O-Me modified nucleotides at H2-1-H2-15; and 2′-O-Me modified nucleotides at the last four nucleotides at the 3′ terminus. In some embodiments, the sgRNA further comprises three phosphorothioate (PS) bonds linking the first four nucleotides at the 5′ terminus and three PS bonds linking the last four nucleotides at the 3′ terminus.

In some embodiments, an sgRNA is provided comprising 2′-O-Me modified nucleotides at the first three nucleotides at the 5′ terminus; 2′-F modified nucleotides at LS2-LS5; 2′-O-Me modified nucleotides at LS1 and LS6; 2′-O-Me modified nucleotides at US1-US12; 2′-O-Me modified nucleotides at H1-1-H1-12; a 2′-O-Me modified nucleotide at “n” between Hairpin 1 and Hairpin 2; 2′-O-Me modified nucleotides at H2-1-H2-15; and 2′-O-Me modified nucleotides at the last four nucleotides at the 3′ terminus. In some embodiments, the sgRNA further comprises three phosphorothioate (PS) bonds linking the first four nucleotides at the 5′ terminus and three PS bonds linking the last four nucleotides at the 3′ terminus.

In some embodiments, an sgRNA is provided comprising 2′-O-Me modified nucleotides at the first three nucleotides at the 5′ terminus; 2′-O-Me modified nucleotides at US1-US12; 2′-O-Me modified nucleotides at LS7, LS8, LS11, and LS12; 2′-O-Me modified nucleotides at H1-1-H1-12; a 2′-O-Me modified nucleotide at “n” between Hairpin 1 and Hairpin 2; 2′-O-Me modified nucleotides at H2-1-H2-15; and 2′-O-Me modified nucleotides at the last four nucleotides at the 3′ terminus. In some embodiments, the sgRNA further comprises three phosphorothioate (PS) bonds linking the first four nucleotides at the 5′ terminus and three PS bonds linking the last four nucleotides at the 3′ terminus.

In some embodiments, an sgRNA is provided comprising 2′-O-Me modified nucleotides at the first three nucleotides at the 5′ terminus; 2′-O-Me modified nucleotides at US1-US12; 2′-O-Me modified nucleotides at LS8, LS10, and LS12; 2′-O-F modified nucleotides at LS7, LS9, and LS11; 2′-O-Me modified nucleotides at H1-1-H1-12; a 2′-O-Me modified nucleotide between Hairpin 1 and Hairpin 2; 2′-O-Me modified nucleotides at H2-1-H2-15; and 2′-O-Me modified nucleotides at the last four nucleotides at the 3′ terminus. In some embodiments, the sgRNA further comprises three phosphorothioate (PS) bonds linking the first four nucleotides at the 5′ terminus and three PS bonds linking the last four nucleotides at the 3′ terminus.

In some embodiments, an sgRNA is provided comprising 2′-O-Me modified nucleotides at the first three nucleotides at the 5′ terminus; 2′-O-Me modified nucleotides at LS1, LS6, LS7, LS8, LS11, and LS12; 2′-O-Me modified nucleotides at US1-US12; 2′-O-Me modified nucleotides at H1-1-H1-12; a 2′-O-Me modified nucleotide between Hairpin 1 and Hairpin 2; 2′-O-Me modified nucleotides at H2-1-H2-15; and 2′-O-Me modified nucleotides at the last four nucleotides at the 3′ terminus. In some embodiments, the sgRNA further comprises three phosphorothioate (PS) bonds linking the first four nucleotides at the 5′ terminus and three PS bonds linking the last four nucleotides at the 3′ terminus.

In some embodiments, an sgRNA is provided comprising 2′-O-Me modified nucleotides at the first three nucleotides at the 5′ terminus; 2′-O-Me modified nucleotides at LS1, LS6, LS7, LS8, LS11, and LS12; 2′-F modified nucleotides at LS9 and LS10; 2′-O-Me modified nucleotides at US1-US12; 2′-O-Me modified nucleotides at H1-1-H1-12; a 2′-O-Me modified nucleotide between Hairpin 1 and Hairpin 2; 2′-O-Me modified nucleotides at H2-1-H2-15; and 2′-O-Me modified nucleotides at the last four nucleotides at the 3′ terminus. In some embodiments, the sgRNA further comprises three phosphorothioate (PS) bonds linking the first four nucleotides at the 5′ terminus and three PS bonds linking the last four nucleotides at the 3′ terminus.

In some embodiments, an sgRNA is provided comprising 2′-O-Me modified nucleotides at the first three nucleotides at the 5′ terminus; 2′-O-Me modified nucleotides at US1-US12; 2′-O-Me modified nucleotides at H1-1-H1-12; a 2′-O-Me modified nucleotide between Hairpin 1 and Hairpin 2; 2′-O-Me modified nucleotides at H2-1-H2-8; 2′-F modified nucleotides at H2-9-H2-15; 2′-F modified nucleotides at the second from last, third from last, and fourth from last nucleotide at the 3′ terminus; and a 2′-O-Me modified nucleotide at the last nucleotide at the 3′ terminus. In some embodiments, the sgRNA further comprises three phosphorothioate (PS) bonds linking the first four nucleotides at the 5′ terminus and three PS bonds linking the last four nucleotides at the 3′ terminus.

In some embodiments, an sgRNA is provided comprising 2′-O-Me modified nucleotides at the first three nucleotides at the 5′ terminus; 2′-O-Me modified nucleotides at US1-US12; 2′-O-Me modified nucleotides at H1-2, H1-4, H1-6, H1-8, H1-10, and H1-12; 2′-F modified nucleotides at H1-1, H1-3, H1-5, H1-7, H1-9, and H1-11; a 2′-F modified nucleotide between Hairpin 1 and Hairpin 2; 2′-F modified nucleotides at H2-2, H2-4, H2-6, H2-8, H2-10, H2-12; and H2-14; 2′-O-Me modified nucleotides at H2-1, H2-3, H2-5, H2-7, H2-9, H2-11; H2-13, and H2-15; 2′-F modified nucleotides at the second from last, and fourth from last nucleotide at the 3′ terminus; and 2′-O-Me modified nucleotide at the third from last, and last nucleotide at the 3′ terminus. In some embodiments, the sgRNA further comprises three phosphorothioate (PS) bonds linking the first four nucleotides at the 5′ terminus and three PS bonds linking the last four nucleotides at the 3′ terminus.

Disclosed herein, in some embodiments, is an sgRNA comprising 2′-O-Me modifications at nucleotides LS8, LS10, LS12, H1-2, H1-4, H1-6, H1-8, H1-10, H1-12, H2-1, H2-3, H2-5, H2-7, H2-9, H2-11, H2-13, and H2-15; and 2′-F modifications at LS7, LS9, LS11; H1-1, H1-3, H1-5, H1-7, H1-9, H1-11, H1-13, H2-2, H2-4, H2-6, H2-8, H2-10, H2-12, and H2-14. In some embodiments, the sgRNA further comprises three phosphorothioate (PS) bonds linking the first four nucleotides at the 5′ terminus and three PS bonds linking the last four nucleotides at the 3′ terminus. In some embodiments, the sgRNA further comprises 2′-O-Me modified nucleotides at the last and third to last nucleotide at the 3′ terminus; and 2′-F modified nucleotides at the second to last and third to last nucleotide at the 3′ terminus.

In some embodiments, an sgRNA comprising a 5′ end modification and one or more modifications in one or more of: the upper stem region; the hairpin 1 region; and the hairpin 2 region is provided, wherein the 5′ end modification comprises at least two phosphorothioate linkages within the first seven nucleotides of the 5′ terminus.

In some embodiments, an sgRNA comprising a 5′ end modification and one or more modifications in one or more of: the upper stem region; the hairpin 1 region; and the hairpin 2 region is provided, wherein the 5′ end modification comprises one or more phosphorothioate linkages at the 5′ end. In some embodiments, one or more phosphorothioate bonds link the 5′ terminal nucleotides.

In some embodiments, an sgRNA is provided, comprising a 5′ end modification and one or more modifications in one or more of: the upper stem region; the hairpin 1 region; and the hairpin 2 region is provided, wherein the 5′ end modification comprises one or more phosphorothioate linkages within the first seven nucleotides of the 5′ terminus.

In some embodiments, an sgRNA comprising any one of the modified sequences of SEQ ID Nos: 601 or 607-732 is provided, wherein the sgRNA further comprises a 5′ guide region that is at least partially complementary to a target sequence, and optionally directs a Cas9 to its target for cleavage.

In some embodiments, an sgRNA comprising nucleotides having at least 99, 98, 97, 96, 95, 94, 93, 92, 91, 90, 85, 80, 75, or 70% identity to the nucleotides of any one of SEQ ID Nos: 401-532, 601, 607-732, 801, 807-932, 1001, or 1007-1132, is provided, wherein the modification pattern is identical to the modification pattern shown in the reference sequence identifier. That is, the nucleotides A, U (and/or T in the case of deoxyribonucleotide modifications), C, and G may differ by 99, 98, 97, 96, 95, 94, 93, 92, 91, 90, 85, 80, 75, or 70% compared to what is shown in in the sequences, but the modification remains unchanged.

In some embodiments, an sgRNA is provided comprising 2′-O-Me modified nucleotides at: the first three nucleotides in the 5′ terminus; LS1, LS6, LS7, LS8, LS11, and LS12 in the lower stem; B1 and B2 in the bulge region; each of the nucleotides in the upper stem region; N16, N17, and N18 in the nexus region; each of the nucleotides in the hairpin 1 region; one nucleotide between hairpin 1 and hairpin 2; each of the nucleotides in the hairpin 2 region; and the last four nucleotides at the 3′ terminus. In some embodiments, the sgRNA further comprises three PS bonds between the first four nucleotides at the 5′ terminus and three PS bonds between the last four nucleotides at the 3′ terminus.

In some embodiments, an sgRNA is provided comprising 2′-O-Me modified nucleotides at: the first three nucleotides in the 5′ terminus; LS1, LS6, LS7, LS8, LS11, and LS12 in the lower stem; B1-B6 in the bulge region; each of the nucleotides in the upper stem region; N16, N17, and N18 in the nexus region; each of the nucleotides in the hairpin 1 region; one nucleotide between hairpin 1 and hairpin 2; each of the nucleotides in the hairpin 2 region; and the last four nucleotides at the 3′ terminus. In some embodiments, the sgRNA further comprises three PS bonds between the first four nucleotides at the 5′ terminus and three PS bonds between the last four nucleotides in the 3′ terminus region.

In some embodiments, an sgRNA is provided comprising 2′-F modified nucleotides at: LS9 and LS10 in the lower stem; 15-N18 in the nexus region; H2-9-HS-15 in the hairpin 2 region; and the second to last, third to last, and fourth to last nucleotide in the 3′ terminus region.

In some embodiments, an sgRNA is provided comprising 2′-F modified nucleotides at: each nucleotide in the lower stem; 15-N18 in the nexus region; H2-9-HS-15 in the hairpin 2 region; and the second to last, third to last, and fourth to last nucleotide in the 3′ terminus region.

In some embodiments, an sgRNA is provided comprising 2′-O-Me modified nucleotides at LS8, LS10, LS12, H1-2, H1-4, H1-6, H1-8, H1-10, H1-12, H2-1, H2-3, H2-5, H2-7, H2-9, H2-11, H2-13, H2-15, and the last and third to last nucleotides in the 3′ terminus region; and 2′-F modifications at LS7, LS9, LS11; H1-1, H1-3, H1-5, H1-7, H1-9, H1-11, H1-13, H2-2, H2-4, H2-6, H2-8, H2-10, H2-12, H2-14, and the second to last and fourth to last nucleotide in the 3′ terminus region.

In some embodiments, a single guide RNA (sgRNA) comprises one or more guide region YA site modifications or conserved region YA modifications, a 5′ end modification and one or more modification in one or more of: the upper stem region; the hairpin 1 region; and the hairpin 2 region, wherein the 5′ end modification comprises at least two phosphorothioate linkages within the first seven nucleotides at the 5′ end of the 5′ terminus. In some instances, the modification is a 2′-O-methyl (2′-O-Me) modified nucleotide. In some embodiments, the modification is a 2′-fluoro (2′-F) modified nucleotide.

In some embodiments, the sgRNA comprises one or more guide region YA site modifications or conserved region YA modifications, modifications at US1 to US12 and/or a modification at H1-1 and/or a modification in H2-1. In some embodiments, the sgRNA comprises one or more guide region YA site modifications or conserved region YA modifications and modifications at H1-1 to H1-12 and/or H2-1 to H2-15. In some embodiments, the sgRNA comprises one or more guide region YA site modifications or conserved region YA modifications and one or more modifications in each of the upper stem region, the hairpin 1 region, and the hairpin 2 region. In some embodiments, the sgRNA comprises one or more guide region YA site modifications or conserved region YA modifications and a modified nucleotide between hairpin 1 and hairpin 2 regions. In some embodiments, the sgRNA comprises one or more guide region YA site modifications or conserved region YA modifications and a modification in the lower stem region.

In some embodiments, the sgRNA comprises one or more guide region YA site modifications or conserved region YA modifications and a modification in the bulge region. In some embodiments, 50% of the nucleotides in the bulge region are modified, wherein the modification is 2′-O-Me or 2′-F.

In some embodiments, the sgRNA comprises one or more guide region YA site modifications or conserved region YA modifications and a modification in the nexus region. In some embodiments, the sgRNA comprises modifications at N15, N16, N17, and/or N18 in the nexus region, wherein the modification is 2′-O-Me or 2′-F. In some instances, N16, N17, and N18 are linked with PS bonds.

In some embodiments, the sgRNA comprises one or more guide region YA site modifications or conserved region YA modifications and modifications at the first four nucleotides at the 5′ end of the 5′ terminus and the last four nucleotides at the 3′ end of the 3′terminus. In some instances, these modifications are linking PS bond (i.e., PS bonds that link the first four and last four nucleotides). In some embodiments, the sgRNA further comprises 2′-O-Me modifications at the first three nucleotides at the 5′ end of the 5′ terminus and the last three nucleotides at the 3′ end of the 3′ terminus.

In some embodiments, the sgRNA comprises one or more guide region YA site modifications or conserved region YA modifications and modifications LS1, LS6, LS7, LS8, LS11, and LS12, wherein the modification is 2′-O-Me or 2′-F.

In some embodiments, the sgRNA comprises one or more guide region YA site modifications or conserved region YA modifications and modifications at each of the nucleotides in the bulge region, wherein the modification is 2′-O-Me or 2′-F.

In some embodiments, the sgRNA comprises one or more guide region YA site modifications or conserved region YA modifications and modifications at each of the nucleotides in the upper stem region, wherein the modification is 2′-O-Me or 2′-F.

In some embodiments, the sgRNA comprises one or more guide region YA site modifications or conserved region YA modifications and modifications at each of the nucleotides in the hairpin 1 region, wherein the modification is 2′-O-Me or 2′-F.

In some embodiments, the sgRNA comprises one or more guide region YA site modifications or conserved region YA modifications and modifications at each of the nucleotides in the hairpin 2 region, wherein the modification is 2′-O-Me or 2′-F.

In some embodiments, an sgRNA is encompassed comprising one or more guide region YA site modifications or conserved region YA modifications and further comprising 2′-O-Me modified nucleotides at the following positions:
a. LS1, LS6, LS7, LS8, LS11, and/or LS12 in the lower stem region;
b. B1 and/or B2 in the bulge region;
c. each nucleotide in the upper stem region;
d. N16, N17, and/or N18 in the nexus region;
e. each nucleotide in the hairpin 1 region; and
f. each nucleotide in the hairpin 2 region.
   In some embodiments, B3-B6 are modified with 2′-O-Me. In some instances, the sgRNA further comprising a 5′ protective end modification, a 3′ protective end modification, or both a 3′ and a 5′ protective end modification. In some embodiments, the sgRNA comprises 2′-F modifications at LS9 and LS10. In some embodiments, the sgRNA comprises 2′F modifications at N15, N16, N17, and N18. In some embodiments, the sgRNA comprises 2′F modifications at H2-9, H2-10, H2-11, H2-12, H2-13, H2-14, and H2-15. In some embodiments, the sgRNA comprises 2′F modifications at the second to last, third to last, and fourth to last nucleotides at the 3′ end of the 3′ terminus.

In some embodiments, an sgRNA is encompassed comprising one or more guide region YA site modifications or conserved region YA modifications and 2′-F modified nucleotides at the following positions:
a. LS9 and LS10 in the lower stem region;
b. N15, N16, N17, and N18 in the nexus region; and
c. H2-9, H2-10, H2-11, H2-12, H2-13, H2-14, and H2-15 in the hairpin 2 region.
   In some embodiments, the sgRNA comprises 2′-F modified nucleotides at the second to last, third to last, and fourth to last nucleotides at the 3′ terminus. In some embodiments, the sgRNA comprises three phosphorothioate (PS) bonds linking the first four nucleotides at the 5′ end of the 5′ terminus and three PS bonds linking the last four nucleotides at the 3′ end of the 3′ terminus. In some embodiments, the sgRNA comprises 2′-O-Me or 2′-F modified nucleotides at the first three nucleotides at the 5′ end of the 5′ terminus, and 2′-O-Me or 2′-F modified nucleotides at three of the last four nucleotides at the 3′ end of the 3′ terminus.

In some embodiments, an sgRNA is encompassed comprising one or more guide region YA site modifications or conserved region YA modifications and further comprising:
a. 2′-O-Me modified nucleotides at the first three nucleotides at the 5′ end of the 5′ terminus;
b. Optional 2′-O-Me modified nucleotides at LS1 and/or LS6;
c. 2′-O-Me modified nucleotides at US1-US12;
d. 2′-O-Me modified nucleotides at H1-1-H1-12;
e. Optional 2′-O-Me modified nucleotide between Hairpin 1 and Hairpin 2;
f. 2′-O-Me modified nucleotides at H2-1-H2-15; and
g. 2′-O-Me modified nucleotides at the last four nucleotides at the 3′ end of the 3′ terminus; and optionally a 5′ protective end modification, a 3′ protective end modification, or both a 3′ and a 5′ protective end modification.

In some embodiments, an sgRNA is encompassed comprising one or more guide region YA site modifications or conserved region YA modifications and:
a. 2′-O-Me modified nucleotides at the first three nucleotides at the 5′ end of the 5′ terminus;
b. 2′-F modified nucleotides at LS1-LS6;
c. 2′-O-Me modified nucleotides at US1-US12;
d. 2′-O-Me modified nucleotides at H1-1-H1-12;
e. a 2′-O-Me modified nucleotide between Hairpin 1 and Hairpin 2;
f. 2′-O-Me modified nucleotides at H2-1-H2-15; and
g. 2′-O-Me modified nucleotides at the last four nucleotides at the 3′ end of the 3′ terminus; and optionally a 5′ protective end modification, a 3′ protective end modification, or both a 3′ and a 5′ protective end modification.

In some embodiments, an sgRNA is encompassed comprising one or more guide region YA site modifications or conserved region YA modifications and further comprising:
a. 2′-O-Me modified nucleotides at the first three nucleotides at the 5′ terminus;
b. 2′-F modified nucleotides at LS2-LSS;
c. 2′-O-Me modified nucleotides at LS1 and LS6;
d. 2′-O-Me modified nucleotides at US1-US12;
e. 2′-O-Me modified nucleotides at H1-1-H1-12;
f. a 2′-O-Me modified nucleotide between Hairpin 1 and Hairpin 2;
g. 2′-O-Me modified nucleotides at H2-1-H2-15; and
h. 2′-O-Me modified nucleotides at the last four nucleotides at the 3′ terminus and optionally a 5′ protective end modification, a 3′ protective end modification, or both a 3′ and a 5′ protective end modification.

In some embodiments, a sgRNA is encompassed comprising one or more guide region YA site modifications or conserved region YA modifications and further comprising:
a. 2′-O-Me modified nucleotides at the first three nucleotides at the 5′ terminus;
b. 2′-O-Me modified nucleotides at US1-US12;
c. 2′-O-Me modified nucleotides at LS7, LS8, LS11, and LS12;
d. 2′-O-Me modified nucleotides at H1-1-H1-12;
e. a 2′-O-Me modified nucleotide between Hairpin 1 and Hairpin 2;
f. 2′-O-Me modified nucleotides at H2-1-H2-15; and
g. 2′-O-Me modified nucleotides at the last four nucleotides at the 3′ terminus,
   and optionally a 5′ protective end modification, a 3′ protective end modification, or both a 3′ and a 5′ protective end modification.

In some embodiments, a sgRNA is encompassed comprising one or more guide region YA site modifications or conserved region YA modifications and further comprising:
a. 2′-O-Me modified nucleotides at the first three nucleotides at the 5′ terminus;
b. 2′-O-Me modified nucleotides at US1-US12;
c. 2′-O-Me modified nucleotides at LS7, LS8, LS11, and LS12;
d. 2′-F modified nucleotides at LS9 and LS10;
e. 2′-O-Me modified nucleotides at H1-1-H1-12;
f. a 2′-O-Me modified nucleotide between Hairpin 1 and Hairpin 2;
g. 2′-O-Me modified nucleotides at H2-1-H2-15; and
h. 2′-O-Me modified nucleotides at the last four nucleotides at the 3′ terminus,
   and optionally a 5′ protective end modification, a 3′ protective end modification, or both a 3′ and a 5′ protective end modification.

In some embodiments, an sgRNA is encompassed comprising one or more guide region YA site modifications or conserved region YA modifications and further comprising:
a. 2′-O-Me modified nucleotides at the first three nucleotides at the 5′ terminus;
b. 2′-O-Me modified nucleotides at US1-US12;
c. 2′-O-Me modified nucleotides at LS8, LS10, and LS12;
d. 2′-O-F modified nucleotides at LS7, LS9, and LS11;
e. 2′-O-Me modified nucleotides at H1-1-H1-12;
f. a 2′-O-Me modified nucleotide between Hairpin 1 and Hairpin 2;
g. 2′-O-Me modified nucleotides at H2-1-H2-15; and
h. 2′-O-Me modified nucleotides at the last four nucleotides at the 3′ terminus, and optionally a 5′ protective end modification, a 3′ protective end modification, or both a 3′ and a 5′ protective end modification.

In some embodiments, a sgRNA is encompassed comprising one or more guide region YA site modifications or conserved region YA modifications and further comprising:
a. 2′-O-Me modified nucleotides at the first three nucleotides at the 5′ terminus;
b. 2′-O-Me modified nucleotides at LS1, LS6, LS7, LS8, LS11, and LS12
c. 2′-O-Me modified nucleotides at US1-US12;
d. 2′-O-Me modified nucleotides at H1-1-H1-12;
e. a 2′-O-Me modified nucleotide between Hairpin 1 and Hairpin 2;
f. 2′-O-Me modified nucleotides at H2-1-H2-15; and
g. 2′-O-Me modified nucleotides at the last four nucleotides at the 3′ terminus, and optionally a 5′ protective end modification, a 3′ protective end modification, or both a 3′ and a 5′ protective end modification.

In some embodiments, a sgRNA is encompassed comprising one or more guide region YA site modifications or conserved region YA modifications and further comprising:
a. 2′-O-Me modified nucleotides at the first three nucleotides at the 5′ terminus;
b. 2′-O-Me modified nucleotides at LS1, LS6, LS7, LS8, LS11, and LS12;
c. 2′-F modified nucleotides at LS9 and LS10;
d. 2′-O-Me modified nucleotides at US1-US12;
e. 2′-O-Me modified nucleotides at H1-1-H1-12;
f. a 2′-O-Me modified nucleotide between Hairpin 1 and Hairpin 2;
g. 2′-O-Me modified nucleotides at H2-1-H2-15; and
h. 2′-O-Me modified nucleotides at the last four nucleotides at the 3′ terminus, and optionally a 5′ protective end modification, a 3′ protective end modification, or both a 3′ and a 5′ protective end modification.

In some embodiments, a sgRNA is encompassed comprising one or more guide region YA site modifications or conserved region YA modifications and further comprising:
a. 2′-O-Me modified nucleotides at the first three nucleotides at the 5′ end of the 5′ terminus;
b. 2′-O-Me modified nucleotides at US1-US12;
c. 2′-O-Me modified nucleotides at H1-1-H1-12;
d. a 2′-O-Me modified nucleotide between Hairpin 1 and Hairpin 2;
e. 2′-O-Me modified nucleotides at H2-1-H2-8;
f. 2′-F modified nucleotides at H2-9-H2-15;
g. 2′-F modified nucleotides at the second from last, third from last, and fourth from last nucleotide at the 3′ terminus; and
h. a 2′-O-Me modified nucleotide at the last nucleotide at the 3′ terminus, and optionally a 5′ protective end modification, a 3′ protective end modification, or both a 3′ and a 5′ protective end modification.

In some embodiments, a sgRNA is encompassed comprising one or more guide region YA site modifications or conserved region YA modifications and further comprising:
a. 2′-O-Me modified nucleotides at the first three nucleotides at the 5′ end of the 5′ terminus;
b. 2′-O-Me modified nucleotides at US1-US12;
c. 2′-O-Me modified nucleotides at H1-2, H1-4, H1-6, H1-8, H1-10, and H1-12;
d. 2′-F modified nucleotides at H1-1, H1-3, H1-5, H1-7, H1-9, and H1-11;
e. a 2′-F modified nucleotide between Hairpin 1 and Hairpin 2;
f. 2′-F modified nucleotides at H2-2, H2-4, H2-6, H2-8, H2-10, H2-12; and H2-14;
g. 2′-O-Me modified nucleotides at H2-1, H2-3, H2-5, H2-7, H2-9, H2-11; H2-13, and H2-15;
h. 2′-F modified nucleotides at the second from last, and fourth from last nucleotide at the 3′ terminus; and
i. 2′-O-Me modified nucleotide at the third from last and last nucleotide at the 3′ end of the 3′ terminus,
   and optionally a 5′ protective end modification, a 3′ protective end modification, or both a 3′ and a 5′ protective end modification.

In some embodiments, a sgRNA is encompassed comprising one or more guide region YA site modifications or conserved region YA modifications and further comprising:
a. 2′-O-Me modified nucleotides LS8, LS10, LS12, H1-2, H1-4, H1-6, H1-8, H1-10, H1-12, H2-1, H2-3, H2-5, H2-7, H2-9, H2-11, H2-13, and H2-15; and
b. 2′-F modified nucleotides at LS7, LS9, LS11; H1-1, H1-3, H1-5, H1-7, H1-9, H1-11, H1-13, H2-2, H2-4, H2-6, H2-8, H2-10, H2-12, and H2-14, and optionally further comprising three phosphorothioate (PS) bonds linking the first four nucleotides at the 5′ end of the 5′ terminus and three PS bonds linking the last four nucleotides at the 3′ end of the 3′ terminus; and optionally further comprising:
c. 2′-O-Me modified nucleotides at the last and third to last nucleotide at the 3′ end of the 3′ terminus; and/or
d. 2′-F modified nucleotides at the second to last, fourth to last, and/or last nucleotide at the 3′ end of the 3′ terminus.

Any of the foregoing modification patterns can be combined with a modification pattern set forth in the embodiments described above, e.g., in the summary section or Table 1, to the extent that they are non-overlapping. In the event that combining a foregoing modification pattern with a modification pattern set forth in the summary section or Table 1 would result in incompatible modifications (e.g., the same position would be both 2′-OMe and 2′-fluoro), the modification set forth in the summary section or Table 1 controls.

### Short-Single Guide RNA (Short-sgRNA)

In some embodiments, an sgRNA provided herein is a short-single guide RNAs (short-sgRNAs), e.g., comprising a conserved portion of an sgRNA comprising a hairpin region, wherein the hairpin region lacks at least 5-10 nucleotides or 6-10 nucleotides. In some embodiments, the sgRNA is from S. pyogenes Cas9 (“spyCas9”) or a spyCas9 equivalent. In some embodiments, the sgRNA is not from S. pyogenes Cas9 (“non-spyCas9”). In some embodiments, the 5-10 nucleotides or 6-10 nucleotides are consecutive.

In some embodiments, a short-sgRNA lacks at least nucleotides 54-58 (AAAAA) of the conserved portion of a spyCas9 sgRNA, as shown in Table 2. In some embodiments, a short-sgRNA is a non-spyCas9 sgRNA that lacks nucleotides corresponding to nucleotides 54-58 (AAAAA) of the conserved portion of a spyCas9 as determined, for example, by pairwise or structural alignment. In some embodiments, the non-spyCas9 sgRNA is Staphylococcus aureus Cas9 (“saCas9”) sgRNA.

In some embodiments, the hairpin regions lacks 5, 6, 7, 8, 9, 10, 11, or 12 nucleotides. In some embodiments, the hairpin 1 portion lacks 5, 6, 7, 8, 9, 10, 11, or 12 nucleotides. In some embodiments, the hairpin 2 portion lacks 5, 6, 7, 8, 9, 10, 11, or 12 nucleotides. In some embodiments, the hairpin regions lacks 5, 6, 7, 8, 9, 10, 11, or 12 consecutive nucleotides. In some embodiments, the hairpin 1 portion lacks 5, 6, 7, 8, 9, 10, 11, or 12 consecutive nucleotides. In some embodiments, the hairpin 2 portion lacks 5, 6, 7, 8, 9, 10, 11, or 12 consecutive nucleotides. In some embodiments, the 5-10 lacking nucleotides or 6-10 lacking nucleotides are within hairpin 1. In some embodiments, the 5-10 lacking nucleotides or 6-10 lacking nucleotides are within hairpin 2. In some embodiments, the 5-10 lacking nucleotides or 6-10 lacking nucleotides are within hairpin 1 and hairpin 2. In some embodiments, the 5-10 lacking nucleotides or 6-10 lacking nucleotides are within hairpin 1 or hairpin 2. In some embodiments, the 5-10 lacking nucleotides or 6-10 lacking nucleotides are consecutive and include the “N” between hairpin 1 and hairpin 2. In some embodiments, the 5-10 or 6-10 lacking nucleotides include the “N” between hairpin 1 and hairpin 2. In some embodiments, the 5-10 or 6-10 lacking nucleotides are consecutive and span at least a portion of hairpin 1. In some embodiments, the 5-10 or 6-10 lacking nucleotides are consecutive and span at least a portion of hairpin 2. In some embodiments, the 5-10 lacking nucleotides or 6-10 lacking nucleotides are consecutive and span at least a portion of hairpin 1 and a portion of hairpin 2. In some embodiments, the 5-10 lacking nucleotides or 6-10 lacking nucleotides are consecutive and span at least a portion of hairpin 1 and the “N” between hairpin 1 and hairpin 2. In some embodiments, the 5-10 lacking nucleotides comprise or consist of nucleotides 54-58, 54-61, or 53-60 of SEQ ID NO: 400.

In some embodiments, the short-sgRNA described herein further comprises a nexus region, wherein the nexus region lacks at least one nucleotide. In some embodiments, the short-sgRNA lacks at least 2, 3, 4, 5, 6, 7, 8, 9, or 10 nucleotides in the nexus region. In some embodiments, the short-sgRNA lacks at least 1-2, 1-3, 1-4 nucleotides, 1-5 nucleotides, 1-6 nucleotides, 1-10 nucleotides, or 1-15 nucleotides in the nexus region. In some embodiments, the short-sgRNA lacks each nucleotide in the nexus region.

In some embodiments, the short-sgRNA further comprises a guide region. In some embodiments, the guide region comprises the first 1-10, 11, 12, 13, 14, 15, 16, 17, 18, 19, or 20 nucleotides at the 5′ end of the short-sgRNA. In some embodiments, the guide region comprises 20 nucleotides. In some embodiments, the guide region comprises 5, 6, 7, 8, 9, 10, 11, 12, 13, 14, 15, 16, 17, 18, 19, 20, 21, 22, 23, 24, or 25 or more nucleotides. In some embodiments, the guide region comprises 17 nucleotides. In some embodiments, the guide region comprises 18 nucleotides. In some embodiments, the guide region comprises 19 nucleotides.

In some embodiments, the selection of the guide region is determined based on target sequences within the gene of interest for editing. For example, in some embodiments, the short-sgRNA comprises a guide region that is complementary to target sequences of a gene of interest.

In some embodiments, the target sequence in the gene of interest may be complementary to the guide region of the short-sgRNA. In some embodiments, the degree of complementarity or identity between a guide region of a short-sgRNA and its corresponding target sequence in the gene of interest may be about 50%, 55%, 60%, 65%, 70%, 75%, 80%, 85%, 90%, 95%, 96%, 97%, 98%, 99%, or 100%. In some embodiments, the guide region of a short-sgRNA and the target region of a gene of interest may be 100% complementary or identical. In other embodiments, the guide region of a short-sgRNA and the target region of a gene of interest may contain at least one mismatch. For example, the guide region of a short-sgRNA and the target sequence of a gene of interest may contain 1, 2, 3, 4, 5, 6, 7, 8, 9, or 10 mismatches, where the total length of the target sequence is at least about 17, 18, 19, 20 or more base pairs. In some embodiments, the guide region of a short-sgRNA and the target region of a gene of interest may contain 1-6 mismatches where the guide sequence comprises at least about 17, 18, 19, 20 or more nucleotides. In some embodiments, the guide region of a short-sgRNA and the target region of a gene of interest may contain 1, 2, 3, 4, 5, or 6 mismatches where the guide sequence comprises about 20 nucleotides. The 5′ terminus may comprise nucleotides that are not considered guide regions (i.e., do not function to direct a Cas9 protein to a target nucleic acid).

### Modified Short-Single Guide RNA (Short-sgRNA)

In some embodiments, the short-sgRNA is modified. The term “modified” or “modification” in the context of a short-sgRNA described herein includes, the modifications described above, including, for example, (a) end modifications, e.g., 5′ end modifications or 3′ end modifications, including 5′ or 3′ protective end modifications, (b) nucleobase (or “base”) modifications, including replacement or removal of bases, (c) sugar modifications, including modifications at the 2′, 3′, and/or 4′ positions, (d) internucleoside linkage modifications, and (e) backbone modifications, which can include modification or replacement of the phosphodiester linkages and/or the ribose sugar. A modification of a nucleotide at a given position includes a modification or replacement of the phosphodiester linkage immediately 3′ of the sugar of the nucleotide. Thus, for example, a nucleic acid comprising a phosphorothioate between the first and second sugars from the 5′ end is considered to comprise a modification at position 1. The term “modified short-sgRNA” generally refers to a short-sgRNA having a modification to the chemical structure of one or more of the base, the sugar, and the phosphodiester linkage or backbone portions, including nucleotide phosphates, all as detailed and exemplified herein.

Exemplary patterns of modifications are shown in Table 1. Additional exemplary patterns are discussed below.

Modifications of Guide Regions and/or YA Sites

In some embodiments, a short-sgRNA comprises modifications at 1, 2, 3, 4, 5, 6, 7, 8, 9, 10, 11, 12, 13, 14, 15, 16, or more YA sites. In some embodiments, the pyrimidine of the YA site comprises a modification (which includes a modification altering the internucleoside linkage immediately 3′ of the sugar of the pyrimidine). In some embodiments, the adenine of the YA site comprises a modification (which includes a modification altering the internucleoside linkage immediately 3′ of the sugar of the adenine). In some embodiments, the pyrimidine and the adenine of the YA site comprise modifications, such as sugar, base, or internucleoside linkage modifications. The YA modifications can be any of the types of modifications set forth herein. In some embodiments, the YA modifications comprise one or more of phosphorothioate, 2′-OMe, or 2′-fluoro. In some embodiments, the YA modifications comprise pyrimidine modifications comprising one or more of phosphorothioate, 2′-OMe, 2′-H, inosine, or 2′-fluoro. In some embodiments, the YA modification comprises a bicyclic ribose analog (e.g., an LNA, BNA, or ENA) within an RNA duplex region that contains one or more YA sites. In some embodiments, the YA modification comprises a bicyclic ribose analog (e.g., an LNA, BNA, or ENA) within an RNA duplex region that contains a YA site, wherein the YA modification is distal to the YA site.

Guide Region Modifications, including YA Site Modifications

In some embodiments, the guide region comprises 1, 2, 3, 4, 5, or more YA sites (“guide region YA sites”) that may comprise YA modifications. In some embodiments, one or more YA sites located at 5-end, 6-end, 7-end, 8-end, 9-end, or 10-end from the 5′ end of the 5′ terminus (where “5-end”, etc., refers to position 5 to the 3′ end of the guide region, i.e., the most 3′ nucleotide in the guide region) comprise YA modifications. In some embodiments, two or more YA sites located at 5-end, 6-end, 7-end, 8-end, 9-end, or 10-end from the 5′ end of the 5′ terminus comprise YA modifications. In some embodiments, three or more YA sites located at 5-end, 6-end, 7-end, 8-end, 9-end, or 10-end from the 5′ end of the 5′ terminus comprise YA modifications. In some embodiments, four or more YA sites located at 5-end, 6-end, 7-end, 8-end, 9-end, or 10-end from the 5′ end of the 5′ terminus comprise YA modifications. In some embodiments, five or more YA sites located at 5-end, 6-end, 7-end, 8-end, 9-end, or 10-end from the 5′ end of the 5′ terminus comprise YA modifications. A modified guide region YA site comprises a YA modification.

In some embodiments, a modified guide region YA site is within 17, 16, 15, 14, 13, 12, 11, 10, or 9 nucleotides of the 3′ terminal nucleotide of the guide region. For example, if a modified guide region YA site is within 10 nucleotides of the 3′ terminal nucleotide of the guide region and the guide region is 20 nucleotides long, then the modified nucleotide of the modified guide region YA site is located at any of positions 11-20. In some embodiments, a YA modification is located within a YA site 20, 19, 18, 17, 16, 15, 14, 13, 12, 11, 10, 9, 8, 7, 6, 5, 4, 3, 2, or 1 nucleotides from the 3′ terminal nucleotide of the guide region. In some embodiments, a YA modification is located 20, 19, 18, 17, 16, 15, 14, 13, 12, 11, 10, 9, 8, 7, 6, 5, 4, 3, 2, or 1 nucleotides from the 3′ terminal nucleotide of the guide region.

In some embodiments, a modified guide region YA site is at or after nucleotide 4, 5, 6, 7, 8, 9, 10, or 11 from the 5′ end of the 5′ terminus.

In some embodiments, a modified guide region YA site is other than a 5′ end modification. For example, a short-sgRNA can comprise a 5′ end modification as described herein and further comprise a modified guide region YA site. Alternatively, a short-sgRNA can comprise an unmodified 5′ end and a modified guide region YA site. Alternatively, a short-sgRNA can comprise a modified 5′ end and an unmodified guide region YA site.

In some embodiments, a modified guide region YA site comprises a modification that at least one nucleotide located 5′ of the guide region YA site does not comprise. For example, if nucleotides 1-3 comprise phosphorothioates, nucleotide 4 comprises only a 2′-OMe modification, and nucleotide 5 is the pyrimidine of a YA site and comprises a phosphorothioate, then the modified guide region YA site comprises a modification (phosphorothioate) that at least one nucleotide located 5′ of the guide region YA site (nucleotide 4) does not comprise. In another example, if nucleotides 1-3 comprise phosphorothioates, and nucleotide 4 is the pyrimidine of a YA site and comprises a 2′-OMe, then the modified guide region YA site comprises a modification (2′-OMe) that at least one nucleotide located 5′ of the guide region YA site (any of nucleotides 1-3) does not comprise. This condition is also always satisfied if an unmodified nucleotide is located 5′ of the modified guide region YA site.

In some embodiments, the modified guide region YA sites comprise modifications as described for YA sites above.

Additional embodiments of guide region modifications, including guide region YA site modifications, are set forth elsewhere herein, including in the summary above and in the discussion of gRNAs comprising modifications, including modifications at YA sites above, and elsewhere herein. The guide region of a short-sgRNA may be modified according to any embodiment comprising a modified guide region set forth herein. Any embodiments set forth elsewhere in this disclosure may be combined to the extent feasible with any of the foregoing embodiments.

Conserved Region YA Site Modifications

Conserved region YA sites 1-10 are illustrated in FIG. 1B. In some embodiments, 1, 2, 3, 4, 5, 6, 7, 8, 9, or 10 conserved region YA sites comprise modifications.

In some embodiments, conserved region YA sites 1, 8, or 1 and 8 comprise YA modifications. In some embodiments, conserved region YA sites 1, 2, 3, 4, and 10 comprise YA modifications. In some embodiments, YA sites 2, 3, 4, 8, and 10 comprise YA modifications. In some embodiments, conserved region YA sites 1, 2, 3, and 10 comprise YA modifications. In some embodiments, YA sites 2, 3, 8, and 10 comprise YA modifications. In some embodiments, YA sites 1, 2, 3, 4, 8, and 10 comprise YA modifications. In some embodiments, 1, 2, 3, 4, 5, 6, 7, or 8 additional conserved region YA sites comprise YA modifications.

In some embodiments, 1, 2, 3, or 4 of conserved region YA sites 2, 3, 4, and 10 comprise YA modifications. In some embodiments, 1, 2, 3, 4, 5, 6, 7, or 8 additional conserved region YA sites comprise YA modifications.

In some embodiments, the modified conserved region YA sites comprise modifications as described for YA sites above.

Additional embodiments of conserved region YA site modifications are set forth in the summary above. Any embodiments set forth elsewhere in this disclosure may be combined to the extent feasible with any of the foregoing embodiments.

Modifications to Terminal Nucleotides

In some embodiments, the 5′ and/or 3′ terminus regions of a short-sgRNA are modified.

3′ Terminus Region Modifications

In some embodiments, the terminal (i.e., last) 1, 2, 3, 4, 5, 6, or 7 nucleotides in the 3′ terminus region are modified. Throughout, this modification may be referred to as a “3′ end modification”. In some embodiments, the terminal (i.e., last) 1, 2, 3, 4, 5, 6, or 7 nucleotides in the 3′ terminus region comprise more than one modification. In some embodiments, at least one of the terminal (i.e., last) 1, 2, 3, 4, 5, 6, or 7 nucleotides in the 3′ terminus region are modified. In some embodiments, at least two of the terminal (i.e., last) 1, 2, 3, 4, 5, 6, or 7 nucleotides in the 3′ terminus region are modified. In some embodiments, at least three of the terminal (i.e., last) 1, 2, 3, 4, 5, 6, or 7 nucleotides in the 3′ terminus region are modified. In some embodiments, the modification comprises a PS linkage. In some embodiments, the modification to the 3′ terminus region is a 3′ protective end modification. In some embodiments, the 3′ end modification comprises a 3′ protective end modification.

In some embodiments, the 3′ end modification comprises a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.

In some embodiments, the 3′ end modification comprises or further comprises a 2′-O-methyl (2′-O-Me) modified nucleotide.

In some embodiments, the 3′ end modification comprises or further comprises a 2′-fluoro (2′-F) modified nucleotide.

In some embodiments, the 3′ end modification comprises or further comprises a phosphorothioate (PS) linkage between nucleotides.

In some embodiments, the 3′ end modification comprises or further comprises an inverted abasic modified nucleotide.

In some embodiments, the 3′ end modification comprises or further comprises a modification of any one or more of the last 7, 6, 5, 4, 3, 2, or 1 nucleotides. In some embodiments, the 3′ end modification comprises or further comprises one modified nucleotide. In some embodiments, the 3′ end modification comprises or further comprises two modified nucleotides. In some embodiments, the 3′ end modification comprises or further comprises three modified nucleotides. In some embodiments, the 3′ end modification comprises or further comprises four modified nucleotides. In some embodiments, the 3′ end modification comprises or further comprises five modified nucleotides. In some embodiments, the 3′ end modification comprises or further comprises six modified nucleotides. In some embodiments, the 3′ end modification comprises or further comprises seven modified nucleotides.

In some embodiments, the 3′ end modification comprises or further comprises a modification of between 1 and 7 or between 1 and 5 nucleotides.

In some embodiments, the 3′ end modification comprises or further comprises modifications of 1, 2, 3, 4, 5, 6, or 7 nucleotides at the 3′ end of the gRNA.

In some embodiments, the 3′ end modification comprises or further comprises modifications of about 1-3, 1-5, 1-6, or 1-7 nucleotides at the 3′ end of the gRNA.

In some embodiments, the 3′ end modification comprises or further comprises any one or more of the following: a phosphorothioate (PS) linkage between nucleotides, a 2′-O-Me modified nucleotide, a 2′-O-moe modified nucleotide, a 2′-F modified nucleotide, an inverted abasic modified nucleotide, and a combination thereof.

In some embodiments, the 3′ end modification comprises or further comprises 1, 2, 3, 4, 5, 6, or 7 PS linkages between nucleotides.

In some embodiments, the 3′ end modification comprises or further comprises at least one 2′-O-Me, 2′-O-moe, inverted abasic, or 2′-F modified nucleotide. In some embodiments, the 3′ end modification comprises or further comprises one PS linkage, wherein the linkage is between the last and second to last nucleotide. In some embodiments, the 3′ end modification comprises or further comprises two PS linkages between the last three nucleotides. In some embodiments, the 3′ end modification comprises or further comprises four PS linkages between the last four nucleotides.

In some embodiments, the 3′ end modification comprises or further comprises PS linkages between any one or more of the last four nucleotides. In some embodiments, the 3′ end modification comprises or further comprises PS linkages between any one or more of the last five nucleotides. In some embodiments, the 3′ end modification comprises or further comprises PS linkages between any one or more of the last 2, 3, 4, 5, 6, or 7 nucleotides.

In some embodiments, the 3′ end modification comprises or further comprises a modification of one or more of the last 1-7 nucleotides, wherein the modification is a PS linkage, inverted abasic nucleotide, 2′-OMe, 2′-O-moe, 2′-F, or combinations thereof.

In some embodiments, the 3′ end modification comprises or further comprises a modification to the last nucleotide with 2′-OMe, 2′-O-moe, 2′-F, or combinations thereof, and an optionally one or two PS linkages to the next nucleotide and/or the first nucleotide of the 3′ tail.

In some embodiments, the 3′ end modification comprises or further comprises a modification to the last and/or second to last nucleotide with 2′-OMe, 2′-O-moe, 2′-F, or combinations thereof, and optionally one or more PS linkages.

In some embodiments, the 3′ end modification comprises or further comprises a modification to the last, second to last, and/or third to last nucleotides with 2′-OMe, 2′-O-moe, 2′-F, or combinations thereof, and optionally one or more PS linkages.

In some embodiments, the 3′ end modification comprises or further comprises a modification to the last, second to last, third to last, and/or fourth to last nucleotides with 2′-OMe, 2′-O-moe, 2′-F, or combinations thereof, and optionally one or more PS linkages.

In some embodiments, the 3′ end modification comprises or further comprises a modification to the last, second to last, third to last, fourth to last, and/or fifth to last nucleotides with 2′-OMe, 2′-O-moe, 2′-F, or combinations thereof, and optionally one or more PS linkages.

In some embodiments, the gRNA comprising a 3′ end modification comprises or further comprises a 3′ tail, wherein the 3′ tail comprises a modification of any one or more of the nucleotides present in the 3′ tail. In some embodiments, the 3′ tail is fully modified. In some embodiments, the 3′ tail comprises 1, 2, 3, 4, 5, 6, 7, 8, 9, 10, 1-2, 1-3, 1-4, 1-5, 1-6, 1-7, 1-8, 1-9, or 1-10 nucleotides, optionally where any one or more of these nucleotides are modified.

In some embodiments, a gRNA is provided comprising a 3′ end modification, wherein the 3′ end modification comprises the 3′ end modification as shown in any one of SEQ ID Nos: 1-54. In some embodiments, a gRNA is provided comprising a 3′ protective end modification.

In some embodiments, agRNA is provided comprising a 3′ end modification, wherein the 3′ end modification comprises (i) a 2′-OMe modified nucleotide at the last nucleotide of the conserved region of an gRNA or short-sgRNA (ii) three consecutive 2′O-moe modified nucleotides immediately 5′ to the 2′-OMe modified nucleotide, and (iii) three consecutive PS linkages between the last three nucleotides.

In some embodiments, a gRNA is provided comprising a 3′ end modification, wherein the 3′ end modification comprises (i) five consecutive 2′-OMe modified nucleotides from the last nucleotide of the conserved region of an sgRNA or the conserved region of a short-sgRNA, and (ii) three PS linkages between the last three nucleotides.

In some embodiments, a gRNA is provided comprising a 3′ end modification, wherein the 3′ end modification comprises an inverted abasic modified nucleotide at the last nucleotide of the conserved region of an sgRNA or the conserved region of a short-sgRNA.

In some embodiments, a gRNA is provided comprising a 3′ end modification, wherein the 3′ end modification comprises (i) an inverted abasic modified nucleotide at the last nucleotide of the conserved region of an sgRNA or short-sgRNA, and (ii) three consecutive 2′-OMe modified nucleotides at the last three nucleotides of the conserved region of an sgRNA or the conserved region of a short-sgRNA.

In some embodiments, a gRNA is provided comprising (i) 15 consecutive 2′-OMe modified nucleotides from the last nucleotide of the conserved region of an sgRNA or short-sgRNA, (ii) five consecutive 2′-F modified nucleotides immediately 5′ to the 2′-OMe modified nucleotides, and (iii) three PS linkages between the last three nucleotides.

In some embodiments, a short-sgRNA is provided comprising (i) alternating 2′-OMe modified nucleotides and 2′-F modified nucleotides at the last 20 nucleotides of the conserved region of an sgRNA or short-sgRNA, and (ii) three PS linkages between the last three nucleotides.

In some embodiments, a short-sgRNA is provided comprising a 3′ end modification, wherein the 3′ end modification comprises (i) two or three consecutive 2′-OMe modified nucleotides, and (ii) three PS linkages between the last three nucleotides.

In some embodiments, a short-sgRNA is provided comprising a 3′ end modification, wherein the 3′ end modification comprises one PS linkage between the last and next to last nucleotides.

In some embodiments, a short-sgRNA is provided comprising (i) 15 or 20 consecutive 2′-OMe modified nucleotides, and (ii) three PS linkages between the last three nucleotides.

In some embodiments, the short-sgRNA comprises a 5′ end modification and a 3′ end modification.

### 3′ Tail

In some embodiments, the short-sgRNA comprises a 3′ terminus comprising a 3′ tail, which follows and is 3′ of the conserved portion of a short-sgRNA. In some embodiments, the 3′ tail comprises between 1 and about 20 nucleotides, between 1 and about 15 nucleotides, between 1 and about 10 nucleotides, between 1 and about 5 nucleotides, between 1 and about 4 nucleotides, between 1 and about 3 nucleotides, and between 1 and about 2 nucleotides. In some embodiments, the 3′ tail comprises about 1, 2, 3, 4, 5, 6, 7, 8, 9, or 10 nucleotides. In some embodiments, the 3′ tail comprises 1, 2, 3, 4, 5, 6, 7, 8, 9, or 10 nucleotides. In some embodiments, the 3′ tail comprises 1 nucleotide. In some embodiments, the 3′ tail comprises 2 nucleotides. In some embodiments, the 3′ tail comprises 3 nucleotides. In some embodiments, the 3′ tail comprises 4 nucleotides. In some embodiments, the 3′ tail comprises about 1-2, 1-3, 1-4, 1-5, 1-7, 1-10, at least 1-5, at least 1-3, at least 1-4, at least 1-5, at least 1-5, at least 1-7, or at least 1-10 nucleotides.

In some embodiments, the 3′ tail comprising between 1 and 20 nucleotides and follows the 3′ end of the conserved portion of a short-sgRNA.

In some embodiments, the 3′ tail comprises or further comprises one or more of a protective end modification, a phosphorothioate (PS) linkage between nucleotides, a 2′-OMe modified nucleotide, a 2′-O-moe modified nucleotide, a 2′-F modified nucleotide, an inverted abasic modified nucleotide, and a combination thereof.

In some embodiments, the 3′ tail comprises or further comprises one or more phosphorothioate (PS) linkages between nucleotides. In some embodiments, the 3′ tail comprises or further comprises one or more 2′-OMe modified nucleotides. In some embodiments, the 3′ tail comprises or further comprises one or more 2′-O-moe modified nucleotides. In some embodiments, the 3′ tail comprises or further comprises one or more 2′-F modified nucleotide. In some embodiments, the 3′ tail comprises or further comprises one or more an inverted abasic modified nucleotides. In some embodiments, the 3′ tail comprises or further comprises one or more protective end modifications. In some embodiments, the 3′ tail comprises or further comprises a combination of one or more of a phosphorothioate (PS) linkage between nucleotides, a 2′-OMe modified nucleotide, a 2′-O-moe modified nucleotide, a 2′-F modified nucleotide, and an inverted abasic modified nucleotide.

In some embodiments, the short-sgRNA does not comprise a 3′ tail.

5′ Terminus Region Modifications

In some embodiments, the 5′ terminus region is modified, for example, the first 1, 2, 3, 4, 5, 6, or 7 nucleotides of the short-sgRNA are modified. Throughout, this modification may be referred to as a “5′ end modification”. In some embodiments, the first 1, 2, 3, 4, 5, 6, or 7 nucleotides of the 5′ terminus region comprise more than one modification. In some embodiments, at least one of the terminal (i.e., first) 1, 2, 3, 4, 5, 6, or 7 nucleotides at the 5′ end are modified. In some embodiments, at least two of the terminal 1, 2, 3, 4, 5, 6, or 7 nucleotides at the 5′ terminus region are modified. In some embodiments, at least three of the terminal 1, 2, 3, 4, 5, 6, or 7 nucleotides at the 5′ terminus region are modified. In some embodiments, the 5′ end modification is a 5′ protective end modification.

In some embodiments, both the 5′ and 3′ terminus regions (e.g., ends) of the short-sgRNA are modified. In some embodiments, only the 5′ terminus region of the short-sgRNA is modified. In some embodiments, only the 3′ terminus region (plus or minus a 3′ tail) of the conserved portion of a short sgRNA is modified.

In some embodiments, the short-sgRNA comprises modifications at 1, 2, 3, 4, 5, 6, or 7 of the first 7 nucleotides at a 5′ terminus region of the short-sgRNA. In some embodiments, the short-sgRNA comprises modifications at 1, 2, 3, 4, 5, 6, or 7 of the 7 terminal nucleotides at a 3′ terminus region. In some embodiments, 2, 3, or 4 of the first 4 nucleotides at the 5′ terminus region, and/or 2, 3, or 4 of the terminal 4 nucleotides at the 3′ terminus region are modified. In some embodiments, 2, 3, or 4 of the first 4 nucleotides at the 5′ terminus region are linked with phosphorothioate (PS) bonds.

In some embodiments, the modification to the 5′ terminus and/or 3′ terminus comprises a 2′-O-methyl (2′-O-Me) or 2′-O-(2-methoxyethyl) (2′-O-moe) modification. In some embodiments, the modification comprises a 2′-fluoro (2′-F) modification to a nucleotide. In some embodiments, the modification comprises a phosphorothioate (PS) linkage between nucleotides. In some embodiments, the modification comprises an inverted abasic nucleotide. In some embodiments, the modification comprises a protective end modification. In some embodiments, the modification comprises a more than one modification selected from protective end modification, 2′-O-Me, 2′-O-moe, 2′-fluoro (2′-F), a phosphorothioate (PS) linkage between nucleotides, and an inverted abasic nucleotide. In some embodiments, an equivalent modification is encompassed.

In some embodiments, the short-sgRNA comprises one or more phosphorothioate (PS) linkages between the first one, two, three, four, five, six, or seven nucleotides at the 5′ terminus. In some embodiments, the short-sgRNA comprises one or more PS linkages between the last one, two, three, four, five, six, or seven nucleotides at the 3′ terminus. In some embodiments, the short-sgRNA comprises one or more PS linkages between both the last one, two, three, four, five, six, or seven nucleotides at the 3′ terminus and the first one, two, three, four, five, six, or seven nucleotides from the 5′ end of the 5′ terminus. In some embodiments, in addition to PS linkages, the 5′ and 3′ terminal nucleotides may comprise 2′-O-Me, 2′-O-moe, or 2′-F modified nucleotides.

In some embodiments, the short-sgRNA comprises a 5′ end modification, e.g., wherein the first nucleotide of the guide region is modified. In some embodiments, the short-sgRNA comprises a 5′ end modification, wherein the first nucleotide of the guide region comprises a 5′ protective end modification.

In some embodiments, the 5′ end modification comprises a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.

In some embodiments, the 5′ end modification comprises or further comprises a 2′-O-methyl (2′-O-Me) modified nucleotide.

In some embodiments, the 5′ end modification comprises or further comprises a 2′-fluoro (2′-F) modified nucleotide.

In some embodiments, the 5′ end modification comprises or further comprises a phosphorothioate (PS) linkage between nucleotides.

In some embodiments, the 5′ end modification comprises or further comprises an inverted abasic modified nucleotide.

In some embodiments, the 5′ end modification comprises or further comprises a modification of any one or more of nucleotides 1-7 of the guide region of a short-sgRNA. In some embodiments, the 5′ end modification comprises or further comprises one modified nucleotide. In some embodiments, the 5′ end modification comprises or further comprises two modified nucleotides. In some embodiments, the 5′ end modification comprises or further comprises three modified nucleotides. In some embodiments, the 5′ end modification comprises or further comprises four modified nucleotides. In some embodiments, the 5′ end modification comprises or further comprises five modified nucleotides. In some embodiments, the 5′ end modification comprises or further comprises six modified nucleotides. In some embodiments, the 5′ end modification comprises or further comprises seven modified nucleotides.

In some embodiments, the 5′ end modification comprises or further comprises a modification of between 1 and 7, between 1 and 5, between 1 and 4, between 1 and 3, or between 1 and 2 nucleotides.

In some embodiments, the 5′ end modification comprises or further comprises modifications of 1, 2, 3, 4, 5, 6, or 7 nucleotides from the 5′ end. In some embodiments, the 5′ end modification comprises or further comprises modifications of about 1-3, 1-4, 1-5, 1-6, or 1-7 nucleotides from the 5′ end.

In some embodiments, the 5′ end modification comprises or further comprises modifications at the first nucleotide at the 5′ end of the short-sgRNA. In some embodiments, the 5′ end modification comprises or further comprises modifications at the first and second nucleotide from the 5′ end of the short-sgRNA. In some embodiments, the 5′ end modification comprises or further comprises modifications at the first, second, and third nucleotide from the 5′ end of the short-sgRNA. In some embodiments, the 5′ end modification comprises or further comprises modifications at the first, second, third, and fourth nucleotide from the 5′ end of the short-sgRNA. In some embodiments, the 5′ end modification comprises or further comprises modifications at the first, second, third, fourth, and fifth nucleotide from the 5′ end of the short-sgRNA. In some embodiments, the 5′ end modification comprises or further comprises modifications at the first, second, third, fourth, fifth, and sixth nucleotide from the 5′ end of the short-sgRNA. In some embodiments, the 5′ end modification comprises or further comprises modifications at the first, second, third, fourth, fifth, sixth, and seventh nucleotide from the 5′ end of the short-sgRNA.

In some embodiments, the 5′ end modification comprises or further comprises a phosphorothioate (PS) linkage between nucleotides, and/or a 2′-O-Me modified nucleotide, and/or a 2′-O-moe modified nucleotide, and/or a 2′-F modified nucleotide, and/or an inverted abasic modified nucleotide, and/or combinations thereof.

In some embodiments, the 5′ end modification comprises or further comprises 1, 2, 3, 4, 5, 6, and/or 7 PS linkages between nucleotides. In some embodiments, the 5′ end modification comprises or further comprises about 1-2, 1-3, 1-4, 1-5, 1-6, or 1-7 PS linkages between nucleotides.

In some embodiments, the 5′ end modification comprises or further comprises at least one PS linkage, wherein if there is one PS linkage, the linkage is between nucleotides 1 and 2 of the guide region.

In some embodiments, the 5′ end modification comprises or further comprises at least two PS linkages, and the linkages are between nucleotides 1 and 2, and 2 and 3 of the guide region.

In some embodiments, the 5′ end modification comprises or further comprises PS linkages between any one or more of nucleotides 1 and 2, 2 and 3, and 3 and 4 of the guide region.

In some embodiments, the 5′ end modification comprises or further comprises PS linkages between any one or more of nucleotides 1 and 2, 2 and 3, 3 and 4, and 4 and 5 of the guide region.

In some embodiments, the 5′ end modification comprises or further comprises PS linkages between any one or more of nucleotides 1 and 2, 2 and 3, 3 and 4, 4 and 5, and 5 and 6 of the guide region.

In some embodiments, the 5′ end modification comprises or further comprises PS linkages between any one or more of nucleotides 1 and 2, 2 and 3, 3 and 4, 4 and 5, 5 and 6, and 7 and 8 of the guide region.

In some embodiments, the 5′ end modification comprises or further comprises a modification of one or more of nucleotides 1-7 of the guide region, wherein the modification is a PS linkage, inverted abasic nucleotide, 2′-O-Me, 2′-O-moe, 2′-F, and/or combinations thereof.

In some embodiments, the 5′ end modification comprises or further comprises a modification to the first nucleotide of the guide region with 2′-O-Me, 2′-O-moe, 2′-F, or combinations thereof, and an optional PS linkage to the next nucleotide;

In some embodiments, the 5′ end modification comprises or further comprises a modification to the first and/or second nucleotide of the guide region with 2′-O-Me, 2′-O-moe, 2′-F, or combinations thereof, and optionally one or more PS linkages between the first and second nucleotide and/or between the second and third nucleotide.

In some embodiments, the 5′ end modification comprises or further comprises a modification to the first, second, and/or third nucleotides of the variable region with 2′-O-Me, 2′-O-moe, 2′-F, or combinations thereof, and optionally one or more PS linkages between the first and second nucleotide, between the second and third nucleotide, and/or between the third and the fourth nucleotide.

In some embodiments, the 5′ end modification comprises or further comprises a modification to the first, second, third, and/or fourth nucleotides of the variable region with 2′-O-Me, 2′-O-moe, 2′-F, or combinations thereof, and optionally one or more PS linkages between the first and second nucleotide, between the second and third nucleotide, between the third and the fourth nucleotide, and/or between the fourth and the fifth nucleotide.

In some embodiments, the 5′ end modification comprises or further comprises a modification to the first, second, third, fourth, and/or fifth nucleotides of the variable region with 2′-O-Me, 2′-O-moe, 2′-F, or combinations thereof, and optionally one or more PS linkages between the first and second nucleotide, between the second and third nucleotide, between the third and the fourth nucleotide, between the fourth and the fifth nucleotide, and/or between the fifth and the sixth nucleotide.

In some embodiments, a short-sgRNA is provided comprising a 5′ end modification, wherein the 5′ end modification comprises a 5′ end modification as shown in any one of SEQ ID Nos: 1-54.

In some embodiments, the sgRNA comprises a 5′ end modification comprising a 5′ protective end modification. In some embodiments, a short-sgRNA is provided comprising a 5′ end modification, wherein the 5′ end modification comprises 2′-OMe modified nucleotides at nucleotides 1, 2, and 3 of the guide region.

In some embodiments, a short-sgRNA is provided comprising a 5′ end modification, wherein the 5′ end modification comprises 2′-OMe modified nucleotides at nucleotides 1, 2, and 3 of the guide region and PS linkages between nucleotides 1 and 2, 2 and 3, and 3 and 4 of the guide region.

In some embodiments, a short-sgRNA is provided comprising a 5′ end modification, wherein the 5′ end modification comprises 2′-OMe modified nucleotides at nucleotides 1, 2, 3, 4, and 5 of the guide region.

In some embodiments, a short-sgRNA is provided comprising a 5′ end modification, wherein the 5′ end modification comprises 2′-OMe modified nucleotides at nucleotides 1, 2, 3, 4, and 5 of the guide region and PS linkages between nucleotides 1 and 2, 2 and 3, 3 and 4, 4 and 5, and 5 and 6 of the guide region.

In some embodiments, a short-sgRNA is provided comprising a 5′ end modification, wherein the 5′ end modification comprises 2′O-moe modified nucleotides at nucleotides 1, 2, and 3 of the guide region.

In some embodiments, a short-sgRNA is provided comprising a 5′ end modification, wherein the 5′ end modification comprises 2′O-moe modified nucleotides at nucleotides 1, 2, and 3 of the guide region and PS linkages between nucleotides 1 and 2, 2 and 3, and 3 and 4 of the guide region.

In some embodiments, a short-sgRNA is provided comprising a 5′ end modification, wherein the 5′ end modification comprises an inverted abasic modified nucleotide at nucleotide 1 of the guide region.

In some embodiments, a short-sgRNA is provided comprising a 5′ end modification, wherein the 5′ end modification comprises an inverted abasic modified nucleotide at nucleotide 1 of the guide region and 2′-OMe modified nucleotides at nucleotides 1, 2, and 3 of the guide region.

In some embodiments, a short-sgRNA is provided comprising a 5′ end modification, wherein the 5′ end modification comprises an inverted abasic modified nucleotide at nucleotide 1 of the guide region, 2′-OMe modified nucleotides at nucleotides 1, 2, and 3 of the guide region, and PS linkages between nucleotides 1 and 2, 2 and 3, 3 and 4, 4 and 5, and 5 and 6 of the guide region.

In some embodiments, a short-sgRNA is provided comprising a 5′ end modification and a 3′ end modification. In some embodiments, the sgRNA comprises modified nucleotides at the 5′ and 3′ terminus, and modified nucleotides in one or more other regions described in Table 3.

In some embodiments, the sgRNA comprises modified nucleotides that are not at the 5′ or 3′ ends. Exemplary patterns of modifications are described below and in Table 1.

Upper Stem Modifications

In some embodiments, a short-sgRNA is provided comprising an upper stem modification, wherein the upper stem modification comprises a modification to any one or more of US1-US12 in the upper stem region.

In some embodiments, a short-sgRNA is provided comprising an upper stem modification, wherein the upper stem modification comprises a modification of at least 1, 2, 3, 4, 5, 6, 7, 8, 9, 10, 11, or all 12 nucleotides in the upper stem region.

In some embodiments, a short-sgRNA is provided comprising an upper stem modification, wherein the upper stem modification comprises a modification of about 1-2, 1-3, 1-4, 1-5, 1-6, 1-7, 1-8, 1-9, 1-10, or 1-12 nucleotides in the upper stem region.

In some embodiments, an sgRNA is provided comprising an upper stem modification, wherein the upper stem modification comprises 1, 2, 3, 4, or 5 YA modifications in a YA site. In some embodiments, an sgRNA is provided comprising an upper stem modification, wherein the upper stem modification comprises at least 1, 2, 3, 4, or 5 YA modifications. In some embodiments, an sgRNA is provided comprising an upper stem modification, wherein the upper stem modification comprises one YA modification. In some embodiments, an sgRNA is provided comprising an upper stem modification, wherein the upper stem modification comprises 2 YA modifications. In some embodiments, the upper stem modification comprises 3 YA modifications. In some embodiments, one or more YA modifications are in a YA site. In some embodiments, one or more YA modifications are distal to a YA site.

In some embodiments, a short-sgRNA is provided comprising an upper stem modification, wherein the upper stem modification comprises a 2′-OMe modified nucleotide. In some embodiments, a short-sgRNA is provided comprising an upper stem modification, wherein the upper stem modification comprises a 2′-O-moe modified nucleotide. In some embodiments, a short-sgRNA is provided comprising an upper stem modification, wherein the upper stem modification comprises a 2′-F modified nucleotide.

In some embodiments, a short-sgRNA is provided comprising an upper stem modification, wherein the upper stem modification comprises a 2′-OMe modified nucleotide, a 2′-O-moe modified nucleotide, a 2′-F modified nucleotide, and/or combinations thereof.

In some embodiments, the sgRNA comprises an upper stem modification as shown in any one of the sequences in Table 1. In some embodiments, such an upper stem modification is combined with a 5′ protective end modification, e.g. as shown for the corresponding sequence in Table 1. In some embodiments, such an upper stem modification is combined with a 3′ protective end modification, e.g. as shown for the corresponding sequence in Table 1. In some embodiments, such an upper stem modification is combined with 5′ and 3′ end modifications as shown for the corresponding sequence in Table 1.

In some embodiments, the short-sgRNA comprises a 5′ end modification and an upper stem modification. In some embodiments, the short-sgRNA comprises a 3′ end modification and an upper stem modification. In some embodiments, the short-sgRNA comprises a 5′ end modification, a 3′ end modification and an upper stem modification.

Hairpin Modifications

In some embodiments, the short-sgRNA comprises a modification in the hairpin region. In some embodiments, the hairpin region modification comprises at least one modified nucleotide selected from a 2′-O-methyl (2′-OMe) modified nucleotide, a 2′-fluoro (2′-F) modified nucleotide, and/or combinations thereof.

In some embodiments, the hairpin region modification is in the hairpin 1 region. In some embodiments, the hairpin region modification is in the hairpin 2 region. In some embodiments, modifications are within the hairpin 1 and hairpin 2 regions, optionally wherein the “n” between hairpin 1 and 2 is also modified.

In some embodiments, a short-sgRNA is provided comprising a hairpin modification, wherein the hairpin modification comprises 1, 2, or 3 YA modifications in a YA site. In some embodiments, a short-sgRNA is provided comprising a hairpin modification, wherein the hairpin modification comprises at least 1, 2, 3, 4, 5, or 6 YA modifications. In some embodiments, a short-sgRNA is provided comprising a hairpin modification, wherein the hairpin modification comprises one YA modification. In some embodiments, a short-sgRNA is provided comprising a hairpin modification, wherein hairpin modification comprises 2 YA modifications. In some embodiments, the hairpin modification comprises 3 YA modifications. In some embodiments, one or more YA modifications are in a YA site. In some embodiments, one or more YA modifications are distal to a YA site.

In some embodiments, the hairpin modification comprises or further comprises a 2′-O-methyl (2′-OMe) modified nucleotide.

In some embodiments, the hairpin modification comprises or further comprises a 2′-fluoro (2′-F) modified nucleotide.

In some embodiments, the hairpin region modification comprises at least one modified nucleotide selected from a 2′H modified nucleotide (DNA), PS modified nucleotide, a YA modification, a 2′-O-methyl (2′-O-Me) modified nucleotide, a 2′-fluoro (2′-F) modified nucleotide, and/or combinations thereof.

In some embodiments, the short-sgRNA comprises a 3′ end modification, and a modification in the hairpin region.

In some embodiments, the short-sgRNA comprises a 5′ end modification, and a modification in the hairpin region.

In some embodiments, the short-sgRNA comprises an upper stem modification, and a modification in the hairpin region.

In some embodiments, the short-sgRNA comprises a hairpin modification as shown in any one of the sequences in Table 1. In some embodiments, such a hairpin modification is combined with a 5′ end modification as shown for the corresponding sequence in Table 1. In some embodiments, such a hairpin modification is combined with a 3′ end modification as shown for the corresponding sequence in Table 1. In some embodiments, such a hairpin modification is combined with 5′ and 3′ end modifications as shown for the corresponding sequence in Table 1.

In some embodiments, the short-sgRNA comprises a 3′ end modification, a modification in the hairpin region, an upper stem modification, and a 5′ end modification.

Exemplary Modified Short-sgRNAs

In some embodiments, the short-sgRNAs described herein comprise or consist of any of the sequences shown in Table 1. Further, short-sgRNAs are encompassed that comprise the modifications of any of the sequences shown in Table 1, and identified therein by SEQ ID No. That is, the nucleotides may be the same or different, but the modification pattern shown may be the same or similar to a modification pattern of a guide sequence of Table 1. A modification pattern includes the relative position and identity of modifications of the short-sgRNA (e.g. 5′ terminus region, lower stem region, bulge region, upper stem region, nexus region, hairpin 1 region, hairpin 2 region, 3′ tail region).

In some embodiments, the modification pattern contains at least 50%, 55%, 60%, 70%, 75%, 80%, 85%, 90%, 95%, 96%, 97%, 98%, and 99% of the modifications of any one of the sequences shown in the sequence column of Table 1, or over one or more regions of the sequence. In some embodiments, the modification pattern is at least 50%, 55%, 60%, 70%, 75%, 80%, 85%, 90%, 95%, 96%, 97%, 98%, and 99% identical to the modification pattern of any one of the sequences shown in the sequence column of Table 1. In some embodiments, the modification pattern is at least 50%, 55%, 60%, 70%, 75%, 80%, 85%, 90%, 95%, 96%, 97%, 98%, and 99% identical over one or more (e.g., 1, 2, 3, 4, 5, 6, 7, or 8) regions of the sequence shown in Table 1, e.g., a 5′ terminus region, lower stem region, bulge region, upper stem region, nexus region, hairpin 1 region, hairpin 2 region, and/or 3′ terminus region.

For example, in some embodiments, a short-sgRNA is encompassed wherein the modification pattern is least 50%, 55%, 60%, 70%, 75%, 80%, 85%, 90%, 95%, 96%, 97%, 98%, and 99% identical to the modification pattern of a sequence over the 5′ terminus region. In some embodiments, a short-sgRNA is encompassed wherein the modification pattern is least 50%, 55%, 60%, 70%, 75%, 80%, 85%, 90%, 95%, 96%, 97%, 98%, and 99% identical over the lower stem. In some embodiments, a short-sgRNA is encompassed wherein the modification pattern is least 50%, 55%, 60%, 70%, 75%, 80%, 85%, 90%, 95%, 96%, 97%, 98%, and 99% identical over the bulge. In some embodiments, a short-sgRNA is encompassed wherein the modification pattern is least 50%, 55%, 60%, 70%, 75%, 80%, 85%, 90%, 95%, 96%, 97%, 98%, and 99% identical over the upper stem. In some embodiments, a short-sgRNA is encompassed wherein the modification pattern is least 50%, 55%, 60%, 70%, 75%, 80%, 85%, 90%, 95%, 96%, 97%, 98%, and 99% identical over the nexus. In some embodiments, a short-sgRNA is encompassed wherein the modification pattern is least 50%, 55%, 60%, 70%, 75%, 80%, 85%, 90%, 95%, 96%, 97%, 98%, and 99% identical over the hairpin 1. In some embodiments, a short-sgRNA is encompassed wherein the modification pattern is least 50%, 55%, 60%, 70%, 75%, 80%, 85%, 90%, 95%, 96%, 97%, 98%, and 99% identical over the hairpin 2. In some embodiments, a short-sgRNA is encompassed wherein the modification pattern is least 50%, 55%, 60%, 70%, 75%, 80%, 85%, 90%, 95%, 96%, 97%, 98%, and 99% identical over the 3′ terminus. In some embodiments, the modification pattern differs from the modification pattern of a sequence of Table 1, or a region (e.g. 5′ terminus, lower stem, bulge, upper stem, nexus, hairpin 1, hairpin 2, 3′ terminus) of such a sequence, at 0, 1, 2, 3, 4, 5, or 6 nucleotides. In some embodiments, the short-sgRNA comprises modifications that differ from the modifications of a sequence of Table 1, at 0, 1, 2, 3, 4, 5, or 6 nucleotides. In some embodiments, the short-sgRNA comprises modifications that differ from modifications of a region (e.g. 5′ terminus, lower stem, bulge, upper stem, nexus, hairpin 1, hairpin 2, 3′ terminus) of a sequence of Table 1, at 0, 1, 2, 3, 4, 5, or 6 nucleotides.

In some embodiments, the short-sgRNA comprises a 2′-O-methyl (2′-O-Me) modified nucleotide. In some embodiments, the short-sgRNA comprises a 2′-O-(2-methoxyethyl) (2′-O-moe) modified nucleotide. In some embodiments, the short-sgRNA comprises a 2′-fluoro (2′-F) modified nucleotide. In some embodiments, the short-sgRNA comprises a phosphorothioate (PS) bond between nucleotides. In some embodiments, the sgRNA comprises a YA modification.

In some embodiments, the short-sgRNA comprises a 5′ end modification, a 3′ end modification, or 5′ and 3′ end modification, such as a protective end modification. In some embodiments, the 5′ end modification comprises a phosphorothioate (PS) bond between nucleotides. In some embodiments, the 5′ end modification comprises a 2′-O-methyl (2′-O-Me), 2′-O-(2-methoxyethyl) (2′-O-moe), and/or 2′-fluoro (2′-F) modified nucleotide. In some embodiments, the 5′ end modification comprises at least one phosphorothioate (PS) bond and one or more of a 2′-O-methyl (2′-O-Me), 2′-O-(2-methoxyethyl) (2′-O-moe), and/or 2′-fluoro (2′-F) modified nucleotide. The end modification may comprise a phosphorothioate (PS), 2′-O-methyl (2′-O-Me), 2′-O-(2-methoxyethyl) (2′-O-moe), and/or 2′-fluoro (2′-F) modification. Equivalent end modifications are also encompassed by embodiments described herein. In some embodiments, the short-sgRNA comprises an end modification in combination with a modification of one or more regions of the short-sgRNA.

Modified short-sgRNAs comprising combinations of 5′ end modifications, 3′ end modifications, upper stem modifications, hairpin modifications, and 3′ terminus modifications, as described above, are encompassed. Exemplary modified short-sgRNAs are described below.

In some embodiments, the invention comprises a short-sgRNA comprising or consisting of any one of the sequences described in SEQ ID Nos: 1-54, 201-254, and 301-354.

In some embodiments, a short-sgRNA is provided comprising any one of the modified sequences of SEQ ID Nos: 201-254, and 301-354, wherein the short-sgRNA further comprises a guide region that is complementary to a target sequence, and directs a Cas9 to its target for cleavage. In some instances, the invention comprises short-sgRNA comprising nucleic acids having at least 99, 98, 97, 96, 95, 94, 93, 92, 91, 90, 85, 80, 75, or 70% identity to the nucleic acids of any one of SEQ ID Nos: 1-54, 201-254, and 301-354, wherein the modification pattern is identical to the modification pattern shown in the reference sequence identifier in Table 1. In some embodiments, the short-sgRNA further comprises three phosphorothioate (PS) bonds linking the first four nucleotides at the 5′ terminus and three PS bonds linking the last four nucleotides at the 3′ terminus.

In some embodiments, the short-sgRNA comprises modifications at 1, 2, 3, or 4 of the first 4 nucleotides at its 5′ end. In some embodiments, the first three or four nucleotides at the 5′ terminus, and the last three or four nucleotides at the 3′ terminus are modified. In some embodiments, the first four nucleotides at the 5′ end, and the last four nucleotides at the 3′ terminus are linked with phosphorothioate (PS) bonds. In some embodiments, the modification comprises 2′-O-Me. In some embodiments, the modification comprises 2′-F. In some embodiments, the modification comprises 2′-O-moe.

In some embodiments, the short-sgRNA comprises, if the nucleotide mentioned is present in the short-sgRNA, modifications at 1, 2, 3, or 4 of the first 4 nucleotides at the 5′ end. In some embodiments, the short-sgRNA comprises modifications at 1, 2, 3, or 4 of the last 4 nucleotides at the 3′ end (3′ tail or conserved portion of an sgRNA). In some embodiments, the first four nucleotides at the 5′ terminus and the last four nucleotides at the 3′ terminus are linked with a PS bond, and the first three nucleotides at the 5′ terminus and the last three nucleotides at the 3′ terminus comprise 2′-O-Me or 2′-O-moe modifications.

In some embodiments, the first four nucleotides at the 5′ terminus and the last four nucleotides at the 3′ terminus are linked with a PS bond, and the first three nucleotides at the 5′ terminus and the last three nucleotides at the 3′ terminus comprise 2′-F modifications.

In some embodiments, a short-sgRNA is provided, if the nucleotide mentioned is present in the short-sgRNA, wherein LS1, LS6, LS7, LS8, LS11, and LS12 are modified with 2′-O-Me. In some embodiments, each of the nucleotides in the bulge region of the short-sgRNA are modified with 2′-O-Me. In some embodiments, each of the nucleotides in the upper stem region of the short-sgRNA are modified with 2′-O-Me. In some embodiments, N16, N17, and N18 in the nexus region of the short-sgRNA are modified with 2′-O-Me. In some embodiments, each of the nucleotides remaining in the hairpin 1 region of the short-sgRNA are modified with 2′-O-Me. In some embodiments, each of the nucleotides remaining in the hairpin 2 region of the short-sgRNA are modified with 2′-O-Me.

In some embodiments, a short-sgRNA comprising a 5′ end modification and one or more modifications in one or more of: the upper stem region; the hairpin 1 region; and the hairpin 2 region is provided, wherein the 5′ end modification comprises at least two phosphorothioate linkages within the first seven nucleotides of the 5′ terminus.

In some embodiments, a short-sgRNA comprising a 5′ end modification and one or more modifications in one or more of: the upper stem region; the hairpin 1 region; and the hairpin 2 region is provided, wherein the 5′ end modification comprises one or more phosphorothioate linkages at the 5′ end. In some embodiments, one or more phorphorothioate bonds link the 5′ terminal nucleotides.

In some embodiments, a short-sgRNA comprising a 5′ end modification and one or more modifications in one or more of: the upper stem region; the hairpin 1 region; and the hairpin 2 region is provided, wherein the 5′ end modification comprises one or more phosphorothioate linkages within the first seven nucleotides of the 5′ terminus.

In some embodiments, the invention comprises a short-sgRNA comprising any one of the modified sequences of SEQ ID Nos: 201-254, and 301-354, wherein the short-sgRNA further comprises a 5′ guide region that is at least partially complementary to a target sequence, and optionally directs a Cas9 to its target for cleavage.

In some embodiments, the invention comprises a short-sgRNA comprising nucleotides having at least 99, 98, 97, 96, 95, 94, 93, 92, 91, 90, 85, 80, 75, or 70% identity to the nucleotides of any one of SEQ ID Nos: 1-54, 201-254, and 301-354, wherein the modification pattern is identical to the modification pattern shown in the reference sequence identifier. That is, the nucleotides A, U, C, and G may differ by 99, 98, 97, 96, 95, 94, 93, 92, 91, 90, 85, 80, 75, or 70% compared to what is shown in in the sequences, but the modification remains unchanged.

In some embodiments, a short-sgRNA is provided comprising, if the nucleotide mentioned is present in the short guide, 2′-O-Me modified nucleotides at: the first three nucleotides in the 5′ terminus; LS1, LS6, LS7, LS8, LS11, and LS12 in the lower stem; B1 and B2 in the bulge region; each of the nucleotides in the upper stem region; N16, N17, and N18 in the nexus region; each of the nucleotides in the hairpin 1 region; one nucleotide between hairpin 1 and hairpin 2; each of the nucleotides in the hairpin 2 region; and the last four nucleotides at the 3′ terminus. In some embodiments, the sgRNA further comprises three PS bonds between the first four nucleotides at the 5′ terminus and three PS bonds between the last four nucleotides at the 3′ terminus.

In some embodiments, a short-sgRNA is provided comprising, if the nucleotide mentioned is present in the short guide, 2′-O-Me modified nucleotides at: the first three nucleotides in the 5′ terminus; LS1, LS6, LS7, LS8, LS11, and LS12 in the lower stem; B1-B6 in the bulge region; each of the nucleotides in the upper stem region; N16, N17, and N18 in the nexus region; each of the nucleotides in the hairpin 1 region; one nucleotide between hairpin 1 and hairpin 2; each of the nucleotides in the hairpin 2 region; and the last four nucleotides at the 3′ terminus. In some embodiments, the sgRNA further comprises three PS bonds between the first four nucleotides at the 5′ terminus and three PS bonds between the last four nucleotides at the 3′ terminus.

In some embodiments, a short-sgRNA is provided comprising 2′-F modified nucleotides at: LS9 and LS10 in the lower stem; 15-N18 in the nexus region; H2-9-HS-15 in the hairpin 2 region; and the second to last, third to last, and fourth to last nucleotide in the 3′ terminus region.

In some embodiments, a short-sgRNA is provided comprising 2′-F modified nucleotides at: each nucleotide in the lower stem; 15-N18 in the nexus region; H2-9-HS-15 in the hairpin 2 region; and the second to last, third to last, and fourth to last nucleotide in the 3′ terminus region.

In some embodiments, a short-sgRNA is provided comprising, if the nucleotide mentioned is present in the short guide, 2′-OMe modified nucleotides at LS8, LS10, LS12, H1-2, H1-4, H1-6, H1-8, H1-10, H1-12, H2-1, H2-3, H2-5, H2-7, H2-9, H2-11, H2-13, H2-15, and the last and third to last nucleotides at the 3′ terminus; and 2′-F modifications at LS7, LS9, LS11; H1-1, H1-3, H1-5, H1-7, H1-9, H1-11, H1-13, H2-2, H2-4, H2-6, H2-8, H2-10, H2-12, H2-14, and the second to last and fourth to last nucleotide at the 3′ terminus.

Any of the foregoing modification patterns can be combined with a modification pattern set forth in the embodiments described above, e.g., in the summary section or Table 1, to the extent that they are non-overlapping. In the event that combining a foregoing modification pattern with a modification pattern set forth in the summary section or Table 1 would result in incompatible modifications (e.g., the same position would be both 2′-OMe and 2′-fluoro), the modification set forth in the summary section or Table 1 controls.

### Compositions and Kits

Compositions comprising any of the gRNAs (e.g., sgRNAs, short-sgRNAs, dgRNAs, or crRNAs) described herein and a carrier, excipient, diluent, or the like are encompassed. In some instances, the excipient or diluent is inert. In some instances, the excipient or diluent is not inert. In some embodiments, a pharmaceutical formulation is provided comprising any of the gRNAs (e.g., sgRNAs, short-sgRNAs, dgRNAs, or crRNAs) described herein and a pharmaceutically acceptable carrier, excipient, diluent, or the like. In some embodiments, the pharmaceutical formulation further comprises an LNP. In some embodiments, the pharmaceutical formulation further comprises a Cas9 protein or an mRNA encoding a Cas9 protein. In some embodiments, the pharmaceutical formulation comprises any one or more of the gRNAs (e.g., sgRNAs, short-sgRNAs, dgRNAs, or crRNAs), an LNP, and a Cas9 protein or mRNA encoding a Cas9 protein.

Also provided are kits comprising one or more gRNAs (e.g., sgRNAs, short-sgRNAs, dgRNAs, or crRNAs), compositions, or pharmaceutical formulations described herein. In some embodiments, a kit further comprises one or more of a solvent, solution, buffer, each separate from the composition or pharmaceutical formulation, instructions, or desiccant.

Compositions comprising an RNA-Guided DNA Binding Agent or mRNA Encoding RNA-Guided DNA Binding Agent

In some embodiments, compositions or pharmaceutical formulations are provided comprising at least one gRNA (e.g., sgRNA, short-sgRNA, dgRNA, or crRNA) described herein and a nuclease or a nucleic acid (e.g., an mRNA) encoding a nuclease. In some embodiments, the nuclease is an RNA-guided DNA binding agent, such as a Cas protein. In some embodiments, the short-sgRNA together with a Cas protein or nucleic acid (e.g., mRNA) encoding Cas protein is called a Cas RNP. In some embodiments, the RNA-guided DNA binding agent is one that functions with the short-sgRNA to direct a RNA-guided DNA binding agent to a target nucleic acid sequence. In some embodiments, the RNA-guided DNA binding agent is a Cas protein from the Type-II CRISPR/Cas system. In some embodiments, the Cas protein is Cas9. In some embodiments, the Cas9 protein is a wild type Cas9. In some embodiments, the Cas9 protein is derived from the Streptococcus pyogenes Cas9 protein, e.g., a S. pyogenes Cas9 (sypCas9). In some embodiments, compositions are provided comprising at least one short-sgRNA and a nuclease or an mRNA encoding a spyCas9. In some embodiments, the Cas9 protein is not derived from S. pyogenes, but functions in the same way as S. pyogenes Cas9 such that short-sgRNA that is specific to S. pyogenes Cas9 will direct the non-S. pyogenes Cas9 to its target site. In some embodiments, the Cas9 protein is derived from the Staphylococcus aureus Cas9 protein, e.g., a SaCas9. In some embodiments, compositions are provided comprising at least one short-sgRNA and a nuclease or an mRNA encoding a saCas9. In some embodiments, the Cas induces a double strand break in target DNA. Equivalents of spyCas9 and saCas9 protein are encompassed by the embodiments described herein.

RNA-guided DNA binding agents, including Cas9, encompass modified and variants thereof. Modified versions having one catalytic domain, either RuvC or HNH, that is inactive are termed “nickases.” Nickases cut only one strand on the target DNA, thus creating a single-strand break. A single-strand break may also be known as a “nick.” In some embodiments, the compositions and methods comprise nickases. In some embodiments, the compositions and methods comprise a nickase RNA-guided DNA binding agent, such as a nickase Cas9, that induces a nick rather than a double strand break in the target DNA.

In some embodiments, the RNA-guided DNA binding agent may be modified to contain only one functional nuclease domain. For example, the RNA-guided DNA binding agent may be modified such that one of the nuclease domains is mutated or fully or partially deleted to reduce its nucleic acid cleavage activity. In some embodiments, a nickase Cas is used having a RuvC domain with reduced activity. In some embodiments, a nickase Cas is used having an inactive RuvC domain. In some embodiments, a nickase Cas is used having an HNH domain with reduced activity. In some embodiments, a nickase Cas is used having an inactive HNH domain.

In some embodiments, a conserved amino acid within an RNA-guided DNA binding agent nuclease domain is substituted to reduce or alter nuclease activity. In some embodiments, a Cas protein may comprise an amino acid substitution in the RuvC or RuvC-like nuclease domain. Exemplary amino acid substitutions in the RuvC or RuvC-like nuclease domain include D10A (based on the S. pyogenes Cas9 protein). In some embodiments, the Cas protein may comprise an amino acid substitution in the HNH or HNH-like nuclease domain. Exemplary amino acid substitutions in the HNH or HNH-like nuclease domain include E762A, H840A, N863A, H983A, and D986A (based on the spyCas9 protein).

In some embodiments, the RNP complex described herein comprises a nickase or an mRNA encoding a nickase and a pair of gRNAs (one or both of which may be sgRNAs and/or short-sgRNAs) that are complementary to the sense and antisense strands of the target sequence, respectively. In this embodiment, the gRNAs (e.g., sgRNAs and/or short-sgRNAs) direct the nickase to a target sequence and introduce a double stranded break (DSB) by generating a nick on opposite strands of the target sequence (i.e., double nicking). In some embodiments, use of double nicking may improve specificity and reduce off-target effects. In some embodiments, a nickase RNA-guided DNA binding agent is used together with two separate short-sgRNAs targeting opposite strands of DNA to produce a double nick in the target DNA. In some embodiments, a nickase RNA-guided DNA binding agent is used together with two separate gRNAs (e.g., sgRNAs or short-sgRNAs) that are selected to be in close proximity to produce a double nick in the target DNA.

In some embodiments, chimeric Cas proteins are used, where one domain or region of the protein is replaced by a portion of a different protein. In some embodiments, a Cas nuclease domain may be replaced with a domain from a different nuclease such as Fok1. In some embodiments, a Cas protein may be a modified nuclease.

In some embodiments, the Cas protein comprises a fusion protein comprising a catalytically inactive Cas (e.g., Cas9) linked to a heterologous functional domain (see, e.g., WO2014152432). In some embodiments, the catalytically inactive Cas9 is from S. pyogenes. In some embodiments, the catalytically inactive Cas comprises mutations that inactivate the Cas. In some embodiments, the heterologous functional domain is a domain that modifies gene expression, histones, or DNA. In some embodiments, the heterologous functional domain is a transcriptional activation domain or a transcriptional repressor domain.

In some embodiments, the target sequence may be adjacent to a PAM. In some embodiments, the PAM may be adjacent to or within 1, 2, 3, or 4, nucleotides of the 3′ end of the target sequence. The length and the sequence of the PAM may depend on the Cas protein used. For example, the PAM may be selected from a consensus or a particular PAM sequence for a specific Cas9 protein or Cas9 ortholog, including those disclosed in FIG. 1 of Ran et al., Nature 520:186-191 (2015). In some embodiments, the PAM may comprise 2, 3, 4, 5, 6, 7, 8, 9, or 10 nucleotides in length. Non-limiting exemplary PAM sequences include NGG, NAG, NGA, NGAG, NGCG, NNGRRT, TTN, NGGNG, NG, NAAAAN, NNAAAAW, NNNNACA, GNNNCNNA, and NNNNGATT (wherein N is defined as any nucleotide, and W is defined as either A or T, and R is defined as either A or G). In some embodiments, the PAM sequence may be NGG. In some embodiments, the PAM sequence may be NGGNG. In some embodiments, the PAM sequence may be NNAAAAW.

In some embodiments, an nucleic acid (e.g., mRNA) comprising an ORF encoding an RNA-guided DNA binding agent is used which has one or more of the following features. In some embodiments, the ORF encoding the RNA-guided DNA-binding agent, e.g. a Cas9 nuclease such as an S. pyogenes Cas9, has an adenine content ranging from its minimum adenine content to about 150% of its minimum adenine content. In some embodiments, the adenine content of the ORF is less than or equal to about 145%, 140%, 135%, 130%, 125%, 120%, 115%, 110%, 105%, 104%, 103%, 102%, or 101% of its minimum adenine content. In some embodiments, the ORF has an adenine content equal to its minimum adenine content. In some embodiments, the ORF has an adenine content less than or equal to about 150% of its minimum adenine content. In some embodiments, the ORF has an adenine content less than or equal to about 145% of its minimum adenine content. In some embodiments, the ORF has an adenine content less than or equal to about 140% of its minimum adenine content. In some embodiments, the ORF has an adenine content less than or equal to about 135% of its minimum adenine content. In some embodiments, the ORF has an adenine content less than or equal to about 130% of its minimum adenine content. In some embodiments, the ORF has an adenine content less than or equal to about 125% of its minimum adenine content. In some embodiments, the ORF has an adenine content less than or equal to about 120% of its minimum adenine content. In some embodiments, the ORF has an adenine content less than or equal to about 115% of its minimum adenine content. In some embodiments, the ORF has an adenine content less than or equal to about 110% of its minimum adenine content. In some embodiments, the ORF has an adenine content less than or equal to about 105% of its minimum adenine content. In some embodiments, the ORF has an adenine content less than or equal to about 104% of its minimum adenine content. In some embodiments, the ORF has an adenine content less than or equal to about 103% of its minimum adenine content. In some embodiments, the ORF has an adenine content less than or equal to about 102% of its minimum adenine content. In some embodiments, the ORF has an adenine content less than or equal to about 101% of its minimum adenine content.

In some embodiments, the ORF has an adenine dinucleotide content ranging from its minimum adenine dinucleotide content to 200% of its minimum adenine dinucleotide content. In some embodiments, the adenine dinucleotide content of the ORF is less than or equal to about 195%, 190%, 185%, 180%, 175%, 170%, 165%, 160%, 155%, 150%, 145%, 140%, 135%, 130%, 125%, 120%, 115%, 110%, 105%, 104%, 103%, 102%, or 101% of its minimum adenine dinucleotide content. In some embodiments, the ORF has an adenine dinucleotide content equal to its minimum adenine dinucleotide content. In some embodiments, the ORF has an adenine dinucleotide content less than or equal to about 200% of its minimum adenine dinucleotide content. In some embodiments, the ORF has an adenine dinucleotide content less than or equal to about 195% of its minimum adenine dinucleotide content. In some embodiments, the ORF has an adenine dinucleotide content less than or equal to about 190% of its minimum adenine dinucleotide content. In some embodiments, the ORF has an adenine dinucleotide content less than or equal to about 185% of its minimum adenine dinucleotide content. In some embodiments, the ORF has an adenine dinucleotide content less than or equal to about 180% of its minimum adenine dinucleotide content. In some embodiments, the ORF has an adenine dinucleotide content less than or equal to about 175% of its minimum adenine dinucleotide content. In some embodiments, the ORF has an adenine dinucleotide content less than or equal to about 170% of its minimum adenine dinucleotide content. In some embodiments, the ORF has an adenine dinucleotide content less than or equal to about 165% of its minimum adenine dinucleotide content. In some embodiments, the ORF has an adenine dinucleotide content less than or equal to about 160% of its minimum adenine dinucleotide content. In some embodiments, the ORF has an adenine dinucleotide content less than or equal to about 155% of its minimum adenine dinucleotide content. In some embodiments, the ORF has an adenine dinucleotide content equal to its minimum adenine dinucleotide content. In some embodiments, the ORF has an adenine dinucleotide content less than or equal to about 150% of its minimum adenine dinucleotide content. In some embodiments, the ORF has an adenine dinucleotide content less than or equal to about 145% of its minimum adenine dinucleotide content. In some embodiments, the ORF has an adenine dinucleotide content less than or equal to about 140% of its minimum adenine dinucleotide content. In some embodiments, the ORF has an adenine dinucleotide content less than or equal to about 135% of its minimum adenine dinucleotide content. In some embodiments, the ORF has an adenine dinucleotide content less than or equal to about 130% of its minimum adenine dinucleotide content. In some embodiments, the ORF has an adenine dinucleotide content less than or equal to about 125% of its minimum adenine dinucleotide content. In some embodiments, the ORF has an adenine dinucleotide content less than or equal to about 120% of its minimum adenine dinucleotide content. In some embodiments, the ORF has an adenine dinucleotide content less than or equal to about 115% of its minimum adenine dinucleotide content. In some embodiments, the ORF has an adenine dinucleotide content less than or equal to about 110% of its minimum adenine dinucleotide content. In some embodiments, the ORF has an adenine dinucleotide content less than or equal to about 105% of its minimum adenine dinucleotide content. In some embodiments, the ORF has an adenine dinucleotide content less than or equal to about 104% of its minimum adenine dinucleotide content. In some embodiments, the ORF has an adenine dinucleotide content less than or equal to about 103% of its minimum adenine dinucleotide content. In some embodiments, the ORF has an adenine dinucleotide content less than or equal to about 102% of its minimum adenine dinucleotide content. In some embodiments, the ORF has an adenine dinucleotide content less than or equal to about 101% of its minimum adenine dinucleotide content.

In some embodiments, the ORF has an adenine dinucleotide content ranging from its minimum adenine dinucleotide content to the adenine dinucleotide content that is 90% or lower of the maximum adenine dinucleotide content of a reference sequence that encodes the same protein as the mRNA in question. In some embodiments, the adenine dinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum adenine dinucleotide content of a reference sequence that encodes the same protein as the mRNA in question.

In some embodiments, the ORF has an adenine trinucleotide content ranging from 0 adenine trinucleotides to 1, 2, 3, 4, 5, 6, 7, 8, 9, 10, 20, 30, 40, or 50 adenine trinucleotides (where a longer run of adenines counts as the number of unique three-adenine segments within it, e.g., an adenine tetranucleotide contains two adenine trinucleotides, an adenine pentanucleotide contains three adenine trinucleotides, etc.). In some embodiments, the ORF has an adenine trinucleotide content ranging from 0% adenine trinucleotides to 0.1%, 0.2%, 0.3%, 0.4%, 0.5%, 0.6%, 0.7%, 0.8%, 0.9%, 1%, 1.5%, or 2% adenine trinucleotides, where the percentage content of adenine trinucleotides is calculated as the percentage of positions in a sequence that are occupied by adenines that form part of an adenine trinucleotide (or longer run of adenines), such that the sequences UUUAAA and UUUUAAAA would each have an adenine trinucleotide content of 50%. For example, in some embodiments, the ORF has an adenine trinucleotide content less than or equal to 2%. For example, in some embodiments, the ORF has an adenine trinucleotide content less than or equal to 1.5%. In some embodiments, the ORF has an adenine trinucleotide content less than or equal to 1%. In some embodiments, the ORF has an adenine trinucleotide content less than or equal to 0.9%. In some embodiments, the ORF has an adenine trinucleotide content less than or equal to 0.8%. In some embodiments, the ORF has an adenine trinucleotide content less than or equal to 0.7%. In some embodiments, the ORF has an adenine trinucleotide content less than or equal to 0.6%. In some embodiments, the ORF has an adenine trinucleotide content less than or equal to 0.5%. In some embodiments, the ORF has an adenine trinucleotide content less than or equal to 0.4%. In some embodiments, the ORF has an adenine trinucleotide content less than or equal to 0.3%. In some embodiments, the ORF has an adenine trinucleotide content less than or equal to 0.2%. In some embodiments, the ORF has an adenine trinucleotide content less than or equal to 0.1%. In some embodiments, a nucleic acid is provided that encodes an RNA-guided DNA-binding agent comprising an ORF containing no adenine trinucleotides.

In some embodiments, the ORF has an adenine trinucleotide content ranging from its minimum adenine trinucleotide content to the adenine trinucleotide content that is 90% or lower of the maximum adenine trinucleotide content of a reference sequence that encodes the same protein as the mRNA in question. In some embodiments, the adenine trinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum adenine trinucleotide content of a reference sequence that encodes the same protein as the mRNA in question.

A given ORF can be reduced in adenine content or adenine dinucleotide content or adenine trinucleotide content, for example, by using minimal adenine codons in a sufficient fraction of the ORF. For example, an amino acid sequence for an RNA-guided DNA-binding agent can be back-translated into an ORF sequence by converting amino acids to codons, wherein some or all of the ORF uses the exemplary minimal adenine codons shown below. In some embodiments, at least about 50%, 55%, 60%, 65%, 70%, 75%, 80%, 85%, 90%, 95%, 98%, 99%, or 100% of the codons in the ORF are codons listed in Table 4.

TABLE 4
Exemplary minimal adenine codons

| | Amino Acid | Minimal adenine codon |
|---|---|---|
| A | Alanine | GCU or GCC or GCG |
| G | Glycine | GGU or GGC or GGG |
| V | Valine | GUC or GUU or GUG |
| D | Aspartic acid | GAC or GAU |
| E | Glutamic acid | GAG |
| I | Isoleucine | AUC or AUU |
| T | Threonine | ACU or ACC or ACG |
| N | Asparagine | AAC or AAU |
| K | Lysine | AAG |
| S | Serine | UCU or UCC or UCG |
| R | Arginine | CGU or CGC or CGG |
| L | Leucine | CUG or CUC or CUU |
| P | Proline | CCG or CCU or CCC |
| H | Histidine | CAC or CAU |
| Q | Glutamine | CAG |
| F | Phenylalanine | UUC or UUU |
| Y | Tyrosine | UAC or UAU |
| C | Cysteine | UGC or UGU |
| W | Tryptophan | UGG |
| M | Methionine | AUG |

In some embodiments, a nucleic acid is provided that encodes an RNA-guided DNA-binding agent, e.g. a Cas9 nuclease such as an S. pyogenes Cas9, comprising an ORF consisting of a set of codons of which at least about 75%, 80%, 85%, 90%, 95%, 98%, 99%, or 100% of the codons are codons listed in Table 4. In some embodiments, the ORF has minimal nucleotide homopolymers, e.g., repetitive strings of the same nucleotides. For example, in some embodiments, when selecting a minimal uridine codon from the codons listed in Table 4, a nucleic acid is constructed by selecting the minimal adenine codons that reduce the number and length of nucleotide homopolymers, e.g., selecting GCG instead of GCC for alanine or selecting GGC instead of GGG for glycine.

In any of the foregoing embodiments, the nucleic acid may be an mRNA.

In some embodiments, at least 75%, 80%, 85%, 90%, 95%, 96%, 97%, 98%, 99%, or 100% of the codons in an ORF are codons from a codon set shown in Table 5 (e.g., the low U, low A, or low A/U codon set). The codons in the low U, low A, and low A/U sets use codons that minimize the indicated nucleotides while also using codons corresponding to highly expressed tRNAs where more than one option is available. In some embodiments, at least 75%, 80%, 85%, 90%, 95%, 96%, 97%, 98%, 99%, or 100% of the codons in an ORF are codons from the low U codon set shown in Table 5. In some embodiments, at least 75%, 80%, 85%, 90%, 95%, 96%, 97%, 98%, 99%, or 100% of the codons in an ORF are codons from the low A codon set shown in Table 5. In some embodiments, at least 75%, 80%, 85%, 90%, 95%, 96%, 97%, 98%, 99%, or 100% of the codons in an ORF are codons from the low A/U codon set shown in Table 5.

TABLE 5
Exemplary Codon Sets

| Amino | | | | Long Half |
|---|---|---|---|---|
| Acid | Low U | Low A | Low A/U | Life |
| Gly | GGC | GGC | GGC | GGT |
| Glu | GAG | GAG | GAG | GAA |
| Asp | GAC | GAC | GAC | GAC |
| Val | GTG | GTG | GTG | GTC |
| Ala | GCC | GCC | GCC | GCC |
| Arg | AGA | CGG | CGG | AGA |
| Ser | AGC | TCC | AGC | TCT |
| Lys | AAG | AAG | AAG | AAG |
| Asn | AAC | AAC | AAC | AAC |
| Met | ATG | ATG | ATG | ATG |
| Ile | ATC | ATC | ATC | ATC |
| Thr | ACC | ACC | ACC | ACC |
| Trp | TGG | TGG | TGG | TGG |
| Cys | TGC | TGC | TGC | TGC |
| Tyr | TAC | TAC | TAC | TAC |
| Leu | CTG | CTG | CTG | TTG |
| Phe | TTC | TTC | TTC | TTC |
| Gln | CAG | CAG | CAG | CAA |
| His | CAC | CAC | CAC | CAC |

Exemplary Sequences

In some embodiments, the ORF encoding the RNA-guided DNA binding agent comprises a sequence with at least 90%, 93%, 95%, 96%, 97%, 98%, 99%, 99.5%, or 100% identity to any one of SEQ ID NOs: 3502-3522, 3525, 3526, or 3529-3546; and/or the ORF has at least 90%, 93%, 95%, 96%, 97%, 98%, 99%, 99.5%, or 100% identity to any one of SEQ ID NOs: 3502-3522, 3525, 3526, or 3529-3546 over at least its first 50, 200, 250, or 300 nucleotides, or at least 95% identity to any one of SEQ ID NOs: 3502-3522, 3525, 3526, or 3529-3546 over at least its first 30, 50, 70, 100, 150, 200, 250, or 300 nucleotides; and/or the ORF consists of a set of codons of which at least 95%, 96%, 97%, 98%, 99%, 99.5%, or 100% of the codons are codons listed in Table 4 or 5; and/or the ORF has an adenine content ranging from its minimum adenine content to 123% of the minimum adenine content; and/or the ORF has an adenine dinucleotide content ranging from its minimum adenine dinucleotide content to 150% of the minimum adenine dinucleotide content. In some embodiments, the polynucleotide encoding the RNA-guided DNA binding agent comprises a sequence with at least 95%, 96%, 97%, 98%, 99%, 99.5%, or 100% identity to any one of SEQ ID NOs: 3502-3522, 3525, 3526, or 3529-3546.

In some embodiments, the mRNA comprises a sequence with at least 90% identity to any one of SEQ ID NOs: 3501, 3523, 3524, or 3527, wherein the sequence comprises an ORF encoding an RNA-guided DNA binding agent. In some embodiments, the mRNA comprises a sequence with at least 90% identity to any one of SEQ ID NOs: 3501, 3523, 3524, or 3527, wherein the sequence comprises an ORF encoding an RNA-guided DNA binding agent, wherein the first three nucleotides of SEQ ID NOs: 3501, 3523, 3524, or 3527 are omitted. In some embodiments, the mRNA comprises a sequence with at least 90% identity to any one of SEQ ID NOs: 3501, 3523, 3524, or 3527, wherein the sequence comprises an ORF encoding an RNA-guided DNA binding agent, wherein the first three nucleotides of SEQ ID NOs: 3501, 3523, 3524, or 3527 are omitted and/or the ORF coding sequence contained within SEQ ID NO: 3501, 3523, 3524, or 3527 is substituted with the coding sequence of any one of SEQ ID NOs: 3502-3522, 3525, 3526, or 3529-3546. In some embodiments, any of the foregoing levels of identity is at least 95%, at least 98%, at least 99%, or 100%.

### Methods of Gene Modulation

In some embodiments, any one or more of the gRNAs (e.g., sgRNAs, short-sgRNAs, dgRNAs, or crRNAs), compositions, or pharmaceutical formulations described herein is for use in preparing a medicament for treating or preventing a disease or disorder in a subject.

In some embodiments, the invention comprises a method of treating or preventing a disease or disorder in subject comprising administering any one or more of the gRNAs (e.g., sgRNAs, short-sgRNAs, dgRNAs, or crRNAs), compositions, or pharmaceutical formulations described herein.

In some embodiments, the invention comprises a method or use of modifying a target DNA comprising, administering or delivering any one or more of the gRNAs (e.g., sgRNAs, short-sgRNAs, dgRNAs, or crRNAs), compositions, or pharmaceutical formulations described herein.

In some embodiments, the invention comprises a method or use for modulation of a target gene comprising, administering or delivering any one or more of the gRNAs (e.g., sgRNAs, short-sgRNAs, dgRNAs, or crRNAs), compositions, or pharmaceutical formulations described herein. In some embodiments, the modulation is editing of the target gene. In some embodiments, the modulation is a change in expression of the protein encoded by the target gene.

In some embodiments, the method or use results in gene editing. In some embodiments, the method or use results in a double-stranded break within the target gene. In some embodiments, the method or use results in formation of indel mutations during non-homologous end joining of the DSB. In some embodiments, the method or use results in an insertion or deletion of nucleotides in a target gene. In some embodiments, the insertion or deletion of nucleotides in a target gene leads to a frameshift mutation or premature stop codon that results in a non-functional protein. In some embodiments, the insertion or deletion of nucleotides in a target gene leads to a knockdown or elimination of target gene expression. In some embodiments, the method or use comprises homology directed repair of a DSB. In some embodiments, the method or use further comprises delivering to the cell a template, wherein at least a part of the template incorporates into a target DNA at or near a double strand break site induced by the nuclease.

In some embodiments, the method or use results in gene modulation. In some embodiments, the gene modulation is an increase or decrease in gene expression, a change in methylation state of DNA, or modification of a histone subunit. In some embodiments, the method or use results in increased or decreased expression of the protein encoded by the target gene.

The efficacy of gRNAs (e.g., sgRNAs, short-sgRNAs, dgRNAs, or crRNAs) can be tested in vitro and in vivo. In some embodiments, the invention comprises one or more of the gRNAs (e.g., sgRNAs, short-sgRNAs, dgRNAs, or crRNAs), compositions, or pharmaceutical formulations described herein, wherein the short-sgRNA results in gene modulation when provided to a cell together with Cas9 or mRNA encoding Cas9. In some embodiments, the efficacy of short-sgRNA can be measured in vitro or in vivo.

In some embodiments, the activity of a Cas RNP comprising a short-sgRNA is compared to the activity of a Cas RNP comprising an unmodified sgRNA or a reference sgRNA lacking modifications present in the sgRNA or short-sgRNA, such as YA site modifications.

In some embodiments, the efficiency of a sgRNA or short-sgRNA in increasing or decreasing target protein expression is determined by measuring the amount of target protein.

In some embodiments, the efficiency of editing with specific gRNAs is determined by the editing present at the target location in the genome following delivery of Cas9 and the gRNA. In some embodiments, the efficiency of editing with specific gRNAs is measured by next-generation sequencing. In some embodiments, the editing percentage of the target region of interest is determined. In some embodiments, the total number of sequence reads with insertions or deletions of nucleotides into the target region of interest over the total number of sequence reads is measured following delivery of a gRNA and Cas9.

In some embodiments, the efficiency of editing with specific gRNAs is measured by the presence of insertions or deletions of nucleotides introduced by successful gene editing. In some embodiments, activity of a Cas9 and gRNAs is tested in biochemical assays. In some embodiments, activity of a Cas9 and gRNAs is tested in a cell-free cleavage assay. In some embodiments, activity of a Cas9 and gRNAs is tested in Neuro2A cells.

In some embodiments, the activity of modified gRNAs is measured after in vivo dosing of LNPs comprising modified gRNAs and Cas protein or mRNA encoding Cas protein.

In some embodiments, in vivo efficacy of a gRNA or composition provided herein is determined by editing efficacy measured in DNA extracted from tissue (e.g., liver tissue) after administration of gRNA and Cas9.

In some embodiments, activation of the subject's immune response is measured by serum concentrations of cytokine(s) following in vivo dosing of sgRNA together with Cas9 mRNA or protein (e.g., formulated in a LNP). In some embodiments, the cytokine is interferon-alpha (IFN-alpha), interleukin 6 (IL-6), monocyte chemotactic protein 1 (MCP-1), and/or tumor necrosis factor alpha (TNF-alpha).

In some embodiments, administration of Cas RNP or Cas9 mRNA together with the modified gRNA (e.g., sgRNA, short-sgRNA, or dgRNA) produces lower serum concentration(s) of immune cytokines compared to administration of unmodified sgRNA. In some embodiments, the invention comprises a method of reducing a subject's serum concentration of immune cytokines comprising, administering any one of the gRNAs disclosed herein, wherein the gRNA produces a lower concentration of immune cytokines in a subject's serum as compared to a control gRNA that is not similarly modified.

LNP Delivery of gRNA

Lipid nanoparticles (LNPs) are a well-known means for delivery of nucleotide and protein cargo, and may be used for delivery of the gRNAs (e.g., sgRNAs, short-sgRNAs, dgRNAs, or crRNAs), compositions, or pharmaceutical formulations disclosed herein. In some embodiments, the LNPs deliver nucleic acid, protein, or nucleic acid together with protein.

In some embodiments, the invention comprises a method for delivering any one of the gRNAs (e.g., sgRNAs, short-sgRNAs, dgRNAs, or crRNAs) disclosed herein to a subject, wherein the gRNA is associated with an LNP. In some embodiments, the gRNA/LNP is also associated with a Cas9 or an mRNA encoding Cas9.

In some embodiments, the invention comprises a composition comprising any one of the gRNAs disclosed and an LNP. In some embodiments, the composition further comprises a Cas9 or an mRNA encoding Cas9.

In some embodiments, the LNPs comprise cationic lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). In some embodiments, the LNPs comprise molar ratios of a cationic lipid amine to RNA phosphate (N:P) of about 4.5.

In some embodiments, LNPs associated with the gRNAs disclosed herein are for use in preparing a medicament for treating a disease or disorder.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, the invention comprises a method for delivering any one of the gRNAs disclosed herein to an ex vivo cell, wherein the gRNA is associated with an LNP or not associated with an LNP. In some embodiments, the gRNA/LNP or gRNA is also associated with a Cas9 or an mRNA encoding Cas9.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. “About” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

### EXAMPLES

The following examples are provided to illustrate certain disclosed embodiments and are not to be construed as limiting the scope of this disclosure in any way.

### Example 1—Materials and Methods

Synthetic sgRNA and Short-Single Guide RNA (Short-sgRNA)

Single-guide RNA (sgRNA) and short-single guide (short-sgRNA) was chemically synthesized by commercial vendors or using standard in vitro synthesis techniques with modified nucleotides as provided in Table 1.

In Vitro Transcription (“IVT”) of Cas9 mRNA

Capped and polyadenylated Cas9 mRNA containing N1-methyl pseudouridine was generated by in vitro transcription using a linearized plasmid DNA template and T7 RNA polymerase. Plasmid DNA containing a T7 promoter and a sequence for transcription (for producing mRNA comprising an mRNA described herein (see SEQ ID NOs: 3499, 3500, 3501, 3523, 3524, and 3527 for exemplary transcripts and SEQ ID NOs: 3502-3522, 3525, 3526, and 3529-3546 for exemplary ORFs) was linearized by incubating at 37° C. to complete digestion with XbaI with the following conditions. The XbaI may be heat inactivated. The linearized plasmid was purified from enzyme and buffer salts and analyzed by agarose gel to confirm linearization. The IVT reaction to generate Cas9 modified mRNA was incubated at 37° C. for 2-4 hours in the following conditions: 50 ng/μL linearized plasmid; 2 mM each of GTP, ATP, CTP, and N1-methyl pseudo-UTP (Trilink); 10 mM ARCA (Trilink); 5 U/μL T7 RNA polymerase (NEB); 1 U/μL Murine RNase inhibitor (NEB); 0.004 U/μL inorganic E. coli pyrophosphatase (NEB); and 1× reaction buffer. After the 4-hour incubation, TURBO DNase (ThermoFisher) was added to a final concentration of 0.01 U/μL, and the reaction was incubated for an additional 30 minutes to remove the DNA template. The Cas9 mRNA was purified from enzyme and nucleotides using a MegaClear Transcription Clean-up kit per the manufacturer's protocol (ThermoFisher). Alternatively, the mRNA was purified through a precipitation protocol, which in some cases was followed by HPLC-based purification. Briefly, after the DNase digestion, the mRNA was precipitated by adding 0.21× volume of a 7.5 M LiCl solution and mixing, and the precipitated mRNA was pelleted by centrifugation. Once the supernatant was removed, the mRNA was reconstituted in water. The mRNA was precipitated again using ammonium acetate and ethanol. 5M Ammonium acetate was added to the mRNA solution for a final concentration of 2M along with 2× volume of 100% EtOH. The solution was mixed and incubated at −20° C. for 15 min. The precipitated mRNA was again pelleted by centrifugation, the supernatant was removed, and the mRNA was reconstituted in water. As a final step, the mRNA was precipitated using sodium acetate and ethanol. 1/10 volume of 3 M sodium acetate (pH 5.5) was added to the solution along with 2× volume of 100% EtOH. The solution was mixed and incubated at −20° C. for 15 min. The precipitated mRNA was again pelleted by centrifugation, the supernatant was removed, the pellet was washed with 70% cold ethanol and allowed to air dry. The mRNA was reconstituted in water. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. The transcript concentration was determined by measuring the light absorbance at 260 nm (Nanodrop), and the transcript was analyzed by capillary electrophoresis by Bioanalyzer (Agilent).

Cas9 mRNA and gRNA Transfections in Neuro2A Cells

The mouse cell line Neuro2A was cultured in DMEM media supplemented with 10% fetal bovine serum and was plated at a density of 15,000 cells/well in a 96-well plate 24 hours prior to transfection. On the day of transfection, the media was aspirated from cells and replaced with fresh media. Lipofectamine-2000 (Invitrogen) was diluted 1:50 (v/v) in Opti-MEM (Invitrogen). Cas9 mRNA and guide RNA were diluted separately in Opti-MEM. Both Cas9 mRNA and gRNA were mixed separately 1:1 (v/v) with diluted Lipofectamine-2000, producing two lipoplexes. After 5 minutes of incubation, lipoplexes were added in succession to cells, for a final concentration of 100 ng Cas9 mRNA and 0.4 μL total lipofection reagent per well. Guides were tested at four dose levels, including 3 nM, 0.3 nM, 0.03 nM, and 0.003 nM. Cells were lysed 24 hours post transfection, and lysates were used directly in the PCR reaction that was analyzed for editing by NGS.

### Primary Hepatocytes

Primary human liver hepatocytes (PHH), primary cynomolgus liver hepatocytes (PCH), or primary mouse liver hepatocytes (PMH) (Thermo Fisher) were cultured per the manufacturer's protocol (Invitrogen, protocol 11.28.2012). In brief, the cells were thawed and resuspended in hepatocyte thawing medium (Thermo Fisher, Cat. CM7000) followed by centrifugation at 100 g for 10 minutes for PHH, 100 g for 4 min for PMH, or 80 g for 4 minutes for PCH. The supernatant was discarded and the pelleted cells resuspended in hepatocyte plating medium plus supplement pack (Invitrogen, Cat. A1217601 and CM3000). Cells were counted and plated on Bio-coat collagen I coated 96-well plates (Thermo Fisher, Cat. 877272) at a density of 30,000-35,000 cells/well for PHH, 50,000-60,000 cells/well for PCH, or 15,000-20,000 cells/well for PMH. Plated cells were allowed to settle and adhere for 4 to 6 hours in a tissue culture incubator at 37° C. and 5% CO₂ atmosphere. After incubation, cells were checked for monolayer formation. Cells were then washed with hepatocyte maintenance media/culture media with serum-free supplement pack (Invitrogen, Cat. A1217601 and CM4000) and then fresh hepatocyte maintenance media was added on to the cells

For lipoplex transfection experiments, Lipofectamine RNAiMax (ThermoFisher, Cat. 13778150) based transfections were conducted as per the manufacturer's protocol. Cells were transfected with a single lipoplex containing Spy Cas9 mRNA (100 ng for PMH, 50 ng for PHH and 25 ng for PCH) and OptiMem sgRNA (25 nM for PMH, 12.5 nM for PHH and 0.125 nM for PCH) and OptiMem and Lipofectamine RNAiMax (1 μL/well for PHH and PCH both and 2 uL/well for PMH).

For experiments involving LNP treatment, after 4-6 hours, the plating media was removed, cells were then washed with hepatocyte maintenance media/culture media with serum-free supplement pack (Invitrogen, Cat. A1217601 and CM4000), and replaced with supplemented hepatocyte culture medium (Invitrogen, Cat. A1217601 and CM4000) containing LNP formulated Cas9 mRNA and guide RNA plus 3% serum. LNPs were diluted from a starting dose level of 100 ng Cas9 mRNA and approximately 30 nM guide RNA per well, carrying out serial dilutions down to 0.1 ng mRNA and 0.03 nM guide per well. Cells were incubated for approximately 72 hours at 37° C. and 5% CO₂ atmosphere before cell lysis and NGS analysis as described herein.

### HepG2

The human hepatocellular carcinoma cell line HepG2 (American Type Culture Collection, Cat. HB-8065) was cultured in DMEM media containing penstrep supplemented with 10% fetal bovine serum. Cells were counted and plated on Bio-coat collagen I coated 96-well plates (ThermoFisher, Cat. 877272) at a density of 10,000 cells/well in a 96-well plate 24 hours prior to transfection.

After 4-6 hours, the plating media was removed, cells were then washed with hepatocyte maintenance media/culture media with serum-free supplement pack (Invitrogen, Cat. A1217601 and CM4000), and replaced with supplemented hepatocyte culture medium (Invitrogen, Cat. A1217601 and CM4000) containing LNP formulated Cas9 mRNA and guide RNA plus 3% serum. LNPs were diluted from a starting dose level of 100 ng Cas9 mRNA and approximately 30 nM guide RNA per well, carrying out serial dilutions down to 0.1 ng mRNA and 0.03 nM guide per well. Cells were incubated for approximately 72 hours at 37° C. and 5% CO₂ atmosphere before cell lysis and NGS analysis as described herein.

### Lipid Nanoparticle (“LNP”) Formulation

LNP Procedure A: LNPs were formulated with a cationic lipid amine to RNA phosphate (N:P) molar ratio of about 4.5. The lipid nanoparticle components were dissolved in 100% ethanol with the following molar ratios: 45 mol-% (12.7 mM) cationic lipid (e.g., (9Z,12Z)-3-((4,4-bis(octyloxy)butanoyl)oxy)-2-((((3-(diethylamino)propoxy)-carbonyl)oxy)methyl)propyl octadeca-9,12-dienoate, also called 3-((4,4-bis(octyloxy)-butanoyl)oxy)-2-((((3-(diethylamino)propoxy)carbonyl)oxy)methyl)propyl (9Z,12Z)-octadeca-9,12-dienoate); 44 mol-% (12.4 mM) helper lipid (e.g., cholesterol); 9 mol-% (2.53 mM) neutral lipid (e.g., DSPC); and 2 mol-% (0.563 mM) PEG (e.g., PEG2k-DMG).

The LNPs were formed by microfluidic mixing of the lipid and RNA solutions using a Precision Nanosystems NanoAssemblr™ Benchtop Instrument, according to the manufacturer's protocol. A 2:1 ratio of aqueous to organic solvent was maintained during mixing using differential flow rates.

The RNA cargo were prepared in 25 mM sodium acetate buffer, pH 4.5, resulting in a concentration of RNA cargo of approximately 0.45 mg/mL, with a ratio of Cas9 mRNA:sgRNA of 1:1 (wt/wt). After mixing, the LNPs were collected, diluted in 50 mM Tris at pH 7.5 (approximately 1:1), and then LNPs were exchanged into 50 mM Tris at pH 7.5 (100-fold excess of sample volume), overnight at 4° C. under gentle stirring using a 10 kDa Slide-a-Lyzer™ G2 Dialysis Cassette (ThermoFisher Scientific). The LNPs were concentrated using 10 kDa Amicon spin filter (centrifugation at 4000 g at 4° C.) to achieve twice the desired concentration. These concentrated LNPs were mixed 1:1 with 50 mM Tris, 90 mM NaCl, 10% sucrose at pH 7.5 (2× TSS). The resulting mixture was then filtered using a 0.2 μM sterile filter. The resulting filtrate was stored at 2-8° C.

LNP Procedure B: LNPs were formulated with a cationic lipid amine to RNA phosphate (N:P) molar ratio of about 4.5. The lipid nanoparticle components were dissolved in 100% ethanol with the following molar ratios: 45 mol-% (12.7 mM) cationic lipid (e.g., (9Z,12Z)-3-((4,4-bis(octyloxy)butanoyl)oxy)-2-((((3-(diethylamino)propoxy)-carbonyl)oxy)methyl)propyl octadeca-9,12-dienoate, also called 3-((((4-bis(octyloxy)-butanoyl)oxy)-2-((((3-(diethylamino)propoxy)carbonyl)oxy)methyl)propyl (9Z,12Z)-octadeca-9,12-dienoate); 44 mol-% (12.4 mM) helper lipid (e.g., cholesterol); 9 mol-% (2.53 mM) neutral lipid (e.g., DSPC); and 2 mol-% (0.563 mM) PEG (e.g., PEG2k-DMG).

The LNPs were formed by microfluidic mixing of the lipid and RNA solutions using a Precision Nanosystems NanoAssemblr™ Benchtop Instrument, according to the manufacturer's protocol. A 2:1 ratio of aqueous to organic solvent was maintained during mixing using differential flow rates.

The RNA cargo were prepared in 25 mM sodium citrate, 100 mM sodium chloride at pH 5 resulting in a concentration of RNA cargo of approximately 0.45 mg/mL. After mixing, the LNPs were collected in water at the ratio of 3:1. The LNPs were incubated for an hour at room temperature and mixed 1:1 with water. Then they were buffer-exchanged into 1× TSS (50 mM Tris, 45 mM NaCl, 5% sucrose at pH 7.5) on PD-10 columns (GE Healthcare), using manufacturer's protocol. The LNPs were concentrated using 10 kDa Amicon spin filter (centrifugation at 4000 g at 4° C.) to achieve the desired concentration. The resulting mixture was then filtered using a 0.2 μm sterile filter. The resulting filtrate was stored at −80° C.

LNP Procedure C: LNPs were formulated with a cationic lipid amine to RNA phosphate (N:P) molar ratio of about 6. The lipid nanoparticle components were dissolved in 100% ethanol with the following molar ratios: 50 mol-% (12.7 mM) cationic lipid (e.g., (9Z,12Z)-3-((4,4-bis(octyloxy)butanoyl)oxy)-2-((((3-(diethylamino)propoxy)-carbonyl)oxy)methyl)propyl octadeca-9,12-dienoate, also called 3-((4,4-bis(octyloxy)-butanoyl)oxy)-2-((((3-(diethylamino)propoxy)carbonyl)oxy)methyl)propyl (9Z,12Z)-octadeca-9,12-dienoate); 38 mol-% (12.4 mM) helper lipid (e.g., cholesterol); 9 mol-% (2.53 mM) neutral lipid (e.g., DSPC); and 3 mol-% (0.563 mM) PEG (e.g., PEG2k-DMG).

The LNPs were formed by microfluidic mixing of the lipid and RNA solutions using a Precision Nanosystems NanoAssemblr™ Benchtop Instrument, according to the manufacturer's protocol. A 2:1 ratio of aqueous to organic solvent was maintained during mixing using differential flow rates.

The RNA cargo was prepared in 25 mM sodium citrate, 100 mM sodium chloride at pH 5 resulting in a concentration of RNA cargo of approximately 0.45 mg/mL. LNPs were formed by an impinging jet mixing method where one stream of lipids in ethanol were mixed with two streams of RNA in citrate buffer through a 0.25 mm ID Cross piece. The two RNA streams mix perpendicular to the ethanol stream. A fourth stream of water for injection (WFI) meets the resulting particles in an in-line dilution through a 0.5 mm ID Tee piece. All four streams are delivered at 10 mL/min using a syringe pump. These LNPs were incubated for an hour at room temperature and then they were buffer-exchanged into 1× TSS (50 mM Tris, 45 mM NaCl, 5% sucrose at pH 7.5) on PD-10 columns (GE Healthcare), using manufacturer's protocol. The LNPs were concentrated using 10 kDa Amicon spin filter (centrifugation at 4000 g at 4° C.) to achieve the desired concentration. The resulting mixture was then filtered using a 0.2 μm sterile filter. The resulting filtrate was stored at −80° C.

LNP Procedure D: LNPs were formulated with a cationic lipid amine to RNA phosphate (N:P) molar ratio of about 4.5. The lipid nanoparticle components were dissolved in 100% ethanol with the following molar ratios: 45 mol-% (12.7 mM) cationic lipid (e.g., (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate); 44 mol-% (12.4 mM) helper lipid (e.g., cholesterol); 9 mol-% (2.53 mM) neutral lipid (e.g., DSPC); and 2 mol-% (0.563 mM) PEG (e.g., PEG2k-DMG). The RNA cargo were prepared in 25 mM sodium acetate buffer, pH 4.5, resulting in a concentration of RNA cargo of approximately 0.45 mg/mL, with a ratio of Cas9 mRNA: sgRNA of 1:1 (wt/wt).

LNPs were prepared using a cross-flow technique by impinging jet mixing of the lipid in ethanol with two volumes of RNA solutions and one volume of water. The lipid in ethanol is mixed through a mixing cross with the two volumes of RNA solution. A fourth stream of water is mixed with the outlet stream of the cross through an inline tee. (See WO2016010840 FIG. 2.) The LNPs were held for 1 hour at room temperature, and further diluted with water (approximately 1:1 v/v). Diluted LNPs were concentrated using tangential flow filtration on a flat sheet cartridge (Sartorius, 100 kD MWCO) and then buffer exchanged by diafiltration into 50 mM Tris, 45 mM NaCl, 5% (w/v) sucrose, pH 7.5 (TSS). Alternatively, the final buffer exchange into TSS was completed with PD-10 desalting columns (GE). If required, formulations were concentrated by centrifugation with Amicon 100 kDa centrifugal filters (Millipore). The resulting mixture was then filtered using a 0.2 μm sterile filter. The final LNP was stored at 4° C. or -80° C. until further use.

### Next-Generation Sequencing (“NGS”) and Analysis for On-Target Cleavage Efficiency

To quantitatively determine the efficiency of editing at the target location in the genome, deep sequencing was utilized to identify the presence of insertions and deletions introduced by gene editing.

PCR primers were designed around the target site (e.g., within the target gene of interest (e.g., TTR)), and the genomic area of interest was amplified.

Additional PCR was performed according to the manufacturer's protocols (Illumina) to add the necessary chemistry for sequencing. The amplicons were sequenced on an Illumina MiSeq instrument. The reads were aligned to the human reference genome (e.g., hg38) after eliminating those having low quality scores. The resulting files containing the reads were mapped to the reference genome (BAM files), where reads that overlapped the target region of interest were selected and the number of wild type reads versus the number of reads which contain an insertion, substitution, or deletion was calculated.

The editing percentage (e.g., the “editing efficiency” or “percent editing”) is defined as the total number of sequence reads with insertions or deletions over the total number of sequence reads, including wild type.

### LNP Delivery In Vivo

CD-1 female mice, ranging 6-10 weeks of age were used in each study involving mice. Sprague-Dawley female rats, ranging 6-10 weeks of age were used in each study involving rats. Animals were weighed and grouped according to body weight for preparing dosing solutions based on group average weight. LNPs were dosed via the lateral tail vein in a volume of 0.2 mL per animal (approximately 10 mL per kilogram body weight). The animals were observed at approximately 6 hours post dose for adverse effects. Body weight was measured at twenty-four hours post-administration, and animals were euthanized at various time points by exsanguination via cardiac puncture under isoflourane anesthesia. Blood was collected into serum separator tubes or into tubes containing buffered sodium citrate for plasma as described herein. For studies involving in vivo editing, liver tissue was collected from the median lobe from each animal for DNA extraction and analysis.

### Genomic DNA Isolation

For the in vivo studies, genomic DNA was extracted from 10 mg of tissue using a bead-based extraction kit, MagMAX-96 DNA Multi-Sample Kit (ThermoFisher, Cat #4413020) according to the manufacturer's protocol, which includes homogenizing the tissue in lysis buffer (approximately 400 μL/10 mg tissue). All DNA samples were normalized to 100 ng/μL concentration for PCR and subsequent NGS analysis, as described herein.

### Transthyretin (TTR) ELISA Analysis Used in Animal Studies

Blood was collected, and the serum was isolated as indicated. The total TTR serum levels were determined using a Mouse Prealbumin (Transthyretin) ELISA Kit (Aviva Systems Biology, Cat. OKIA00111); rat TTR serum levels were measured using a rat specific ELISA kit (Aviva Systems Biology catalog number OKIA00159). Kit reagents and standards were prepared according to the manufacturer's protocol. Mouse serum was diluted to a final dilution of 10,000-fold with 1× assay diluent. This was done by carrying out two sequential 50-fold dilutions resulting in a 2500-fold dilution. A final 4-fold dilution step was carried out for a total sample dilution of 10,000-fold. Both standard curve dilutions (100 μL each) and diluted serum samples were added to each well of the ELISA plate pre-coated with capture antibody. The plate was incubated at room temperature for 30 minutes before washing. Enzyme-antibody conjugate (100 μL per well) was added for a 20-minute incubation. Unbound antibody conjugate was removed and the plate was washed again before the addition of the chromogenic substrate solution. The plate was incubated for 10 minutes before adding 100 μL of the stop solution, e.g., sulfuric acid (approximately 0.3 M). The plate was read on a SpectraMax M5 plate reader at an absorbance of 450 nm. Serum TTR levels were calculated by SoftMax Pro software ver. 6.4.2 using a four parameter logistic curve fit off the standard curve. Final serum values were adjusted for the assay dilution. Percent knockdown (% KD) values were determined relative to controls, which generally were animals sham-treated with vehicle (transport and storage solution or TSS) unless otherwise indicated.

### Nuclease Susceptibility Assays

Assays to determine and quantify where sgRNA cleavage occurs upon exposure to hepatocyte cytosol and to assess the effect of sgRNA modifications on stability were performed as follows. sgRNAs at 15 μM were incubated with human liver cytosol (XenoTech Product H0610.C) (adjusted to 0.01 mg/mL final protein concentration using pH 7.4 phosphate-buffered saline unless otherwise indicated) for a time period as indicated below. Reactions were stopped by adding 67 μL of proteinase K cell lysis buffer solution, which consisted of 3.230 mL water, 2.125 mL of tissue and cell lysis solution (Epicentre Product MTC096H), and 340 μL of proteinase K (50 mg/mL from Epicentre Product MPRK092) and incubating for 30 minutes at 65° C. in a thermo-mixer shaking at 750 RPM. 8 μL of 3M KCl was then added and the mixture was incubated for 10 minutes at 0° C. The mixtures were then centrifuged for 15 minutes at 1500 g to precipitate detergent. The supernatant was removed, diluted with 0.95 mL of dilution buffer (consisting of 0.01% Tween20 in water), and mixed with 1 mL of pH 4.3 loading/dilution buffer (consisting of 10 mM sodium acetate, 10% acetonitrile, 0.01% Tween 20, 10 mM EDTA, and 1 mM TCEP) and the mixture was loaded on a Clarity® OTX™ SPE oligonucleotide purification cartridge. Washes were performed at pH 4.3, 5.5, and about 7, followed by elution at pH 9.0. The eluate was dried under vacuum and resuspended in 100 mM triethyl ammonium acetate (TEAAc).

Samples were then analyzed by LC/MS.

### Example 2—Modified Dual Guide Survey in HEK293 Cells

A survey of chemical modification within crRNA was performed to identify negative influence of chemical modification at specific positions on editing efficacy. Each crRNA in this survey targeted the identical sequence within the human BCL11A gene. Test guides contained modifications within the spacer region of the crRNA (positions 1-20 from the 5′ end) limited to either a single modified base or two adjacent bases with the same chemical modification as described in Table 6. Phosophothioate bonds (PS), inosine substitution, DNA bases, 2′OMe modifications, and unlocked nucleic acids (UNA) were assayed.

The human embryonic kidney adenocarcinoma cell line HEK293 constitutively expressing Spy Cas9 (“HEK293_Cas9”) was cultured in DMEM media supplemented with 10% fetal bovine serum. Cells were plated at a density of 15,000 cells/well in a 96-well plate 20 hours prior to transfection (˜70% confluent at time of transfection). Cells were transfected with Lipofectamine RNAiMAX (ThermoFisher, Cat. 13778150) according to the manufacturer's protocol. Cells were transfected with a lipoplex containing individual crRNA (3.1 nM), trRNA TR000002 (3.1 nM), Lipofectamine RNAiMAX (0.45 μL/well) and OptiMem. Cells were lysed 48 hours post transfection, and lysates were used directly in the PCR reaction that was analyzed for editing by NGS.

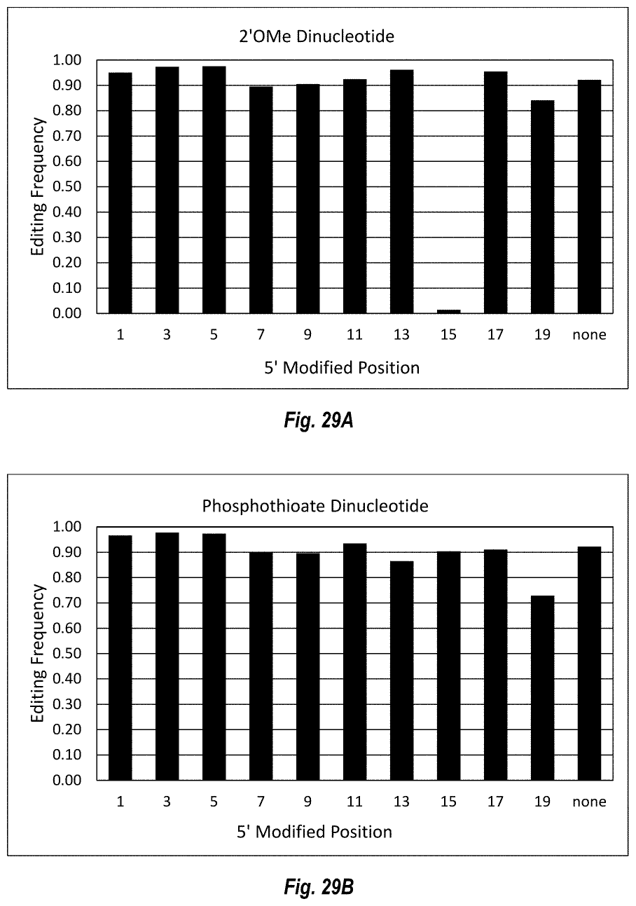

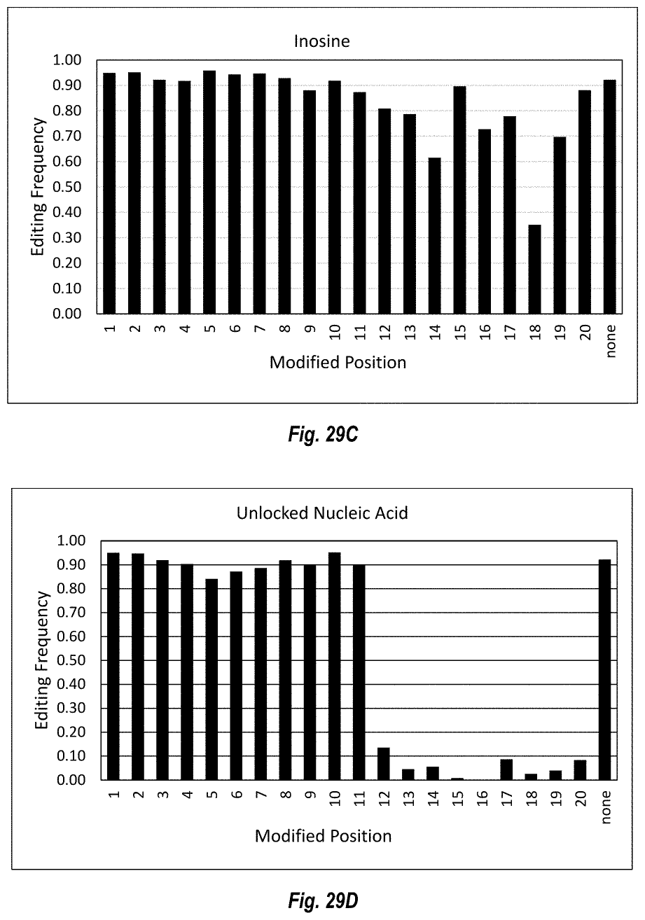

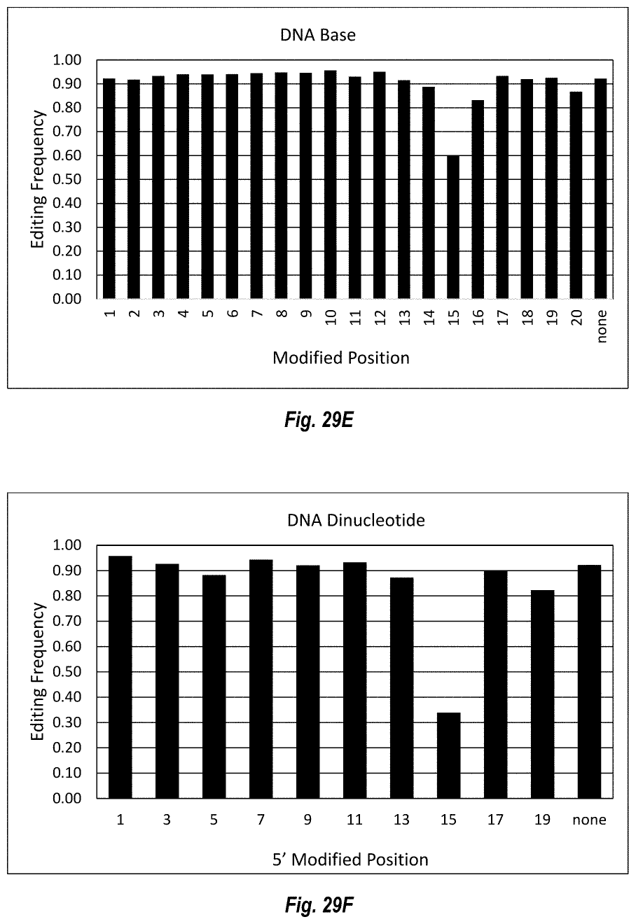

The editing results are shown in Table 6 and FIGS. 29A-F. Editing efficiency was reduced to near background levels by 2′OMe modification at positions 15 and 16 (FIG. 29A). Phosphorothioate dinucleotides reduced editing by roughly 20% when placed at positions 19 and 20 of the spacer region (FIG. 29B). Inosine substitution in the seed region led to moderate negative impact on editing, for instance a roughly 30% decrease when modified at position 14 or a roughly 60% decrease at position 18 (FIG. 29C). UNA base substitution at any single position from position 11 to position 20 severely reduced editing efficacy (FIG. 29D). DNA base substitution at position 15 reduced editing efficiency by about one third (FIG. 29E). DNA base substitution at both positions 15 and 16 reduced editing by about two thirds (FIG. 29F).

TABLE 6
Survey of Chemical Modification in crRNAs

| Modification | | Editing | | Modification | | Editing | |
|---|---|---|---|---|---|---|---|
| & Position | Guide | % | SD | & Position | Guide | % | SD |
| No crRNA | n/a | 0.2% | 0.1% | Inosine 5 | CR007559 | 95.8% | 0.9% |
| Unmodified | CR000309 | 92.2% | 5.9% | Inosine 6 | CR007560 | 94.3% | 1.5% |
| (none) | | | | | | | |
| 2′OMe 1, 2 | CR005084 | 95.0% | 0.5% | Inosine 7 | CR007561 | 94.7% | 0.4% |
| 2′OMe 3, 4 | CR005085 | 97.3% | 1.3% | Inosine 8 | CR007562 | 92.8% | 2.1% |
| 2′OMe 5, 6 | CR005086 | 97.6% | 0.5% | Inosine 9 | CR007563 | 88.1% | 4.2% |
| 2′OMe 7, 8 | CR005087 | 89.5% | 2.3% | Inosine 10 | CR007564 | 91.8% | 4.8% |
| 2′OMe 9, 10 | CR005088 | 90.5% | 1.4% | Inosine 11 | CR007565 | 87.3% | 4.9% |
| 2′OMe 11, 12 | CR005089 | 92.5% | 3.2% | Inosine 12 | CR007566 | 80.9% | 10.3% |
| 2′OMe 13, 14 | CR005090 | 96.2% | 0.4% | Inosine 13 | CR007567 | 78.7% | 5.4% |
| 2′OMe 15, 16 | CR005091 | 1.4% | 2.2% | Inosine 14 | CR007568 | 61.5% | 4.1% |
| 2′OMe 17, 18 | CR005092 | 95.5% | 1.1% | Inosine 15 | CR007569 | 89.6% | 5.3% |
| 2′OMe 19, 20 | CR005093 | 84.1% | 2.2% | Inosine 16 | CR007570 | 72.7% | 10.7% |
| PS 1, 2 | CR005100 | 96.6% | 1.4% | Inosine 17 | CR007571 | 77.9% | 6.1% |
| PS 3, 4 | CR005101 | 97.8% | 0.4% | Inosine 18 | CR007572 | 35.1% | 8.9% |
| PS 5, 6 | CR005102 | 97.3% | 0.2% | Inosine 19 | CR007573 | 69.7% | 3.2% |
| PS 7, 8 | CR005103 | 90.0% | 2.1% | Inosine 20 | CR007574 | 88.1% | 6.0% |
| PS 9, 10 | CR005104 | 89.7% | 0.9% | DNA 1 | CR007575 | 92.3% | 1.9% |
| PS 11, 12 | CR005105 | 93.5% | 1.2% | DNA 2 | CR007576 | 91.8% | 3.5% |
| PS 13, 14 | CR005106 | 86.5% | 1.5% | DNA 3 | CR007577 | 93.3% | 1.9% |
| PS 15, 16 | CR005107 | 90.4% | 5.7% | DNA 4 | CR007578 | 94.0% | 2.2% |
| PS 17, 18 | CR005108 | 91.1% | 1.6% | DNA 5 | CR007579 | 94.0% | 1.3% |
| PS 19, 20 | CR005109 | 72.9% | 0.8% | DNA 6 | CR007580 | 94.1% | 2.6% |
| UNA 1 | CR007531 | 95.1% | 2.2% | DNA 7 | CR007581 | 94.5% | 2.5% |
| UNA 2 | CR007532 | 94.8% | 2.6% | DNA 8 | CR007582 | 94.8% | 1.6% |
| UNA 3 | CR007533 | 91.9% | 4.4% | DNA 9 | CR007583 | 94.6% | 1.1% |
| UNA 4 | CR007534 | 90.4% | 5.2% | DNA 10 | CR007584 | 95.7% | 0.5% |
| UNA 5 | CR007535 | 84.1% | 8.5% | DNA 11 | CR007585 | 93.1% | 2.4% |
| UNA 6 | CR007536 | 87.2% | 7.9% | DNA 12 | CR007586 | 95.0% | 3.0% |
| UNA 7 | CR007537 | 88.7% | 6.0% | DNA 13 | CR007587 | 91.5% | 2.8% |
| UNA 8 | CR007538 | 91.9% | 3.7% | DNA 14 | CR007588 | 88.8% | 3.1% |
| UNA 9 | CR007539 | 90.0% | 7.9% | DNA 15 | CR007589 | 59.8% | 8.6% |
| UNA 10 | CR007540 | 95.2% | 2.2% | DNA 16 | CR007590 | 83.2% | 8.2% |
| UNA 11 | CR007541 | 90.0% | 4.1% | DNA 17 | CR007591 | 93.3% | 3.1% |
| UNA 12 | CR007542 | 13.6% | 9.8% | DNA 18 | CR007592 | 92.0% | 1.8% |
| UNA 13 | CR007543 | 4.6% | 5.9% | DNA 19 | CR007593 | 92.5% | 3.5% |
| UNA 14 | CR007544 | 5.6% | 5.3% | DNA 20 | CR007594 | 86.7% | 3.5% |
| UNA 15 | CR007545 | 0.8% | 1.2% | DNA 1, 2 | CR007595 | 95.8% | 1.9% |
| UNA 16 | CR007546 | 0.2% | 0.2% | DNA 3, 4 | CR007596 | 92.7% | 2.9% |
| UNA 17 | CR007547 | 8.7% | 5.2% | DNA 5, 6 | CR007597 | 88.3% | 8.4% |
| UNA 18 | CR007548 | 2.6% | 2.1% | DNA 7, 8 | CR007598 | 94.4% | 2.4% |
| UNA 19 | CR007549 | 4.0% | 1.8% | DNA 9, 10 | CR007599 | 92.1% | 2.2% |
| UNA 20 | CR007550 | 8.3% | 5.1% | DNA 11, 12 | CR007600 | 93.3% | 2.8% |
| Inosine 1 | CR007555 | 94.9% | 4.3% | DNA 13, 14 | CR007601 | 87.3% | 1.3% |
| Inosine 2 | CR007556 | 95.1% | 0.2% | DNA 15, 16 | CR007602 | 33.9% | 1.6% |
| Inosine 3 | CR007557 | 92.2% | 3.1% | DNA 17, 18 | CR007603 | 90.2% | 3.4% |
| Inosine 4 | CR007558 | 91.8% | 2.9% | DNA 19, 20 | CR007604 | 82.3% | 3.6% |

### Example 3—Representative Dual Guide Screen

The impacts of chemical modification type and position were evaluated in an editing screen of modified crRNAs. The screen assayed guides modified with 2′F, 2′OMe and PS. The complete pattern set was applied to guides targeting six distinct sites in the TTR gene. The final data set contained 1704 distinct guides and 284 unique modification patterns.

Guide modification patterns were computationally selected to minimize the number of modification combinations required to explore the large combinatorial space of possible modification patterns. Patterns were chosen to create a uniform distribution of modifications at each individual position and also at each pair of positions, so that no particular position or combination of positions was over-represented in the final set. This bias-minimization approach allowed for testing of individual positional effects as well as for detection of higher-order interaction effects between positions. Appropriate controls were added to the set to control for nuisance effects such as guide domain sequence, transfection efficiency and other experimental variability.

Each pattern in this set contained only one type of modification on positions 4 to 20; modification types were not mixed within patterns. The final set of patterns consisted of 3 groups of 96 patterns on positions 4 to 20 having 0-4 2′F modifications, 0-4 2′Ome modifications or 0-15 PS modifications. PS modifications were more heavily applied because previous observations indicated that the PS modification is better tolerated than 2′Flu or 2′Ome, and therefore less likely to show detectable effects when present in small numbers.

The human embryonic kidney adenocarcinoma cell line HEK293 constitutively expressing Spy Cas9 (“HEK293_Cas9”) was cultured in DMEM media supplemented with 10% fetal bovine serum. Cells were plated at a density of 10,000 cells/well in a 96-well plate about 24 hours prior to transfection (˜70% confluent at time of transfection). Cells were transfected with Lipofectamine RNAiMAX (ThermoFisher, Cat. 13778150) according to the manufacturer's protocol. Cells were transfected with a lipoplex containing individual crRNA (25 nM), trRNA TR009880 (25 nM), Lipofectamine RNAiMAX (0.3 μL/well) and OptiMem. Cells were lysed 48 hours post transfection, and lysates were used directly in the PCR reaction that was analyzed for editing by NGS.

The editing results are described in Table 8. Each row number represents a single modification pattern. The first two rows in the table show controls.

The overall impact of the modification type, targeting location, and guide domain sequence were evaluated in the modified guides. The data show a wide range of activity within the modified oligonucleotides, from close to 0 to nearly 90% editing. In general, the 2′F modifications were more well-tolerated than 2′OMe or PS modified guides, though the PS guides were more heavily modified, on average, than the other modifications. We observed a clear impact of guide domain's nucleobase sequence on response to modification. G480 and G490 variants were strongly impaired by 2′OMe modifications, but were resistant to 2′F and PS modifications, whereas G494, G499 and G502 variants were most strongly impacted by PS modifications, and less so by 2′OMe, and G488 responded similarly to all three modifications.

A regression-based analysis was conducted to identify the modification types with significant impact on guide activity. The editing data were first corrected for guide sequence and plate effects prior to modeling. Positional modification impacts were then modeled as independent linearly additive factors using standard regression techniques. A separate analysis examined whether there was evidence of interaction and non-linear relationships among positions. No significant higher-order effects were observed; the results reported below come from the initial linear regression analysis. The editing data for all the modified guides were corrected for guide sequence effects and then modeled for modification impact.

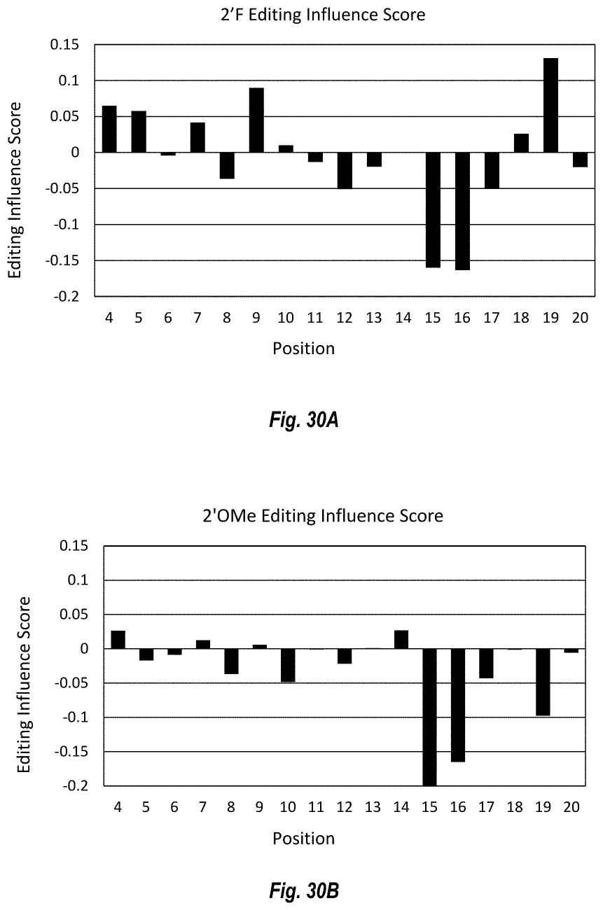

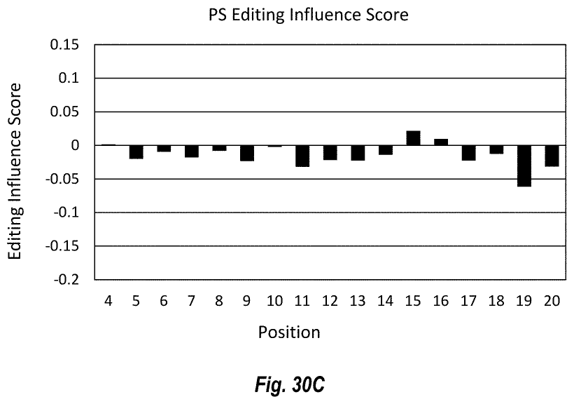

2′ modifications at a number of positions displayed negative impacts on editing as shown in Table 7 and FIG. 30A-C. For example, 2′F or 2′OMe modification of positions 15 or 16 resulted in statistically significant inhibition of editing activity, suggesting that gRNAs with ribose at positions 15 and 16 are preferred. In contrast, 2′F modification of position 19 significantly increased editing. The regression model found that this modification added an additional 13% editing over baseline, on average. 2′OMe had the opposite effect, strongly inhibiting editing. No other positions were consistently significantly impactful, although individual sequences were in many cases affected. PS modifications had a possible small negative effect at some positions, including position 19. Highly modified gRNA performed worse than less modified gRNA, indicating that the amount of PS modification may be relevant.

TABLE 7
Editing Influence Scores

| | 2′F | | | 2′Ome | | | PS | | |
|---|---|---|---|---|---|---|---|---|---|
| Position | Score | SE | P value | Score | SE | P value | Score | SE | P value |
| 4 | 0.065 | 0.045 | 0.151 | 0.027 | 0.015 | 0.084 | 0.002 | 0.011 | 0.885 |
| 5 | 0.058 | 0.044 | 0.186 | −0.017 | 0.015 | 0.249 | −0.020 | 0.010 | 0.054 |
| 6 | −0.004 | 0.044 | 0.923 | −0.009 | 0.015 | 0.555 | −0.009 | 0.012 | 0.438 |
| 7 | 0.042 | 0.044 | 0.347 | 0.013 | 0.015 | 0.404 | −0.018 | 0.011 | 0.100 |
| 8 | −0.037 | 0.046 | 0.431 | −0.037 | 0.016 | 0.020 | −0.008 | 0.011 | 0.446 |
| 9 | 0.090 | 0.044 | 0.040 | 0.006 | 0.015 | 0.690 | −0.023 | 0.010 | 0.025 |
| 10 | 0.010 | 0.045 | 0.819 | −0.048 | 0.015 | 0.002 | −0.002 | 0.011 | 0.821 |
| 11 | −0.013 | 0.046 | 0.773 | −0.001 | 0.015 | 0.947 | −0.032 | 0.012 | 0.006 |
| 12 | −0.051 | 0.045 | 0.262 | −0.022 | 0.015 | 0.158 | −0.022 | 0.011 | 0.049 |
| 13 | −0.020 | 0.045 | 0.662 | 0.001 | 0.015 | 0.937 | −0.023 | 0.011 | 0.039 |
| 14 | 0.000 | 0.045 | 0.995 | 0.027 | 0.015 | 0.078 | −0.014 | 0.010 | 0.178 |
| 15 | −0.160 | 0.044 | <4E−4 | −0.201 | 0.015 | <2E−16 | 0.022 | 0.012 | 0.067 |
| 16 | −0.164 | 0.045 | <3E−4 | −0.165 | 0.015 | <2E−16 | 0.010 | 0.011 | 0.378 |
| 17 | −0.050 | 0.045 | 0.266 | −0.043 | 0.015 | 0.005 | −0.023 | 0.011 | 0.047 |
| 18 | 0.026 | 0.043 | 0.545 | −0.001 | 0.015 | 0.930 | −0.013 | 0.011 | 0.259 |
| 19 | 0.131 | 0.044 | 0.003 | −0.098 | 0.015 | <2E−10 | −0.062 | 0.011 | <3E−8 |
| 20 | −0.020 | 0.044 | 0.649 | −0.006 | 0.015 | 0.694 | −0.032 | 0.011 | 0.003 |

TABLE 8
Editing in HEKCas9 cells with modified dgRNA

| G480 Var | Edit | SD | G488 Var | Edit | SD | G490 Var | Edit | SD |
|---|---|---|---|---|---|---|---|---|
| CR09711 | 51.3 | 10.8 | CR10224 | 9.2 | 5.5 | CR10319 | 40.3 | 7.7 |
| CR03353 | 57.5 | 13.9 | CR03346 | 14.2 | 10.3 | CR03366 | 49.3 | 7.7 |
| CR10040 | 70.9 | 13.6 | CR10130 | 16.7 | 2.9 | CR10225 | 74.5 | 6.3 |
| CR10041 | 70.4 | 7.0 | CR10131 | 19.4 | 3.1 | CR10226 | 50.2 | 10.7 |
| CR10042 | 41.9 | 36.5 | CR10132 | 3.8 | 0.6 | CR10227 | 38.4 | 3.8 |
| CR10043 | 72.5 | 6.8 | CR10133 | 14.1 | 2.4 | CR10228 | 61.5 | 2.8 |
| CR10044 | 61.9 | 11.7 | CR10134 | 4.1 | 1.1 | CR10229 | 52.5 | 4.9 |
| CR10045 | 70.0 | 6.7 | CR10135 | 14.0 | 4.6 | CR10230 | 49.7 | 3.7 |
| CR10046 | 79.3 | 6.9 | CR10136 | 9.1 | 1.4 | CR10231 | 53.9 | 3.9 |
| CR10047 | 63.6 | 7.1 | CR10137 | 11.2 | 2.4 | CR10232 | 55.6 | 1.9 |
| CR10048 | 77.1 | 6.2 | CR10138 | 18.3 | 0.8 | CR10233 | 44.6 | 4.2 |
| CR10049 | 71.5 | 8.2 | CR10139 | 15.3 | 0.7 | CR10234 | 37.4 | 2.8 |
| CR10050 | 77.0 | 6.7 | CR10140 | 11.8 | 1.8 | CR10235 | 57.3 | 2.5 |
| CR10051 | 65.6 | 6.1 | CR10141 | 5.5 | 1.1 | CR10236 | 25.8 | 4.7 |
| CR10052 | 65.4 | 4.1 | CR10142 | 5.4 | 1.5 | CR10237 | 25.1 | 2.6 |
| CR10053 | 58.5 | 17.0 | CR10143 | 5.5 | 0.6 | CR10238 | 33.0 | 5.0 |
| CR10054 | 59.0 | 18.4 | CR10144 | 4.2 | 1.2 | CR10239 | 29.7 | 3.6 |
| CR10055 | 50.0 | 14.7 | CR10145 | 5.7 | 2.1 | CR10240 | 27.6 | 5.4 |
| CR10056 | 50.0 | 18.2 | CR10146 | 7.4 | 1.7 | CR10241 | 37.2 | 8.0 |
| CR10057 | 47.2 | 18.0 | CR10147 | 3.9 | 0.2 | CR10242 | 29.4 | 5.6 |
| CR10058 | 37.9 | 18.0 | CR10148 | 2.4 | 1.0 | CR10243 | 17.1 | 1.8 |
| CR10059 | 66.4 | 14.4 | CR10149 | 9.1 | 1.4 | CR10244 | 22.3 | 3.5 |
| CR10060 | 76.6 | 10.2 | CR10150 | 13.6 | 3.9 | CR10245 | 55.1 | 11.0 |
| CR10061 | 63.1 | 5.9 | CR10151 | 3.1 | 1.0 | CR10246 | 45.6 | 5.9 |
| CR10062 | 60.6 | 18.6 | CR10152 | 9.6 | 2.8 | CR10247 | 31.2 | 4.7 |
| CR10063 | 47.0 | 15.2 | CR10153 | 4.7 | 0.5 | CR10248 | 31.4 | 9.3 |
| CR10064 | 46.0 | 21.0 | CR10154 | 2.4 | 0.3 | CR10249 | 25.9 | 1.0 |
| CR10065 | 51.0 | 11.0 | CR10155 | 3.6 | 0.6 | CR10250 | 27.1 | 4.4 |
| CR10066 | 48.0 | 10.5 | CR10156 | 4.9 | 0.9 | CR10251 | 24.2 | 3.8 |
| CR10067 | 45.9 | 14.7 | CR10157 | 3.5 | 1.0 | CR10252 | 15.2 | 1.5 |
| CR09753 | 48.7 | 15.0 | CR10158 | 7.1 | 0.9 | CR10253 | 14.3 | 2.1 |
| CR10068 | 39.0 | 13.6 | CR10159 | 2.5 | 0.1 | CR10254 | 17.8 | 2.4 |
| CR10069 | 57.3 | 17.3 | CR10160 | 6.8 | 1.3 | CR10255 | 21.2 | 5.3 |
| CR10070 | 62.0 | 13.3 | CR10161 | 3.5 | 0.7 | CR10256 | 28.2 | 24.3 |
| CR10071 | 42.7 | 7.8 | CR10162 | 5.9 | 1.4 | CR10257 | 58.6 | 4.0 |
| CR10072 | 54.0 | 19.1 | CR10163 | 3.3 | 0.8 | CR10258 | 35.9 | 5.0 |
| CR10073 | 45.1 | 18.8 | CR10164 | 1.4 | 0.2 | CR10259 | 6.8 | 1.1 |
| CR10074 | 56.0 | 17.1 | CR10165 | 4.1 | 0.9 | CR10260 | 23.7 | 0.9 |
| CR10075 | 60.8 | 19.1 | CR10166 | 4.5 | 0.7 | CR10261 | 21.4 | 1.8 |
| CR10076 | 14.0 | 5.9 | CR10167 | 0.9 | 0.3 | CR10262 | 11.8 | 1.9 |
| CR10077 | 50.5 | 14.1 | CR10168 | 6.4 | 0.8 | CR10263 | 15.6 | 2.0 |
| CR10078 | 47.0 | 10.3 | CR10169 | 4.0 | 0.8 | CR10264 | 10.2 | 2.2 |
| CR10079 | 54.9 | 14.3 | CR10170 | 4.6 | 0.5 | CR10265 | 18.9 | 3.6 |
| CR10080 | 71.8 | 12.2 | CR10171 | 10.5 | 2.3 | CR10266 | 62.0 | 2.1 |
| CR10081 | 67.8 | 12.0 | CR10172 | 14.0 | 3.9 | CR10267 | 61.7 | 2.0 |
| CR10082 | 26.4 | 7.1 | CR10173 | 3.5 | 0.8 | CR10268 | 21.6 | 1.9 |
| CR10083 | 51.3 | 26.1 | CR10174 | 3.9 | 0.6 | CR10269 | 26.2 | 2.5 |
| CR10084 | 43.4 | 13.4 | CR10175 | 4.3 | 1.0 | CR10270 | 18.1 | 3.9 |
| CR10085 | 53.9 | 24.9 | CR10176 | 7.9 | 1.0 | CR10271 | 30.7 | 0.5 |
| CR10086 | 40.6 | 18.3 | CR10177 | 2.2 | 0.2 | CR10272 | 6.7 | 1.0 |
| CR10087 | 45.4 | 15.9 | CR10178 | 4.9 | 1.2 | CR10273 | 32.3 | 5.6 |
| CR10088 | 45.2 | 16.7 | CR10179 | 4.1 | 0.7 | CR10274 | 14.3 | 2.9 |
| CR10089 | 51.0 | 18.7 | CR10180 | 4.5 | 1.0 | CR10275 | 15.4 | 5.7 |
| CR10090 | 60.3 | 16.7 | CR10181 | 3.8 | 1.0 | CR10276 | 27.2 | 1.2 |
| CR10091 | 60.0 | 18.9 | CR10182 | 4.4 | 1.8 | CR10277 | 45.2 | 3.9 |
| CR10092 | 52.9 | 45.7 | CR10183 | 13.9 | 2.5 | CR10278 | 66.3 | 5.3 |
| CR10093 | 50.0 | 14.6 | CR10184 | 7.3 | 0.7 | CR10279 | 23.2 | 3.3 |
| CR10094 | 60.4 | 15.5 | CR10185 | 3.9 | 0.4 | CR10280 | 31.3 | 4.4 |
| CR10095 | 29.0 | 12.4 | CR10186 | 2.6 | 0.3 | CR10281 | 21.9 | 3.7 |
| CR10096 | 51.0 | 20.1 | CR10187 | 11.6 | 3.7 | CR10282 | 22.3 | 0.6 |
| CR10097 | 41.1 | 14.6 | CR10188 | 4.7 | 0.3 | CR10283 | 28.7 | 2.9 |
| CR10098 | 35.6 | 30.7 | CR10189 | 2.3 | 0.1 | CR10284 | 9.1 | 3.8 |
| CR10099 | 47.7 | 14.1 | CR10190 | 2.6 | 0.6 | CR10285 | 14.9 | 2.7 |
| CR10100 | 30.6 | 26.9 | CR10191 | 3.9 | 1.1 | CR10286 | 18.4 | 3.4 |
| CR10101 | 44.5 | 38.9 | CR10192 | 4.4 | 0.9 | CR10287 | 19.8 | 2.7 |
| CR10102 | 56.1 | 48.5 | CR10193 | 15.1 | 7.1 | CR10288 | 71.5 | 4.8 |
| CR10103 | 55.0 | 20.0 | CR10194 | 9.8 | 0.3 | CR10289 | 41.1 | 4.2 |
| CR10104 | 53.6 | 23.1 | CR10195 | 2.0 | 0.9 | CR10290 | 13.4 | 2.2 |
| CR10105 | 41.9 | 18.3 | CR10196 | 1.8 | 0.2 | CR10291 | 15.1 | 2.6 |
| CR10106 | 52.4 | 16.5 | CR10197 | 5.1 | 2.0 | CR10292 | 21.9 | 6.0 |
| CR10107 | 33.6 | 11.2 | CR10198 | 2.2 | 0.9 | CR10293 | 16.4 | 5.6 |
| CR10108 | 61.6 | 13.7 | CR10199 | 14.0 | 3.7 | CR10294 | 35.1 | 7.9 |
| CR10109 | 50.5 | 12.9 | CR10200 | 4.5 | 2.4 | CR10295 | 34.1 | 2.8 |
| CR10110 | 40.7 | 11.5 | CR10201 | 1.0 | 0.5 | CR10296 | 11.3 | 3.5 |
| CR10111 | 59.6 | 18.7 | CR10202 | 5.2 | 0.9 | CR10297 | 22.6 | 1.2 |
| CR10112 | 13.8 | 11.5 | CR10203 | 12.4 | 2.2 | CR10298 | 58.5 | 5.2 |
| CR10113 | 82.2 | 7.0 | CR10204 | 28.6 | 8.2 | CR10299 | 70.8 | 10.4 |
| CR10114 | 36.2 | 5.7 | CR10205 | 3.4 | 1.0 | CR10300 | 50.2 | 14.2 |
| CR10115 | 77.2 | 7.1 | CR10206 | 13.6 | 1.4 | CR10301 | 63.4 | 10.9 |
| CR10116 | 38.6 | 4.4 | CR10207 | 1.6 | 0.3 | CR10302 | 24.5 | 12.4 |
| CR10117 | 73.1 | 6.8 | CR10208 | 12.3 | 2.5 | CR10303 | 49.2 | 11.6 |
| CR10118 | 76.0 | 3.3 | CR10209 | 15.9 | 2.9 | CR10304 | 58.7 | 13.6 |
| CR10119 | 78.5 | 3.6 | CR10210 | 9.7 | 1.5 | CR10305 | 43.5 | 10.0 |
| CR10120 | 72.6 | 6.7 | CR10211 | 6.2 | 1.3 | CR10306 | 31.5 | 10.0 |
| CR10121 | 71.6 | 9.8 | CR10212 | 10.7 | 0.6 | CR10307 | 57.9 | 16.1 |
| CR10122 | 77.2 | 5.8 | CR10213 | 21.1 | 6.1 | CR10308 | 55.7 | 5.6 |
| CR10123 | 50.3 | 11.7 | CR10214 | 5.7 | 2.2 | CR10309 | 74.5 | 4.3 |
| CR09734 | 66.6 | 7.6 | CR10215 | 9.5 | 1.7 | CR10310 | 56.6 | 4.1 |
| CR09723 | 62.5 | 14.2 | CR10216 | 6.0 | 1.2 | CR10311 | 55.1 | 6.0 |
| CR09726 | 46.1 | 17.4 | CR10217 | 2.8 | 0.9 | CR10312 | 13.3 | 3.2 |
| CR10124 | 23.5 | 12.2 | CR10218 | 1.2 | 0.1 | CR10313 | 21.8 | 2.2 |
| CR10125 | 49.8 | 9.5 | CR10219 | 5.0 | 0.8 | CR10314 | 32.0 | 5.6 |
| CR10126 | 46.8 | 8.4 | CR10220 | 4.2 | 0.9 | CR10315 | 18.6 | 0.6 |
| CR10127 | 42.8 | 13.7 | CR10221 | 4.1 | 0.5 | CR10316 | 11.3 | 0.9 |
| CR10128 | 37.2 | 19.3 | CR10222 | 2.1 | 0.6 | CR10317 | 12.1 | 3.9 |
| CR10129 | 49.9 | 13.5 | CR10223 | 7.3 | 0.7 | CR10318 | 26.4 | 5.2 |
| CR10600 | 64.9 | 4.7 | CR10691 | 15.2 | 2.4 | CR10785 | 75.2 | 3.6 |
| CR10601 | 33.1 | 4.2 | CR10692 | 1.4 | 0.3 | CR10786 | 48.5 | 7.7 |
| CR10602 | 2.0 | 0.2 | CR10693 | 0.9 | 0.2 | CR10787 | 29.1 | 3.6 |
| CR10603 | 45.4 | 2.9 | CR10694 | 10.6 | 0.6 | CR10788 | 65.6 | 6.9 |
| CR10604 | 49.9 | 3.4 | CR10695 | 9.7 | 0.8 | CR10789 | 62.7 | 1.3 |
| CR10605 | 42.0 | 3.4 | CR10696 | 4.2 | 0.4 | CR10790 | 58.9 | 4.2 |
| CR10606 | 57.7 | 4.7 | CR10697 | 6.5 | 0.4 | CR10791 | 71.4 | 3.6 |
| CR10607 | 66.0 | 2.2 | CR10698 | 8.8 | 1.8 | CR10792 | 64.4 | 8.2 |
| CR10608 | 72.2 | 5.1 | CR10699 | 15.0 | 2.9 | CR10793 | 61.8 | 2.2 |
| CR10609 | 50.7 | 4.1 | CR10700 | 12.2 | 0.7 | CR10794 | 71.9 | 5.3 |
| CR10610 | 75.2 | 3.6 | CR10701 | 15.0 | 2.9 | CR10795 | 75.3 | 6.8 |
| CR10611 | 2.6 | 0.5 | CR10702 | 0.3 | 0.1 | CR10796 | 2.9 | 1.1 |
| CR10612 | 25.8 | 8.3 | CR10703 | 12.2 | 1.9 | CR10797 | 44.7 | 7.7 |
| CR10613 | 30.8 | 9.2 | CR10704 | 8.7 | 1.3 | CR10798 | 30.8 | 5.1 |
| CR10614 | 28.9 | 4.8 | CR10705 | 5.1 | 0.5 | CR10799 | 28.0 | 4.1 |
| CR10615 | 25.0 | 4.3 | CR10706 | 5.7 | 0.5 | CR10800 | 22.2 | 2.2 |
| CR10616 | 28.5 | 7.6 | CR10707 | 8.7 | 2.5 | CR10801 | 27.8 | 4.9 |
| CR10617 | 24.7 | 3.1 | CR10708 | 8.6 | 0.8 | CR10802 | 26.9 | 5.0 |
| CR10618 | 0.6 | 0.1 | CR10709 | 0.8 | 0.2 | CR10803 | 6.2 | 0.5 |
| CR10619 | 39.1 | 4.5 | CR10710 | 13.3 | 1.9 | CR10804 | 27.8 | 4.2 |
| CR10620 | 56.3 | 4.2 | CR10711 | 15.1 | 3.9 | CR10805 | 55.1 | 9.8 |
| CR10621 | 3.4 | 0.4 | CR10712 | 0.4 | 0.1 | CR10806 | 1.7 | 0.4 |
| CR10622 | 8.4 | 1.4 | CR10713 | 1.2 | 0.2 | CR10807 | 10.8 | 2.3 |
| CR10623 | 19.9 | 3.8 | CR10714 | 3.2 | 0.8 | CR10808 | 12.0 | 1.9 |
| CR10624 | 22.4 | 1.6 | CR10715 | 9.2 | 2.6 | CR10809 | 20.3 | 5.2 |
| CR10625 | 24.9 | 1.1 | CR10716 | 8.8 | 1.1 | CR10810 | 26.6 | 4.1 |
| CR10626 | 11.5 | 1.1 | CR10717 | 1.1 | 0.3 | CR10811 | 11.9 | 2.6 |
| CR10627 | 19.1 | 1.5 | CR10718 | 7.5 | 2.3 | CR10812 | 34.8 | 4.4 |
| CR10628 | 25.9 | 4.8 | CR10719 | 7.1 | 1.3 | CR10813 | 15.6 | 3.2 |
| CR10629 | 23.2 | 3.3 | CR10720 | 9.3 | 2.1 | CR10814 | 17.7 | 2.4 |
| CR10630 | 34.8 | 3.2 | CR10721 | 10.2 | 5.3 | CR10815 | 29.3 | 6.6 |
| CR10631 | 6.1 | 4.9 | CR10722 | 0.2 | 0.1 | CR10816 | 1.4 | 0.2 |
| CR10632 | 3.0 | 0.4 | CR10723 | 1.2 | 0.2 | CR10817 | 18.5 | 3.5 |
| CR10633 | 25.3 | 4.4 | CR10724 | 9.5 | 1.9 | CR10818 | 33.6 | 5.9 |
| CR10634 | 1.0 | 0.0 | CR10725 | 0.3 | 0.1 | CR10819 | 1.3 | 0.1 |
| CR10635 | 34.4 | 2.7 | CR10726 | 11.5 | 3.3 | CR10820 | 17.0 | 2.3 |
| CR10636 | 11.6 | 1.4 | CR10727 | 1.0 | 0.2 | CR10821 | 8.8 | 1.1 |
| CR10637 | 0.4 | 0.1 | CR10728 | 0.9 | 0.1 | CR10822 | 4.8 | 0.4 |
| CR10638 | 21.9 | 4.6 | CR10729 | 7.4 | 3.2 | CR10823 | 27.5 | 5.1 |
| CR10639 | 22.1 | 5.6 | CR10730 | 3.1 | 0.9 | CR10824 | 12.9 | 1.1 |
| CR10640 | 30.0 | 2.0 | CR10731 | 7.9 | 2.0 | CR10825 | 29.4 | 6.7 |
| CR10641 | 54.1 | 1.9 | CR10732 | 14.0 | 5.5 | CR10826 | 38.4 | 2.5 |
| CR10642 | 34.3 | 3.0 | CR10733 | 1.7 | 0.3 | CR10827 | 41.0 | 3.8 |
| CR10643 | 1.3 | 0.4 | CR10734 | 0.8 | 0.2 | CR10828 | 8.7 | 2.4 |
| CR10644 | 19.5 | 3.9 | CR10735 | 8.2 | 1.7 | CR10829 | 16.7 | 3.0 |
| CR10645 | 22.8 | 1.5 | CR10736 | 16.5 | 6.2 | CR10830 | 18.9 | 6.4 |
| CR10646 | 29.2 | 3.2 | CR10737 | 9.9 | 0.9 | CR10831 | 30.6 | 1.7 |
| CR10647 | 1.0 | 0.1 | CR10738 | 0.3 | 0.1 | CR10832 | 1.2 | 0.3 |
| CR10648 | 20.6 | 6.6 | CR10739 | 6.7 | 0.8 | CR10833 | 21.2 | 5.7 |
| CR10649 | 19.3 | 2.5 | CR10740 | 4.9 | 0.8 | CR10834 | 28.2 | 4.6 |
| CR10650 | 22.2 | 3.1 | CR10741 | 6.2 | 0.7 | CR10835 | 20.2 | 0.3 |
| CR10651 | 29.8 | 5.7 | CR10742 | 6.0 | 1.7 | CR10836 | 12.0 | 2.4 |
| CR10652 | 1.9 | 0.2 | CR10743 | 0.3 | 0.1 | CR10837 | 1.7 | 0.2 |
| CR10653 | 2.3 | 0.4 | CR10744 | 0.3 | 0.1 | CR10838 | 3.0 | 1.1 |
| CR10654 | 25.8 | 3.6 | CR10745 | 11.8 | 5.7 | CR10839 | 17.5 | 3.1 |
| CR10655 | 23.8 | 3.4 | CR10746 | 8.5 | 2.0 | CR10840 | 17.0 | 0.6 |
| CR10656 | 0.8 | 0.4 | CR10747 | 0.5 | 0.2 | CR10841 | 4.6 | 1.0 |
| CR10657 | 18.0 | 4.6 | CR10748 | 1.4 | 0.4 | CR10842 | 10.8 | 3.1 |
| CR10658 | 21.8 | 1.9 | CR10749 | 8.5 | 1.3 | CR10843 | 14.6 | 1.9 |
| CR10659 | 1.0 | 0.1 | CR10750 | 0.2 | 0.1 | CR10844 | 0.4 | 0.0 |
| CR10660 | 26.8 | 1.6 | CR10751 | 8.4 | 2.9 | CR10845 | 19.5 | 2.9 |
| CR10661 | 20.9 | 0.6 | CR10752 | 6.7 | 2.3 | CR10846 | 19.2 | 2.4 |
| CR10662 | 34.2 | 1.4 | CR10753 | 5.6 | 3.8 | CR10847 | 15.9 | 1.0 |
| CR10663 | 54.8 | 2.7 | CR10754 | 35.2 | 23.3 | CR10848 | 38.7 | 2.6 |
| CR10664 | 46.8 | 4.0 | CR10755 | 9.5 | 3.8 | CR10849 | 28.8 | 4.0 |
| CR10665 | 0.3 | 0.2 | CR10756 | 0.2 | 0.1 | CR10850 | 0.3 | 0.1 |
| CR10666 | 0.9 | 0.2 | CR10757 | 0.5 | 0.1 | CR10851 | 4.7 | 1.6 |
| CR10667 | 40.8 | 6.0 | CR10758 | 9.4 | 1.7 | CR10852 | 34.4 | 8.8 |
| CR10668 | 0.7 | 0.3 | CR10759 | 0.5 | 0.1 | CR10853 | 4.8 | 1.6 |
| CR10669 | 32.0 | 9.7 | CR10760 | 1.9 | 0.6 | CR10854 | 23.6 | 6.3 |
| CR10670 | 37.8 | 13.5 | CR10761 | 10.2 | 1.9 | CR10855 | 21.5 | 7.3 |
| CR10671 | 1.3 | 0.4 | CR10762 | 0.2 | 0.0 | CR10856 | 0.6 | 0.2 |
| CR10672 | 38.2 | 2.8 | CR10763 | 7.8 | 2.6 | CR10857 | 20.8 | 2.6 |
| CR10673 | 7.2 | 1.1 | CR10764 | 0.3 | 0.1 | CR10858 | 1.4 | 0.5 |
| CR10674 | 81.0 | 2.3 | CR10765 | 29.6 | 7.0 | CR10859 | 59.8 | 8.9 |
| CR10675 | 2.1 | 0.3 | CR10766 | 1.2 | 0.5 | CR10860 | 6.9 | 3.3 |
| CR10676 | 41.3 | 7.7 | CR10767 | 2.0 | 0.4 | CR10861 | 22.1 | 10.2 |
| CR10677 | 0.2 | 0.1 | CR10768 | 0.2 | 0.1 | CR10862 | 0.4 | 0.2 |
| CR10678 | 26.2 | 2.7 | CR10769 | 2.7 | 0.7 | CR10863 | 31.4 | 10.7 |
| CR10679 | 71.1 | 3.1 | CR10770 | 26.4 | 8.9 | CR10864 | 61.6 | 13.5 |
| CR10680 | 57.8 | 7.9 | CR10771 | 8.7 | 4.7 | CR10865 | 60.2 | 10.0 |
| CR10681 | 3.6 | 0.6 | CR10772 | 0.3 | 0.1 | CR10866 | 1.4 | 0.4 |
| CR10682 | 69.4 | 1.6 | CR10773 | 32.8 | 12.4 | CR10867 | 47.8 | 6.2 |
| CR10683 | 33.4 | 10.7 | CR10774 | 1.2 | 0.5 | CR10868 | 29.6 | 6.6 |
| CR10684 | 5.1 | 1.8 | CR10775 | 2.0 | 1.4 | CR10869 | 20.1 | 7.3 |
| CR09687 | 38.3 | 3.2 | CR10776 | 3.2 | 0.6 | CR10870 | 37.3 | 2.2 |
| CR09676 | 67.8 | 6.9 | CR10777 | 17.2 | 7.9 | CR10871 | 56.7 | 4.5 |
| CR09679 | 25.0 | 4.3 | CR10778 | 7.6 | 2.6 | CR10872 | 18.1 | 2.3 |
| CR10685 | 0.4 | 0.3 | CR10779 | 0.5 | 0.1 | CR10873 | 6.9 | 1.0 |
| CR10686 | 24.3 | 3.8 | CR10780 | 6.4 | 0.8 | CR10874 | 24.1 | 5.0 |
| CR10687 | 34.6 | 3.5 | CR10781 | 10.6 | 3.4 | CR10875 | 20.0 | 3.7 |
| CR10688 | 27.1 | 3.4 | CR10782 | 4.0 | 1.1 | CR10876 | 7.3 | 0.5 |
| CR10689 | 0.3 | 0.0 | CR10783 | 0.2 | 0.1 | CR10877 | 0.5 | 0.2 |
| CR10690 | 11.9 | 2.4 | CR10784 | 0.4 | 0.1 | CR10878 | 6.3 | 1.4 |
| CR11158 | 79.4 | 4.1 | CR11252 | 11.9 | 1.3 | CR11346 | 89.0 | 2.5 |
| CR11159 | 74.6 | 1.2 | CR11253 | 18.6 | 1.7 | CR11347 | 77.9 | 5.0 |
| CR11160 | 63.3 | 4.1 | CR11254 | 9.2 | 0.5 | CR11348 | 69.2 | 3.3 |
| CR11161 | 58.6 | 2.6 | CR11255 | 4.9 | 0.6 | CR11349 | 69.9 | 6.0 |
| CR11162 | 67.8 | 2.3 | CR11256 | 18.5 | 0.9 | CR11350 | 73.4 | 2.6 |
| CR11163 | 53.4 | 7.1 | CR11257 | 2.5 | 0.6 | CR11351 | 72.1 | 5.5 |
| CR11164 | 63.6 | 2.2 | CR11258 | 12.4 | 2.8 | CR11352 | 70.4 | 1.7 |
| CR11165 | 65.2 | 2.2 | CR11259 | 5.2 | 0.9 | CR11353 | 66.9 | 2.7 |
| CR11166 | 52.6 | 5.2 | CR11260 | 5.9 | 1.0 | CR11354 | 61.6 | 3.5 |
| CR11167 | 68.3 | 3.6 | CR11261 | 18.9 | 1.6 | CR11355 | 67.1 | 3.7 |
| CR11168 | 67.8 | 1.5 | CR11262 | 4.0 | 0.6 | CR11356 | 82.6 | 1.5 |
| CR11169 | 59.5 | 9.5 | CR11263 | 8.4 | 4.9 | CR11357 | 68.2 | 4.8 |
| CR11170 | 60.1 | 16.5 | CR11264 | 10.4 | 4.2 | CR11358 | 49.5 | 0.9 |
| CR11171 | 61.5 | 18.2 | CR11265 | 7.3 | 1.6 | CR11359 | 43.4 | 4.8 |
| CR11172 | 30.0 | 11.7 | CR11266 | 1.6 | 0.1 | CR11360 | 37.7 | 5.0 |
| CR11173 | 44.7 | 19.6 | CR11267 | 1.6 | 0.3 | CR11361 | 33.9 | 1.3 |
| CR11174 | 56.0 | 15.1 | CR11268 | 8.5 | 0.8 | CR11362 | 37.5 | 6.1 |
| CR11175 | 35.8 | 1.6 | CR11269 | 0.8 | 0.2 | CR11363 | 41.0 | 7.2 |
| CR11176 | 32.0 | 8.9 | CR11270 | 1.3 | 0.2 | CR11364 | 30.7 | 1.8 |
| CR11177 | 61.8 | 10.4 | CR11271 | 9.3 | 2.3 | CR11365 | 49.5 | 3.7 |
| CR11178 | 71.5 | 6.7 | CR11272 | 12.1 | 2.1 | CR11366 | 65.8 | 1.1 |
| CR11179 | 73.9 | 16.4 | CR11273 | 34.8 | 33.7 | CR11367 | 66.8 | 2.9 |
| CR11180 | 64.3 | 12.3 | CR11274 | 9.0 | 7.6 | CR11368 | 46.5 | 7.1 |
| CR11181 | 48.0 | 6.8 | CR11275 | 4.2 | 0.3 | CR11369 | 35.9 | 6.1 |
| CR11182 | 35.3 | 5.6 | CR11276 | 3.2 | 0.2 | CR11370 | 32.5 | 11.9 |
| CR11183 | 41.4 | 17.1 | CR11277 | 2.7 | 1.1 | CR11371 | 31.1 | 10.2 |
| CR11184 | 51.4 | 17.7 | CR11278 | 3.6 | 0.8 | CR11372 | 22.8 | 4.1 |
| CR11185 | 44.4 | 8.5 | CR11279 | 5.6 | 2.0 | CR11373 | 29.5 | 8.8 |
| CR11186 | 46.5 | 11.0 | CR11280 | 3.9 | 0.9 | CR11374 | 27.1 | 8.0 |
| CR11187 | 24.8 | 13.7 | CR11281 | 0.6 | 0.1 | CR11375 | 27.2 | 2.7 |
| CR11188 | 66.4 | 5.4 | CR11282 | 9.3 | 2.7 | CR11376 | 30.6 | 2.6 |
| CR11189 | 55.0 | 3.7 | CR11283 | 4.3 | 0.6 | CR11377 | 49.5 | 7.9 |
| CR11190 | 61.5 | 18.4 | CR11284 | 6.4 | 1.5 | CR11378 | 51.7 | 6.9 |
| CR11191 | 55.0 | 11.2 | CR11285 | 25.5 | 16.2 | CR11379 | 24.3 | 8.1 |
| CR11192 | 38.0 | 11.0 | CR11286 | 2.2 | 1.1 | CR11380 | 15.1 | 2.8 |
| CR11193 | 53.4 | 8.0 | CR11287 | 10.3 | 4.8 | CR11381 | 33.0 | 12.9 |
| CR11194 | 20.6 | 1.2 | CR11288 | 1.0 | 0.2 | CR11382 | 15.6 | 5.5 |
| CR11195 | 39.2 | 7.3 | CR11289 | 1.8 | 0.8 | CR11383 | 17.9 | 5.4 |
| CR11196 | 59.4 | 15.0 | CR11290 | 6.2 | 0.6 | CR11384 | 29.1 | 11.8 |
| CR11197 | 29.4 | 5.1 | CR11291 | 1.4 | 0.1 | CR11385 | 25.7 | 8.8 |
| CR11198 | 35.2 | 8.1 | CR11292 | 1.5 | 0.4 | CR11386 | 26.5 | 2.8 |
| CR11199 | 72.5 | 6.7 | CR11293 | 11.5 | 2.6 | CR11387 | 49.8 | 10.4 |
| CR11200 | 64.5 | 9.7 | CR11294 | 15.4 | 2.5 | CR11388 | 55.4 | 2.0 |
| CR11201 | 45.5 | 38.7 | CR11295 | 3.3 | 0.2 | CR11389 | 30.1 | 10.1 |
| CR11202 | 37.8 | 20.4 | CR11296 | 2.6 | 1.2 | CR11390 | 18.3 | 2.7 |
| CR11203 | 19.3 | 4.5 | CR11297 | 1.6 | 0.5 | CR11391 | 20.6 | 3.8 |
| CR11204 | 61.3 | 22.2 | CR11298 | 4.5 | 0.7 | CR11392 | 17.7 | 3.4 |
| CR11205 | 58.5 | 20.1 | CR11299 | 10.5 | 2.7 | CR11393 | 32.5 | 3.7 |
| CR11206 | 40.2 | 4.2 | CR11300 | 9.2 | 5.1 | CR11394 | 21.5 | 2.7 |
| CR11207 | 28.5 | 27.5 | CR11301 | 0.8 | 0.3 | CR11395 | 8.5 | 1.7 |
| CR11208 | 39.9 | 9.6 | CR11302 | 6.7 | 2.6 | CR11396 | 22.4 | 3.0 |
| CR11209 | 43.8 | 8.4 | CR11303 | 3.1 | 0.4 | CR11397 | 19.8 | 3.1 |
| CR11210 | 58.5 | 8.6 | CR11304 | 9.4 | 3.0 | CR11398 | 37.2 | 5.2 |
| CR11211 | 61.1 | 8.1 | CR11305 | 3.3 | 1.7 | CR11399 | 44.5 | 6.1 |
| CR11212 | 16.5 | 2.4 | CR11306 | 6.9 | 9.2 | CR11400 | 20.3 | 3.4 |
| CR11213 | 35.8 | 6.5 | CR11307 | 7.3 | 2.6 | CR11401 | 16.7 | 5.2 |
| CR11214 | 42.2 | 11.5 | CR11308 | 5.8 | 2.7 | CR11402 | 25.7 | 5.4 |
| CR11215 | 29.7 | 9.3 | CR11309 | 2.3 | 0.8 | CR11403 | 11.7 | 1.2 |
| CR11216 | 25.9 | 5.1 | CR11310 | 7.2 | 4.5 | CR11404 | 17.4 | 5.7 |
| CR11217 | 16.0 | 4.6 | CR11311 | 0.9 | 0.4 | CR11405 | 13.4 | 3.7 |
| CR11218 | 32.2 | 15.3 | CR11312 | 2.4 | 1.0 | CR11406 | 10.8 | 4.1 |
| CR11219 | 25.4 | 11.6 | CR11313 | 3.6 | 2.2 | CR11407 | 13.4 | 6.3 |
| CR11220 | 41.7 | 17.9 | CR11314 | 6.7 | 2.0 | CR11408 | 23.4 | 3.9 |
| CR11221 | 30.3 | 10.1 | CR11315 | 3.0 | 1.0 | CR11409 | 22.3 | 5.0 |
| CR11222 | 45.3 | 26.9 | CR11316 | 2.9 | 0.9 | CR11410 | 22.9 | 9.8 |
| CR11223 | 17.7 | 2.8 | CR11317 | 1.2 | 0.3 | CR11411 | 15.1 | 4.9 |
| CR11224 | 29.2 | 1.9 | CR11318 | 1.5 | 0.2 | CR11412 | 12.7 | 3.5 |
| CR11225 | 17.1 | 1.1 | CR11319 | 1.5 | 0.1 | CR11413 | 8.0 | 2.3 |
| CR11226 | 25.2 | 4.1 | CR11320 | 1.5 | 0.6 | CR11414 | 6.6 | 1.9 |
| CR11227 | 22.3 | 2.0 | CR11321 | 1.7 | 0.0 | CR11415 | 10.2 | 4.3 |
| CR11228 | 21.8 | 3.4 | CR11322 | 1.5 | 0.8 | CR11416 | 7.5 | 3.5 |
| CR11229 | 11.4 | 0.8 | CR11323 | 1.1 | 0.3 | CR11417 | 3.8 | 1.2 |
| CR11230 | 28.5 | 5.8 | CR11324 | 0.2 | 0.1 | CR11418 | 9.5 | 2.9 |
| CR11231 | 52.6 | 1.7 | CR11325 | 5.0 | 2.5 | CR11419 | 16.9 | 2.0 |
| CR11232 | 58.5 | 5.3 | CR11326 | 13.3 | 5.5 | CR11420 | 34.3 | 8.3 |
| CR11233 | 51.3 | 8.3 | CR11327 | 11.0 | 3.6 | CR11421 | 22.5 | 6.4 |
| CR11234 | 48.1 | 4.1 | CR11328 | 5.4 | 2.0 | CR11422 | 20.6 | 6.0 |
| CR11235 | 40.5 | 19.6 | CR11329 | 3.6 | 3.3 | CR11423 | 20.8 | 6.8 |
| CR11236 | 47.1 | 13.6 | CR11330 | 2.7 | 1.4 | CR11424 | 17.8 | 4.9 |
| CR11237 | 47.0 | 8.3 | CR11331 | 4.9 | 1.8 | CR11425 | 22.9 | 7.1 |
| CR11238 | 39.0 | 15.6 | CR11332 | 3.6 | 2.4 | CR11426 | 13.9 | 4.1 |
| CR11239 | 56.7 | 11.8 | CR11333 | 5.4 | 1.4 | CR11427 | 14.7 | 3.3 |
| CR11240 | 52.2 | 18.7 | CR11334 | 10.8 | 8.6 | CR11428 | 13.5 | 2.9 |
| CR11241 | 64.9 | 15.8 | CR11335 | 10.8 | 8.6 | CR11429 | 17.1 | 3.4 |
| CR11242 | 74.6 | 19.2 | CR11336 | 10.8 | 8.6 | CR11430 | 16.1 | 4.4 |
| CR11243 | 33.7 | 1.5 | CR11337 | 0.9 | 0.1 | CR11431 | 19.2 | 1.2 |
| CR11244 | 48.2 | 6.2 | CR11338 | 30.1 | 25.1 | CR11432 | 58.3 | 7.1 |
| CR11245 | 32.6 | 8.0 | CR11339 | 2.8 | 1.2 | CR11433 | 22.8 | 8.7 |
| CR11246 | 54.2 | 11.1 | CR11340 | 4.0 | 1.1 | CR11434 | 37.1 | 5.5 |
| CR11247 | 39.4 | 11.5 | CR11341 | 4.4 | 2.0 | CR11435 | 24.2 | 2.4 |
| CR11248 | 41.0 | 1.9 | CR11342 | 7.1 | 0.3 | CR11436 | 24.0 | 5.8 |
| CR11249 | 69.4 | 21.0 | CR11343 | 3.7 | 1.3 | CR11437 | 15.9 | 5.7 |
| CR11250 | 17.9 | 9.4 | CR11344 | 1.0 | 0.4 | CR11438 | 11.7 | 3.1 |
| CR11251 | 20.7 | 4.7 | CR11345 | 1.6 | 0.5 | CR11439 | 14.9 | 4.2 |

| G494 Var | Edit | SD | G499 Var | Edit | SD | G502 Var | Edit | SD |
|---|---|---|---|---|---|---|---|---|
| CR10414 | 18.3 | 3.8 | CR10509 | 19.8 | 6.6 | CR09618 | 8.4 | 3.7 |
| CR03360 | 38.2 | 8.2 | CR05303 | 16.6 | 5.6 | CR00689 | 9.8 | 4.8 |
| CR10320 | 51.7 | 5.5 | CR10415 | 39.6 | 1.3 | CR10510 | 24.3 | 3.4 |
| CR10321 | 56.1 | 7.1 | CR10416 | 49.9 | 6.6 | CR10511 | 22.8 | 0.5 |
| CR10322 | 46.3 | 3.8 | CR10417 | 25.9 | 4.3 | CR10512 | 8.3 | 4.1 |
| CR10323 | 48.1 | 1.2 | CR10418 | 45.9 | 7.8 | CR10513 | 10.5 | 1.1 |
| CR10324 | 48.8 | 2.6 | CR10419 | 35.3 | 2.8 | CR10514 | 10.6 | 0.8 |
| CR10325 | 53.1 | 2.7 | CR10420 | 43.4 | 5.0 | CR10515 | 17.7 | 7.9 |
| CR10326 | 48.5 | 4.4 | CR10421 | 46.0 | 2.4 | CR10516 | 19.0 | 5.7 |
| CR10327 | 56.9 | 3.1 | CR10422 | 38.0 | 3.0 | CR10517 | 24.6 | 4.7 |
| CR10328 | 52.2 | 5.7 | CR10423 | 39.4 | 4.2 | CR10518 | 17.6 | 0.9 |
| CR10329 | 55.7 | 5.5 | CR10424 | 41.7 | 8.5 | CR10519 | 19.7 | 3.8 |
| CR10330 | 54.5 | 6.4 | CR10425 | 45.4 | 7.6 | CR10520 | 17.3 | 15.0 |
| CR10331 | 42.1 | 8.6 | CR10426 | 26.7 | 4.3 | CR10521 | 13.7 | 3.7 |
| CR10332 | 43.6 | 12.6 | CR10427 | 24.3 | 5.7 | CR10522 | 21.8 | 4.8 |
| CR10333 | 36.2 | 3.8 | CR10428 | 32.3 | 10.9 | CR10523 | 8.7 | 1.6 |
| CR10334 | 28.7 | 5.9 | CR10429 | 31.3 | 2.0 | CR10524 | 7.4 | 0.9 |
| CR10335 | 31.7 | 13.3 | CR10430 | 31.9 | 5.4 | CR10525 | 8.1 | 0.8 |
| CR10336 | 20.7 | 7.5 | CR10431 | 33.4 | 5.9 | CR10526 | 6.3 | 1.0 |
| CR10337 | 28.0 | 11.1 | CR10432 | 30.4 | 6.3 | CR10527 | 5.5 | 0.5 |
| CR10338 | 12.3 | 4.0 | CR10433 | 20.2 | 6.2 | CR10528 | 3.1 | 0.1 |
| CR10339 | 22.5 | 3.2 | CR10434 | 28.1 | 6.1 | CR10529 | 7.8 | 1.4 |
| CR10340 | 45.2 | 1.8 | CR10435 | 46.9 | 6.8 | CR10530 | 28.3 | 6.2 |
| CR10341 | 41.2 | 10.3 | CR10436 | 24.7 | 3.3 | CR10531 | 12.8 | 2.8 |
| CR10342 | 18.5 | 8.5 | CR10437 | 17.7 | 3.0 | CR10532 | 8.8 | 2.3 |
| CR10343 | 16.9 | 3.6 | CR10438 | 20.7 | 1.6 | CR10533 | 11.8 | 1.1 |
| CR10344 | 16.2 | 2.9 | CR10439 | 24.1 | 1.6 | CR10534 | 5.4 | 1.2 |
| CR10345 | 18.8 | 5.3 | CR10440 | 28.4 | 4.3 | CR10535 | 7.7 | 2.2 |
| CR10346 | 10.8 | 3.2 | CR10441 | 26.0 | 3.2 | CR10536 | 6.5 | 0.8 |
| CR10347 | 19.7 | 5.8 | CR10442 | 31.8 | 5.5 | CR10537 | 10.7 | 0.5 |
| CR10348 | 19.2 | 1.5 | CR10443 | 29.7 | 0.7 | CR09660 | 6.8 | 1.7 |
| CR10349 | 12.3 | 2.5 | CR10444 | 17.9 | 2.4 | CR10538 | 4.4 | 0.7 |
| CR10350 | 24.3 | 3.8 | CR10445 | 27.2 | 4.0 | CR10539 | 6.5 | 0.8 |
| CR10351 | 35.0 | 0.5 | CR10446 | 23.1 | 2.7 | CR10540 | 4.9 | 0.6 |
| CR10352 | 35.5 | 8.6 | CR10447 | 25.9 | 2.4 | CR10541 | 9.8 | 3.7 |
| CR10353 | 31.4 | 10.1 | CR10448 | 24.0 | 3.5 | CR10542 | 12.3 | 4.0 |
| CR10354 | 15.2 | 7.0 | CR10449 | 17.7 | 4.0 | CR10543 | 3.2 | 0.4 |
| CR10355 | 27.1 | 5.2 | CR10450 | 32.3 | 1.1 | CR10544 | 7.6 | 1.4 |
| CR10356 | 27.8 | 6.5 | CR10451 | 30.7 | 6.6 | CR10545 | 8.8 | 1.0 |
| CR10357 | 8.0 | 2.5 | CR10452 | 14.1 | 2.9 | CR10546 | 2.1 | 0.2 |
| CR10358 | 15.3 | 2.1 | CR10453 | 25.4 | 1.7 | CR10547 | 8.0 | 1.7 |
| CR10359 | 16.7 | 3.9 | CR10454 | 25.2 | 4.2 | CR10548 | 10.8 | 1.0 |
| CR10360 | 28.1 | 4.6 | CR10455 | 29.7 | 6.3 | CR10549 | 9.2 | 2.5 |
| CR10361 | 35.7 | 1.6 | CR10456 | 42.0 | 6.8 | CR10550 | 11.8 | 0.8 |
| CR10362 | 44.2 | 5.6 | CR10457 | 39.8 | 4.4 | CR10551 | 21.9 | 12.2 |
| CR10363 | 22.7 | 7.7 | CR10458 | 20.0 | 6.7 | CR10552 | 5.1 | 2.2 |
| CR10364 | 36.6 | 7.1 | CR10459 | 25.7 | 7.4 | CR10553 | 7.7 | 4.6 |
| CR10365 | 19.4 | 4.3 | CR10460 | 32.3 | 4.8 | CR10554 | 5.8 | 0.8 |
| CR10366 | 16.6 | 1.0 | CR10461 | 31.5 | 3.3 | CR10555 | 11.0 | 4.9 |
| CR10367 | 14.1 | 3.5 | CR10462 | 16.6 | 3.6 | CR10556 | 2.4 | 0.5 |
| CR10368 | 15.6 | 7.0 | CR10463 | 26.4 | 4.6 | CR10557 | 4.5 | 0.3 |
| CR10369 | 15.7 | 0.8 | CR10464 | 23.5 | 4.4 | CR10558 | 9.4 | 0.9 |
| CR10370 | 14.9 | 3.6 | CR10465 | 28.1 | 4.0 | CR10559 | 4.8 | 1.5 |
| CR10371 | 28.5 | 4.4 | CR10466 | 25.2 | 3.6 | CR10560 | 4.9 | 1.0 |
| CR10372 | 28.0 | 0.3 | CR10467 | 32.1 | 2.7 | CR10561 | 4.6 | 1.4 |
| CR10373 | 38.3 | 5.1 | CR10468 | 34.8 | 8.7 | CR10562 | 16.7 | 7.1 |
| CR10374 | 18.0 | 3.9 | CR10469 | 28.6 | 5.5 | CR10563 | 7.2 | 1.7 |
| CR10375 | 15.2 | 1.8 | CR10470 | 33.5 | 8.9 | CR10564 | 7.8 | 1.7 |
| CR10376 | 12.6 | 1.2 | CR10471 | 27.3 | 7.2 | CR10565 | 4.6 | 1.6 |
| CR10377 | 17.9 | 3.5 | CR10472 | 28.9 | 4.5 | CR10566 | 8.1 | 3.5 |
| CR10378 | 14.8 | 4.9 | CR10473 | 33.8 | 8.7 | CR10567 | 9.9 | 4.0 |
| CR10379 | 12.9 | 0.9 | CR10474 | 24.5 | 4.5 | CR10568 | 4.5 | 1.4 |
| CR10380 | 14.5 | 3.2 | CR10475 | 25.7 | 5.0 | CR10569 | 9.2 | 3.0 |
| CR10381 | 20.0 | 1.3 | CR10476 | 31.5 | 5.2 | CR10570 | 6.8 | 2.3 |
| CR10382 | 19.3 | 2.4 | CR10477 | 31.3 | 8.7 | CR10571 | 8.8 | 2.8 |
| CR10383 | 42.1 | 5.9 | CR10478 | 42.0 | 5.0 | CR10572 | 11.1 | 2.2 |
| CR10384 | 36.4 | 1.5 | CR10479 | 26.9 | 5.5 | CR10573 | 11.1 | 3.8 |
| CR10385 | 18.2 | 1.1 | CR10480 | 18.4 | 2.6 | CR10574 | 3.8 | 0.7 |
| CR10386 | 12.8 | 1.9 | CR10481 | 25.1 | 3.7 | CR10575 | 11.3 | 11.1 |
| CR10387 | 16.4 | 1.0 | CR10482 | 27.1 | 3.3 | CR10576 | 12.6 | 2.1 |
| CR10388 | 14.5 | 3.7 | CR10483 | 19.2 | 2.9 | CR10577 | 4.2 | 1.2 |
| CR10389 | 16.6 | 3.4 | CR10484 | 25.8 | 5.9 | CR10578 | 7.0 | 2.1 |
| CR10390 | 18.3 | 1.9 | CR10485 | 21.6 | 2.3 | CR10579 | 5.6 | 1.8 |
| CR10391 | 11.3 | 3.1 | CR10486 | 16.3 | 2.7 | CR10580 | 2.1 | 0.9 |
| CR10392 | 21.3 | 7.3 | CR10487 | 24.2 | 4.0 | CR10581 | 11.5 | 2.5 |
| CR10393 | 43.1 | 3.4 | CR10488 | 34.7 | 2.5 | CR10582 | 11.7 | 5.3 |
| CR10394 | 53.7 | 6.0 | CR10489 | 57.2 | 6.9 | CR10583 | 18.8 | 13.9 |
| CR10395 | 45.8 | 2.5 | CR10490 | 37.2 | 6.3 | CR10584 | 9.5 | 4.5 |
| CR10396 | 49.2 | 2.3 | CR10491 | 35.3 | 8.3 | CR10585 | 24.5 | 7.5 |
| CR10397 | 34.6 | 4.7 | CR10492 | 16.5 | 2.6 | CR10586 | 1.8 | 0.7 |
| CR10398 | 53.9 | 2.6 | CR10493 | 44.8 | 5.1 | CR10587 | 11.8 | 6.3 |
| CR10399 | 39.5 | 3.5 | CR10494 | 46.5 | 4.4 | CR10588 | 9.1 | 2.9 |
| CR10400 | 45.2 | 3.4 | CR10495 | 45.3 | 3.2 | CR10589 | 10.8 | 4.7 |
| CR10401 | 44.2 | 2.4 | CR10496 | 45.6 | 4.8 | CR10590 | 5.7 | 2.3 |
| CR10402 | 43.9 | 2.2 | CR10497 | 48.9 | 3.6 | CR10591 | 26.9 | 10.4 |
| CR10403 | 33.4 | 1.4 | CR10498 | 44.9 | 3.5 | CR10592 | 18.4 | 6.0 |
| CR10404 | 35.5 | 3.4 | CR10499 | 41.1 | 2.4 | CR10593 | 9.3 | 7.9 |
| CR10405 | 55.5 | 3.4 | CR10500 | 34.5 | 6.2 | CR09641 | 13.2 | 1.7 |
| CR10406 | 43.2 | 2.1 | CR10501 | 31.6 | 2.6 | CR09630 | 14.3 | 6.7 |
| CR10407 | 24.0 | 8.6 | CR10502 | 23.7 | 3.1 | CR09633 | 7.4 | 1.6 |
| CR10408 | 21.1 | 5.2 | CR10503 | 21.3 | 4.1 | CR10594 | 5.5 | 3.3 |
| CR10409 | 38.8 | 8.0 | CR10504 | 20.6 | 2.1 | CR10595 | 7.0 | 1.5 |
| CR10410 | 17.7 | 4.8 | CR10505 | 27.6 | 1.5 | CR10596 | 6.0 | 0.9 |
| CR10411 | 20.1 | 6.8 | CR10506 | 14.7 | 3.9 | CR10597 | 4.6 | 0.2 |
| CR10412 | 19.1 | 4.2 | CR10507 | 20.9 | 1.0 | CR10598 | 2.2 | 0.4 |
| CR10413 | 18.0 | 4.5 | CR10508 | 15.6 | 4.2 | CR10599 | 5.4 | 1.8 |
| CR10879 | 39.8 | 4.8 | CR10973 | 26.3 | 6.4 | CR11067 | 10.9 | 3.0 |
| CR10880 | 12.1 | 2.7 | CR10974 | 14.2 | 7.3 | CR11068 | 3.0 | 1.0 |
| CR10881 | 5.7 | 0.2 | CR10975 | 4.8 | 0.5 | CR11069 | 0.5 | 0.1 |
| CR10882 | 42.4 | 12.3 | CR10976 | 27.5 | 4.1 | CR11070 | 8.8 | 3.5 |
| CR10883 | 38.9 | 8.2 | CR10977 | 29.1 | 9.8 | CR11071 | 9.8 | 0.8 |
| CR10884 | 50.8 | 11.5 | CR10978 | 26.1 | 11.7 | CR11072 | 7.8 | 2.5 |
| CR10885 | 44.9 | 9.2 | CR10979 | 24.6 | 2.3 | CR11073 | 14.2 | 4.2 |
| CR10886 | 45.5 | 8.8 | CR10980 | 31.5 | 3.4 | CR11074 | 14.7 | 5.4 |
| CR10887 | 34.8 | 8.0 | CR10981 | 33.3 | 2.6 | CR11075 | 15.6 | 3.7 |
| CR10888 | 44.4 | 9.7 | CR10982 | 40.0 | 10.8 | CR11076 | 14.8 | 7.1 |
| CR10889 | 49.4 | 13.9 | CR10983 | 41.7 | 5.9 | CR11077 | 14.6 | 0.7 |
| CR10890 | 3.9 | 1.6 | CR10984 | 8.3 | 9.8 | CR11078 | 0.3 | 0.2 |
| CR10891 | 27.4 | 12.8 | CR10985 | 18.4 | 1.7 | CR11079 | 11.8 | 1.2 |
| CR10892 | 19.3 | 3.2 | CR10986 | 11.3 | 3.8 | CR11080 | 7.2 | 1.3 |
| CR10893 | 26.5 | 5.3 | CR10987 | 12.5 | 3.6 | CR11081 | 4.7 | 0.9 |
| CR10894 | 25.6 | 3.0 | CR10988 | 13.8 | 4.9 | CR11082 | 4.9 | 0.9 |
| CR10895 | 28.3 | 4.3 | CR10989 | 21.9 | 9.4 | CR11083 | 10.3 | 3.3 |
| CR10896 | 27.2 | 10.0 | CR10990 | 16.9 | 3.3 | CR11084 | 4.9 | 0.6 |
| CR10897 | 4.5 | 2.4 | CR10991 | 2.8 | 0.5 | CR11085 | 0.3 | 0.1 |
| CR10898 | 24.3 | 5.1 | CR10992 | 19.1 | 1.4 | CR11086 | 6.8 | 0.2 |
| CR10899 | 25.4 | 6.5 | CR10993 | 36.9 | 4.3 | CR11087 | 11.6 | 0.9 |
| CR10900 | 4.1 | 0.4 | CR10994 | 4.4 | 4.3 | CR11088 | 0.1 | 0.0 |
| CR10901 | 6.8 | 2.0 | CR10995 | 8.0 | 7.0 | CR11089 | 1.5 | 0.4 |
| CR10902 | 17.7 | 4.0 | CR10996 | 11.8 | 8.4 | CR11090 | 5.1 | 0.8 |
| CR10903 | 15.8 | 5.9 | CR10997 | 11.5 | 1.9 | CR11091 | 3.9 | 0.6 |
| CR10904 | 22.8 | 1.1 | CR10998 | 14.7 | 5.3 | CR11092 | 5.1 | 1.1 |
| CR10905 | 5.2 | 1.6 | CR10999 | 2.6 | 0.9 | CR11093 | 1.1 | 0.6 |
| CR10906 | 14.8 | 0.9 | CR11000 | 17.3 | 4.6 | CR11094 | 5.1 | 3.6 |
| CR10907 | 27.3 | 6.9 | CR11001 | 20.2 | 6.4 | CR11095 | 7.7 | 5.5 |
| CR10908 | 14.5 | 5.2 | CR11002 | 13.4 | 4.3 | CR11096 | 11.3 | 4.6 |
| CR10909 | 19.7 | 2.8 | CR11003 | 23.4 | 2.3 | CR11097 | 10.5 | 4.3 |
| CR10910 | 1.8 | 0.3 | CR11004 | 0.9 | 0.2 | CR11098 | 0.3 | 0.1 |
| CR10911 | 5.7 | 0.6 | CR11005 | 7.1 | 0.2 | CR11099 | 0.6 | 0.3 |
| CR10912 | 15.1 | 2.1 | CR11006 | 19.6 | 3.5 | CR11100 | 11.3 | 1.0 |
| CR10913 | 2.3 | 0.9 | CR11007 | 0.7 | 0.2 | CR11101 | 0.3 | 0.1 |
| CR10914 | 24.4 | 5.5 | CR11008 | 14.7 | 6.3 | CR11102 | 7.6 | 1.3 |
| CR10915 | 6.6 | 2.2 | CR11009 | 2.5 | 0.6 | CR11103 | 1.0 | 0.3 |
| CR10916 | 4.0 | 1.6 | CR11010 | 1.6 | 0.4 | CR11104 | 0.4 | 0.1 |
| CR10917 | 18.2 | 4.0 | CR11011 | 21.7 | 6.5 | CR11105 | 3.8 | 0.8 |
| CR10918 | 25.3 | 0.7 | CR11012 | 12.8 | 2.6 | CR11106 | 2.0 | 0.7 |
| CR10919 | 18.9 | 5.9 | CR11013 | 12.9 | 1.5 | CR11107 | 3.9 | 0.3 |
| CR10920 | 22.0 | 4.1 | CR11014 | 19.8 | 1.8 | CR11108 | 11.3 | 1.2 |
| CR10921 | 16.3 | 3.6 | CR11015 | 9.3 | 2.4 | CR11109 | 6.2 | 1.5 |
| CR10922 | 2.8 | 0.6 | CR11016 | 2.6 | 0.9 | CR11110 | 0.7 | 0.1 |
| CR10923 | 24.7 | 3.5 | CR11017 | 12.7 | 1.7 | CR11111 | 7.8 | 1.3 |
| CR10924 | 23.8 | 5.1 | CR11018 | 10.9 | 3.8 | CR11112 | 6.7 | 2.5 |
| CR10925 | 16.6 | 5.1 | CR11019 | 16.0 | 4.0 | CR11113 | 13.2 | 3.3 |
| CR10926 | 2.2 | 0.2 | CR11020 | 0.8 | 0.1 | CR11114 | 0.2 | 0.1 |
| CR10927 | 17.2 | 5.4 | CR11021 | 6.4 | 0.8 | CR11115 | 5.5 | 0.9 |
| CR10928 | 18.7 | 5.7 | CR11022 | 17.3 | 4.1 | CR11116 | 4.1 | 0.8 |
| CR10929 | 20.5 | 3.7 | CR11023 | 22.6 | 7.3 | CR11117 | 2.1 | 0.3 |
| CR10930 | 22.1 | 1.8 | CR11024 | 12.1 | 4.8 | CR11118 | 4.8 | 1.1 |
| CR10931 | 3.4 | 0.9 | CR11025 | 1.1 | 0.6 | CR11119 | 0.1 | 0.1 |
| CR10932 | 1.3 | 0.2 | CR11026 | 1.2 | 0.4 | CR11120 | 0.2 | 0.0 |
| CR10933 | 18.0 | 2.8 | CR11027 | 11.2 | 3.0 | CR11121 | 3.9 | 0.8 |
| CR10934 | 10.8 | 0.6 | CR11028 | 8.7 | 3.2 | CR11122 | 3.4 | 0.3 |
| CR10935 | 1.4 | 0.2 | CR11029 | 1.5 | 0.3 | CR11123 | 0.2 | 0.1 |
| CR10936 | 2.9 | 0.5 | CR11030 | 2.2 | 0.5 | CR11124 | 2.2 | 0.2 |
| CR10937 | 13.9 | 0.9 | CR11031 | 18.5 | 6.7 | CR11125 | 5.8 | 0.9 |
| CR10938 | 1.5 | 0.3 | CR11032 | 0.7 | 0.1 | CR11126 | 0.1 | 0.1 |
| CR10939 | 20.1 | 0.7 | CR11033 | 13.2 | 2.4 | CR11127 | 3.8 | 0.8 |
| CR10940 | 14.8 | 3.3 | CR11034 | 12.8 | 3.3 | CR11128 | 5.6 | 0.8 |
| CR10941 | 18.9 | 5.8 | CR11035 | 20.6 | 0.6 | CR11129 | 7.3 | 1.3 |
| CR10942 | 22.2 | 0.5 | CR11036 | 24.9 | 4.5 | CR11130 | 21.2 | 8.2 |
| CR10943 | 22.5 | 4.2 | CR11037 | 19.3 | 4.1 | CR11131 | 9.2 | 3.7 |
| CR10944 | 1.0 | 0.2 | CR11038 | 0.5 | 0.2 | CR11132 | 0.1 | 0.1 |
| CR10945 | 1.6 | 0.6 | CR11039 | 1.0 | 0.5 | CR11133 | 0.3 | 0.1 |
| CR10946 | 16.7 | 0.8 | CR11040 | 19.4 | 0.2 | CR11134 | 7.0 | 1.6 |
| CR10947 | 2.5 | 0.8 | CR11041 | 1.5 | 0.4 | CR11135 | 0.4 | 0.1 |
| CR10948 | 11.6 | 2.6 | CR11042 | 5.0 | 0.7 | CR11136 | 5.8 | 1.5 |
| CR10949 | 15.1 | 3.3 | CR11043 | 20.4 | 3.1 | CR11137 | 5.5 | 1.2 |
| CR10950 | 2.1 | 0.4 | CR11044 | 0.8 | 0.3 | CR11138 | 0.1 | 0.1 |
| CR10951 | 15.3 | 3.1 | CR11045 | 12.2 | 4.3 | CR11139 | 5.8 | 0.7 |
| CR10952 | 1.1 | 0.2 | CR11046 | 1.5 | 0.2 | CR11140 | 0.2 | 0.1 |
| CR10953 | 42.1 | 3.7 | CR11047 | 35.8 | 8.0 | CR11141 | 11.8 | 3.3 |
| CR10954 | 3.6 | 1.0 | CR11048 | 3.2 | 0.3 | CR11142 | 0.4 | 0.1 |
| CR10955 | 5.0 | 2.1 | CR11049 | 3.6 | 1.5 | CR11143 | 5.3 | 2.1 |
| CR10956 | 1.0 | 0.3 | CR11050 | 0.6 | 0.2 | CR11144 | 0.1 | 0.0 |
| CR10957 | 16.2 | 5.9 | CR11051 | 6.0 | 1.0 | CR11145 | 4.3 | 4.0 |
| CR10958 | 33.3 | 6.7 | CR11052 | 23.5 | 3.4 | CR11146 | 15.3 | 2.2 |
| CR10959 | 38.1 | 9.3 | CR11053 | 24.4 | 7.9 | CR11147 | 7.3 | 2.2 |
| CR10960 | 2.1 | 0.8 | CR11054 | 0.5 | 0.1 | CR11148 | 0.2 | 0.1 |
| CR10961 | 17.7 | 4.7 | CR11055 | 35.3 | 7.6 | CR11149 | 32.5 | 6.9 |
| CR10962 | 8.5 | 3.9 | CR11056 | 7.4 | 1.4 | CR11150 | 1.5 | 0.4 |
| CR10963 | 3.2 | 0.9 | CR11057 | 3.1 | 1.2 | CR11151 | 0.7 | 0.2 |
| CR10964 | 45.6 | 11.5 | CR11058 | 26.3 | 2.9 | CR09583 | 13.7 | 5.0 |
| CR10965 | 39.2 | 18.0 | CR11059 | 15.6 | 0.6 | CR09586 | 3.7 | 0.7 |
| CR10966 | 21.3 | 8.8 | CR11060 | 8.0 | 1.5 | CR09594 | 5.6 | 2.9 |
| CR10967 | 2.5 | 0.4 | CR11061 | 1.6 | 0.3 | CR11152 | 0.1 | 0.1 |
| CR10968 | 17.2 | 5.8 | CR11062 | 13.6 | 8.0 | CR11153 | 7.4 | 1.1 |
| CR10969 | 17.3 | 3.9 | CR11063 | 13.0 | 2.0 | CR11154 | 5.2 | 1.0 |
| CR10970 | 16.0 | 5.2 | CR11064 | 9.1 | 2.7 | CR11155 | 2.2 | 0.3 |
| CR10971 | 2.1 | 0.2 | CR11065 | 0.7 | 0.1 | CR11156 | 0.1 | 0.1 |
| CR10972 | 3.4 | 0.3 | CR11066 | 2.5 | 0.7 | CR11157 | 0.6 | 0.1 |
| CR11440 | 38.4 | 9.5 | CR11534 | 59.1 | 7.6 | CR11628 | 16.4 | 0.9 |
| CR11441 | 20.6 | 5.0 | CR11535 | 32.2 | 5.4 | CR11629 | 11.1 | 0.9 |
| CR11442 | 27.2 | 3.7 | CR11536 | 26.8 | 1.9 | CR11630 | 18.6 | 0.8 |
| CR11443 | 16.5 | 5.0 | CR11537 | 16.8 | 6.0 | CR11631 | 3.7 | 1.3 |
| CR11444 | 23.9 | 2.2 | CR11538 | 33.6 | 14.8 | CR11632 | 10.2 | 1.8 |
| CR11445 | 13.8 | 3.4 | CR11539 | 25.0 | 3.5 | CR11633 | 6.7 | 2.2 |
| CR11446 | 31.4 | 0.8 | CR11540 | 29.4 | 6.0 | CR11634 | 11.0 | 1.4 |
| CR11447 | 25.2 | 1.6 | CR11541 | 23.4 | 1.5 | CR11635 | 8.5 | 1.2 |
| CR11448 | 18.1 | 4.7 | CR11542 | 18.0 | 2.3 | CR11636 | 5.6 | 0.4 |
| CR11449 | 12.6 | 4.6 | CR11543 | 27.9 | 0.9 | CR11637 | 13.7 | 2.3 |
| CR11450 | 23.0 | 3.5 | CR11544 | 30.4 | 2.0 | CR11638 | 12.0 | 1.5 |
| CR11451 | 21.4 | 2.8 | CR11545 | 22.7 | 6.9 | CR11639 | 20.6 | 7.8 |
| CR11452 | 18.7 | 1.5 | CR11546 | 38.6 | 19.0 | CR11640 | 9.4 | 3.8 |
| CR11453 | 11.8 | 2.7 | CR11547 | 25.5 | 12.8 | CR11641 | 4.4 | 0.6 |
| CR11454 | 11.5 | 5.4 | CR11548 | 11.2 | 3.0 | CR11642 | 5.1 | 1.5 |
| CR11455 | 12.1 | 3.5 | CR11549 | 15.2 | 9.9 | CR11643 | 2.3 | 0.7 |
| CR11456 | 17.0 | 0.4 | CR11550 | 11.9 | 8.4 | CR11644 | 9.0 | 2.5 |
| CR11457 | 6.4 | 0.3 | CR11551 | 6.9 | 1.2 | CR11645 | 1.4 | 0.6 |
| CR11458 | 4.8 | 0.2 | CR11552 | 7.0 | 3.6 | CR11646 | 1.4 | 0.5 |
| CR11459 | 9.8 | 0.7 | CR11553 | 12.2 | 3.1 | CR11647 | 10.3 | 1.8 |
| CR11460 | 12.3 | 2.0 | CR11554 | 14.4 | 4.9 | CR11648 | 7.2 | 1.8 |
| CR11461 | 13.0 | 1.8 | CR11555 | 25.5 | 4.7 | CR11649 | 13.6 | 3.5 |
| CR11462 | 14.3 | 2.0 | CR11556 | 14.6 | 5.9 | CR11650 | 6.3 | 0.9 |
| CR11463 | 11.4 | 1.1 | CR11557 | 6.1 | 1.1 | CR11651 | 3.5 | 0.4 |
| CR11464 | 8.7 | 1.4 | CR11558 | 8.5 | 4.4 | CR11652 | 4.7 | 1.6 |
| CR11465 | 5.1 | 0.6 | CR11559 | 5.0 | 0.9 | CR11653 | 1.3 | 0.2 |
| CR11466 | 8.0 | 2.3 | CR11560 | 8.6 | 2.9 | CR11654 | 2.7 | 0.7 |
| CR11467 | 9.2 | 1.4 | CR11561 | 14.4 | 7.7 | CR11655 | 3.0 | 0.9 |
| CR11468 | 12.0 | 0.3 | CR11562 | 5.6 | 1.1 | CR11656 | 2.3 | 0.6 |
| CR11469 | 6.8 | 1.6 | CR11563 | 6.0 | 4.4 | CR11657 | 1.1 | 0.3 |
| CR11470 | 5.1 | 0.1 | CR11564 | 9.7 | 5.2 | CR11658 | 8.0 | 2.5 |
| CR11471 | 11.5 | 3.5 | CR11565 | 6.9 | 2.0 | CR11659 | 4.5 | 1.7 |
| CR11472 | 16.9 | 6.4 | CR11566 | 12.7 | 3.9 | CR11660 | 2.0 | 0.5 |
| CR11473 | 5.7 | 0.3 | CR11567 | 11.5 | 12.4 | CR11661 | 2.7 | 0.7 |
| CR11474 | 8.0 | 2.1 | CR11568 | 4.2 | 1.8 | CR11662 | 2.8 | 0.5 |
| CR11475 | 5.2 | 1.1 | CR11569 | 9.4 | 3.2 | CR11663 | 1.8 | 0.7 |
| CR11476 | 3.8 | 0.4 | CR11570 | 2.5 | 0.4 | CR11664 | 1.1 | 0.3 |
| CR11477 | 5.4 | 0.6 | CR11571 | 3.6 | 2.0 | CR11665 | 1.2 | 0.4 |
| CR11478 | 4.8 | 0.0 | CR11572 | 10.0 | 4.0 | CR11666 | 3.3 | 0.4 |
| CR11479 | 5.7 | 0.1 | CR11573 | 2.3 | 1.0 | CR11667 | 0.6 | 0.1 |
| CR11480 | 7.1 | 0.8 | CR11574 | 3.2 | 1.4 | CR11668 | 2.2 | 0.5 |
| CR11481 | 7.3 | 0.1 | CR11575 | 12.0 | 2.4 | CR11669 | 7.0 | 1.8 |
| CR11482 | 13.2 | 1.3 | CR11576 | 4.7 | 1.4 | CR11670 | 2.6 | 0.4 |
| CR11483 | 5.5 | 0.2 | CR11577 | 6.9 | 4.0 | CR11671 | 1.3 | 0.5 |
| CR11484 | 4.4 | 1.1 | CR11578 | 4.3 | 2.4 | CR11672 | 1.1 | 0.3 |
| CR11485 | 2.4 | 0.3 | CR11579 | 3.1 | 0.7 | CR11673 | 2.3 | 0.5 |
| CR11486 | 4.2 | 0.1 | CR11580 | 5.7 | 1.5 | CR11674 | 1.1 | 0.3 |
| CR11487 | 8.8 | 0.6 | CR11581 | 10.4 | 3.3 | CR11675 | 2.3 | 0.6 |
| CR11488 | 6.3 | 1.7 | CR11582 | 5.3 | 2.9 | CR11676 | 2.1 | 0.7 |
| CR11489 | 1.8 | 0.1 | CR11583 | 1.4 | 0.3 | CR11677 | 0.5 | 0.1 |
| CR11490 | 4.4 | 0.1 | CR11584 | 4.3 | 0.9 | CR11678 | 3.1 | 0.5 |
| CR11491 | 6.9 | 1.8 | CR11585 | 4.7 | 2.1 | CR11679 | 2.1 | 0.4 |
| CR11492 | 6.3 | 0.3 | CR11586 | 11.7 | 2.5 | CR11680 | 8.5 | 2.8 |
| CR11493 | 10.5 | 2.5 | CR11587 | 18.6 | 19.7 | CR11681 | 3.0 | 0.7 |
| CR11494 | 2.5 | 0.4 | CR11588 | 4.2 | 1.3 | CR11682 | 1.0 | 0.4 |
| CR11495 | 3.4 | 0.4 | CR11589 | 4.3 | 1.0 | CR11683 | 1.4 | 0.1 |
| CR11496 | 4.8 | 0.3 | CR11590 | 7.0 | 1.7 | CR11684 | 1.7 | 0.2 |
| CR11497 | 2.6 | 0.7 | CR11591 | 2.5 | 1.0 | CR11685 | 1.2 | 0.1 |
| CR11498 | 3.7 | 0.6 | CR11592 | 4.6 | 1.0 | CR11686 | 1.5 | 0.4 |
| CR11499 | 2.3 | 0.1 | CR11593 | 1.1 | 0.3 | CR11687 | 0.3 | 0.1 |
| CR11500 | 6.6 | 0.5 | CR11594 | 2.6 | 0.1 | CR11688 | 1.3 | 0.3 |
| CR11501 | 3.3 | 0.0 | CR11595 | 2.0 | 0.9 | CR11689 | 0.8 | 0.1 |
| CR11502 | 5.6 | 1.1 | CR11596 | 4.7 | 1.1 | CR11690 | 5.1 | 3.3 |
| CR11503 | 4.2 | 0.2 | CR11597 | 4.2 | 0.8 | CR11691 | 1.1 | 0.1 |
| CR11504 | 2.9 | 0.1 | CR11598 | 4.0 | 1.9 | CR11692 | 1.0 | 0.3 |
| CR11505 | 3.1 | 0.4 | CR11599 | 1.3 | 0.1 | CR11693 | 0.3 | 0.1 |
| CR11506 | 2.4 | 0.2 | CR11600 | 1.7 | 0.2 | CR11694 | 0.7 | 0.1 |
| CR11507 | 3.8 | 0.8 | CR11601 | 3.5 | 1.2 | CR11695 | 0.7 | 0.2 |
| CR11508 | 2.3 | 0.4 | CR11602 | 1.9 | 0.3 | CR11696 | 0.7 | 0.3 |
| CR11509 | 2.6 | 0.0 | CR11603 | 2.0 | 0.4 | CR11697 | 0.9 | 0.2 |
| CR11510 | 1.7 | 0.6 | CR11604 | 1.2 | 0.2 | CR11698 | 0.7 | 0.2 |
| CR11511 | 3.1 | 0.1 | CR11605 | 1.8 | 0.5 | CR11699 | 0.6 | 0.1 |
| CR11512 | 3.1 | 0.8 | CR11606 | 1.7 | 0.3 | CR11700 | 1.1 | 0.4 |
| CR11513 | 4.1 | 0.6 | CR11607 | 1.7 | 0.3 | CR11701 | 5.7 | 4.6 |
| CR11514 | 3.8 | 0.2 | CR11608 | 6.1 | 1.0 | CR11702 | 1.7 | 0.8 |
| CR11515 | 3.7 | 0.7 | CR11609 | 2.9 | 0.7 | CR11703 | 3.3 | 0.2 |
| CR11516 | 4.8 | 0.5 | CR11610 | 14.3 | 19.7 | CR11704 | 1.5 | 0.4 |
| CR11517 | 7.8 | 4.5 | CR11611 | 2.6 | 2.7 | CR11705 | 1.3 | 0.2 |
| CR11518 | 5.1 | 0.1 | CR11612 | 2.3 | 0.6 | CR11706 | 1.2 | 0.2 |
| CR11519 | 5.9 | 0.3 | CR11613 | 2.5 | 0.6 | CR11707 | 1.6 | 0.4 |
| CR11520 | 2.9 | 0.3 | CR11614 | 5.0 | 5.8 | CR11708 | 1.2 | 0.5 |
| CR11521 | 3.5 | 0.1 | CR11615 | 2.2 | 0.5 | CR11709 | 3.7 | 1.7 |
| CR11522 | 3.6 | 0.5 | CR11616 | 2.7 | 1.4 | CR11710 | 2.8 | 1.4 |
| CR11523 | 4.5 | 0.1 | CR11617 | 12.0 | 15.0 | CR11711 | 3.0 | 1.2 |
| CR11524 | 4.2 | 1.4 | CR11618 | 3.5 | 0.1 | CR11712 | 3.2 | 0.8 |
| CR11525 | 2.1 | 0.3 | CR11619 | 3.9 | 2.2 | CR11713 | 1.4 | 0.3 |
| CR11526 | 22.3 | 2.8 | CR11620 | 15.9 | 8.9 | CR11714 | 3.1 | 0.2 |
| CR11527 | 13.7 | 1.1 | CR11621 | 6.2 | 4.3 | CR11715 | 1.7 | 0.3 |
| CR11528 | 15.4 | 0.4 | CR11622 | 11.7 | 7.2 | CR11716 | 2.4 | 0.6 |
| CR11529 | 12.1 | 1.1 | CR11623 | 9.0 | 7.8 | CR11717 | 4.3 | 2.0 |
| CR11530 | 5.2 | 0.9 | CR11624 | 6.6 | 1.4 | CR11718 | 2.9 | 0.8 |
| CR11531 | 7.7 | 0.2 | CR11625 | 4.8 | 1.7 | CR11719 | 1.7 | 0.6 |
| CR11532 | 5.7 | 0.4 | CR11626 | 2.5 | 0.5 | CR11720 | 0.5 | 0.1 |
| CR11533 | 6.5 | 0.9 | CR11627 | 3.7 | 1.5 | CR11721 | 1.2 | 0.2 |

### Example 4

The impacts of chemical modification type and position within the guide domain were evaluated in an editing screen of modified sgRNAs. The screen assayed guides modified with 2′F, 2′OMe and PS. The complete pattern set was applied to the nucleobase sequence of three guides. Test modification patterns in this example were applied to three guide domain nucleobase sequences, specifically those described in Table 1 for G000486, G000502, or G000415. Guide domain modification patterns were assayed either the conserved region described in SEQ ID No. 695 or in a short-sgRNA format using the conserved region described in SEQ ID No. 253. The final data set contained 270 distinct guides and 45 unique modification patterns in the guide domain.

Rows 1-12 in Tables 9-12 show test guides assessed for editing efficacy with 2′OMe, 2′F, PS, and 2′H modifications at positions 5, 12, and 15. Rows 13-19 in Tables 9-12 show editing data for variants that substitute 2′F single modification for 2′F+PS modifications at positions 8-10. Rows 20-27 in Tables 9-12 show editing data for variants that substitute 2′F for PS at positions 4-20.

Editing was assayed in PCH and PHH cells as described in Example 1 with the following modifications. Cells were counted and plated at a density of 30,000-35,000 cells/well for PHH, and 40,000-60,000 cells/well for PCH Transfections used pre-mixed lipid formulations in which the lipid components were reconstituted in 100% ethanol at a molar ratio of 50% Lipid A, 9% DSPC, 38% cholesterol, and 3% PEG2k-DMG. The lipid mixture was then mixed with RNA cargos (e.g., Cas9 mRNA and gRNA) at a lipid amine to RNA phosphate (N:P) molar ratio of about 6.0. Transfections were performed with a final concentration of 100 nM gRNA, 3% cyno serum, and 50 ng Cas9 mRNA per well. Cells were incubated for approximately 48 hours before cell lysis and NGS analysis. The experiment was performed in duplicate. The editing results are described in Tables 9-12. Each row represents a single modification pattern design with the same conserved region. Rows 46-48 are controls.

The data were analyzed to estimate the impact of several variables, including whether the guide was or was not a short-sgRNA, variable region modification pattern, and individual modification position, where possible. Short-sgRNA guides were significantly more active than non-short-sgRNA guides in PCH (all sites) and PHH (G502 variants). In PCH, short-sgRNA guides added an additional 14% over equivalent non-short-sgRNAs.

Many of the modification patterns in this study were designed to incorporate and further test well tolerated modifications into highly modified gRNA molecules. In general this was successful; nearly all patterns showed similar activity to controls overall. A number of individual positions were also tested in this study. Position 5, 12 and 15 were modified individually. Position 5 was highly tolerant of modification. Position 12 was tolerant of PS, 2′-F and 2′-OMe, but was significantly sensitive to 2′-H modification (reduction of editing percentage by 7.5, p<0.00002). Position 15 was tolerant of 2′-H modification, but as in other work presented here, highly sensitive to 2′F and 2′OMe (p<10⁻¹³).

TABLE 9
Average Editing % in PHH cells
PHH

| | G000502 | | | G000486 | | | G000415 | | |
|---|---|---|---|---|---|---|---|---|---|
| Row | Variants | % Edit | SD | Variants | % Edit | SD | Variants | % Edit | SD |
| 1 | G014462 | 21.7% | 0.2% | G014507 | 57.0% | 0.3% | G014554 | 57.2% | 2.1% |
| 2 | G014463 | 21.4% | 1.7% | G014508 | 57.6% | 0.2% | G014555 | 56.0% | 0.4% |
| 3 | G014464 | 28.4% | 2.9% | G014509 | 58.6% | 2.5% | G014556 | 54.4% | 5.2% |
| 4 | G014465 | 23.7% | 1.0% | G014510 | 52.5% | 0.6% | G014557 | 54.9% | 1.3% |
| 5 | G014466 | 22.8% | 0.1% | G014511 | 54.4% | 0.6% | G014558 | 61.0% | 4.1% |
| 6 | G014467 | 27.9% | 2.3% | G014512 | 62.1% | 2.4% | G014559 | 63.4% | 0.1% |
| 7 | G014468 | 21.5% | 1.3% | G014513 | 57.6% | 0.7% | G014560 | 55.6% | 0.8% |
| 8 | G014469 | 12.6% | 0.3% | G014514 | 52.8% | 1.9% | G014561 | 39.0% | 0.6% |
| 9 | G014470 | 3.4% | 1.1% | G014515 | 35.5% | 1.2% | G014562 | 32.8% | 6.8% |
| 10 | G014471 | 9.1% | 0.6% | G014516 | 48.6% | 1.0% | G014563 | 33.0% | 6.7% |
| 11 | G014472 | 21.2% | 0.6% | G014517 | 55.5% | 0.1% | G014564 | 63.1% | 0.6% |
| 12 | G014473 | 18.0% | 0.9% | G014518 | 54.2% | 0.1% | G014565 | 55.2% | 2.5% |
| 13 | G014474 | 25.4% | 3.7% | G014519 | 52.3% | 0.7% | G014566 | 64.9% | 6.8% |
| 14 | G014475 | 31.5% | 3.3% | G014520 | 60.3% | 0.1% | G014567 | 55.2% | 0.3% |
| 15 | G014476 | 26.3% | 4.5% | G014521 | 56.5% | 0.4% | G014568 | 54.5% | 5.3% |
| 16 | G014477 | 28.2% | 2.1% | G014522 | 56.6% | 5.4% | G014569 | 59.7% | 0.7% |
| 17 | G014478 | 30.6% | 1.5% | G014523 | 64.7% | 0.2% | G014570 | 50.4% | 3.5% |
| 18 | G014479 | 32.3% | 0.3% | G014524 | 59.9% | 7.3% | G014571 | 56.7% | 3.3% |
| 19 | G014480 | 37.9% | 0.8% | G014525 | 65.1% | 2.1% | G014572 | 49.6% | 3.2% |
| 20 | G014481 | 27.8% | 0.4% | G014526 | 53.6% | 0.9% | G014573 | 44.0% | 0.4% |
| 21 | G014482 | 26.0% | 0.4% | G014527 | 54.1% | 1.2% | G014574 | 59.3% | 4.7% |
| 22 | G014483 | 23.8% | 3.3% | G014528 | 53.3% | 1.4% | G014575 | 50.8% | 7.6% |
| 23 | G014484 | 21.6% | 2.5% | G014529 | 47.6% | 0.5% | G014576 | 45.8% | 13.8% |
| 24 | G014485 | 21.6% | 3.7% | G014530 | 43.9% | 4.6% | G014577 | 56.1% | 5.2% |
| 25 | G014486 | 24.2% | 2.8% | G014531 | 52.8% | 4.6% | G014578 | 61.3% | 3.6% |
| 26 | G014487 | 25.9% | 3.3% | G014532 | 51.2% | 1.3% | G014579 | 48.9% | 7.8% |
| 27 | G014488 | 22.0% | 4.1% | G014533 | 49.9% | 5.4% | G014580 | 50.2% | 7.1% |
| 28 | G014489 | 34.7% | 3.0% | G014534 | 67.2% | 5.1% | G014581 | 54.2% | 5.5% |
| 29 | G014490 | 38.1% | 4.4% | G014535 | 65.6% | 4.6% | G014582 | 62.0% | 2.5% |
| 30 | G014491 | 34.8% | 1.8% | G014536 | 69.5% | 5.9% | G014583 | 51.8% | 6.6% |
| 31 | G014492 | 23.5% | 1.1% | G014537 | 66.8% | 2.0% | G014584 | 54.4% | 2.4% |
| 32 | G014493 | 38.9% | 6.1% | G014538 | 61.4% | 12.4% | G014585 | 53.7% | 15.2% |
| 33 | G014494 | 38.1% | 3.7% | G014539 | 61.0% | 3.7% | G014586 | 56.1% | 4.2% |
| 34 | G014495 | 33.4% | 1.3% | G014540 | 63.7% | 5.9% | G014587 | 60.0% | 5.9% |
| 35 | G014496 | 42.9% | 2.5% | G014541 | 64.0% | 4.2% | G014588 | 52.5% | 2.9% |
| 36 | G014497 | 39.0% | 3.7% | G014542 | 51.2% | 4.3% | G014589 | 56.8% | 4.3% |
| 37 | G014498 | 37.0% | 3.3% | G014543 | 70.7% | 2.7% | G014590 | 49.8% | 7.0% |
| 38 | G014499 | 34.9% | 0.3% | G014544 | 36.7% | 0.1% | G014591 | 50.3% | 2.3% |
| 39 | G014500 | 28.2% | 1.7% | G014545 | 52.8% | 0.0% | G014592 | 49.8% | 5.8% |
| 40 | G014501 | 30.5% | 2.0% | G014546 | 56.6% | 3.8% | G014593 | 53.4% | 4.5% |
| 41 | G014502 | 33.3% | 4.2% | G014547 | 53.4% | 3.9% | G014594 | 51.8% | 0.7% |
| 42 | G014503 | 28.8% | 2.5% | G014548 | 36.3% | 1.3% | G014595 | 55.6% | 2.5% |
| 43 | G014504 | 5.9% | 0.5% | G014549 | 64.6% | 2.3% | G014596 | 39.2% | 4.0% |
| 44 | G014505 | 0.2% | 0.0% | G014550 | 16.0% | 3.3% | G014597 | 0.4% | 0.3% |
| 45 | G014506 | 11.0% | 0.1% | G014551 | 49.6% | 1.6% | G014598 | 38.5% | 0.9% |
| 46 | G000502 | 51.4% | 3.3% | G000502 | 51.4% | 3.3% | G000502 | 53.5% | 3.1% |
| 47 | G010039 | 29.4% | 0.1% | G010039 | 29.4% | 0.1% | G010039 | 22.6% | 3.0% |
| 48 | G012401 | 56.9% | 0.1% | G012401 | 56.9% | 0.1% | G012401 | 52.2% | 4.7% |

TABLE 10
Average Editing % in PCH cells
PCH

| | G000502 | | | G000486 | | | G000415 | | |
|---|---|---|---|---|---|---|---|---|---|
| Row | Variants | % Edit | SD | Variants | % Edit | SD | Variants | % Edit | SD |
| 1 | G014462 | 54.6% | 2.5% | G014507 | 4.6% | 0.1% | G014554 | 46.8% | 8.1% |
| 2 | G014463 | 43.9% | 2.1% | G014508 | 5.2% | 0.7% | G014555 | 38.2% | 2.3% |
| 3 | G014464 | 50.6% | 5.1% | G014509 | 4.1% | 1.2% | G014556 | 34.1% | 2.1% |
| 4 | G014465 | 49.1% | 3.3% | G014510 | 3.2% | 1.8% | G014557 | 34.9% | 7.1% |
| 5 | G014466 | 51.3% | 9.2% | G014511 | 3.1% | 1.1% | G014558 | 37.5% | 4.3% |
| 6 | G014467 | 49.4% | 9.7% | G014512 | 6.2% | 2.0% | G014559 | 36.5% | 0.9% |
| 7 | G014468 | 48.2% | 4.7% | G014513 | 6.3% | 2.3% | G014560 | 36.1% | 7.0% |
| 8 | G014469 | 40.4% | 3.8% | G014514 | 2.5% | 1.1% | G014561 | 28.7% | 3.1% |
| 9 | G014470 | 31.2% | 7.8% | G014515 | 0.6% | 0.3% | G014562 | 20.7% | 1.4% |
| 10 | G014471 | 37.1% | 8.6% | G014516 | 1.8% | 0.5% | G014563 | 16.5% | 1.5% |
| 11 | G014472 | 50.3% | 12.4% | G014517 | 5.6% | 2.5% | G014564 | 45.1% | 3.0% |
| 12 | G014473 | 55.1% | 9.3% | G014518 | 7.1% | 0.9% | G014565 | 42.8% | 1.1% |
| 13 | G014474 | 47.6% | 6.1% | G014519 | 4.5% | 0.4% | G014566 | 46.8% | 2.9% |
| 14 | G014475 | 43.8% | 1.8% | G014520 | 5.5% | 1.7% | G014567 | 35.5% | 1.8% |
| 15 | G014476 | 38.2% | 10.0% | G014521 | 4.7% | 1.4% | G014568 | 32.7% | 4.9% |
| 16 | G014477 | 47.1% | 7.2% | G014522 | 5.7% | 3.5% | G014569 | 38.8% | 0.4% |
| 17 | G014478 | 45.9% | 4.4% | G014523 | 5.3% | 1.6% | G014570 | 28.9% | 1.8% |
| 18 | G014479 | 49.3% | 6.5% | G014524 | 7.1% | 2.1% | G014571 | 31.1% | 0.8% |
| 19 | G014480 | 49.3% | 11.9% | G014525 | 8.7% | 5.0% | G014572 | 28.0% | 1.6% |
| 20 | G014481 | 43.9% | 8.9% | G014526 | 6.3% | 1.3% | G014573 | 15.6% | 0.4% |
| 21 | G014482 | 43.6% | 4.2% | G014527 | 5.9% | 1.2% | G014574 | 30.6% | 4.1% |
| 22 | G014483 | 45.4% | 4.7% | G014528 | 5.4% | 0.4% | G014575 | 18.8% | 2.7% |
| 23 | G014484 | 43.6% | 6.7% | G014529 | 6.5% | 2.4% | G014576 | 33.2% | 9.5% |
| 24 | G014485 | 38.5% | 3.5% | G014530 | 6.8% | 1.6% | G014577 | 34.3% | 2.1% |
| 25 | G014486 | 32.0% | 0.3% | G014531 | 6.1% | 1.7% | G014578 | 44.1% | 7.9% |
| 26 | G014487 | 42.5% | 4.0% | G014532 | 5.8% | 0.7% | G014579 | 28.8% | 5.4% |
| 27 | G014488 | 32.3% | 7.6% | G014533 | 4.6% | 1.1% | G014580 | 31.9% | 4.7% |
| 28 | G014489 | 40.2% | 7.3% | G014534 | 9.0% | 0.6% | G014581 | 26.7% | 4.8% |
| 29 | G014490 | 35.3% | 7.7% | G014535 | 7.0% | 2.6% | G014582 | 32.4% | 1.5% |
| 30 | G014491 | 48.9% | 7.1% | G014536 | 7.9% | 1.3% | G014583 | 28.8% | 4.1% |
| 31 | G014492 | 38.8% | 9.2% | G014537 | 5.8% | 1.8% | G014584 | 29.5% | 1.6% |
| 32 | G014493 | 50.4% | 8.5% | G014538 | 7.6% | 1.9% | G014585 | 23.3% | 5.4% |
| 33 | G014494 | 50.5% | 6.7% | G014539 | 7.6% | 0.7% | G014586 | 27.2% | 3.1% |
| 34 | G014495 | 57.7% | 0.7% | G014540 | 7.4% | 1.8% | G014587 | 33.1% | 1.6% |
| 35 | G014496 | 49.3% | 1.9% | G014541 | 5.0% | 0.6% | G014588 | 34.5% | 10.9% |
| 36 | G014497 | 36.5% | 6.9% | G014542 | 16.7% | 1.8% | G014589 | 25.2% | 1.5% |
| 37 | G014498 | 39.2% | 7.5% | G014543 | 7.0% | 1.6% | G014590 | 21.9% | 1.6% |
| 38 | G014499 | 46.7% | 4.5% | G014544 | 1.8% | 0.6% | G014591 | 26.8% | 4.0% |
| 39 | G014500 | 37.1% | 10.0% | G014545 | 5.2% | 1.5% | G014592 | 19.9% | 1.2% |
| 40 | G014501 | 40.1% | 5.7% | G014546 | 4.9% | 0.7% | G014593 | 26.0% | 3.6% |
| 41 | G014502 | 43.1% | 3.7% | G014547 | 2.5% | 0.5% | G014594 | 22.8% | 1.8% |
| 42 | G014503 | 52.5% | 4.7% | G014548 | 1.7% | 0.8% | G014595 | 22.6% | 5.4% |
| 43 | G014504 | 13.1% | 8.0% | G014549 | 3.1% | 0.1% | G014596 | 20.6% | 7.4% |
| 44 | G014505 | 3.0% | 0.3% | G014550 | 0.6% | 0.0% | G014597 | 0.9% | 0.8% |
| 45 | G014506 | 44.1% | 2.0% | G014551 | 1.9% | 1.1% | G014598 | 24.6% | 9.1% |
| 46 | G000502 | 55.8% | 18.1% | G000502 | 55.8% | 18.1% | G000502 | 47.9% | 0.1% |
| 47 | G010039 | 44.4% | 8.0% | G010039 | 44.4% | 8.0% | G010039 | 39.4% | 10.6% |
| 48 | G012401 | 65.4% | 9.0% | G012401 | 65.4% | 9.0% | G012401 | 53.3% | 4.5% |

TABLE 11
Average Editing % in PHH cells for short-sgRNA guides
PHH - short-sgRNA

| | G000502 | | | G000486 | | | G000415 | | |
|---|---|---|---|---|---|---|---|---|---|
| Row | Variants | % Edit | SD | Variants | % Edit | SD | Variants | % Edit | SD |
| 1 | G014644 | 57.1% | 5.9% | G014689 | 43.4% | 0.4% | G014734 | 57.5% | 1.1% |
| 2 | G014645 | 49.4% | 2.5% | G014690 | 59.2% | 12.9% | G014735 | 63.2% | 2.2% |
| 3 | G014646 | 43.1% | 0.1% | G014691 | 63.6% | 0.6% | G014736 | 57.0% | 3.7% |
| 4 | G014647 | 41.0% | 1.0% | G014692 | 62.7% | 2.4% | G014737 | 61.1% | 1.6% |
| 5 | G014648 | 36.7% | 0.8% | G014693 | 60.6% | 2.5% | G014738 | 61.9% | 5.7% |
| 6 | G014649 | 44.9% | 0.8% | G014694 | 42.7% | 0.8% | G014739 | 61.7% | 1.9% |
| 7 | G014650 | 24.7% | 1.2% | G014695 | 61.1% | 2.5% | G014740 | 63.9% | 0.4% |
| 8 | G014651 | 25.9% | 2.3% | G014696 | 52.7% | 2.7% | G014741 | 47.9% | 0.4% |
| 9 | G014652 | 2.6% | 0.3% | G014697 | 33.4% | 3.0% | G014742 | 1.0% | 0.4% |
| 10 | G014653 | 12.0% | 0.6% | G014698 | 56.6% | 2.2% | G014743 | 39.4% | 2.4% |
| 11 | G014654 | 37.2% | 2.5% | G014699 | 59.1% | 3.0% | G014744 | 67.0% | 4.0% |
| 12 | G014655 | 34.4% | 2.8% | G014700 | 60.7% | 2.2% | G014745 | 22.1% | 5.2% |
| 13 | G014656 | 46.9% | 1.8% | G014701 | 52.3% | 14.2% | G014746 | 59.9% | 0.1% |
| 14 | G014657 | 48.7% | 1.9% | G014702 | 60.1% | 5.7% | G014747 | 56.0% | 10.7% |
| 15 | G014658 | 47.9% | 4.9% | G014703 | 62.9% | 2.0% | G014748 | 54.8% | 3.4% |
| 16 | G014659 | 43.4% | 3.0% | G014704 | 63.9% | 3.0% | G014749 | 59.7% | 0.4% |
| 17 | G014660 | 49.8% | 2.8% | G014705 | 68.4% | 2.1% | G014750 | 51.1% | 1.6% |
| 18 | G014661 | 44.1% | 4.1% | G014706 | 58.8% | 7.8% | G014751 | 55.0% | 4.5% |
| 19 | G014662 | 50.3% | 0.4% | G014707 | 68.3% | 2.2% | G014752 | 48.8% | 5.4% |
| 20 | G014663 | 45.0% | 1.2% | G014708 | 65.3% | 2.4% | G014753 | 62.8% | 5.9% |
| 21 | G014664 | 38.4% | 5.2% | G014709 | 54.7% | 0.6% | G014754 | 59.9% | 0.1% |
| 22 | G014665 | 34.6% | 1.2% | G014710 | 55.9% | 6.4% | G014755 | 36.8% | 1.8% |
| 23 | G014666 | 36.0% | 0.6% | G014711 | 57.5% | 2.8% | G014756 | 55.0% | 2.0% |
| 24 | G014667 | 44.8% | 1.7% | G014712 | 56.1% | 10.4% | G014757 | 55.8% | 4.1% |
| 25 | G014668 | 44.7% | 1.1% | G014713 | 61.2% | 0.8% | G014758 | 57.9% | 6.1% |
| 26 | G014669 | 44.7% | 2.2% | G014714 | 59.5% | 5.4% | G014759 | 51.3% | 11.9% |
| 27 | G014670 | 48.3% | 6.2% | G014715 | 60.2% | 1.6% | G014760 | 57.4% | 0.4% |
| 28 | G014671 | 50.4% | 1.3% | G014716 | 66.0% | 9.4% | G014761 | 7.0% | 0.1% |
| 29 | G014672 | 56.9% | 4.5% | G014717 | 68.9% | 4.9% | G014762 | 10.0% | 1.6% |
| 30 | G014673 | 51.0% | 1.1% | G014718 | 68.3% | 0.7% | G014763 | 12.5% | 0.0% |
| 31 | G014674 | 52.1% | 0.6% | G014719 | 68.6% | 0.1% | G014764 | 13.4% | 2.1% |
| 32 | G014675 | 51.4% | 2.7% | G014720 | 67.3% | 3.1% | G014765 | 36.2% | 3.2% |
| 33 | G014676 | 53.3% | 3.6% | G014721 | 69.2% | 1.9% | G014766 | 46.4% | 5.7% |
| 34 | G014677 | 52.2% | 3.9% | G014722 | 68.4% | 4.6% | G014767 | 37.9% | 4.5% |
| 35 | G014678 | 57.6% | 4.2% | G014723 | 64.3% | 4.1% | G014768 | 37.3% | 8.8% |
| 36 | G014679 | 53.2% | 4.6% | G014724 | 61.2% | 4.4% | G014769 | 39.6% | 0.6% |
| 37 | G014680 | 50.1% | 5.6% | G014725 | 63.6% | 0.8% | G014770 | 27.4% | 0.1% |
| 38 | G014681 | 40.4% | 2.5% | G014726 | 58.0% | 4.2% | G014771 | 60.0% | 0.5% |
| 39 | G014682 | 48.5% | 3.7% | G014727 | 60.0% | 3.1% | G014772 | 63.5% | 4.7% |
| 40 | G014683 | 48.3% | 6.6% | G014728 | 63.3% | 0.9% | G014773 | 57.9% | 0.6% |
| 41 | G014684 | 51.6% | 0.4% | G014729 | 65.3% | 1.8% | G014774 | 54.3% | 4.7% |
| 42 | G014685 | 21.6% | 3.4% | G014730 | 65.8% | 0.4% | G014775 | 49.8% | 2.4% |
| 43 | G014686 | 32.3% | 0.6% | G014731 | 64.0% | 5.1% | G014776 | 2.3% | 0.6% |
| 44 | G014687 | 0.2% | 0.0% | G014732 | 0.4% | 0.1% | G014777 | 0.1% | 0.0% |
| 45 | G014688 | 18.6% | 0.6% | G014733 | 45.2% | 4.8% | G014778 | 11.3% | 1.5% |
| 46 | G000502 | 46.5% | 0.4% | G000502 | 46.5% | 0.4% | G000502 | 44.9% | 12.2% |
| 47 | G010039 | 29.4% | 1.1% | G010039 | 29.4% | 1.1% | G010039 | 20.5% | 1.2% |
| 48 | G012401 | 51.8% | 5.8% | G012401 | 51.8% | 5.8% | G012401 | 59.8% | 1.6% |

TABLE 12
Average Editing % in PCH cells for short-sgRNA guides
PCH - short-sgRNA

| | G000502 | | | G000486 | | | G000415 | | |
|---|---|---|---|---|---|---|---|---|---|
| Row | Variants | % Edit | SD | Variants | % Edit | SD | Variants | % Edit | SD |
| 1 | G014644 | 76.6% | 2.2% | G014689 | 10.7% | 1.4% | G014734 | 53.2% | 0.4% |
| 2 | G014645 | 73.9% | 2.1% | G014690 | 20.4% | 2.8% | G014735 | 64.0% | 0.5% |
| 3 | G014646 | 73.0% | 1.9% | G014691 | 21.9% | 0.2% | G014736 | 50.5% | 2.0% |
| 4 | G014647 | 69.8% | 3.1% | G014692 | 20.1% | 2.3% | G014737 | 57.7% | 2.7% |
| 5 | G014648 | 71.1% | 1.5% | G014693 | 20.8% | 1.6% | G014738 | 58.0% | 2.0% |
| 6 | G014649 | 70.9% | 1.0% | G014694 | 13.2% | 0.2% | G014739 | 57.3% | 0.7% |
| 7 | G014650 | 69.3% | 3.2% | G014695 | 26.7% | 0.3% | G014740 | 56.3% | 7.8% |
| 8 | G014651 | 67.4% | 0.3% | G014696 | 10.1% | 0.6% | G014741 | 41.4% | 2.0% |
| 9 | G014652 | 51.4% | 2.6% | G014697 | 2.5% | 0.1% | G014742 | 2.7% | 0.4% |
| 10 | G014653 | 60.2% | 0.6% | G014698 | 8.4% | 2.2% | G014743 | 45.4% | 2.3% |
| 11 | G014654 | 70.5% | 0.0% | G014699 | 22.0% | 3.2% | G014744 | 62.8% | 2.4% |
| 12 | G014655 | 72.5% | 0.1% | G014700 | 19.4% | 2.8% | G014745 | 38.9% | 2.8% |
| 13 | G014656 | 72.3% | 0.0% | G014701 | 29.7% | 4.7% | G014746 | 59.6% | 5.2% |
| 14 | G014657 | 71.4% | 0.8% | G014702 | 27.8% | 3.5% | G014747 | 47.5% | 3.5% |
| 15 | G014658 | 70.9% | 3.5% | G014703 | 25.5% | 3.8% | G014748 | 46.9% | 4.4% |
| 16 | G014659 | 69.6% | 1.2% | G014704 | 27.0% | 1.6% | G014749 | 54.6% | 3.3% |
| 17 | G014660 | 68.2% | 1.1% | G014705 | 25.0% | 0.8% | G014750 | 44.3% | 1.5% |
| 18 | G014661 | 64.7% | 0.1% | G014706 | 23.0% | 0.7% | G014751 | 51.3% | 2.2% |
| 19 | G014662 | 67.7% | 2.8% | G014707 | 25.4% | 1.2% | G014752 | 43.0% | 0.8% |
| 20 | G014663 | 69.3% | 1.8% | G014708 | 26.2% | 1.3% | G014753 | 47.6% | 3.9% |
| 21 | G04664 | 67.7% | 0.3% | G014709 | 25.7% | 5.9% | G014754 | 52.5% | 3.5% |
| 22 | G014665 | 66.5% | 0.1% | G014710 | 20.6% | 0.8% | G014755 | 35.8% | 0.9% |
| 23 | G014666 | 66.5% | 5.7% | G014711 | 22.6% | 0.9% | G014756 | 52.4% | 3.6% |
| 24 | G014667 | 68.7% | 4.8% | G014712 | 27.3% | 1.9% | G014757 | 55.6% | 0.8% |
| 25 | G014668 | 66.7% | 0.5% | G014713 | 27.9% | 2.0% | G014758 | 51.3% | 4.2% |
| 26 | G014669 | 65.9% | 1.2% | G014714 | 24.1% | 4.2% | G014759 | 50.8% | 1.5% |
| 27 | G014670 | 64.9% | 2.5% | G014715 | 24.3% | 2.2% | G014760 | 49.6% | 1.1% |
| 28 | G014671 | 68.6% | 3.0% | G014716 | 21.4% | 5.0% | G014761 | 9.6% | 1.0% |
| 29 | G014672 | 66.3% | 2.1% | G014717 | 24.5% | 2.1% | G014762 | 10.4% | 1.8% |
| 30 | G014673 | 65.3% | 1.6% | G014718 | 21.7% | 2.7% | G014763 | 13.5% | 0.5% |
| 31 | G014674 | 67.8% | 9.9% | G014719 | 20.1% | 1.7% | G014764 | 13.9% | 1.4% |
| 32 | G014675 | 63.3% | 0.0% | G014720 | 31.6% | 6.9% | G014765 | 35.0% | 3.1% |
| 33 | G014676 | 67.6% | 0.5% | G014721 | 24.8% | 2.8% | G014766 | 36.5% | 1.8% |
| 34 | G014677 | 66.3% | 3.4% | G014722 | 31.9% | 0.8% | G014767 | 36.7% | 0.9% |
| 35 | G014678 | 73.3% | 1.0% | G014723 | 33.8% | 5.5% | G014768 | 38.8% | 0.8% |
| 36 | G014679 | 64.1% | 2.3% | G014724 | 25.9% | 1.6% | G014769 | 31.7% | 1.0% |
| 37 | G014680 | 66.0% | 0.8% | G014725 | 25.8% | 1.4% | G014770 | 23.3% | 2.1% |
| 38 | G014681 | 65.9% | 1.0% | G014726 | 22.4% | 1.5% | G014771 | 50.9% | 0.0% |
| 39 | G014682 | 72.4% | 1.9% | G014727 | 28.0% | 1.8% | G014772 | 56.6% | 0.4% |
| 40 | G014683 | 69.2% | 1.6% | G014728 | 24.3% | 0.9% | G014773 | 52.9% | 1.6% |
| 41 | G014684 | 66.5% | 0.8% | G014729 | 27.3% | 0.9% | G014774 | 47.8% | 1.5% |
| 42 | G014685 | 48.5% | 5.3% | G014730 | 27.4% | 0.9% | G014775 | 41.7% | 4.9% |
| 43 | G014686 | 62.8% | 1.6% | G014731 | 12.5% | 3.4% | G014776 | 3.4% | 0.6% |
| 44 | G014687 | 0.5% | 0.3% | G014732 | 0.6% | 0.0% | G014777 | 0.3% | 0.1% |
| 45 | G014688 | 64.0% | 0.8% | G014733 | 6.5% | 1.1% | G014778 | 19.8% | 1.3% |
| 46 | G000502 | 67.2% | 0.5% | G000502 | 67.2% | 0.5% | G000502 | 63.6% | 2.3% |
| 47 | G010039 | 53.0% | 0.4% | G010039 | 53.0% | 0.4% | G010039 | 49.8% | 4.6% |
| 48 | G012401 | 68.3% | 3.5% | G012401 | 68.3% | 3.5% | G012401 | 73.2% | 3.4% |

### Example 5—In Vitro Editing in Primary Human Hepatocytes (PHH), Primary Cynomolgus Hepatocytes (PCH), and HepG2

sgRNAs targeting the human TTR gene were designed as shown in Table 1 and transfected into primary cyno hepatocytes (PCH), primary human hepatocytes (PHH), and HepG2 cells in vitro at concentrations as indicated in the figures and editing efficiency (e.g., percent editing) was measured by NGS, as described in Example 1. The LNPs used in these transfections were made according to LNP Procedure C in Example 1(F).

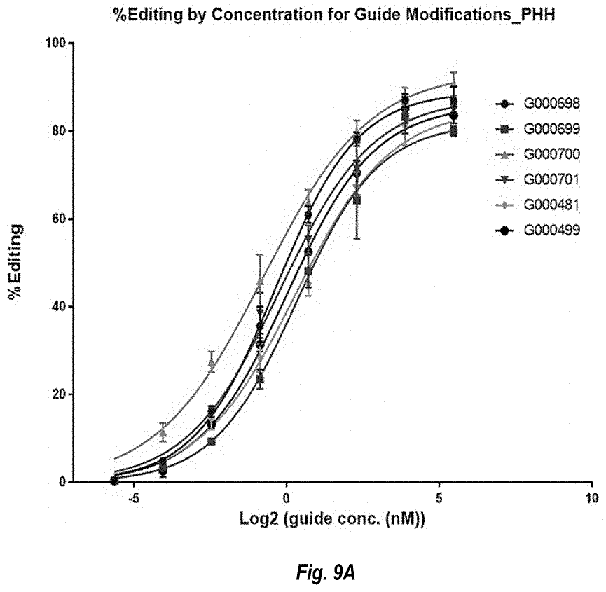

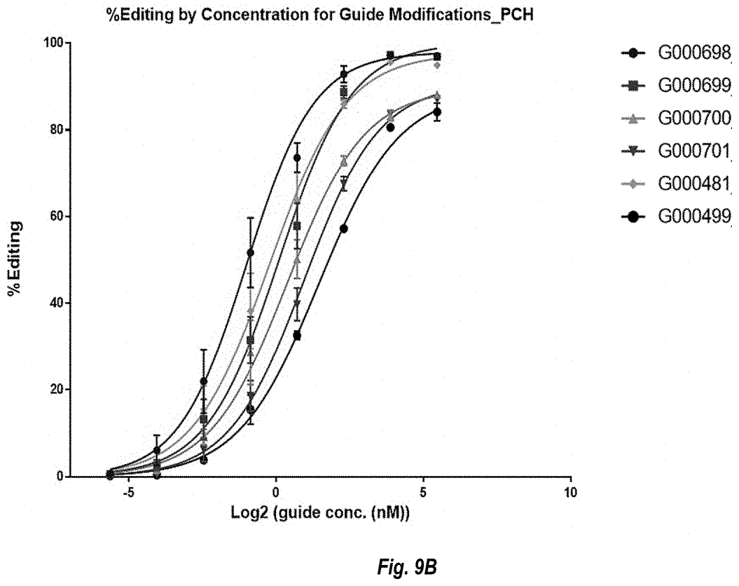

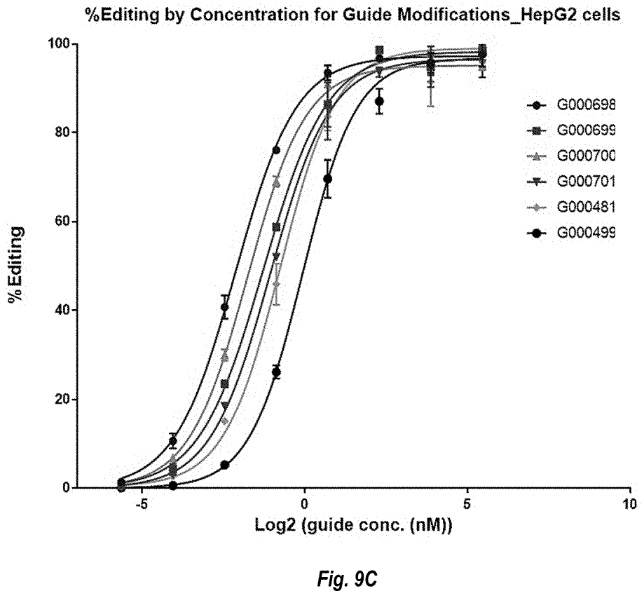

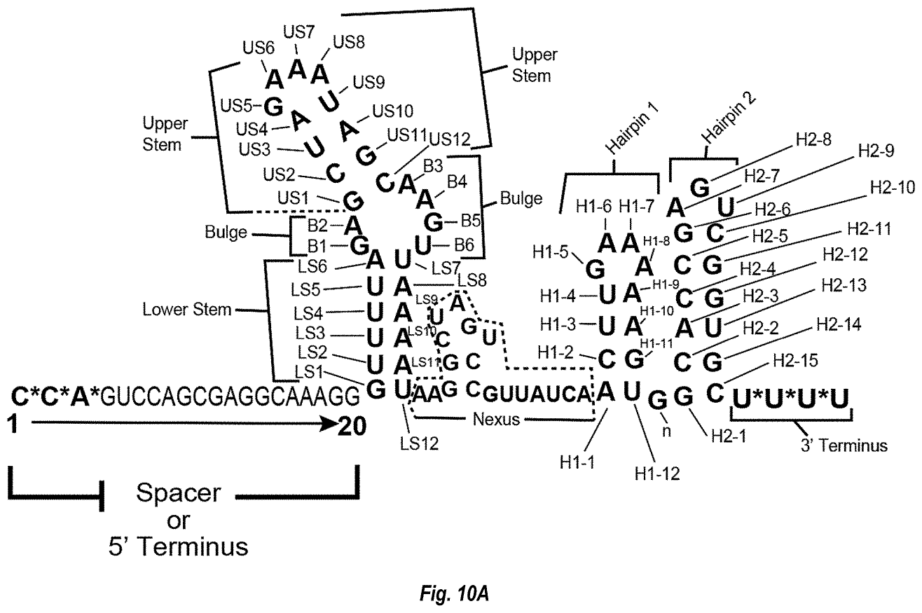

Dose response curves of editing efficiency by concentration are shown in FIGS. 9A (PHH), 9B (PCH), and 9C (HepG2). Tables 13A (PHH), 13B (PCH), and 13C (HepG2) summarize the results of FIGS. 9A-9C.

TABLE 13A
(PHH)

| sgRNA | min | max | EC50 | EC50error |
|---|---|---|---|---|
| G000698 | 0.43 | 87 | 0.981624 | 0.224339 |
| G000699 | 0.37 | 83.33 | 1.275023 | 0.243757 |
| G000700 | 0.73 | 90.73 | 0.757331 | 0.270775 |
| G000701 | 0.2 | 84.93 | 1.000807 | 0.248197 |
| G000481 | 0.17 | 81.3 | 1.305533 | 0.28657 |
| G000499 | 0.4 | 85.17 | 1.15425 | 0.237993 |

TABLE 13B
(PCH)

| sgRNA | min | max | EC50 | EC50error |
|---|---|---|---|---|
| G000698 | 0.65 | 97.15 | 0.482751 | 0.085198 |
| G000699 | 0.55 | 97.25 | 1.295555 | 0.242115 |
| G000700 | 0.65 | 88.15 | 1.287722 | 0.279182 |
| G000701 | 0.2 | 87.05 | 1.619526 | 0.350191 |
| G000481 | 0.55 | 95.6 | 1.070419 | 0.241465 |
| G000499 | 0.25 | 84.15 | 3.242486 | 0.847014 |

TABLE 13C
(HepG2)

| sgRNA | min | max | EC50 |
|---|---|---|---|
| G000698 | 1.4 | 97.55 | 0.290574 |
| G000699 | 0.6 | 98.65 | 0.452265 |
| G000700 | 1 | 97.5 | 0.37081 |
| G000701 | 0.35 | 96.85 | 0.496062 |
| G000481 | 0.35 | 98.6 | 0.537527 |
| G000499 | 0.1 | 97.9 | 1.136589 |

### Example 6—Nuclease Susceptibility and Stability of sgRNAs

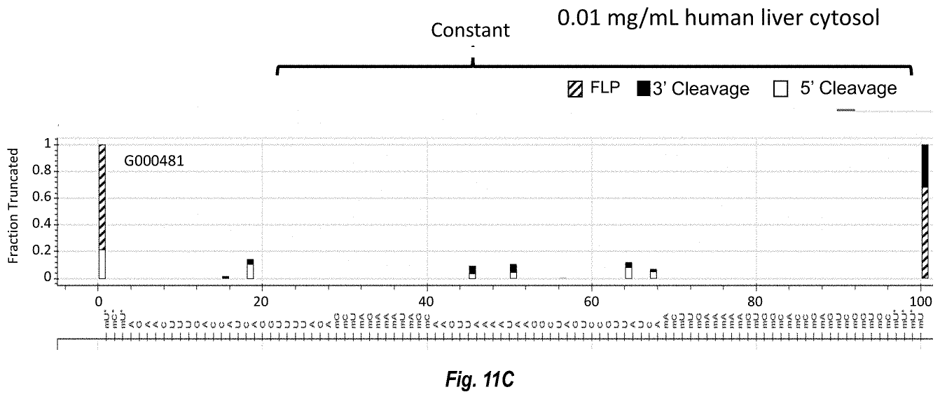

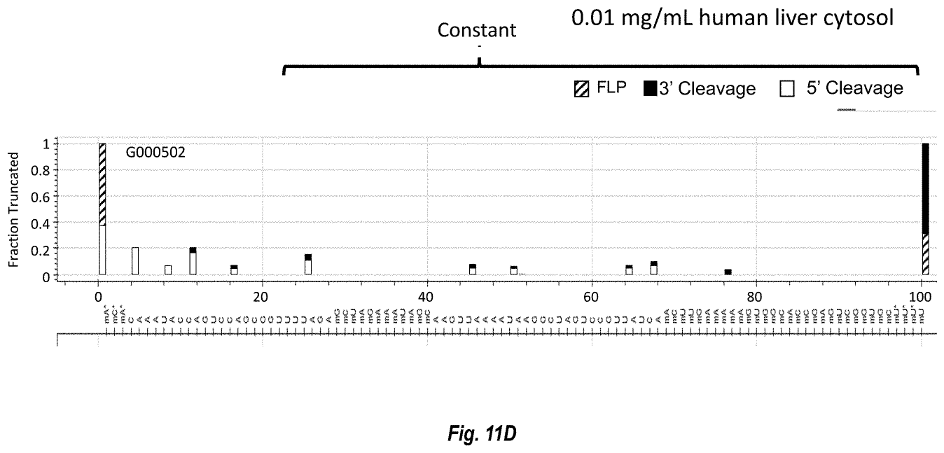

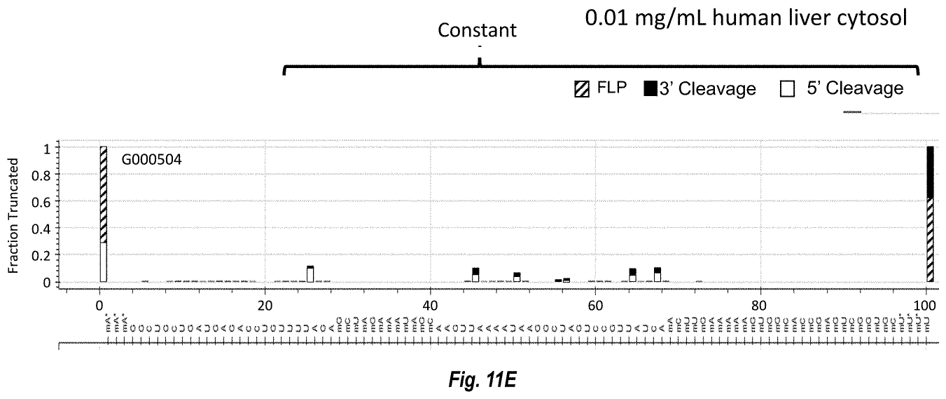

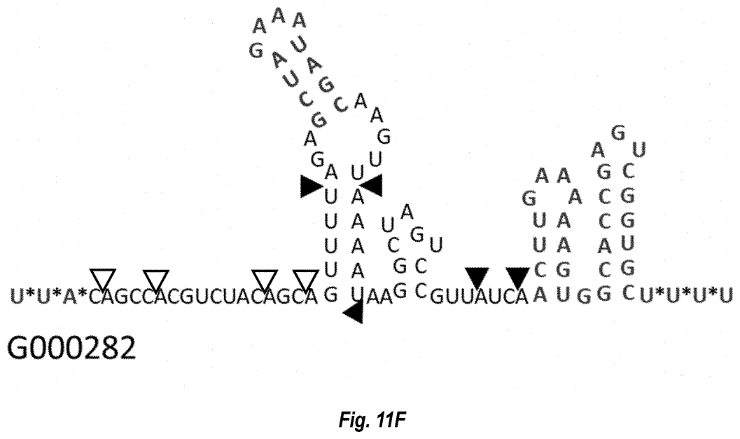

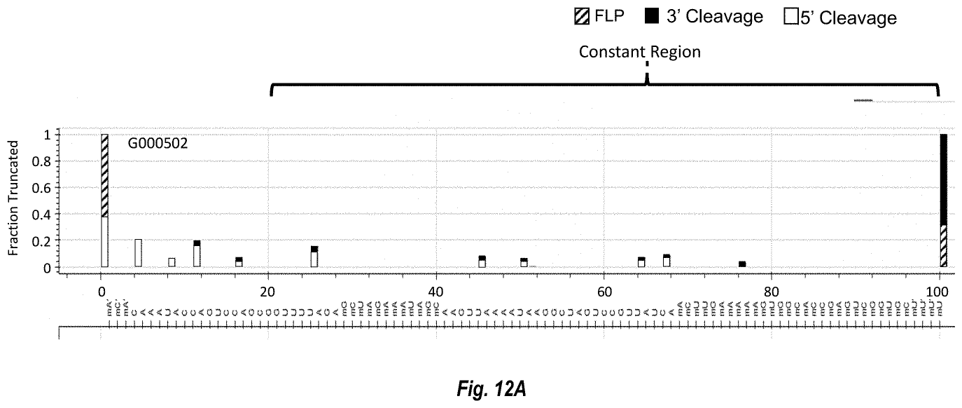

5′- and 3′-end-labeled versions of G282, G480, G481, G502, and G504 were assayed as described in Example 1(K). Fragment lengths were mapped onto the G282 sequence (FIG. 11A) and cleavage was noted predominantly at CpA and UpA (i.e., pyrimidine-adenine or “YA”) sites (illustrated in FIG. 10B), consistent with an RNase A-like endonucleolytic pattern. See Leu et. al., J. Biol Chem, 278:7300-09 (2003) (reporting RNase A cleavage at CpA and UpA sites). The cleavage sites observed for G282 are illustrated schematically on a possible secondary structure of this molecule in FIG. 10B.

Cleavage was consistently observed following nucleotides 25, 45, 50, 64, and 67 in G282, G480, G481, G502, and G504 (except that for G481, there was little to no cleavage at position 25) (FIG. 11A-D). All of these positions are YA sites (labeled YA sites 1, 5, 6, 7, 8, and 9 in FIG. 10B). Furthermore, in the spacer region, cleavage was observed in a generally YA-dependent manner, for example, following position 16 of G480; positions 15 and 18 of G481; and positions 4, 8, 11, and 16 of G502 (FIG. 11B-D). Notably, modifications at YA positions led to reduced cleavage (e.g., following positions 2, 31, 37, 40, and 83). YA positions in which at least the Y was a 2′-OMe nucleotide did not show significant cleavage, consistent with 2′-O-methylation protecting the adjacent 3′linkage from cleavage by RNAse A.

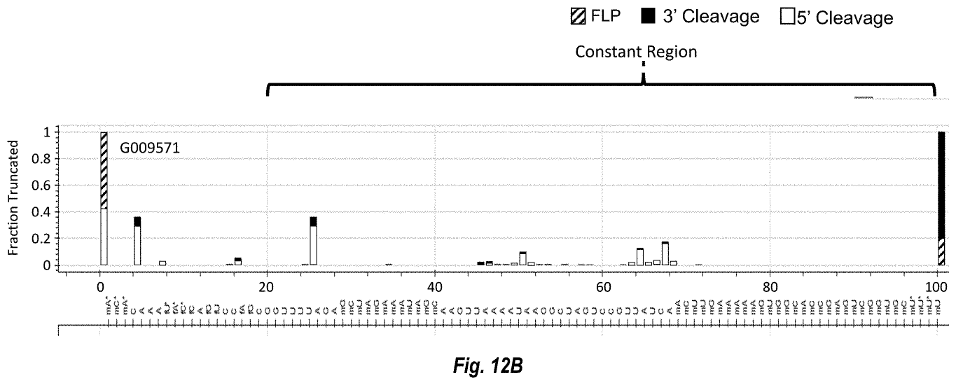

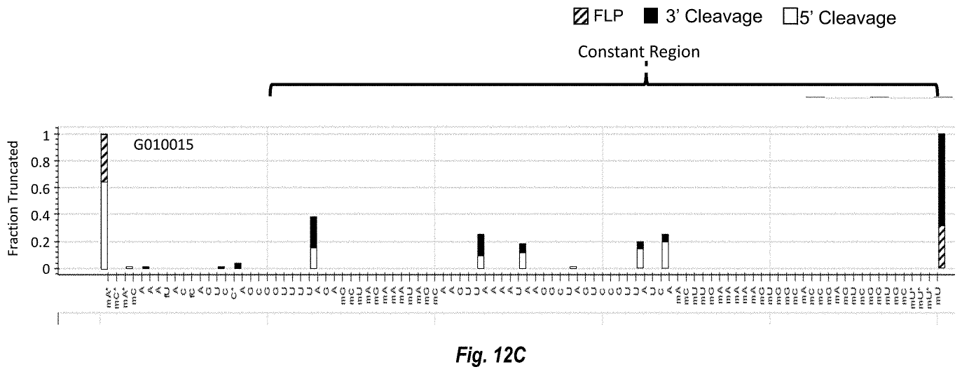

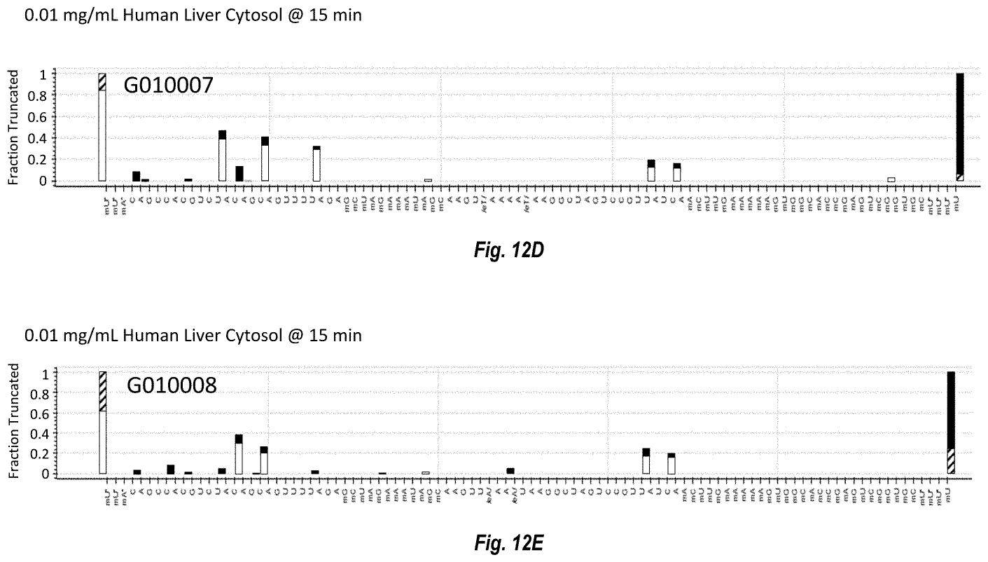

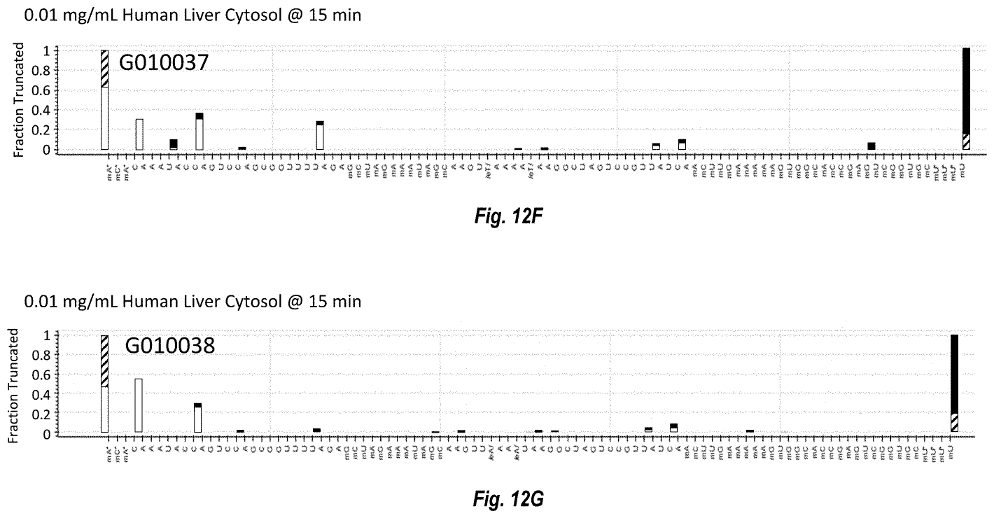

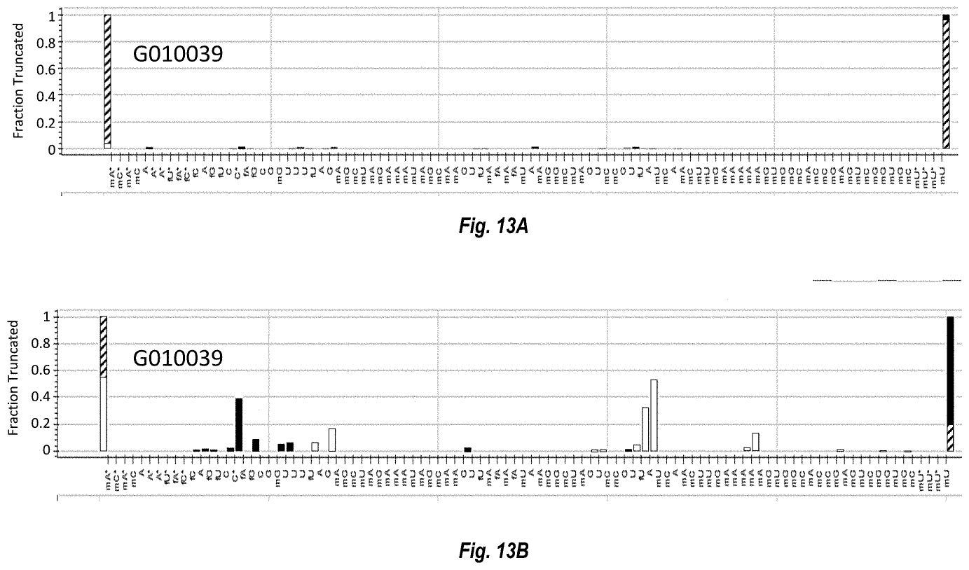

G502 was compared to sgRNAs with additional modifications in the guide domain (FIGS. 12A-C). In particular, G9571 includes 2′-fluoro modifications at certain positions including 8-11 and PS modifications at certain positions including 8-10 as shown in the Sequence Table. G10015 includes a 2′-OMe modification at position 4, 2′-fluoro modifications at positions 8 and 11, and a PS modification at position 16.

In G9571, cleavage after positions 8 and 11 was reduced or eliminated, consistent with nuclease protection by the 2′-fluoro modifications of these positions (FIG. 12B).

In G10015, cleavage after positions 4, 8, and 11 was reduced or eliminated, consistent with nuclease protection by the 2′-OMe modification of position 4 and the 2′-fluoro modifications of positions 8 and 11 (FIG. 12C). Cleavage after position 16 also occurred at a somewhat reduced level relative to G502, suggesting partial protection by the phosphorothioate modification at that position.

Assays on G282, G480, G481, G502, G504, and G509 assembled into ribonucleoproteins (RNPs) with Cas9 were also performed using a higher HLC concentration of 8.5 mg/ml but otherwise following the procedure described above. The RNPs showed reduced susceptibility compared to the experiments using sgRNA alone despite the higher HLC concentration, indicating that the sgRNA within an RNP is less accessible to nuclease, but the cleavage pattern remained qualitatively similar, with most cleavage occurring at YA sites (data not shown).

G10039, which comprises modifications at all YA sites, was assayed with 0.01 mg/ml HLC and found to show only a very small amount of cleavage at position 16, consistent with protection by the phosphorothioate modification at that position (FIG. 13A). Minimal amounts of cleavage were detectable at certain additional (non-YA) sites, but nearly all of the starting material remained intact throughout the incubation.

G10039 (as free sgRNA) was also treated with 8.5 mg/ml HLC. Degradation increased at position 16 and was also observed at several other positions, some of which were not YA sites (FIG. 13B).

### Example 7—Editing following Transfection with sgRNAs with Modifications at YA Sites

A series of sgRNAs were designed by systematically introducing 2′-OMe modifications at individual YA sites in the conserved regions that were unmodified in G282. Thus, the sgRNAs sequentially numbered from G9989-G9994 have 2′-OMe modifications at positions 25, 45, 50, 56, 64, and 67, respectively, which are positions LS5, LS7, LS12, N6, N14, and N17 as shown in FIG. 10A. These are the pyrimidines of YA sites 1, 5, 6, 7, 8, and 9 as shown in FIG. 10B. The sgRNAs sequentially numbered from G10019-G10024 have the same 2′-OMe modifications as G9989-G9994, respectively, but the nucleobase sequence is identical to that of G502 instead of G282.

Similarly, a series of sgRNAs were designed by systematically introducing 2′-fluoro modifications at individual YA sites in the conserved regions that were unmodified in G282. Thus, the sgRNAs sequentially numbered from G9995-G10000 have 2′-fluoro modifications at positions 25, 45, 50, 56, 64, and 67, respectively. The sgRNAs sequentially numbered from G10025-G10030 have the same 2′-fluoro modifications as G9995-G10000, respectively, but the nucleobase sequence is identical to that of G502 instead of G282.

A further series of sgRNAs were designed by systematically introducing phosphorothioate modifications at individual YA sites in the conserved regions that were unmodified in G282. Thus, the sgRNAs sequentially numbered from G10001-G10006 have 2′-fluoro modifications at positions 25, 45, 50, 56, 64, and 67. The sgRNAs sequentially numbered from G10031-G10036 have the same phosphorothioate modifications as G10001-G10006, respectively, but the nucleobase sequence is identical to that of G502 instead of G282.

Also tested were guides modified with ENA (G9878, G10007, and G10008 had the G282 sequence, and G10037 and G10038 had the G502 sequence). The modifications in G10007 and G10037 were at the 45th and 50th nucleotides (positions LS7 and LS12 as indicated in FIG. 10A). The ENA in G9878 was at the three 5′-terminal nucleotides and the fourth to second nucleotides from the 3′ end. The ENA in G10008 and G10038 was at the 46th and 49th nucleotides (positions LS8 and LS11 as indicated in FIG. 10A).

Also tested were guides modified with deoxyribonucleotides (G9423-G9427) and UNA (G9879), all of which had the G282 sequence. The positions of the modifications in these guides are shown in the Sequence Table.

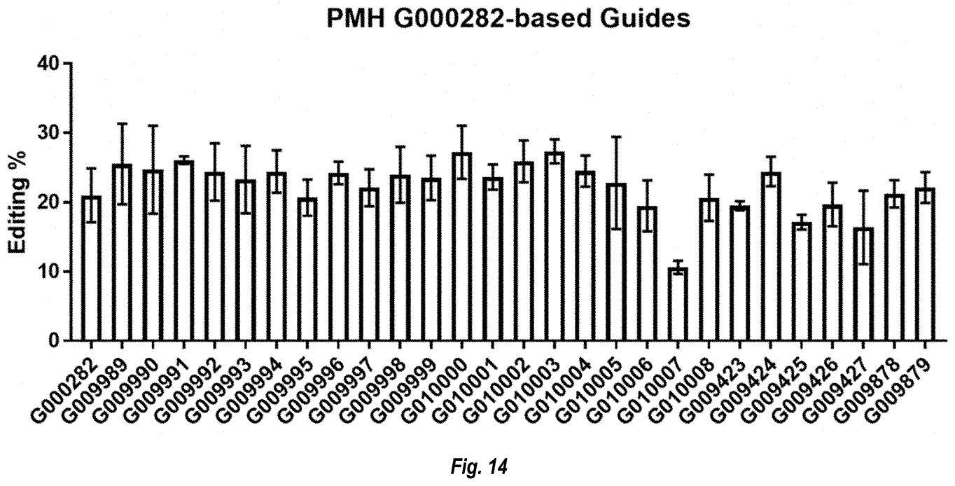

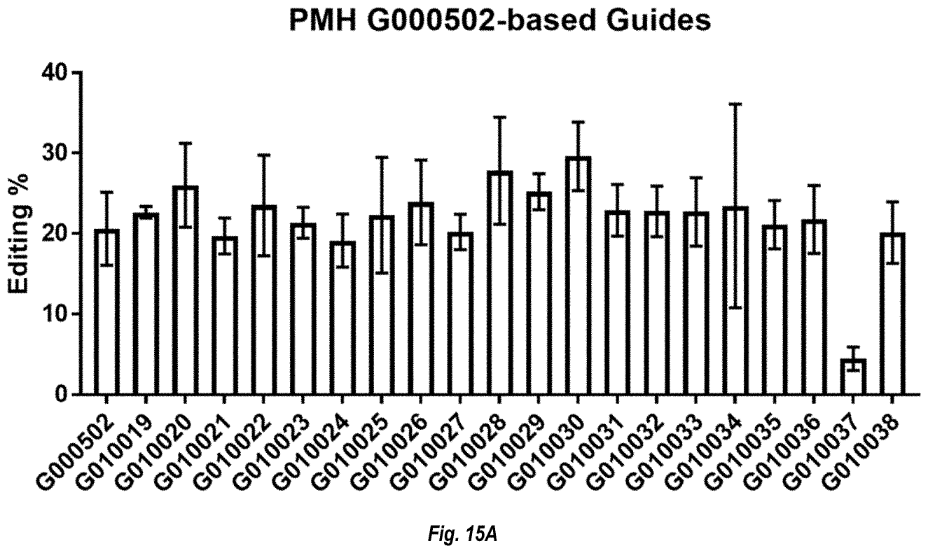

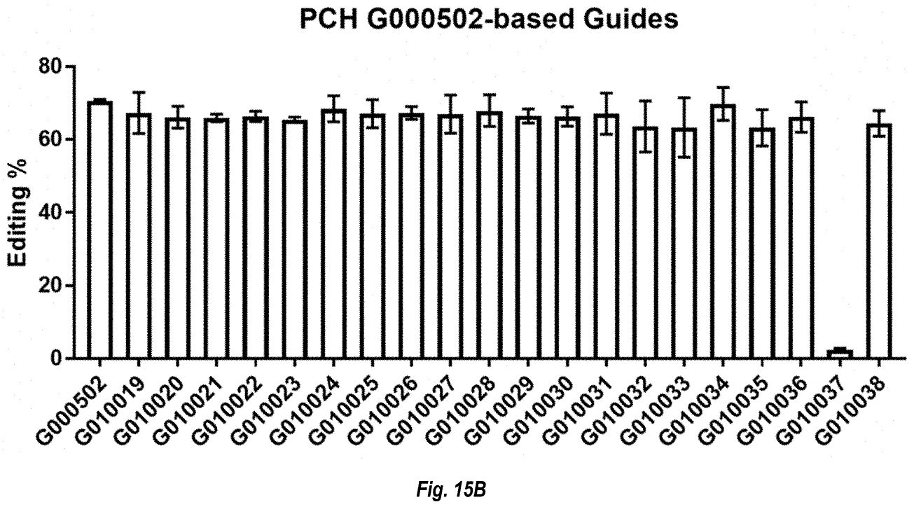

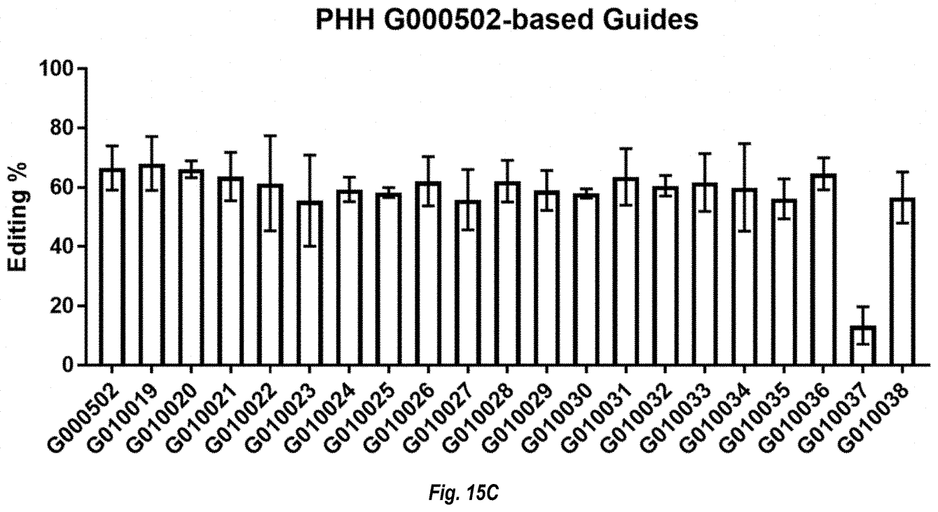

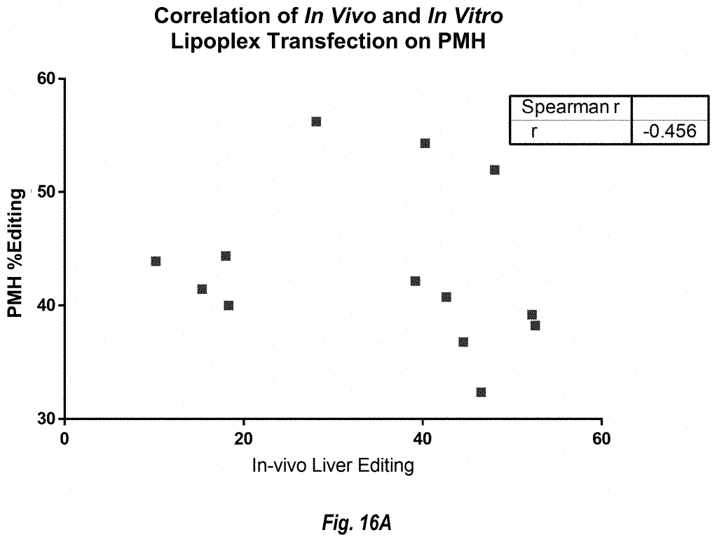

The guides described above were incorporated into lipoplexes and transfected into PMH as described above and percent editing was determined (FIG. 14 for guides matching the sequence of G282 and FIG. 15A for guides matching the sequence of G502). The guides with the sequence of G502 were also transfected into PCH and PHH as described above and percent editing was determined (FIG. 15B for PCH and FIG. 15C for PHH). The baseline reference (corresponding to the level of editing indicated by the dotted line) was G282 in FIGS. 14 and G502 in FIG. 15A. All of the 2′-OMe, 2′-fluoro, and phosphorothioate modifications introduced in G9989-G10006 and G10019-G10036 were tolerated in that editing activity did not substantially decrease. Additionally, the other modifications were generally tolerated as well. The ENA modification of positions 45-50 in G10007 and G10037 showed lower percent editing activity.

### Example 8—Primary Cell Editing following Transfection with sgRNAs

Several series of modified sgRNAs were designed based on the nucleobase sequences of G000282 and G000502. Specific modifications are described in Table 1. G00282 variants were assayed for editing in duplicate (unless otherwise noted) in primary mouse hepatocyte cells (PMH) in vitro. G000502 variants were similarly assayed in duplicate (unless otherwise noted) in primary cyno hepatocyte (PCH) and PMH cells in vitro. All data is reported in Tables 14 and 15 below.

A series of sgRNAs were designed assaying modifications in the guide domain combined with the modified conserved region described in SEQ ID No. 201. The sgRNAs sequentially numbered from G012421-G012425, G012689, G012690, G012426-G012431 have the same modifications as G012693-G012705, respectively, but the nucleobase sequence is identical to that of G000502 instead of 0000282. Data for these guides are presented in Table 14.

Similarly, a series of sgRNAs were designed assaying modifications in the conserved region combined with the modified guide domain of either the G0000502 variant, 6012402, or the modified guide domain of G000282 variant, G009533. The sgRNAs sequentially numbered from G012432-G012438, G012691, G012439-G012440, G012692 have the same modifications as G012706-G12716, respectively, but the nucleobase sequence is identical to that of G000502 instead of G000282. Data for these guides are presented in Table 14.

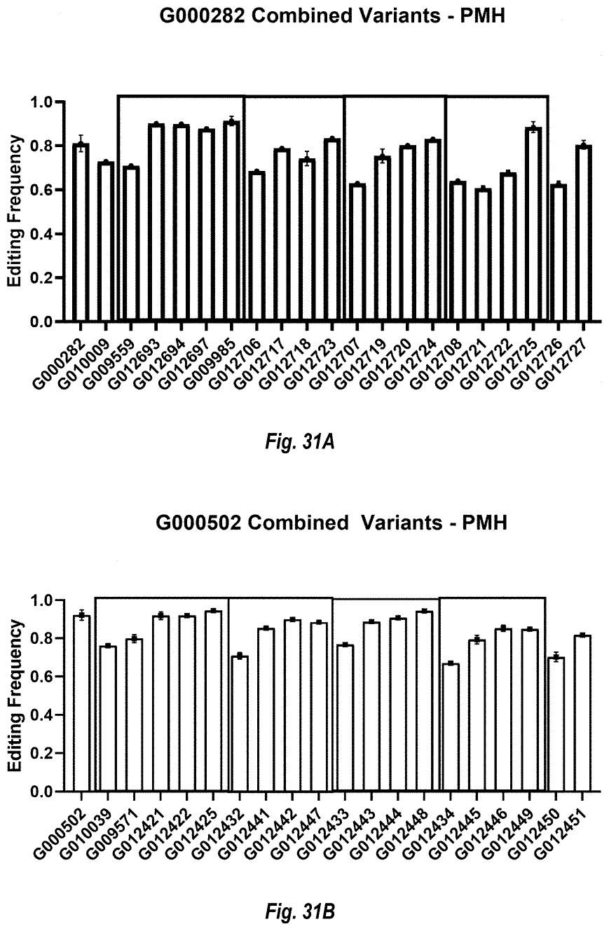

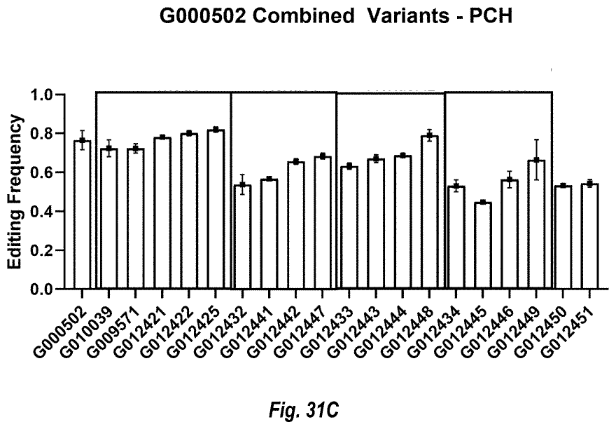

A further series of sgRNAs were designed combining various guide domain and conserved region modification patterns. The sgRNAs sequentially numbered from G012441-G012451 sequentially have the same modifications as G012717-G012727, respectively, but the nucleobase sequence is identical to that of G000502 instead of G000282. Data for these guides are presented in Table 14 and FIG. 31A-C.

Similarly, a series of sgRNAs were designed assaying modifications in the context of short single guide variants of G000282 and G000502. The sgRNAs sequentially numbered from G012452-G012461 are based on the short guide variant of G000502, specifically G012401. These modified variants have the same modifications as G012728-G12737, respectively, but the nucleobase sequence of G012728-G12737 is identical to that of G000639, the short guide variant of G000282. Data for this series of guides are presented in Table 14.

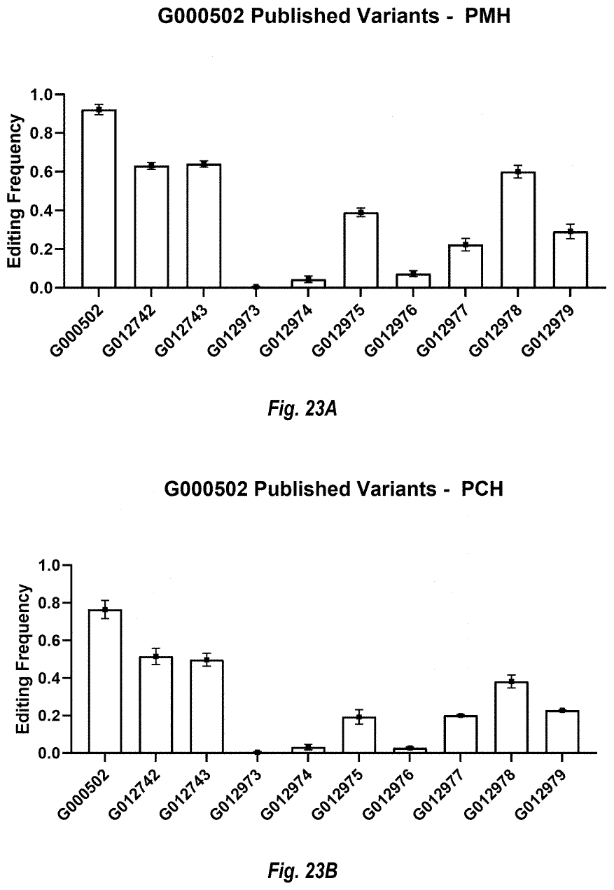

Lastly, a series of sgRNAs as indicated in Table 15 were designed assaying modifications in the nucleobase sequence of G000502 (See Table 1 for sgRNA nucleotide sequences). Data for this series of guides are presented in Table 15 and FIG. 23A-B.

TABLE 14
Primary cell editing with modified guides

| | PMH | | | PCH | | PMH | |
|---|---|---|---|---|---|---|---|
| G000282 | % | | G000502 | % | | % | |
| Variants | Editing | SD | Variants | Editing | SD | Editing | SD |
| G000282 | 81.0% | 3.7% | G000502 | 76.5% | 4.9% | 92.3% | 2.6% |
| G000639 | 83.4% | 1.6% | G012401 | 71.7% | 3.3% | 91.1% | 0.0% |
| G009559 | 70.9% | 0.4% | G009571 | 72.3% | 2.4% | 79.9% | 2.1% |
| G009985 | 91.3% | 2.1% | ND | ND | ND | ND | ND |
| G010009 | 72.9% | 0.5% | G010039 | 72.4% | 4.3% | 76.1% | 1.0% |
| G012693 | 90.2% | 0.4% | G012421 | 78.1% | 1.0% | 92.0% | 2.0% |
| G012694 | 89.9% | 0.4% | G012422 | 80.1% | 1.3% | 92.0% | 0.8% |
| G012695 | 88.7% | 0.7% | G012423 | 76.4% | 4.7% | 92.6% | 0.4% |
| G012696 | 85.7% | 0.8% | G012424 | 79.1% | 3.6% | 93.1% | 0.6% |
| G012697 | 87.8% | 0.7% | G012425 | 82.0% | 1.3% | 94.6% | 0.3% |
| G012698 | 68.7% | 0.8% | G012689 | 60.6% | 1.6% | 86.1% | 2.1% |
| G012699 | 68.1% | 1.8% | G012690 | 67.0% | 2.3% | 89.6% | 0.5% |
| G012700 | 73.9% | 0.1% | G012426 | 78.6% | 2.9% | 93.2% | 1.8% |
| G012701 | 75.7% | 1.6% | G012427 | 66.2% | 3.0% | 80.4% | 1.0% |
| G012702 | 69.4% | 0.1% | G012428 | 66.1% | 0.5% | 81.8% | 0.8% |
| G012703 | 86.3% | 2.6% | G012429 | 59.7% | ND | 79.3% | 1.7% |
| G012704 | 64.6% | 2.3% | G012430 | 65.6% | 0.8% | 75.8% | 1.8% |
| G012705 | 67.3% | 1.1% | G012431 | 64.1% | 0.6% | 76.3% | 2.5% |
| G012706 | 68.4% | 0.4% | G012432 | 53.8% | 5.1% | 70.8% | 1.7% |
| G012707 | 62.9% | 0.4% | G012433 | 63.2% | 1.6% | 76.7% | 0.1% |
| G012708 | 64.0% | 0.1% | G012434 | 53.1% | 3.0% | 67.0% | 0.4% |
| G012709 | 65.8% | 0.7% | G012435 | 62.2% | 0.4% | 70.7% | 0.4% |
| G012710 | 43.8% | 0.5% | G012436 | 47.3% | 3.3% | 64.9% | 2.8% |
| G012711 | 54.6% | 1.6% | G012437 | 51.2% | 4.7% | 67.7% | 0.2% |
| G012712 | 65.9% | 1.6% | G012438 | 60.0% | 1.3% | 68.7% | 2.1% |
| G012713 | 67.6% | 0.3% | G012691 | 61.5% | 9.0% | 74.4% | 0.5% |
| G012714 | 66.3% | 2.1% | G012439 | 39.4% | 1.8% | 58.0% | 4.3% |
| G012715 | 59.3% | 1.8% | G012440 | 40.4% | 3.2% | 65.5% | 3.3% |
| G012716 | 60.9% | 0.5% | G012692 | 43.8% | 1.5% | 63.1% | 0.1% |
| G012717 | 78.8% | 0.4% | G012441 | 56.8% | 0.3% | 85.3% | 0.8% |
| G012718 | 74.1% | 3.3% | G012442 | 65.6% | 1.5% | 90.0% | 0.3% |
| G012719 | 75.3% | 3.1% | G012443 | 67.0% | 2.0% | 88.8% | 0.6% |
| G012720 | 80.1% | 0.0% | G012444 | 68.7% | 1.2% | 90.9% | 0.1% |
| G012721 | 60.6% | 1.1% | G012445 | 44.9% | 1.1% | 79.3% | 2.3% |
| G012722 | 67.8% | 1.1% | G012446 | 56.4% | 4.2% | 85.2% | 1.6% |
| G012723 | 83.3% | 0.7% | G012447 | 68.4% | 1.5% | 88.5% | 0.6% |
| G012724 | 82.9% | 0.7% | G012448 | 79.0% | 3.0% | 94.5% | 0.5% |
| G012725 | 88.5% | 2.5% | G012449 | 66.4% | 10.3% | 84.7% | 0.4% |
| G012726 | 62.6% | 1.3% | G012450 | 53.3% | 0.9% | 70.2% | 2.5% |
| G012727 | 80.4% | 1.9% | G012451 | 54.4% | 2.0% | 81.6% | 0.7% |
| G012728 | 65.7% | 2.0% | G012452 | 65.2% | ND | 73.4% | 0.8% |
| G012729 | 65.9% | 3.7% | G012453 | 67.5% | 0.5% | 75.4% | 0.1% |
| G012730 | 63.4% | 2.7% | G012454 | 60.7% | 0.1% | 75.3% | 0.4% |
| G012731 | 67.4% | 3.8% | G012455 | 63.8% | 1.1% | 71.9% | 1.4% |
| G012732 | 69.7% | 0.0% | G012456 | 65.5% | 0.1% | 73.3% | 0.8% |
| G012733 | 70.1% | 2.9% | G012457 | 70.0% | 10.9% | 76.5% | 1.1% |
| G012734 | 65.2% | 1.4% | G012458 | 52.5% | 2.1% | 73.8% | 3.5% |
| G012735 | 59.9% | 0.7% | G012459 | 55.7% | 4.4% | 74.7% | 0.3% |
| G012736 | 55.0% | 0.8% | G012460 | 38.7% | 1.1% | 61.6% | 4.1% |
| G012737 | 61.7% | 2.8% | G012461 | 51.1% | 0.1% | 69.6% | 1.1% |
| G012741 | 70.7% | 3.6% | G012402 | 71.3% | 2.8% | 77.7% | 0.8% |

TABLE 15
Primary Cell editing with Modified guides

| | | |
|---|---|---|
| G000502 | PCH | PMH |

| Variants | % Editing | SD | % Editing | SD |
|---|---|---|---|---|
| G000502 | 76.5% | 4.9% | 92.3% | 2.6% |
| G012742 | 51.6% | 4.2% | 63.1% | 1.8% |
| G012743 | 49.9% | 3.5% | 64.1% | 1.6% |
| G012973 | 0.5% | 0.4% | 0.6% | 0.2% |
| G012974 | 3.3% | 1.5% | 4.4% | 1.7% |
| G012975 | 19.3% | 4.0% | 39.1% | 2.2% |
| G012976 | 2.8% | 0.5% | 7.3% | 1.5% |
| G012977 | 20.1% | 0.6% | 22.4% | 3.3% |
| G012978 | 38.3% | 3.5% | 60.1% | 3.3% |
| G012979 | 23.0% | 0.2% | 29.3% | 3.7% |

### Example 9—In Vitro Editing of Modified Guides Targeting HAO1 and SERPINA1

Lipid nanoparticle (LNP) formulations of modified sgRNAs targeting were tested on primary human hepatocytes and primary cynomolgus hepatocytes in a dose response assay with guides targeting the human genes HAO1 or LDHA. All methods are as described in Example 1 unless otherwise noted. Both cell lines were incubated at 37° C., 5% CO₂ for 48 hours prior to treatment with LNPs. LNPs were incubated in media containing 3% cynomolgus serum at 37° C. for 10 minutes and administered to cells in amounts as further provided herein. Post-incubation the LNPs were added to the human or cynomolgus hepatocytes in an 8 point 3-fold dose response curve starting at 300 ng Cas9 mRNA. The cells were lysed 96 hours post-treatment for NGS analysis as described in Example 1

Table 16 shows the average editing and standard deviation the tested control sgRNAs at 10.75 nM delivered with Spy Cas9 via LNP in PHH and PCH. These samples were generated in triplicate.

TABLE 16
Primary cell editing with modified guides
targeting HAO1 at 10.75 nM guide.

| Cell type | GUIDE | Edit | SD |
|---|---|---|---|
| PHH | G000480 | 0.990 | 0.002 |
| | G000502 | 0.909 | 0.022 |
| PCH | G000480 | 0.095 | 0.018 |
| | G000502 | 0.912 | 0.012 |

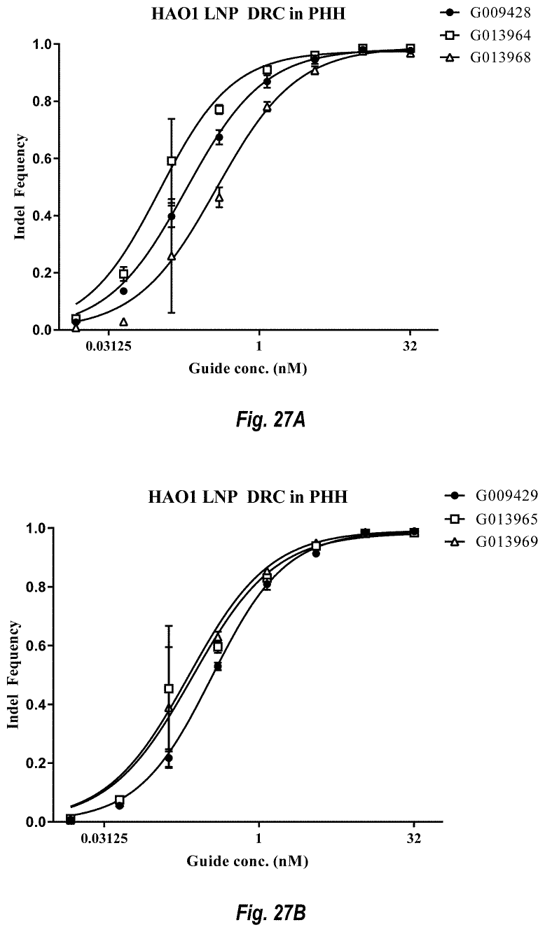

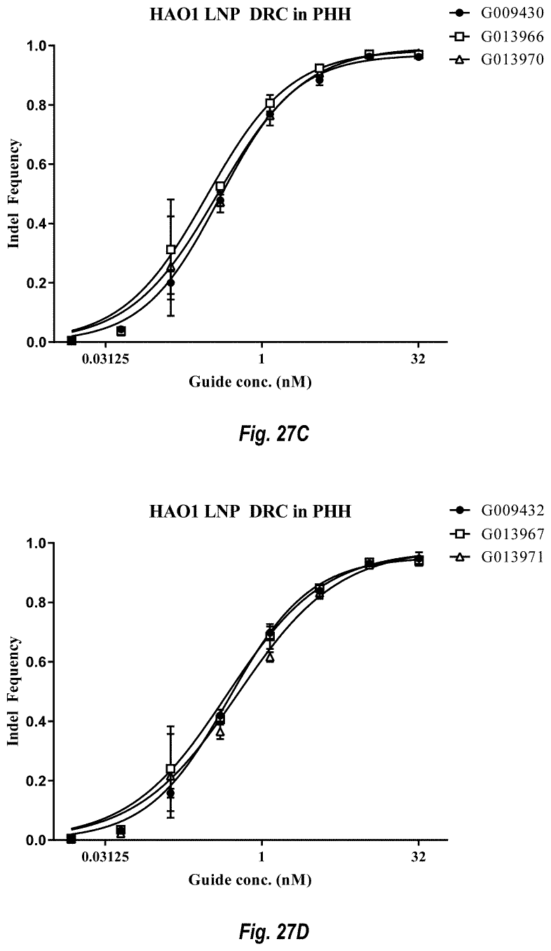

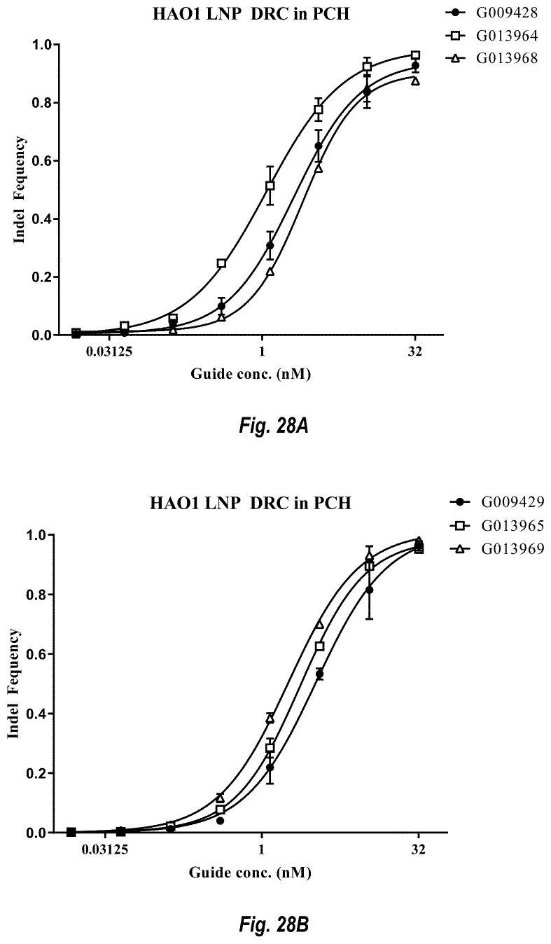

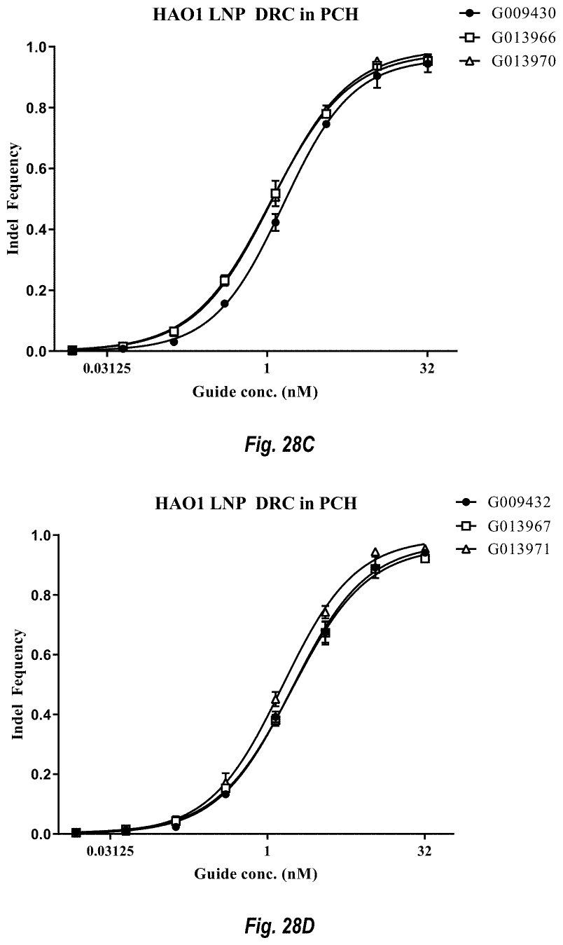

Table 17 shows the average editing and standard deviation for sgRNAs targing HAO1 delivered with Spy Cas9 via LNP to PHH or PCH. These samples were generated in at least duplicate. The dose response curve plot for these data are shown in FIGS. 27A-D and 28A-D.

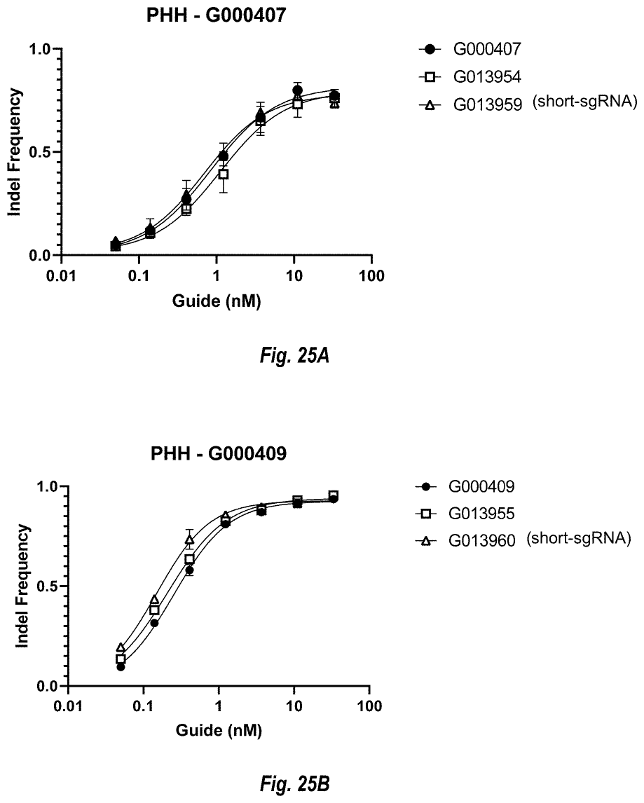

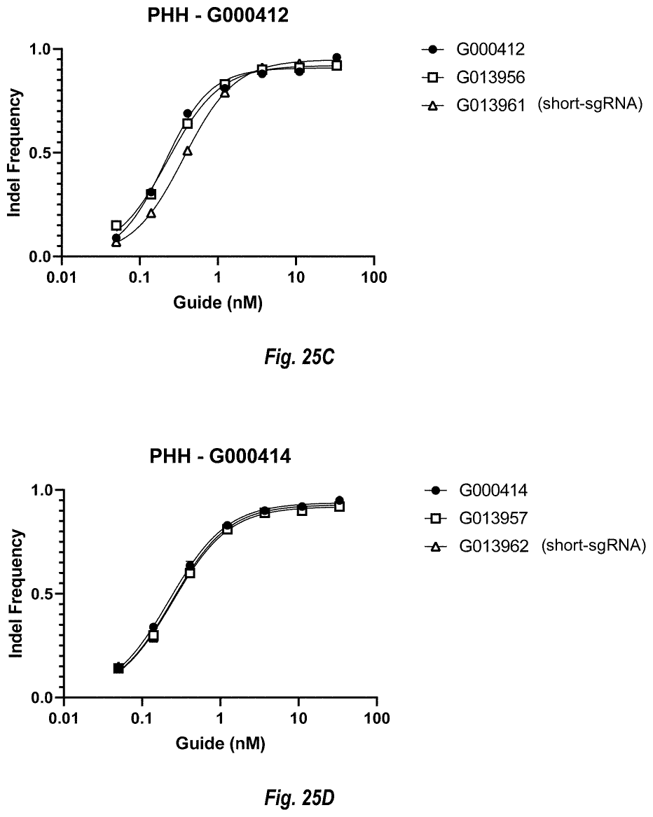

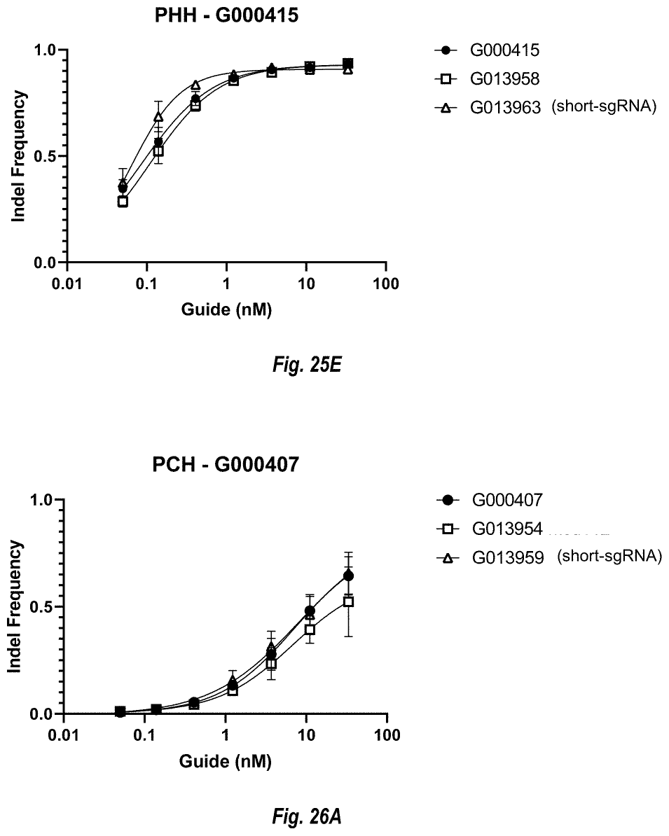

Table 18 shows the average editing and standard deviation for sgRNAs targeting SerpinA1 delivered with Spy Cas9 via LNP to PHH. G000480 and G000502 are controls that target TTR. These samples were generated in at least duplicate. The dose response curve plot for these data are shown in FIGS. 25A-E.

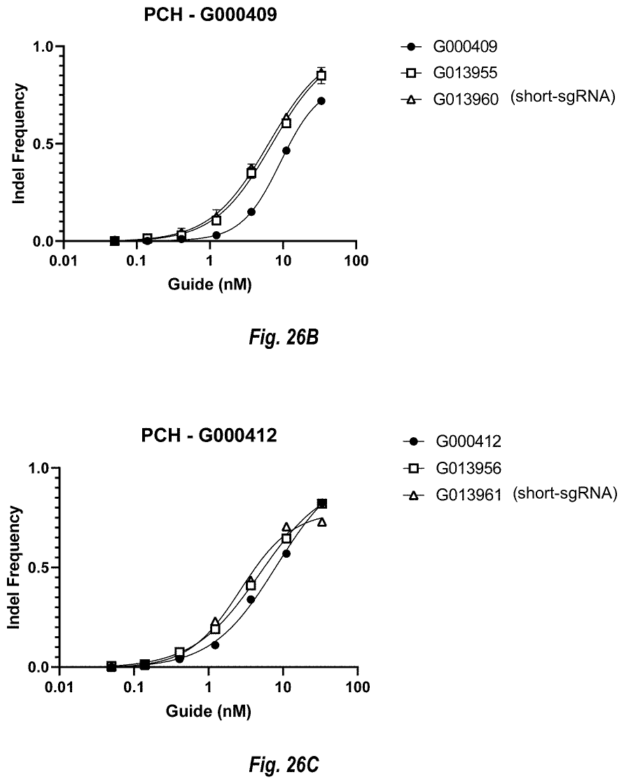

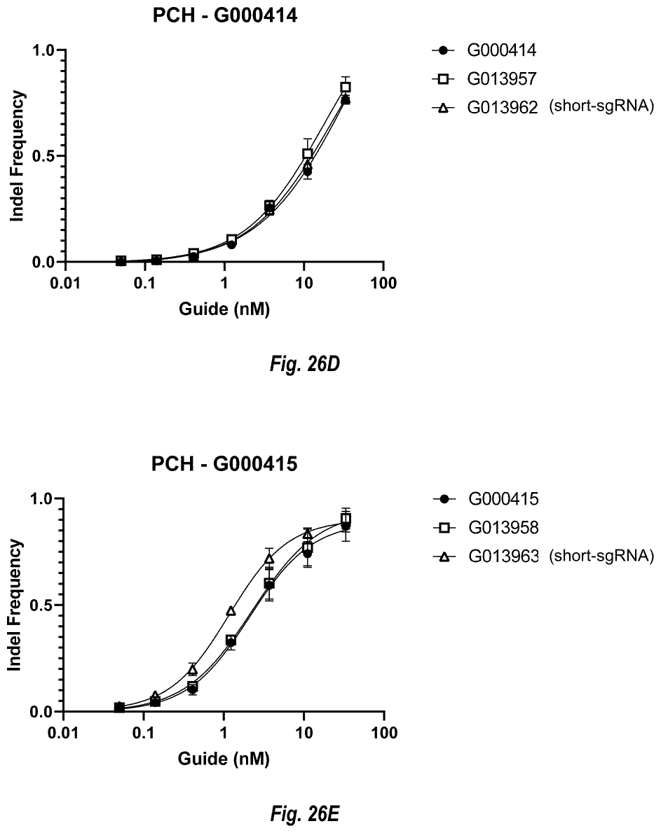

Table 19 shows the average editing and standard deviation for sgRNAs targeting SerpinA1 delivered with Spy Cas9 via LNP to PCH. G000480 and G000502 are controls that target TTR. These samples were generated in at least duplicate. The dose response curve plot for these data are shown in FIGS. 26A-E.

TABLE 17
Editing in primary cells with modified guides targeting HAO1

| Guide (nM) | | 32.260 | | 10.753 | | 3.584 | | 1.195 | | 0.398 | | 0.133 | | 0.044 | | 0.015 | |
|---|---|---|---|---|---|---|---|---|---|---|---|---|---|---|---|---|---|
| | Guide | % Edit | SD | Edit | SD | Edit | SD | Edit | SD | Edit | SD | Edit | SD | Edit | SD | Edit | SD |
| PHH | G009428 | 97.7 | 0.2 | 98.0 | 0.5 | 94.5 | 1.5 | 86.9 | 2.2 | 67.4 | 2.5 | 39.7 | 3.8 | 13.6 | 0.9 | 2.7 | 1.0 |
| | G013964 | 98.5 | 0.4 | 98.5 | 0.4 | 96.1 | 0.8 | 91.0 | 1.3 | 77.1 | 1.6 | 59.1 | 14.7 | 19.6 | 2.5 | 3.9 | 0.8 |
| | G013968 | 96.8 | 0.8 | 97.5 | 0.4 | 90.8 | 1.4 | 78.1 | 1.7 | 46.4 | 3.5 | 25.9 | 19.9 | 2.9 | 0.8 | 0.8 | 0.3 |
| | G009429 | 98.9 | 0.1 | 98.4 | 0.5 | 91.3 | 0.6 | 80.9 | 1.9 | 52.9 | 1.3 | 21.7 | 3.0 | 5.5 | 0.7 | 0.7 | 0.3 |
| | G013965 | 98.4 | 0.6 | 98.2 | 0.2 | 93.9 | 1.2 | 83.0 | 1.1 | 59.6 | 2.1 | 45.3 | 21.4 | 7.5 | 0.6 | 1.1 | 0.1 |
| | G013969 | 98.8 | 0.3 | 98.6 | 0.2 | 94.9 | 0.3 | 85.3 | 0.3 | 62.9 | 1.8 | 38.9 | 20.6 | 6.5 | 1.0 | 0.6 | 0.3 |
| | G009430 | 96.2 | 0.4 | 96.3 | 0.3 | 88.4 | 1.8 | 76.9 | 0.4 | 47.8 | 2.0 | 20.0 | 3.8 | 4.3 | 1.1 | 0.5 | 0.1 |
| | G013966 | 97.0 | 0.5 | 97.1 | 0.3 | 92.4 | 1.0 | 80.6 | 2.8 | 52.6 | 1.2 | 31.2 | 16.9 | 3.7 | 0.7 | 0.5 | 0.2 |
| | G013970 | 97.5 | 0.1 | 97.0 | 0.2 | 90.8 | 0.4 | 76.4 | 3.4 | 47.2 | 3.5 | 25.6 | 16.8 | 3.5 | 0.7 | 0.5 | 0.1 |
| | G009432 | 94.8 | 2.1 | 93.1 | 0.7 | 83.9 | 1.9 | 69.8 | 2.1 | 41.9 | 2.0 | 15.8 | 1.5 | 3.2 | 0.5 | 0.5 | 0.3 |
| | G013967 | 94.2 | 0.4 | 93.5 | 0.8 | 84.8 | 0.6 | 68.5 | 4.2 | 40.6 | 1.8 | 24.0 | 14.3 | 3.5 | 0.8 | 0.5 | 0.2 |
| | G013971 | 93.6 | 0.2 | 92.6 | 0.6 | 83.2 | 2.0 | 61.6 | 1.6 | 36.5 | 2.5 | 21.6 | 14.1 | 2.2 | 0.6 | 0.3 | 0.3 |
| PCH | G009428 | 92.8 | 2.4 | 83.6 | 5.5 | 65.1 | 5.5 | 30.8 | 4.8 | 9.9 | 2.9 | 3.7 | 1.5 | 0.7 | 0.2 | 0.4 | 0.2 |
| | G013964 | 96.3 | 0.5 | 92.4 | 3.1 | 77.6 | 3.9 | 51.4 | 6.6 | 24.7 | 0.0 | 5.8 | ND | 3.2 | 1.3 | 0.8 | 0.2 |
| | G013968 | 87.5 | 0.1 | 84.9 | 4.6 | 57.4 | 1.2 | 22.0 | ND | 6.2 | 0.3 | 1.8 | 0.8 | 1.4 | 0.7 | 0.4 | 0.3 |
| | G009429 | 96.3 | 1.6 | 81.5 | 9.8 | 53.3 | 1.9 | 21.9 | 5.5 | 4.0 | 0.6 | 1.2 | 0.5 | 0.3 | 0.2 | 0.2 | 0.1 |
| | G013965 | 95.2 | 0.6 | 89.5 | 1.1 | 62.6 | 0.5 | 28.4 | 3.2 | 7.7 | 0.9 | 2.1 | 0.3 | 0.3 | 0.2 | 0.2 | 0.1 |
| | G013969 | 98.1 | 0.7 | 93.1 | 3.1 | 70.0 | 0.5 | 38.5 | 1.6 | 11.6 | 1.4 | 2.6 | 0.2 | 0.7 | 0.3 | 0.3 | 0.1 |
| | G009430 | 94.4 | 2.8 | 90.4 | 3.9 | 74.6 | 1.0 | 42.3 | 2.8 | 15.6 | 0.4 | 3.0 | 0.9 | 0.8 | 0.1 | 0.2 | 0.1 |
| | G013966 | 95.3 | 0.7 | 93.8 | 1.0 | 77.9 | 0.8 | 51.8 | 4.2 | 23.2 | 1.7 | 6.5 | 1.0 | 1.5 | 0.5 | 0.3 | 0.2 |
| | G013970 | 95.9 | 1.6 | 95.3 | 0.2 | 79.3 | 1.4 | 50.6 | 0.3 | 23.4 | 0.7 | 6.2 | 1.0 | 1.4 | 0.2 | 0.2 | 0.1 |
| | G009432 | 94.1 | 0.8 | 89.2 | 3.6 | 67.5 | 3.5 | 39.2 | 1.8 | 13.3 | 1.2 | 2.3 | 0.9 | 1.3 | 0.6 | 0.5 | 0.1 |
| | G013967 | 92.2 | 0.8 | 88.7 | 0.8 | 67.3 | 3.9 | 38.0 | 1.9 | 15.3 | 1.3 | 4.3 | 1.0 | 1.5 | 0.2 | 0.4 | 0.1 |
| | G013971 | 95.5 | 1.0 | 94.4 | 1.2 | 74.2 | 2.1 | 45.1 | 2.4 | 17.2 | 3.2 | 4.5 | 1.4 | 1.0 | 0.1 | 0.4 | 0.1 |

TABLE 18
Editing in PHH with modified guides targeting SerpinA1.

| guide (nM) | | 33.3 | | 11.1 | | 3.7 | | 1.23 | | 0.41 | | 0.14 | | 0.05 | |
|---|---|---|---|---|---|---|---|---|---|---|---|---|---|---|---|
| | Guide | Edit | SD | Edit | SD | Edit | SD | Edit | SD | Edit | SD | Edit | SD | Edit | SD |
| PHH | G000480 | 96.6 | 0.1 | 95.5 | 0.1 | 94.7 | 0.7 | 91.9 | 0.4 | 85.2 | 0.6 | 70.4 | 4.7 | 41.4 | 1.8 |
| | G000486 | 95.0 | 0.8 | 91.8 | 0.6 | 87.1 | 0.6 | 71.7 | 1.6 | 47.3 | 4.5 | 22.3 | 2.1 | 8.5 | 1.3 |
| | G000407 | 77.3 | 1.7 | 79.9 | 3.7 | 66.7 | 7.4 | 48.0 | 2.1 | 27.2 | 5.3 | 12.0 | 1.1 | 5.2 | 0.6 |
| | G013954 | 76.2 | 4.2 | 73.1 | 6.4 | 65.1 | 7.0 | 39.2 | 8.9 | 22.5 | 3.3 | 10.9 | 2.7 | 4.4 | 0.8 |
| | G013959 | 73.6 | 0.4 | 77.3 | 2.9 | 69.1 | 1.3 | 48.8 | 5.5 | 29.5 | 6.7 | 13.4 | 4.3 | 7.0 | 1.3 |
| | G000415 | 93.4 | 0.8 | 91.6 | 1.3 | 90.6 | 1.2 | 86.6 | 1.7 | 77.0 | 3.3 | 56.7 | 6.8 | 34.7 | 4.3 |
| | G013958 | 93.6 | 0.1 | 92.1 | 1.8 | 89.4 | 1.3 | 85.5 | 1.5 | 73.8 | 2.7 | 52.4 | 5.9 | 28.7 | 2.6 |
| | G013963 | 90.9 | 0.1 | 90.7 | 1.3 | 91.7 | 1.8 | 88.4 | 1.1 | 83.6 | 1.5 | 68.6 | 7.2 | 37.4 | 6.8 |
| | G000408 | 91.7 | 2.4 | 88.0 | 0.0 | 84.3 | 1.5 | 74.6 | 2.7 | 48.8 | 6.3 | 21.7 | 2.5 | 6.7 | 0.7 |
| | G000413 | 49.3 | 59.1 | 85.1 | 60.2 | 77.9 | 55.1 | 58.1 | 41.1 | 31.1 | 22.0 | 14.8 | 10.5 | 5.1 | 3.6 |
| | G000409 | 93.2 | 0.6 | 91.1 | 2.3 | 86.9 | 0.4 | 80.9 | 1.4 | 57.7 | 2.9 | 31.9 | 0.8 | 9.3 | 0.4 |
| | G013955 | 95.3 | 0.8 | 93.1 | 1.3 | 88.0 | 1.2 | 82.5 | 0.4 | 63.4 | 2.3 | 37.8 | 0.1 | 13.4 | 0.8 |
| | G013960 | 94.8 | 0.1 | 91.6 | 1.8 | 89.5 | 1.2 | 85.9 | 0.6 | 73.6 | 4.5 | 43.4 | 0.6 | 19.3 | 0.5 |
| | G000412 | 96.0 | 67.9 | 88.6 | 62.6 | 88.1 | 62.3 | 80.8 | 57.1 | 68.8 | 48.6 | 30.6 | 21.6 | 9.0 | 6.4 |
| | G013956 | 46.0 | 65.1 | 91.0 | 0.4 | 89.7 | 63.4 | 83.1 | 58.8 | 64.2 | 45.4 | 30.4 | 21.5 | 15.2 | 10.7 |
| | G013961 | 95.0 | 67.2 | 92.6 | 65.5 | 90.8 | 64.2 | 79.3 | 56.1 | 51.3 | 36.3 | 20.8 | 14.7 | 7.0 | 4.9 |
| | G000414 | 95.0 | 0.4 | 91.6 | 1.6 | 89.8 | 0.4 | 82.9 | 0.2 | 63.6 | 1.7 | 34.0 | 1.7 | 14.0 | 0.3 |
| | G013962 | 57.6 | 53.5 | 48.5 | 58.7 | 89.0 | 62.9 | 80.8 | 57.1 | 62.7 | 44.3 | 14.4 | 20.3 | 14.7 | 10.4 |
| | G013957 | 92.3 | 65.3 | 46.0 | 62.4 | 88.8 | 62.8 | 80.9 | 57.2 | 60.4 | 42.7 | 30.3 | 21.4 | 13.9 | 9.8 |

TABLE 19
Editing in PCH with modified guides targeting SerpinA1.

| guide (nM) | | 33.3 | | 11.1 | | 3.7 | | 1.23 | | 0.41 | | 0.14 | | 0.05 | |
|---|---|---|---|---|---|---|---|---|---|---|---|---|---|---|---|
| | Guide | Edit | SD | Edit | SD | Edit | SD | Edit | SD | Edit | SD | Edit | SD | Edit | SD |
| PCH | G000480 | 19.0 | 1.9 | 3.7 | 0.9 | 1.2 | 0.3 | 0.3 | 0.1 | 0.1 | 0.0 | 0.1 | 0.0 | 0.1 | 0.0 |
| | G000502 | 82.8 | 9.8 | 73.0 | 10.2 | 61.8 | 11.0 | 41.2 | 2.3 | 18.5 | 1.2 | 6.3 | 0.4 | 3.0 | 0.2 |
| | G000407 | 64.4 | 9.0 | 48.2 | 6.7 | 27.8 | 7.4 | 13.3 | 0.8 | 5.4 | 0.1 | 1.9 | 0.9 | 0.6 | 0.1 |
| | G013954 | 52.3 | 16.3 | 39.3 | 6.4 | 23.4 | 7.4 | 10.8 | 2.1 | 4.4 | 3.1 | 2.1 | 1.6 | 1.1 | 0.1 |
| | G013959 | 65.6 | 9.8 | 46.3 | 9.4 | 31.4 | 7.2 | 15.7 | 4.5 | 4.9 | 0.8 | 2.2 | 0.1 | 1.2 | 0.6 |
| | G000415 | 87.0 | 7.0 | 74.2 | 5.7 | 59.3 | 7.4 | 32.4 | 3.5 | 10.3 | 2.4 | 4.3 | 0.5 | 1.7 | 0.1 |
| | G013958 | 90.7 | 4.9 | 77.0 | 9.3 | 60.3 | 7.5 | 33.7 | 0.2 | 11.9 | 0.7 | 4.8 | 1.1 | 1.9 | 0.5 |
| | G013963 | 89.2 | 4.9 | 83.5 | 2.2 | 71.9 | 4.7 | 47.5 | 1.8 | 19.9 | 3.0 | 7.7 | 1.1 | 2.5 | 0.4 |
| | G000408 | 22.4 | 0.2 | 7.1 | 0.6 | 1.7 | 0.1 | 0.6 | 0.1 | 0.1 | 0.1 | 0.0 | 0.0 | 0.1 | 0.1 |
| | G000413 | 7.0 | 0.1 | 1.7 | 0.1 | 0.4 | 0.1 | 0.1 | 0.0 | 0.1 | 0.0 | 0.2 | 0.0 | 0.2 | 0.1 |
| | G000409 | 71.9 | 0.5 | 46.8 | 0.7 | 14.7 | 0.3 | 3.4 | 0.0 | 1.2 | 0.1 | 0.2 | 0.1 | 0.2 | 0.1 |
| | G013955 | 85.0 | 4.1 | 60.7 | 1.1 | 35.2 | 2.8 | 10.8 | 0.8 | 3.4 | 1.4 | 1.4 | 0.6 | 0.3 | 0.1 |
| | G013960 | 86.4 | 0.8 | 63.9 | 0.6 | 37.5 | 2.0 | 12.4 | 3.6 | 4.6 | 2.3 | 1.0 | 0.5 | 0.3 | 0.3 |
| | G000412 | 82.7 | 6.0 | 57.1 | 0.5 | 34.1 | 1.4 | 11.0 | 2.6 | 4.4 | 1.5 | 1.2 | 0.8 | 0.1 | 0.0 |
| | G013956 | 81.9 | 2.0 | 64.2 | 0.9 | 40.7 | 1.6 | 18.8 | 1.2 | 7.3 | 0.7 | 1.3 | 0.8 | 0.4 | 0.1 |
| | G013961 | 72.8 | 18.2 | 70.6 | 3.6 | 43.3 | 3.5 | 22.8 | 3.9 | 6.1 | 1.7 | 1.4 | 0.1 | 0.4 | 0.1 |
| | G000414 | 76.5 | 1.6 | 42.2 | 3.3 | 25.1 | 3.4 | 8.1 | 0.1 | 2.8 | 0.5 | 0.8 | 0.6 | 0.1 | 0.0 |
| | G013957 | 82.3 | 5.2 | 50.6 | 7.0 | 26.4 | 2.1 | 10.2 | 1.0 | 3.8 | 0.4 | 1.2 | 0.3 | 1.2 | 0.3 |
| | G013962 | 76.8 | 0.4 | 46.1 | 1.5 | 24.3 | 2.5 | 10.2 | 1.8 | 2.8 | 0.6 | 0.9 | 0.1 | 0.6 | 0.2 |

### Example 10—In Vivo Studies of Short-sgRNA

LNPs prepared as described above in Example 1(F), comprising chemically synthesized sgRNAs (including short-sgRNAs) targeting the mouse TTR gene and IVT Cas9 mRNA in a 1:1 weight ratio, were administered to CD-1 female mice (N indicated below) or Sprague-Dawley female rats as described above in Example 1(H). Eight days post dose at necropsy, livers and blood were collected for NGS measurements of editing efficiency and serum TTR analysis, respectively, as described above in Example 1. Animals were weighed 24 hours post dose for overall wellness assessment.

FIGS. 1A and 1B show the editing efficiency and TTR protein levels, respectively, for LNPs containing the sgRNAs indicated in Tables 20A and 20B, which all target the same sequence in the TTR gene (See Table 1 for sgRNA nucleotide sequences). G000211 and G000282 served as reference comparators. The LNPs were made according to LNP Procedure B in Example 1(F). The data shown in FIGS. 1A and 1B are from mice administered 0.1 mg/kg (mpk) or 0.3 mg/kg of LNPs and are summarized in Tables 20A and 20B.

TABLE 20A

| Guide | Average Editing (%) | Std. Dev. | N |
|---|---|---|---|
| TSS | 0.14 | 0.05477 | 5 |
| G000282 - 0.3 mpk | 59.64 | 6.213 | 5 |
| G000282 - 0.1 mpk | 14.12 | 2.286 | 5 |
| G000515 - 0.3 mpk | 37.28 | 11.9 | 5 |
| G000515 - 0.1 mpk | 15.56 | 8.138 | 5 |
| G000621 - 0.3 mpk | 37.04 | 12.41 | 5 |
| G000621 - 0.1 mpk | 6.88 | 2.826 | 5 |
| G000632 - 0.3 mpk | 38.72 | 6.406 | 5 |
| G000632 - 0.1 mpk | 8.58 | 7.482 | 5 |
| G000638 - 0.3 mpk | 42.1 | 12.23 | 5 |
| G000638 - 0.1 mpk | 15.08 | 7.851 | 5 |
| G000639 - 0.3 mpk | 54.58 | 17.3 | 5 |
| G000639 - 0.1 mpk | 20.1 | 6.038 | 5 |
| G000640 - 0.3 mpk | 48.62 | 11.09 | 5 |
| G000640 - 0.1 mpk | 19.52 | 10.68 | 5 |
| G000641 - 0.3 mpk | 46.96 | 6.204 | 5 |
| G000641 - 0.1 mpk | 9.68 | 1.63 | 5 |
| G000211 - 0.3 mpk | 18.62 | 12.89 | 5 |
| G000211 - 0.1 mpk | 2.08 | 0.8044 | 5 |

TABLE 20B

| Guide | Average Serum TTR (μg/mL) | SD | N |
|---|---|---|---|
| TSS | 969.4 | 215.2 | 5 |
| G000282 - 0.3 mpk | 178.7 | 131.2 | 5 |
| G000282 - 0.1 mpk | 883.1 | 92.82 | 5 |
| G000515 - 0.3 mpk | 529.5 | 174.9 | 5 |
| G000515 - 0.1 mpk | 869.4 | 227.8 | 5 |
| G000621 - 0.3 mpk | 523 | 167.9 | 5 |
| G000261 - 0.1 mpk | 920.8 | 162.6 | 5 |
| G000632 - 0.3 mpk | 449.4 | 66.61 | 5 |
| G000632 - 0.1 mpk | 971.5 | 195.1 | 5 |
| G000638 - 0.3 mpk | 452.4 | 163.8 | 5 |
| G000638 - 0.1 mpk | 934 | 242.6 | 5 |
| G000639 - 0.3 mpk | 261.9 | 202.9 | 5 |
| G000639 - 0.1 mpk | 726.2 | 122.1 | 5 |
| G000640 - 0.3 mpk | 363.3 | 172 | 5 |
| G000640 - 0.1 mpk | 752.7 | 233.8 | 5 |
| G000641 - 0.3 mpk | 419 | 77.43 | 5 |
| G000641 - 0.1 mpk | 1018 | 58.16 | 5 |
| G000211 - 0.3 mpk | 692.4 | 157 | 5 |
| G000211 - 0.1 mpk | 970.3 | 113.7 | 5 |

FIGS. 2A and 2B show the editing efficiency and TTR protein levels, respectively, for LNPs containing the sgRNAs indicated in Tables 21A and 21B, which all target the same sequence in the TTR gene (See Table 1 for sgRNA nucleotide sequences). G000269 and G000283 served as reference comparators. The LNPs were made according to LNP Procedure B in Example 1(F). The data shown in FIGS. 2A and 2B are from mice administered 0.1 mg/kg (mpk) or 0.3 mg/kg of LNPs and are summarized in Tables 21A and 21B.

TABLE 21A

| Guide | Average Editing (%) | Std. Dev. | N |
|---|---|---|---|
| TSS | 0.12 | 0.0447214 | 5 |
| G000269 - 0.3 mpk | 8.94 | 2.65763 | 5 |
| G000269 - 0.1 mpk | 2.86 | 1.94756 | 5 |
| G000620 - 0.3 mpk | 24.62 | 5.66675 | 5 |
| G000620 - 0.1 mpk | 10.48 | 12.0136 | 5 |
| G000622 - 0.3 mpk | 14.08 | 3.20422 | 5 |
| G000622 - 0.1 mpk | 4.5 | 2.01122 | 5 |
| G000623 - 0.3 mpk | 14.8 | 9.48868 | 5 |
| G000623 - 0.1 mpk | 3.08 | 1.26768 | 5 |
| G000624 - 0.3 mpk | 32.86 | 12.3545 | 5 |
| G000624 - 0.1 mpk | 7.76 | 4.60196 | 5 |
| G000625 - 0.3 mpk | 41.28 | 6.99085 | 5 |
| G000625 - 0.1 mpk | 15.62 | 5.79974 | 5 |
| G000626 - 0.3 mpk | 37.7 | 13.1164 | 5 |
| G000626 - 0.1 mpk | 7.42 | 3.25146 | 5 |
| G000627 - 0.3 mpk | 27.86 | 12.5293 | 5 |
| G000627 - 0.1 mpk | 6.46 | 1.60094 | 5 |
| G000283 - 0.3 mpk | 40.14 | 6.59416 | 5 |
| G000283 - 0.1 mpk | 19.42 | 12.1568 | 5 |

TABLE 21B

| Guide | Average Serum TTR (μg/mL) | Std. Dev. | N |
|---|---|---|---|
| TSS | 970.798 | 154.875 | 5 |
| G000269 - 0.3 mpk | 859.012 | 244.538 | 5 |
| G000269 - 0.1 mpk | 769.096 | 101.675 | 5 |
| G000620 - 0.3 mpk | 595.108 | 218.142 | 5 |
| G000620 - 0.1 mpk | 614.304 | 117.668 | 5 |
| G000622 - 0.3 mpk | 537.89 | 35.5731 | 5 |
| G000622 - 0.1 mpk | 816.786 | 190.52 | 5 |
| G000623 - 0.3 mpk | 515.142 | 189.776 | 5 |
| G000623 - 0.1 mpk | 713.03 | 158.231 | 5 |
| G000624 - 0.3 mpk | 352.896 | 157.573 | 5 |
| G000624 - 0.1 mpk | 584.678 | 143.396 | 5 |
| G000625 - 0.3 mpk | 329.386 | 72.7329 | 5 |
| G000625 - 0.1 mpk | 595.212 | 90.3979 | 5 |
| G000626 - 0.3 mpk | 328.34 | 142.975 | 5 |
| G000626 - 0.1 mpk | 649.298 | 72.829 | 5 |
| G000627 - 0.3 mpk | 443.848 | 156.222 | 5 |
| G000627 - 0.1 mpk | 692.942 | 187.783 | 5 |
| G000283 - 0.3 mpk | 315.128 | 112.059 | 5 |
| G000283 - 0.1 mpk | 535.656 | 186.657 | 5 |

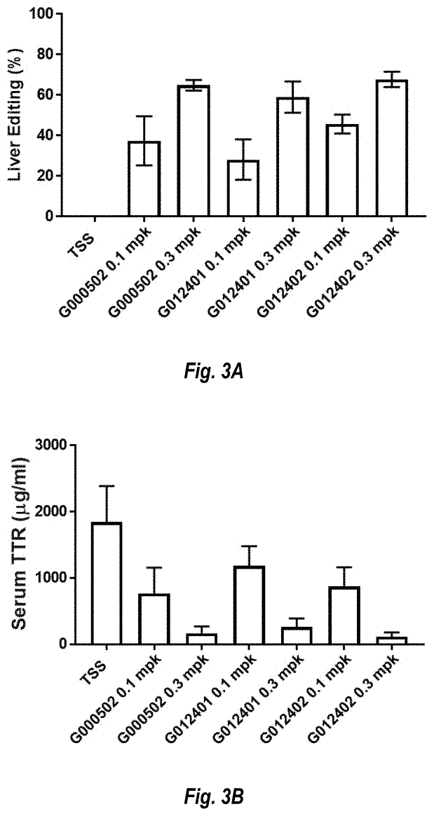

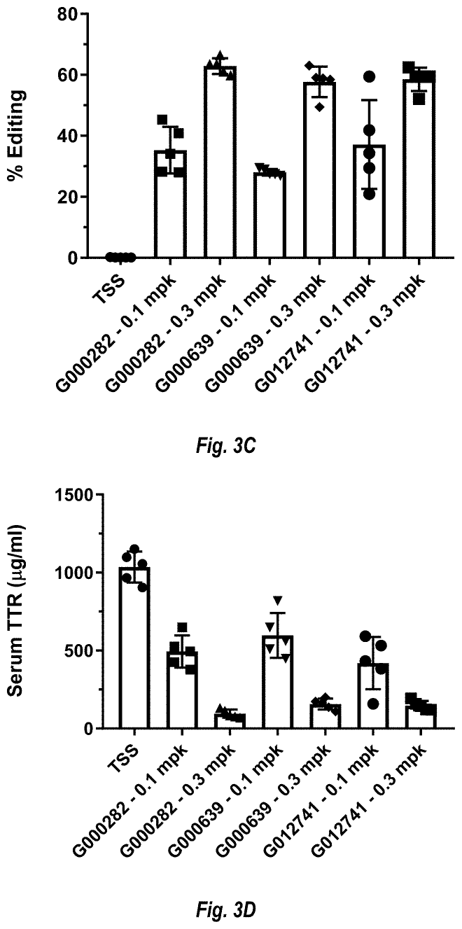

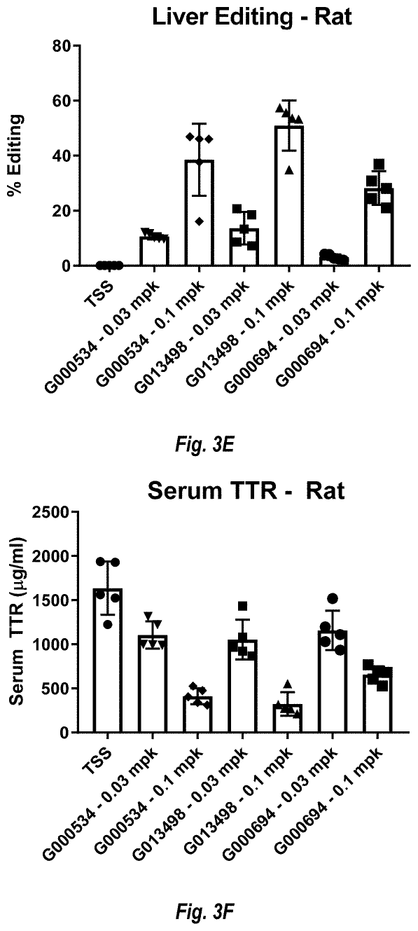

FIGS. 3A and 3B show the editing efficiency and TTR protein levels, respectively, for LNPs containing the sgRNAs indicated in Tables 22A and 22B, which all target the same sequence in the TTR gene (See Table 1 for sgRNA nucleotide sequences). G000502 served as a reference comparator. The LNPs were made according to LNP Procedure C in Example 1(F). The data shown in FIGS. 3A and 3B are from mice administered 0.1 mg/kg (mpk) or 0.3 mg/kg of LNPs and are summarized in Tables 22A and 22B.

TABLE 22A

| Guide | Average Editing (%) | Std. Dev. | N |
|---|---|---|---|
| TSS | 0.133333 | 0.057735 | 3 |
| G000502 0.1 mpk | 37.4 | 12.106 | 5 |
| G000502 0.3 mpk | 64.86 | 2.62545 | 5 |
| G009571 0.1 mpk | 47.6 | 6.98665 | 4 |
| G009571 0.3 mpk | 69.8 | 1.59217 | 5 |
| G010015 0.1 mpk | 47.86 | 6.09451 | 5 |
| G010015 0.3 mpk | 69.325 | 2.20662 | 4 |

TABLE 22B

| | Average Serum | | |
|---|---|---|---|
| Guide | TTR (μg/mL) | Std. Dev. | N |
| TSS | 1844.59 | 542.644 | 5 |
| G000502 0.1 mpk | 768.714 | 390.311 | 5 |
| G000502 0.3 mpk | 169.707 | 102.03 | 5 |
| G009571 0.1 mpk | 658.269 | 303.19 | 5 |
| G009571 0.3 mpk | 84.6392 | 33.3813 | 5 |
| G010015 0.1 mpk | 602.506 | 354.455 | 5 |
| G010015 0.3 mpk | 86.236 | 38.391 | 5 |

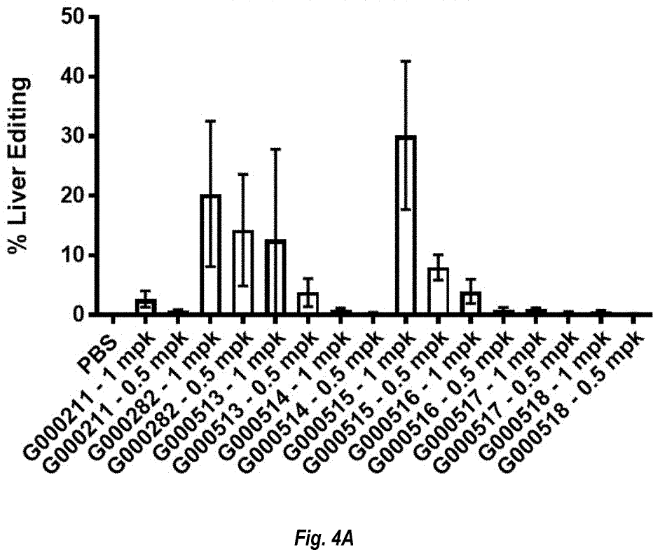

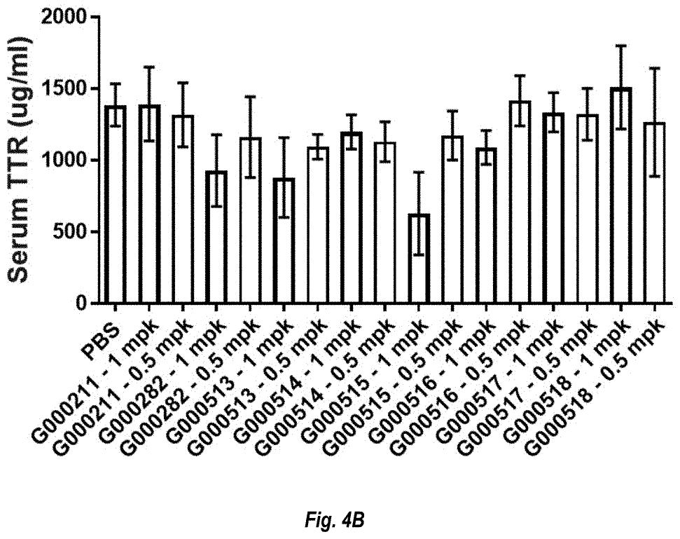

FIGS. 4A and 4B show the editing efficiency and TTR protein levels, respectively, for LNPs containing the sgRNAs indicated in Tables 23A and 23B, which all target the same sequence in the TTR gene (See Table 1 for sgRNA nucleotide sequences). G000211 and G000282 served as reference comparators. The LNPs were made according to LNP Procedure A in Example 1(F) using IVT Cas9 mRNA corresponding to SEQ ID NO: 501. The data shown in FIGS. 4A and 4B are from mice administered 0.5 mg/kg (mpk) or 1.0 mg/kg of LNPs and are summarized in Tables 23A and 23B.

TABLE 23A

| Guide | Average Editing (%) | Std. Dev. | N |
|---|---|---|---|
| PBS | 0.08 | 0.04472 | 5 |
| G000513 - 1 mpk | 12.7 | 15.11 | 5 |
| G000513 - 0.5 mpk | 3.76 | 2.359 | 5 |
| G000514 - 1 mpk | 0.82 | 0.3271 | 5 |
| G000514 - 0.5 mpk | 0.34 | 0.08944 | 5 |
| G000515 - 1 mpk | 30.12 | 12.44 | 5 |
| G000515 - 0.5 mpk | 7.98 | 2.111 | 5 |
| G000516 - 1 mpk | 3.96 | 2.035 | 5 |
| G000516 - 0.5 mpk | 0.92 | 0.3564 | 5 |
| G000517 - 1 mpk | 0.96 | 0.2302 | 5 |
| G000517 - 0.5 mpk | 0.42 | 0.1304 | 5 |
| G000518 - 1 mpk | 0.62 | 0.1304 | 5 |
| G000518 - 0.5 mpk | 0.22 | 0.04472 | 5 |
| G000211 - 1 mpk | 2.66 | 1.346 | 5 |
| G000211 - 0.5 mpk | 0.74 | 0.1517 | 5 |
| G000282 - 1 mpk | 20.3 | 12.21 | 5 |
| G000282 - 0.5 mpk | 14.24 | 9.371 | 5 |

TABLE 23B

| Guide | Average Serum TTR (μg/mL) | Std. Dev. | N |
|---|---|---|---|
| PBS | 1386 | 147.5 | 5 |
| G000513 - 1 mpk | 880.9 | 278.6 | 5 |
| G000513 - 0.5 mpk | 1095 | 86.52 | 5 |
| G000514 - 1 mpk | 1199 | 119.3 | 5 |
| G000514 - 0.5 mpk | 1131 | 139 | 4 |
| G000515 - 1 mpk | 629 | 288 | 5 |
| G000515 - 0.5 mpk | 1173 | 170.7 | 5 |
| G000516 - 1 mpk | 1091 | 118.6 | 5 |
| G000516 - 0.5 mpk | 1416 | 174.7 | 5 |
| G000517 - 1 mpk | 1336 | 137.2 | 5 |
| G000517 - 0.5 mpk | 1321 | 181.3 | 5 |
| G000518 - 1 mpk | 1508 | 289.9 | 5 |
| G000518 - 0.5 mpk | 1267 | 376.7 | 5 |
| G000211 - 1 mpk | 1393 | 256.9 | 5 |
| G000211 - 0.5 mpk | 1318 | 223.3 | 5 |
| G000282 - 1 mpk | 929.2 | 250.4 | 5 |
| G000282 - 0.5 mpk | 1162 | 282 | 5 |

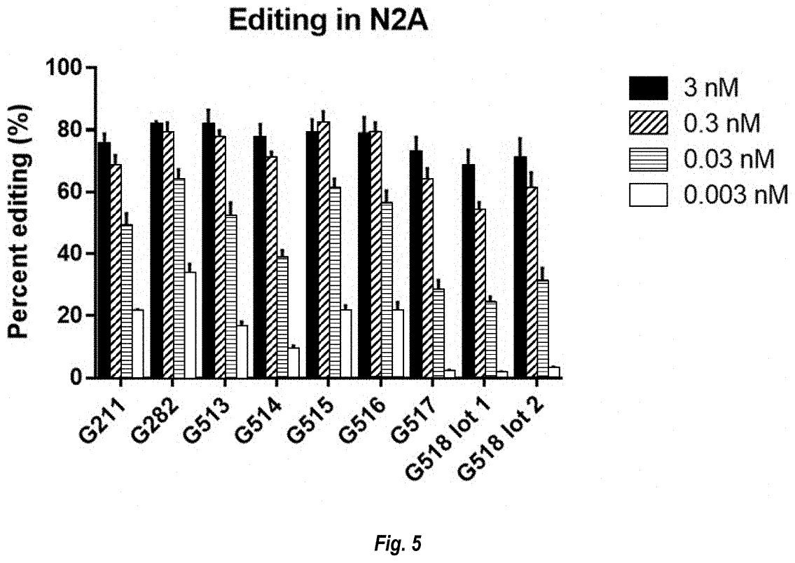

The same sgRNAs listed in Tables 23A and 23B (Table 1) were tested in vitro by transfection into Neuro2A cells, as per Example 1(C). Results are shown in FIG. 5.

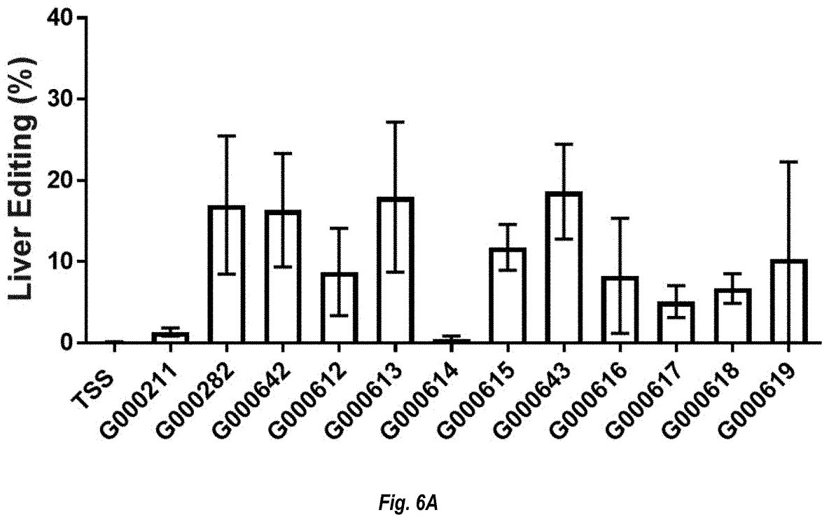

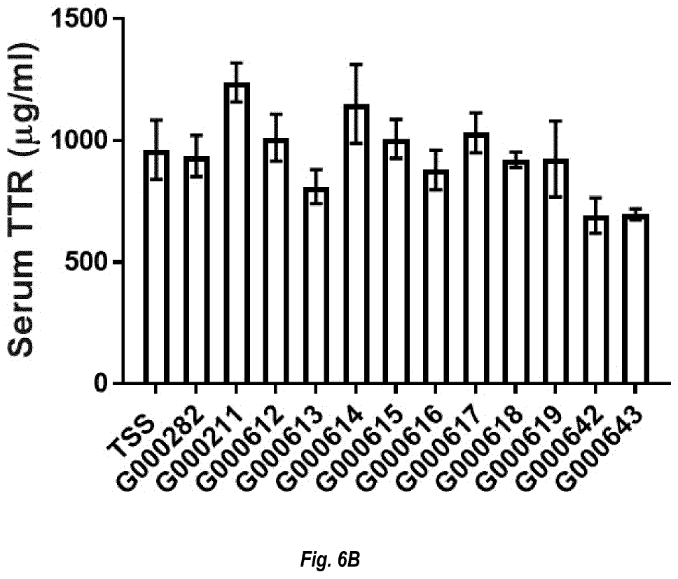

FIGS. 6A and 6B show the editing efficiency and TTR protein levels, respectively, for LNPs containing the sgRNAs indicated in Tables 24A and 24B, which all target the same sequence in the TTR gene (See Table 1 for sgRNA nucleotide sequences). G000211 and G000282 served as reference comparators. The LNPs were made according to LNP Procedure B in Example 1(F). The data shown in FIGS. 6A and 6B are from mice administered 0.1 mg/kg (mpk) of LNPs and are summarized in Tables 24A and 24B.

TABLE 24A

| Guide | Average Editing (%) | SD | N |
|---|---|---|---|
| TSS | 0.12 | 0.0447214 | 5 |
| G000282 | 16.98 | 8.49453 | 5 |
| G000211 | 1.34 | 0.497996 | 5 |
| G000612 | 8.74 | 5.38266 | 5 |
| G000613 | 17.96 | 9.23488 | 5 |
| G000614 | 0.5 | 0.339116 | 5 |
| G000615 | 11.76 | 2.81034 | 5 |
| G000616 | 8.28 | 7.08428 | 5 |
| G000617 | 5.08 | 1.96647 | 5 |
| G000618 | 6.7 | 1.81934 | 5 |
| G000619 | 10.32 | 11.9485 | 5 |
| G000642 | 16.34 | 6.9877 | 5 |
| G000643 | 18.62 | 5.83841 | 5 |

TABLE 24B

| Guide | Average Serum TTR (μg/mL) | SD | N |
|---|---|---|---|
| TSS | 962.636 | 271.526 | 5 |
| G000282 | 936.624 | 190.211 | 5 |
| G000211 | 1239.13 | 179.194 | 5 |
| G000612 | 1011.85 | 215.796 | 5 |
| G000613 | 810.72 | 156.082 | 5 |
| G000614 | 1150.81 | 362.492 | 5 |
| G000615 | 1007.04 | 179.37 | 5 |
| G000616 | 879.592 | 180.917 | 5 |
| G000617 | 1031.62 | 184.594 | 5 |
| G000618 | 921.65 | 71.1735 | 5 |
| G000619 | 924.728 | 348.938 | 5 |
| G000642 | 692.038 | 162.344 | 5 |
| G000643 | 696.416 | 51.8907 | 5 |

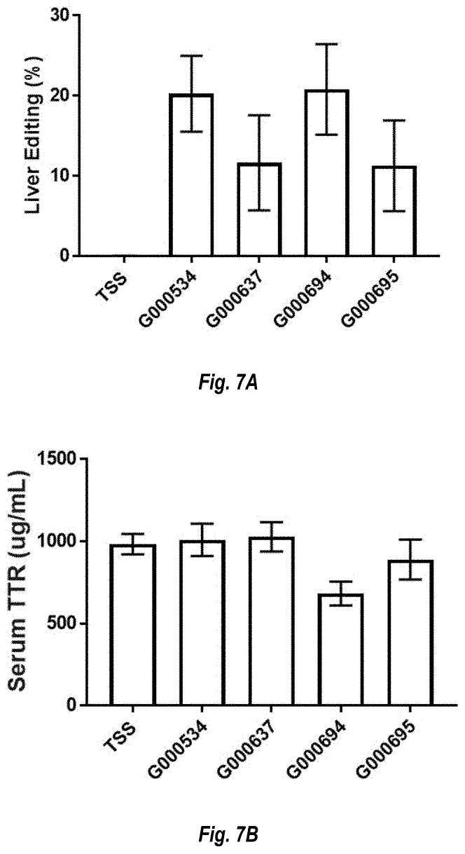

FIGS. 7A and 7B show the editing efficiency and TTR protein levels, respectively, for LNPs containing the sgRNAs indicated in Tables 25A and 25B, which all target the same sequence in the TTR gene (See Table 1 for sgRNA nucleotide sequences). G000534 served as a reference comparator. The LNPs were made according to LNP Procedure C in Example 1(F). The data shown in FIGS. 7A and 7B are from rats administered 0.3 mg/kg (mpk) of LNPs and are summarized in Tables 25A and 25B.

TABLE 25A

| Guide | Average Editing (%) | SD | N |
|---|---|---|---|
| TSS | 0.02 | 0.0447214 | 5 |
| G000534 | 20.26 | 4.73477 | 5 |
| G000637 | 11.66 | 5.93827 | 5 |
| G000694 | 20.8 | 5.62894 | 5 |
| G000695 | 11.28 | 5.66719 | 5 |

TABLE 25B

| Guide | Average Serum TTR (μg/mL) | SD | N |
|---|---|---|---|
| TSS | 983.078 | 138.405 | 5 |
| G000534 | 1008.92 | 220.498 | 5 |
| G000637 | 1027.73 | 199.071 | 5 |
| G000694 | 682.964 | 163.519 | 5 |
| G000695 | 889.5 | 270.242 | 5 |

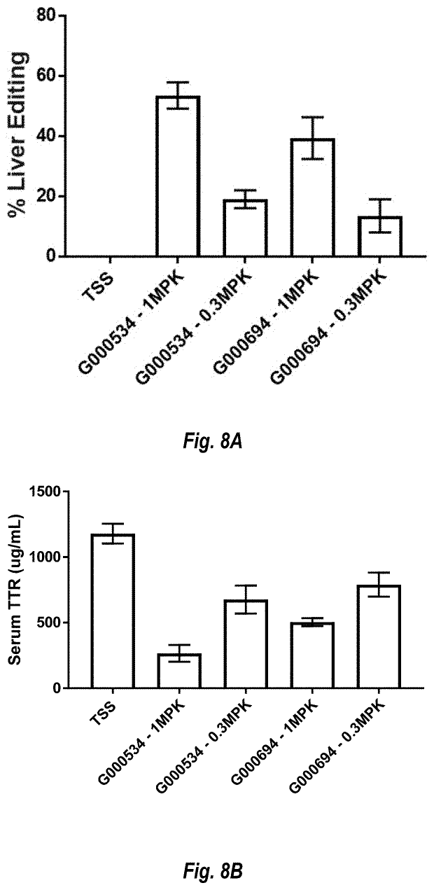

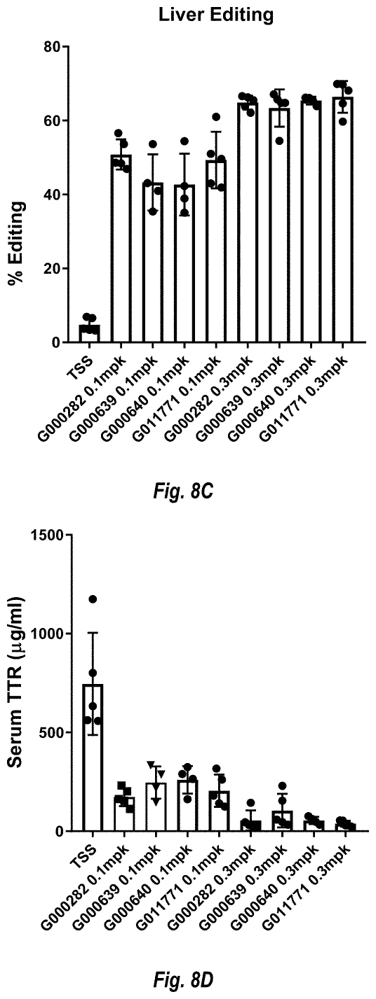

FIGS. 8A and 8B show the editing efficiency and TTR protein levels, respectively, for LNPs containing the sgRNAs indicated in Tables 26A and 26B, which all target the same sequence in the TTR gene (See Table 1 for sgRNA nucleotide sequences). G000534 served as a reference comparator. The LNPs were made according to LNP Procedure C in Example 1(F). The data shown in FIGS. 8A and 8B are from rats administered 0.3 mg/kg (mpk) and 1.0 mg/kg of LNPs and are summarized in Tables 26A and 26B.

TABLE 26A

| Guide | Average Editing (%) | SD | N |
|---|---|---|---|
| TSS | 0.02 | 0.0447214 | 5 |
| G000534 - 1 MPK | 53.48 | 4.34879 | 5 |
| G000534 - 0.3 MPK | 19 | 2.96395 | 5 |
| G000694 - 1 MPK | 39.32 | 6.93087 | 5 |
| G000694 - 0.3 MPK | 13.46 | 5.50391 | 5 |

TABLE 26B

| Guide | Average Serum TTR (μg/mL) | SD | N |
|---|---|---|---|
| TSS | 1178.71 | 75.8721 | 5 |
| G000534 - 1 MPK | 266.136 | 63.6724 | 5 |
| G000534 - 0.3 MPK | 676.446 | 107.07 | 5 |
| G000694 - 1 MPK | 503.888 | 30.8714 | 5 |
| G000694 - 0.3 MPK | 789.686 | 91.9034 | 5 |

### Example 11—In Vivo Studies of sgRNAs

LNPs prepared as described above in Example 1(F) (LNP Procedure D), comprising chemically synthesized sgRNAs targeting the mouse TTR gene and IVT Cas9 mRNA in a 1:1 weight ratio, were administered to CD-1 female mice (N indicated below) as described above in Example 1(E). Eight days post dose at necropsy, livers and blood were collected for NGS measurements of editing efficiency and serum TTR analysis, respectively, as described above in Example 1. Animals were weighed 24 hours post dose for overall wellness assessment.

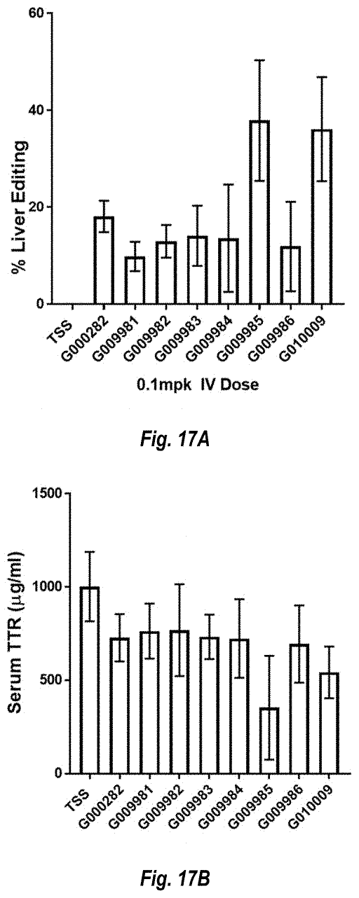

FIGS. 17A and 17B show the editing efficiency and TTR protein levels, respectively, for LNPs containing G282, G9981-G9986, and G10009, which all have the same nucleotide sequence as G282. The data shown in FIGS. 17A and 17B are from mice administered 0.1 mg/kg of LNPs, and are summarized in Tables 27A and 27B.

TABLE 27A

| Guide ID | Average % Editing | Std. Dev. | N |
|---|---|---|---|
| TSS | 0.1 | 0 | 5 |
| G000282 | 18.12 | 3.27826 | 5 |
| G009981 | 9.84 | 3.01297 | 5 |
| G009982 | 12.94 | 3.3709 | 5 |
| G009983 | 14.08 | 6.19007 | 5 |
| G009984 | 13.62 | 11.0981 | 5 |
| G009985 | 37.9 | 12.406 | 5 |
| G009986 | 11.9 | 9.25122 | 5 |
| G010009 | 36.14 | 10.7162 | 5 |

TABLE 27B

| Guide ID | Serum TTR (μg/mL) | Std. Dev. | N |
|---|---|---|---|
| TSS | 1002.47 | 185.909 | 5 |
| G000282 | 727.622 | 126.773 | 5 |
| G009981 | 764.096 | 147.486 | 5 |
| G009982 | 768.886 | 246.038 | 5 |
| G009983 | 733.138 | 119.212 | 5 |
| G009984 | 724.15 | 210.047 | 5 |
| G009985 | 353.746 | 277.917 | 5 |
| G009986 | 694.538 | 206.566 | 5 |
| G010009 | 542.53 | 138.99 | 5 |

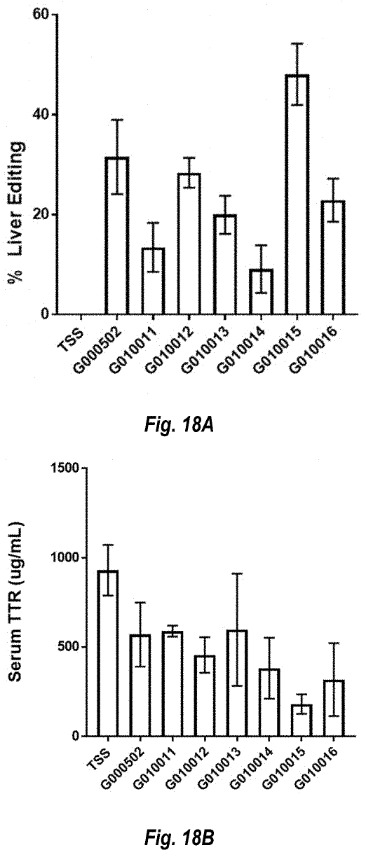

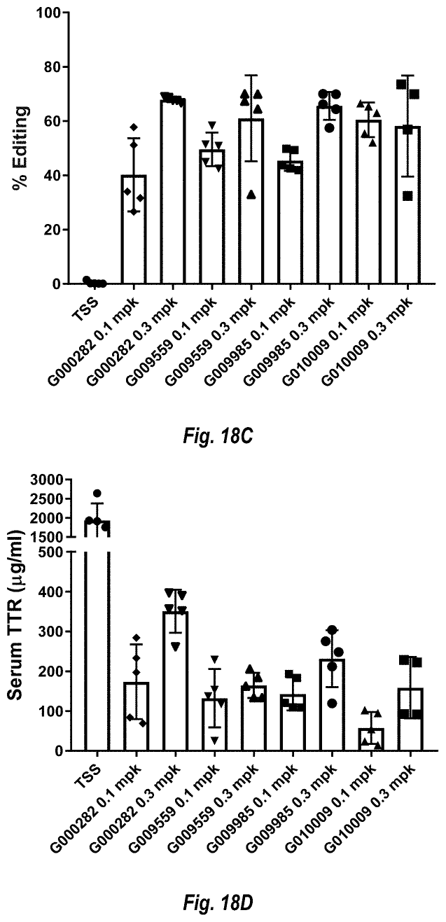

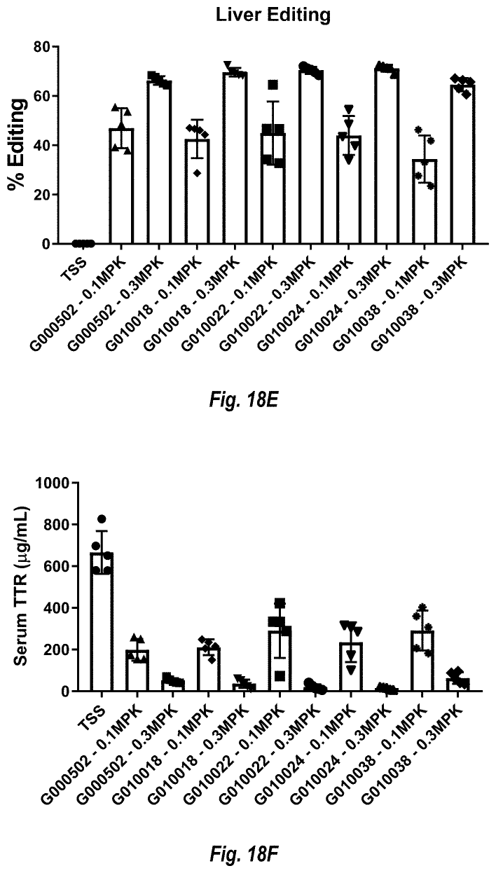

FIGS. 18A and 18B show the editing efficiency and TTR protein levels, respectively, for LNPs containing G502 and G10011-G10016, which all have the same nucleotide sequence as G502. The data shown in FIGS. 18A and 18B are from mice administered 0.1 mg/kg of LNPs, and are summarized in Tables 28A and 28B.

TABLE 28A

| Guide ID | Average % Editing | Std. Dev. | N |
|---|---|---|---|
| TSS | 0.1 | 0 | 5 |
| G000502 | 31.58 | 7.40993 | 5 |
| G010011 | 13.44 | 4.90184 | 5 |
| G010012 | 28.42 | 2.98613 | 5 |
| G010013 | 20 | 3.83536 | 5 |
| G010014 | 9.12 | 4.75994 | 5 |
| G010015 | 48.08 | 6.12552 | 5 |
| G010016 | 22.92 | 4.32169 | 5 |

TABLE 28B

| Guide ID | Serum TTR (μg/ML) | Std. Dev. | N |
|---|---|---|---|
| TSS | 931.178 | 141.193 | 5 |
| G000502 | 570.332 | 179.698 | 5 |
| G010011 | 589.487 | 31.5553 | 4 |
| G010012 | 454.848 | 99.7835 | 5 |
| G010013 | 597.594 | 314.78 | 5 |
| G010014 | 381.214 | 169.623 | 5 |
| G010015 | 181.184 | 54.3217 | 5 |
| G010016 | 317.66 | 203.218 | 5 |

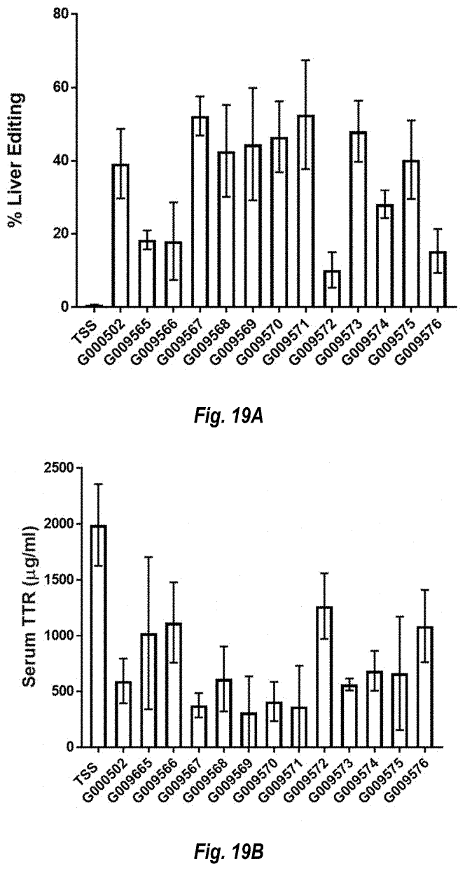

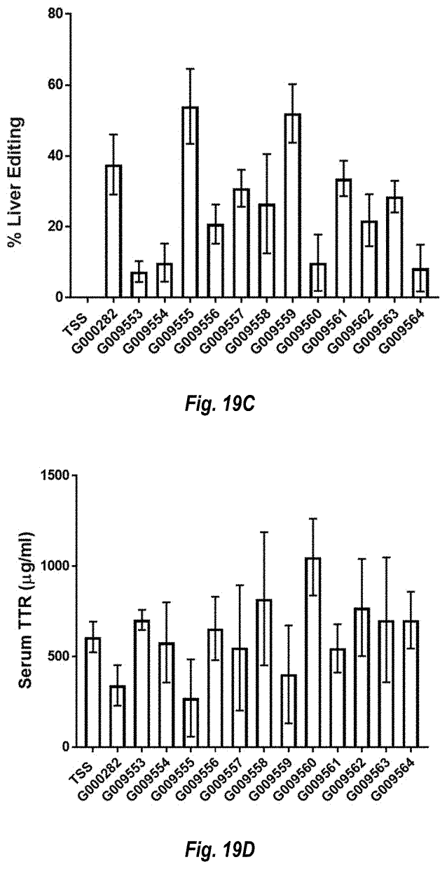

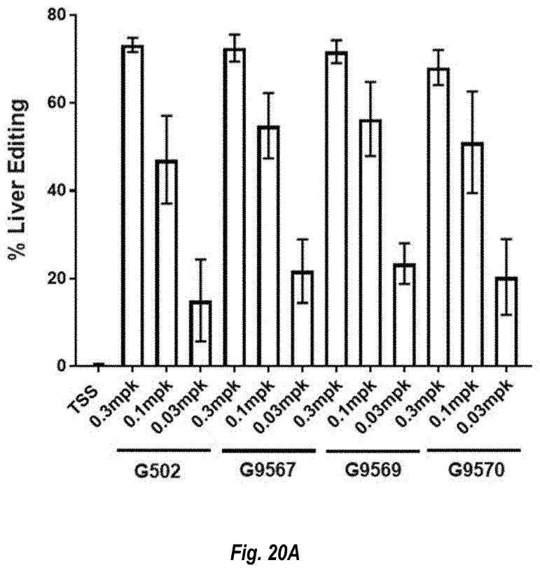

FIGS. 19A and 19B show the editing efficiency and TTR protein levels, respectively, for LNPs containing G502 and G9965-G9976, which all have the same nucleotide sequence as G502. The data shown in FIGS. 19A and 19B are from mice administered 0.1 mg/kg of LNPs, and are summarized in Tables 29A and 29B.

TABLE 29A

| Guide ID | Average % Editing | Std. Dev. | N |
|---|---|---|---|
| TSS | 0.6 | 0.0816497 | 4 |
| G000502 | 39.18 | 9.50616 | 5 |
| G009565 | 18.32 | 2.58592 | 5 |
| G009566 | 18 | 10.5654 | 4 |
| G009567 | 52.22 | 5.29641 | 5 |
| G009568 | 42.64 | 12.5763 | 5 |
| G009569 | 44.525 | 15.3743 | 4 |
| G009570 | 46.52 | 9.6699 | 5 |
| G009571 | 52.58 | 14.8569 | 5 |
| G009572 | 10.18 | 4.80749 | 5 |
| G009573 | 48.04 | 8.35063 | 5 |
| G009574 | 28.1 | 3.80592 | 5 |
| G009575 | 40.26 | 10.7549 | 5 |
| G009576 | 15.36 | 6.01731 | 5 |

TABLE 29B

| Guide ID | Serum TTR (μg/mL) | Std. Dev. | N |
|---|---|---|---|
| TSS | 1992.98 | 364.252 | 5 |
| G000502 | 594.954 | 199.578 | 5 |
| G009565 | 1024.87 | 681.864 | 5 |
| G009566 | 1120.52 | 361.314 | 4 |
| G009567 | 378.082 | 108.791 | 5 |
| G009568 | 613.27 | 290.386 | 5 |
| G009569 | 312.825 | 324.585 | 4 |
| G009570 | 410.94 | 175.285 | 5 |
| G009571 | 365.506 | 365.327 | 5 |
| G009572 | 1267.14 | 295.1 | 5 |
| G009573 | 564.246 | 53.4768 | 5 |
| G009574 | 685.998 | 178.199 | 5 |
| G009575 | 664.69 | 507.897 | 5 |
| G009576 | 1087.78 | 325.185 | 5 |

FIGS. 19C and 19D show the editing efficiency and TTR protein levels, respectively, for LNPs containing G282 and G9553-G9564, which all have the same nucleotide sequence as G282. The data shown in FIGS. 19C and 19D are from mice administered 0.1 mg/kg of LNPs, and are summarized in Tables 30A and 30B.

TABLE 30A

| Guide ID | Average % Editing | Std. Dev. | N |
|---|---|---|---|
| TSS | 0.1 | 0 | 5 |
| G000282 | 37.56 | 8.50194 | 5 |
| G009553 | 7.35 | 2.93201 | 4 |
| G009554 | 9.85 | 5.35257 | 4 |
| G009555 | 54 | 10.5376 | 5 |
| G009556 | 20.72 | 5.53281 | 5 |
| G009557 | 30.86 | 5.2491 | 5 |
| G009558 | 26.5 | 14.046 | 5 |
| G009559 | 52 | 8.22283 | 5 |
| G009560 | 9.82 | 7.95217 | 5 |
| G009561 | 33.62 | 5.01568 | 5 |
| G009562 | 21.8 | 7.32427 | 5 |
| G009563 | 28.5 | 4.48497 | 5 |
| G009564 | 8.3 | 6.59735 | 5 |

TABLE 30B

| Guide ID | Serum TTR (μg/mL) | Std. Dev. | N |
|---|---|---|---|
| TSS | 609.04 | 85.4341 | 5 |
| G000282 | 341.19 | 111.876 | 5 |
| G009553 | 704.38 | 55.5751 | 4 |
| G009554 | 578.958 | 222.003 | 5 |
| G009555 | 271.606 | 212.904 | 5 |
| G009556 | 656.606 | 176.012 | 5 |
| G009557 | 549.578 | 346.277 | 5 |
| G009558 | 820.098 | 368.242 | 5 |
| G009559 | 402.612 | 270.913 | 5 |
| G009560 | 1050.99 | 211.752 | 5 |
| G009561 | 546.352 | 134.462 | 5 |
| G009562 | 771.896 | 268.971 | 5 |
| G009563 | 703.896 | 345.506 | 5 |
| G009564 | 702.558 | 158.096 | 5 |

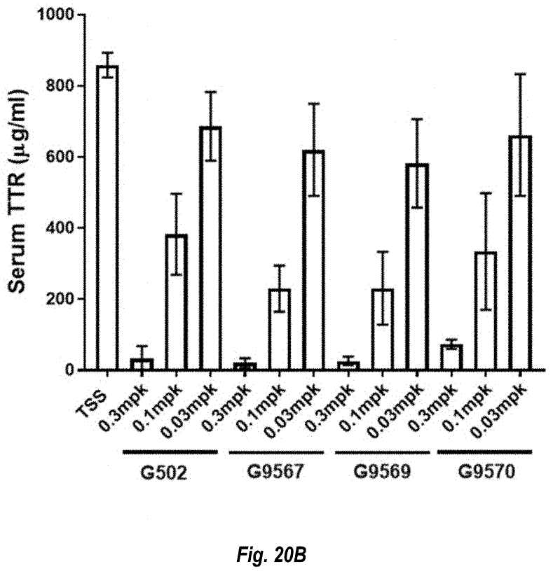

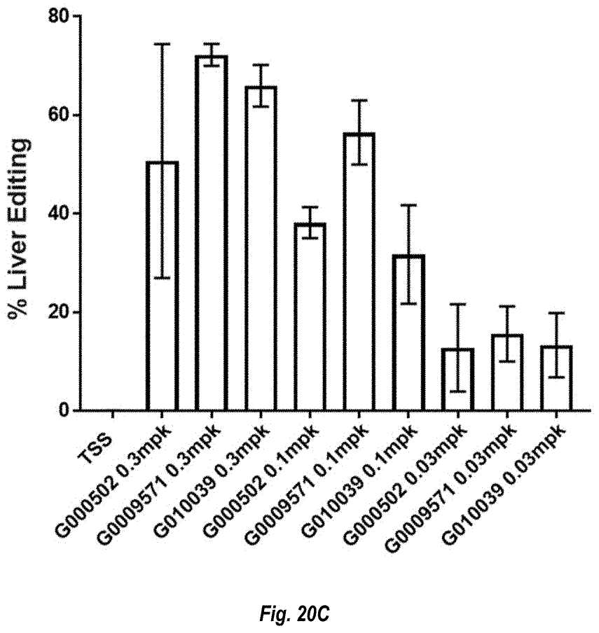

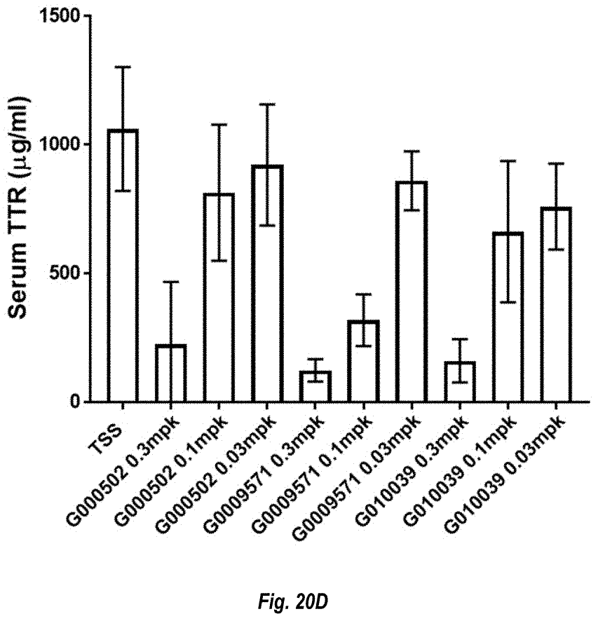

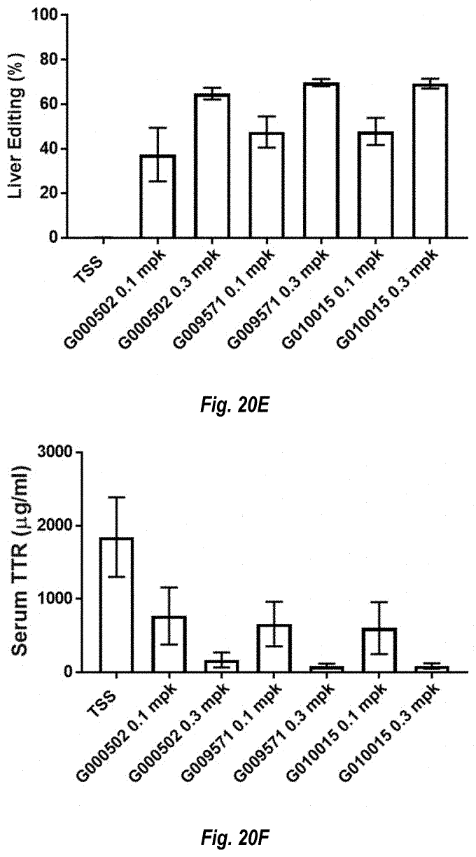

FIGS. 20A and 20B show the editing efficiency and TTR protein levels, respectively, for LNPs containing G502, G9567, G9569, and G9570, which all have the same nucleotide sequence as G502. The data shown in FIGS. 20A and 20B are from mice administered 0.03 mg/kg, 0.1 mg/kg, or 0.3 mg/kg of LNPs, and are summarized in Tables 31A and 31B.

TABLE 31A

| Guide ID | Average % Editing | Std. Dev. | N |
|---|---|---|---|
| TSS | 0.26 | 0.250998 | 5 |
| G000502 0.3 mpk | 73.26 | 1.61802 | 5 |
| G000502 0.1 mpk | 47.1 | 10.0135 | 5 |
| G000502 0.03 mpk | 15.02 | 9.31649 | 5 |
| G009567 0.3 mpk | 72.5 | 3.06105 | 5 |
| G009567 0.1 mpk | 54.86 | 7.43525 | 5 |
| G009567 0.03 mpk | 21.7 | 7.22565 | 5 |
| G009569 0.3 mpk | 71.7 | 2.60576 | 5 |
| G009569 0.1 mpk | 56.42 | 8.42033 | 5 |
| G009569 0.03 mpk | 23.42 | 4.62136 | 5 |
| G009570 0.3 mpk | 68.1 | 3.99166 | 4 |
| G009570 0.1 mpk | 51.08 | 11.5764 | 5 |
| G009570 0.03 mpk | 20.38 | 8.62827 | 5 |

TABLE 31B

| Guide ID | Serum TTR (μg/mL) | Std. Dev. | N |
|---|---|---|---|
| TSS | 858.846 | 34.7566 | 5 |
| G000502 0.3 mpk | 31.962 | 36.7047 | 5 |
| G000502 0.1 mpk | 382.614 | 113.613 | 5 |
| G000502 0.03 mpk | 686.612 | 96.3004 | 5 |
| G009567 0.3 mpk | 20.1267 | 13.6911 | 3 |
| G009567 0.1 mpk | 230.032 | 64.7601 | 5 |
| G009567 0.03 mpk | 620.4 | 130.126 | 5 |
| G009569 0.3 mpk | 25.91 | 11.9748 | 4 |
| G009569 0.1 mpk | 231.09 | 102.557 | 5 |
| G009569 0.03 mpk | 582.208 | 124.496 | 5 |
| G009570 0.3 mpk | 73.82 | 13.3713 | 5 |
| G009570 0.1 mpk | 334.308 | 163.522 | 5 |
| G009570 0.03 mpk | 661.48 | 171.449 | 5 |

FIGS. 20C and 20D show the editing efficiency and TTR protein levels, respectively, for LNPs containing G502, G9571, and G10039, which all have the same nucleotide sequence as G502. The data shown in FIGS. 20C and 20D are from mice administered 0.03 mg/kg, 0.1 mg/kg, or 0.3 mg/kg of LNPs, and are summarized in Tables 32A and 32B.

TABLE 32A

| Guide ID | Average % Editing | Std. Dev. | N |
|---|---|---|---|
| TSS | 0.08 | 0.0447214 | 5 |
| G000502 0.3 mpk | 50.7 | 23.7015 | 5 |
| G000502 0.1 mpk | 38.2 | 3.14245 | 5 |
| G000502 0.03 mpk | 12.78 | 8.84064 | 5 |
| G0009571 0.3 mpk | 72.24 | 2.21653 | 5 |
| G0009571 0.1 mpk | 56.48 | 6.50092 | 5 |
| G0009571 0.03 mpk | 15.6 | 5.6 | 5 |
| G010039 0.3 mpk | 65.98 | 4.22694 | 5 |
| G010039 0.1 mpk | 31.74 | 10.0179 | 5 |
| G010039 0.03 mpk | 13.34 | 6.50446 | 5 |

TABLE 32B

| Guide ID | Serum TTR (μg/mL) | Std. Dev. | N |
|---|---|---|---|
| TSS | 1062.23 | 240.945 | 5 |
| G000502 0.3 mpk | 224.37 | 242.604 | 4 |
| G000502 0.1 mpk | 814.642 | 264.733 | 5 |
| G000502 0.03 mpk | 922.306 | 235.495 | 5 |
| G0009571 0.3 mpk | 123.52 | 43.2127 | 4 |
| G0009571 0.1 mpk | 317.752 | 100.059 | 5 |
| G0009571 0.03 mpk | 860.7 | 114.188 | 5 |
| G010039 0.3 mpk | 160.613 | 83.6036 | 4 |
| G010039 0.1 mpk | 662.048 | 274.764 | 5 |
| G010039 0.03 mpk | 759.892 | 166.829 | 5 |

FIGS. 20E and 20F show the editing efficiency and TTR protein levels, respectively, for LNPs containing G502, G9571, and G10015, which all have the same nucleotide sequence as G502. The data shown in FIGS. 20E and 20F are from mice administered 0.1 mg/kg or 0.3 mg/kg of LNPs, and are summarized in Tables 33A and 33B.

TABLE 33A

| Guide ID | Average Editing (%) | Std. Dev. | N |
|---|---|---|---|
| TSS | 0.133333 | 0.057735 | 3 |
| G000502 0.1 mpk | 37.4 | 12.106 | 5 |
| G000502 0.3 mpk | 64.86 | 2.62545 | 5 |
| G009571 0.1 mpk | 47.6 | 6.98665 | 4 |
| G009571 0.3 mpk | 69.8 | 1.59217 | 5 |
| G010015 0.1 mpk | 47.86 | 6.09451 | 5 |
| G010015 0.3 mpk | 69.325 | 2.20662 | 4 |

TABLE 33B

| Guide ID | Serum TTR (μg/mL) | Std. Dev. | N |
|---|---|---|---|
| TSS | 1844.59 | 542.644 | 5 |
| G000502 0.1 mpk | 768.714 | 390.311 | 5 |
| G000502 0.3 mpk | 169.707 | 102.03 | 5 |
| G009571 0.1 mpk | 658.269 | 303.19 | 5 |
| G009571 0.3 mpk | 84.6392 | 33.3813 | 5 |
| G010015 0.1 mpk | 602.506 | 354.455 | 5 |
| G010015 0.3 mpk | 86.236 | 38.391 | 5 |

### Example 12—In Vivo Studies

FIGS. 8C and 8D show the editing efficiency and TTR protein levels, respectively, for LNPs containing the sgRNAs indicated in Table 34 (See Table 1 for sgRNA nucleotide sequences) which all target the same sequence in the TTR gene. G000282 served as a reference comparator. The LNPs were made according to LNP Procedure D in Example 1(F). The data shown in FIGS. 8C and 8D are from CD-1 mice administered 0.1 mg/kg and 0.3 mg/kg of total RNA and are summarized in Table 34.

TABLE 34
Liver Editing and Serum TTR

| | | | | | Serum | | | Serum |
|---|---|---|---|---|---|---|---|---|
| | Dose | % | | | TTR | | | TTR |
| Guide | (mpk) | Editing | SD | N | (μg/ml) | SD | N | % TSS |
| TSS | TSS | 4.8 | 1.8 | 5 | 746 | 259 | 5 | 100% |
| G000282 | 0.1 | 50.8 | 4.1 | 5 | 173 | 46 | 5 | 23% |
| G000282 | 0.3 | 64.9 | 1.9 | 5 | 55 | 51 | 5 | 7% |
| G000639 | 0.1 | 43.3 | 7.6 | 4 | 246 | 82 | 4 | 33% |
| G000639 | 0.3 | 63.4 | 5.1 | 5 | 104 | 85 | 5 | 14% |
| G000640 | 0.1 | 42.7 | 8.4 | 4 | 260 | 70 | 4 | 35% |
| G000640 | 0.3 | 65.4 | 1.1 | 4 | 55 | 19 | 4 | 7% |
| G011771 | 0.1 | 49.3 | 7.7 | 5 | 205 | 82 | 5 | 27% |
| G011771 | 0.3 | 66.4 | 4.3 | 5 | 38 | 15 | 5 | 5% |

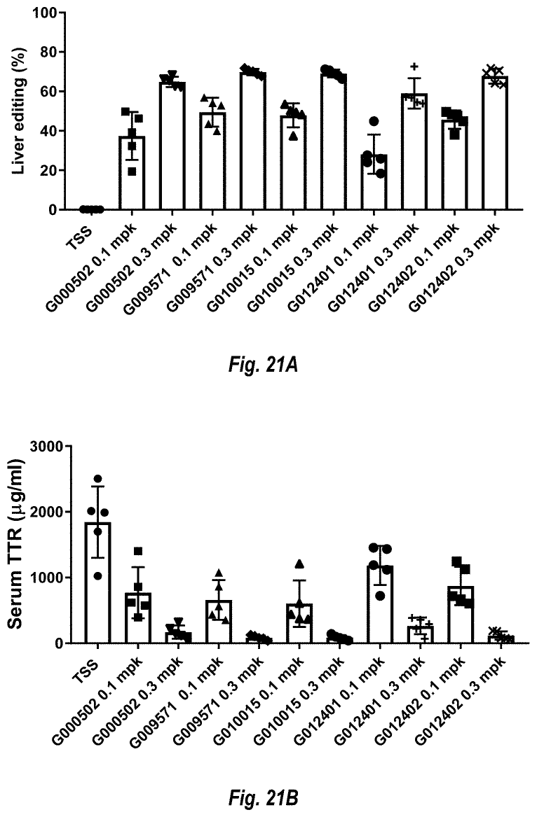

FIGS. 21A and 21B show the editing efficiency and TTR protein levels, respectively, for LNPs containing the sgRNAs indicated in Table 35 (See Table 1 for sgRNA nucleotide sequences) which all target the same sequence in the TTR gene. The LNPs were made according to LNP Procedure D in Example 1(F). The data shown in FIGS. 21A and 21B are from CD-1 female mice (n=5) administered 0.1 mg/kg and 0.3 mg/kg of total RNA and are summarized in Table 35.

TABLE 35
Liver Editing and Serum TTR

| | | | | Serum | | Serum |
|---|---|---|---|---|---|---|
| | Dose | % | | TTR | | TTR |
| Guide | (mpk) | Editing | SD | μg/ml | SD | % TSS |
| TSS | TSS | 0.1 | 0.0 | 1845 | 543 | 100% |
| G000502 | 0.1 | 37.4 | 12.1 | 769 | 390 | 42% |
| G000502 | 0.3 | 64.9 | 2.6 | 170 | 102 | 9% |
| G009571 | 0.1 | 49.5 | 7.3 | 658 | 303 | 36% |
| G009571 | 0.3 | 69.8 | 1.6 | 85 | 33 | 5% |
| G010015 | 0.1 | 47.9 | 6.1 | 603 | 354 | 33% |
| G010015 | 0.3 | 69.1 | 2.0 | 86 | 38 | 5% |
| G012401 | 0.1 | 28.2 | 10.0 | 1183 | 298 | 64% |
| G012401 | 0.3 | 59.0 | 7.7 | 264 | 127 | 14% |
| G012402 | 0.1 | 45.7 | 4.6 | 872 | 293 | 47% |
| G012402 | 0.3 | 67.8 | 3.8 | 117 | 64 | 6% |

FIGS. 18C-D show the editing efficiency and TTR protein levels, respectively, for LNPs containing the sgRNAs indicated in Table 36 (See Table 1 for sgRNA nucleotide sequences) which all target the same sequence in the TTR gene. The LNPs were made according to LNP Procedure D in Example 1(F). The data shown in FIGS. 18C-D are from CD-1 female mice administered 0.1 mg/kg (mpk) and 0.3 mg/kg of total RNA and are summarized in Table 36.

TABLE 36
Liver Editing and Serum TTR

| | | | | Serum | | | Serum |
|---|---|---|---|---|---|---|---|
| | Dose | % | | TTR | | | TTR |
| Guide | (mpk) | Editing | SD | (ug/ml) | SD | N | % TSS |
| TSS | TSS | 0.4 | 0.6 | 1935 | 443 | 5 | 100% |
| G000282 | 0.1 | 40.2 | 13.5 | 174 | 94 | 5 | 9% |
| G000282 | 0.3 | 67.9 | 0.9 | 351 | 54 | 5 | 18% |
| G009559 | 0.1 | 49.6 | 6.2 | 132 | 73 | 5 | 7% |
| G009559 | 0.3 | 61.0 | 15.8 | 165 | 32 | 5 | 9% |
| G009985 | 0.1 | 45.5 | 3.8 | 143 | 42 | 5 | 7% |
| G009985 | 0.3 | 65.6 | 5.1 | 232 | 71 | 5 | 12% |
| G010009 | 0.1 | 60.5 | 6.4 | 58 | 40 | 5 | 3% |
| G010009 | 0.3 | 58.2 | 18.6 | 159 | 77 | 4 | 8% |

FIGS. 18E-F show the editing efficiency and TTR protein levels, respectively, for LNPs containing the sgRNAs indicated in Table 37 (See Table 1 for sgRNA nucleotide sequences) which all target the same sequence in the TTR gene. The LNPs were made according to LNP Procedure D in Example 1(F). The data shown in FIGS. 18E-F are from CD-1 female mice (n=5) administered 0.1 mg/kg (mpk) and 0.3 mg/kg of total RNA and are summarized in Table 37.

TABLE 37
Liver Editing and Serum TTR

| | | | | Serum | | Serum |
|---|---|---|---|---|---|---|
| | Dose | % | | TTR | | TTR |
| Guide | (mpk) | Editing | SD | (ug/ml) | SD | % TSS |
| TSS | TSS | 0.1 | 0.0 | 666 | 103 | 100% |
| G000502 | 0.1 | 46.9 | 8.1 | 199 | 52 | 30% |
| G000502 | 0.3 | 66.3 | 1.7 | 51 | 12 | 8% |
| G010018 | 0.1 | 42.5 | 7.8 | 211 | 38 | 32% |
| G010018 | 0.3 | 69.6 | 1.7 | 36 | 20 | 5% |
| G010022 | 0.1 | 45.0 | 12.8 | 290 | 131 | 44% |
| G010022 | 0.3 | 70.4 | 1.4 | 20 | 13 | 3% |
| G010024 | 0.1 | 44.0 | 7.9 | 235 | 95 | 35% |
| G010024 | 0.3 | 71.2 | 1.4 | 14 | 6 | 2% |
| G010038 | 0.1 | 34.4 | 9.6 | 291 | 96 | 44% |
| G010038 | 0.3 | 64.6 | 2.7 | 63 | 27 | 9% |

FIGS. 3C-D show the editing efficiency and TTR protein levels, respectively, for LNPs containing the sgRNAs indicated in Table 38 (See Table 1 for sgRNA nucleotide sequences) which all target the same sequence in the TTR gene. The LNPs were made according to LNP Procedure D in Example 1(F). The data shown in FIGS. 3C-D are from CD-1 female mice (n=5) administered 0.1 mg/kg (mpk) and 0.3 mg/kg of total RNA and are summarized in Table 38.

TABLE 38
Liver Editing and Serum TTR

| | | | | Serum | | Serum |
|---|---|---|---|---|---|---|
| | Dose | % | | TTR | | TTR |
| Guide | (mpk) | Editing | SD | ug/ml | SD | % TSS |
| TSS | TSS | 0.1 | 0.0 | 1035 | 100 | 100% |
| G000282 | 0.1 | 35.3 | 7.7 | 494 | 103 | 48% |
| G000282 | 0.3 | 62.9 | 2.6 | 95 | 27 | 9% |
| G000639 | 0.1 | 28.0 | 1.1 | 597 | 144 | 58% |
| G000639 | 0.3 | 57.7 | 5.0 | 157 | 35 | 15% |
| G012741 | 0.1 | 37.2 | 14.6 | 420 | 167 | 41% |
| G012741 | 0.3 | 58.6 | 3.8 | 147 | 30 | 14% |

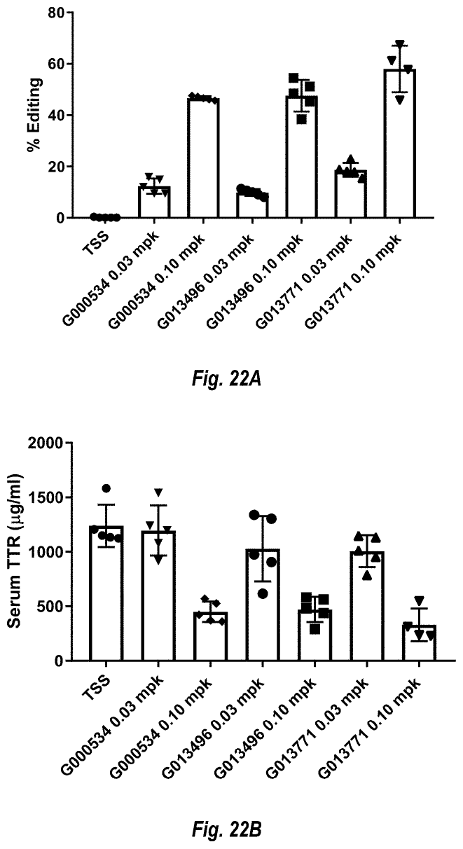

FIGS. 22A-B show the editing efficiency and TTR protein levels, respectively, for LNPs containing the sgRNAs indicated in Table 39 (See Table 1 for sgRNA nucleotide sequences) which all target the same sequence in the TTR gene. The LNPs were made according to LNP Procedure P4.3 in Example 1(F). The data shown in FIGS. 22A-B are from Sprague Dawley rats administered 0.1 mg/kg and 0.03 mg/kg of total RNA and are summarized in Table 39.

TABLE 39
Liver Editing and Serum TTR in rat

| | | | | Serum | | | Serum |
|---|---|---|---|---|---|---|---|
| | Dose | % | | TTR | | | TTR |
| Guide | (mpk) | Editing | SD | (ug/ml) | SD | N | % TSS |
| TSS | TSS | 0.2 | 0.1 | 1239 | 195 | 5 | 100% |
| G000534 | 0.03 | 12.4 | 3.0 | 1195 | 230 | 5 | 96% |
| G000534 | 0.10 | 46.6 | 0.77 | 450 | 94 | 5 | 36% |
| G013496 | 0.03 | 9.9 | 1.3 | 1028 | 300 | 5 | 83% |
| G013496 | 0.10 | 47.6 | 6.2 | 471 | 116 | 5 | 38% |
| G013771 | 0.03 | 18.7 | 2.7 | 1006 | 147 | 5 | 81% |
| G013771 | 0.10 | 58.0 | 9.1 | 331 | 150 | 4 | 27% |

Table 40 shows the editing efficiency and TTR protein levels, respectively, for LNPs containing the indicated sgRNAs (See Table 1 for sgRNA nucleotide sequences) which all target the same sequence in the TTR gene. The LNPs were made according to LNP Procedure D in Example 1(F). The data shown in Table 40 are from CD-1 female mice administered 0.1 mg/kg of total RNA.

TABLE 40
Liver Editing and Serum TTR

| | | | | Serum | | | Serum |
|---|---|---|---|---|---|---|---|
| | | | | TTR | | | TTR |
| Guide | Editing | SD | N | (μg/ml) | SD | N | % TSS |
| TSS | 0.3 | 0.1 | 5 | 759 | 106 | 5 | 100% |
| G000502 | 26.7 | 6.4 | 5 | 480 | 148 | 5 | 63% |
| G012421 | 17.2 | 4.9 | 5 | 642 | 137 | 5 | 85% |
| G012422 | 33.5 | 9.5 | 5 | 479 | 170 | 5 | 63% |
| G012423 | 12.4 | 2.7 | 5 | 626 | 82 | 5 | 82% |
| G012424 | 18.4 | 6.1 | 5 | 614 | 106 | 5 | 81% |
| G012426 | 31.4 | 4.8 | 5 | 481 | 122 | 5 | 63% |
| G012443 | 36.6 | 10.5 | 4 | 406 | 115 | 4 | 53% |
| G012448 | 37.6 | 6.6 | 5 | 400 | 194 | 5 | 53% |
| G012453 | 16.5 | 4.0 | 5 | 644 | 126 | 5 | 85% |
| G012456 | 3.6 | 1.3 | 5 | 795 | 89 | 5 | 105% |
| G012457 | 14.8 | 5.5 | 5 | 675 | 167 | 5 | 89% |

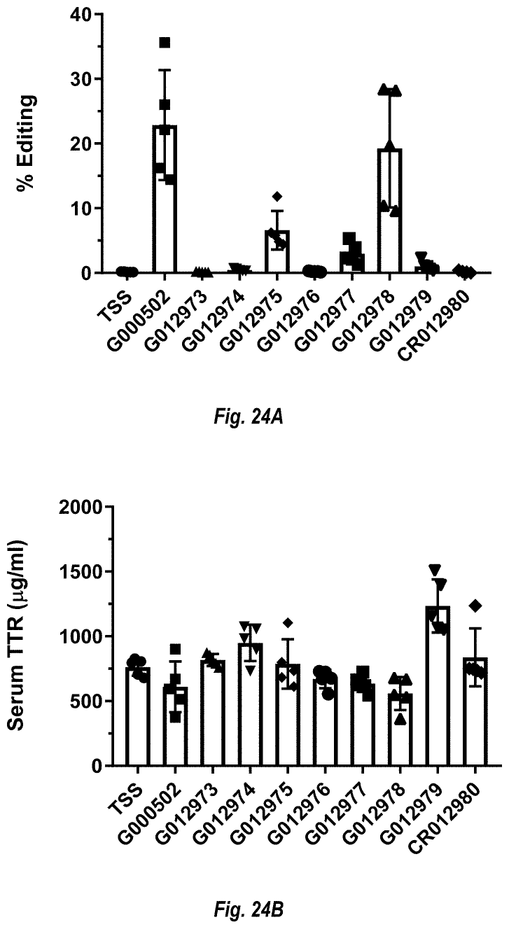

FIGS. 24A-B show the editing efficiency and TTR protein levels, respectively, for LNPs containing the sgRNAs indicated in Table 41 (See Table 1 for sgRNA nucleotide sequences) which all target the same sequence in the TTR gene. The LNPs were made according to LNP Procedure D in Example 1(F). The data shown in FIGS. 24A-B are from CD-1 female mice administered 0.1 mg/kg of total RNA and are summarized in Table 41.

TABLE 41
Liver Editing and Serum TTR

| | | | | Serum | | | Serum |
|---|---|---|---|---|---|---|---|
| | % | | | TTR | | | TTR |
| Guide | Editing | SD | N | (μg/ml) | SD | N | % TSS |
| TSS | 0.1 | 0.1 | 5 | 763 | 66 | 5 | 100% |
| G000502 | 22.9 | 8.5 | 5 | 610 | 196 | 5 | 80% |
| G012973 | 0.1 | 0.1 | 5 | 818 | 47 | 5 | 107% |
| G012974 | 0.5 | 0.2 | 5 | 949 | 140 | 5 | 124% |
| G012975 | 6.6 | 3.0 | 5 | 787 | 190 | 5 | 103% |
| G012976 | 0.2 | 0.1 | 5 | 671 | 71 | 5 | 88% |
| G012977 | 3.0 | 1.6 | 5 | 636 | 65 | 5 | 83% |
| G012978 | 19.3 | 9.2 | 5 | 559 | 128 | 5 | 73% |
| G012979 | 1.0 | 0.8 | 5 | 1234 | 206 | 5 | 162% |
| CR012980 | 0.2 | 0.2 | 4 | 837 | 223 | 5 | 110% |

FIGS. 3E-F show the editing efficiency and TTR protein levels, respectively, for LNPs containing the sgRNAs indicated in Table 42 (See Table 1 for sgRNA nucleotide sequences) which all target the same sequence in the TTR gene. The LNPs were made according to LNP Procedure D in Example 1(F). The data shown in FIGS. 3E-F are from Sprague Dawley female rats (n=5) administered 0.1 mg/kg and 0.03 mg/kg of total RNA and are summarized in Table 42.

TABLE 42
Liver editing & Serum TTR in Rat

| | | | | Serum | | Serum |
|---|---|---|---|---|---|---|
| | Dose | % | | TTR | | TTR |
| Guide | (mpk) | Editing | SD | ug/ml | SD | % TSS |
| TSS | TSS | 0.1 | 0.0 | 1635 | 301 | 100% |
| G013498 | 0.03 | 13.7 | 5.9 | 1054 | 225 | 64% |
| G013498 | 0.10 | 51.0 | 9.1 | 325 | 133 | 20% |
| G000534 | 0.03 | 10.7 | 1.1 | 1105 | 154 | 68% |
| G000534 | 0.10 | 38.6 | 13.1 | 411 | 90 | 25% |
| G000694 | 0.03 | 3.1 | 1.0 | 1157 | 223 | 71% |
| G000694 | 0.10 | 28.2 | 6.1 | 656 | 92 | 40% |

### Example 13—Correlations between In Vitro and In Vivo Editing with sgRNAs

Chemically synthesized sgRNAs (G502 and G9565-G9576) and IVT Cas9 mRNA were administered to primary hepatocytes as lipoplex transfections or LNP transfections as described in Example 1(D) and 1(F) (LNP Procedure D), respectively. Editing of the TTR gene was determined by NGS as described above in Example 1(G). The same sgRNAs were also administered to CD-1 female mice as described in Example 5, particularly the section describing data shown in FIGS. 19A and 19B.

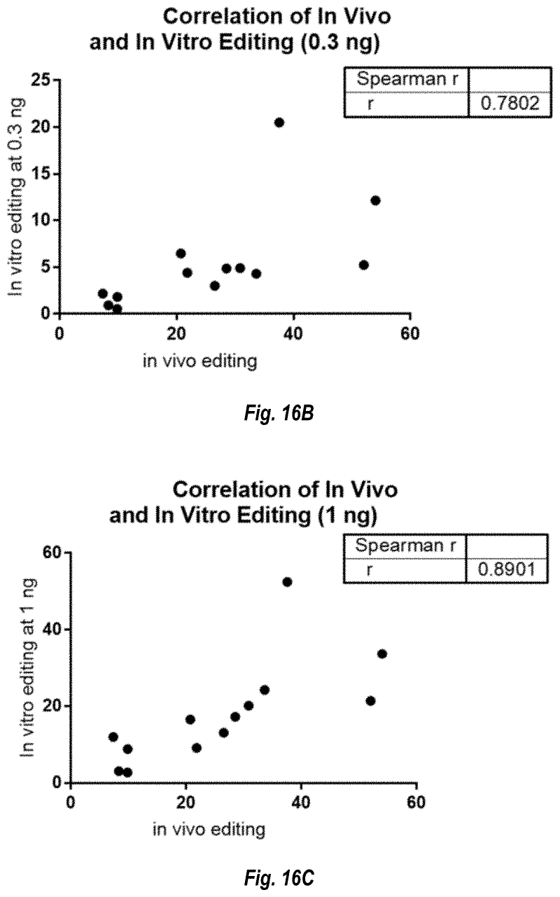

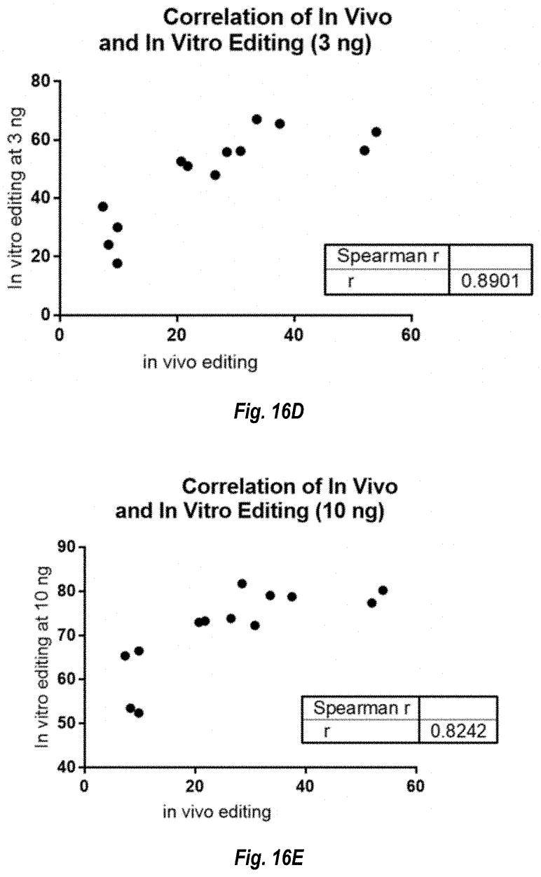

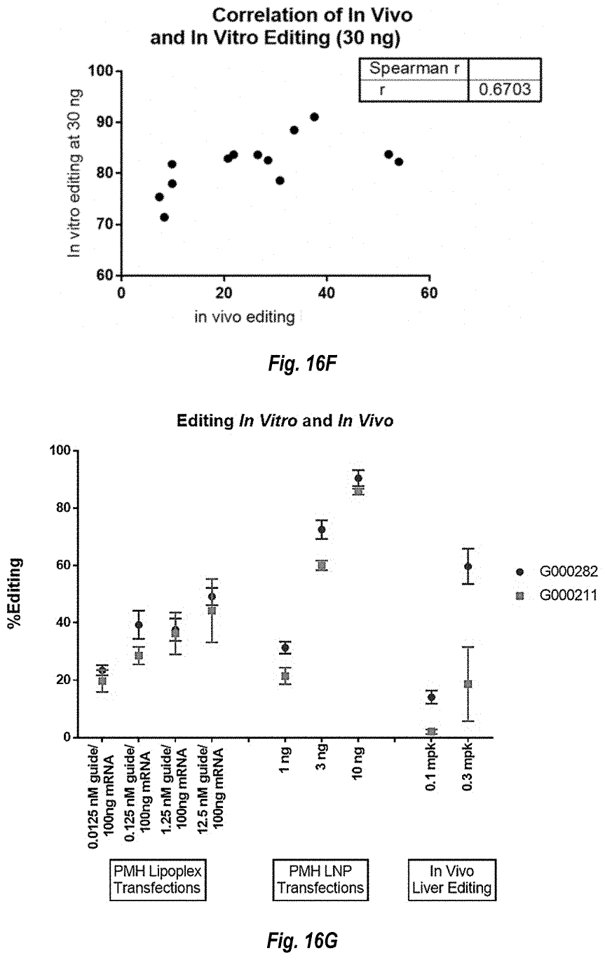

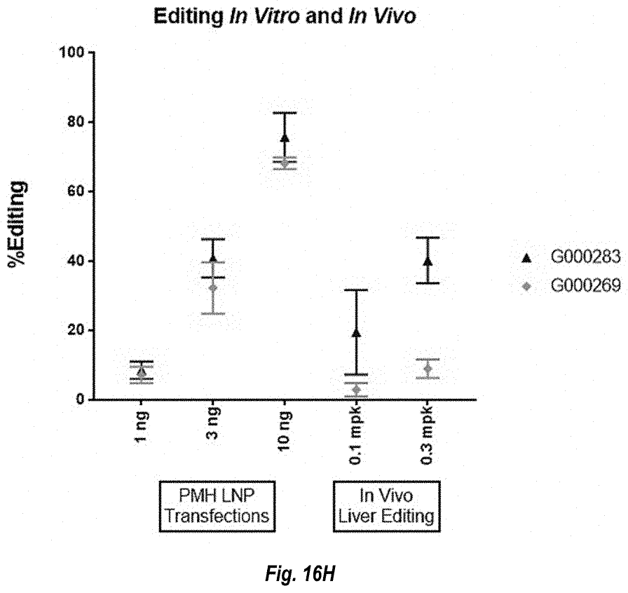

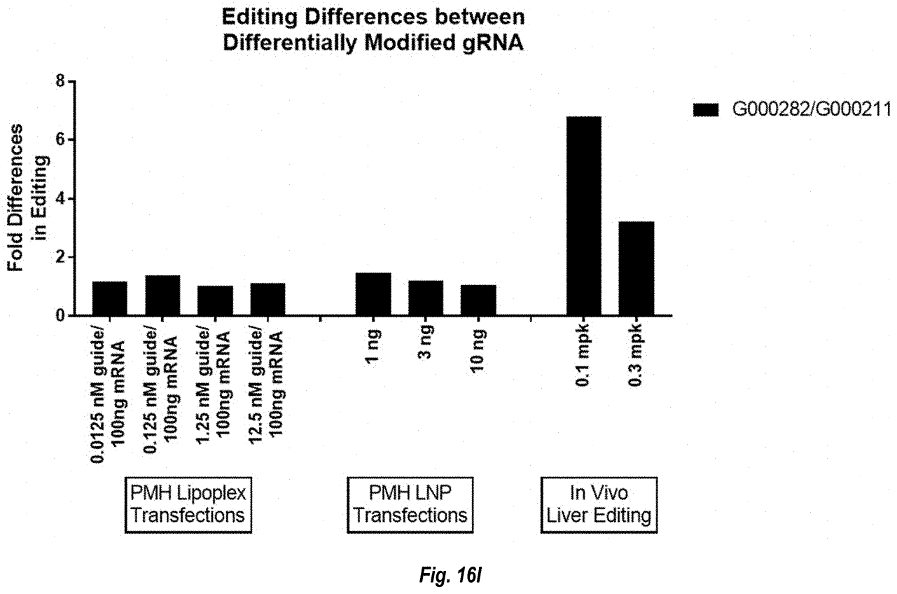

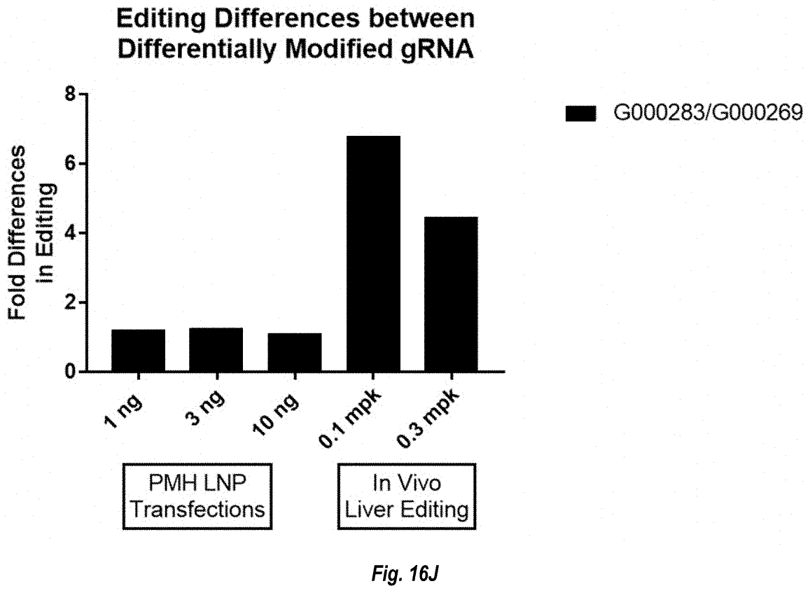

The % editing from in vitro lipoplex transfections of PMH compared to in vivo editing is shown FIG. 16A. As shown in FIG. 16A, the correlation between in vitro lipoplex transfections of PMH and in vivo editing is not statistically significant. The correlation is not predictive.

The % editing from in vitro LNP transfections of PMH (at 0.3 ng, 1 ng, 3 ng, 10 ng, and 30 ng) compared to in vivo editing are shown in FIGS. 16B to 16F. As shown in FIGS. 16B to 16F, the correlation between in vitro LNP transfections of PMH and in vivo editing was statistically significant. The correlation is predictive.

FIG. 16G shows a comparison of % editing with the indicated guides delivered to PMH by lipoplex transfection (data above left box), to PMH in LNP (data above center box), or to mice in vivo (data above right box). FIG. 16H shows a comparison of % editing with the indicated guides delivered to PMH in LNP (1 ng, 3 ng, 10 ng) or to mice in vivo (0.1 mpk, 0.3 mpk). Although the rank order of the indicated guides may generally be considered to be the same in each set of data, the in vivo editing shows greater differentiation of the editing results.

FIG. 16I shows the results from FIG. 16G replotted to indicate differences in editing between G000282 and G000211. The barplot values were generated by dividing % editing of the G000282 value by the % editing of the G000211 value to indicate fold differences in editing. The indicated guides were delivered to PMH by lipoplex transfection (data above left box), to PMH in LNP (data above center box), or to mice in vivo (data above right box).

FIG. 16J shows the results from FIG. 16H replotted to indicate differences in editing between G000283 and G000269. The barplot values were generated by dividing % editing of the G000283 value by the % editing of the G000269 value to indicate fold differences in editing. The indicated guides were delivered to PMH in LNP (data above left box) or to mice in vivo (data above right box).

LENGTHY TABLES
The patent application contains a lengthy table section. A copy of the table is available in electronic form from the USPTO web site (https://seqdata.uspto.gov/?pageRequest=docDetail&DocID=US20210087568A1). An electronic copy of the table will also be available from the USPTO upon request and payment of the fee set forth in 37 CFR 1.19(b)(3).

We claim:

1. A guide RNA (gRNA) which is a short-single guide RNA (short-sgRNA) comprising a conserved portion of an sgRNA comprising a hairpin region, wherein the hairpin region lacks at least 5-10 nucleotides and wherein the short-sgRNA comprises a 5′ end modification or a 3′ end modification.

2. The gRNA of claim 1, wherein the short-sgRNA comprises a 5′ end modification.

3. The gRNA of any one of the preceding claims, wherein the short-sgRNA comprises a 3′ end modification.

4. The gRNA of any one of the preceding claims, wherein the short-sgRNA comprises a 5′ end modification and a 3′ end modification.

5. The gRNA of any one of the preceding claims, wherein the short-sgRNA comprises a 3′ tail.

6. The gRNA of claim 5, wherein the 3′ tail comprises 1, 2, 3, 4, 5, 6, 7, 8, 9, or 10 nucleotides.

7. The gRNA of claim 5, wherein the 3′ tail comprises about 1-2, 1-3, 1-4, 1-5, 1-7, 1-10, at least 1-5, at least 1-3, at least 1-4, at least 1-5, at least 1-5, at least 1-7, or at least 1-10 nucleotides.

8. The gRNA of any one of the preceding claims, wherein the short-sgRNA does not comprise a 3′ tail.

9. The gRNA of any one of the preceding claims, comprising a modification in the hairpin region.

10. The gRNA of any one of the preceding claims, comprising a 3′ end modification, and a modification in the hairpin region.

11. The gRNA of any one of the preceding claims, comprising a 3′ end modification, a modification in the hairpin region, and a 5′ end modification.

12. The gRNA of any one of the preceding claims, comprising a 5′ end modification, and a modification in the hairpin region.

13. The gRNA of any one of the preceding claims, wherein the at least 5-10 lacking nucleotides are consecutive.

14. The gRNA of any one of the preceding claims, wherein the at least 5-10 lacking nucleotides:
i. are within hairpin 1;
ii. are within hairpin 1 and the “N” between hairpin 1 and hairpin 2;
iii. are within hairpin 1 and the two nucleotides immediately 3′ of hairpin 1;
iv. include at least a portion of hairpin 1;
v. are within hairpin 2;
vi. include at least a portion of hairpin 2;
vii. are within hairpin 1 and hairpin 2;
viii. include at least a portion of hairpin 1 and include the “N” between hairpin 1 and hairpin 2;
ix. include at least a portion of hairpin 2 and include the “N” between hairpin 1 and hairpin 2;
x. include at least a portion of hairpin 1, include the “N” between hairpin 1 and hairpin 2, and include at least a portion of hairpin 2;
xi. are within hairpin 1 or hairpin 2, optionally including the “N” between hairpin 1 and hairpin 2;
xii. are consecutive;
xiii. are consecutive and include the “N” between hairpin 1 and hairpin 2;
xiv. are consecutive and span at least a portion of hairpin 1 and a portion of hairpin 2;
xv. are consecutive and span at least a portion of hairpin 1 and the “N” between hairpin 1 and hairpin 2; or
xvi. are consecutive and span at least a portion of hairpin 1 and two nucleotides immediately 3′ of hairpin 1.

15. The gRNA of any one of the preceding claims, further comprising a guide region.

16. The gRNA of any one of the preceding claims, wherein the 3′ and/or 5′ end modification comprises a protective end modification, such as a modified nucleotide selected from 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.

17. The gRNA of any one of the preceding claims, wherein the modification in the hairpin region comprises a modified nucleotide selected from 2′-O-methyl (2′-OMe) modified nucleotide, a 2′-fluoro (2′-F) modified nucleotide, a phosphorothioate (PS) linkage between nucleotides, or combinations thereof.

18. The gRNA of any one of the preceding claims, wherein the 3′ and/or 5′ end modification comprises or further comprises a 2′-O-methyl (2′-OMe) modified nucleotide.

19. The gRNA of any one of the preceding claims, wherein the 3′ and/or 5′ end modification comprises or further comprises a 2′-fluoro (2′-F) modified nucleotide.

20. The gRNA of any one of the preceding claims, wherein the 3′ and/or 5′ end modification comprises or further comprises a phosphorothioate (PS) linkage between nucleotides.

21. The gRNA of any one of the preceding claims, wherein the 3′ and/or 5′ end modification comprises or further comprises an inverted abasic modified nucleotide.

22. The gRNA of any one of the preceding claims, wherein the modification in the hairpin region comprises or further comprises a 2′-O-methyl (2′-OMe) modified nucleotide.

23. The gRNA of any one of the preceding claims, wherein the modification in the hairpin region comprises or further comprises a 2′-fluoro (2′-F) modified nucleotide.

24. The gRNA of any one of the preceding claims, wherein the 3′ end modification comprises any of:
i. a modification of any one or more of the last 7, 6, 5, 4, 3, 2, or 1 nucleotides;
ii. one modified nucleotide;
iii. two modified nucleotides;
iv. three modified nucleotides;
v. four modified nucleotides;
vi. five modified nucleotides;
vii. six modified nucleotides; and
viii. seven modified nucleotides.

25. The gRNA of any one of the preceding claims, wherein the at least 5-10 nucleotides comprise nucleotides 54-61 of SEQ ID NO:400, nucleotides 53-60 of SEQ ID NO:400; or nucleotides 54-58 of SEQ ID NO:400, optionally wherein the short-sgRNA comprises modifications at least H1-1 to H1-5 and H2-1 to H2-12.

26. The gRNA of any one of the preceding claims, wherein the at least 5-10 nucleotides:
i. consist of 5-10 nucleotides;
ii. consist of 6-10 nucleotides;
iii. consist of 5 nucleotides;
iv. consist of 6 nucleotides;
v. consist of 7 nucleotides;
vi. consist of 8 nucleotides;
vii. consist of 9 nucleotides;
viii. consist of 10 nucleotides;
ix. consist of 5-10 consecutive nucleotides;
x. consist of 6-10 consecutive nucleotides;
xi. consist of 5 consecutive nucleotides;
xii. consist of 6 consecutive nucleotides;
xiii. consist of 7 consecutive nucleotides;
xiv. consist of 8 consecutive nucleotides;
xv. consist of 9 consecutive nucleotides; or
xvi. consist of 10 consecutive nucleotides.

27. The gRNA of any one of the preceding claims, wherein the 3′ end modification comprises one or more of:
i. a phosphorothioate (PS) linkage between nucleotides;
ii. a 2′-OMe modified nucleotide;
iii. a 2′-O-moe modified nucleotide;
iv. a 2′-F modified nucleotide;
v. an inverted abasic modified nucleotide; and
vi. a combination of one or more of (i.)-(v.).

28. The gRNA of any one of the preceding claims, wherein the short-sgRNA comprises a 3′ tail comprising one or more of:
i. a phosphorothioate (PS) linkage between nucleotides;
ii. a 2′-OMe modified nucleotide;
iii. a 2′-O-moe modified nucleotide;
iv. a 2′-F modified nucleotide;
v. an inverted abasic modified nucleotide; and
vi. a combination of one or more of (i.)-(v.).

29. The gRNA any one of the preceding claims, wherein the short-sgRNA comprises one or more of:
i. 1, 2, 3, 4, 5, 6, 7, 8, 9, or 10 PS linkages between nucleotides;
ii. 1, 2, 3, 4, 5, 6, 7, 8, 9, 10, 12, 14, 16, or 18 PS linkages between nucleotides;
iii. about 1-3, 1-5, 1-6, 1-7, 1-8, 1-9, or 1-10 PS linkages between nucleotides;
iv. about 1-3, 1-5, 1-6, 1-7, 1-8, 1-9, 1-10, 1-12, 1-14, 1-16, 1-18, or 1-20 PS linkages between nucleotides; and
v. PS linkages between each nucleotide.

30. The gRNA of any one of the preceding claims, wherein the 3′ end modification comprises at least one PS linkage, and wherein one or more of:
i. there is one PS linkage, and the linkage is between the last and second to last nucleotide;
ii. there are two PS linkages between the last three nucleotides;
iii. there are PS linkages between any one or more of the last four nucleotides;
iv. there are PS linkages between any one or more of the last five nucleotides; and
v. there are PS linkages between any one or more of the last 2, 3, 4, 5, 6, 7, 8, 9, or 10 nucleotides.

31. The gRNA of claim 30, wherein the 3′ end modification further comprises at least one 2′-OMe, 2′-O-moe, inverted abasic, or 2′-F modified nucleotide.

32. The gRNA of any one of the preceding claims, wherein the 3′ end modification comprises:
i. a modification of one or more of the last 1-7 nucleotides, wherein the modification is a PS linkage, inverted abasic nucleotide, 2′-OMe, 2′-O-moe, 2′-F, or combinations thereof;
ii. a modification to the last nucleotide with 2′-OMe, 2′-O-moe, 2′-F, or combinations thereof, and an optional one or two PS linkages to the next nucleotide and/or the first nucleotide of the 3′ tail;
iii. a modification to the last and/or second to last nucleotide with 2′-OMe, 2′-O-moe, 2′-F, or combinations thereof, and optionally one or more PS linkages;
iv. a modification to the last, second to last, and/or third to last nucleotides with 2′-OMe, 2′-O-moe, 2′-F, or combinations thereof, and optionally one or more PS linkages;
v. a modification to the last, second to last, third to last, and/or fourth to last nucleotides with 2′-OMe, 2′-O-moe, 2′-F, or combinations thereof, and optionally one or more PS linkages; or
vi. a modification to the last, second to last, third to last, fourth to last, and/or fifth to last nucleotides with 2′-OMe, 2′-O-moe, 2′-F, or combinations thereof, and optionally one or more PS linkages.

33. The gRNA of any one of the preceding claims, wherein the sgRNA comprise a 3′ tail, wherein the 3′ tail comprises a modification of any one or more of the nucleotides present in the 3′ tail.

34. The gRNA of claim 33, wherein the 3′ tail is fully modified.

35. The gRNA of claim 33, wherein the at least 5-10 nucleotides comprise nucleotides 54-61 of SEQ ID NO:400, nucleotides 53-60 of SEQ ID NO:400; or nucleotides 54-58 of SEQ ID NO:400, optionally wherein the short-sgRNA comprises modifications at least H1-1 to H1-5 and H2-1 to H2-12.

36. The gRNA of any one of the preceding claims, wherein the short-sgRNA comprises any one or more of:
i. the 3′ end modification as shown in any one of SEQ ID Nos: 1-54;
ii. (i) a 2′-OMe modified nucleotide at the last nucleotide of the conserved region of an sgRNA or short-sgRNA, (ii) three consecutive 2′O-moe modified nucleotides immediately 5′ to the 2′-OMe modified nucleotide, and (iii) three consecutive PS linkages between the last three nucleotides;
iii. (i) five consecutive 2′-OMe modified nucleotides from the 3′ end of the 3′ terminus, and (ii) three PS linkages between the last three nucleotides;
iv. an inverted abasic modified nucleotide at the last nucleotide of the conserved region of an sgRNA or short-sgRNA;
v. (i) an inverted abasic modified nucleotide at the last nucleotide of the conserved region of an sgRNA or short-sgRNA, and (ii) three consecutive 2′-OMe modified nucleotides at the last three nucleotides of the conserved region of an sgRNA or short-sgRNA;
vi. (i) 15 consecutive 2′-OMe modified nucleotides from the 3′ end of the 3′ terminus, (ii) five consecutive 2′-F modified nucleotides immediately 5′ to the 2′-OMe modified nucleotides, and (iii) three PS linkages between the last three nucleotides;
vii. (i) alternating 2′-OMe modified nucleotides and 2′-F modified nucleotides at the last 20 nucleotides of the conserved region of an sgRNA or short-sgRNA, and (ii) three PS linkages between the last three nucleotides;
viii. (i) two or three consecutive 2′-OMe modified nucleotides, and (ii) three PS linkages between the last three nucleotides;
ix. one PS linkage between the last and next to last nucleotides; and
x. 15 or 20 consecutive 2′-OMe modified nucleotides, and (ii) three PS linkages between the last three nucleotides.

37. The gRNA of any one of the preceding claims, wherein the 5′ end modification comprises any one or more of:
i. a modification of any one or more of nucleotides 1-7 of the guide region;
ii. one modified nucleotide;
iii. two modified nucleotides;
iv. three modified nucleotides;
v. four modified nucleotides;
vi. five modified nucleotides;
vii. six modified nucleotides; and
viii. seven modified nucleotides.

38. The gRNA of any one of the preceding claims, wherein the 5′ end modification comprises a modification of between 1 and 7, between 1 and 5, between 1 and 4, between 1 and 3, or between 1 and 2 nucleotides.

39. The gRNA of any one of the preceding claims, wherein the at least 5-10 nucleotides:
i. comprise nucleotides 54-61 of SEQ ID NO:400;
ii. comprise nucleotides 53-60 of SEQ ID NO:400;
iii. comprise nucleotides 54-58 of SEQ ID NO:400.
iv. consist of nucleotides 54-61 of SEQ ID NO:400;
v. consist of nucleotides 53-60 of SEQ ID NO:400; or
vi. consist of nucleotides 54-58 of SEQ ID NO:400.

40. The gRNA of any one of the preceding claims, wherein the 5′ end modification comprises one or more of:
i. a phosphorothioate (PS) linkage between nucleotides;
ii. a 2′-OMe modified nucleotide;
iii. a 2′-O-moe modified nucleotide;
iv. a 2′-F modified nucleotide;
v. an inverted abasic modified nucleotide;
vi. a deoxyribonucleotide;
vii. an inosine; and
viii. combinations of one or more of (i.)-(vii.).

41. The gRNA any one of the preceding claims, wherein the 5′ end modification comprises:
i. 1, 2, 3, 4, 5, 6, and/or 7 PS linkages between nucleotides; or
ii. about 1-2, 1-3, 1-4, 1-5, 1-6, or 1-7 PS linkages between nucleotides.

42. The gRNA of any one of the preceding claims, wherein the 5′ end modification comprises at least one PS linkage, and wherein:
i. there is one PS linkage, and the linkage is between nucleotides 1 and 2 of the guide region;
ii. there are two PS linkages, and the linkages are between nucleotides 1 and 2, and 2 and 3 of the guide region;
iii. there are PS linkages between any one or more of nucleotides 1 and 2, 2 and 3, and 3 and 4 of the guide region;
iv. there are PS linkages between any one or more of nucleotides 1 and 2, 2 and 3, 3 and 4, and 4 and 5 of the guide region;
v. there are PS linkages between any one or more of nucleotides 1 and 2, 2 and 3, 3 and 4, 4 and 5, and 5 and 6 of the guide region;
vi. there are PS linkages between any one or more of nucleotides 1 and 2, 2 and 3, 3 and 4, 4 and 5, 5 and 6, and 6 and 7 of the guide region; or
vii. there are PS linkages between any one or more of nucleotides 1 and 2, 2 and 3, 3 and 4, 4 and 5, 5 and 6, 6 and 7, and 7 and 8 of the guide region.

43. The gRNA of claim 42, wherein the 5′ end modification further comprises at least one 2′-OMe, 2′-O-moe, inverted abasic, or 2′-F modified nucleotide.

44. The gRNA of any one of the preceding claims, wherein the short-sgRNA comprises:
i. a modification of one or more of nucleotides 1-7 of the variable region, wherein the modification is a PS linkage, inverted abasic nucleotide, 2′-OMe, 2′-O-moe, 2′-F, 2′-H (a deoxyribonucleotide), an inosine, and/or combinations thereof;
ii. a modification to the first nucleotide of the guide region with 2′-OMe, 2′-O-moe, 2′-F, 2′-H, an inosine, or combinations thereof, and an optional PS linkage to the next nucleotide;
iii. a modification to the first and/or second nucleotide of the variable region with 2′-OMe, 2′-O-moe, 2′-F, 2′-H, an inosine, or combinations thereof, and optionally one or more PS linkages;
iv. a modification to the first, second, and/or third nucleotides of the variable region with 2′-OMe, 2′-O-moe, 2′-F, 2′-H, an inosine, or combinations thereof, and optionally one or more PS linkages;
v. a modification to the first, second, third, and/or fourth nucleotides of the variable region with 2′-OMe, 2′-O-moe, 2′-F, 2′-H, an inosine, or combinations thereof, and optionally one or more PS linkages; or
vi. a modification to the first, second, third, fourth, and/or fifth nucleotides of the variable region with 2′-OMe, 2′-O-moe, 2′-F, 2′-H, an inosine, or combinations thereof, and optionally one or more PS linkages.

45. The gRNA of any one of the preceding claims, wherein the short-sgRNA comprises any one or more of:
i. a 5′ end modification as shown in any one of SEQ ID Nos: 1-54;
ii. 2′-OMe modified nucleotides at nucleotides 1, 2, and 3 of the guide region;
iii. 2′-OMe modified nucleotides at nucleotides 1, 2, and 3 of the guide region and PS linkages between nucleotides 1 and 2, 2 and 3, and 3 and 4 of the guide region;
iv. 2′-OMe modified nucleotides at nucleotides 1, 2, 3, 4, and 5 of the guide region;
v. 2′-OMe modified nucleotides at nucleotides 1, 2, 3, 4, and 5 of the guide region and PS linkages between nucleotides 1 and 2, 2 and 3, 3 and 4, 4 and 5, and 5 and 6 of the guide region;
vi. 2′O-moe modified nucleotides at nucleotides 1, 2, and 3 of the guide region;
vii. 2′O-moe modified nucleotides at nucleotides 1, 2, and 3 of the guide region and PS linkages between nucleotides 1 and 2, 2 and 3, and 3 and 4 of the guide region;
viii. an inverted abasic modified nucleotide at nucleotide 1 of the guide region;
ix. an inverted abasic modified nucleotide at nucleotide 1 of the guide region and 2′-OMe modified nucleotides at nucleotides 1, 2, and 3 of the guide region; and
x. an inverted abasic modified nucleotide at nucleotide 1 of the guide region, 2′-OMe modified nucleotides at nucleotides 1, 2, and 3 of the guide region, and PS linkages between nucleotides 1 and 2, 2 and 3, 3 and 4, 4 and 5, and 5 and 6 of the variable region.

46. The gRNA of any one of the preceding claims, wherein the upper stem region comprises at least one modification.

47. The gRNA of any one of the preceding claims, wherein the upper stem modification comprises any one or more of:
i. a modification to any one or more of US1-US12 in the upper stem region;
ii. a modification of at least 1, 2, 3, 4, 5, 6, 7, 8, 9, 10, 11, or all 12 nucleotides in the upper stem region; and
iii. a modification of about 1-2, 1-3, 1-4, 1-5, 1-6, 1-7, 1-8, 1-9, 1-10, or 1-12 nucleotides in the upper stem region.

48. The gRNA of claim 47, wherein the upper stem modification comprises one or more of:
i. a 2′-OMe modified nucleotide;
ii. a 2′-O-moe modified nucleotide;
iii. a 2′-F modified nucleotide; and
iv. combinations of one or more of (i.)-(iii.).

49. A guide RNA which is a short-sgRNA comprising a conserved portion of an sgRNA comprising a hairpin region, wherein the hairpin region lacks at least 5-10 nucleotides and wherein the short-sgRNA comprises a 5′ end modification and one or more modification in one or more of:
i. the upper stem region;
ii. the hairpin 1 region; and
iii. the hairpin 2 region,
wherein the 5′ end modification comprises a 5′ protective end modification, such as at least two phosphorothioate (PS) linkages within the first seven nucleotides.

50. The gRNA of claim 49, wherein at least one modification comprises a 2′-O-methyl (2′-OMe) modified nucleotide.

51. The gRNA of claim 49 or claim 50, wherein at least one modification comprises a 2′-fluoro (2′-F) modified nucleotide.

52. The gRNA of any one of claims 49-51, wherein at least one modification comprises a phosphorothioate (PS) bond between nucleotides.

53. The gRNA of any one of claims 49-52, wherein the short-sgRNA comprises one or more modifications in the upper stem region.

54. The gRNA of claim 53, comprising modifications at any one of US1 to US12.

55. The gRNA of any one of claims 49-54, wherein the short-sgRNA comprises one or more modifications in the hairpin 1 region.

56. The gRNA of claim 55, wherein the short-sgRNA comprises a modification at H1-1.

57. The gRNA of any one of claims 49-56, wherein the short-sgRNA comprises one or more modifications in the hairpin 2 region.

58. The gRNA of claim 57, wherein the short-sgRNA comprises a modification at H2-1.

59. The gRNA of any one of claims 49-58, wherein the short-sgRNA comprises modifications at H1-1 to H1-12.

60. The gRNA of any one of claims 49-59, wherein the short-sgRNA comprises modifications at H2-1 to H2-15.

61. The gRNA of any one of claims 49-60, wherein the short-sgRNA comprises one or more modifications in each of the upper stem region, the hairpin 1 region, and the hairpin 2 region.

62. The gRNA of any one of claims 49-61, wherein the short-sgRNA comprises a modified nucleotide between hairpin 1 and hairpin 2 regions.

63. The gRNA of any one of claims 49-62, further comprising a lower stem region comprising a modification.

64. The gRNA of any one of claims 49-63, further comprising a 3′ end modification.

65. The gRNA of claim 64, wherein at least two of the last four nucleotides at the 3′ end of the 3′ terminus are modified.

66. The gRNA of claim 64, wherein at least two of the last four nucleotides at the 3′ end of the 3′ terminus are modified with 2′-OMe, 2′-F, or 2′-O-moe.

67. The gRNA of any one of claims 64-66, further comprising phosphorothioate (PS) bonds between one or more of the last four nucleotides at the 3′ end of the 3′ terminus.

68. The gRNA of any one of claims 49-67, further comprising a bulge region comprising a modification.

69. The gRNA of any one of claims 49-68, further comprising a nexus region comprising a modification.

70. The gRNA of any one of claims 49-69, wherein at least the first three nucleotides at the 5′ end of the variable region, and the last three nucleotides at the 3′ end of the 3′ terminus are modified.

71. The gRNA of any one of claims 49-70, wherein the first four nucleotides at the 5′ end of the variable region, and the last four nucleotides at the 3′ end of the 3′ terminus are linked with phosphorothioate (PS) bonds.

72. The gRNA of any one of claims 70-71, wherein the end modifications comprise 2′-OMe.

73. The gRNA of any one of claims 70-71, wherein the end modifications comprise 2′-F.

74. The gRNA of any one of claims 49-73, wherein the first four nucleotides at the 5′ end of the variable region and the last four nucleotides at the 3′ end of the 3′ terminus are linked with a PS bond, and wherein the first three nucleotides at the 5′ end of the variable region and the last three nucleotides at the 3′ end of the 3′ terminus comprise 2′-OMe modifications.

75. The gRNA of any one of claims 49-74, wherein the first four nucleotides at the 5′ terminus and the last four nucleotides at the 3′ terminus are linked with a PS bond, and wherein the first three nucleotides at the 5′ terminus and the last three nucleotides at the 3′ terminus comprise 2′-OMe, 2′-F, and/or 2′-O-moe modifications.

76. The gRNA of any one of claims 49-75, wherein LS1, LS6, LS7, LS8, LS11, and/or LS12 are modified with 2′-OMe.

77. The gRNA of any one of claims 49-76, wherein each of the nucleotides in the bulge region are modified with 2′-OMe.

78. The gRNA of any one of claims 49-77, wherein at least 50% of the nucleotides in the bulge region are modified with 2′-OMe.

79. The gRNA of any one of claims 49-78, wherein each of the nucleotides in the upper stem region are modified with 2′-OMe.

80. The gRNA of any one of claims 49-79, wherein N16, N17, and/or N18 in the nexus region are modified with 2′-OMe.

81. The gRNA of any one of claims 49-80, wherein N15, N16, N17, and/or N18 in the nexus region are modified.

82. The gRNA of claim 80 or 81, wherein the modifications in the nexus region are selected from 2′-OMe and 2′F.

83. The gRNA of any one of claims 80-82, wherein N16, N17, and N18 are linked with PS bonds.

84. The gRNA of any one of claims 49-83, wherein each of the nucleotides remaining in the hairpin 1 region are modified with 2′-OMe.

85. The gRNA of any one of claims 49-84, wherein each of the nucleotides in the hairpin 2 region are modified with 2′-OMe.

86. A guide RNA which is a short-sgRNA comprising a conserved portion of an sgRNA comprising a hairpin region, wherein the hairpin region lacks at least 5-10 nucleotides and wherein the short-sgRNA comprises a 5′ end modification and a 3′ end modification, wherein the short-sgRNA further comprises any one or more of:
i. at least one modification in the upper stem region; and
ii. a 3′ tail.

87. The gRNA of claim 86, wherein the upper stem modification comprises any one or more of:
i. a modification of each nucleotide (US1-US12) in the upper stem region;
ii. a modification to any one or more of US1-US12 in the upper stem region;
iii. a modification of at least 1, 2, 3, 4, 5, 6, 7, 8, 9, 10, 11, or all 12 nucleotides in the upper stem region; and
iv. a modification of about 1-2, 1-3, 1-4, 1-5, 1-6, 1-7, 1-8, 1-9, 1-10, or 1-12 nucleotides in the upper stem region.

88. The gRNA of any one of claims 86-87, wherein the 5′ end modification comprises any one or more of:
i. a modification of any one or more of nucleotides 1-7 of the variable region;
ii. one modified nucleotide;
iii. two modified nucleotides;
iv. three modified nucleotides;
v. four modified nucleotides;
vi. five modified nucleotides;
vii. six modified nucleotides; and
viii. seven modified nucleotides.

89. The gRNA of any one of claims 86-88, wherein the 5′ end modification comprises any one or more of:
i. a 5′ end modification as shown in any one of SEQ ID Nos: 1-54, 401-532, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, 3388-3430, or 3549-3552;
ii. 2′-OMe modified nucleotides at nucleotides 1, 2, and 3 of the variable region;
iii. 2′-OMe modified nucleotides at nucleotides 1, 2, and 3 of the variable region and PS linkages between nucleotides 1 and 2, 2 and 3, and 3 and 4 of the variable region;
iv. 2′-OMe modified nucleotides at nucleotides 1, 2, 3, 4, and 5 of the variable region;
v. 2′-OMe modified nucleotides at nucleotides 1, 2, 3, 4, and 5 of the variable region and PS linkages between nucleotides 1 and 2, 2 and 3, 3 and 4, 4 and 5, and 5 and 6 of the variable region;
vi. 2′O-moe modified nucleotides at nucleotides 1, 2, and 3 of the variable region;
vii. 2′O-moe modified nucleotides at nucleotides 1, 2, and 3 of the variable region and PS linkages between nucleotides 1 and 2, 2 and 3, and 3 and 4 of the variable region;
viii. an inverted abasic modified nucleotide at nucleotide 1 of the variable region;
ix. an inverted abasic modified nucleotide at nucleotide 1 of the variable region and 2′-OMe modified nucleotides at nucleotides 1, 2, and 3 of the variable region; and
x. an inverted abasic modified nucleotide at nucleotide 1 of the variable region, 2′-OMe modified nucleotides at nucleotides 1, 2, and 3 of the variable region, and PS linkages between nucleotides 1 and 2, 2 and 3, 3 and 4, 4 and 5, and 5 and 6 of the variable region.

90. The gRNA of claim 89, comprising 2′-OMe modified nucleotides at at least nucleotides 1, 2, and 3, of the variable region and PS linkages between nucleotides 1 and 2, 2 and 3, and 3 and 4 of the variable region.

91. The gRNA of claim 89, comprising 2′-OMe modified nucleotides at at least nucleotides 1, 2, 3, and 4 of the variable region and PS linkages between nucleotides 1 and 2, 2 and 3, and 3 and 4 of the variable region.

92. The gRNA of any one of claims 86-91, comprising a 3′ end modification comprising any one or more of:
i. a modification of any one or more of the last 7, 6, 5, 4, 3, 2, or 1 nucleotides;
ii. one modified nucleotide;
iii. two modified nucleotides;
iv. three modified nucleotides;
v. four modified nucleotides;
vi. five modified nucleotides;
vii. six modified nucleotides; and
viii. seven modified nucleotides.

93. The gRNA of any one of claims 86-92, wherein the short-sgRNA comprises any one or more of:
i. a 3′ end modification shown in any one of SEQ ID Nos: 1-54, 401-532, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, 3388-3430, or 3549-3552;
ii. (i) a 2′-OMe modified nucleotide at the last nucleotide of the conserved region of an sgRNA or short-sgRNA, (ii) three consecutive 2′O-moe modified nucleotides immediately 5′ to the 2′-OMe modified nucleotide, and (iii) three consecutive PS linkages between the last three nucleotides;
iii. (i) five consecutive 2′-OMe modified nucleotides, and (ii) three PS linkages between the last three nucleotides;
iv. an inverted abasic modified nucleotide at the last nucleotide of the conserved region of an sgRNA or short-sgRNA;
v. (i) an inverted abasic modified nucleotide at the last nucleotide of the conserved region of an sgRNA or short-sgRNA, and (ii) three consecutive 2′-OMe modified nucleotides at the last three nucleotides of the conserved region of an sgRNA or short-sgRNA;
vi. (i) 15 consecutive 2′-OMe modified nucleotides, (ii) five consecutive 2′-F modified nucleotides immediately 5′ to the 2′-OMe modified nucleotides, and (iii) three PS linkages between the last three nucleotides;
vii. (i) alternating 2′-OMe modified nucleotides and 2′-F modified nucleotides at the last 20 nucleotides of the conserved region of an sgRNA or short-sgRNA, and (ii) three PS linkages between the last three nucleotides;
viii. (i) two or three consecutive 2′-OMe modified nucleotides, and (ii) three PS linkages between the last three nucleotides;
ix. one PS linkage between the last and next to last nucleotides; and
x. 15 or 20 consecutive 2′-OMe modified nucleotides, and (ii) three PS linkages between the last three nucleotides.

94. The gRNA of any one of claims 86-93, wherein the sgRNA comprise a 3′ tail, wherein the 3′ tail comprises a modification of any one or more of the nucleotides present in the 3′ tail.

95. The gRNA of claim 94, wherein the 3′ tail is fully modified.

96. The gRNA of claim 94 wherein the 3′ tail comprises 1, 2, 3, 4, 5, 6, 7, 8, 9, 10, 1-2, 1-3, 1-4, 1-5, 1-6, 1-7, 1-8, 1-9, or 1-10 nucleotides, optionally where any one or more of these nucleotides are modified.

97. A guide RNA which is a short-sgRNA comprising any of SEQ ID Nos: 1-54, 201-254, and 301-354, including the modifications of Table 1.

98. A guide RNA which is a short-sgRNA comprising nucleic acids having at least 99, 98, 97, 96, 95, 94, 93, 92, 91, 90, 85, 80, 75, or 70% identity to the nucleic acids of any one of SEQ ID Nos: 1-54, 201-254, and 301-354, wherein the modification at each nucleotide of the short-sgRNA that corresponds to a nucleotide of the reference sequence identifier in Table 1, is identical to or equivalent to the modification shown in the reference sequence identifier in Table 1.

99. The gRNA of any one of the preceding claims, comprising a YA modification at at least one guide region YA site.

100. The gRNA of any one of the preceding claims, comprising a YA modification at at least one guide region YA site that is not a 5′ end modification.

101. The gRNA of any one of the preceding claims, comprising a YA modification at one or more guide region YA sites, wherein the guide region YA site is at or after nucleotide 8 from the 5′ end of the 5′ terminus.

102. The gRNA of any one of the preceding claims comprising a YA modification at one or more guide region YA sites, wherein the short-sgRNA comprises one or more of:
i. a modification at one or more of H1-1 and H2-1;
ii. a YA modification at 1, 2, 3, 4, or 5 guide region YA sites;
iii. a YA modification at 1, 2, 3, 4, or 5 guide region YA sites, wherein the modification of at least one guide region YA site is different from any 5′ end modification of the sgRNA;
iv. a YA modification at one or more guide region YA sites, wherein the guide region YA site is at or after nucleotide 8 from the 5′ end of the 5′ terminus;
v. a YA modification at one or more guide region YA sites, wherein the guide region YA site is within nucleotides 5-end, 6-end, 7-end, 8-end, 9-end, or 10-end from the 5′ end of the 5′ terminus;
vi. a YA modification at one or more guide region YA sites, wherein the guide region YA site is within 17, 16, 15, 14, 13, 12, 11, 10, or 9 nucleotides of the 3′ terminal nucleotide of the guide region;
vii. a YA modification at a guide region YA site other than a 5′ end modification;
viii. a YA modification at two or more guide region YA sites, wherein the guide region YA sites are at or after nucleotide 8 from the 5′ end of the 5′ terminus;
ix. a YA modification at two or more guide region YA sites, wherein the two guide region YA sites are within nucleotides 5-end, 6-end, 7-end, 8-end, 9-end, or 10-end from the 5′ end of the 5′ terminus;
x. a YA modification at two or more guide region YA sites, wherein the guide region YA sites are within 17, 16, 15, 14, 13, 12, 11, 10, or 9 nucleotides of the 3′ terminal nucleotide of the guide region;
xi. a YA modification at two or more guide region YA sites other than a 5′ end modification; and
xii. a YA modification at two or more guide region YA sites, wherein the modifications of the guide region YA sites comprise a modification that at least one nucleotide located 5′ of the guide region YA site does not comprise.

103. The gRNA of any one of the preceding claims, comprising a YA modification wherein the modification comprises 2′-fluoro, 2′-H, 2′-OMe, ENA, UNA, inosine, or PS.

104. The gRNA of any one of the preceding claims, comprising a YA modification wherein the modification alters the structure of the dinucleotide motif to reduce RNA endonuclease activity.

105. The gRNA of any one of the preceding claims, comprising a YA modification wherein the modification interferes with recognition or cleavage of a YA site by an RNase and/or stabilizes an RNA structure.

106. The gRNA of any one of the preceding claims, comprising a YA modification wherein the modification comprises one or more of:
i. a ribose modification selected from 2′-O-alkyl, 2′-F, 2′-moe, 2′-F arabinose, and 2′-H (deoxyribose);
ii. a bicyclic ribose analog, such as LNA, BNA, and ENA;
iii. an unlocked nucleic acid modification;
iv. a base modification, such as inosine, pseudouridine, and 5′-methylcytosine; and
v. an internucleoside linkage modification such as phosphorothioate.

107. The gRNA of any one of the preceding claims, comprising a YA modification at one or more conserved region YA sites.

108. The gRNA of any one of the preceding claims, comprising a YA modification at one or more of conserved region YA sites 2, 3, 4, and 10.

109. The gRNA of any one of the preceding claims, comprising a YA modification at one or more of conserved region YA sites 1 and 8.

110. The gRNA of any one of the preceding claims, comprising a YA modification of conserved region YA site 1.

111. The gRNA of any one of the preceding claims, comprising a YA modification of conserved region YA site 2.

112. The gRNA of any one of the preceding claims, comprising a YA modification of conserved region YA site 3.

113. The gRNA of any one of the preceding claims, comprising a YA modification of conserved region YA site 4.

114. The gRNA of any one of the preceding claims, comprising a YA modification of conserved region YA site 5.

115. The gRNA of any one of the preceding claims, comprising a YA modification of conserved region YA site 6.

116. The gRNA of any one of the preceding claims, comprising a YA modification of conserved region YA site 7.

117. The gRNA of any one of the preceding claims, comprising a YA modification of conserved region YA site 8.

118. The gRNA of any one of the preceding claims, comprising a YA modification of conserved region YA site 9.

119. The gRNA of any one of the preceding claims, comprising a YA modification of conserved region YA site 10.

120. The gRNA of any one of the preceding claims, comprising one or more of:
i. YA modifications of conserved region YA sites 2, 3, 4, and 10;
ii. YA modifications of conserved region YA sites 2, 3, and 4;
iii. YA modifications of conserved region YA sites 2, 3, and 10;
iv. YA modifications of conserved region YA sites 2, 4, and 10;
v. YA modifications of conserved region YA sites 3, 4, and 10;
vi. YA modifications of conserved region YA sites 2 and 10;
vii. YA modifications of conserved region YA sites 2 and 4;
viii. YA modifications of conserved region YA sites 2 and 3;
ix. YA modifications of conserved region YA sites 3 and 4;
x. YA modifications of conserved region YA sites 3 and 10;
xi. YA modifications of conserved region YA sites 4 and 10
xii. YA modifications of conserved region YA sites 1 and 5;
xiii. YA modifications of conserved region YA sites 1 and 6;
xiv. YA modifications of conserved region YA sites 1 and 7;
xv. YA modifications of conserved region YA sites 1 and 8;
xvi. YA modifications of conserved region YA sites 1 and 9;
xvii. YA modifications of conserved region YA sites 8 and 5;
xviii. YA modifications of conserved region YA sites 8 and 6;
xix. YA modifications of conserved region YA sites 8 and 7; and
xx. YA modifications of conserved region YA sites 8 and 9;
xxi. optionally wherein the sgRNA further comprises YA modifications of conserved region YA sites 2, 3, 4, and/or 10.

121. The gRNA of any one of the preceding claims, wherein at least one modified YA site comprises a 2′-OMe modification, optionally at the pyrimidine of the YA site.

122. The gRNA of any one of the preceding claims, wherein at least one modified YA site comprises a 2′-fluoro modification, optionally at the pyrimidine of the YA site.

123. The gRNA of any one of the preceding claims, wherein at least one modified YA site comprises a PS modification, optionally at the pyrimidine of the YA site.

124. The gRNA of any one of the preceding claims, wherein the gRNA comprises a guide region that comprises modifications at at least 1, 2, 3, 4, 5, 6, 7, 8, 9, 10, 11, 12, 13, or all of the following nucleotides: 1, 2, 3, 4, 6, 7, 8, 9, 10, 11, 13, 14, 17, and 18, optionally wherein the modifications are 2′-OMe, 2′-fluoro, 2′-H, inosine, or phosphorothioate modifications.

125. The gRNA of any one of the preceding claims, wherein the short-sgRNA comprises a guide region that comprises modifications at nucleotides 1, 2, 3, 4, 6, 7, 8, 9, 10, 11, 13, 14, 17, and 18, optionally wherein the modifications are 2′-OMe, 2′-fluoro, 2′-H, inosine, or phosphorothioate modifications.

126. The gRNA of claims 124-125, wherein 2′-OMe modifications are not present in the guide region at nucleotides 6-11 and 13-end.

127. The gRNA of claims 124-126, wherein 2′-fluoro modifications are not present in the guide region at nucleotides 1-7, 15, 16, and 19-end.

128. The gRNA of claims 124-127, wherein phosphorothioate modifications are not present in the guide region at nucleotides 4, 5, 11-14, 17, and 18.

129. The gRNA of claims 124-128, wherein the guide region comprises an unmodified nucleotide 20.

130. The gRNA of claims 124-129, wherein the guide region consists of 20 nucleotides.

131. The gRNA of claims 124-130, wherein the guide region comprises a YA site at nucleotides 5-6 and a modification at nucleotide 5.

132. The gRNA of claims 124-131, wherein the guide region comprises a YA site at nucleotides 12-13 and a modification at nucleotide 12.

133. The gRNA of claims 124-132, wherein the guide region comprises a YA site at nucleotides 15-16 and a modification at nucleotide 15.

134. The gRNA of claims 124-133, wherein the guide region comprises a YA site at nucleotides 16-17 and a modification at nucleotide 16.

135. The gRNA of claims 124-134, wherein the guide region comprises a YA site at nucleotides 19-20 and a modification at nucleotide 19.

136. The gRNA of claim 124-130 or 132-135, wherein the guide region does not comprise a YA site at nucleotides 5-6 and nucleotide 5 is unmodified.

137. The gRNA of claim 124-131 or 133-136, wherein the guide region does not comprise a YA site at nucleotides 12-13 and nucleotide 12 is unmodified.

138. The gRNA of claim 124-132 or 134-137, wherein the guide region does not comprise a YA site at nucleotides 15-16 and nucleotide 15 is unmodified.

139. The gRNA of claim 124-133 or 135-138, wherein the guide region does not comprise a YA site at nucleotides 16-17 and nucleotide 16 is unmodified.

140. The gRNA of claim 124-134 or 136-139, wherein the guide region does not comprise a YA site at nucleotides 19-20 and nucleotide 19 is unmodified.

141. The gRNA of claims 124-140, wherein the short-sgRNA comprises a guide region that comprises one or more of the following:
i. 2′-OMe and phosphorothioate modifications at nucleotide 1;
ii. 2′-OMe and phosphorothioate modifications at nucleotide 2;
iii. 2′-OMe and phosphorothioate modifications at nucleotide 3;
iv. a 2′-OMe modification at nucleotide 4;
v. a phosphorothioate modification at nucleotide 6;
vi. a phosphorothioate modification at nucleotide 7;
vii. 2′-fluoro and phosphorothioate modifications at nucleotide 8;
viii. 2′-fluoro and phosphorothioate modifications at nucleotide 9;
ix. 2′-fluoro and phosphorothioate modifications at nucleotide 10;
x. a 2′-fluoro modification at nucleotide 11;
xi. a 2′-fluoro modifications at nucleotide 13;
xii. a 2′-fluoro modifications at nucleotide 14;
xiii. a 2′-fluoro modifications at nucleotide 17; and
xiv. a 2′-fluoro modifications at nucleotide 18.

142. The gRNA of claims 124-141, wherein the guide region comprises each of the modifications set forth in the preceding claim.

143. The gRNA of claims 124-142, wherein the guide region comprises at least 1, 2, 3, or 4 of the following:
i. a 2′-OMe modification at nucleotide 5 if nucleotides 5 and 6 form a YA site;
ii. a 2′-OMe modification at nucleotide 12 if nucleotides 12 and 13 form a YA site;
iii. a phosphorothioate modification at nucleotide 15 if nucleotides 15 and 16 form a YA site;
iv. a phosphorothioate modification at nucleotide 16 if nucleotides 16 and 17 form a YA site; and
v. a phosphorothioate or 2′-fluoro modification at nucleotide 19 if nucleotides 19 and 20 form a YA site.

144. The gRNA of claims 124-143, wherein the guide region comprises a YA site at nucleotides 5-6 and a 2′-OMe modification at nucleotide 5.

145. The gRNA of claims 124-144, wherein the guide region comprises a YA site at nucleotides 12-13 and a 2′-OMe modification at nucleotide 12.

146. The gRNA of claims 124-145, wherein the guide region comprises a YA site at nucleotides 15-16 and a phosphorothioate modification at nucleotide 15.

147. The gRNA of claims 124-146, wherein the guide region comprises a YA site at nucleotides 16-17 and a phosphorothioate modification at nucleotide 16.

148. The gRNA of claims 124-147, wherein the guide region comprises a YA site at nucleotides 19-20 and a phosphorothioate modification at nucleotide 19.

149. The gRNA of claims 124-148, wherein the guide region comprises a 2′-fluoro modification at nucleotide 19.

150. The gRNA of claims 124-149, wherein the guide region comprises an unmodified nucleotide 15 or only a phosphorothioate modification at nucleotide 15.

151. The gRNA of claims 124-150, wherein the guide region comprises an unmodified nucleotide 16 or only a phosphorothioate modification at nucleotide 16.

152. A guide RNA which is a single guide RNA (sgRNA) comprising:
i. a YA modification at two or more guide region YA sites;
ii. a YA modification at one or more of conserved region YA sites 2, 3, 4, and 10; and
iii. a YA modification at one or more of conserved region YA sites 1 and 8.

153. A guide RNA which is a single guide RNA (sgRNA) comprising:
i. a YA modification at one or more guide region YA sites that are at or after nucleotide 8 from the 5′ end of the 5′ terminus;
ii. a YA modification at one or more of conserved region YA sites 2, 3, 4, and 10; and optionally
iii. a YA modification at one or more of conserved region YA sites 1 and 8.

154. A guide RNA which is a single guide RNA (sgRNA) comprising:
i. a YA modification at one or more guide region YA sites that are within 13 nucleotides of the 3′ terminal nucleotide of the guide region;
ii. a YA modification at one or more of conserved region YA sites 2, 3, 4, and 10; and
iii. a YA modification at one or more of conserved region YA sites 1 and

155. A guide RNA which is a single guide RNA (sgRNA) comprising:
i. a 5′ end modification and a 3′ end modification;
ii. a YA modification at one or more of conserved region YA sites 2, 3, 4, and 10; and
iii. a YA modification at one or more of conserved region YA sites 1 and 8.

156. A guide RNA which is a single guide RNA (sgRNA) comprising:
i. a YA modification at at least one guide region YA site, wherein the modification of the guide region YA site comprises a modification at at least one nucleotide located 5′ of the guide region YA site does not comprise;
ii. a YA modification at one or more of conserved region YA sites 2, 3, 4, and 10; and
iii. a YA modification at one or more of conserved region YA sites 1 and 8.

157. A guide RNA which is a single guide RNA (sgRNA) comprising:
i. a YA modification at one or more of conserved region YA sites 2, 3, 4, and 10; and
ii. a YA modification at conserved region YA sites 1 and 8.

158. A guide RNA which is a single guide RNA (sgRNA) comprising:
i. a YA modification at one or more guide region YA sites that are at or after nucleotide 8 from the 5′ end of the 5′ terminus;
ii. a YA modification at one or more of conserved region YA sites 2, 3, 4, and 10; and
iii. a modification at one or more of H1-1 and H2-1.

159. A guide RNA which is a single guide RNA (sgRNA) comprising:
i. a YA modification at one or more of conserved region YA sites 2, 3, 4, and 10;
ii. a YA modification at one or more of conserved region YA sites 1, 5, 6, 7, 8, and 9; and
iii. a modification at one or more of H1-1 and H2-1.

160. A guide RNA which is an sgRNA comprising any one or more of the following:
i. a modification, such as a YA modification, at one or more nucleotides located at or after nucleotide 6 from the 5′ end of the 5′ terminus;
ii. a YA modification at one or more guide sequence YA sites;
iii. a modification at one or more of B3, B4, and B5, wherein B6 does not comprise a 2′-OMe modification or comprises a modification other than 2′-OMe;
iv. a modification at LS10, wherein LS10 comprises a modification other than 2′-fluoro; and/or
v. a modification at N2, N3, N4, N5, N6, N7, N10, or N11; and
wherein at least one of the following is true:
a. at least one of nucleotides 8-11, 13, 14, 17, or 18 from the 5′ end of the 5′ terminus does not comprise a 2′-fluoro modification;
b. at least one of nucleotides 6-10 from the 5′ end of the 5′ terminus does not comprise a phosphorothioate linkage;
c. at least one of B2, B3, B4, or B5 does not comprise a 2′-OMe modification;
d. at least one of LS1, LS8, or LS10 does not comprise a 2′-OMe modification;
e. at least one of N2, N3, N4, N5, N6, N7, N10, N11, N16, or N17 does not comprise a 2′-OMe modification;
f. H1-1 comprises a modification;
g. H2-1 comprises a modification; or
h. at least one of H1-2, H1-3, H1-4, H1-5, H1-6, H1-7, H1-8, H1-9, H1-10, H2-1, H2-2, H2-3, H2-4, H2-5, H2-6, H2-7, H2-8, H2-9, H2-10, H2-11, H2-12, H2-13, H2-14, or H2-15 does not comprise a phosphorothioate linkage.

161. A guide RNA comprising any one or more of the following:
i. a modification, such as a YA modification, at one or more nucleotides located at or after nucleotide 6 from the 5′ end of the 5′ terminus; or
ii. a YA modification at one or more guide sequence YA sites;
wherein at least one of the following is true:
a. at least one of nucleotides 8-11, 13, 14, 17, or 18 from the 5′ end of the 5′ terminus does not comprise a 2′-fluoro modification; or
b. at least one of nucleotides 6-10 from the 5′ end of the 5′ terminus does not comprise a phosphorothioate linkage;
and wherein at least one of the following is also true:
c. at least one of nucleotides 7-10 from the 5′ end of the 5′ terminus does not comprise a 2′-OMe modification;
d. nucleotide 20 from the 5′ end of the 5′ terminus does not comprise a 2′-OMe modification; or
e. the guide RNA comprises a 2′-fluoro modification at any one or more of nucleotides 1-20 from the 5′ end of the 5′ terminus and at least one of nucleotides 11, 12, 13, 14, 17, or 18 from the 5′ end of the 5′ terminus does not comprise a 2′-fluoro modification, optionally wherein nucleotide 12 from the 5′ end of the 5′ terminus does not comprise a 2′-fluoro modification.

162. A guide RNA which is an sgRNA comprising a guanosine at N14 and/or one or more of the following:
i. a YA modification at one or more guide region YA sites that are at or after nucleotide 8 from the 5′ end of the 5′ terminus;
ii. a YA modification at one or more of conserved region YA sites 1, 5, and 6, wherein if YA site 6 is modified at LS12 and LS9is not modified, then the modification of LS12 is other than 2′-OMe;
iii. a modification at LS9, wherein if LS9 is modified and LS5, LS7, and LS12 are not modified, then the modification of LS9 is other than 2′-fluoro,
iv. a modification at LS12, wherein if LS12 is modified and LS9 is not modified, then the modification of LS12 is other than 2′-OMe;
v. a modification at LS8 or LS11, wherein at least one of LS8 and LS11 comprises a modification other than 2′-OMe; and/or
vi. a modification at N6, N14, or N17, wherein if N17 is modified and N6 and N14 are not modified, then the modification of N17 is other than 2′-fluoro and other than 2′-OMe;
and wherein at least one of the following is true:
a. at least one of nucleotides 8-11, 13-14, 17, or 18 from the 5′ end of the 5′ terminus does not comprise a 2′-fluoro modification;
b. at least one of nucleotides 6-10 from the 5′ end of the 5′ terminus does not comprise a phosphorothioate linkage;
c. at least one of B2, B3, B4, or B5 does not comprise a 2′-OMe modification;
d. at least one of LS1, LS8, or LS10 does not comprise a 2′-OMe modification;
e. at least one of N2, N3, N4, N5, N6, N7, N10, N11, N16, or N17 does not comprise a 2′-OMe modification;
f. H1-1 comprises a modification;
g. H2-1 comprises a modification; or
h. at least one of H1-2, H1-3, H1-4, H1-5, H1-6, H1-7, H1-8, H1-9, H1-10, H2-1, H2-2, H2-3, H2-4, H2-5, H2-6, H2-7, H2-8, H2-9, H2-10, H2-11, H2-12, H2-13, H2-14, or H2-15 does not comprise a phosphorothioate linkage.

163. The gRNA of claim 161 or 162, comprising:
i. a YA modification at 1, 2, 3, 4, or 5 guide region YA sites;
ii. a YA modification at 1, 2, 3, 4, or 5 guide region YA sites, wherein the modification of at least one guide region YA site is different from any 5′ end modification of the sgRNA;
iii. a YA modification at one or more guide region YA sites that are at or after nucleotide 8 from the 5′ end of the 5′ terminus;
iv. a YA modification at one or more guide region YA sites that are is within nucleotides 5-end, 6-end, 7-end, 8-end, 9-end, or 10-end from the 5′ end of the 5′ terminus;
v. a YA modification at one or more guide region YA sites that are within 17, 16, 15, 14, 13, 12, 11, 10, or 9 nucleotides of the 3′ terminal nucleotide of the guide region;
vi. a YA modification at a guide region YA site other than a 5′ end modification; or
vii. a YA modification at a guide region YA site, wherein the modification of the guide region YA site comprises a modification at at least one nucleotide located 5′ of the guide region YA site does not comprise.

164. The gRNA of claim 163, comprising:
i. a YA modification at two or more guide region YA sites that are at or after nucleotide 8 from the 5′ end of the 5′ terminus;
ii. a YA modification at two or more guide region YA sites that are within nucleotides 5-end, 6-end, 7-end, 8-end, 9-end, or 10-end from the 5′ end of the 5′ terminus;
iii. a YA modification at two or more guide region YA sites that are within 17, 16, 15, 14, 13, 12, 11, 10, or 9 nucleotides of the 3′ terminal nucleotide of the guide region;
iv. a YA modification at two or more guide region YA sites other than a 5′ end modification; or
v. a YA modification at a two or more guide region YA sites, wherein the modifications of the guide region YA sites comprise a modification at at least one nucleotide located 5′ of the guide region YA site does not comprise.

165. The gRNA of claim 163, comprising:
i. a YA modification at three or more guide region YA sites that are at or after nucleotide 8 from the 5′ end of the 5′ terminus;
ii. a YA modification at three or more guide region YA sites that are within nucleotides 5-end, 6-end, 7-end, 8-end, 9-end, or 10-end from the 5′ end of the 5′ terminus;
iii. a YA modification at three or more guide region YA sites that are within 17, 16, 15, 14, 13, 12, 11, 10, or 9 nucleotides of the 3′ terminal nucleotide of the guide region;
iv. a YA modification at three or more guide region YA sites other than a 5′ end modification; or
v. a YA modification at a three or more guide region YA sites, wherein the modifications of the guide region YA sites comprise a modification at at least one nucleotide located 5′ of the guide region YA site does not comprise.

166. The gRNA of any one of claims 161-165, comprising at least one YA modification at nucleotide 6 from the 5′ end of the 5′ terminus.

167. The gRNA of any one of claims 161-166, comprising at least one YA modification at nucleotide 7 from the 5′ end of the 5′ terminus.

168. The gRNA of any one of claims 161-167, comprising at least one YA modification at nucleotide 8 from the 5′ end of the 5′ terminus.

169. The gRNA of any one of claims 161-168, comprising at least one YA modification at nucleotide 9 from the 5′ end of the 5′ terminus.

170. The gRNA of any one of claims 161-169, comprising at least one YA modification at nucleotide 10 from the 5′ end of the 5′ terminus.

171. The gRNA of any one of claims 161-170, comprising at least one YA modification at nucleotide 11 from the 5′ end of the 5′ terminus.

172. The gRNA of any one of claims 161-171, comprising at least one YA modification at nucleotide 12 from the 5′ end of the 5′ terminus.

173. The gRNA of any one of claims 161-172, comprising at least one YA modification at nucleotide 13 from the 5′ end of the 5′ terminus.

174. The gRNA of any one of claims 161-173, comprising at least one YA modification at nucleotide 14 from the 5′ end of the 5′ terminus.

175. The gRNA of any one of claims 161-174, comprising at least one YA modification at nucleotide 15 from the 5′ end of the 5′ terminus.

176. The gRNA of any one of claims 161-175, comprising at least one YA modification at nucleotide 16 from the 5′ end of the 5′ terminus.

177. The gRNA of any one of claims 161-176, comprising at least one YA modification at nucleotide 17 from the 5′ end of the 5′ terminus.

178. The gRNA of any one of claims 161-177, comprising at least one YA modification at nucleotide 18 from the 5′ end of the 5′ terminus.

179. The gRNA of any one of claims 161-178, comprising at least one YA modification at nucleotide 19 from the 5′ end of the 5′ terminus.

180. The gRNA of any one of claims 161-179, comprising at least one YA modification at nucleotide 20 from the 5′ end of the 5′ terminus.

181. The gRNA of any one of claims 161-180, wherein at least 1, 2, 3, 4, 5, 6, 7, or 8 of nucleotides 8-11, 13-14, and 17-18 from the 5′ end of the 5′ terminus comprise a YA modification, optionally wherein the modification comprises 2′-fluoro, 2′-H, 2′-OMe, or PS.

182. The gRNA of claim 181, wherein the modification is 2′-fluoro.

183. The gRNA of claim 181, wherein the modification is 2′-OMe or 2′-H.

184. The gRNA of claim 181, wherein the modification is PS.

185. The gRNA of any one of claims 161-184, wherein at least 1, 2, 3, 4, or 5 of nucleotides 6-10 from the 5′ end of the 5′ terminus comprise a YA modification, optionally wherein the modification comprises 2′-fluoro, 2′-H, 2′-OMe, inosine, or PS.

186. The gRNA of claim 185, wherein the modification is PS.

187. The gRNA of claim 185, wherein the modification is 2′-fluoro or 2′-H.

188. The gRNA of claim 185, wherein the modification is 2′-OMe.

189. The gRNA of any one of claims 161-188, comprising any one or more of the following:
i. 1, 2, 3, 4, 5, 6, 7, or 8 YA modifications of nucleotides 8-11, 13-14, and 17-18 from the 5′ end of the 5′ terminus, wherein the YA modifications are optionally 2′-fluoro modifications, and a modification other than 2′-fluoro at one or more of nucleotides 6-10 from the 5′ terminus;
ii. a YA modification other than PS at one or more of nucleotides 8-11, 13-14, and 17-18 from the 5′ end of the 5′ terminus, and 1, 2, 3, 4, or 5 YA modifications at nucleotides 6-10 from the 5′ end of the 5′ terminus, optionally wherein the modifications are PS modifications;
iii. 1, 2, 3, 4, 5, 6, 7, or 8 YA modifications at nucleotides 8-11, 13-14, and 17-18 from the 5′ end of the 5′ terminus, wherein the YA modifications are optionally 2′-fluoro modifications, and modifications other than 2′-fluoro at nucleotides 6-10 from the 5′ end of the 5′ terminus;
iv. YA modifications other than PS at each of nucleotides 8-11, 13-14, and 17-18 from the 5′ end of the 5′ terminus, and 1, 2, 3, 4, or 5 YA modifications at nucleotides 6-10 from the 5′ end of the 5′ terminus, wherein the modifications are optionally PS modifications;
v. 1, 2, 3, 4, 5, 6, 7, or 8 YA modifications at nucleotides 8-11, 13-14, and 17-18 from the 5′ end of the 5′ terminus, wherein the YA modifications are optionally 2′-fluoro modifications, and one or more PS modification at any one of nucleotides 6-10 from the 5′ end of the 5′ terminus;
vi. at least one 2′-fluoro modification at any one of nucleotides 8-11, 13-14, and 17-18 from the 5′ end of the 5′ terminus, and 1, 2, 3, 4, or 5 YA modifications of nucleotides 6-10 from the 5′ end of the 5′ terminus, wherein the modifications are optionally PS modifications;
vii. 1, 2, 3, 4, 5, 6, 7, or 8 YA modifications of nucleotides 8-11, 13-14, and 17-18 from the 5′ end of the 5′ terminus, wherein the YA modifications are optionally 2′-fluoro modifications, and a PS modification at each of nucleotides 6-10 from the 5′ end of the 5′ terminus; or
viii. a 2′-fluoro modification at each of nucleotides 8-11, 13-14, and 17-18 from the 5′ end of the 5′ terminus, and 1, 2, 3, 4, or 5 YA modifications of nucleotides 6-10 from the 5′ end of the 5′ terminus, wherein the modifications are optionally PS modifications.

190. The gRNA of any one of claims 161-189, wherein:
i. nucleotides 4-20 from the 5′ end of the 5′ terminus comprise at least 2, 3, or 4 modified YA sites including a first modified YA site comprising a 2′-OMe modification and a second modified YA site comprising a 2′-fluoro modification or a PS modification;
ii. nucleotides 4-20 from the 5′ end of the 5′ terminus comprise at least 2, 3, or 4 modified YA sites including a first modified YA site comprising a 2′-fluoro modification and a second modified YA site comprising a 2′-OMe modification or a PS modification;
iii. nucleotides 4-20 from the 5′ end of the 5′ terminus comprise at least 2, 3, or 4 modified YA sites including a first modified YA site comprising a PS modification and a second modified YA site comprising a 2′-OMe modification or a 2′-fluoro modification;
iv. nucleotides 4-20 from the 5′ end of the 5′ terminus comprise at least 2, 3, or 4 modified YA sites including a YA modification;
v. nucleotides 4-20 from the 5′ end of the 5′ terminus comprise at least 3 or 4 modified YA sites including a first modified YA site comprising a 2′-OMe modification, a second modified YA site comprising a 2′-fluoro modification, and a third modified YA site comprising a PS modification;
vi. nucleotides 4-20 from the 5′ end of the 5′ terminus comprise at least 3 or 4 modified YA sites including a first modified YA site comprising a 2′-OMe modification, a second modified YA site comprising a 2′-fluoro modification, a third modified YA site comprising a 2′-fluoro modification, and a fourth modified YA site comprising a PS modification;
vii. nucleotides 4-20 from the 5′ end of the 5′ terminus comprise at least 3 or 4 modified YA sites including a YA modification;
viii. nucleotides 4-20 from the 5′ end of the 5′ terminus comprise at least 4 modified YA sites including a first modified YA site comprising a 2′-OMe modification, a second modified YA site comprising a 2′-fluoro modification, a third modified YA site comprising a PS modification, and a fourth modified YA site comprising a PS modification; or
ix. nucleotides 4-40 from the 5′ end of the 5′ terminus comprise at least 4 modified YA sites including a YA modification.

191. The gRNA of any one of claims 161-190, wherein nucleotides 4-20 from the 5′ end of the 5′ terminus comprise at least 5 modified YA sites.

192. The gRNA of any one of claims 161-191, wherein the at least 5 modified YA sites include a fifth modified YA site comprising a PS modification, optionally wherein the third modified YA site comprises a 2′-fluoro modification.

193. The gRNA of any one of claims 161-192, wherein the first, second, and (if applicable) third, fourth, and fifth of the at least 5 modified YA sites are arranged in the 5′ to 3′ direction.

194. The gRNA of any one of claims 161-193, wherein the first, second, and (if applicable) third, fourth, and fifth of the at least 5 modified YA sites are not arranged in the 5′ to 3′ direction.

195. The gRNA of any one of claims 161-194, wherein nucleotides 4-20 from the 5′ end of the 5′ terminus comprise at least 2, 3, 4, or 5 modified YA sites comprising a deoxyribonucleotide, optionally wherein the deoxyribonucleotide is the pyrimidine of the YA sites.

196. The gRNA of any one of claims 161-195, wherein:
i. at least 1, 2, 3, or 4 of nucleotides 8-11 from the 5′ end of the 5′ terminus comprise a YA modification, which is optionally a 2′-fluoro modification;
ii. at least 1, 2, 3, 4, 5, 6, 7, or 8 of nucleotides 8-11, 13, 14, 17, and 18 from the 5′ end of the 5′ terminus comprise a YA modification, optionally wherein the YA modifications are 2′-OMe if present at nucleotides 8-11 and 2′-fluoro if present at nucleotides 13, 14, 17, or 18;
iii. at least one or both of nucleotides 17 and 18 from the 5′ end of the 5′ terminus comprise a YA modification, which is optionally a 2′-fluoro modification;
iv. at least one or both of nucleotides 17 and 18 from the 5′ end of the 5′ terminus comprise a YA modification, which is optionally a 2′-fluoro modification; or
v. at least 1, 2, 3, 4, 5, 6, 7, 8, 9, 10, 11, 12, or 13 of nucleotides 4-14, 17, and 18 from the 5′ end of the 5′ terminus comprise a YA modification, which is optionally a 2′-fluoro modification.

197. The gRNA of any one of claims 161-196, wherein at least 1, 2, 3, 4, 5, or 6 of nucleotides 4-10 from the 5′ end of the 5′ terminus comprise a YA modification, which is optionally a 2′-OMe modification.

198. The gRNA of any one of claims 161-197, wherein nucleotides 4-10 from the 5′ end of the 5′ terminus comprise a YA modification, which is optionally a 2′-OMe modification.

199. The gRNA of any one of claims 161-198, wherein:
i. at least one of nucleotides 1-3 from the 5′ end of the 5′ terminus comprise a 5′ protective end modification, which is optionally a 2′-OMe modification;
ii. at least two of nucleotides 1-3 from the 5′ end of the 5′ terminus comprise a 5′ protective end modification, which is optionally a 2′-OMe modification; or
iii. each of nucleotides 1-3 from the 5′ end of the 5′ terminus comprise a 5′ protective end modification, which is optionally a 2′-OMe modification.

200. The gRNA of any one of claims 161-199, wherein at least 1, 2, 3, 4, or 5 of nucleotides 11, 13, 14, 17, and 18 from the 5′ end of the 5′ terminus comprise a 5′ end modification, which is optionally a 2′-fluoro modification.

201. The gRNA of any one of claims 161-200, wherein nucleotide 15 from the 5′ end of the 5′ terminus is unmodified or modified only with phosphorothioate.

202. The gRNA of any one of claims 161-200, wherein nucleotide 16 from the 5′ terminus is unmodified or modified only with phosphorothioate.

203. The gRNA of any one of the preceding claims, wherein nucleotide 3 from the 5′ end of the 5′ terminus is unmodified or modified only with phosphorothioate.

204. The gRNA of any one of claims 161-203, which is a crRNA or dgRNA.

205. The gRNA of any one of claims 161-203, which is an sgRNA.

206. The gRNA of any one of claims 161-203, which is a short-sgRNA.

207. The gRNA of any one of claim 205 or 206, comprising a YA modification of conserved region YA site 1.

208. The gRNA of any one of claims 205-207, comprising a YA modification of conserved region YA site 2.

209. The gRNA of any one of claims 205-208, comprising a YA modification of conserved region YA site 3.

210. The gRNA of any one of claims 205-209, comprising a YA modification of conserved region YA site 4.

211. The gRNA of any one of claims 205-210, comprising a YA modification of conserved region YA site 5.

212. The gRNA of any one of claims 205-211, comprising a YA modification of conserved region YA site 6.

213. The gRNA of any one of claims 205-212, comprising a YA modification of conserved region YA site 7.

214. The gRNA of any one of claims 205-213, comprising a YA modification of conserved region YA site 8.

215. The gRNA of any one of claims 205-214, comprising a YA modification of conserved region YA site 9.

216. The gRNA of any one of claims 205-215, comprising a YA modification of conserved region YA site 10.

217. The gRNA of any one of claims 205-216, comprising:
i. YA modifications of conserved region YA sites 2, 3, 4, and 10;
ii. YA modifications of conserved region YA sites 2, 3, and 4;
iii. YA modifications of conserved region YA sites 2, 3, and 10;
iv. YA modifications of conserved region YA sites 2, 4, and 10;
v. YA modifications of conserved region YA sites 3, 4, and 10;
vi. YA modifications of conserved region YA sites 2 and 10;
vii. YA modifications of conserved region YA sites 2 and 4;
viii. YA modifications of conserved region YA sites 2 and 3;
ix. YA modifications of conserved region YA sites 3 and 4;
x. YA modifications of conserved region YA sites 3 and 10; or
xi. YA modifications of conserved region YA sites 4 and 10.

218. The gRNA of any one of claims 205-217, comprising:
i. YA modifications of conserved region YA sites 1 and 5;
ii. YA modifications of conserved region YA sites 1 and 6;
iii. YA modifications of conserved region YA sites 1 and 7;
iv. YA modifications of conserved region YA sites 1 and 8;
v. YA modifications of conserved region YA sites 1 and 9;
vi. YA modifications of conserved region YA sites 8 and 5;
vii. YA modifications of conserved region YA sites 8 and 6;
viii. YA modifications of conserved region YA sites 8 and 7; or
ix. YA modifications of conserved region YA sites 8 and 9;
optionally wherein the sgRNA further comprises YA modifications of conserved region YA sites 2, 3, 4, and 10.

219. The gRNA of any one of claims 205-218, wherein at least one modified YA site comprises a 2′-OMe modification, optionally at the pyrimidine of the YA site.

220. The gRNA of any one of claims 205-219, wherein at least one modified YA site comprises a 2′-fluoro modification, optionally at the pyrimidine of the YA site.

221. The gRNA of any one of claims 205-220, wherein at least one modified YA site comprises a PS modification, optionally at the pyrimidine of the YA site.

222. The gRNA of any one of claims 205-221, wherein at least 2, 3, 4, 5, 6, 7, 8, 9, or 10 modified YA sites comprise a 2′-OMe modification, optionally at the pyrimidines of the YA sites.

223. The gRNA of any one of claims205-222, wherein at least 2, 3, 4, 5, 6, 7, 8, 9, or 10 modified YA sites comprise a 2′-fluoro modification, optionally at the pyrimidines of the YA sites.

224. The gRNA of any one of claims205-223, wherein at least 2, 3, 4, 5, 6, 7, 8, 9, or 10 modified YA sites comprise a PS modification, optionally at the pyrimidines of the YA sites.

225. The gRNA of any one of claims205-224, wherein at least 2, 3, 4, 5, 6, 7, 8, 9, or 10 modified YA sites comprise a ribose modification at the 2′ position, optionally at the pyrimidines of the YA sites, and optionally chosen from a 2′-O-alkyl, 2′-H, and 2′-fluoro modification.

226. The gRNA of any one of claims 205-225, wherein:
i. conserved region YA sites 1 and 8 comprise 2′-fluoro modifications, optionally at the pyrimidines of the YA sites;
ii. conserved region YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites;
iii. conserved region YA site 1 comprises a 2′-fluoro modification and conserved region YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites;
iv. conserved region YA site 8 comprises a 2′-fluoro modification and conserved region YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites;
v. conserved region YA site 1 comprises a 2′-fluoro modification at the pyrimidine of the YA sites and YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites;
vi. conserved region YA site 8 comprises a 2′-fluoro modification at the pyrimidine of the YA site and YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites;
vii. conserved region YA sites 1 and 8 comprise 2′-fluoro modifications and conserved region YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites; or
viii. conserved region YA sites 1 and 8 comprise 2′-fluoro modifications at the pyrimidines of the YA sites and conserved region YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites.

227. The gRNA of any one of claims 205-226, wherein conserved region YA sites 7 and 9 comprise YA modifications, which are optionally 2′-OMe modifications.

228. The gRNA of any one of claims 205-227, wherein conserved region YA sites 5, 6, 7, and 9 comprise YA modifications, which are optionally 2′-OMe modifications.

229. The gRNA of any one of claims 205-228, wherein conserved region YA site 8 comprises a 2′-fluoro modification.

230. The gRNA of any one of claims 205-229, wherein conserved region YA site 8 comprises a deoxyribonucleotide modification.

231. The gRNA of any one of claims 205-230, wherein conserved region YA site 8 is abolished by a base substitution, optionally wherein the base substitution eliminates the uracil of YA site 8, further optionally wherein the base substitution is a uracil to guanine substitution.

232. The gRNA of any one of claims 205-231, wherein conserved region YA site 1 comprises a 2′-fluoro modification.

233. The gRNA of any one of claims 205-232, wherein conserved region YA site 1 comprises a PS modification.

234. The gRNA of any one of claims 205-233, wherein 1, 2, 3, 4, 5, 6, or 7 of LS5, LS7, LS8, LS9, LS10, LS11, and LS12 comprise modifications, optionally wherein the modifications are 2′-fluoro and/or 2′-OMe modifications.

235. The gRNA of any one of claims 205-234, wherein modifications at LS5, LS7, LS9, and LS11, if present, comprise 2′-fluoro modifications, optionally wherein each of LS5, LS7, LS9, and LS11 comprise 2′-fluoro modifications.

236. The gRNA of any one of claims 205-235, wherein modifications at LS8, LS10, and LS12, if present, comprise 2′-OMe modifications, optionally wherein each of LS8, LS10, and LS12 comprise 2′-OMe modifications.

237. The gRNA of any one of claims 205-236, wherein 1, 2, 3, 4, 5, 6, 7, 8, 9, or 10 of N2, N3, N4, N5, N6, N7, N10, N11, N16, and N17 comprise modifications, which are optionally 2′-OMe modifications.

238. The gRNA of any one of claims 205-237, wherein H2-2 comprises a modification, optionally wherein H2 is otherwise unmodified.

239. The gRNA of any one of claims 205-238, wherein H2-2 comprises a 2′-OMe modification.

240. The gRNA of any one of claims 205-239, wherein US3, US9, and US12 comprise modifications, optionally wherein the US is otherwise unmodified.

241. The gRNA of any one of claims 205-240, wherein US3, US9, and US12 comprise 2′-OMe modifications.

242. The gRNA of any one of claims 205-241, wherein nucleotides 6-10 from the 5′ end of the 5′ terminus comprise a PS modification and nucleotides 8-11, 13, 14, 17, and 18 from the 5′ end of the 5′ terminus comprise a 2′-fluoro modification.

243. The gRNA of any one of claims 205-242, wherein each guide region YA site comprises a 2′-fluoro modification, optionally excepting nucleotides 15 and/or 16 from the 5′ end of the 5′ terminus.

244. The gRNA of any one of claims 205-243, wherein nucleotides 4, 8, and 11 from the 5′ end of the 5′ terminus comprise YA modifications, optionally wherein nucleotide 4 comprises a 2′-OMe modification and nucleotides 8 and 11 comprise a 2′-fluoro modification.

245. The gRNA of any one of claims 205-244, wherein 1, 2, 3, 4, 5, 6, 7, 8, 9, 10, 11, 12, 13, 14, 15, or more modified YA sites comprise a YA modification at the pyrimidine position of the YA site.

246. The gRNA of claim 245, wherein 1, 2, 3, 4, 5, 6, 7, 8, 9, or 10 modified conserved region YA sites comprise a YA modification at the pyrimidine position of the YA site.

247. The gRNA of any one of claims 205-246, wherein 1, 2, 3, 4, 5, 6, 7, 8, 9, 10, 11, 12, 13, 14, 15, or more modified YA sites comprise a YA modification at the adenine position of the YA site.

248. The gRNA of claim 247, wherein 1, 2, 3, 4, 5, 6, 7, 8, 9, or 10 modified conserved region YA sites comprise a YA site modification at the adenine position of the YA site.

249. The gRNA of any one of claims 205-248, comprising:
i. a modification of H1-1;
ii. a modification of H2-1; or
iii. modifications of H1-1 and H2-1.

250. The gRNA of claim 249, wherein H1-1 and/or H2-1 comprises a 2′-OMe modification.

251. The gRNA of claim 250, wherein H1-1 and/or H2-1 comprises a 2′-fluoro modification.

252. The gRNA of claim 251, wherein H1-1 and/or H2-1 comprises a PS modification.

253. The gRNA of any one of claims 205-252, comprising a modification at B3, optionally wherein B6 does not comprise a 2′-OMe modification or comprises a modification other than 2′-OMe.

254. The gRNA of any one of claims 205-253, comprising a modification at B4, optionally wherein B6 does not comprise a 2′-OMe modification or comprises a modification other than 2′-OMe.

255. The gRNA of any one of claims 205-254, comprising a modification at B5, optionally wherein B6 does not comprise a 2′-OMe modification or comprises a modification other than 2′-OMe.

256. The gRNA of any one of claims 205-255, comprising a modification at LS10, optionally wherein LS10 comprises a modification other than 2′-fluoro.

257. The gRNA of any one of claims 205-256, comprising a modification at N2.

258. The gRNA of any one of claims 205-257, comprising a modification at N3.

259. The gRNA of any one of claims 205-258, comprising a modification at N4.

260. The gRNA of any one of claims 205-259, comprising a modification at N5.

261. The gRNA of any one of claims 205-260, comprising a modification at N6.

262. The gRNA of any one of claims 205-261, comprising a modification at N7.

263. The gRNA of any one of claims 205-262, comprising a modification at N10.

264. The gRNA of any one of claims 205-263, comprising a modification at N11.

265. The gRNA of any one of claims 205-264, wherein:
i. nucleotide 8 from the 5′ end of the 5′ terminus does not comprise a 2′-fluoro modification;
ii. nucleotide 9 from the 5′ end of the 5′ terminus does not comprise a 2′-fluoro modification;
iii. nucleotide 10 from the 5′ end of the 5′ terminus does not comprise a 2′-fluoro modification;
iv. nucleotide 11 from the 5′ end of the 5′ terminus does not comprise a 2′-fluoro modification;
v. nucleotide 13 from the 5′ end of the 5′ terminus does not comprise a 2′-fluoro modification;
vi. nucleotide 14 from the 5′ end of the 5′ terminus does not comprise a 2′-fluoro modification;
vii. nucleotide 17 from the 5′ end of the 5′ terminus does not comprise a 2′-fluoro modification; and/or
viii. nucleotide 18 from the 5′ end of the 5′ terminus does not comprise a 2′-fluoro modification.

266. The gRNA of any one of claims 205-265, wherein:
i. nucleotide 6 from the 5′ end of the 5′ terminus does not comprise a 2′-fluoro modification;
ii. nucleotide 7 from the 5′ end of the 5′ terminus does not comprise a 2′-fluoro modification;
iii. nucleotide 8 from the 5′ end of the 5′ terminus does not comprise a 2′-fluoro modification;
iv. nucleotide 9 from the 5′ end of the 5′ terminus does not comprise a 2′-fluoro modification; and/or
v. nucleotide 10 from the 5′ end of the 5′ terminus does not comprise a 2′-fluoro modification.

267. The gRNA of any one of claims 205-266, wherein:
i. nucleotide 6 from the 5′ end of the 5′ terminus does not comprise a phosphorothioate linkage;
ii. nucleotide 7 from the 5′ end of the 5′ terminus does not comprise a phosphorothioate linkage;
iii. nucleotide 8 from the 5′ end of the 5′ terminus does not comprise a phosphorothioate linkage;
iv. nucleotide 9 from the 5′ end of the 5′ terminus does not comprise a phosphorothioate linkage; and/or
v. nucleotide 10 from the 5′ end of the 5′ terminus does not comprise a phosphorothioate linkage.

268. The gRNA of any one of claims 205-267, wherein:
i. nucleotide 7 from the 5′ end of the 5′ terminus does not comprise a 2′-OMe modification;
ii. nucleotide 8 from the 5′ end of the 5′ terminus does not comprise a 2′-OMe modification;
iii. nucleotide 9 from the 5′ end of the 5′ terminus does not comprise a 2′-OMe modification; and/or
iv. nucleotide 10 from the 5′ end of the 5′ terminus does not comprise a 2′-OMe modification.

269. The gRNA of any one of claims 205-268, wherein nucleotide 20 from the 5′ end of the 5′ terminus does not comprise a 2′-OMe modification.

270. The gRNA of any one of claims 205-269, wherein the guide RNA comprises a 2′-fluoro modification at any one or more of nucleotides 1-11 and 13-20 from the 5′ end of the 5′ terminus and nucleotide 12 from the 5′ end of the 5′ terminus does not comprise a 2′-fluoro modification.

271. The gRNA of any one of claims 205-270, wherein the guide RNA comprises a 2′-fluoro modification at any one or more of nucleotides 1-20 from the 5′ end of the 5′ terminus and:
i. nucleotide 11 from the 5′ end of the 5′ terminus does not comprise a 2′-fluoro modification;
ii. nucleotide 12 from the 5′ end of the 5′ terminus does not comprise a 2′-fluoro modification;
iii. nucleotide 13 from the 5′ end of the 5′ terminus does not comprise a 2′-fluoro modification;
iv. nucleotide 14 from the 5′ end of the 5′ terminus does not comprise a 2′-fluoro modification;
v. nucleotide 17 from the 5′ end of the 5′ terminus does not comprise a 2′-fluoro modification; and/or
vi. nucleotide 18 from the 5′ end of the 5′ terminus does not comprise a 2′-fluoro modification.

272. The gRNA of any one of claims 205-271, wherein:
i. B2 does not comprise a 2′-OMe modification;
ii. B3 does not comprise a 2′-OMe modification;
iii. B4 does not comprise a 2′-OMe modification; and/or
iv. B5 does not comprise a 2′-OMe modification.

273. The gRNA of any one of claims 205-272, wherein:
i. LS1 does not comprise a 2′-OMe modification;
ii. LS8 does not comprise a 2′-OMe modification; and/or
iii. LS10 does not comprise a 2′-OMe modification.

274. The gRNA of any one of claims 205-273, wherein:
i. N2 does not comprise a 2′-OMe modification;
ii. N3 does not comprise a 2′-OMe modification;
iii. N4 does not comprise a 2′-OMe modification;
iv. N5 does not comprise a 2′-OMe modification;
v. N6 does not comprise a 2′-OMe modification;
vi. N7 does not comprise a 2′-OMe modification;
vii. N10 does not comprise a 2′-OMe modification;
viii. N11 does not comprise a 2′-OMe modification;
ix. N16 does not comprise a 2′-OMe modification; and/or
x. N17 does not comprise a 2′-OMe modification.

275. The gRNA of any one of claims 205-274, wherein:
i. H1-2 does not comprise a phosphorothioate linkage;
ii. H1-3 does not comprise a phosphorothioate linkage;
iii. H1-4 does not comprise a phosphorothioate linkage;
iv. H1-5 does not comprise a phosphorothioate linkage;
v. H1-6 does not comprise a phosphorothioate linkage;
vi. H1-7 does not comprise a phosphorothioate linkage;
vii. H1-8 does not comprise a phosphorothioate linkage;
viii. H1-9 does not comprise a phosphorothioate linkage;
ix. H1-10 does not comprise a phosphorothioate linkage;
x. H2-1 does not comprise a phosphorothioate linkage;
xi. H2-2 does not comprise a phosphorothioate linkage;
xii. H2-3 does not comprise a phosphorothioate linkage;
xiii. H2-4 does not comprise a phosphorothioate linkage;
xiv. H2-5 does not comprise a phosphorothioate linkage;
xv. H2-6 does not comprise a phosphorothioate linkage;
xvi. H2-7 does not comprise a phosphorothioate linkage;
xvii. H2-8 does not comprise a phosphorothioate linkage;
xviii. H2-9 does not comprise a phosphorothioate linkage;
xix. H2-10 does not comprise a phosphorothioate linkage;
xx. H2-11 does not comprise a phosphorothioate linkage;
xxi. H2-12 does not comprise a phosphorothioate linkage;
xxii. H2-13 does not comprise a phosphorothioate linkage;
xxiii. H2-14 does not comprise a phosphorothioate linkage; and/or
xxiv. H2-15 does not comprise a phosphorothioate linkage.

276. A gRNA which is an sgRNA comprising modifications at:
i. nucleotides 6-10 from the 5′ end of the 5′ terminus, which are optionally PS modifications;
ii. nucleotides 8-11, 13, 14, 17, and 18 from the 5′ end of the 5′ terminus, which are optionally 2′-fluoro modifications; and
iii. H1-1 and H2-1, which are optionally 2′-OMe modifications, or conserved region YA site 1 or 8.

277. A gRNA which is an sgRNA comprising YA modifications at:
i. conserved region YA sites 1, 5, 6, 7, and 9, which are optionally 2′-OMe modifications; and
ii. conserved region YA site 8, which is optionally a 2′-fluoro modification.

278. A gRNA comprising YA modifications at four guide region YA sites, wherein at least one of the YA sites is at or after nucleotide 8 from the 5′ end of the 5′ terminus, and wherein:
i. the first YA site comprises a 2′-OMe modification;
ii. the second YA site comprises a 2′-fluoro modification;
iii. the third YA site comprises a 2′-fluoro or PS modification; and
iv. the fourth YA site comprises a PS modification,
optionally wherein the first, second, third, and fourth YA sites are arranged in the 5′ to 3′ direction.

279. The gRNA of claim 278, wherein the third YA site comprises a PS modification.

280. The gRNA of any one of claims 278-279, wherein the third YA site comprises a 2′-fluoro modification.

281. The gRNA of any one of claims 278-280, further comprising a fifth YA site comprising a PS modification, which is optionally 3′ of the fourth YA site.

282. The gRNA of any one of the claims 205-281, wherein conserved region YA sites 1, 5, 6, 7, and 9 comprise YA modifications, which are optionally 2′-OMe modifications; and conserved region YA site 8 comprises a modification, which is optionally a 2′-fluoro modification.

283. A gRNA which is an sgRNA comprising YA modifications at:
i. nucleotide 4 from the 5′ end of the 5′ terminus, wherein the YA modification is optionally a 2′-OMe modification;
ii. nucleotides 6-10 from the 5′ end of the 5′ terminus, which are optionally PS modifications;
iii. nucleotides 8-11, 13, 14, 17, and 18 from the 5′ end of the 5′ terminus, which are optionally 2′-fluoro modifications;
iv. LS5, LS7, LS9, and LS11, which are optionally 2′-fluoro modifications;
v. LS8, LS10, and LS12, which are optionally 2′-OMe modifications;
vi. N2, N3, N4, N5, N6, N7, N10, N11, N16, and N17, which are optionally 2′-OMe modifications; and
vii. N14, which is optionally a 2′-fluoro modification.

284. The gRNA of any one of claim 161-, wherein one or more of the following are true:
i. nucleotide 4 from the 5′ end of the 5′ terminus comprises a 2′-OMe modification;
ii. nucleotides 6-10 from the 5′ end of the 5′ terminus comprise PS modifications;
iii. nucleotides 8-11, 13, 14, 17, and 18 from the 5′ end of the 5′ terminus comprise 2′-fluoro modifications;
iv. LS5, LS7, LS9, and LS11 comprise 2′-fluoro modifications;
v. LS8, LS10, and LS12 comprise 2′-OMe modifications;
vi. N2, N3, N4, N5, N6, N7, N10, N11, N16, and N17 comprise 2′-OMe modifications; and
vii. N14 comprises a 2′-fluoro modification.

285. The gRNA of any one of claims 161-284, wherein at least one YA modification comprises a modification of the pyrimidine position of the YA site.

286. The gRNA of any one of claims 161-285, wherein at least one YA modification comprises a modification of the adenine position of the YA site.

287. The gRNA of any one of claims 161-286, wherein at least 2, 3, 4, 5, 6, 7, 8, 9, or 10 YA sites comprise YA modifications at the pyrimidines positions of the YA sites.

288. The gRNA of any one of claims 161-287, wherein at least 2, 3, 4, 5, 6, 7, 8, 9, or 10 YA sites comprise YA modifications at the adenine positions of the YA sites.

289. The gRNA of any one of claims 161-288, wherein at least one YA modification comprises a 2′-OMe modification.

290. The gRNA of any one of claims 161-289, wherein at least 2, 3, 4, 5, 6, 7, 8, 9, or 10 YA sites comprise a 2′-OMe modification.

291. The gRNA of any one of claims 161-290, wherein each modified conserved region YA site comprises a modification at the pyrimidine position of the YA site.

292. The gRNA of any one of claims 161-291, wherein each modified guide region YA site, or each modified conserved region and guide region YA site, comprises a modification at the pyrimidine position of the YA site.

293. The gRNA of any one of claims 161-292, wherein each modified conserved region YA site comprises a modification at the adenine position of the YA site.

294. The gRNA of any one of claims 161-293, wherein each modified guide region YA site, or each modified conserved region and guide region YA site, comprises a modification at the adenine position of the YA site.

295. The gRNA of any one of claims 161-294, which is an sgRNA comprising a modification at LS5.

296. The gRNA of any one of claims 161-295, which is an sgRNA comprising a modification at LS7.

297. The gRNA of any one of claims 161-296, which is an sgRNA comprising a modification at LS9, optionally wherein if LS9 is modified and LS5, LS7, and LS12 are not, then the modification of LS9 is other than 2′-fluoro.

298. The gRNA of any one of claims 161-297, which is an sgRNA comprising a modification at LS12, optionally wherein if LS12 is modified and LS9 is not, then the modification of LS12 is other than 2′-OMe.

299. The gRNA of any one of claims 161-298, which is an sgRNA comprising at least one YA modification that stabilizes a secondary structure, optionally wherein the secondary structure is the lower stem.

300. The gRNA of any one of claims 161-299, which is an sgRNA comprising at least one modification of LS8 and/or LS11, optionally wherein the modification of LS8 and/or LS11 stabilizes a secondary structure.

301. The gRNA of any one of claims 161-300, comprising a YA modification that stabilizes a secondary structure chosen from:
i. ENA;
ii. LNA; or
iii. a bicyclic ribose modification.

302. The gRNA of any one of claims 161-301, which is an sgRNA comprising a modification at N6.

303. The gRNA of any one of claims 161-302, which is an sgRNA comprising a modification at N14.

304. The gRNA of any one of claims 161-303, which is an sgRNA comprising a modification at N17, optionally wherein if N17 is modified and N6 and N14 are not, then the modification of N17 is other than 2′-fluoro and other than 2′-OMe.

305. The gRNA of any one of claims 161-304, wherein at least 1, 2, or 3 of nucleotides 1-3 from the 5′ end of the 5′ terminus comprise deoxyribonucleotides, optionally wherein nucleotides 1-3 from the 5′ end of the 5′ terminus comprise PS modifications.

306. The gRNA of any one of claims 161-305, wherein the gRNA is an sgRNA and at least 1, 2, or 3 of nucleotides 1-3 from the 3′ end of the 3′ terminus comprise deoxyribonucleotides, optionally wherein nucleotides 2-3 from the 3′ end of the 3′ terminus comprise PS modifications.

307. The gRNA of any one of claims 161-306, wherein the gRNA is an sgRNA and nucleotide 4 from the 3′ end of the 3′ terminus comprises a PS modification, optionally wherein nucleotide 4 from the 3′ end of the 3′ terminus comprises a 2′-OMe modification.

308. The gRNA of any one of claims 161-307, wherein the gRNA is an sgRNA and hairpin 2 comprises deoxyribonucleotides, optionally wherein all or all but 1, 2, 3, 4, 5, 6, 7, 8, 9, or 10 nucleotides of hairpin 1 and hairpin 2 are deoxyribonucleotides.

309. The gRNA of any one of claims 161-308, wherein the gRNA is an sgRNA and hairpin 1 and hairpin 2 comprise deoxyribonucleotides, optionally wherein all or all but 1, 2, 3, 4, 5, 6, 7, 8, 9, 10, 11, 12, or 13 nucleotides of hairpin 1 and hairpin 2 are deoxyribonucleotides.

310. The gRNA of any one of claims 161-309, wherein the gRNA is an sgRNA and all or all but 1, 2, 3, 4, 5, 6, 7, 8, 9, 10, 11, 12, or 13 nucleotides from the beginning of hairpin 1 to the 3′ end of the sgRNA are deoxyribonucleotides, optionally wherein nucleotides 1-3 from the 3′ end of the 3′ terminus are deoxyribonucleotides.

311. The gRNA of any one of claims 161-310, wherein the gRNA is an sgRNA and the upper stem comprises deoxyribonucleotides.

312. The gRNA of any one of claims 161-311, wherein the gRNA is an sgRNA and all or all but 1, 2, 3, 4, 5, 6, 7, 8, 9, or 10 nucleotides of the upper stem are deoxyribonucleotides.

313. The gRNA of any one of claims 161-312, wherein at least 1, 2, or 3 of nucleotides 1-3 from the 5′ end of the 5′ terminus comprise ENA, optionally wherein nucleotides 1-3 from the 5′ end of the 5′ terminus comprise PS modifications.

314. The gRNA of any one of claims 161-313, wherein the gRNA is an sgRNA and at least 1, 2, or 3 of nucleotides 2-4 from the 3′ end of the 3′ terminus comprise ENA, optionally wherein nucleotides 2-3 from the 3′ end of the 3′ terminus comprise PS modifications.

315. The gRNA of any one of claims 161-314, wherein at least 1, 2, or 3 of nucleotides 1-3 from the 5′ end of the 5′ terminus comprise UNA, optionally wherein nucleotides 1-3 from the 5′ end of the 5′ terminus comprise PS modifications.

316. The gRNA of any one of claims 161-315, wherein the gRNA is an sgRNA and at least 1, 2, or 3 of nucleotides 2-4 from the 3′ end of the 3′ terminus comprise UNA, optionally wherein nucleotides 2-3 from the 3′ end of the 3′ terminus comprise PS modifications.

317. The gRNA of any one of claims 161-316, wherein the gRNA is an sgRNA and nucleotide 4 from the 3′ end of the 3′ terminus comprises a PS modification, optionally wherein nucleotide 4 from the 3′ end of the 3′ terminus comprises a 2′-OMe modification.

318. The gRNA of any one claims 161-317, wherein the gRNA is an sgRNA that comprises a 3′ end modification.

319. The gRNA of any one of claims 161-318, which is an sgRNA comprising a 3′ end modification, wherein the 3′ end modification is a protective 3′ end modification.

320. The gRNA of any one claims 161-319, wherein the gRNA is an sgRNA that comprises a 3′ tail.

321. The gRNA of claim 320, wherein the 3′ tail comprises 1, 2, 3, 4, 5, 6, 7, 8, 9, or 10 nucleotides.

322. The gRNA of claim 320, wherein the 3′ tail comprises about 1-2, 1-3, 1-4, 1-5, 1-7, 1-10, at least 1-5, at least 1-3, at least 1-4, at least 1-5, at least 1-5, at least 1-7, or at least 1-10 nucleotides.

323. The gRNA of any one of claims 161-322, which is an sgRNA comprising a modification in the hairpin region.

324. The gRNA of any one of claims 161-323, which is an sgRNA comprising a 3′ end modification, and a modification in the hairpin region.

325. The gRNA of claim 323 or 324, wherein the modification in the hairpin region comprises a modified nucleotide selected from 2′-O-methyl (2′-O-Me) modified nucleotide, a 2′-fluoro (2′-F) modified nucleotide, or combinations thereof.

326. The gRNA of any one of claims 323-325, wherein the modification in the hairpin region comprises or further comprises a 2′-O-methyl (2′-O-Me) modified nucleotide.

327. The gRNA of any one of claims 323-326, wherein the modification in the hairpin region comprises or further comprises a 2′-fluoro (2′-F) modified nucleotide.

328. The gRNA of any one of claims 161-327, comprising 3′ and/or 5′ protective end modification(s).

329. The gRNA of claim 328, wherein the 3′ and/or 5′ end modification comprises a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.

330. The gRNA of claim 328 or 329, wherein the 3′ and/or 5′ end modification comprises or further comprises a 2′-O-methyl (2′-O-Me) modified nucleotide.

331. The gRNA of claim 328 or 329, wherein the 3′ and/or 5′ end modification comprises or further comprises a 2′-fluoro (2′-F) modified nucleotide.

332. The gRNA of claim 328 or 329, wherein the 3′ and/or 5′ end modification comprises or further comprises a phosphorothioate (PS) linkage between nucleotides.

333. The gRNA of claim 328 or 329, wherein the 3′ and/or 5′ end modification comprises or further comprises an inverted abasic modified nucleotide.

334. The gRNA of any one any one of claims 161-333, wherein the gRNA is an sgRNA and if the sgRNA comprises a 3′ end modification, the 3′ end modification comprises any one or more of the following:
i. a modification of any one or more of the last 7, 6, 5, 4, 3, 2, or 1 nucleotides;
ii. one modified nucleotide;
iii. two modified nucleotides;
iv. three modified nucleotides;
v. four modified nucleotides;
vi. five modified nucleotides;
vii. six modified nucleotides; and
viii. seven modified nucleotides.

335. The gRNA of claim 334, wherein the 3′ end modification comprises a modification of between 1 and 7, between 1 and 5, between 1 and 4, or between 2 and 4 nucleotides.

336. The gRNA of any one of claims 161-335, wherein the gRNA is an sgRNA comprising a 3′ end modification and the 3′ end modification comprises one or more of the following:
i. a phosphorothioate (PS) linkage between nucleotides;
ii. a 2′-O-Me modified nucleotide;
iii. a 2′-O-moe modified nucleotide;
iv. a 2′-F modified nucleotide;
v. an inverted abasic modified nucleotide
vi. ENA, UNA, and/or DNA; and
vii. or a combination thereof.

337. The gRNA of any one of claims 161-336, wherein the gRNA is an sgRNA comprising a 3′ tail, and the 3′ tail comprises any one or more of:
i. a phosphorothioate (PS) linkage between nucleotides;
ii. a 2′-O-Me modified nucleotide;
iii. a 2′-O-moe modified nucleotide;
iv. a 2′-F modified nucleotide;
v. an inverted abasic modified nucleotide
vi. ENA, UNA, and/or DNA; and
vii. or a combination thereof.

338. The gRNA of claim 336, wherein the 3′ end modification comprises:
i. 1, 2, 3, 4, 5, 6, or 7 PS linkages between nucleotides;
ii. about 1-3, 1-5, 1-6, or 1-7 PS linkages between nucleotides; or
iii. PS linkages between each nucleotide.

339. The gRNA of any one of claims claim 326-328, wherein the 3′ end modification further comprises at least one 2′-O-Me, 2′-O-moe, inverted abasic, or 2′-F modified nucleotide.

340. The gRNA of any one of claims 326-329, wherein the 3′ end modification comprises at least one PS linkage, and wherein:
i. there is one PS linkage, and the linkage is between the last and second to last nucleotide;
ii. there are two PS linkages between the last three nucleotides;
iii. there are PS linkages between any one or more of the last four nucleotides;
iv. there are PS linkages between any one or more of the last five nucleotides; or
v. there are PS linkages between any one or more of the last 2, 3, 4, 5, 6, 7, 8, 9, or 10 nucleotides.

341. The gRNA of any one of claims 336-340, wherein the 3′ end modification comprises:
i. a modification of one or more of the last 1-7 nucleotides, wherein the modification is a PS linkage, inverted abasic nucleotide, 2′-O-Me, 2′-O-moe, 2′-F, or combinations thereof;
ii. a modification to the last nucleotide with 2′-O-Me, 2′-O-moe, 2′-F, or combinations thereof, and an optional one or two PS linkages to the next nucleotide and/or the first nucleotide of the 3′ tail;
iii. a modification to the last and/or second to last nucleotide with 2′-O-Me, 2′-O-moe, 2′-F, or combinations thereof, and optionally one or more PS linkages;
iv. a modification to the last, second to last, and/or third to last nucleotides with 2′-O-Me, 2′-O-moe, 2′-F, or combinations thereof, and optionally one or more PS linkages;
v. a modification to the last, second to last, third to last, and/or fourth to last nucleotides with 2′-O-Me, 2′-O-moe, 2′-F, or combinations thereof, and optionally one or more PS linkages; or
vi. a modification to the last, second to last, third to last, fourth to last, and/or fifth to last nucleotides with 2′-O-Me, 2′-O-moe, 2′-F, or combinations thereof, and optionally one or more PS linkages.

342. The gRNA of any one of claims 161-341, wherein the gRNA is an sgRNA comprising a 3′ tail, wherein the 3′ tail comprises a modification of any one or more of the nucleotides present in the 3′ tail.

343. The gRNA of claim 342, wherein the 3′ tail is fully modified.

344. The gRNA of claim 342, wherein the 3′ tail comprises 1, 2, 3, 4, 5, 6, 7, 8, 9, 10, 1-2, 1-3, 1-4, 1-5, 1-6, 1-7, 1-8, 1-9, or 1-10 nucleotides, optionally where any one or more of these nucleotides are modified.

345. The gRNA of any one of claims 336-344, wherein the 3′ end modification comprises any one or more of the following:
i. the 3′ end modification as shown in any one of SEQ ID Nos: 401-532;
ii. (i) a 2′O-Me modified nucleotide at the last nucleotide of the conserved region of an sgRNA or short-sgRNA, (ii) three consecutive 2′O-moe modified nucleotides immediately 5′ to the 2′O-Me modified nucleotide, and (iii) three consecutive PS linkages between the last three nucleotides;
iii. (i) five consecutive 2′O-Me modified nucleotides from the 3′ terminus, and (ii) three PS linkages between the last three nucleotides;
iv. an inverted abasic modified nucleotide at the last nucleotide of the conserved region of an sgRNA or short-sgRNA;
v. (i) an inverted abasic modified nucleotide at the last nucleotide of the conserved region of an sgRNA or short-sgRNA, and (ii) three consecutive 2′O-Me modified nucleotides at the last three nucleotides of the conserved region of an sgRNA or short-sgRNA;
vi. (i) 15 consecutive 2′O-Me modified nucleotides from the 3′ terminus, (ii) five consecutive 2′-F modified nucleotides immediately 5′ to the 2′O-Me modified nucleotides, and (iii) three PS linkages between the last three nucleotides;
vii. (i) alternating 2′O-Me modified nucleotides and 2′-F modified nucleotides at the last 20 nucleotides of the conserved region of an sgRNA or short-sgRNA, and (ii) three PS linkages between the last three nucleotides;
viii. (i) two or three consecutive 2′O-Me modified nucleotides, and (ii) three PS linkages between the last three nucleotides;
ix. one PS linkage between the last and next to last nucleotides; and
x. 15 or 20 consecutive 2′O-Me modified nucleotides, and (ii) three PS linkages between the last three nucleotides.

346. The gRNA of any one of claims 161-345, comprising a 5′ end modification comprising any one or more of the following:
i. a modification of any one or more of nucleotides 1-7 of the guide region;
ii. one modified nucleotide;
iii. two modified nucleotides;
iv. three modified nucleotides;
v. four modified nucleotides;
vi. five modified nucleotides;
vii. six modified nucleotides; and
viii. seven modified nucleotides.

347. The gRNA of any one of claims 161-346, comprising a 5′ end modification, wherein the 5′ end modification is a protective 5′ end modification.

348. The gRNA of any one of claims 161-347, comprising a 5′ end modification, wherein the 5′ end modification comprises a modification of between 1 and 7, between 1 and 5, between 1 and 4, between 1 and 3, or between 1 and 2 nucleotides.

349. The gRNA of any one of claims 161-348, comprising a 5′ end modification wherein the 5′ end modification comprises any one or more of the following:
i. modifications of 1, 2, 3, 4, 5, 6, or 7 of the first 7 nucleotides;
ii. modifications of about 1-3, 1-4, 1-5, 1-6, or 1-7 of the first 7 nucleotides; and
iii. modifications at the first, second, third, fourth, fifth, sixth, and/or seventh nucleotide at the 5′ end, optionally wherein the modifications are consecutive.

350. The gRNA of any one of claims 161-349, comprising a 5′ end modification, wherein the 5′ end modification comprises one or more of:
i. a phosphorothioate (PS) linkage between nucleotides;
ii. a 2′-O-Me modified nucleotide;
iii. a 2′-O-moe modified nucleotide;
iv. a 2′-F modified nucleotide;
v. an inverted abasic modified nucleotide
vi. ENA, UNA, and/or DNA; and
vii. combinations thereof.

351. The gRNA any one of claims 161-350, comprising a 5′ end modification wherein the 5′ end modification comprises:
i. 1, 2, 3, 4, 5, 6, and/or 7 PS linkages between nucleotides; or
ii. about 1-2, 1-3, 1-4, 1-5, 1-6, or 1-7 PS linkages between nucleotides.

352. The gRNA of any one of claims 161-351, wherein the sgRNA comprises a 5′ end modification and the 5′ end modification comprises at least one 2′-O-Me, 2′-O-moe, inverted abasic, 2′-H, inosine, or 2′-F modified nucleotide.

353. The gRNA of claim 352, wherein the 5′ end modification comprises at least one PS linkage, and wherein:
i. there is one PS linkage, and the linkage is at nucleotide 1 of the guide region;
ii. there are two PS linkages, and the linkages are at nucleotides 1 and 2 of the guide region;
iii. there are PS linkages at any one or more of nucleotides 1, 2, and 3 of the guide region;
iv. there are PS linkages at any one or more of nucleotides 1, 2, 3, and 4 of the guide region;
v. there are PS linkages at any one or more of nucleotides 1, 2, 3, 4, and 5 of the guide region;
vi. there are PS linkages at any one or more of nucleotides 1, 2, 3, 4, 5, and 6 of the guide region; or
vii. there are PS linkages at any one or more of nucleotides 1, 2, 3, 4, 5, 6, and 7 of the guide region.

354. The gRNA of any one of claims 352-353, wherein the 5′ end modification comprises:
i. a modification of one or more of nucleotides 1-7 of the variable region, wherein the modification is a PS linkage, inverted abasic nucleotide, 2′-O-Me, 2′-O-moe, 2′-F, 2′-H, inosine, and/or combinations thereof;
ii. a modification to the first nucleotide of the guide region with 2′-O-Me, 2′-O-moe, 2′-F, 2′-H, inosine, or combinations thereof, and an optional PS linkage to the next nucleotide;
iii. a modification to the first and/or second nucleotide of the variable region with 2′-O-Me, 2′-O-moe, 2′-F, 2′-H, inosine, or combinations thereof, and optionally one or more PS linkages;
iv. a modification to the first, second, and/or third nucleotides of the variable region with 2′-O-Me, 2′-O-moe, 2′-F, 2′-H, inosine, or combinations thereof, and optionally one or more PS linkages;
v. a modification to the first, second, third, and/or fourth nucleotides of the variable region with 2′-O-Me, 2′-O-moe, 2′-F, 2′-H, inosine, or combinations thereof, and optionally one or more PS linkages; or
vi. a modification to the first, second, third, fourth, and/or fifth nucleotides of the variable region with 2′-O-Me, 2′-O-moe, 2′-F, 2′-H, inosine, or combinations thereof, and optionally one or more PS linkages.

355. The gRNA of any one of claims 161-354, comprising a 5′ end modification, wherein the 5′ end modification comprises any one or more of the following:
i. a 5′ end modification as shown in any one of SEQ ID Nos: 1-54, 401-532, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, 3388-3430, or 3549-3552;
ii. 2′-OMe modified nucleotides at nucleotides 1, 2, and 3 of the guide region;
iii. 2′-OMe modified nucleotides at nucleotides 1, 2, and 3 of the guide region and PS linkages between nucleotides 1 and 2, 2 and 3, and 3 and 4 of the guide region;
iv. 2′-OMe modified nucleotides at nucleotides 1, 2, 3, 4, and 5 of the guide region;
v. 2′-OMe modified nucleotides at nucleotides 1, 2, 3, 4, and 5 of the guide region and PS linkages between nucleotides 1 and 2, 2 and 3, 3 and 4, 4 and 5, and 5 and 6 of the guide region;
vi. 2′O-moe modified nucleotides at nucleotides 1, 2, and 3 of the guide region;
vii. 2′O-moe modified nucleotides at nucleotides 1, 2, and 3 of the guide region and PS linkages between nucleotides 1 and 2, 2 and 3, and 3 and 4 of the guide region;
viii. an inverted abasic modified nucleotide at nucleotide 1 of the guide region;
ix. an inverted abasic modified nucleotide at nucleotide 1 of the guide region and 2′-OMe modified nucleotides at nucleotides 1, 2, and 3 of the guide region; and
x. an inverted abasic modified nucleotide at nucleotide 1 of the guide region, 2′-OMe modified nucleotides at nucleotides 1, 2, and 3 of the guide region, and PS linkages between nucleotides 1 and 2, 2 and 3, 3 and 4, 4 and 5, and 5 and 6 of the variable region.

356. The gRNA of any one of claims 161-355, wherein the gRNA is an sgRNA and the upper stem region comprises at least one modification.

357. The gRNA of claim 346, wherein the upper stem modification comprises any one or more of the following:
i. a modification to any one or more of US1-US12 in the upper stem region;
ii. a modification of at least 1, 2, 3, 4, 5, 6, 7, 8, 9, 10, 11, or all 12 nucleotides in the upper stem region; and
iii. a modification of about 1-2, 1-3, 1-4, 1-5, 1-6, 1-7, 1-8, 1-9, 1-10, or 1-12 nucleotides in the upper stem region.

358. The gRNA of any one of claims 356-357, wherein the upper stem modification comprises one or more of:
i. a 2′-O-Me modified nucleotide;
ii. a 2′-H modified nucleotide;
iii. a 2′-F modified nucleotide; and
iv. combinations thereof.

359. The gRNA of any one of claims 161-358, wherein the gRNA is an sgRNA comprising one or more modifications in the hairpin 1 region.

360. The gRNA of claim 359 wherein the sgRNA comprises a modification at H1-1.

361. The gRNA of any one of claims 161-360, wherein the gRNA is an sgRNA comprising one or more modifications in the hairpin 2 region.

362. The gRNA of claim 361, wherein the sgRNA comprises a modification at H2-1.

363. The gRNA of any one of claims 161-362, wherein the gRNA is an sgRNA comprising comprises modifications at H1-1 to H1-12.

364. The gRNA of any one of claims 161-363, wherein the gRNA is an sgRNA comprising comprises modifications at H2-1 to H2-15.

365. The gRNA of any one of claims 161-364, wherein the gRNA is an sgRNA comprising one or more modifications in each of the upper stem region, the hairpin 1 region, and the hairpin 2 region.

366. The gRNA of any one of claims 161-365, wherein the gRNA is an sgRNA comprising a modified nucleotide between hairpin 1 and hairpin 2 regions.

367. The gRNA of any one of claims 161-366, which is an sgRNA further comprising a lower stem region comprising a modification.

368. The gRNA of any one of claims 161-367, further comprising a 3′ end modification.

369. The gRNA of claim 368, wherein at least two of the last four nucleotides at the 3′ end of the 3′ terminus are modified.

370. The gRNA of claim 369, wherein at least two of the last four nucleotides at the 3′ end of the 3′ terminus are modified with 2′-O-Me, 2′-F, or 2′-O-moe.

371. The gRNA of any one of claims 368-370, further comprising phosphorothioate (PS) bonds between one or more of the last four nucleotides at the 3′ end of the 3′ terminus.

372. The gRNA of any one of claims 161-371, which is an sgRNA further comprising a bulge region comprising a modification.

373. The gRNA of any one of claims 161-372, which is an sgRNA further comprising a nexus region comprising a modification.

374. An sgRNA comprising any of SEQ ID Nos: 401-535, 601, 607-732, 801, 807-932, 1001, or 1007-1132, including the modifications of Table 1.

375. An sgRNA comprising nucleic acids having at least 99, 98, 97, 96, 95, 94, 93, 92, 91, 90, 85, 80, 75, or 70% identity to the nucleic acids of any one of SEQ ID Nos: 401-535, 601, 607-732, 801, 807-932, 1001, or 1007-1132, wherein the modification at each nucleotide of the sgRNA that corresponds to a nucleotide of the reference sequence identifier in Table 1, is identical to or equivalent to the modification shown in the reference sequence identifier in Table 1.

376. The gRNA of any one of claims 161-375, wherein the modification reduces gRNA degradation without significantly altering the ability of the guide to cleave a target nucleic acid.

377. The gRNA of any one of claims 161-376, comprising a YA modification wherein the modification comprises 2′-fluoro, 2′-H, 2′-O-Me, ENA, UNA, or PS.

378. The gRNA of any one of claims 161-377, comprising a YA modification wherein the modification alters the structure of the dinucleotide motif to reduce RNA endonuclease activity.

379. The gRNA of any one of claims 161-378, comprising a YA modification wherein the modification interferes with recognition or cleavage of a YA site by an RNase and/or stabilizes an RNA structure.

380. The gRNA of any one of claims 161-379, comprising a YA modification wherein the modification comprises one or more of:
i. a ribose modification selected from 2′-O-alkyl, 2′-F, 2′-moe, 2′-F arabinose, and 2′-H (deoxyribose);
ii. a bicyclic ribose analog, such as LNA, BNA, and ENA;
iii. an unlocked nucleic acid modification;
iv. a base modification, such as inosine, pseudouridine, and 5′-methylcytosine; and
v. an internucleoside linkage modification such as phosphorothioate.

381. The gRNA of any one of the preceding claims, wherein the gRNA comprises a guide region that comprises a modification at nucleotide 5, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.

382. The gRNA of any one of the preceding claims, wherein the gRNA comprises a guide region that comprises a modification at nucleotide 12, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.

383. The gRNA of any one of the preceding claims, wherein the gRNA comprises a guide region that comprises a 2′-OMe modification at nucleotide 5 and/or nucleotide 12, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.

384. The gRNA of any one of the preceding claims, wherein the gRNA comprises a guide region that comprises a 2′-F modification at nucleotide 5 and/or nucleotide 12, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.

385. The gRNA of any one of the preceding claims, wherein the gRNA comprises a guide region that comprises a 2′-H modification at nucleotide 5 and/or nucleotide 12, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.

386. The gRNA of any one of the preceding claims, wherein the gRNA comprises a guide region that comprises a phosphorothioate modification at nucleotide 5 and/or nucleotide 12, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.

387. The gRNA of any one of the preceding claims, wherein the gRNA comprises a guide region that comprises modifications at:
i. nucleotides 8-10;
ii. nucleotides 8 and 9;
iii. nucleotides 8 and 10; or
iv. nucleotides 9 and 10,
optionally wherein the guide region comprises 2′-OMe modifications at nucleotides 1-4, phosphorothioate modifications at nucleotides 1-3 and 6-7, and/or 2′-F modifications at nucleotides 11, 13, 14, 17, and 18.

388. The gRNA of any one of the preceding claims, wherein the gRNA comprises a guide region that comprises 2′-F modifications at:
i. nucleotides 8-10;
ii. nucleotides 8 and 9;
iii. nucleotides 8 and 10;
iv. nucleotides 9 and 10; or
v. nucleotide 8;
optionally wherein the guide region comprises 2′-OMe modifications at nucleotides 1-4, phosphorothioate modifications at nucleotides 1-3 and 6-7, and/or 2′-F modifications at nucleotides 11, 13, 14, 17, and 18.

389. The gRNA of any one of the preceding claims, wherein the gRNA comprises a guide region that comprises 2′-F modifications at:
i. nucleotides 8-10;
ii. nucleotides 8 and 9;
iii. nucleotides 8 and 10;
iv. nucleotides 9 and 10; or
v. nucleotide 8;
wherein nucleotides 8-10 do not comprise phosphorothioate modifications, and optionally wherein the guide region comprises 2′-OMe modifications at nucleotides 1-4, phosphorothioate modifications at nucleotides 1-3 and 6-7, and/or 2′-F modifications at nucleotides 11, 13, 14, 17, and 18.

390. The gRNA of any one of the preceding claims, wherein the gRNA comprises a guide region that comprises 2′-F modifications at nucleotides 8-10 and:
i. phosphorothioate modifications at 1, 2, or 3 of nucleotides 8-10;
ii. a phosphorothioate modification at nucleotide 8;
iii. a phosphorothioate modification at nucleotide 9;
iv. a phosphorothioate modification at nucleotide 10;
v. a phosphorothioate modification at nucleotides 8 and 9;
vi. a phosphorothioate modification at nucleotides 8 and 10;
vii. a phosphorothioate modification at nucleotides 9 and 10; or
viii. a phosphorothioate modification at nucleotides 8-10
optionally wherein the guide region comprises 2′-OMe modifications at nucleotides 1-4, phosphorothioate modifications at nucleotides 1-3 and 6-7, and/or 2′-F modifications at nucleotides 11, 13, 14, 17, and 18.

391. The gRNA of any one of the preceding claims, wherein the gRNA comprises a guide region that comprises:
i. a 2′-F or phosphorothioate modification at nucleotides 5 and 6;
ii. a 2′-F modification at nucleotides 5 and 6;
iii. a phosphorothioate modification at nucleotides 5 and 6;
iv. a 2′-F modification at nucleotide 5 and a phosphorothioate modification at nucleotide 6; or
v. a 2′-F modification at nucleotide 6 and a phosphorothioate modification at nucleotide 5;
optionally wherein the guide region comprises 2′-OMe modifications at nucleotides 1-4, phosphorothioate modifications at nucleotides 1-3 and 7-10, and/or 2′-F modifications at nucleotides 8-11, 13, 14, 17, and 18.

392. The gRNA of any one of the preceding claims, wherein the gRNA comprises a guide region that comprises 2′-F modifications at at least 1, 2, 3, 4, 5, or 6 of nucleotides 6-11, optionally wherein the guide region comprises 2′-OMe modifications at nucleotides 1-4, phosphorothioate modifications at nucleotides 1-3, and/or 2′-F modifications at nucleotides 13, 14, 17, and 18.

393. The gRNA of any one of the preceding claims, wherein the gRNA comprises a guide region that comprises 2′-F modifications at at least 1, 2, 3, 4, 5, 6, 7, 8, 9, or 10 of nucleotides 1-4 and 6-11, optionally wherein the guide region comprises phosphorothioate modifications at nucleotides 1-3 and/or 2′-F modifications at nucleotides 13, 14, 17, and 18.

394. The gRNA of any one of the preceding claims, wherein the gRNA comprises a guide region that comprises 2′-F modifications at nucleotides 6-11, optionally wherein the guide region comprises 2′-OMe modifications at nucleotides 1-4, phosphorothioate modifications at nucleotides 1-3, and/or 2′-F modifications at nucleotides 13, 14, 17, and 18.

395. The gRNA of any one of the preceding claims, wherein the gRNA comprises a guide region that comprises 2′-F modifications at nucleotides 1-4, optionally wherein the guide region comprises phosphorothioate modifications at nucleotides 1-3 and 6-10, and/or 2′-F modifications at nucleotides 6-11, 13, 14, 17, and 18.

396. The gRNA of any one of the preceding claims, wherein the gRNA comprises a guide region that comprises a 2′-F modification at nucleotide 9 and not a phosphorothioate modification at nucleotide 9, optionally wherein the guide region comprises 2′-OMe modifications at nucleotides 1-4, phosphorothioate modifications at nucleotides 1-3 and 6-8 and 10, and/or 2′-F modifications at nucleotides 8, 10, 11, 13, 14, 17, and 18.

397. The gRNA of any one of the preceding claims, wherein the gRNA comprises a guide region that does not comprise 2′-F modifications at at least 1, 2, 3, 4, 5, 6, 7, or 8 of nucleotides 8-11, 13, 14, 17, and 18, optionally wherein the guide region comprises 2′-OMe modifications at nucleotides 1-4 and/or phosphorothioate modifications at nucleotides 1-3 and 6-10.

398. The gRNA of any one of the preceding claims, wherein the gRNA comprises a guide region that does not comprise 2′-F modifications at nucleotides 8-11, 13, 14, 17, and 18, optionally wherein the guide region comprises 2′-OMe modifications at nucleotides 1-4 and/or phosphorothioate modifications at nucleotides 1-3 and 6-10.

399. The gRNA of any one of the preceding claims, wherein the gRNA comprises a guide region that comprises 2′-OMe modifications at at least 1, 2, 3, or 4 of nucleotides 9, 11, 13, and 14, optionally wherein the guide region comprises 2′-OMe modifications at nucleotides 1-4 and/or phosphorothioate modifications at nucleotides 1-3 and 6-10.

400. The gRNA of any one of the preceding claims, wherein the gRNA comprises a guide region that comprises 2′-OMe modifications at nucleotides 9, 11, 13, and 14, optionally wherein the guide region comprises 2′-OMe modifications at nucleotides 1-4 and/or phosphorothioate modifications at nucleotides 1-3 and 6-10.

401. The gRNA of any one of the preceding claims, wherein the gRNA comprises a guide region that comprises phosphorothioate modifications at one or both of nucleotides 8 and 10, optionally wherein the guide region comprises 2′-OMe modifications at nucleotides 1-4, phosphorothioate modifications at nucleotides 1-3 and 6-7, and/or 2′-F modifications at nucleotides 8-11, 13, 14, 17, and 18.

402. The gRNA of any one of the preceding claims, wherein the gRNA comprises a guide region that comprises modifications at at least 1, 2, 3, 4, 5, 6, 7, 8, 9, 10, 11, 12, 13, or all of the following nucleotides: 1, 2, 3, 4, 6, 7, 8, 9, 10, 11, 13, 14, 17, and 18, optionally wherein the modifications are 2′-OMe, 2′-fluoro, or phosphorothioate modifications.

403. The gRNA of any one of the the preceding claims, wherein the gRNA comprises a guide region that comprises modifications at nucleotides 1, 2, 3, 4, 6, 7, 8, 9, 10, 11, 13, 14, 17, and 18, optionally wherein the modifications are 2′-OMe, 2′-fluoro, or phosphorothioate modifications.

404. The gRNA of any one of the preceding claims, wherein 2′-OMe modifications are not present in the guide region at nucleotides 6-11 and 13-end.

405. The gRNA of any one of the preceding claims, wherein 2′-fluoro modifications are not present in the guide region at nucleotides 1-7, 15, 16, and 19-end.

406. The gRNA of any one of the preceding claims, wherein phosphorothioate modifications are not present in the guide region at nucleotides 4, 5, 11-14, 17, and 18.

407. The gRNA of any one of the preceding claims, wherein the guide region comprises an unmodified nucleotide 20.

408. The gRNA of any one of the preceding claims, wherein the guide region consists of 20 nucleotides.

409. The gRNA of any one of the preceding claims, wherein the guide region comprises a YA site at nucleotides 5-6 and a modification at nucleotide 5.

410. The gRNA of any one of the preceding claims, wherein the guide region comprises a YA site at nucleotides 12-13 and a modification at nucleotide 12.

411. The gRNA of any one of the preceding claims, wherein the guide region comprises a YA site at nucleotides 15-16 and a modification at nucleotide 15.

412. The gRNA of any one of the preceding claims, wherein the guide region comprises a YA site at nucleotides 16-17 and a modification at nucleotide 16.

413. The gRNA of any one of the preceding claims, wherein the guide region comprises a YA site at nucleotides 19-20 and a modification at nucleotide 19.

414. The gRNA of any one of the preceding claims, wherein the guide region does not comprise a YA site at nucleotides 5-6 and nucleotide 5 is unmodified.

415. The gRNA of any one of the preceding claims, wherein the guide region does not comprise a YA site at nucleotides 12-13 and nucleotide 12 is unmodified.

416. The gRNA of any one of the preceding claims, wherein the guide region does not comprise a YA site at nucleotides 15-16 and nucleotide 15 is unmodified.

417. The gRNA of any one of the preceding claims, wherein the guide region does not comprise a YA site at nucleotides 16-17 and nucleotide 16 is unmodified.

418. The gRNA of any one of the preceding claims, wherein the guide region does not comprise a YA site at nucleotides 19-20 and nucleotide 19 is unmodified.

419. The gRNA of any one of the preceding claims, wherein the gRNA comprises a guide region that comprises at least 1, 2, 3, 4, 5, 6, 7, 8, 9, 10, 11, 12, 13, or all of the following:
i. 2′-OMe and phosphorothioate modifications at nucleotide 1;
ii. 2′-OMe and phosphorothioate modifications at nucleotide 2;
iii. 2′-OMe and phosphorothioate modifications at nucleotide 3;
iv. a 2′-OMe modification at nucleotide 4;
v. a phosphorothioate modification at nucleotide 6;
vi. a phosphorothioate modification at nucleotide 7;
vii. 2′-fluoro and phosphorothioate modifications at nucleotide 8;
viii. 2′-fluoro and phosphorothioate modifications at nucleotide 9;
ix. 2′-fluoro and phosphorothioate modifications at nucleotide 10;
x. a 2′-fluoro modification at nucleotide 11;
xi. a 2′-fluoro modifications at nucleotide 13;
xii. a 2′-fluoro modifications at nucleotide 14;
xiii. a 2′-fluoro modifications at nucleotide 17; and
xiv. a 2′-fluoro modifications at nucleotide 18.

420. The gRNA of any one of the preceding claims, wherein the guide region comprises each of the modifications set forth in the preceding claim.

421. The gRNA of any one of the preceding claims, wherein the guide region comprises at least 1, 2, 3, or 4 of the following:
i. a 2′-OMe modification at nucleotide 5 if nucleotides 5 and 6 form a YA site;
ii. a 2′-OMe modification at nucleotide 12 if nucleotides 12 and 13 form a YA site;
iii. a phosphorothioate or 2′-H modification at nucleotide 15 if nucleotides 15 and 16 form a YA site;
iv. a phosphorothioate modification at nucleotide 16 if nucleotides 16 and 17 form a YA site; and
v. a phosphorothioate or 2′-fluoro modification at nucleotide 19 if nucleotides 19 and 20 form a YA site.

422. The gRNA of any one of the preceding claims, wherein the guide region comprises a YA site at nucleotides 5-6 and a a 2′-OMe modification at nucleotide 5.

423. The gRNA of any one of the preceding claims, wherein the guide region comprises a YA site at nucleotides 12-13 and a 2′-OMe modification at nucleotide 12.

424. The gRNA of any one of the preceding claims, wherein the guide region comprises a YA site at nucleotides 15-16 and a phosphorothioate modification at nucleotide 15.

425. The gRNA of any one of the preceding claims, wherein the guide region comprises a YA site at nucleotides 16-17 and a phosphorothioate modification at nucleotide 16.

426. The gRNA of any one of the preceding claims, wherein the guide region comprises a YA site at nucleotides 19-20 and a phosphorothioate modification at nucleotide 19.

427. The gRNA of any one of the preceding claims, wherein the guide region comprises a 2′-fluoro modification at nucleotide 19.

428. The gRNA of any one of the preceding claims, wherein the guide region comprises an unmodified nucleotide 15 or only a phosphorothioate modification at nucleotide 15.

429. The gRNA of any one of the preceding claims, wherein the guide region comprises an unmodified nucleotide 16 or only a phosphorothioate modification at nucleotide 16.

430. The gRNA of any one of the preceding claims, wherein the gRNA is an sgRNA comprising a conserved portion of an sgRNA comprising a hairpin region, wherein the hairpin region lacks at least 5-10 nucleotides.

431. The gRNA of claim 430, wherein the at least 5-10 lacking nucleotides are consecutive.

432. The gRNA of claim 430 or 431, wherein the at least 5-10 lacking nucleotides:
i. are within hairpin 1;
ii. are within hairpin 1 and the “N” between hairpin 1 and hairpin 2;
iii. are within hairpin 1 and the two nucleotides immediately 3′ of hairpin 1;
iv. include at least a portion of hairpin 1;
v. are within hairpin 2;
vi. include at least a portion of hairpin 2;
vii. are within hairpin 1 and hairpin 2;
viii. include at least a portion of hairpin 1 and include the “N” between hairpin 1 and hairpin 2;
ix. include at least a portion of hairpin 2 and include the “N” between hairpin 1 and hairpin 2;
x. include at least a portion of hairpin 1, include the “N” between hairpin 1 and hairpin 2, and include at least a portion of hairpin 2;
xi. are within hairpin 1 or hairpin 2, optionally including the “N” between hairpin 1 and hairpin 2;
xii. are consecutive;
xiii. are consecutive and include the “N” between hairpin 1 and hairpin 2;
xiv. are consecutive and span at least a portion of hairpin 1 and a portion of hairpin 2;
xv. are consecutive and span at least a portion of hairpin 1 and the “N” between hairpin 1 and hairpin 2; or
xvi. are consecutive and span at least a portion of hairpin 1 and two nucleotides immediately 3′ of hairpin 1.

433. The gRNA of any one of claims 430-432, wherein the at least 5-10 nucleotides comprise nucleotides 54-61 of SEQ ID NO:400, nucleotides 53-60 of SEQ ID NO:400; or nucleotides 54-58 of SEQ ID NO:400, optionally wherein the sgRNA comprises modifications at least H1-1 to H1-5 and H2-1 to H2-12.

434. The gRNA of any one of claims 430-433, wherein the at least 5-10 nucleotides:
i. consist of 5-10 nucleotides;
ii. consist of 6-10 nucleotides;
iii. consist of 5 nucleotides;
iv. consist of 6 nucleotides;
v. consist of 7 nucleotides;
vi. consist of 8 nucleotides;
vii. consist of 9 nucleotides;
viii. consist of 10 nucleotides;
ix. consist of 5-10 consecutive nucleotides;
x. consist of 6-10 consecutive nucleotides;
xi. consist of 5 consecutive nucleotides;
xii. consist of 6 consecutive nucleotides;
xiii. consist of 7 consecutive nucleotides;
xiv. consist of 8 consecutive nucleotides;
xv. consist of 9 consecutive nucleotides; or
xvi. consist of 10 consecutive nucleotides.

435. The gRNA of claim 434, wherein the at least 5-10 nucleotides comprise nucleotides 54-61 of SEQ ID NO:400, nucleotides 53-60 of SEQ ID NO:400; or nucleotides 54-58 of SEQ ID NO:400, optionally wherein the sgRNA comprises modifications at least H1-1 to H1-5 and H2-1 to H2-12.

436. The gRNA of any one of claims 430-435, wherein the at least 5-10 nucleotides:
i. comprise nucleotides 54-61 of SEQ ID NO:400;
ii. comprise nucleotides 53-60 of SEQ ID NO:400;
iii. comprise nucleotides 54-58 of SEQ ID NO:400.
iv. consist of nucleotides 54-61 of SEQ ID NO:400;
v. consist of nucleotides 53-60 of SEQ ID NO:400; or
vi. consist of nucleotides 54-58 of SEQ ID NO:400.

437. The gRNA of any one of the preceding claims, wherein the gRNA comprises modifications and/or unmodified nucleotides at at least 15 of nucleotides 1-20 from the 5′ end of the 5′ terminus that match the modification pattern at nucleotides 1-20 of any one of the gRNAs of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.

438. The gRNA of any one of the preceding claims, wherein the gRNA comprises modifications and/or unmodified nucleotides at at least 16 of nucleotides 1-20 from the 5′ end of the 5′ terminus that match the modification pattern at nucleotides 1-20 of any one of the gRNAs of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.

439. The gRNA of any one of the preceding claims, wherein the gRNA comprises modifications and/or unmodified nucleotides at at least 17 of nucleotides 1-20 from the 5′ end of the 5′ terminus that match the modification pattern at nucleotides 1-20 of a gRNA, wherein the gRNA is any one of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.

440. The gRNA of any one of the preceding claims, wherein the gRNA comprises modifications and/or unmodified nucleotides at at least 18 of nucleotides 1-20 from the 5′ end of the 5′ terminus that match the modification pattern at nucleotides 1-20 of any one of the gRNAs of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.

441. The gRNA of any one of the preceding claims, wherein the gRNA comprises modifications and/or unmodified nucleotides at at least 19 of nucleotides 1-20 from the 5′ end of the 5′ terminus that match the modification pattern at nucleotides 1-20 of any one of the gRNAs of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.

442. The gRNA of any one of the preceding claims, wherein the gRNA comprises modifications and/or unmodified nucleotides at nucleotides 1-20 from the 5′ end of the 5′ terminus that match the modification pattern at nucleotides 1-20 of any one of the gRNAs of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.

443. The gRNA of any one of the preceding claims, wherein the gRNA comprises a modification pattern that matches at least 75% of the modification pattern of any one of the gRNAs of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.

444. The gRNA of any one of the preceding claims, wherein the gRNA comprises the modification pattern of any one of the gRNAs in Table 1, wherein the modification pattern is the same as any one of the gRNAs of SEQ ID NOs: 1-54, 201-254, 301-354, 401-535, 601, 607-732, 801, 807-932, 1001, 1007-1132, 1205-1212, 1322-1406, 1417-1501, 1511-1596, 3018-3059, 3063-3104, 3108-3149, 3153-3194, 3198-3239, 3243-3284, 3295-3341, 3343-3385, or 3388-3430.

445. The gRNA of any one of claims 437-444, further comprising a sequence having at least 75% identity to the sequence of nucleotides 21-end of the gRNA.

446. The gRNA of any one of claims 437-444, further comprising a sequence having at least 80% identity to the sequence of nucleotides 21-end of the gRNA.

447. The gRNA of any one of claims 437-444, further comprising a sequence having at least 85% identity to the sequence of nucleotides 21-end of the gRNA.

448. The gRNA of any one of claims 437-444, further comprising a sequence having at least 90% identity to the sequence of nucleotides 21-end of the gRNA.

449. The gRNA of any one of claims 437-444, further comprising a sequence having at least 95% identity to the sequence of nucleotides 21-end of the gRNA.

450. The gRNA of any one of claims 437-444, further comprising a sequence having at least 98% identity to the sequence of nucleotides 21-end of the gRNA.

451. The gRNA of any one of claims 437-444, further comprising a sequence having 100% identity to the sequence of nucleotides 21-end of the gRNA.

452. An LNP composition comprising a gRNA of any one of the preceding claims.

453. A composition comprising a gRNA of any one of claims 1-451 associated with a lipid nanoparticle (LNP).

454. A composition comprising the gRNA of any one of claims 1-451, or the composition of any one of claims 452-453, further comprising a nuclease or an mRNA which encodes the nuclease.

455. The composition of claim 454, wherein the nuclease is a Cas protein.

456. The composition of claim 455, wherein the Cas protein is a Cas9.

457. The composition of claim 456, wherein the Cas9 is an S. pyogenes Cas9 or an S. aureus Cas9.

458. The composition of any one of claims 453-457, wherein the nuclease is a nickase or a dCas.

459. The composition of any one of claims 453-458, wherein the nuclease is modified.

460. The composition of claim 459 wherein the modified nuclease comprises a nuclear localization signal (NLS).

461. The composition of any one of claims 452-460, comprising an mRNA which encodes the nuclease.

462. The composition of claim 461, wherein the mRNA comprises the sequence of any one of SEQ ID NOs: 3499-3527 or 3529-3546.

463. A pharmaceutical formulation comprising the gRNA of any one of claims 1-451 or the composition of any one of claims 452-462 and a pharmaceutically acceptable carrier.

464. A method of modifying a target DNA comprising, delivering a Cas protein or a nucleic acid encoding a Cas protein, and any one or more of the following to a cell:
i. the gRNA of any one of claims 1-451;
ii. the composition of any one of claims 452-462; and
iii. the pharmaceutical formulation of claim 463.

465. The method of claim 464, wherein the method results in an insertion or deletion in a gene.

466. The method of claim 464 or claim 465, further comprising delivering to the cell a template, wherein at least a part of the template incorporates into a target DNA at or near a double strand break site induced by the Cas protein.

467. The gRNA of any one of claims 1-451, the composition of claims 452-462, or the pharmaceutical formulation of claim 463 for use in preparing a medicament for treating a disease or disorder.

468. Use of the gRNA of any one of claims 1-451, the composition of claims 452-462, or the pharmaceutical formulation of claim 463 in the manufacture of a medicament for treating a disease or disorder.
